(19) United States
(12) Patent Application Publication
(10) Pub. No.: US 2023/0146675 A1
(43) Pub. Date: May 11, 2023

(54) PYRIMIDO[5,4-d]PYRIMIDINE DERIVATIVES AS ENT INHIBITORS FOR THE TREATMENT OF CANCERS, AND COMBINATION THEREOF WITH ADENOSINE RECEPTOR ANTAGONISTS

(71) Applicant: ITEOS BELGIUM SA, Gosselies (BE)

(72) Inventors: Michael Deligny, Rhode-Saint-Genese (BE); Stefano Crosginani, Vevey (CH); Erica Joke Katelijne Houthuys, Enghien (BE)

(21) Appl. No.: 17/895,667

(22) Filed: Aug. 25, 2022

Related U.S. Application Data
(63) Continuation of application No. PCT/EP2021/054821, filed on Feb. 26, 2021.
(60) Provisional application No. 62/981,977, filed on Feb. 26, 2020.

(30) Foreign Application Priority Data
May 7, 2020 (EP) 20173499.3

(51) Int. Cl. C07D 487/04; C07D 513/14

(52) U.S. Cl. CPC C07D 487/04; C07D 513/14

(57) ABSTRACT

The present invention relates to pyrimido[5,4-d]pyrimidine derivatives of formula I, including pharmaceutically acceptable salts and solvates thereof.

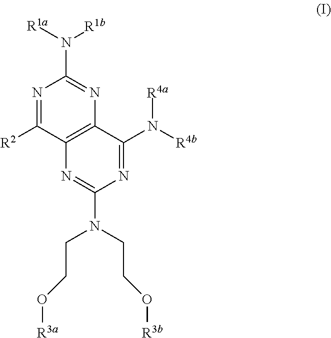

Compounds of the invention are inhibitors of ENT family transporter, especially of ENT1, and are useful as therapeutic compounds for the treatment of cancers. The invention also relates to the combined use of the pyrimido[5,4-d]pyrimidine derivatives with an adenosine receptor antagonist, for the treatment of cancers.

### FIELD OF INVENTION

The present invention relates to pyrimido[5,4-d]pyrimidine derivatives, including pharmaceutically acceptable salts and solvates thereof. Compounds of the invention are inhibitors of ENT family transporter, especially of ENT1, and are useful as therapeutic compounds, especially in the treatment of cancers. The invention also relates to the combined use of the pyrimido[5,4-d]pyrimidine derivatives of the invention with an adenosine receptor antagonist, for the treatment of cancers.

### BACKGROUND OF INVENTION

The equilibrative nucleoside transporter (ENT) family, also known as SLC29, is a group of plasmalemmal transport proteins which transport nucleoside substrates into cells. There are four known ENTs, designated ENT1, ENT2, ENT3, and ENT4.

One of the endogenous substrates for ENTs is adenosine, a potent physiological and pharmacological regulator of numerous functions. Cellular signaling by adenosine occurs through four known G-protein-coupled adenosine receptors A1, A2A, A2B, and A3. By influencing the concentration of adenosine available to these receptors, ENTs fulfil important regulatory roles in different physiological processes, such as modulation of coronary blood flow, inflammation, and neurotransmission (Griffith D A and Jarvis S M, Biochim Biophys Acta, 1996, 1286, 153-181; Shryock J C and Belardinelli L, Am J Cardiol, 1997, 79(12A), 2-10; Anderson C M et al., J Neurochem, 1999, 73, 867-873).

A variety of drugs such as dilazep, dipyridamole, and draflazine interact with ENTs and alter adenosine levels, and were developed for their cardioprotective or vasodilatory effects.

Adenosine is also a potent immunosuppressive metabolite that is often found elevated in the extracellular tumor microenvironment (TME) (Blay J et al., Cancer Res, 1997, 57, 2602-2605). Extracellular adenosine is generated mainly by the conversion of ATP by the ectonucleotidases CD39 and CD73 (Stagg J and Smyth M J, Oncogene, 2010, 2, 5346-5358). Adenosine activates four G-protein-coupled receptor subtypes (A1, A2A, A2B, and A3). In particular, activation of the A2A receptor is believed to be the main driver of innate and adaptive immune cell suppression leading to suppression of antitumor immune responses (Ohta and Sitkovsky, Nature, 2001, 414, 916-920) (Stagg and Smyth, Oncogene, 2010, 2, 5346-5358) (Antonioli L et al., Nature Reviews Cancer, 2013, 13, 842-857) (Cekic C and Linden J, Nature Reviews, Immunology, 2016, 16, 177-192) (Allard B et al., Curr Op Pharmacol, 2016, 29, 7-16) (Vijayan D et al., Nature Reviews Cancer, 2017, 17, 709-724).

The Applicant previously evidenced in PCT/EP2019/076244 that adenosine as well as ATP profoundly suppress T cell proliferation and cytokine secretion (IL-2), and strongly reduce T cell viability. Adenosine- and ATP-mediated suppression of T cell viability and proliferation were successfully restored by using ENTs inhibitors. Moreover, the use of an ENT inhibitor in combination with an adenosine receptor antagonist enabled to restore not only adenosine- and ATP-mediated suppression of T cell viability and proliferation, but also restored T cell cytokine secretion. These results showed that ENTs inhibitors either alone or in combination with an adenosine receptor antagonist may be useful for the treatment of cancers.

As mentioned above dipyridamole is known as ENT inhibitor. Dipyridamole had a promising potency in vitro, especially with regard to ENT1 (IC₅₀ equal to 542 nM in assay conditions containing 2% human serum albumin). Further to cardioprotective or vasodilatory effects, dipyridamole was also tested to potentiate the activity of antimetabolite anticancer drugs. Nevertheless, it was shown that dipyridamole has a huge binding to α1-acid glycoprotein (AGP—also referred to as AAG), an acute phase protein and important drug-binding protein (MacGregor TR, J Pharm Sci 1991). Such binding to AGP leads to a loss of potency for dipyridamole, since it is less available under free form. This was confirmed in assay conditions containing physiological concentrations of AGP: dipyridamole has an IC₅₀ equal to 542 nM in assays without AGP, which drop to an IC₅₀ equal to 2470 nM in assays containing 0.06% AGP. Moreover, AGP has been shown to be elevated up to 5-fold in the plasma during an acute phase response, the systemic answer to local inflammation (Fournier T, Biochim Biophys Acta 2000). Consequently, AGP is also increased in the plasma of cancer patients (Jackson P R, Clin Pharmacol Ther 1982; Piver M S, Gynecol Oncol 1988; Ohbatake Y, Clin Exp Med 2016). Therefore, at the approved dose, if used in cancer patients, dipyridamole is not sufficiently available in free from to be able to inhibit ENT1 for more than 90% throughout the day. Attempts were conducted to provide analogs of dipyridamole having a lower binding to AGP than dipyridamole, in order to retain a higher free fraction of the compound in plasma (Curtin et al., British Journal of Cancer, 1999, 80(11), 1738-1746). Nevertheless, compounds tested by Curtin et al. displayed a lower activity with regards to ENT1 inhibition compared to dipyridamole.

Other analogs of dipyridamole were proposed in order to improve ENT1 and ENT2 inhibition (Lin et al., J. Med. Chem., 2007, 50, 3906-3920). Nevertheless, the binding of these analogs to AGP was not studied.

Therefore, there is still a need for more potent ENTs inhibitors, and especially ENT1 inhibitors, which have a low binding to AGP—which enables to ensure a high free fraction of the compound in plasma—to be used for the treatment of cancers, either alone or in combination with an adenosine receptor antagonist.

For that purpose, the Applicant herein provides the pyrimido[5,4-d]pyrimidine derivatives of formula I detailed below.

### SUMMARY

This invention thus relates to a compound of formula I:

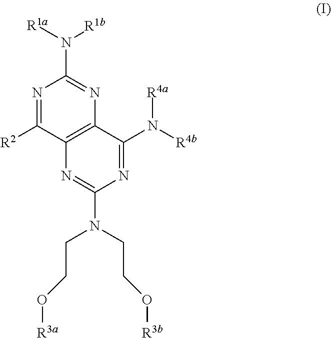

or a pharmaceutically acceptable salt or solvate thereof, wherein R^{1a}, R^{1b}, R², R^{3a}, R³, R^{4a} and R^{4b} are hereafter defined.

According to one embodiment, the compound of the invention is of formula Ia or Ia1 as defined hereafter. Preferably, the compound of the invention is selected from the compounds listed in Table 1 hereafter.

The present invention further relates the compound of formula I of the invention, for use as a medicament. Especially, it relates the compound of formula I of the invention, for use in the treatment of cancer.

The present invention also relates to a pharmaceutical composition comprising a compound according to the invention and at least one pharmaceutically acceptable excipient.

In one embodiment, the pharmaceutical composition according to the invention, further comprises an adenosine receptor antagonist. Especially, the invention provides a pharmaceutical composition comprising:
(a) an effective amount of a compound according to the invention;
(b) an effective amount of an adenosine receptor antagonist; and
(c) at least one pharmaceutically acceptable excipient.

In one embodiment, the adenosine receptor antagonist is an A2A or A2B receptor antagonist.

In one embodiment, the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide.

In another embodiment, the adenosine receptor antagonist is the adenosine receptor antagonist is a compound of Formula (II):

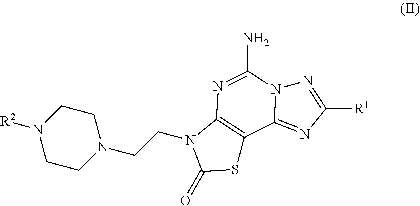

or a pharmaceutically acceptable salt or solvate thereof, wherein R¹ and R² are as defined hereafter.

The invention further relates to a combination comprising:
(a) an effective amount of a compound according to the invention; and
(b) an effective amount of an adenosine receptor antagonist.

In one embodiment, in the combination, the adenosine receptor antagonist is an A2A or A2B receptor antagonist, and is preferably selected among those listed above.

The invention further relates to a kit of parts comprising:
(a) a first part comprising an effective amount of a compound of formula I according to the invention; and
(b) a second part comprising an effective amount of an adenosine receptor antagonist.

In one embodiment, in the kit of parts, the adenosine receptor antagonist is an A2A or A2B receptor antagonist, and is preferably selected among those listed above.

The invention also relates to the combination, the pharmaceutical composition or the kit of parts according to the invention, for use in the treatment of cancer. In one embodiment, the compound according to the invention is administered prior to, concomitant with, or subsequent to the administration of the adenosine receptor antagonist.

The invention further relates to a method of inhibiting ENT1 in a patient need thereof, comprising: administering to said patient an effective amount of a compound of formula I according to the invention.

The invention also relates to method of treating cancer in a patient need thereof, comprising: administering to said patient an effective amount of a compound of formula I according to the invention.

The invention is also directed to a method of treating cancer in a patient need thereof, comprising: administering to said patient a combination of a compound of formula I according to the invention and an adenosine receptor antagonist. In one embodiment, the compound of formula I according to the invention is administered prior to, concomitant with, or subsequent to administration of the adenosine receptor antagonist. In one embodiment, the adenosine receptor antagonist is an A2A or A2B receptor antagonist. In one embodiment, the adenosine receptor antagonist is selected among those listed above.

### Definitions

In the present invention, the following terms have the following meanings:

The term “aldehyde” refers to a group —CHO.

The term “alkenyl” refers to unsaturated hydrocarbyl group, which may be linear or branched, comprising one or more carbon-carbon double bonds. Suitable alkenyl groups comprise between 2 and 6 carbon atoms, preferably between 2 and 4 carbon atoms, still more preferably between 2 and 3 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and the like.

The term “alkenylcarbonyl” refers to a group —(C═O)-alkenyl wherein alkenyl is as herein defined.

The term “alkenylcarbonylamino” refers to a group —NH—(C═O)-alkenyl wherein alkenyl is as herein defined.

The term “alkoxy” refers to a group —O-alkyl wherein alkyl is as herein defined.

The term “alkyl” refers to a hydrocarbyl radical of formula CₙH₂ₙ₊₁ wherein n is a number greater than or equal to 1. Generally, alkyl groups of this invention comprise from 1 to 8 carbon atoms, more preferably, alkyl groups of this invention comprise from 1 to 6 carbon atoms. Alkyl groups may be linear or branched. Suitable alkyl groups include methyl, ethyl, propyl, butyl, pentyl, hexyl, heptyl and octyl.

The term “alkylaminoalkyl” refers to a group -alkyl-NH-alkyl wherein alkyl is as herein defined.

The term “alkylaminoalkylaminocarbonyl” refers to a group —(C═O)—NH-alkyl-NH-alkyl wherein alkyl is as herein defined.

The term “(alkylaminoalkyl)(alkyl)aminocarbonyl” refers to a group —(C═O)—NR¹R² wherein R¹ is an alkyl group and R² is a -alkyl-NH-alkyl group, wherein alkyl is as herein defined.

The term “alkylaminoalkylcarbonyl” refers to a group —(C═O)-alkyl-NH-alkyl wherein alkyl is as herein defined.

The term “alkylcarbonyl” refers to a group —(C═O)-alkyl wherein alkyl is as herein defined.

The term “alkylcarbonylamine” refers to a group —NH—(C═O)-alkyl wherein alkyl is as herein defined.

The term “alkylcarbonyloxyalkyl” refers to a group -alkyl-O—(C═O)-alkyl wherein alkyl is as herein defined.

The term “alkylheteroaryl” refers to any heteroaryl substituted by an alkyl group wherein alkyl is as herein defined.

The term “alkyloxyalkyl” refers to a group -alkyl-O-alkyl wherein alkyl is as herein defined.

The term “alkyloxyalkyloxy” refers to a group —O-alkyl-O-alkyl wherein alkyl is as herein defined.

The term “alkyloxycarbonyl” refers to a group —(C═O)—O-alkyl wherein alkyl is as herein defined.

The term “alkylsulfonyl” refers to a group —SO₂-alkyl wherein alkyl is as herein defined.

The term “alkylsulfonylaminoalkyl” refers to a group -alkyl-NH—SO₂-alkyl wherein alkyl is as herein defined.

The term “alkylsulfonealkyl” refers to a group -alkyl-SO₂-alkyl wherein alkyl is as herein defined.

The term “alkylsulfonimidoyl” refers to a group —S(═O)(═NH)-alkyl wherein alkyl is as herein defined.

The term “alkylsulfoxide” refers to a group —(S═O)-alkyl wherein alkyl is as herein defined.

The term “alkylsulfoxidealkyl” refers to a group -alkyl-SO-alkyl wherein alkyl is as herein defined.

The term “alkyne” refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkyl groups. Non-limiting examples of alkynyl groups are ethynyl, 2-propynyl, 2-butynyl, 3-butynyl, 2-pentynyl and its isomers, 2-hexynyl and its isomers and the like.

The term “alkynealkyl” refers to a group -alkyl-alkyne wherein alkyl and alkyne are as herein defined.

The term “amino” refers to a group —NH₂.

The term “aminoalkyl” refers to a group -alkyl-NH₂ wherein alkyl is as herein defined.

The term “aminoalkylaminocarbonyl” refers to a group —(C═O)—NH-alkyl-NH₂ wherein alkyl is as herein defined.

The term “aminoalkylcarbonylamino” refers to a group —NH—(C═O)-alkyl-NH₂ wherein alkyl is as herein defined.

The term “aminocarbonyl” or “aminocarboxy” refers to a group —(C═O)—NH₂.

The term “(aminocarbonylalkyl)(alkyl)amino” refers to a group —NR¹R² wherein R¹ is an alkyl group and R² is a -alkyl-(C═O)—NH₂ group, wherein alkyl is as herein defined.

The term “aminocarbonylalkylamino” refers to a group —NH-alkyl-(C═O)—NH₂ wherein alkyl is as herein defined.

The term “aminosulfonyl” refers to a group —SO₂—NH₂.

The term “aryl” refers to a polyunsaturated, aromatic hydrocarbyl group having a single ring (i.e. phenyl) or multiple aromatic rings fused together (e.g. naphtyl), typically containing 5 to 12 atoms; preferably 5 to 10; more preferably the aryl is a 5- or 6-membered aryl. Non-limiting examples of aryl comprise phenyl, naphthalenyl.

The term “arylalkyl” refers to a group -alkyl-aryl wherein alkyl and aryl are as herein defined.

The term “aryloxyalkyl” refers to a group -alkyl-O-aryl wherein alkyl and aryl are as herein defined.

The term “carbonyl” refers to a group —(C═O)—.

The term “carbonylamino” refers to a group —NH—(C═O)—.

The term “cyano” refers to a group —CN.

The term “cyano” refers to a group -alkyl-CN.═ wherein alkyl is as herein defined.

The term “cycloalkyl” refers to a cyclic alkyl group, that is to say, a monovalent, saturated, or unsaturated hydrocarbyl group having 1 or 2 cyclic structures. Cycloalkyl includes monocyclic or bicyclic hydrocarbyl groups. Cycloalkyl groups may comprise 3 or more carbon atoms in the ring and generally, according to this invention comprise from 3 to 10, more preferably from 3 to 8 carbon atoms; still more preferably more preferably the cycloalkyl is a 5- or 6-membered cycloalkyl. Examples of cycloalkyl groups include but are not limited to cyclopropyl, cyclobutyl, cyclopentyl, cyclohexyl.

The term “cycloalkyloxy” refers to a group —O-cycloalkyl wherein cycloalkyl is as herein defined.

The term “dialkylamino” refers to a group —NR¹R² wherein R¹ and R² are both independently alkyl group as herein defined.

The term “dialkylaminoalkyl” refers to a group -alkyl-NR¹R² wherein R¹ and R² are both independently alkyl group, as herein defined.

The term “dialkylaminoalkylaminocarbonyl” refers to a group —(C═O)—NH-alkyl-NR¹R² wherein R¹ and R² are both alkyl group, as herein defined.

The term “dialkylaminoalkylcarbonyl” refers to a group —(C═O)-alkyl-NR¹R² wherein R¹ and R² are both alkyl group, as herein defined.

The term “dihydroxyalkyl” refers to a group alkyl is as herein defined substituted by two hydroxyl (—OH) groups.

The term “halo” or “halogen” refers to fluoro, chloro, bromo, or iodo.

The term “haloalkyl” refers to an alkyl group in which one or more hydrogen atom is replace by a halogen atom.

The term “haloalkyloxy” refers to a group —O-haloalkyl wherein alkyl is as herein defined.

The term “heteroaryl” refers to an aryl group as herein defined wherein at least one carbon atom is replaced with a heteroatom. In other words, it refers to 5 to 12 carbon-atom aromatic single rings or ring systems containing 2 rings which are fused together, typically containing 5 to 6 atoms; in which one or more carbon atoms is replaced by oxygen, nitrogen and/or sulfur atoms where the nitrogen and sulfur heteroatoms may optionally be oxidized and the nitrogen heteroatoms may optionally be quaternized. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, pyridinyl, pyridazinyl, pyrimidinyl and pyrazinyl.

The term “heteroarylalkyl” refers to a group -alkyl-heteroaryl wherein alkyl and heteroaryl are as herein defined.

The term “heterocyclyl” or “heterocycle” refers to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Preferably the heterocyclyl is a 5- or 6-membered heterocyclyl. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. Non limiting exemplary heterocyclic groups include piperidinyl, piperazinyl, azetidinyl, azocanyl, diazepanyl, diazocanyl, morpholin-4-yl, oxazepanyl, pyrrolidinyl, thiomorpholin-4-yl, tetrahydrofuranyl, tetrahydropyranyl, aziridinyl, oxiranyl, thiiranyl, 2-imidazolinyl, pyrazolidinyl imidazolidinyl, isoxazolinyl, oxazolidinyl, isoxazolidinyl, thiazolidinyl, isothiazolidinyl, succinimidyl, 3H-indolyl, indolinyl, isoindolinyl, 2H-pyrrolyl, 1-pyrrolinyl, 2-pyrrolinyl, 3-pyrrolinyl, 4H-quinolizinyl, 2-oxopiperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, oxetanyl, thietanyl, 3-dioxolanyl, 1,4-dioxanyl, 2,5-dioximidazolidinyl, 2-oxopiperidinyl, 2-oxopyrrolodinyl, indolinyl, tetrahydrothiophenyl, tetrahydroquinolinyl, tetrahydroisoquinolinyl, 1-oxido-1-thiomorpholin-4-yl, 1-dioxido-1-thiomorpholin-4-yl, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, dihydrotriazolopyrazine, dihydroimidazopyrazine, hexahydropyrrolopyrrole, hexahydropyrrolopyrazine.

The term “heterocyclylalkyl” refers to a group -alkyl-heterocyclyl wherein alkyl and heterocyclyl are as herein defined.

The term “heterocyclylalkylaminocarbonyl” refers to a group —(C═O)—NH-alkyl-heterocyclyl, wherein alkyl and heterocyclyl are as herein defined.

The term “(heterocyclyl)(alkyl)aminoalkyl” refers to a group -alkyl-NR¹R² wherein R¹ is an alkyl group and R² is a heterocyclyl group, wherein alkyl and heterocyclyl are as herein defined.

The term “heterocyclylalkyloxyalkyl” refers to a group -alkyl-O-alkyl-heterocyclyl wherein alkyl and heterocyclyl are as herein defined.

The term “heterocyclylcarbonyl” refers to a group —(C═O)-heterocyclyl wherein heterocyclyl is as herein defined.

The term “heterocyclyloxy” to a group —O-heterocyclyl wherein heterocyclyl is as herein defined.

The term “heterocyclylsulfonyl” refers to a group —SO₂-heterocyclyl wherein heterocyclyl is as herein defined.

The term “hydroxy” or “hydroxyl” refers to a group —OH.

The term “hydroxyalkyl” refers to a group -alkyl-OH wherein alkyl is as herein defined.

The term “hydroxyalkylaminoalkyl” refers to a group -alkyl-NH-alkyl-OH wherein alkyl is as herein defined.

The term “hydroxycarbonyl” refers to a group —C(═O)—OH wherein carbonyl is as herein defined. In other words, “hydroxycarbonyl” corresponds to a carboxylic acid group.

The term “oxo” refers to a ═O substituent.

The term “sulfonylamino” refers to a group —NH—SO₂.

The term “intermediate” or “intermediate compound” refers to a compound which is produced in the course of a chemical synthesis, which is not itself the final product, but is used in further reactions which produce the final product. There may be many different intermediate compounds between the starting material and end product in the course of a complex synthesis.

The term “about”, preceding a figure encompasses plus or minus 10%, or less, of the value of said figure. It is to be understood that the value to which the term “about” refers is itself also specifically, and preferably, disclosed.

The term “administration”, or a variant thereof (e.g. “administering”), means providing the active agent or active ingredient, alone or as part of a pharmaceutically acceptable composition, to the patient in whom/which the condition, symptom, or disease is to be treated or prevented.

The term “antagonist” refers to a natural or synthetic compound which binds to the protein and blocks the biological activation of the protein, and thereby the action of the said protein. The protein may be a receptor, i.e. a protein molecule that receives chemical signals from outside a cell. Consequently, “an adenosine receptor antagonist” includes any chemical entity that, upon administration to a patient, results in inhibition or down-regulation of a biological activity associated with activation of an adenosine receptor in the patient, including any of the downstream biological effects otherwise resulting from the binding to an adenosine receptor of its natural ligand. Such adenosine receptor antagonists include any agent that can block activation of an adenosine receptor or any of the downstream biological effects of an adenosine receptor activation.

The term “inhibitor” refers to a natural or synthetic compound that has a biological effect to inhibit or significantly reduce or down-regulate the expression of a gene and/or a protein or that has a biological effect to inhibit or significantly reduce the biological activity of a protein. Consequently, an “ENT inhibitor” or «inhibitor of an ENT family transporter” refers to a compound that has a biological effect to inhibit or significantly reduce or down-regulate the biological activity of ENT family transporter.

The term “chemotherapy” refers to a type of cancer treatment that uses one or more anticancer drugs (chemotherapeutic agents) as part of a standardized chemotherapy regimen. Chemotherapy may be given with a curative intent or it may aim to prolong life or to reduce symptoms. Chemotherapeutic agents are for example selected from anticancer alkylating agents, anticancer antimetabolites, anticancer antibiotics, plant-derived anticancer agents, anticancer platinum coordination compounds and any combination thereof.

The term “hormone therapy” refers to the use of hormones in medical treatment. In one embodiment, the hormone therapy is oncologic hormone therapy.

The term “human” refers to a subject of both genders and at any stage of development (i.e. neonate, infant, juvenile, adolescent, adult).

The term “patient” refers to a mammal, more preferably a human, who/which is awaiting the receipt of, or is receiving medical care or is/will be the object of a medical procedure.

The term “immunotherapy” refers to a therapy aiming at inducing and/or enhancing an immune response towards a specific target, for example towards cancer cells.

Immunotherapy may involve the use of checkpoint inhibitors, checkpoint agonists (also called T-cell agonists), IDO inhibitors, PI3K inhibitors, adenosine receptor inhibitors, adenosine-producing enzymes inhibitors, adoptive transfer, therapeutic vaccines, and combinations thereof.

The expression “pharmaceutically acceptable” refers to the ingredients of a pharmaceutical composition are compatible with each other and not deleterious to the subject to which it is administered.

The expression “pharmaceutically acceptable carrier, diluent, excipient and/or adjuvant” refers to a substance that does not produce an adverse, allergic or other untoward reaction when administered to an animal, preferably a human. It includes any and all inactive substance such as for example solvents, cosolvents, antioxidants, surfactants, stabilizing agents, emulsifying agents, buffering agents, pH modifying agents, preserving agents (or preservating agents), antibacterial and antifungal agents, isotonifiers, granulating agents or binders, lubricants, disintegrants, glidants, diluents or fillers, adsorbents, dispersing agents, suspending agents, coating agents, bulking agents, release agents, absorption delaying agents, sweetening agents, flavoring agents and the like. For human administration, preparations should meet sterility, pyrogenicity, general safety and purity standards as required by regulatory offices, such as, e.g., FDA Office or EMA.

The terms “prevent”, “preventing” and “prevention”, as used herein, refer to a method of delaying or precluding the onset of a condition or disease and/or its attendant symptoms, barring a patient from acquiring a condition or disease, or reducing a patient's risk of acquiring a condition or disease.

The term “prodrug” as used herein means the pharmacologically acceptable derivatives of compounds of Formula (I), such as for example esters or amides, whose in vivo biotransformation product generates the biologically active drug. Prodrugs are generally characterized by increased bio-availability and are readily metabolized into biologically active compounds in vivo.

The term “radiation therapy” refers to a method of treatment of cancer employing various radiations such as X-ray, gamma-ray, neutron ray, electron beam, proton beam and radiation sources. It is used as part of cancer treatment to control or kill malignant cells. Radiation therapy may be curative in a number of types of cancer if they are localized to one area of the body. It may also be used as part of adjuvant therapy, to prevent tumor recurrence after surgery to remove a primary malignant tumor. The three main divisions of radiation therapy are: external beam radiation therapy (EBRT or XRT); brachytherapy or sealed source radiation therapy; and systemic radioisotope therapy (RIT) or unsealed source radiotherapy.

The terms “therapeutically effective amount” or “effective amount” or “therapeutically effective dose” refer to the amount or dose of active ingredient that is aimed at, without causing significant negative or adverse side effects to the subject, (1) delaying or preventing the onset of a cancer in the subject; (2) reducing the severity or incidence of a cancer; (3) slowing down or stopping the progression, aggravation, or deterioration of one or more symptoms of a cancer affecting the subject; (4) bringing about ameliorations of the symptoms of a cancer affecting the subject; or (5) curing a cancer affecting the subject. A therapeutically effective amount may be administered prior to the onset of a cancer for a prophylactic or preventive action. Alternatively, or additionally, a therapeutically effective amount may be administered after initiation of a cancer for a therapeutic action.

The terms “treating” or “treatment” refer to therapeutic treatment; wherein the object is to prevent or slow down the targeted pathologic condition or disease. A subject or mammal is successfully “treated” for a disease or affection or condition if, after receiving the treatment according to the present invention, the subject or mammal shows observable and/or measurable reduction in or absence of one or more of the following: reduction of the number of cancer cells; and/or relief to some extent, for one or more of the symptoms associated with the specific disease or condition; reduced morbidity and mortality, and improvement in quality of life issues. The above parameters for assessing successful treatment and improvement in the disease are readily measurable by routine procedures familiar to a physician.

The term “stem cell transplant” refers to a procedure in which a patient receives healthy blood-forming cells (stem cells) to replace their own that have been destroyed by disease or by the radiation or high doses of anticancer drugs that are given as part of the procedure. The healthy stem cells may come from the blood or bone marrow of the patient, from a donor, or from the umbilical cord blood of a newborn baby. A stem cell transplant may be autologous (using a patient's own stem cells that were collected and saved before treatment), allogeneic (using stem cells donated by someone who is not an identical twin), or syngeneic (using stem cells donated by an identical twin).

The term “subject” refers to a mammal, preferably a human. In one embodiment, the subject is diagnosed with a cancer. In one embodiment, the subject is a patient, preferably a human patient, who/which is awaiting the receipt of, or is receiving, medical care or was/is/will be the subject of a medical procedure or is monitored for the development or progression of a disease, such as a cancer. In one embodiment, the subject is a human patient who is treated and/or monitored for the development or progression of a cancer. In one embodiment, the subject is a male. In another embodiment, the subject is a female. In one embodiment, the subject is an adult. In another embodiment, the subject is a child.

### DETAILED DESCRIPTION

Compounds—ENT Inhibitors

The present disclosure thus provides pyrimido[5,4-d]pyrimidine derivatives, which may be useful as ENT inhibitors. In one embodiment, the present disclosure thus provides compounds of formula A:

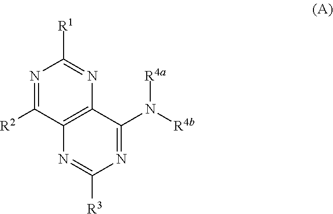

or a pharmaceutically acceptable salt or solvate thereof, wherein
R¹ is —OR^{1a} or NR^{1a}R^{1b},
   R^{1a} and R^{1b} represent each independently hydrogen, alkyl optionally substituted with one or more of hydroxy, halo, oxo, amino, —NHS(O)₂NR⁵₂, and alkylsulfonyl, arylalkyl wherein the aryl part of the arylalkyl is substituted by one or more alkyl, haloalkyl, halogen, alkoxy or —CO₂H group, heteroarylalkyl, heterocyclylalkyl wherein the heterocyclyl part of the heterocyclylalkyl is optionally substituted by one or more alkyl group, aminocarbonylalkyl, alkylcarbonylaminoalkyl, alkyloxyalkyl, alkylcarbonyloxyalkyl, or heterocyclylalkyloxyalkyl; with the condition that R^{1a} and R^{1b} are not both hydrogen;
   or R^{1a} and R^{1b} are linked together and form with the nitrogen atom to which they are attached a heterocycle selected from piperidine and piperazine, wherein the heterocycle is optionally substituted with one or more substituent selected from alkyl, alkylcarbonyl, alkyloxycarbonyl, alkylsulfonyl, halo, hydroxy, optionally substituted heteroaryl, and oxo; or the heterocycle is fused with a group selected from aryl and heteroaryl, wherein the aryl or heteroaryl group is optionally substituted with alkyl or alkoxy;
R² represents —NR^{2a}R^{2b} or —OR^{2c}; wherein
   R^{2a} and R^{2b} represent each independently hydrogen, alkyl, alkyloxyalkyl, alkylsulfonylaminoalkyl, arylalkyl wherein the aryl part of the arylalkyl is optionally substituted by one or more of alkoxy, alkylsulfonyl, aminosulfonyl, aminocarbonyl, cyano, halo, haloalkyloxy, optionally substituted heteroaryl, sulfoxide and sulfonylamine groups, aryloxyalkyl, cyanoalkyl, cycloalkyl, heteroarylalkyl wherein the heteroaryl part of the heteroarylalkyl is optionally substituted by one or more of alkyl, halo, haloalkyl and NH₂ groups, heterocyclylalkyl or hydroxyalkyl;
   or R^{2a} and R^{2b} are linked together and form with the nitrogen atom to which they are attached a heterocycle, wherein the heterocycle is optionally substituted with one or more substituent selected from alkoxy, alkyl, alkylcarbonyl, alkylcarbonylamine, alkyloxyalkyl, alkyloxyalkyloxy, alkyloxycarbonyl, alkylsulfonyl, aminocarbonyl, cycloalkyl, cyano, halo, haloalkyl, heteroaryl (optionally substituted by one or more of alkyl, cyano and NH₂ groups), hydroxy, hydroxyalkyl and oxo; or two substituents present on a same carbon atom of the heterocycle are linked together and form a spiro heterocycle; or the heterocycle is fused with a group selected from aryl and heteroaryl, wherein the aryl or heteroaryl group is optionally substituted with alkyl or alkoxy; and
   R^{2c} represents arylalkyl wherein the aryl part of the arylalkyl is optionally substituted by one or more of alkoxy, cyano and halo groups; or heteroarylalkyl wherein the heteroaryl part of the heteroarylalkyl is optionally substituted by one or more of alkyl and cyano groups;
R³ is —NR^{3A}R^{3B},
   R^{3A} and R^{3B} represent each independently alkyl optionally substituted with one or more of hydroxy, halo, oxo, amino, —NHS(O)₂NR⁵₂, and alkylsulfonyl, arylalkyl wherein the aryl part of the arylalkyl is substituted by one or more alkyl, haloalkyl, halogen, alkoxy or —CO₂H group, heteroarylalkyl, heterocyclylalkyl wherein the heterocyclyl part of the heterocyclylalkyl is optionally substituted by one or more alkyl group, aminocarbonylalkyl, alkylcarbonylaminoalkyl, alkyloxyalkyl, alkylcarbonyloxyalkyl or heterocyclylalkyloxyalkyl; with the condition that R^{3A} and R^{3B} are not both alkyl substituted with hydroxy;
R^{4a} and R^{4b} are linked together and form with the nitrogen atom to which they are attached a heterocycle, wherein the heterocycle optionally comprises one further heteroatom selected from N, S and O; and wherein the heterocycle is optionally substituted with one or more substituent selected from alkoxy, alkyl, haloalkyl, alkyloxycarbonyl, cycloalkyl, halo, heteroaryl optionally substituted by one or more alkyl group, hydroxyl, oxo; or the heterocycle is fused with a group selected from aryl and heteroaryl, wherein the aryl or heteroaryl group is optionally substituted with alkyl or alkoxy; with the condition that when R^{4a} and R^{4b} form a piperidine or a morpholine, then the piperidine or morpholine is substituted by at least one of the listed substituents; and
each R⁵ is independently selected from hydrogen and optionally substituted C₁-C₆ alkyl.

The present disclosure thus provides pyrimido[5,4-d]pyrimidine derivatives, which may be useful as ENT inhibitors. In one embodiment, the present disclosure thus provides compounds of formula B:

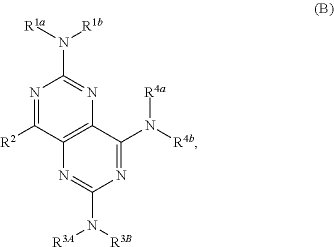

or a pharmaceutically acceptable salt or solvate thereof, wherein
R^{1a} and R^{1b} represent each independently hydrogen, alkyl optionally substituted with one or more of hydroxy, halo, oxo, amino, —NHS(O)₂NR⁵₂, and alkylsulfonyl, arylalkyl wherein the aryl part of the arylalkyl is substituted by one or more alkyl, haloalkyl, halogen, alkoxy or —CO₂H group, heteroarylalkyl, heterocyclylalkyl wherein the heterocyclyl part of the heterocyclylalkyl is optionally substituted by one or more alkyl group, aminocarbonylalkyl, alkylcarbonylaminoalkyl, alkyloxyalkyl, alkylcarbonyloxyalkyl or heterocyclylalkyloxyalkyl; with the condition that R^{1a} and R^{1b} are not both hydrogen;
or R^{1a} and R^{1b} are linked together and form with the nitrogen atom to which they are attached a heterocycle selected from piperidine and piperazine, wherein the heterocycle is optionally substituted with one or more substituent selected from alkyl, alkylcarbonyl, alkyloxycarbonyl, alkylsulfonyl, halo, hydroxy, optionally substituted heteroaryl, and oxo; or the heterocycle is fused with a group selected from aryl and heteroaryl, wherein the aryl or heteroaryl group is optionally substituted with alkyl or alkoxy;
R² represents —NR^{2a}R^{2b} or —OR^{2c}; wherein
   R^{2a} and R^{2b} represent each independently hydrogen, alkyl, alkyloxyalkyl, alkylsulfonylaminoalkyl, arylalkyl wherein the aryl part of the arylalkyl is optionally substituted by one or more of alkoxy, alkylsulfonyl, aminosulfonyl, aminocarbonyl, cyano, halo, haloalkyloxy, optionally substituted heteroaryl, sulfoxide and sulfonylamine groups, aryloxyalkyl, cyanoalkyl, cycloalkyl, heteroarylalkyl wherein the heteroaryl part of the heteroarylalkyl is optionally substituted by one or more of alkyl, halo, haloalkyl and NH₂ groups, heterocyclylalkyl or hydroxyalkyl;
   or R^{2a} and R^{2b} are linked together and form with the nitrogen atom to which they are attached a heterocycle, wherein the heterocycle is optionally substituted with one or more substituent selected from alkoxy, alkyl, alkylcarbonyl, alkylcarbonylamine, alkyloxyalkyl, alkyloxyalkyloxy, alkyloxycarbonyl, alkylsulfonyl, aminocarbonyl, cycloalkyl cyano, halo, haloalkyl, heteroaryl (optionally substituted by one or more of alkyl, cyano and NH₂ groups), hydroxy, hydroxyalkyl and oxo; or two substituents present on a same carbon atom of the heterocycle are linked together and form a spiro heterocycle; or the heterocycle is fused with a group selected from aryl and heteroaryl, wherein the aryl or heteroaryl group is optionally substituted with alkyl or alkoxy; and
   R^{2c} represents arylalkyl wherein the aryl part of the arylalkyl is optionally substituted by one or more of alkoxy, cyano and halo groups; or heteroarylalkyl wherein the heteroaryl part of the heteroarylalkyl is optionally substituted by one or more of alkyl and cyano groups;
R^{3A} and R^{3B} represent each independently alkyl optionally substituted with one or more of hydroxy, halo, oxo, amino, —NHS(O)₂NR⁵₂, and alkylsulfonyl, arylalkyl wherein the aryl part of the arylalkyl is substituted by one or more alkyl, haloalkyl, halogen, alkoxy or —CO₂H group, heteroarylalkyl, heterocyclylalkyl wherein the heterocyclyl part of the heterocyclylalkyl is optionally substituted by one or more alkyl group, aminocarbonylalkyl, alkylcarbonylaminoalkyl, alkyloxyalkyl, alkylcarbonyloxyalkyl or heterocyclylalkyloxyalkyl; with the condition that R^{3A} and R^{3B} are not alkyl substituted with hydroxy;
R^{4a} and R^{4b} are linked together and form with the nitrogen atom to which they are attached a heterocycle, wherein the heterocycle optionally comprises one further heteroatom selected from N, S and O; and wherein the heterocycle is optionally substituted with one or more substituent selected from alkoxy, alkyl, haloalkyl, alkyloxycarbonyl, cycloalkyl, halo, heteroaryl optionally substituted by one or more alkyl group, hydroxyl, oxo; or the heterocycle is fused with a group selected from aryl and heteroaryl, wherein the aryl or heteroaryl group is optionally substituted with alkyl, amine, cyano, or alkoxy; with the condition that when R^{4a} and R^{4b} form a piperidine or a morpholine, then the piperidine or morpholine is substituted by at least one of the listed substituents; and
each R⁵ is independently selected from hydrogen and optionally substituted C₁-C₆ alkyl.

In one embodiment, the present disclosure thus provides compounds of formula I:

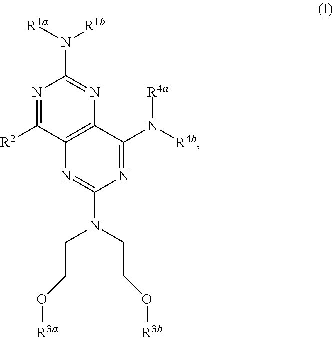

or a pharmaceutically acceptable salt or solvate thereof, wherein
R^{1a} and R^{1b} represent each independently hydrogen, alkyl optionally substituted with one or more of hydroxy, halo, oxo, amino, —NHS(O)₂NR⁵₂, and alkylsulfonyl, arylalkyl wherein the aryl part of the arylalkyl is substituted by one or more alkyl, haloalkyl, halogen, alkoxy or —CO₂H group, heteroarylalkyl, heterocyclylalkyl wherein the heterocyclyl part of the heterocyclylalkyl is optionally substituted by one or more alkyl group, aminocarbonylalkyl, alkylcarbonylaminoalkyl, alkyloxyalkyl, alkylcarbonyloxyalkyl or heterocyclylalkyloxyalkyl; with the condition that R^{1a} and R^{1b} are not both hydrogen;
or R^{1a} and R^{1b} are linked together and form with the nitrogen atom to which they are attached a heterocycle selected from piperidine and piperazine, wherein the heterocycle is optionally substituted with one or more substituent selected from alkyl, alkylcarbonyl, alkyloxycarbonyl, alkylsulfonyl, halo, hydroxy, optionally substituted heteroaryl, and oxo; or the heterocycle is fused with a group selected from aryl and heteroaryl, wherein the aryl or heteroaryl group is optionally substituted with alkyl or alkoxy;
R² represents —NR^{2a}R^{2b} or —OR^{2c}; wherein
   R^{2a} and R^{2b} represent each independently hydrogen, alkyl, alkyloxyalkyl, alkylsulfonylaminoalkyl, arylalkyl wherein the aryl part of the arylalkyl is optionally substituted by one or more of alkoxy, alkylsulfonyl, aminosulfonyl, aminocarbonyl, cyano, halo, haloalkyloxy, optionally substituted heteroaryl, sulfoxide and sulfonylamine groups, aryloxyalkyl, cyanoalkyl, cycloalkyl, heteroarylalkyl wherein the heteroaryl part of the heteroarylalkyl is optionally substituted by one or more of alkyl, halo, haloalkyl and NH₂ groups, heterocyclylalkyl or hydroxyalkyl;
   or R^{2a} and R^{2b} are linked together and form with the nitrogen atom to which they are attached a heterocycle, wherein the heterocycle is optionally substituted with one or more substituent selected from alkoxy, alkyl, alkylcarbonyl, alkylcarbonylamine, alkyloxyalkyl, alkyloxyalkyloxy, alkyloxycarbonyl, alkylsulfonyl, aminocarbonyl, cycloalkyl, cyano, halo, haloalkyl, heteroaryl (optionally substituted by one or more of alkyl, cyano and NH₂ groups), hydroxy, hydroxyalkyl and oxo; or two substituents present on a same carbon atom of the heterocycle are linked together and form a spiro heterocycle; or the heterocycle is fused with a group selected from aryl and heteroaryl, wherein the aryl or heteroaryl group is optionally substituted with alkyl or alkoxy; and
   R^{2c} represents arylalkyl wherein the aryl part of the arylalkyl is optionally substituted by one or more of alkoxy, cyano and halo groups; or heteroarylalkyl wherein the heteroaryl part of the heteroarylalkyl is optionally substituted by one or more of alkyl and cyano groups;
R^{3a} and R^{3b} represent each independently alkyl or alkylcarbonyl;
or R^{3a} is hydrogen and R^{3b} is C₁-C₃ alkyl;
R^{4a} and R^{4b} are linked together and form with the nitrogen atom to which they are attached a heterocycle, wherein the heterocycle optionally comprises one further heteroatom selected from N, S and O; and wherein the heterocycle is optionally substituted with one or more substituent selected from alkoxy, alkyl, haloalkyl, alkyloxycarbonyl, cycloalkyl, halo, heteroaryl optionally substituted by one or more alkyl group, hydroxyl, oxo; or the heterocycle is fused with a group selected from aryl and heteroaryl, wherein the aryl or heteroaryl group is optionally substituted with alkyl, amine, cyano, or alkoxy; with the condition that when R^{4a} and R^{4b} form a piperidine or a morpholine, then the piperidine or morpholine is substituted by at least one of the listed substituents; and
each R⁵ is independently selected from hydrogen and optionally substituted C₁-C₆ alkyl.

In one embodiment, in formula I:
R^{1a} and R^{1b} represent each independently hydrogen, alkyl, arylalkyl wherein the aryl part of the arylalkyl is substituted by one or more alkoxy group, heteroarylalkyl, heterocyclylalkyl wherein the heterocyclyl part of the heterocyclylalkyl is optionally substituted by one or more alkyl group, aminocarbonylalkyl, hydroxyalkyl, alkyloxyalkyl, alkylcarbonyloxyalkyl or heterocyclylalkyloxyalkyl; with the condition that R^{1a} and R^{1b} are not both hydrogen;
or R^{1a} and R^{1b} are linked together and form with the nitrogen atom to which they are attached a heterocycle selected from piperidine and piperazine, wherein the heterocycle is optionally substituted with one or more substituent selected from alkyl, alkylcarbonyl, alkyloxycarbonyl, alkylsulfonyl, halo, hydroxy and oxo;
R² represents —NR^{2a}R^{2b} or —OR^{2c}; wherein
   R^{2a} and R^{2b} represent each independently hydrogen, alkyl, alkyloxyalkyl, alkylsulfonylaminoalkyl, arylalkyl wherein the aryl part of the arylalkyl is optionally substituted by one or more of alkoxy, alkylsulfonyl, aminosulfonyl, aminocarbonyl, cyano, halo, haloalkyloxy, heteroaryl, sulfoxide and sulfonylamine groups, aryloxyalkyl, cyanoalkyl, cycloalkyl, heteroarylalkyl wherein the heteroaryl part of the heteroarylalkyl is optionally substituted by one or more of alkyl, halo, haloalkyl and NH₂ groups, heterocyclylalkyl or hydroxyalkyl;
   or R^{2a} and R^{2b} are linked together and form with the nitrogen atom to which they are attached a heterocycle, wherein the heterocycle is optionally substituted with one or more substituent selected from alkoxy, alkyl, alkylcarbonyl, alkylcarbonylamine, alkyloxyalkyl, alkyloxyalkyloxy, alkyloxycarbonyl, alkylsulfonyl, aminocarbonyl, cyano, halo, haloalkyl, heteroaryl (optionally substituted by one or more of alkyl, cyano and NH₂ groups), hydroxy, hydroxyalkyl and oxo; or two substituents present on a same carbon atom of the heterocycle are linked together and form a spiro heterocycle; or the heterocycle is fused with a group selected from aryl and heteroaryl, wherein the aryl or heteroaryl group is optionally substituted with alkyl or alkoxy; and
   R^{2c} represents arylalkyl wherein the aryl part of the arylalkyl is optionally substituted by one or more of alkoxy, cyano and halo groups; or heteroarylalkyl wherein the heteroaryl part of the heteroarylalkyl is optionally substituted by one or more of alkyl and cyano groups;
R^{3a} and R^{3b} represent each independently alkyl or alkylcarbonyl; and
R^{4a} and R^{4b} are linked together and form with the nitrogen atom to which they are attached a heterocycle, wherein the heterocycle optionally comprises one further heteroatom selected from N, S and O; and wherein the heterocycle is optionally substituted with one or more substituent selected from alkoxy, alkyl, haloalkyl, alkyloxycarbonyl, halo, heteroaryl optionally substituted by one or more alkyl group, hydroxyl, oxo; or the heterocycle is fused with a group selected from aryl and heteroaryl, wherein the aryl or heteroaryl group is optionally substituted with alkyl or alkoxy; with the condition that when R^{4a} and R^{4b} form a piperidine or a morpholine, then the piperidine or morpholine is substituted by at least one of the listed substituents.

In one embodiment, in formula I:
R^{1a} and R^{1b} represent each independently hydrogen, alkyl, arylalkyl wherein the aryl part of the arylalkyl is substituted by one or more alkoxy group, heteroarylalkyl, heterocyclylalkyl wherein the heterocyclyl part of the heterocyclylalkyl is optionally substituted by one or more alkyl group, hydroxyalkyl, alkyloxyalkyl, alkylcarbonyloxyalkyl or heterocyclylalkyloxyalkyl; with the condition that R^{1a} and R^{1b} are not both hydrogen;
or R^{1a} and R^{1b} are linked together and form with the nitrogen atom to which they are attached a heterocycle selected from piperidine and piperazine, wherein the heterocycle is optionally substituted with one or more substituent selected from alkyl, alkylcarbonyl, alkyloxycarbonyl, alkylsulfonyl, halo, hydroxy and oxo;
R² represents —NR^{2a}R^{2b} or —OR^{2c}; wherein
   R^{2a} and R^{2b} represent each independently hydrogen, alkyl, alkyloxyalkyl, alkylsulfonylaminoalkyl, arylalkyl wherein the aryl part of the arylalkyl is optionally substituted by one or more of alkoxy, alkylsulfonyl, cyano, halo, haloalkyloxy, heteroaryl, sulfoxide and sulfonylamine groups, aryloxyalkyl, cyanoalkyl, cycloalkyl, heteroarylalkyl wherein the heteroaryl part of the heteroarylalkyl is optionally substituted by one or more of alkyl, halo, haloalkyl and NH₂ groups, heterocyclylalkyl or hydroxyalkyl;
   or R^{2a} and R^{2b} are linked together and form with the nitrogen atom to which they are attached a heterocycle, wherein the heterocycle is optionally substituted with one or more substituent selected from alkoxy, alkyl, alkylcarbonyl, alkylcarbonylamine, alkyloxyalkyl, alkyloxycarbonyl, alkylsulfonyl, aminocarbonyl, cyano, halo, haloalkyl, heteroaryl (optionally substituted by one or more of alkyl, cyano and NH₂ groups), hydroxy, hydroxyalkyl and oxo; or two substituents present on a same carbon atom of the heterocycle are linked together and form a spiro heterocycle; or the heterocycle is fused with a group selected from aryl and heteroaryl, wherein the aryl or heteroaryl group is optionally substituted with alkyl or alkoxy; and
   R^{2c} represents arylalkyl wherein the aryl part of the arylalkyl is optionally substituted by one or more of alkoxy, cyano and halo groups; or heteroarylalkyl wherein the heteroaryl part of the heteroarylalkyl is optionally substituted by one or more of alkyl and cyano groups;
R^{3a} and R^{3b} represent each independently alkyl or alkylcarbonyl; and
R^{4a} and R^{4b} are linked together and form with the nitrogen atom to which they are attached a heterocycle, wherein the heterocycle optionally comprises one further heteroatom selected from N, S and O; and wherein the heterocycle is optionally substituted with one or more substituent selected from alkoxy, alkyl, haloalkyl, alkyloxycarbonyl, halo, heteroaryl optionally substituted by one or more alkyl group, hydroxyl, oxo; or the heterocycle is fused with a group selected from aryl and heteroaryl, wherein the aryl or heteroaryl group is optionally substituted with alkyl or alkoxy; with the condition that when R^{4a} and R^{4b} form a piperidine or a morpholine, then the piperidine or morpholine is substituted by at least one of the listed substituents.

In one embodiment, compounds of formula I are of formula Ia:

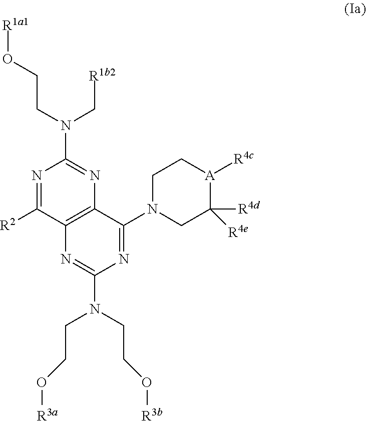

or a pharmaceutically acceptable salt or solvate thereof, wherein
R^{1a1} represents hydrogen, alkyl, alkylcarbonyl or heterocyclylalkyl; preferably R^{1a1} represents hydrogen, methyl, methylcarbonyl, morpholinoethyl, morpholinopropyl or benzosuccinimidylethyl;
R^{1b2} represents alkylcarbonyloxyalkyl, alkyloxyalkyl, arylalkyl wherein the aryl part of the arylalkyl is substituted by one or more alkoxy group, heterocyclylalkyloxyalkyl or hydroxyalkyl; preferably R^{1b2} represents methylcarbonyloxymethyl, methoxymethyl, trimethoxyphenyl, morpholinoethoxymethyl, morpholinopropyloxymethyl, benzosuccinimidylethoxymethyl or hydroxymethyl;
R², R^{3a} and R^{3b} are as defined as in formula I;
A represents CH, N or SO₂;
R^{4c} is absent or represents hydrogen, alkoxy, alkyl, alkyloxycarbonyl, halo or heteroaryl optionally substituted by one or more alkyl group; with the condition that when A is CH, then R^{4c} is not hydrogen; preferably R^{4c} is absent or represents hydrogen, methoxy, methyl, ethyloxycarbonyl, fluorine, thiazole, methyl-triazole or methyl-oxadiazole, with the condition that when A is CH, then R^{4c} is not hydrogen; and
R^{4d} and R^{4e} are both hydrogen atoms or form together an oxo group.

In one embodiment, compounds of formula Ia are of formula Ia1:

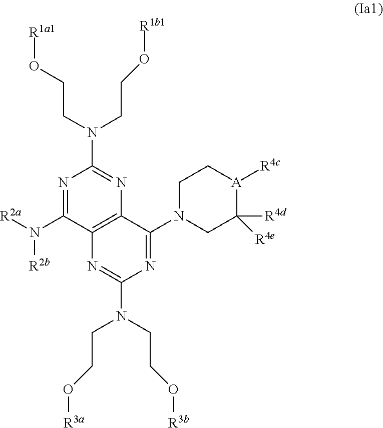

or a pharmaceutically acceptable salt or solvate thereof, wherein
R^{1a1} and R^{1b1} represent each independently hydrogen, alkyl, alkylcarbonyl or heterocyclylalkyl; preferably R^{1a1} and R^{1b1} represent each independently alkyl, alkylcarbonyl or heterocyclylalkyl; more preferably R^{1a1} and R^{1b1} represent each independently methyl, methylcarbonyl, morpholinoethyl, morpholinopropyl or benzosuccinimidylethyl; and
R^{2a}, R^{2b}, R^{3a}, R^{3b}, A, R^{4c}, R^{4d} and R^{4e} are defined above.

R^{1a} and R^{1b}
In some embodiments, R^{1a} and R^{1b} represent each independently hydrogen, alkyl optionally substituted with one or more of hydroxy, halo, oxo, amino, —NHS(O)₂NR⁵₂, and alkylsulfonyl, arylalkyl wherein the aryl part of the arylalkyl is substituted by one or more alkyl, haloalkyl, halogen, alkoxy or —CO₂H group, heteroarylalkyl, heterocyclylalkyl wherein the heterocyclyl part of the heterocyclylalkyl is optionally substituted by one or more alkyl group, aminocarbonylalkyl, alkylcarbonylaminoalkyl, alkyloxyalkyl, alkylcarbonyloxyalkyl or heterocyclylalkyloxyalkyl; with the condition that R^{1a} and R^{1b} are not both hydrogen;
or R^{1a} and R^{1b} are linked together and form with the nitrogen atom to which they are attached a heterocycle selected from piperidine and piperazine, wherein the heterocycle is optionally substituted with one or more substituent selected from alkyl, alkylcarbonyl, alkyloxycarbonyl, alkylsulfonyl, halo, hydroxy, optionally substituted heteroaryl, and oxo; or the heterocycle is fused with a group selected from aryl and heteroaryl, wherein the aryl or heteroaryl group is optionally substituted with alkyl or alkoxy;
wherein each R⁵ is independently selected from hydrogen and optionally substituted C₁-C₆ alkyl.

In some embodiments, each of R^{1a} and R^{1b} are independently selected from the group consisting of:

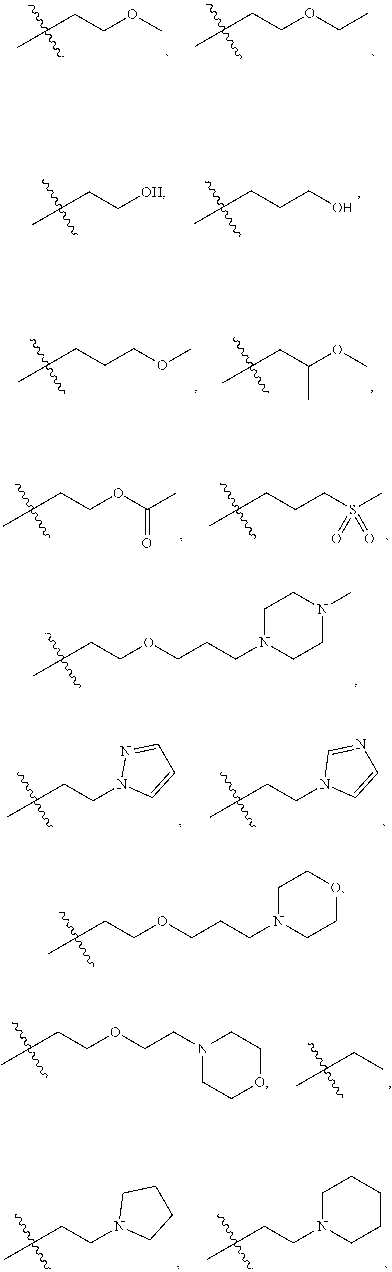

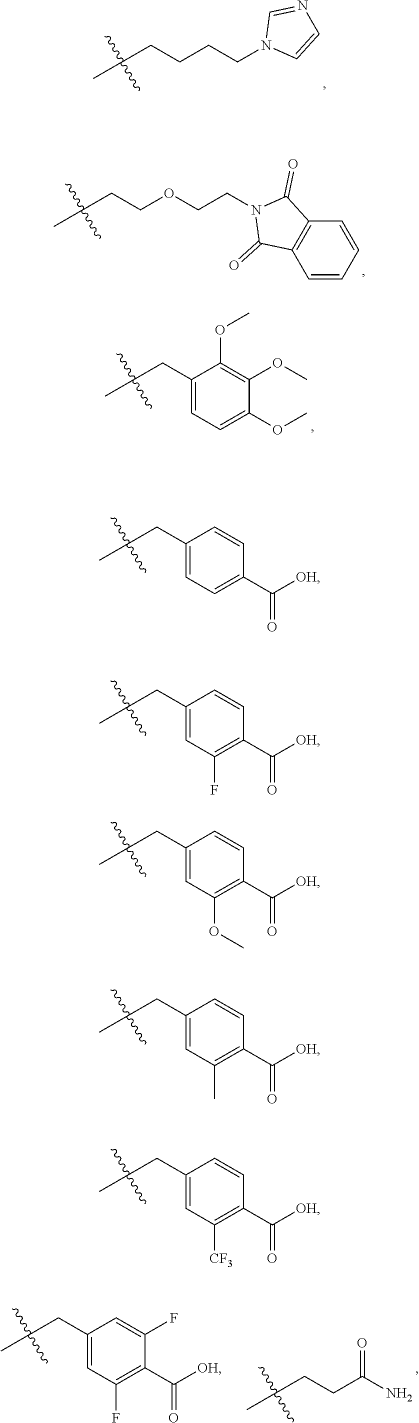

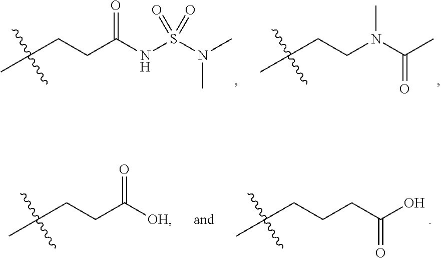

In some embodiments, R^{1a} and R^{1b} are linked together and form, with the nitrogen atom to which they are attached, a heterocycle selected from the group consisting of:

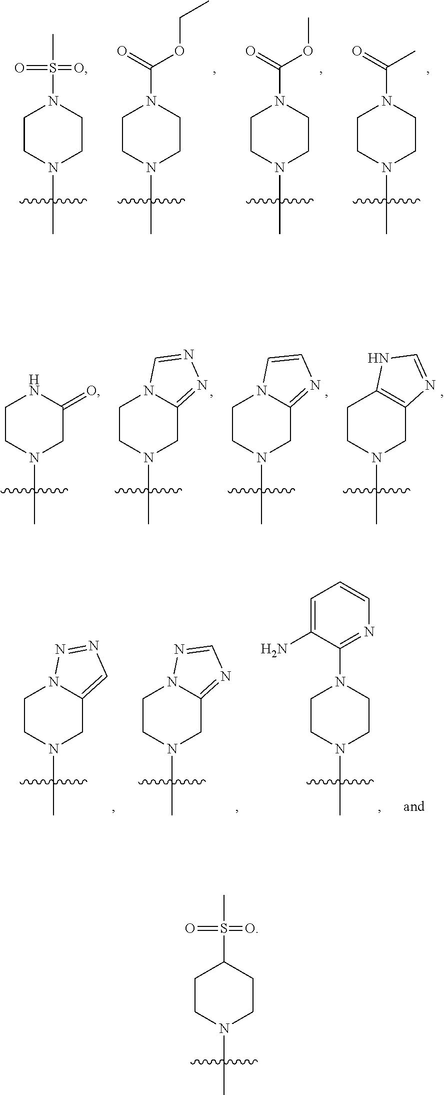

R²

In some embodiments, R² represents —NR^{2a}R^{2b} or —OR^{2c}; wherein R^{2a} and R^{2b} represent each independently hydrogen, alkyl, alkyloxyalkyl, alkylsulfonylaminoalkyl, arylalkyl wherein the aryl part of the arylalkyl is optionally substituted by one or more of alkoxy, alkylsulfonyl, aminosulfonyl, aminocarbonyl, cyano, halo, haloalkyloxy, optionally substituted heteroaryl, sulfoxide and sulfonylamine groups, aryloxyalkyl, cyanoalkyl, cycloalkyl, heteroarylalkyl wherein the heteroaryl part of the heteroarylalkyl is optionally substituted by one or more of alkyl, halo, haloalkyl and NH₂ groups, heterocyclylalkyl or hydroxyalkyl; or R^{2a} and R^{2b} are linked together and form with the nitrogen atom to which they are attached a heterocycle, wherein the heterocycle is optionally substituted with one or more substituent selected from alkoxy, alkyl, alkylcarbonyl, alkylcarbonylamine, alkyloxyalkyl, alkyloxyalkyloxy, alkyloxycarbonyl, alkylsulfonyl, aminocarbonyl, cycloalkyl, cyano, halo, haloalkyl, heteroaryl (optionally substituted by one or more of alkyl, cyano and NH₂ groups), hydroxy, hydroxyalkyl and oxo; or two substituents present on a same carbon atom of the heterocycle are linked together and form a spiro heterocycle; or the heterocycle is fused with a group selected from aryl and heteroaryl, wherein the aryl or heteroaryl group is optionally substituted with alkyl or alkoxy; and R^{2c} represents arylalkyl wherein the aryl part of the arylalkyl is optionally substituted by one or more of alkoxy, cyano and halo groups; or heteroarylalkyl wherein the heteroaryl part of the heteroarylalkyl is optionally substituted by one or more of alkyl and cyano groups.

In some embodiments, R² is piperidine or piperazine optionally substituted with one or more substituent selected from alkoxy, alkyl, alkylcarbonyl, alkylcarbonylamine, alkyloxyalkyl, alkyloxyalkyloxy, alkyloxycarbonyl, alkylsulfonyl, aminocarbonyl, cycloalkyl cyano, halo, haloalkyl, heteroaryl (optionally substituted by one or more of alkyl, cyano and NH₂ groups), hydroxy, hydroxyalkyl and oxo.

In some embodiments, R² is piperidine optionally substituted with one or more substituent selected from alkoxy, alkyl, alkylcarbonyl, alkylcarbonylamine, alkyloxyalkyl, alkyloxyalkyloxy, alkyloxycarbonyl, alkylsulfonyl, aminocarbonyl, cycloalkyl cyano, halo, haloalkyl, heteroaryl (optionally substituted by one or more of alkyl, cyano and NH₂ groups), hydroxy, hydroxyalkyl and oxo.

In some embodiments, R² is piperazine optionally substituted with one or more substituent selected from alkoxy, alkyl, alkylcarbonyl, alkylcarbonylamine, alkyloxyalkyl, alkyloxyalkyloxy, alkyloxycarbonyl, alkylsulfonyl, aminocarbonyl, cycloalkyl cyano, halo, haloalkyl, heteroaryl (optionally substituted by one or more of alkyl, cyano and NH₂ groups), hydroxy, hydroxyalkyl and oxo.

In some embodiments, R² is selected from the group consisting of:

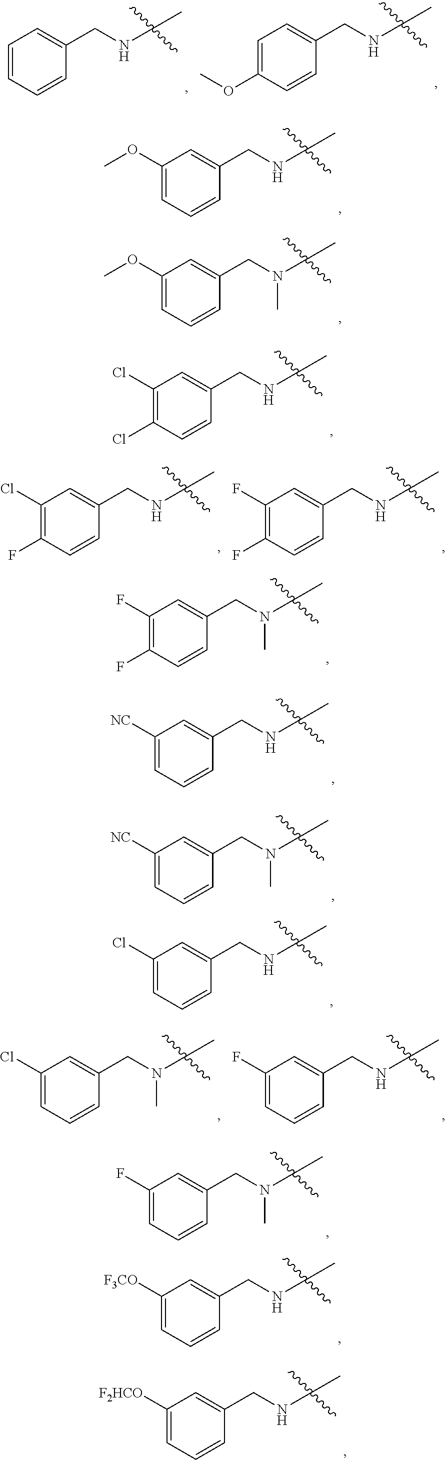

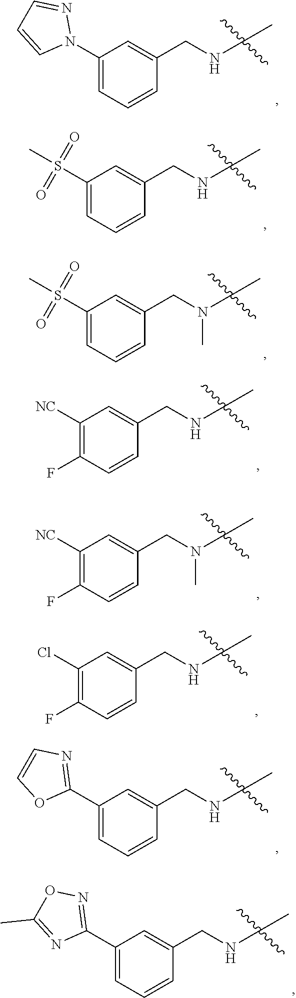

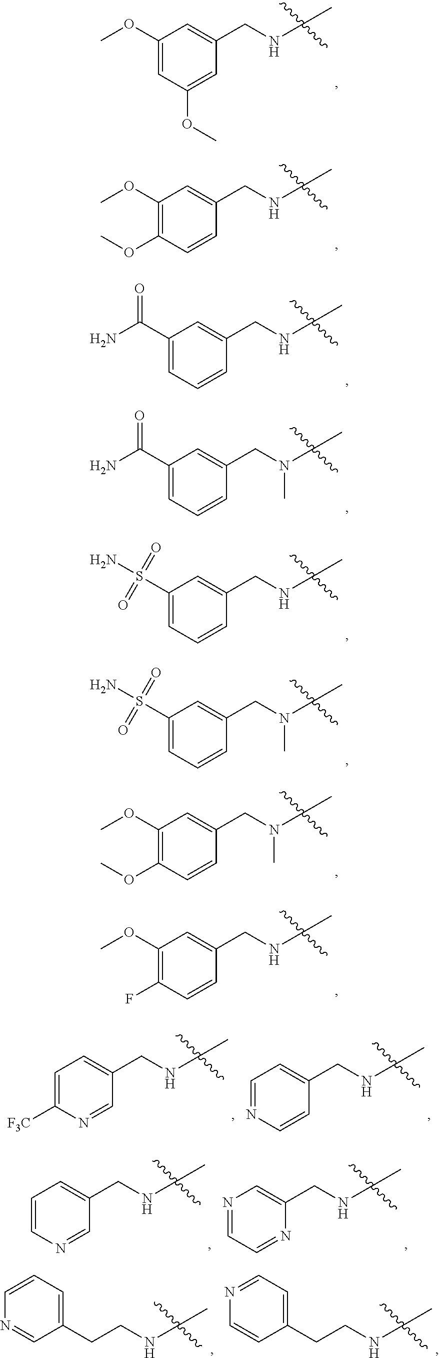

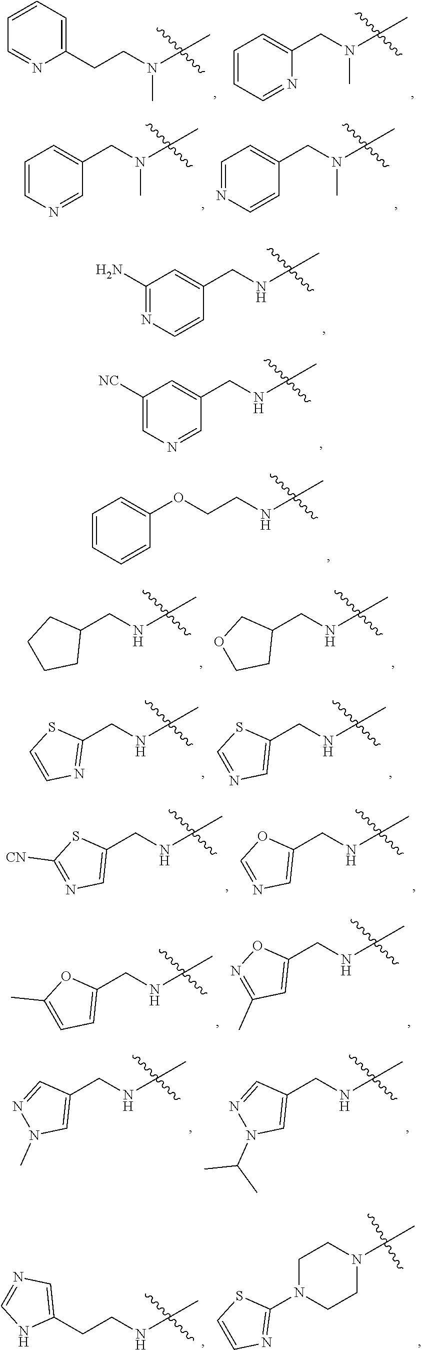

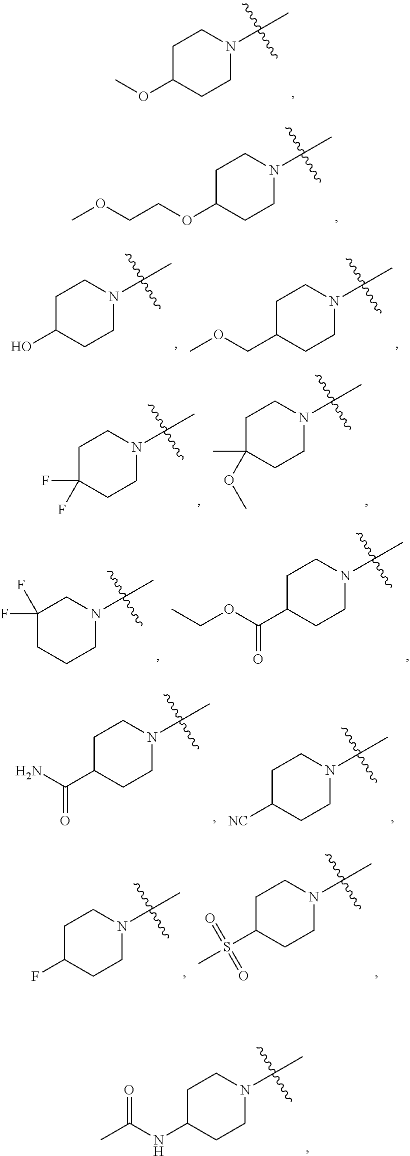

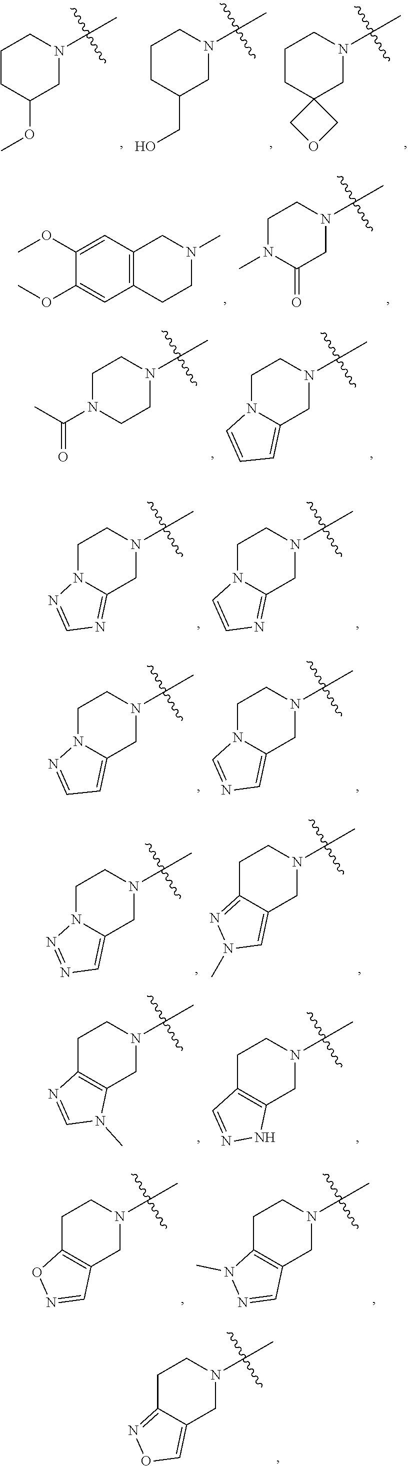

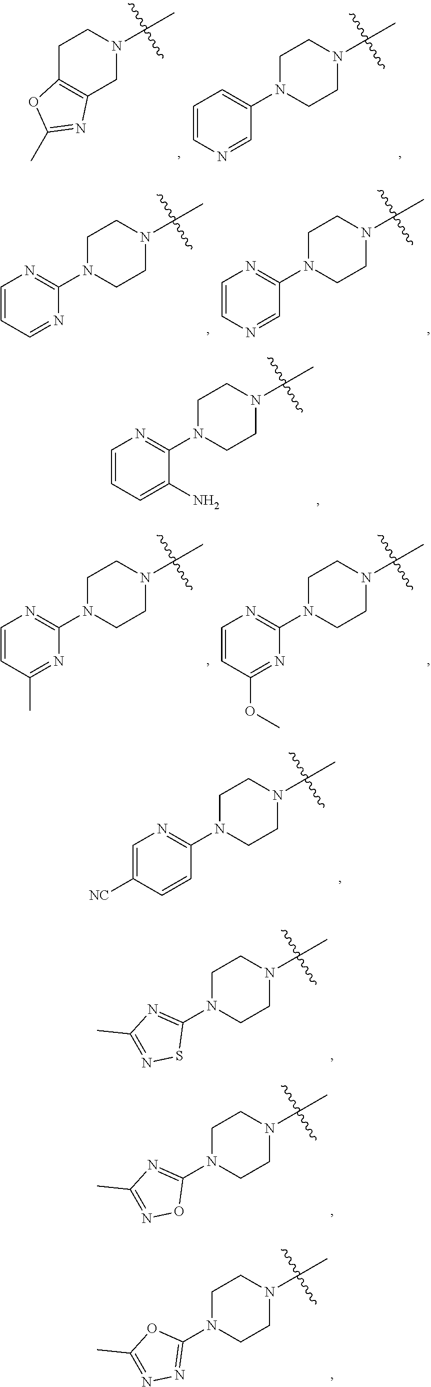

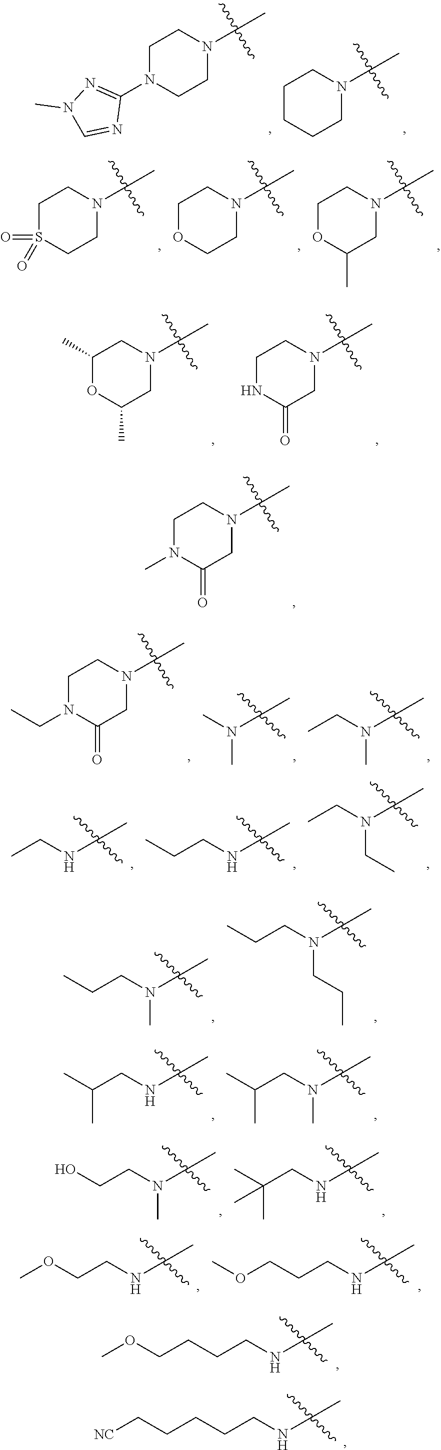

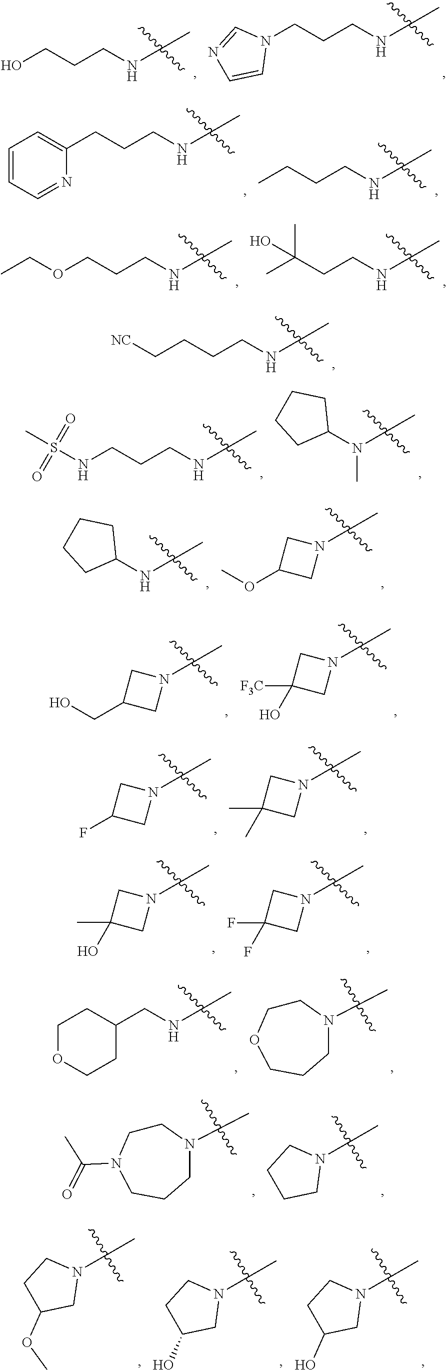

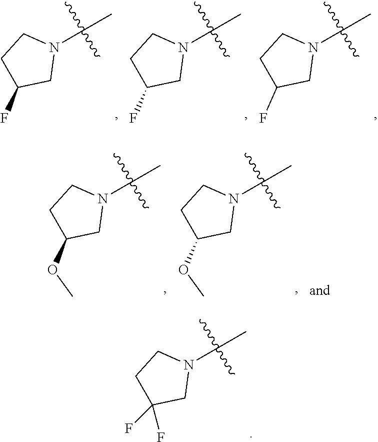

In some embodiments, R² is

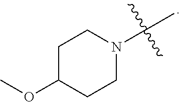 [SMILES: *N1CCC(OC)CC1]

In some embodiments, R² is

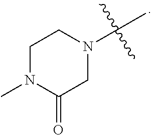 [SMILES: *N1CCN(C)C(=O)C1]

R^{4a} and R^{4b}

In some embodiments, R^{4a} and R^{4b} are linked together and form with the nitrogen atom to which they are attached a heterocycle, wherein the heterocycle optionally comprises one further heteroatom selected from N, S and O; and wherein the heterocycle is optionally substituted with one or more substituent selected from alkoxy, alkyl, haloalkyl, alkyloxycarbonyl, cycloalkyl, halo, heteroaryl optionally substituted by one or more alkyl group, hydroxyl, oxo; or the heterocycle is fused with a group selected from aryl and heteroaryl, wherein the aryl or heteroaryl group is optionally substituted with alkyl, amine, cyano or alkoxy; with the condition that when R^{4a} and R^{4b} form a piperidine or a morpholine, then the piperidine or morpholine is substituted by at least one of the listed substituents.

In some embodiments, R^{4a} and R^{4b} are linked together and form with the nitrogen atom to which they are attached piperidine substituted with one or more substituent selected from alkoxy, alkyl, haloalkyl, alkyloxycarbonyl, cycloalkyl, halo, heteroaryl optionally substituted by one or more alkyl group, hydroxyl, oxo; or the piperidine is fused with a group selected from aryl and heteroaryl, wherein the aryl or heteroaryl group is optionally substituted with alkyl, amine, cyano or alkoxy.

In some embodiments, R^{4a} and R^{4b} are linked together and form with the nitrogen atom to which they are attached piperazine optionally substituted with one or more substituent selected from alkoxy, alkyl, haloalkyl, alkyloxycarbonyl, cycloalkyl, halo, heteroaryl optionally substituted by one or more alkyl group, hydroxyl, oxo; or the piperazine is fused with a group selected from aryl and heteroaryl, wherein the aryl or heteroaryl group is optionally substituted with alkyl, amine, cyano or alkoxy.

In some embodiments, R^{4a} and R^{4b} are linked together and form with the nitrogen atom to which they are attached a heterocycle selected from the group consisting of:

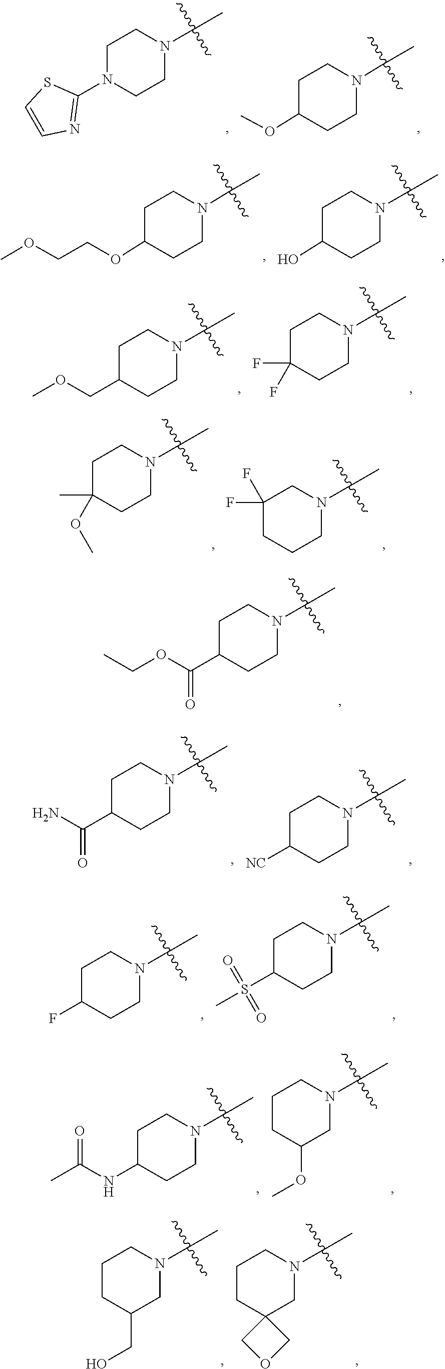

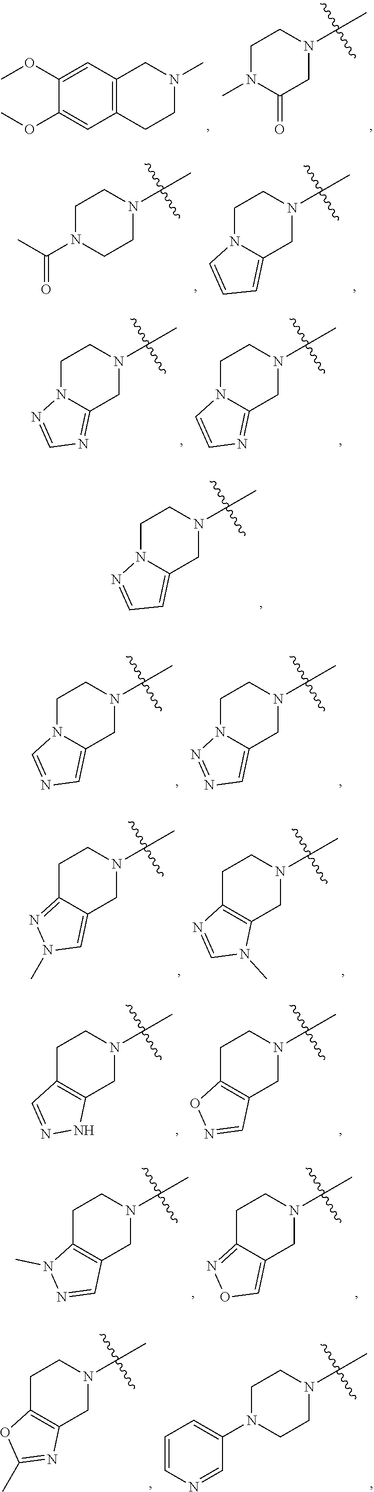

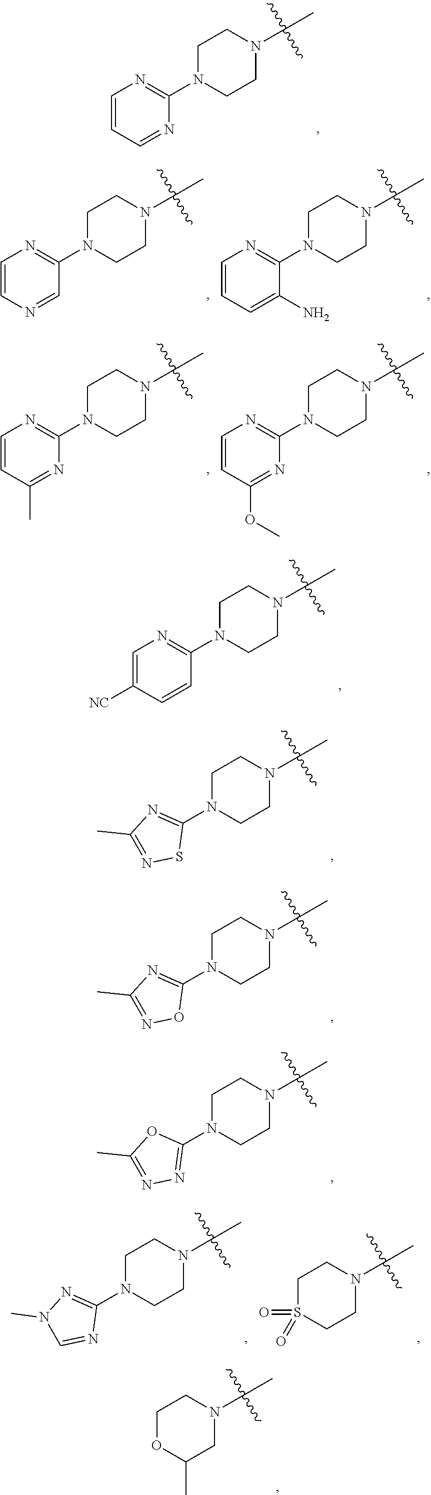

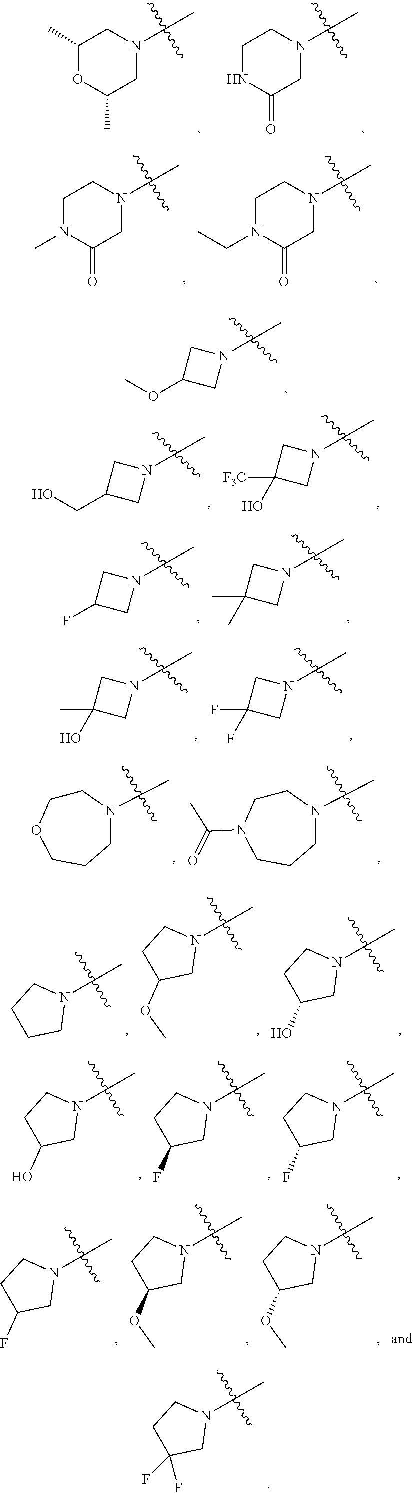

In some embodiments, R^{4a} and R^{4b} are linked together and form

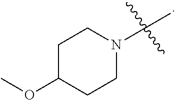

In some embodiments, R^{4a} and R^{4b} are linked together and form

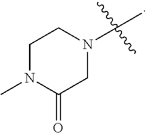

Particularly preferred compound structures of formula I of the invention are those listed in Table 1 hereafter.

TABLE 1

| | Compound Structures | Chemical name | MW |
|---|---|---|---|
| 1 | 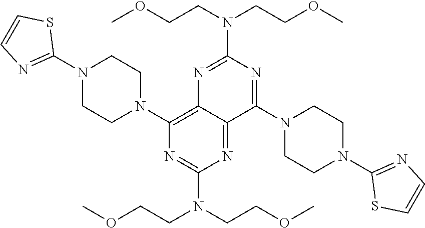 [SMILES: COCCN(CCOC)c1nc(N2CCN(c3nccs3)CC2)c2nc(N(CCOC)CCOC)nc(N3CCN(c4nccs4)CC3)c2n1] | N2,N2,N6,N6-tetrakis(2-methoxyethyl)-4,8- bis(4-(thiazol-2-yl)piperazin-1- yl)pyrimido[5,4-d]pyrimidine-2,6-diamine | 728.932 |
| 2 | 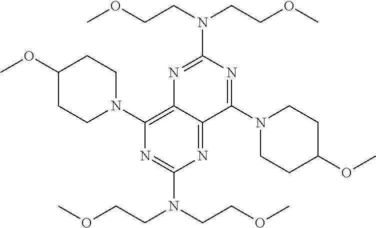 | N2,N2,N6,N6-tetrakis(2-methoxyethyl)-4,8- bis(4-methoxypiperidin-1-yl)pyrimido[5,4- d]pyrimidine-2,6-diamine | 620.784 |
| 3 | 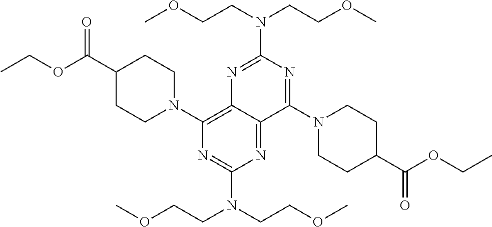 [SMILES: CCOC(=O)C1CCN(c2nc(N(CCOC)CCOC)nc3c(N4CCC(C(=O)OCC)CC4)nc(N(CCOC)CCOC)nc23)CC1] | diethyl 1,1′-(2,6-bis(bis(2- methoxyethyl)amino)pyrimido[5,4- d]pyrimidine-4,8-diyl)bis(piperidine-4- carboxylate) | 704.857 |
| 4 | 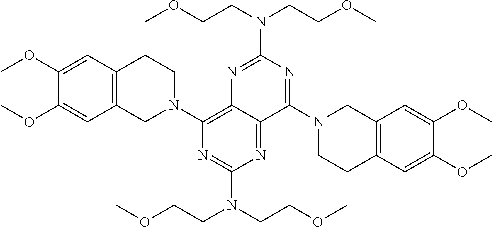 | 4,8-bis(6,7-dimethoxy-3,4-dihydroisoquinolin- 2(1H)-yl)-N2,N2,N6,N6-tetrakis(2- methoxyethyl)pyrimido[5,4-d]pyrimidine-2,6- diamine | 776.921 |
| 5 | 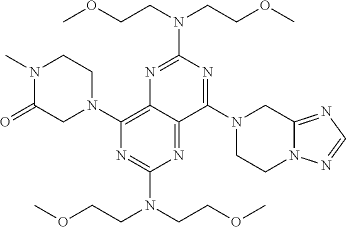 [SMILES: COCCN(CCOC)c1nc(N2CCn3ncnc3C2)c2nc(N(CCOC)CCOC)nc(N3CCN(C)C(=O)C3)c2n1] | 4-(2,6-bis(bis(2-methoxyethyl)amino)-8-(5,6- dihydro-[1,2,4]triazolo[1,5-a]pyrazin-7(8H)- yl)pyrimido[5,4-d]pyrimidin-4-yl)-1- methylpiperazin-2-one | 628.726 |
| 6 | 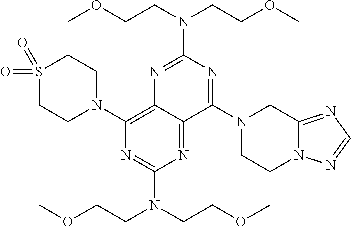 [SMILES: COCCN(CCOC)c1nc(N2CCn3ncnc3C2)c2nc(N(CCOC)CCOC)nc(N3CCS(=O)(=O)CC3)c2n1] | 4-(2,6-bis(bis(2-methoxyethyl)amino)-8-(5,6- dihydro-[1,2,4]triazolo[1,5-a]pyrazin-7(8H)- yl)pyrimido[5,4-d]pyrimidin-4- yl)thiomorpholine 1,1-dioxide | 649.765 |
| 7 | 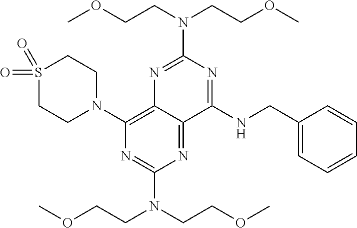 [SMILES: COCCN(CCOC)c1nc(N2CCS(=O)(=O)CC2)c2nc(N(CCOC)CCOC)nc(NCc3ccccc3)c2n1] | 4-(8-(benzylamino)-2,6-bis(bis(2- methoxyethyl)amino)pyrimido[5,4- d]pyrimidin-4-yl)thiomorpholine 1,1-dioxide | 632.775 |
| 8 | 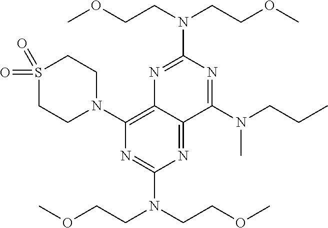 [SMILES: CCCN(C)c1nc(N(CCOC)CCOC)nc2c(N3CCS(=O)(=O)CC3)nc(N(CCOC)CCOC)nc12] | 4-(2,6-bis(bis(2-methoxyethyl)amino)-8- (methyl(propyl)amino)pyrimido[5,4- d]pyrimidin-4-yl)thiomorpholine 1,1-dioxide | 598.758 |
| 9 | 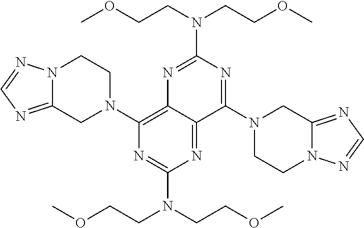 [SMILES: COCCN(CCOC)c1nc(N2CCn3ncnc3C2)c2nc(N(CCOC)CCOC)nc(N3CCn4ncnc4C3)c2n1] | 4,8-bis(5,6-dihydro-[1,2,4]triazolo[1,5- a]pyrazin-7(8H)-yl)-N2,N2,N6,N6-tetrakis(2- methoxyethyl)pyrimido[5,4-d]pyrimidine-2,6- diamine | 638.724 |
| 10 | 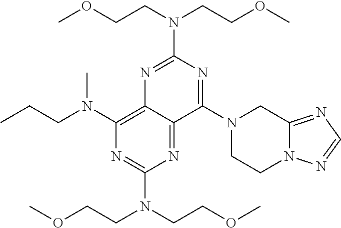 [SMILES: CCCN(C)c1nc(N(CCOC)CCOC)nc2c(N3CCn4ncnc4C3)nc(N(CCOC)CCOC)nc12] | 8-(5,6-dihydro-[1,2,4]triazolo[1,5-a]pyrazin- 7(8H)-yl)-N2,N2,N6,N6-tetrakis(2- methoxyethyl)-N4-methyl-N4- propylpyrimido[5,4-d]pyrimidine-2,4,6- triamine | 587.718 |
| 11 | 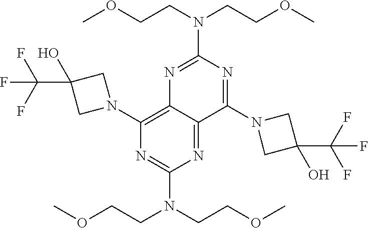 [SMILES: COCCN(CCOC)c1nc(N2CC(O)(C(F)(F)F)C2)c2nc(N(CCOC)CCOC)nc(N3CC(O)(C(F)(F)F)C3)c2n1] | 1,1′-(2,6-bis(bis(2- methoxyethyl)amino)pyrimido[5,4- d]pyrimidine-4,8-diyl)bis(3- (trifluoromethyl)azetidin-3-ol) | 672.62 |
| 12 | 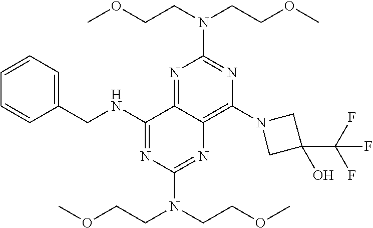 [SMILES: COCCN(CCOC)c1nc(N2CC(O)(C(F)(F)F)C2)c2nc(N(CCOC)CCOC)nc(NCc3ccccc3)c2n1] | 1-(8-(benzylamino)-2,6-bis(bis(2- methoxyethyl)amino)pyrimido[5,4- d]pyrimidin-4-yl)-3-(trifluoromethyl)azetidin- 3-ol | 638.682 |
| 13 | 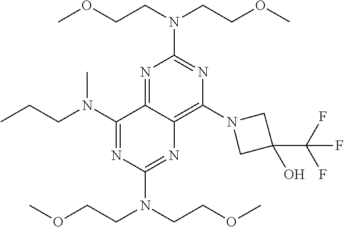 [SMILES: CCCN(C)c1nc(N(CCOC)CCOC)nc2c(N3CC(O)(C(F)(F)F)C3)nc(N(CCOC)CCOC)nc12] | 1-(2,6-bis(bis(2-methoxyethyl)amino)-8- (methyl(propyl)amino)pyrimido[5,4- d]pyrimidin-4-yl)-3-(trifluoromethyl)azetidin- 3-ol | 604.665 |
| 14 | 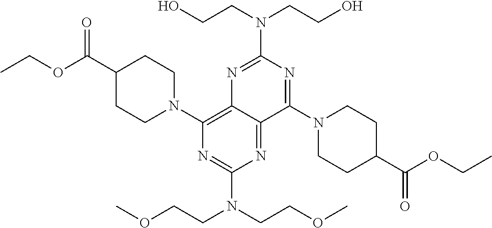 [SMILES: CCOC(=O)C1CCN(c2nc(N(CCOC)CCOC)nc3c(N4CCC(C(=O)OCC)CC4)nc(N(CCO)CCO)nc23)CC1] | diethyl 1,1′-(2-(bis(2-hydroxyethyl)amino)-6- (bis(2-methoxyethyl)amino)pyrimido[5,4- d]pyrimidine-4,8-diyl)bis(piperidine-4- carboxylate) | 676.804 |
| 15 | 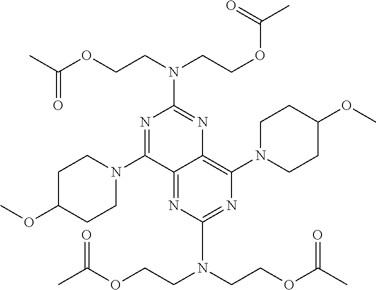 | ((4,8-bis(4-methoxypiperidin-1- yl)pyrimido[5,4-d]pyrimidine-2,6- diyl)bis(azanetriyl))tetrakis(ethane-2,1- diyl)tetraacetate | 732.824 |
| 16 | 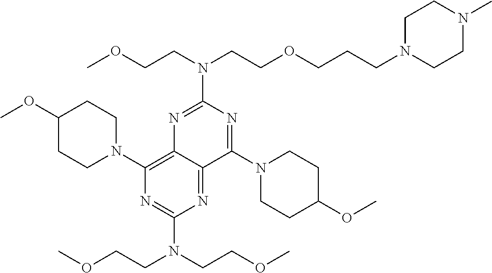 | N2,N2,N6-tris(2-methoxyethyl)-4,8-bis(4- methoxypiperidin-1-yl)-N6-(2-(3-(4- methylpiperazin-1- yl)propoxy)ethyl)pyrimido[5,4-d]pyrimidine- 2,6-diamine | 746.983 |
| 19 | 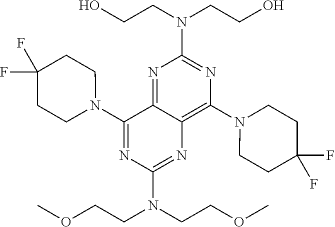 [SMILES: COCCN(CCOC)c1nc(N2CCC(F)(F)CC2)c2nc(N(CCO)CCO)nc(N3CCC(F)(F)CC3)c2n1] | 2,2′-((6-(bis(2-methoxyethyl)amino)-4,8- bis(4,4-difluoropiperidin-1-yl)pyrimido[5,4- d]pyrimidin-2-yl)azanediyl)diethanol | 604.641 |
| 27 | 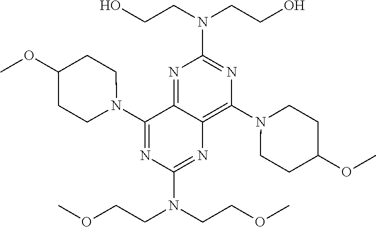 | 2,2′-((6-(bis(2-methoxyethyl)amino)-4,8-bis(4- methoxypiperidin-1-yl)pyrimido[5,4- d]pyrimidin-2-yl)azanediyl)diethanol | 592.731 |
| 30 | 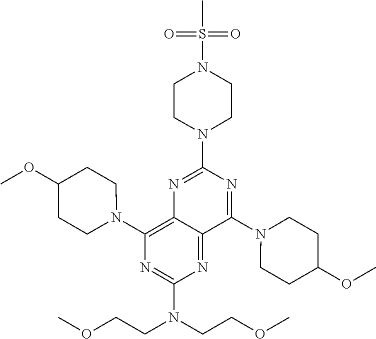 | N,N-bis(2-methoxyethyl)-4,8-bis(4- methoxypiperidin-1-yl)-6-(4- (methylsulfonyl)piperazin-1-yl)pyrimido[5,4- d]pyrimidin-2-amine | 651.821 |
| 31 | 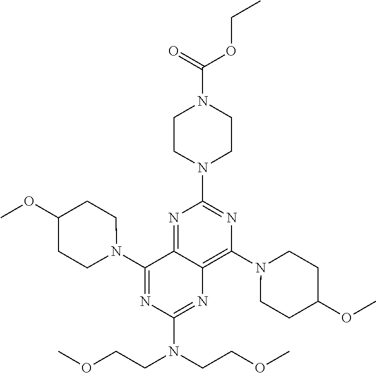 | ethyl 4-(6-(bis(2-methoxyethyl)amino)-4,8- bis(4-methoxypiperidin-1-yl)pyrimido[5,4- d]pyrimidin-2-yl)piperazine-1-carboxylate | 645.793 |
| 32 | 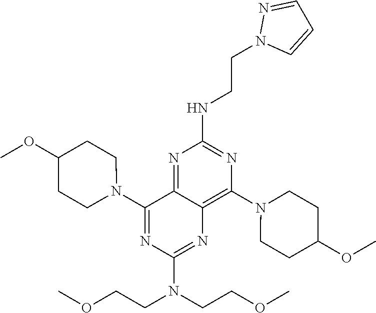 | N2-(2-(1H-pyrazol-1-yl)ethyl)-N6,N6-bis(2- methoxyethyl)-4,8-bis(4-methoxypiperidin-1- yl)pyrimido[5,4-d]pyrimidine-2,6-diamine | 598.74 |
| 33 | 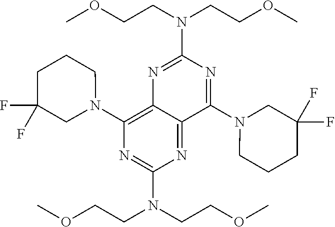 [SMILES: COCCN(CCOC)c1nc(N2CCCC(F)(F)C2)c2nc(N(CCOC)CCOC)nc(N3CCCC(F)(F)C3)c2n1] | 4,8-bis(3,3-difluoropiperidin-1-yl)- N2,N2,N6,N6-tetrakis(2- methoxyethyl)pyrimido[5,4-d]pyrimidine-2,6- diamine | 632.694 |
| 34 | 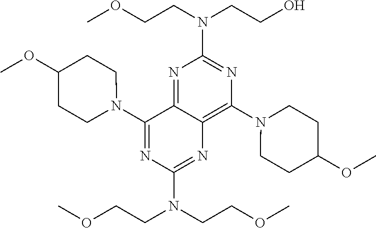 | 2-((6-(bis(2-methoxyethyl)amino)-4,8-bis(4- methoxypiperidin-1-yl)pyrimido[5,4- d]pyrimidin-2-yl)(2- methoxyethyl)amino)ethanol | 606.757 |
| 37 | 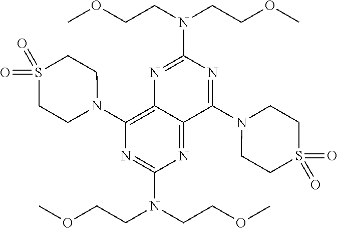 [SMILES: COCCN(CCOC)c1nc(N2CCS(=O)(=O)CC2)c2nc(N(CCOC)CCOC)nc(N3CCS(=O)(=O)CC3)c2n1] | 4,4′-(2,6-bis(bis(2- methoxyethyl)amino)pyrimido[5,4- d]pyrimidine-4,8-diyl)bis(thiomorpholine 1,1- dioxide) | 660.806 |
| 38 | 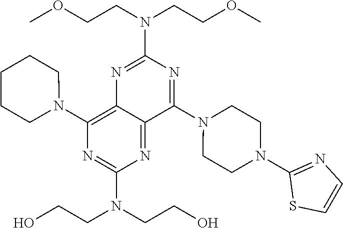 [SMILES: COCCN(CCOC)c1nc(N2CCN(c3nccs3)CC2)c2nc(N(CCO)CCO)nc(N3CCCCC3)c2n1] | 2,2′-((6-(bis(2-methoxyethyl)amino)-4- (piperidin-1-yl)-8-(4-(thiazol-2-yl)piperazin-1- yl)pyrimido[5,4-d]pyrimidin-2- yl)azanediyl)diethanol | 616.779 |
| 39 | 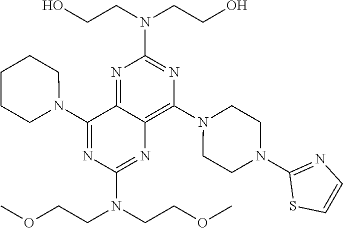 [SMILES: COCCN(CCOC)c1nc(N2CCCCC2)c2nc(N(CCO)CCO)nc(N3CCN(c4nccs4)CC3)c2n1] | 2,2′-((6-(bis(2-methoxyethyl)amino)-8- (piperidin-1-yl)-4-(4-(thiazol-2-yl)piperazin-1- yl)pyrimido[5,4-d]pyrimidin-2- yl)azanediyl)diethanol | 616.779 |
| 44 | 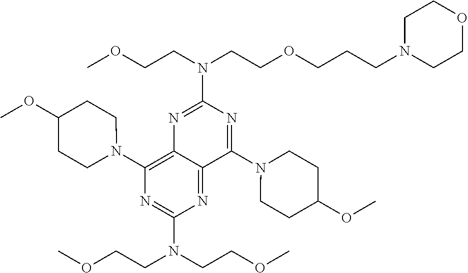 [SMILES: COCCN(CCOC)c1nc(N2CCC(OC)CC2)c2nc(N(CCOC)CCOCCCN3CCOCC3)nc(N3CCC(OC)CC3)c2n1] | N2,N2,N6-tris(2-methoxyethyl)-4,8-bis(4- methoxypiperidin-1-yl)-N6-(2-(3- morpholinopropoxy)ethyl)pyrimido[5,4- d]pyrimidine-2,6-diamine | 733.942 |
| 45 | 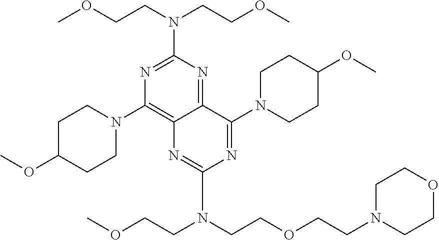 [SMILES: COCCN(CCOC)c1nc(N2CCC(OC)CC2)c2nc(N(CCOC)CCOCCN3CCOCC3)nc(N3CCC(OC)CC3)c2n1] | N2,N2,N6-tris(2-methoxyethyl)-4,8-bis(4- methoxypiperidin-1-yl)-N6-(2-(2- morpholinoethoxy)ethyl)pyrimido[5,4- d]pyrimidine-2,6-diamine | 719.915 |
| 46 | 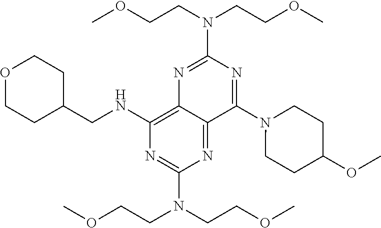 [SMILES: COCCN(CCOC)c1nc(N2CCC(OC)CC2)c2nc(N(CCOC)CCOC)nc(NCC3CCOCC3)c2n1] | N2,N2,N6,N6-tetrakis(2-methoxyethyl)-8-(4- methoxypiperidin-1-yl)-N4-((tetrahydro-2H- pyran-4-yl)methyl)pyrimido[5,4-d]pyrimidine- 2,4,6-triamine | 620.784 |
| 47 | 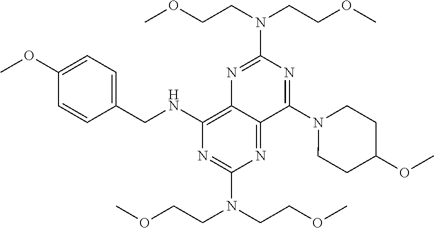 | N4-(4-methoxybenzyl)-N2,N2,N6,N6- tetrakis(2-methoxyethyl)-8-(4- methoxypiperidin-1-yl)pyrimido[5,4- d]pyrimidine-2,4,6-triamine | 642.789 |
| 48 | 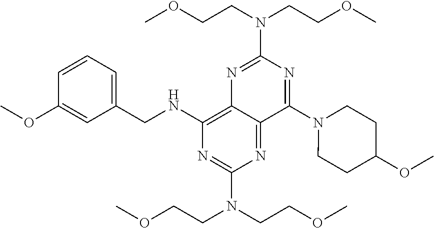 | N4-(3-methoxybenzyl)-N2,N2,N6,N6- tetrakis(2-methoxyethyl)-8-(4- methoxypiperidin-1-yl)pyrimido[5,4- d]pyrimidine-2,4,6-triamine | 642.789 |
| 49 | 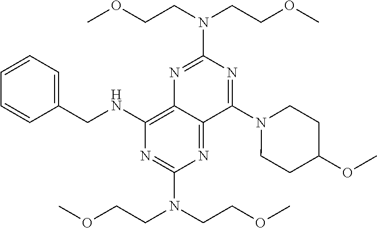 [SMILES: COCCN(CCOC)c1nc(N2CCC(OC)CC2)c2nc(N(CCOC)CCOC)nc(NCc3ccccc3)c2n1] | N4-benzyl-N2,N2,N6,N6-tetrakis(2- methoxyethyl)-8-(4-methoxypiperidin-1- yl)pyrimido[5,4-d]pyrimidine-2,4,6-triamine | 612.763 |
| 50 | 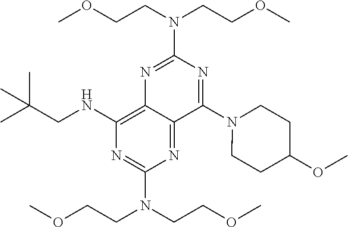 [SMILES: COCCN(CCOC)c1nc(N2CCC(OC)CC2)c2nc(N(CCOC)CCOC)nc(NCC(C)(C)C)c2n1] | N2,N2,N6,N6-tetrakis(2-methoxyethyl)-8-(4- methoxypiperidin-1-yl)-N4- neopentylpyrimido[5,4-d]pyrimidine-2,4,6- triamine | 592.774 |
| 51 | 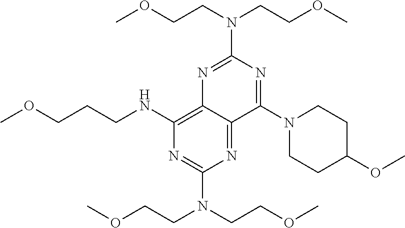 | N2,N2,N6,N6-tetrakis(2-methoxyethyl)-8-(4- methoxypiperidin-1-yl)-N4-(3- methoxypropyl)pyrimido[5,4-d]pyrimidine- 2,4,6-triamine | 594.747 |
| 52 | 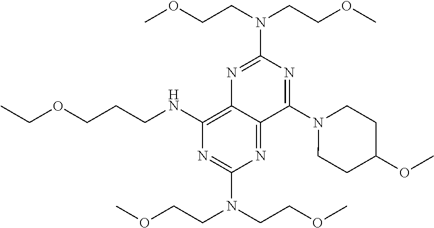 [SMILES: CCOCCCNc1nc(N(CCOC)CCOC)nc2c(N3CCC(OC)CC3)nc(N(CCOC)CCOC)nc12] | N4-(3-ethoxypropyl)-N2,N2,N6,N6-tetrakis(2- methoxyethyl)-8-(4-methoxypiperidin-1- yl)pyrimido[5,4-d]pyrimidine-2,4,6-triamine | 608.773 |
| 53 | 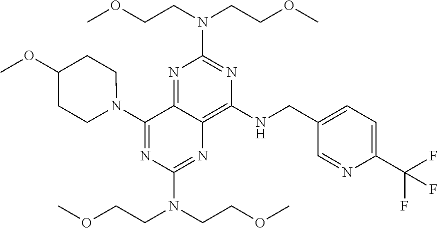 [SMILES: COCCN(CCOC)c1nc(N2CCC(OC)CC2)c2nc(N(CCOC)CCOC)nc(NCc3ccc(C(F)(F)F)nc3)c2n1] | N2,N2,N6,N6-tetrakis(2-methoxyethyl)-8-(4- methoxypiperidin-1-yl)-N4-((6- (trifluoromethyl)pyridin-3- yl)methyl)pyrimido[5,4-d]pyrimidine-2,4,6- triamine | 681.749 |
| 54 | 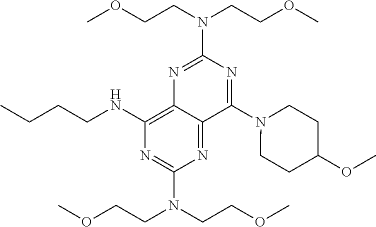 [SMILES: CCCCNc1nc(N(CCOC)CCOC)nc2c(N3CCC(OC)CC3)nc(N(CCOC)CCOC)nc12] | N4-butyl-N2,N2,N6,N6-tetrakis(2- methoxyethyl)-8-(4-methoxypiperidin-1- yl)pyrimido[5,4-d]pyrimidine-2,4,6-triamine | 578.747 |
| 55 | 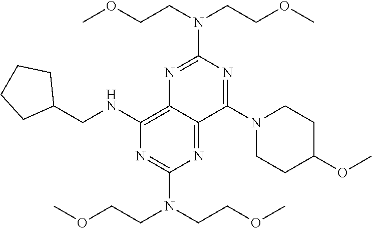 [SMILES: COCCN(CCOC)c1nc(N2CCC(OC)CC2)c2nc(N(CCOC)CCOC)nc(NCC3CCCC3)c2n1] | N4-(cyclopentylmethyl)-N2,N2,N6,N6- tetrakis(2-methoxyethyl)-8-(4- methoxypiperidin-1-yl)pyrimido[5,4- d]pyrimidine-2,4,6-triamine | 604.784 |
| 56 | 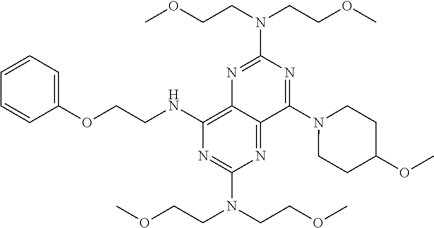 [SMILES: COCCN(CCOC)c1nc(N2CCC(OC)CC2)c2nc(N(CCOC)CCOC)nc(NCCOc3ccccc3)c2n1] | N2,N2,N6,N6-tetrakis(2-methoxyethyl)-8-(4- methoxypiperidin-1-yl)-N4-(2- phenoxyethyl)pyrimido[5,4-d]pyrimidine- 2,4,6-triamine | 642.789 |
| 57 | 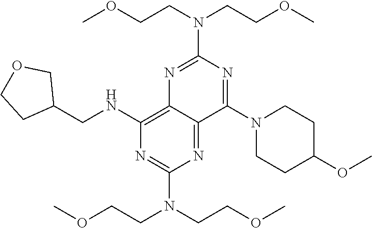 [SMILES: COCCN(CCOC)c1nc(N2CCC(OC)CC2)c2nc(N(CCOC)CCOC)nc(NCC3CCOC3)c2n1] | N2,N2,N6,N6-tetrakis(2-methoxyethyl)-8-(4- methoxypiperidin-1-yl)-N4-((tetrahydrofuran- 3-yl)methyl)pyrimido[5,4-d]pyrimidine-2,4,6- triamine | 606.757 |
| 58 | 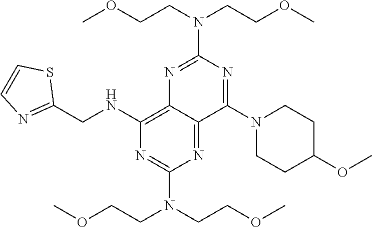 [SMILES: COCCN(CCOC)c1nc(N2CCC(OC)CC2)c2nc(N(CCOC)CCOC)nc(NCc3nccs3)c2n1] | N2,N2,N6,N6-tetrakis(2-methoxyethyl)-8-(4- methoxypiperidin-1-yl)-N4-(thiazol-2- ylmethyl)pyrimido[5,4-d]pyrimidine-2,4,6- triamine | 619.779 |
| 59 | 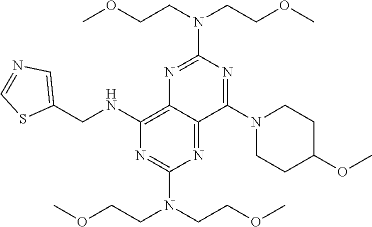 [SMILES: COCCN(CCOC)c1nc(N2CCC(OC)CC2)c2nc(N(CCOC)CCOC)nc(NCc3cncs3)c2n1] | N2,N2,N6,N6-tetrakis(2-methoxyethyl)-8-(4- methoxypiperidin-1-yl)-N4-(thiazol-5- ylmethyl)pyrimido[5,4-d]pyrimidine-2,4,6- triamine | 619.779 |
| 60 | 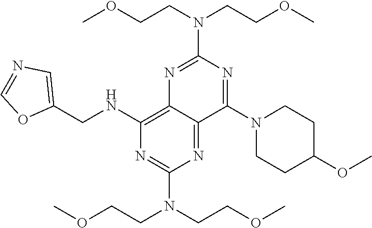 [SMILES: COCCN(CCOC)c1nc(N2CCC(OC)CC2)c2nc(N(CCOC)CCOC)nc(NCc3cnco3)c2n1] | N2,N2,N6,N6-tetrakis(2-methoxyethyl)-8-(4- methoxypiperidin-1-yl)-N4-(oxazol-5- ylmethyl)pyrimido[5,4-d]pyrimidine-2,4,6- triamine | 603.714 |
| 61 | 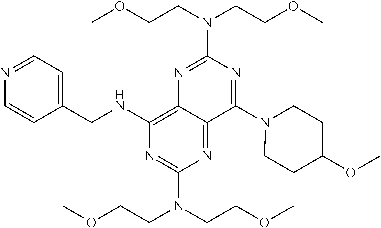 [SMILES: COCCN(CCOC)c1nc(N2CCC(OC)CC2)c2nc(N(CCOC)CCOC)nc(NCc3ccncc3)c2n1] | N2,N2,N6,N6-tetrakis(2-methoxyethyl)-8-(4- methoxypiperidin-1-yl)-N4-(pyridin-4- ylmethyl)pyrimido[5,4-d]pyrimidine-2,4,6- triamine | 613.751 |
| 62 | 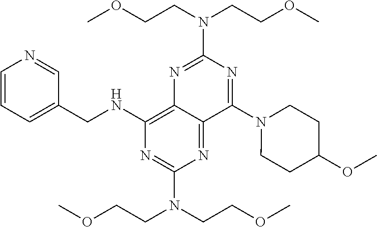 [SMILES: COCCN(CCOC)c1nc(N2CCC(OC)CC2)c2nc(N(CCOC)CCOC)nc(NCc3cccnc3)c2n1] | N2,N2,N6,N6-tetrakis(2-methoxyethyl)-8-(4- methoxypiperidin-1-yl)-N4-(pyridin-3- ylmethyl)pyrimido[5,4-d]pyrimidine-2,4,6- triamine | 613.751 |
| 63 | 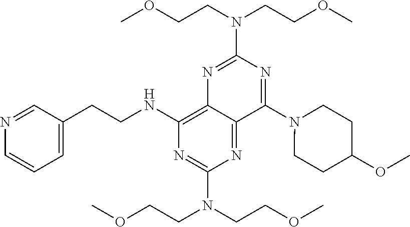 [SMILES: COCCN(CCOC)c1nc(N2CCC(OC)CC2)c2nc(N(CCOC)CCOC)nc(NCCc3cccnc3)c2n1] | N2,N2,N6,N6-tetrakis(2-methoxyethyl)-8-(4- methoxypiperidin-1-yl)-N4-(2-(pyridin-3- yl)ethyl)pyrimido[5,4-d]pyrimidine-2,4,6- triamine | 627.778 |
| 64 | 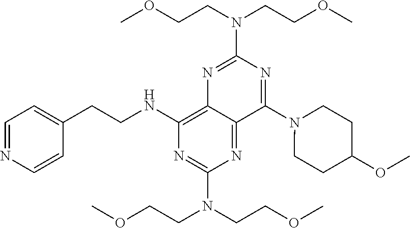 [SMILES: COCCN(CCOC)c1nc(N2CCC(OC)CC2)c2nc(N(CCOC)CCOC)nc(NCCc3ccncc3)c2n1] | N2,N2,N6,N6-tetrakis(2-methoxyethyl)-8-(4- methoxypiperidin-1-yl)-N4-(2-(pyridin-4- yl)ethyl)pyrimido[5,4-d]pyrimidine-2,4,6- triamine | 627.778 |
| 65 | 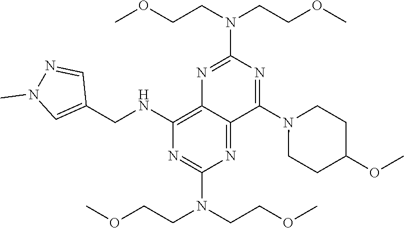 | N2,N2,N6,N6-tetrakis(2-methoxyethyl)-8-(4- methoxypiperidin-1-yl)-N4-((1-methyl-1H- pyrazol-4-yl)methyl)pyrimido[5,4- d]pyrimidine-2,4,6-triamine | 616.755 |
| 66 | 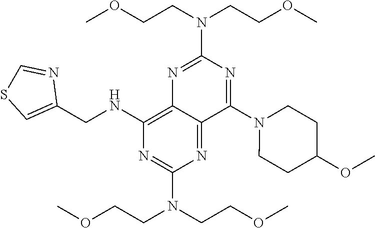 [SMILES: COCCN(CCOC)c1nc(N2CCC(OC)CC2)c2nc(N(CCOC)CCOC)nc(NCc3cscn3)c2n1] | N2,N2,N6,N6-tetrakis(2-methoxyethyl)-8-(4- methoxypiperidin-1-yl)-N4-(thiazol-4- ylmethyl)pyrimido[5,4-d]pyrimidine-2,4,6- triamine | 619.779 |
| 67 | 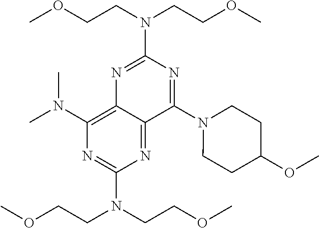 | N2,N2,N6,N6-tetrakis(2-methoxyethyl)-8-(4- methoxypiperidin-1-yl)-N4,N4- dimethylpyrimido[5,4-d]pyrimidine-2,4,6- triamine | 550.694 |
| 68 | 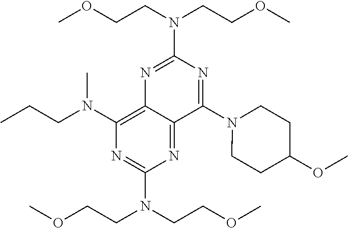 [SMILES: CCCN(C)c1nc(N(CCOC)CCOC)nc2c(N3CCC(OC)CC3)nc(N(CCOC)CCOC)nc12] | N2,N2,N6,N6-tetrakis(2-methoxyethyl)-8-(4- methoxypiperidin-1-yl)-N4-methyl-N4- propylpyrimido[5,4-d]pyrimidine-2,4,6- triamine | 578.747 |
| 69 | 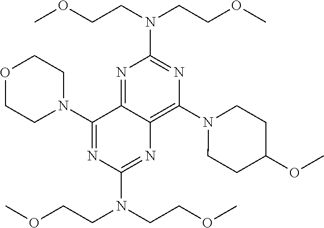 | N2,N2,N6,N6-tetrakis(2-methoxyethyl)-4-(4- methoxypiperidin-1-yl)-8- morpholinopyrimido[5,4-d]pyrimidine-2,6- diamine | 592.731 |
| 70 | 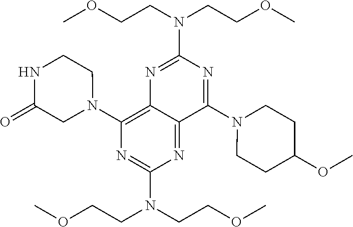 [SMILES: COCCN(CCOC)c1nc(N2CCNC(=O)C2)c2nc(N(CCOC)CCOC)nc(N3CCC(OC)CC3)c2n1] | 4-(2,6-bis(bis(2-methoxyethyl)amino)-8-(4- methoxypiperidin-1-yl)pyrimido[5,4- d]pyrimidin-4-yl)piperazin-2-one | 605.729 |
| 71 | 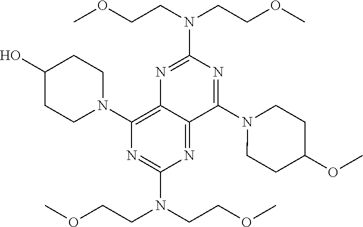 [SMILES: COCCN(CCOC)c1nc(N2CCC(OC)CC2)c2nc(N(CCOC)CCOC)nc(N3CCC(O)CC3)c2n1] | 1-(2,6-bis(bis(2-methoxyethyl)amino)-8-(4- methoxypiperidin-1-yl)pyrimido[5,4- d]pyrimidin-4-yl)piperidin-4-ol | 606.757 |
| 72 | 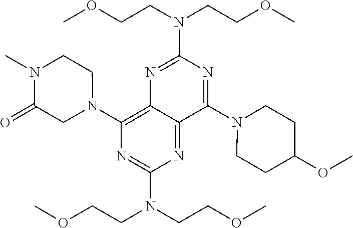 [SMILES: COCCN(CCOC)c1nc(N2CCN(C)C(=O)C2)c2nc(N(CCOC)CCOC)nc(N3CCC(OC)CC3)c2n1] | 4-(2,6-bis(bis(2-methoxyethyl)amino)-8-(4- methoxypiperidin-1-yl)pyrimido[5,4- d]pyrimidin-4-yl)-1-methylpiperazin-2-one | 619.756 |
| 73 | 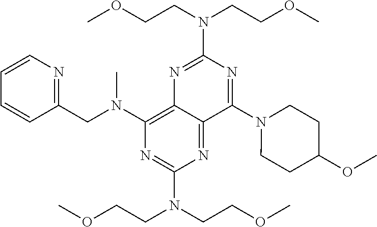 [SMILES: COCCN(CCOC)c1nc(N2CCC(OC)CC2)c2nc(N(CCOC)CCOC)nc(N(C)Cc3ccccn3)c2n1] | N2,N2,N6,N6-tetrakis(2-methoxyethyl)-8-(4- methoxypiperidin-1-yl)-N4-methyl-N4- (pyridin-2-ylmethyl)pyrimido[5,4- d]pyrimidine-2,4,6-triamine | 627.778 |
| 74 | 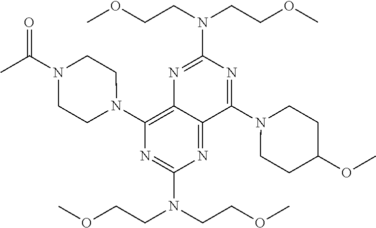 [SMILES: COCCN(CCOC)c1nc(N2CCN(C(C)=O)CC2)c2nc(N(CCOC)CCOC)nc(N3CCC(OC)CC3)c2n1] | 1-(4-(2,6-bis(bis(2-methoxyethyl)amino)-8-(4- methoxypiperidin-1-yl)pyrimido[5,4- d]pyrimidin-4-yl)piperazin-1-yl)ethanone | 633.783 |
| 75 | 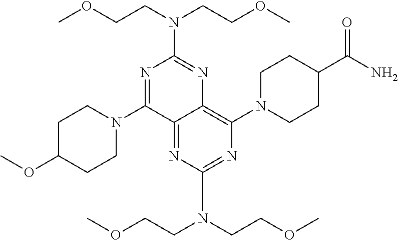 [SMILES: COCCN(CCOC)c1nc(N2CCC(C(N)=O)CC2)c2nc(N(CCOC)CCOC)nc(N3CCC(OC)CC3)c2n1] | 1-(2,6-bis(bis(2-methoxyethyl)amino)-8-(4- methoxypiperidin-1-yl)pyrimido[5,4- d]pyrimidin-4-yl)piperidine-4-carboxamide | 633.783 |
| 76 | 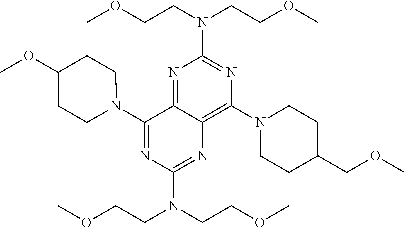 | N2,N2,N6,N6-tetrakis(2-methoxyethyl)-4-(4- (methoxymethyl)piperidin-1-yl)-8-(4- methoxypiperidin-1-yl)pyrimido[5,4- d]pyrimidine-2,6-diamine | 634.81 |
| 77 | 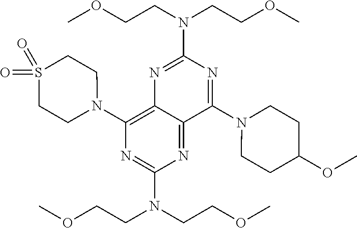 [SMILES: COCCN(CCOC)c1nc(N2CCS(=O)(=O)CC2)c2nc(N(CCOC)CCOC)nc(N3CCC(OC)CC3)c2n1] | 4-(2,6-bis(bis(2-methoxyethyl)amino)-8-(4- methoxypiperidin-1-yl)pyrimido[5,4- d]pyrimidin-4-yl)thiomorpholine 1,1-dioxide | 640.795 |
| 78 | 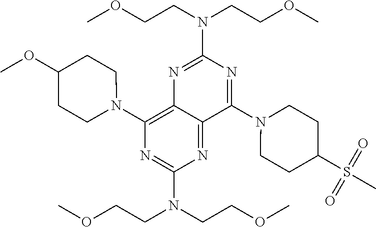 | N2,N2,N6,N6-tetrakis(2-methoxyethyl)-4-(4- methoxypiperidin-1-yl)-8-(4- (methylsulfonyl)piperidin-1-yl)pyrimido[5,4- d]pyrimidine-2,6-diamine | 668.848 |
| 79 | 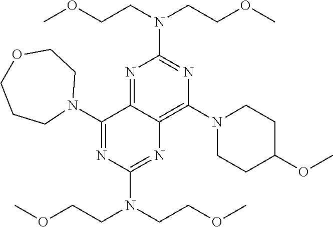 [SMILES: COCCN(CCOC)c1nc(N2CCC(OC)CC2)c2nc(N(CCOC)CCOC)nc(N3CCCOCC3)c2n1] | N2,N2,N6,N6-tetrakis(2-methoxyethyl)-4-(4- methoxypiperidin-1-yl)-8-(1,4-oxazepan-4- yl)pyrimido[5,4-d]pyrimidine-2,6-diamine | 606.757 |
| 80 | 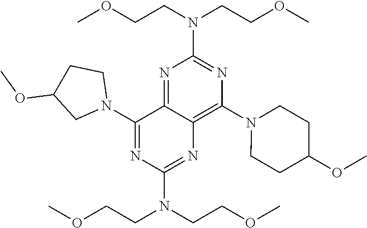 | N2,N2,N6,N6-tetrakis(2-methoxyethyl)-4-(4- methoxypiperidin-1-yl)-8-(3- methoxypyrrolidin-1-yl)pyrimido[5,4- d]pyrimidine-2,6-diamine | 606.757 |
| 81 | 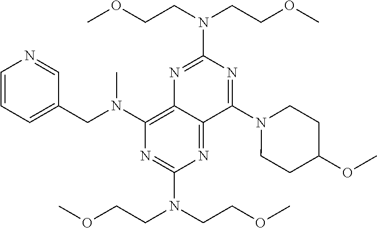 [SMILES: COCCN(CCOC)c1nc(N2CCC(OC)CC2)c2nc(N(CCOC)CCOC)nc(N(C)Cc3cccnc3)c2n1] | N2,N2,N6,N6-tetrakis(2-methoxyethyl)-8-(4- methoxypiperidin-1-yl)-N4-methyl-N4- (pyridin-3-ylmethyl)pyrimido[5,4- d]pyrimidine-2,4,6-triamine | 627.778 |
| 82 | 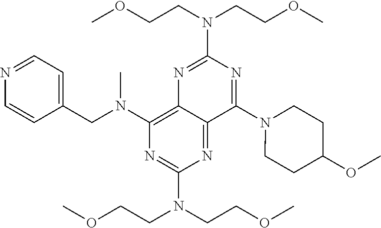 [SMILES: COCCN(CCOC)c1nc(N2CCC(OC)CC2)c2nc(N(CCOC)CCOC)nc(N(C)Cc3ccncc3)c2n1] | N2,N2,N6,N6-tetrakis(2-methoxyethyl)-8-(4- methoxypiperidin-1-yl)-N4-methyl-N4- (pyridin-4-ylmethyl)pyrimido[5,4- d]pyrimidine-2,4,6-triamine | 627.778 |
| 83 | 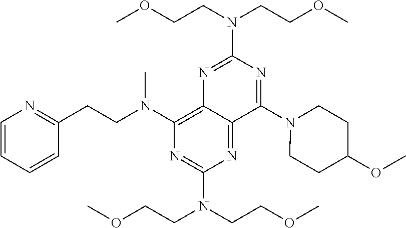 [SMILES: COCCN(CCOC)c1nc(N2CCC(OC)CC2)c2nc(N(CCOC)CCOC)nc(N(C)CCc3ccccn3)c2n1] | N2,N2,N6,N6-tetrakis(2-methoxyethyl)-8-(4- methoxypiperidin-1-yl)-N4-methyl-N4-(2- (pyridin-2-yl)ethyl)pyrimido[5,4-d]pyrimidine- 2,4,6-triamine | 641.805 |
| 84 | 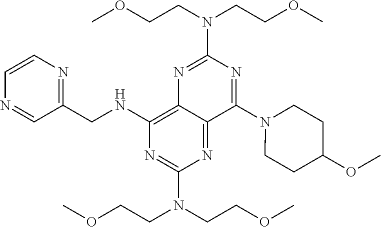 [SMILES: COCCN(CCOC)c1nc(N2CCC(OC)CC2)c2nc(N(CCOC)CCOC)nc(NCc3cnccn3)c2n1] | N2,N2,N6,N6-tetrakis(2-methoxyethyl)-8-(4- methoxypiperidin-1-yl)-N4-(pyrazin-2- ylmethyl)pyrimido[5,4-d]pyrimidine-2,4,6- triamine | 614.74 |
| 85 | 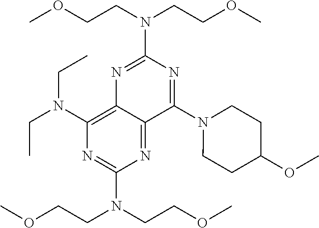 | N4,N4-diethyl-N2,N2,N6,N6-tetrakis(2- methoxyethyl)-8-(4-methoxypiperidin-1- yl)pyrimido[5,4-d]pyrimidine-2,4,6-triamine | 578.747 |
| 86 | 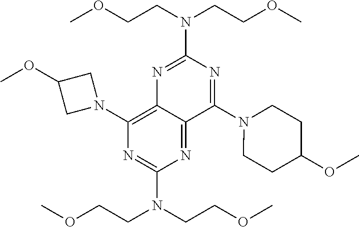 | 4-(3-methoxyazetidin-1-yl)-N2,N2,N6,N6- tetrakis(2-methoxyethyl)-8-(4- methoxypiperidin-1-yl)pyrimido[5,4- d]pyrimidine-2,6-diamine | 592.731 |
| 87 | 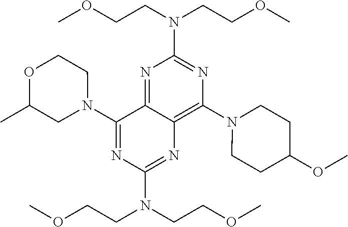 | N2,N2,N6,N6-tetrakis(2-methoxyethyl)-4-(4- methoxypiperidin-1-yl)-8-(2- methylmorpholino)pyrimido[5,4-d]pyrimidine- 2,6-diamine | 606.757 |
| 88 | 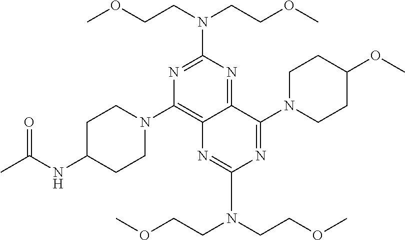 [SMILES: COCCN(CCOC)c1nc(N2CCC(OC)CC2)c2nc(N(CCOC)CCOC)nc(N3CCC(NC(C)=O)CC3)c2n1] | N-(1-(2,6-bis(bis(2-methoxyethyl)amino)-8-(4- methoxypiperidin-1-yl)pyrimido[5,4- d]pyrimidin-4-yl)piperidin-4-yl)acetamide | 647.809 |
| 89 | 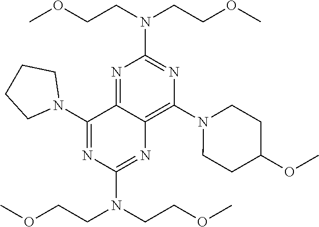 | N2,N2,N6,N6-tetrakis(2-methoxyethyl)-4-(4- methoxypiperidin-1-yl)-8-(pyrrolidin-1- yl)pyrimido[5,4-d]pyrimidine-2,6-diamine | 576.731 |
| 90 | 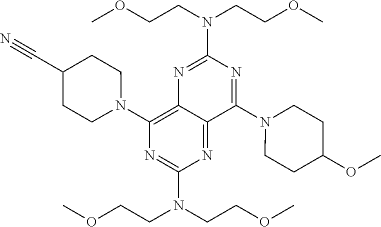 [SMILES: COCCN(CCOC)c1nc(N2CCC(OC)CC2)c2nc(N(CCOC)CCOC)nc(N3CCC(C#N)CC3)c2n1] | 1-(2,6-bis(bis(2-methoxyethyl)amino)-8-(4- methoxypiperidin-1-yl)pyrimido[5,4- d]pyrimidin-4-yl)piperidine-4-carbonitrile | 615.767 |
| 91 | 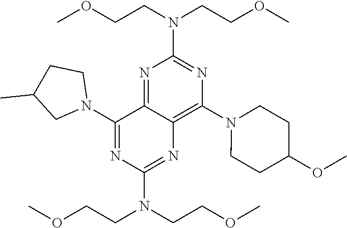 | N2,N2,N6,N6-tetrakis(2-methoxyethyl)-4-(4- methoxypiperidin-1-yl)-8-(3-methylpyrrolidin- 1-yl)pyrimido[5,4-d]pyrimidine-2,6-diamine | 590.758 |
| 92 | 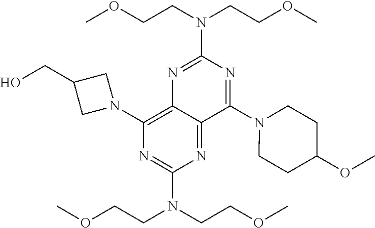 [SMILES: COCCN(CCOC)c1nc(N2CC(CO)C2)c2nc(N(CCOC)CCOC)nc(N3CCC(OC)CC3)c2n1] | (1-(2,6-bis(bis(2-methoxyethyl)amino)-8-(4- methoxypiperidin-1-yl)pyrimido[5,4- d]pyrimidin-4-yl)azetidin-3-yl)methanol | 592.731 |
| 93 | 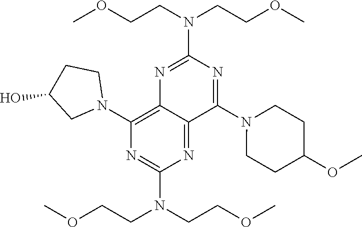 [SMILES: COCCN(CCOC)c1nc(N2CC[C@@H](O)C2)c2nc(N(CCOC)CCOC)nc(N3CCC(OC)CC3)c2n1] | (R)-1-(2,6-bis(bis(2-methoxyethyl)amino)-8- (4-methoxypiperidin-1-yl)pyrimido[5,4- d]pyrimidin-4-yl)pyrrolidin-3-ol | 592.731 |
| 94 | 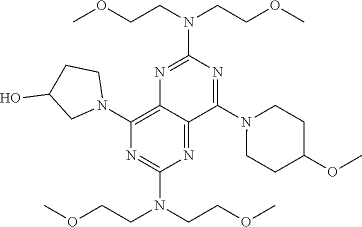 [SMILES: COCCN(CCOC)c1nc(N2CCC(O)C2)c2nc(N(CCOC)CCOC)nc(N3CCC(OC)CC3)c2n1] | 1-(2,6-bis(bis(2-methoxyethyl)amino)-8-(4- methoxypiperidin-1-yl)pyrimido[5,4- d]pyrimidin-4-yl)pyrrolidin-3-ol | 592.731 |
| 95 | 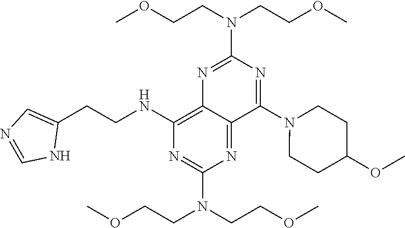 [SMILES: COCCN(CCOC)c1nc(N2CCC(OC)CC2)c2nc(N(CCOC)CCOC)nc(NCCc3cnc[nH]3)c2n1] | N4-(2-(1H-imidazol-5-yl)ethyl)-N2,N2,N6,N6- tetrakis(2-methoxyethyl)-8-(4- methoxypiperidin-1-yl)pyrimido[5,4- d]pyrimidine-2,4,6-triamine | 616.755 |
| 96 | 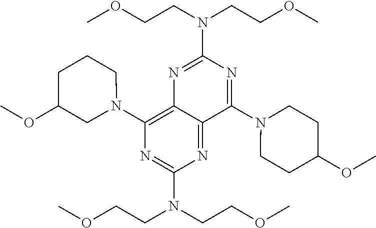 | N2,N2,N6,N6-tetrakis(2-methoxyethyl)-4-(3- methoxypiperidin-1-yl)-8-(4- methoxypiperidin-1-yl)pyrimido[5,4- d]pyrimidine-2,6-diamine | 620.784 |
| 97 | 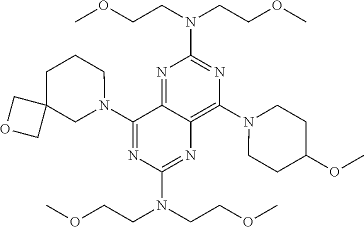 [SMILES: COCCN(CCOC)c1nc(N2CCCC3(COC3)C2)c2nc(N(CCOC)CCOC)nc(N3CCC(OC)CC3)c2n1] | N2,N2,N6,N6-tetrakis(2-methoxyethyl)-4-(4- methoxypiperidin-1-yl)-8-(2-oxa-6- azaspiro[3.5]nonan-6-yl)pyrimido[5,4- d]pyrimidine-2,6-diamine | 632.795 |
| 98 | 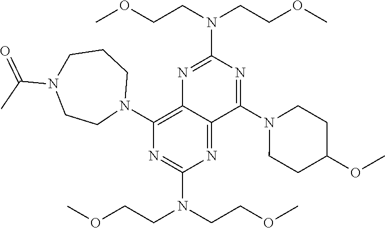 [SMILES: COCCN(CCOC)c1nc(N2CCCN(C(C)=O)CC2)c2nc(N(CCOC)CCOC)nc(N3CCC(OC)CC3)c2n1] | 1-(4-(2,6-bis(bis(2-methoxyethyl)amino)-8-(4- methoxypiperidin-1-yl)pyrimido[5,4- d]pyrimidin-4-yl)-1,4-diazepan-1-yl)ethanone | 647.809 |
| 99 | 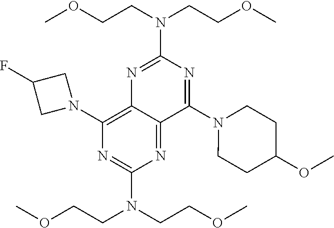 [SMILES: COCCN(CCOC)c1nc(N2CC(F)C2)c2nc(N(CCOC)CCOC)nc(N3CCC(OC)CC3)c2n1] | 4-(3-fluoroazetidin-1-yl)-N2,N2,N6,N6- tetrakis(2-methoxyethyl)-8-(4- methoxypiperidin-1-yl)pyrimido[5,4- d]pyrimidine-2,6-diamine | 580.695 |
| 100 | 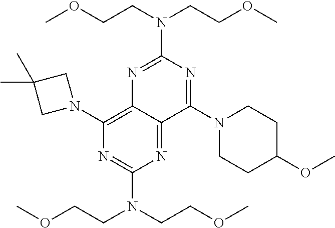 | 4-(3,3-dimethylazetidin-1-yl)-N2,N2,N6,N6- tetrakis(2-methoxyethyl)-8-(4- methoxypiperidin-1-yl)pyrimido[5,4- d]pyrimidine-2,6-diamine | 590.758 |
| 101 | 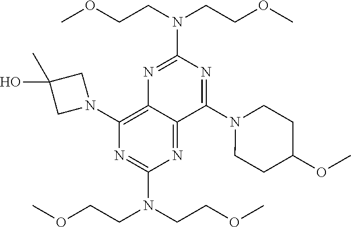 [SMILES: COCCN(CCOC)c1nc(N2CC(C)(O)C2)c2nc(N(CCOC)CCOC)nc(N3CCC(OC)CC3)c2n1] | 1-(2,6-bis(bis(2-methoxyethyl)amino)-8-(4- methoxypiperidin-1-yl)pyrimido[5,4- d]pyrimidin-4-yl)-3-methylazetidin-3-ol | 592.731 |
| 102 | 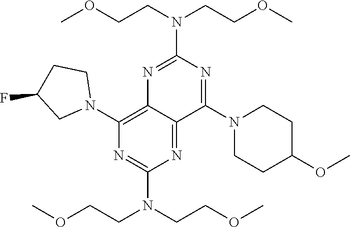 [SMILES: COCCN(CCOC)c1nc(N2CC[C@H](F)C2)c2nc(N(CCOC)CCOC)nc(N3CCC(OC)CC3)c2n1] | (S)-4-(3-fluoropyrrolidin-1-yl)-N2,N2,N6,N6- tetrakis(2-methoxyethyl)-8-(4- methoxypiperidin-1-yl)pyrimido[5,4- d]pyrimidine-2,6-diamine | 594.722 |
| 103 | 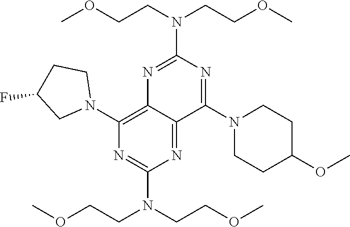 [SMILES: COCCN(CCOC)c1nc(N2CC[C@@H](F)C2)c2nc(N(CCOC)CCOC)nc(N3CCC(OC)CC3)c2n1] | (R)-4-(3-fluoropyrrolidin-1-yl)-N2,N2,N6,N6- tetrakis(2-methoxyethyl)-8-(4- methoxypiperidin-1-yl)pyrimido[5,4- d]pyrimidine-2,6-diamine | 594.722 |
| 104 | 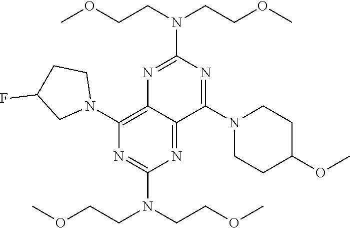 [SMILES: COCCN(CCOC)c1nc(N2CCC(F)C2)c2nc(N(CCOC)CCOC)nc(N3CCC(OC)CC3)c2n1] | 4-(3-fluoropyrrolidin-1-yl)-N2,N2,N6,N6- tetrakis(2-methoxyethyl)-8-(4- methoxypiperidin-1-yl)pyrimido[5,4- d]pyrimidine-2,6-diamine | 594.722 |
| 105 | 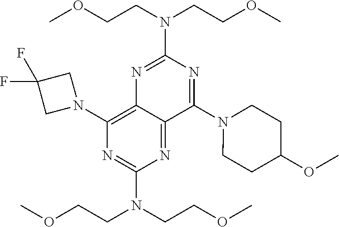 [SMILES: COCCN(CCOC)c1nc(N2CC(F)(F)C2)c2nc(N(CCOC)CCOC)nc(N3CCC(OC)CC3)c2n1] | 4-(3,3-difluoroazetidin-1-yl)-N2,N2,N6,N6- tetrakis(2-methoxyethyl)-8-(4- methoxypiperidin-1-yl)pyrimido[5,4- d]pyrimidine-2,6-diamine | 598.686 |
| 106 | 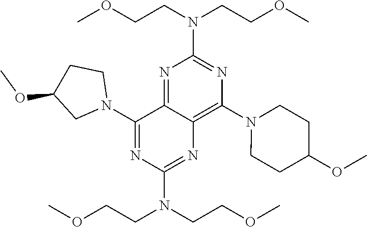 | (S)-N2,N2,N6,N6-tetrakis(2-methoxyethyl)-4- (4-methoxypiperidin-1-yl)-8-(3- methoxypyrrolidin-1-yl)pyrimido[5,4- d]pyrimidine-2,6-diamine | 606.757 |
| 107 | 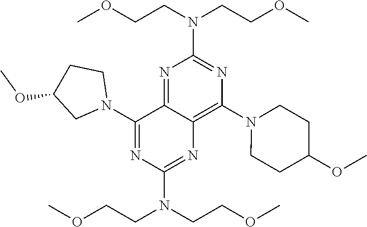 | (R)-N2,N2,N6,N6-tetrakis(2-methoxyethyl)-4- (4-methoxypiperidin-1-yl)-8-(3- methoxypyrrolidin-1-yl)pyrimido[5,4- d]pyrimidine-2,6-diamine | 606.757 |
| 108 | 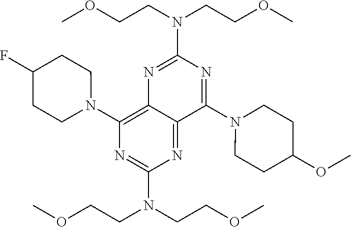 [SMILES: COCCN(CCOC)c1nc(N2CCC(OC)CC2)c2nc(N(CCOC)CCOC)nc(N3CCC(F)CC3)c2n1] | 4-(4-fluoropiperidin-1-yl)-N2,N2,N6,N6- tetrakis(2-methoxyethyl)-8-(4- methoxypiperidin-1-yl)pyrimido[5,4- d]pyrimidine-2,6-diamine | 608.748 |
| 109 | 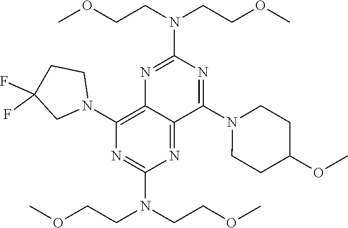 [SMILES: COCCN(CCOC)c1nc(N2CCC(F)(F)C2)c2nc(N(CCOC)CCOC)nc(N3CCC(OC)CC3)c2n1] | 4-(3,3-difluoropyrrolidin-1-yl)-N2,N2,N6,N6- tetrakis(2-methoxyethyl)-8-(4- methoxypiperidin-1-yl)pyrimido[5,4- d]pyrimidine-2,6-diamine | 612.712 |
| 110 | 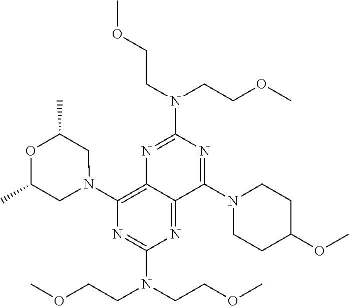 | 4-((2R,6S)-2,6-dimethylmorpholino)- N2,N2,N6,N6-tetrakis(2-methoxyethyl)-8-(4- methoxypiperidin-1-yl)pyrimido[5,4- d]pyrimidine-2,6-diamine | 620.784 |
| 111 | 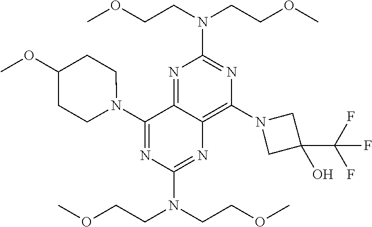 [SMILES: COCCN(CCOC)c1nc(N2CC(O)(C(F)(F)F)C2)c2nc(N(CCOC)CCOC)nc(N3CCC(OC)CC3)c2n1] | 1-(2,6-bis(bis(2-methoxyethyl)amino)-8-(4- methoxypiperidin-1-yl)pyrimido[5,4- d]pyrimidin-4-yl)-3-(trifluoromethyl)azetidin- 3-ol | 646.702 |
| 112 | 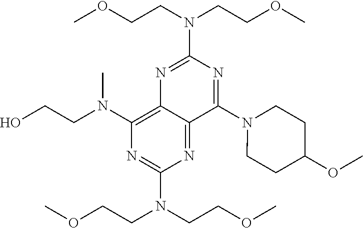 [SMILES: COCCN(CCOC)c1nc(N2CCC(OC)CC2)c2nc(N(CCOC)CCOC)nc(N(C)CCO)c2n1] | 2-((2,6-bis(bis(2-methoxyethyl)amino)-8-(4- methoxypiperidin-1-yl)pyrimido[5,4- d]pyrimidin-4-yl)(methyl)amino)ethanol | 580.72 |
| 113 | 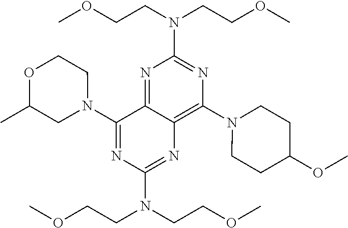 | N2,N2,N6,N6-tetrakis(2-methoxyethyl)-4-(4- methoxypiperidin-1-yl)-8-(2- methylmorpholino)pyrimido[5,4-d]pyrimidine- 2,6-diamine | 606.757 |
| 114 | 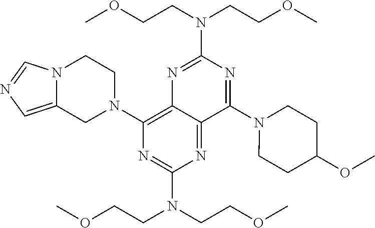 [SMILES: COCCN(CCOC)c1nc(N2CCn3cncc3C2)c2nc(N(CCOC)CCOC)nc(N3CCC(OC)CC3)c2n1] | 4-(5,6-dihydroimidazo[1,5-a]pyrazin-7(8H)- yl)-N2,N2,N6,N6-tetrakis(2-methoxyethyl)-8- (4-methoxypiperidin-1-yl)pyrimido[5,4- d]pyrimidine-2,6-diamine | 628.766 |
| 115 | 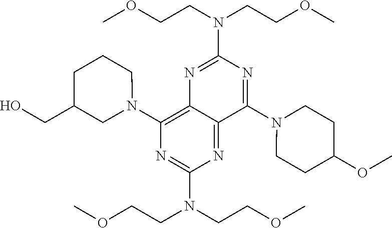 [SMILES: COCCN(CCOC)c1nc(N2CCCC(CO)C2)c2nc(N(CCOC)CCOC)nc(N3CCC(OC)CC3)c2n1] | (1-(2,6-bis(bis(2-methoxyethyl)amino)-8-(4- methoxypiperidin-1-yl)pyrimido[5,4- d]pyrimidin-4-yl)piperidin-3-yl)methanol | 620.784 |
| 116 | 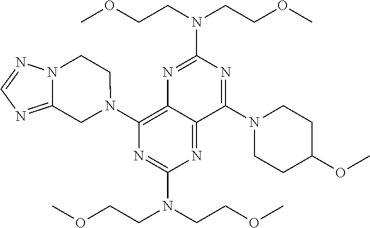 [SMILES: COCCN(CCOC)c1nc(N2CCn3ncnc3C2)c2nc(N(CCOC)CCOC)nc(N3CCC(OC)CC3)c2n1] | 4-(5,6-dihydro-[1,2,4]triazolo[1,5-a]pyrazin- 7(8H)-yl)-N2,N2,N6,N6-tetrakis(2- methoxyethyl)-8-(4-methoxypiperidin-1- yl)pyrimido[5,4-d]pyrimidine-2,6-diamine | 629.754 |
| 117 | 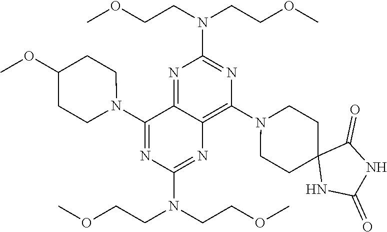 [SMILES: COCCN(CCOC)c1nc(N2CCC3(CC2)NC(=O)NC3=O)c2nc(N(CCOC)CCOC)nc(N3CCC(OC)CC3)c2n1] | 8-(2,6-bis(bis(2-methoxyethyl)amino)-8-(4- methoxypiperidin-1-yl)pyrimido[5,4- d]pyrimidin-4-yl)-1,3,8-triazaspiro[4.5]decane- 2,4-dione | 674.792 |
| 118 | 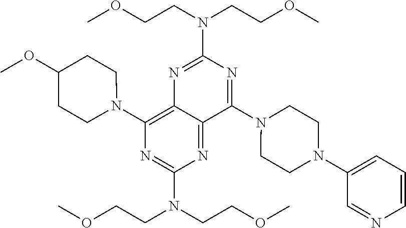 [SMILES: COCCN(CCOC)c1nc(N2CCN(c3cccnc3)CC2)c2nc(N(CCOC)CCOC)nc(N3CCC(OC)CC3)c2n1] | N2,N2,N6,N6-tetrakis(2-methoxyethyl)-4-(4- methoxypiperidin-1-yl)-8-(4-(pyridin-3- yl)piperazin-1-yl)pyrimido[5,4-d]pyrimidine- 2,6-diamine | 668.83 |
| 119 | 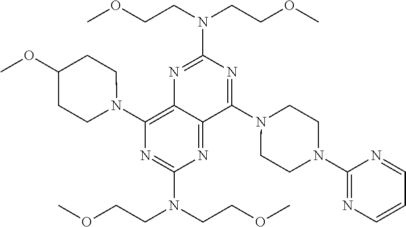 [SMILES: COCCN(CCOC)c1nc(N2CCN(c3ncccn3)CC2)c2nc(N(CCOC)CCOC)nc(N3CCC(OC)CC3)c2n1] | N2,N2,N6,N6-tetrakis(2-methoxyethyl)-4-(4- methoxypiperidin-1-yl)-8-(4-(pyrimidin-2- yl)piperazin-1-yl)pyrimido[5,4-d]pyrimidine- 2,6-diamine | 669.818 |
| 120 | 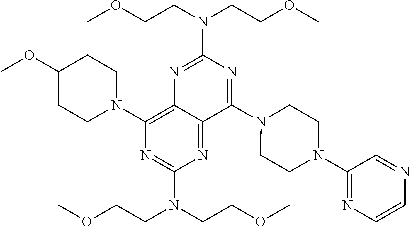 [SMILES: COCCN(CCOC)c1nc(N2CCN(c3cnccn3)CC2)c2nc(N(CCOC)CCOC)nc(N3CCC(OC)CC3)c2n1] | N2,N2,N6,N6-tetrakis(2-methoxyethyl)-4-(4- methoxypiperidin-1-yl)-8-(4-(pyrazin-2- yl)piperazin-1-yl)pyrimido[5,4-d]pyrimidine- 2,6-diamine | 669.818 |
| 121 | 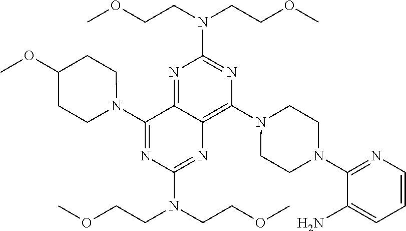 [SMILES: COCCN(CCOC)c1nc(N2CCN(c3ncccc3N)CC2)c2nc(N(CCOC)CCOC)nc(N3CCC(OC)CC3)c2n1] | 4-(4-(3-aminopyridin-2-yl)piperazin-1-yl)- N2,N2,N6,N6-tetrakis(2-methoxyethyl)-8-(4- methoxypiperidin-1-yl)pyrimido[5,4- d]pyrimidine-2,6-diamine | 683.845 |
| 122 | 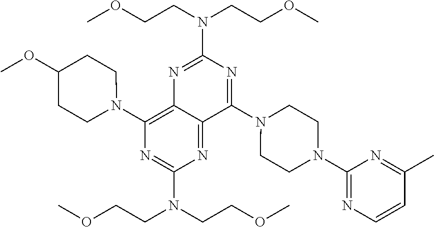 | N2,N2,N6,N6-tetrakis(2-methoxyethyl)-4-(4- methoxypiperidin-1-yl)-8-(4-(4- methylpyrimidin-2-yl)piperazin-1- yl)pyrimido[5,4-d]pyrimidine-2,6-diamine | 683.845 |
| 123 | 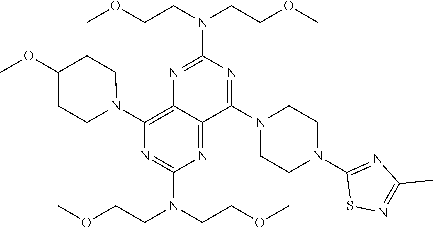 | N2,N2,N6,N6-tetrakis(2-methoxyethyl)-4-(4- methoxypiperidin-1-yl)-8-(4-(3-methyl-1,2,4- thiadiazol-5-yl)piperazin-1-yl)pyrimido[5,4- d]pyrimidine-2,6-diamine | 689.872 |
| 124 | 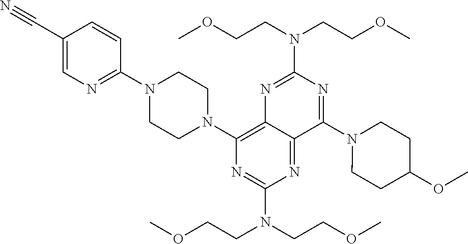 [SMILES: COCCN(CCOC)c1nc(N2CCN(c3ccc(C#N)cn3)CC2)c2nc(N(CCOC)CCOC)nc(N3CCC(OC)CC3)c2n1] | 6-(4-(2,6-bis(bis(2-methoxyethyl)amino)-8-(4- methoxypiperidin-1-yl)pyrimido[5,4- d]pyrimidin-4-yl)piperazin-1-yl)nicotinonitrile | 693.839 |
| 125 | 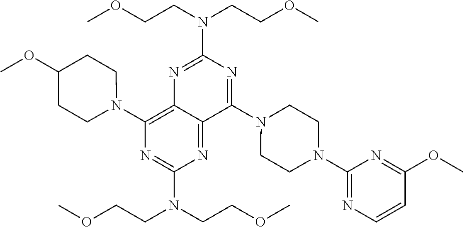 | N2,N2,N6,N6-tetrakis(2-methoxyethyl)-4-(4- methoxypiperidin-1-yl)-8-(4-(4- methoxypyrimidin-2-yl)piperazin-1- yl)pyrimido[5,4-d]pyrimidine-2,6-diamine | 699.844 |
| 126 | 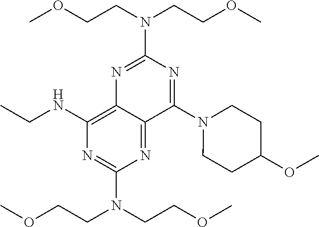 | N4-ethyl-N2,N2,N6,N6-tetrakis(2- methoxyethyl)-8-(4-methoxypiperidin-1- yl)pyrimido[5,4-d]pyrimidine-2,4,6-triamine | 550.694 |
| 127 | 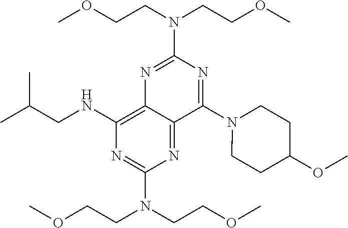 [SMILES: COCCN(CCOC)c1nc(N2CCC(OC)CC2)c2nc(N(CCOC)CCOC)nc(NCC(C)C)c2n1] | N4-isobutyl-N2,N2,N6,N6-tetrakis(2- methoxyethyl)-8-(4-methoxypiperidin-1- yl)pyrimido[5,4-d]pyrimidine-2,4,6-triamine | 578.747 |
| 128 | 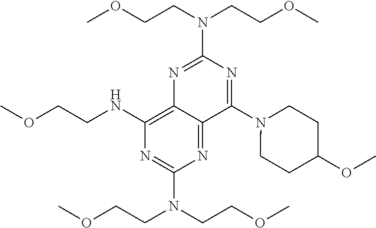 | N2,N2,N4,N6,N6-pentakis(2-methoxyethyl)-8- (4-methoxypiperidin-1-yl)pyrimido[5,4- d]pyrimidine-2,4,6-triamine | 580.72 |
| 129 | 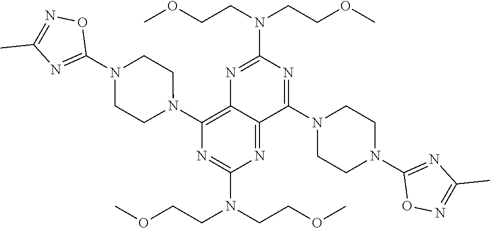 | N2,N2,N6,N6-tetrakis(2-methoxyethyl)-4,8- bis(4-(3-methyl-1,2,4-oxadiazol-5- yl)piperazin-1-yl)pyrimido[5,4-d]pyrimidine- 2,6-diamine | 726.83 |
| 130 | 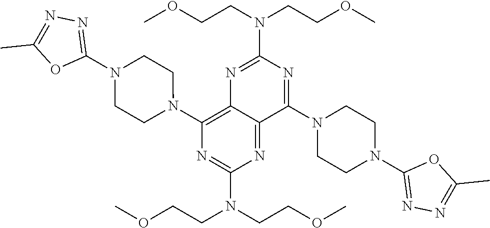 | N2,N2,N6,N6-tetrakis(2-methoxyethyl)-4,8- bis(4-(5-methyl-1,3,4-oxadiazol-2- yl)piperazin-1-yl)pyrimido[5,4-d]pyrimidine- 2,6-diamine | 726.83 |
| 131 | 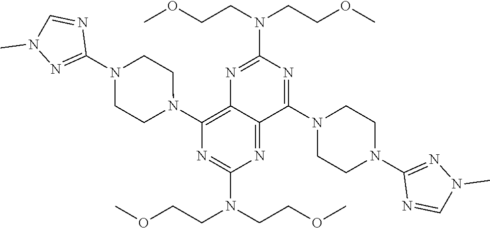 | N2,N2,N6,N6-tetrakis(2-methoxyethyl)-4,8- bis(4-(1-methyl-1H-1,2,4-triazol-3- yl)piperazin-1-yl)pyrimido[5,4-d]pyrimidine- 2,6-diamine | 724.86 |
| 132 | 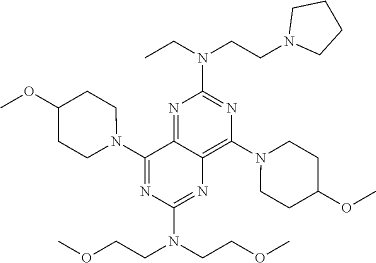 | N2-ethyl-N6,N6-bis(2-methoxyethyl)-4,8- bis(4-methoxypiperidin-1-yl)-N2-(2- (pyrrolidin-1-yl)ethyl)pyrimido[5,4- d]pyrimidine-2,6-diamine | 629.837 |
| 133 | 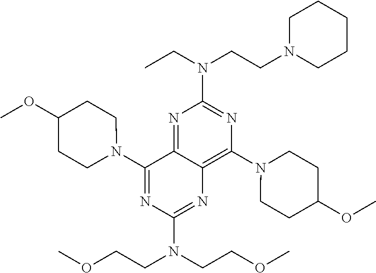 | N2-ethyl-N6,N6-bis(2-methoxyethyl)-4,8- bis(4-methoxypiperidin-1-yl)-N2-(2- (piperidin-1-yl)ethyl)pyrimido[5,4- d]pyrimidine-2,6-diamine | 643.864 |
| 134 | 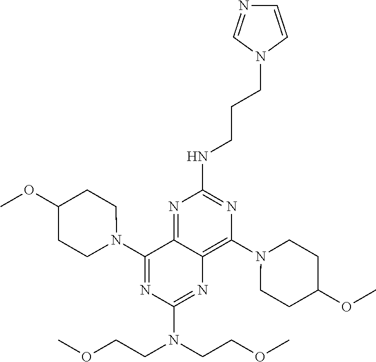 | N2-(3-(1H-imidazol-1-yl)propyl)-N6,N6-bis(2- methoxyethyl)-4,8-bis(4-methoxypiperidin-1- yl)pyrimido[5,4-d]pyrimidine-2,6-diamine | 612.767 |
| 135 | 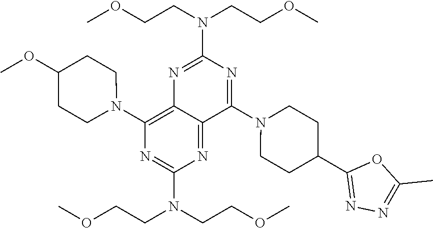 | N2,N2,N6,N6-tetrakis(2-methoxyethyl)-4-(4- methoxypiperidin-1-yl)-8-(4-(5-methyl-1,3,4- oxadiazol-2-yl)piperazin-1-yl)pyrimido[5,4- d]pyrimidine-2,6-diamine | 673.807 |
| 136 | 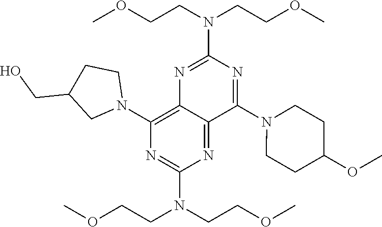 [SMILES: COCCN(CCOC)c1nc(N2CCC(CO)C2)c2nc(N(CCOC)CCOC)nc(N3CCC(OC)CC3)c2n1] | (1-(2,6-bis(bis(2-methoxyethyl)amino)-8-(4- methoxypiperidin-1-yl)pyrimido[5,4- d]pyrimidin-4-yl)pyrrolidin-3-yl)methanol | 606.757 |
| 137 | 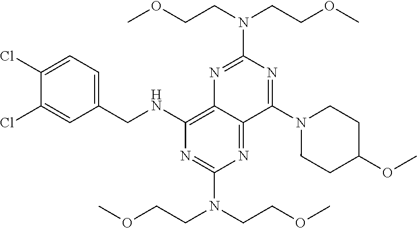 [SMILES: COCCN(CCOC)c1nc(N2CCC(OC)CC2)c2nc(N(CCOC)CCOC)nc(NCc3ccc(Cl)c(Cl)c3)c2n1] | N4-(3,4-dichlorobenzyl)-N2,N2,N6,N6- tetrakis(2-methoxyethyl)-8-(4- methoxypiperidin-1-yl)pyrimido[5,4- d]pyrimidine-2,4,6-triamine | 681.654 |
| 138 | 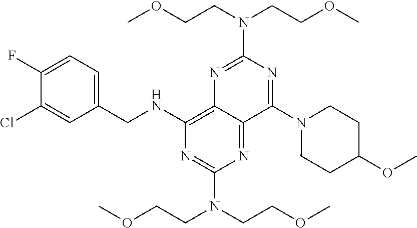 [SMILES: COCCN(CCOC)c1nc(N2CCC(OC)CC2)c2nc(N(CCOC)CCOC)nc(NCc3ccc(F)c(Cl)c3)c2n1] | N4-(3-chloro-4-fluorobenzyl)-N2,N2,N6,N6- tetrakis(2-methoxyethyl)-8-(4- methoxypiperidin-1-yl)pyrimido[5,4- d]pyrimidine-2,4,6-triamine | 665.199 |
| 139 | 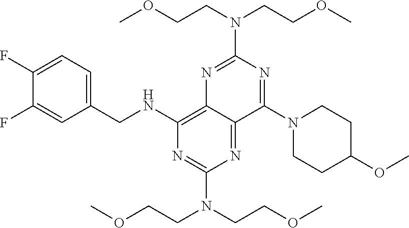 [SMILES: COCCN(CCOC)c1nc(N2CCC(OC)CC2)c2nc(N(CCOC)CCOC)nc(NCc3ccc(F)c(F)c3)c2n1] | N4-(3,4-difluorobenzyl)-N2,N2,N6,N6- tetrakis(2-methoxyethyl)-8-(4- methoxypiperidin-1-yl)pyrimido[5,4- d]pyrimidine-2,4,6-triamine | 648.744 |
| 140 | 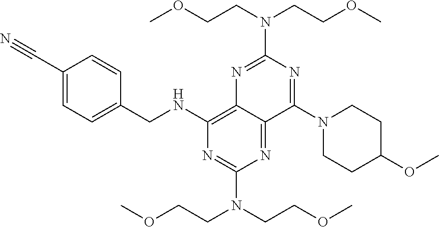 [SMILES: COCCN(CCOC)c1nc(N2CCC(OC)CC2)c2nc(N(CCOC)CCOC)nc(NCc3ccc(C#N)cc3)c2n1] | 4-(((2,6-bis(bis(2-methoxyethyl)amino)-8-(4- methoxypiperidin-1-yl)pyrimido[5,4- d]pyrimidin-4-yl)amino)methyl)benzonitrile | 637.773 |
| 141 | 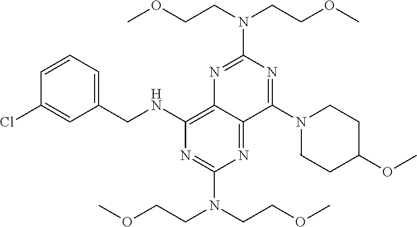 [SMILES: COCCN(CCOC)c1nc(N2CCC(OC)CC2)c2nc(N(CCOC)CCOC)nc(NCc3cccc(Cl)c3)c2n1] | N4-(3-chlorobenzyl)-N2,N2,N6,N6-tetrakis(2- methoxyethyl)-8-(4-methoxypiperidin-1- yl)pyrimido[5,4-d]pyrimidine-2,4,6-triamine | 647.208 |
| 142 | 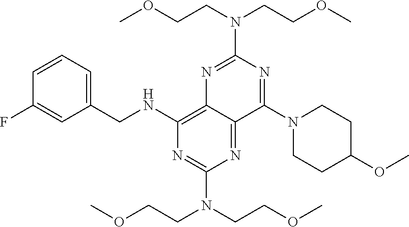 [SMILES: COCCN(CCOC)c1nc(N2CCC(OC)CC2)c2nc(N(CCOC)CCOC)nc(NCc3cccc(F)c3)c2n1] | N4-(3-fluorobenzyl)-N2,N2,N6,N6-tetrakis(2- methoxyethyl)-8-(4-methoxypiperidin-1- yl)pyrimido[5,4-d]pyrimidine-2,4,6-triamine | 630.754 |
| 143 | 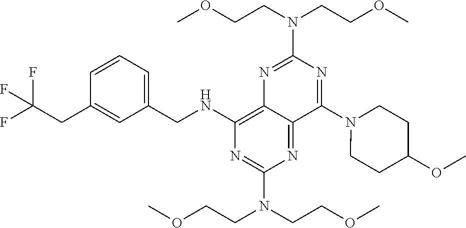 [SMILES: COCCN(CCOC)c1nc(N2CCC(OC)CC2)c2nc(N(CCOC)CCOC)nc(NCc3cccc(CC(F)(F)F)c3)c2n1] | N2,N2,N6,N6-tetrakis(2-methoxyethyl)-8-(4- methoxypiperidin-1-yl)-N4-(3- (trifluoromethoxy)benzyl)pyrimido[5,4- d]pyrimidine-2,4,6-triamine | 696.761 |
| 144 | 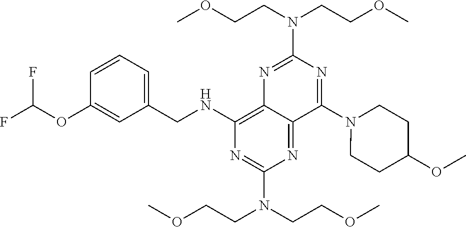 [SMILES: COCCN(CCOC)c1nc(N2CCC(OC)CC2)c2nc(N(CCOC)CCOC)nc(NCc3cccc(OC(F)F)c3)c2n1] | N4-(3-(difluoromethoxy)benzyl)- N2,N2,N6,N6-tetrakis(2-methoxyethyl)-8-(4- methoxypiperidin-1-yl)pyrimido[5,4- d]pyrimidine-2,4,6-triamine | 678.77 |
| 145 | 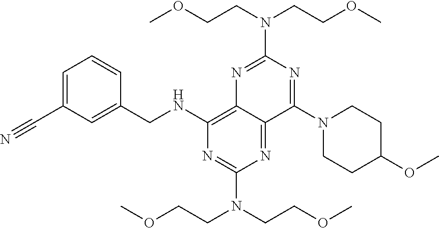 [SMILES: COCCN(CCOC)c1nc(N2CCC(OC)CC2)c2nc(N(CCOC)CCOC)nc(NCc3cccc(C#N)c3)c2n1] | 3-(((2,6-bis(bis(2-methoxyethyl)amino)-8-(4- methoxypiperidin-1-yl)pyrimido[5,4- d]pyrimidin-4-yl)amino)methyl)benzonitrile | 637.773 |
| 146 | 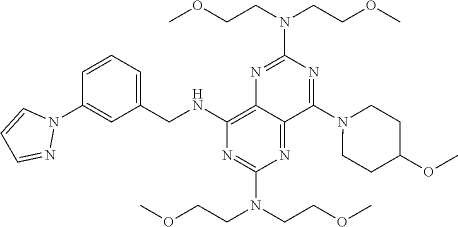 [SMILES: COCCN(CCOC)c1nc(N2CCC(OC)CC2)c2nc(N(CCOC)CCOC)nc(NCc3cccc(-n4cccn4)c3)c2n1] | N4-(3-(1H-pyrazol-1-yl)benzyl)- N2,N2,N6,N6-tetrakis(2-methoxyethyl)-8-(4- methoxypiperidin-1-yl)pyrimido[5,4- d]pyrimidine-2,4,6-triamine | 678.825 |
| 147 | 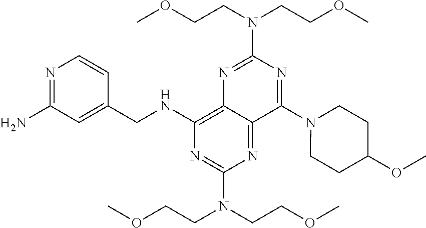 [SMILES: COCCN(CCOC)c1nc(N2CCC(OC)CC2)c2nc(N(CCOC)CCOC)nc(NCc3ccnc(N)c3)c2n1] | N4-((2-aminopyridin-4-yl)methyl)- N2,N2,N6,N6-tetrakis(2-methoxyethyl)-8-(4- methoxypiperidin-1-yl)pyrimido[5,4- d]pyrimidine-2,4,6-triamine | 628.766 |
| 148 | 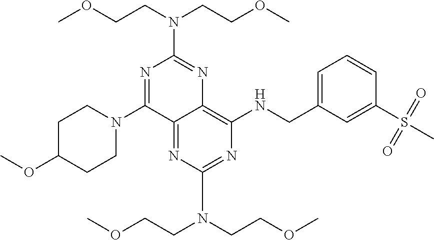 | N2,N2,N6,N6-tetrakis(2-methoxyethyl)-8-(4- methoxypiperidin-1-yl)-N4-(3- (methylsulfonyl)benzyl)pyrimido[5,4- d]pyrimidine-2,4,6-triamine | 690.854 |
| 149 | 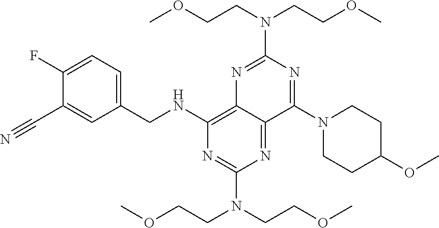 [SMILES: COCCN(CCOC)c1nc(N2CCC(OC)CC2)c2nc(N(CCOC)CCOC)nc(NCc3ccc(F)c(C#N)c3)c2n1] | 5-(((2,6-bis(bis(2-methoxyethyl)amino)-8-(4- methoxypiperidin-1-yl)pyrimido[5,4- d]pyrimidin-4-yl)amino)methyl)-2- fluorobenzonitrile | 655.763 |
| 150 | 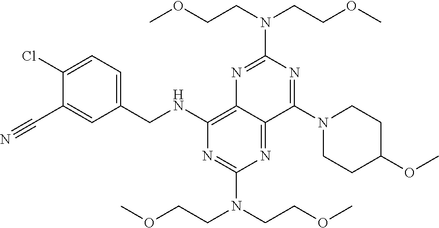 [SMILES: COCCN(CCOC)c1nc(N2CCC(OC)CC2)c2nc(N(CCOC)CCOC)nc(NCc3ccc(Cl)c(C#N)c3)c2n1] | 5-(((2,6-bis(bis(2-methoxyethyl)amino)-8-(4- methoxypiperidin-1-yl)pyrimido[5,4- d]pyrimidin-4-yl)amino)methyl)-2- chlorobenzonitrile | 672.218 |
| 151 | 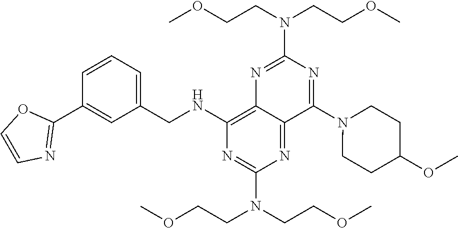 [SMILES: COCCN(CCOC)c1nc(N2CCC(OC)CC2)c2nc(N(CCOC)CCOC)nc(NCc3cccc(-c4ncco4)c3)c2n1] | N2,N2,N6,N6-tetrakis(2-methoxyethyl)-8-(4- methoxypiperidin-1-yl)-N4-(3-(oxazol-2- yl)benzyl)pyrimido[5,4-d]pyrimidine-2,4,6- triamine | 679.81 |
| 152 | 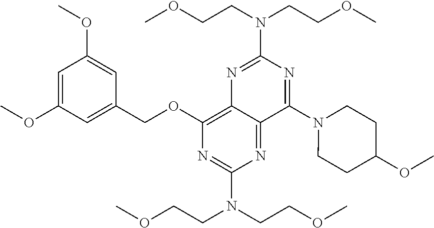 | 4-((3,5-dimethoxybenzyl)oxy)-N2,N2,N6,N6- tetrakis(2-methoxyethyl)-8-(4- methoxypiperidin-1-yl)pyrimido[5,4- d]pyrimidine-2,6-diamine | 673.8 |
| 153 | 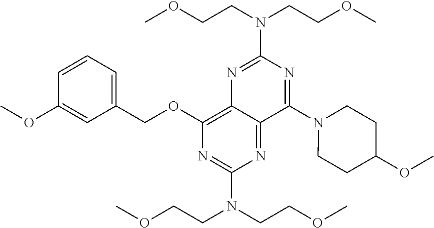 | 4-((3-methoxybenzyl)oxy)-N2,N2,N6,N6- tetrakis(2-methoxyethyl)-8-(4- methoxypiperidin-1-yl)pyrimido[5,4- d]pyrimidine-2,6-diamine | 643.774 |
| 154 | 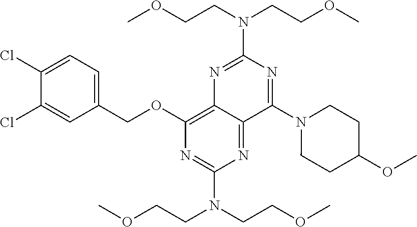 [SMILES: COCCN(CCOC)c1nc(N2CCC(OC)CC2)c2nc(N(CCOC)CCOC)nc(OCc3ccc(Cl)c(Cl)c3)c2n1] | 4-((3,4-dichlorobenzyl)oxy)-N2,N2,N6,N6- tetrakis(2-methoxyethyl)-8-(4- methoxypiperidin-1-yl)pyrimido[5,4- d]pyrimidine-2,6-diamine | 682.638 |
| 155 | 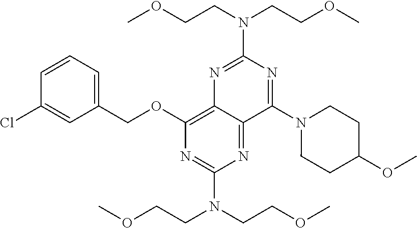 [SMILES: COCCN(CCOC)c1nc(N2CCC(OC)CC2)c2nc(N(CCOC)CCOC)nc(OCc3cccc(Cl)c3)c2n1] | 4-((3-chlorobenzyl)oxy)-N2,N2,N6,N6- tetrakis(2-methoxyethyl)-8-(4- methoxypiperidin-1-yl)pyrimido[5,4- d]pyrimidine-2,6-diamine | 648.193 |
| 156 | 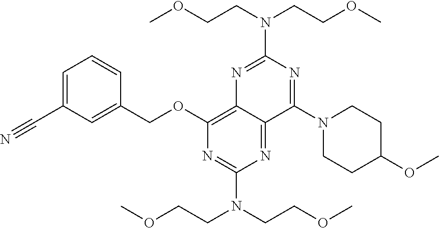 [SMILES: COCCN(CCOC)c1nc(N2CCC(OC)CC2)c2nc(N(CCOC)CCOC)nc(OCc3cccc(C#N)c3)c2n1] | 3-(((2,6-bis(bis(2-methoxyethyl)amino)-8-(4- methoxypiperidin-1-yl)pyrimido[5,4- d]pyrimidin-4-yl)oxy)methyl)benzonitrile | 638.758 |
| 157 | 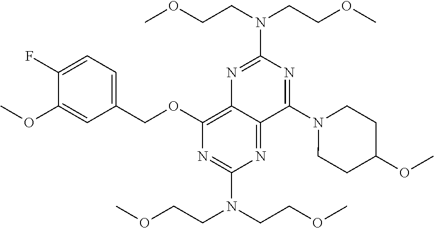 | 4-((4-fluoro-3-methoxybenzyl)oxy)- N2,N2,N6,N6-tetrakis(2-methoxyethyl)-8-(4- methoxypiperidin-1-yl)pyrimido[5,4- d]pyrimidine-2,6-diamine | 661.765 |
| 158 | 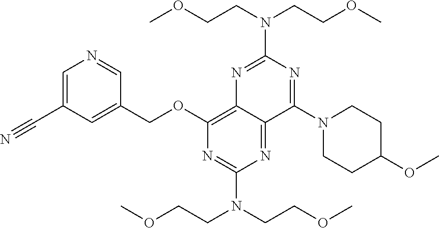 [SMILES: COCCN(CCOC)c1nc(N2CCC(OC)CC2)c2nc(N(CCOC)CCOC)nc(OCc3cncc(C#N)c3)c2n1] | 5-(((2,6-bis(bis(2-methoxyethyl)amino)-8-(4- methoxypiperidin-1-yl)pyrimido[5,4- d]pyrimidin-4-yl)oxy)methyl)nicotinonitrile | 639.746 |
| 159 | 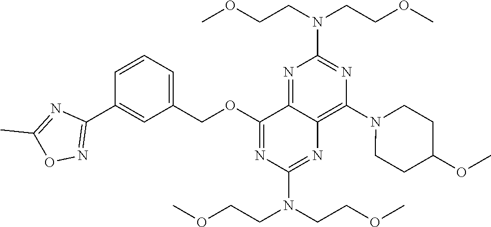 | N2,N2,N6,N6-tetrakis(2-methoxyethyl)-4-(4- methoxypiperidin-1-yl)-8-((3-(5-methyl-1,2,4- oxadiazol-3-yl)benzyl)oxy)pyrimido[5,4- d]pyrimidine-2,6-diamine | 695.809 |
| 160 | 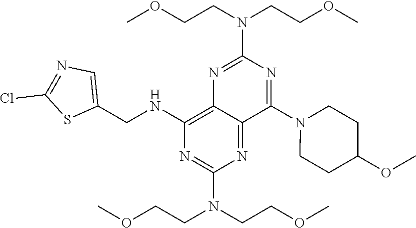 [SMILES: COCCN(CCOC)c1nc(N2CCC(OC)CC2)c2nc(N(CCOC)CCOC)nc(NCc3cnc(Cl)s3)c2n1] | N4-((2-chlorothiazol-5-yl)methyl)- N2,N2,N6,N6-tetrakis(2-methoxyethyl)-8-(4- methoxypiperidin-1-yl)pyrimido[5,4- d]pyrimidine-2,4,6-triamine | 654.224 |
| 161 | 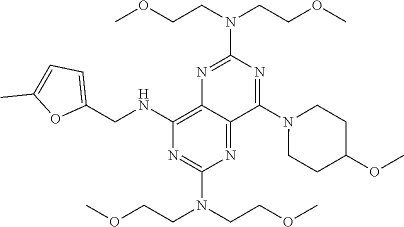 | N2,N2,N6,N6-tetrakis(2-methoxyethyl)-8-(4- methoxypiperidin-1-yl)-N4-((5-methylfuran-2- yl)methyl)pyrimido[5,4-d]pyrimidine-2,4,6- triamine | 616.752 |
| 162 | 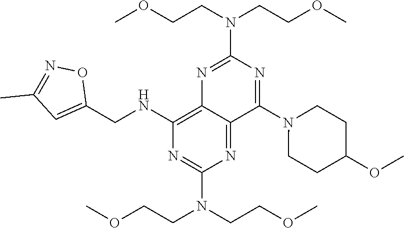 | N2,N2,N6,N6-tetrakis(2-methoxyethyl)-8-(4- methoxypiperidin-1-yl)-N4-((3- methylisoxazol-5-yl)methyl)pyrimido[5,4- d]pyrimidine-2,4,6-triamine | 617.74 |
| 163 | 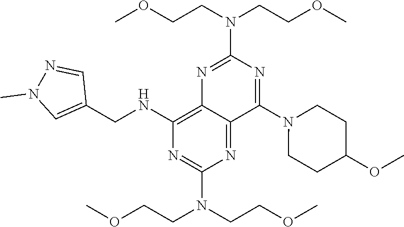 | N4-((1-isopropyl-1H-pyrazol-4-yl)methyl)- N2,N2,N6,N6-tetrakis(2-methoxyethyl)-8-(4- methoxypiperidin-1-yl)pyrimido[5,4- d]pyrimidine-2,4,6-triamine | 644.809 |
| 164 | 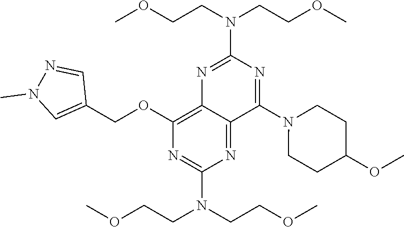 | 4-((1-isopropyl-1H-pyrazol-4-yl)methoxy)- N2,N2,N6,N6-tetrakis(2-methoxyethyl)-8-(4- methoxypiperidin-1-yl)pyrimido[5,4- d]pyrimidine-2,6-diamine | 645.793 |
| 165 | 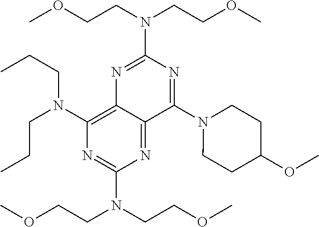 | N2,N2,N6,N6-tetrakis(2-methoxyethyl)-8-(4- methoxypiperidin-1-yl)-N4,N4- dipropylpyrimido[5,4-d]pyrimidine-2,4,6- triamine | 606.8 |
| 166 | 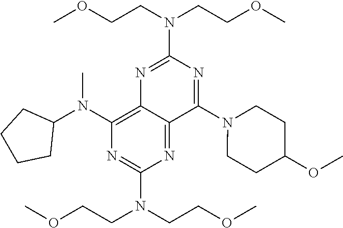 [SMILES: COCCN(CCOC)c1nc(N(C)C2CCCC2)c2nc(N(CCOC)CCOC)nc(N3CCC(OC)CC3)c2n1] | N4-cyclopentyl-N2,N2,N6,N6-tetrakis(2- methoxyethyl)-8-(4-methoxypiperidin-1-yl)- N4-methylpyrimido[5,4-d]pyrimidine-2,4,6- triamine | 604.784 |
| 167 | 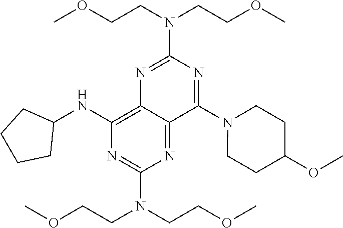 [SMILES: COCCN(CCOC)c1nc(N2CCC(OC)CC2)c2nc(N(CCOC)CCOC)nc(NC3CCCC3)c2n1] | N4-cyclopentyl-N2,N2,N6,N6-tetrakis(2- methoxyethyl)-8-(4-methoxypiperidin-1- yl)pyrimido[5,4-d]pyrimidine-2,4,6-triamine | 590.758 |
| 168 | 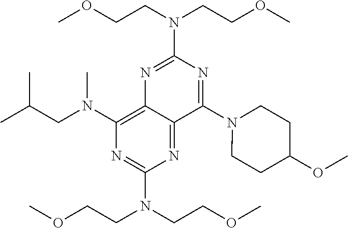 [SMILES: COCCN(CCOC)c1nc(N2CCC(OC)CC2)c2nc(N(CCOC)CCOC)nc(N(C)CC(C)C)c2n1] | N4-isobutyl-N2,N2,N6,N6-tetrakis(2- methoxyethyl)-8-(4-methoxypiperidin-1-yl)- N4-methylpyrimido[5,4-d]pyrimidine-2,4,6- triamine | 592.774 |
| 169 | 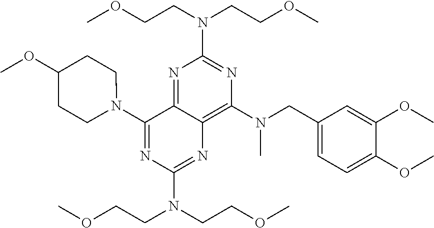 | N4-(3,4-dimethoxybenzyl)-N2,N2,N6,N6- tetrakis(2-methoxyethyl)-8-(4- methoxypiperidin-1-yl)-N4- methylpyrimido[5,4-d]pyrimidine-2,4,6- triamine | 686.842 |
| 170 | 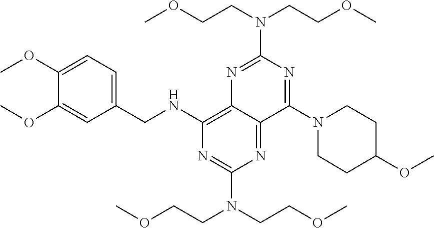 | N4-(3,4-dimethoxybenzyl)-N2,N2,N6,N6- tetrakis(2-methoxyethyl)-8-(4- methoxypiperidin-1-yl)pyrimido[5,4- d]pyrimidine-2,4,6-triamine | 672.815 |
| 171 | 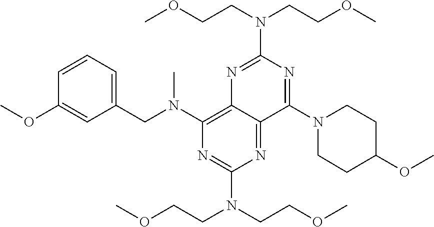 | N4-(3-methoxybenzyl)-N2,N2,N6,N6- tetrakis(2-methoxyethyl)-8-(4- methoxypiperidin-1-yl)-N4- methylpyrimido[5,4-d]pyrimidine-2,4,6- triamine | 656.816 |
| 172 | 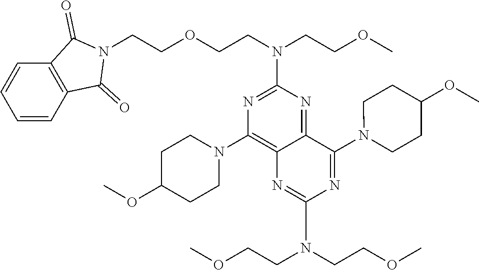 [SMILES: COCCN(CCOC)c1nc(N2CCC(OC)CC2)c2nc(N(CCOC)CCOCCN3C(=O)c4ccccc4C3=O)nc(N3CCC(OC)CC3)c2n1] | 2-(2-(2-((6-(bis(2-methoxyethyl)amino)-4,8- bis(4-methoxypiperidin-1-yl)pyrimido[5,4- d]pyrimidin-2-yl)(2- methoxyethyl)amino)ethoxy)ethyl)isoindoline- 1,3-dione | 779.925 |
| 173 | 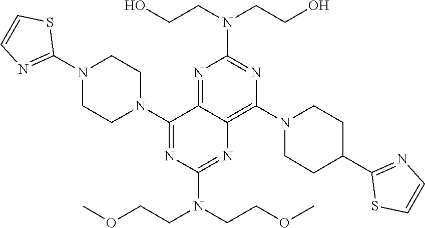 [SMILES: COCCN(CCOC)c1nc(N2CCN(c3nccs3)CC2)c2nc(N(CCO)CCO)nc(N3CCC(c4nccs4)CC3)c2n1] | 2,2′-((6-(bis(2-methoxyethyl)amino)-4,8-bis(4- (thiazol-2-yl)piperazin-1-yl)pyrimido[5,4- d]pyrimidin-2-yl)azanediyl)diethanol | 700.878 |
| 174 | 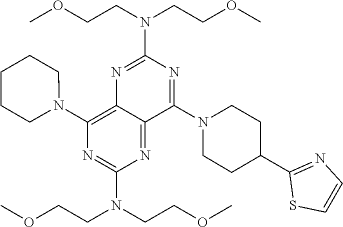 [SMILES: COCCN(CCOC)c1nc(N2CCC(c3nccs3)CC2)c2nc(N(CCOC)CCOC)nc(N3CCCCC3)c2n1] | N2,N2,N6,N6-tetrakis(2-methoxyethyl)-4- (piperidin-1-yl)-8-(4-(thiazol-2-yl)piperazin-1- yl)pyrimido[5,4-d]pyrimidine-2,6-diamine | 644.832 |
| 175 | 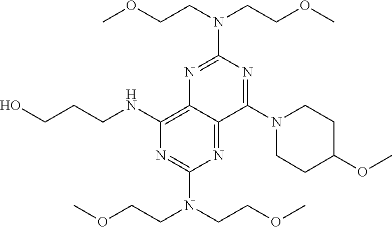 [SMILES: COCCN(CCOC)c1nc(N2CCC(OC)CC2)c2nc(N(CCOC)CCOC)nc(NCCCO)c2n1] | 3-((2,6-bis(bis(2-methoxyethyl)amino)-8-(4- methoxypiperidin-1-yl)pyrimido[5,4- d]pyrimidin-4-yl)amino)propan-1-ol | 580.72 |
| 176 | 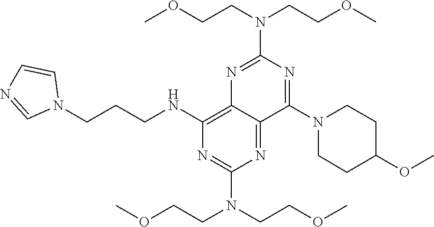 [SMILES: COCCN(CCOC)c1nc(N2CCC(OC)CC2)c2nc(N(CCOC)CCOC)nc(NCCCn3ccnc3)c2n1] | N4-(3-(1H-imidazol-1-yl)propyl)- N2,N2,N6,N6-tetrakis(2-methoxyethyl)-8-(4- methoxypiperidin-1-yl)pyrimido[5,4- d]pyrimidine-2,4,6-triamine | 630.782 |
| 177 | 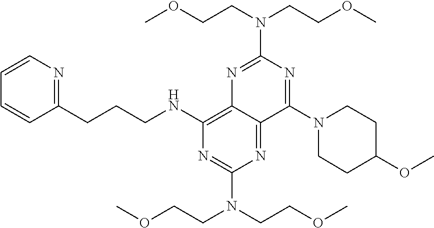 [SMILES: COCCN(CCOC)c1nc(N2CCC(OC)CC2)c2nc(N(CCOC)CCOC)nc(NCCCc3ccccn3)c2n1] | N2,N2,N6,N6-tetrakis(2-methoxyethyl)-8-(4- methoxypiperidin-1-yl)-N4-(3-(pyridin-2- yl)propyl)pyrimido[5,4-d]pyrimidine-2,4,6- triamine | 641.805 |
| 178 | 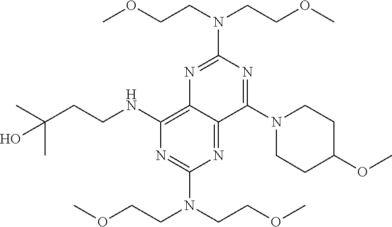 [SMILES: COCCN(CCOC)c1nc(N2CCC(OC)CC2)c2nc(N(CCOC)CCOC)nc(NCCC(C)(C)O)c2n1] | 4-((2,6-bis(bis(2-methoxyethyl)amino)-8-(4- methoxypiperidin-1-yl)pyrimido[5,4- d]pyrimidin-4-yl)amino)-2-methylbutan-2-ol | 608.773 |
| 179 | 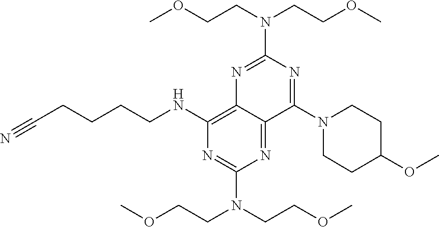 [SMILES: COCCN(CCOC)c1nc(N2CCC(OC)CC2)c2nc(N(CCOC)CCOC)nc(NCCCCC#N)c2n1] | 5-((2,6-bis(bis(2-methoxyethyl)amino)-8-(4- methoxypiperidin-1-yl)pyrimido[5,4- d]pyrimidin-4-yl)amino)pentanenitrile | 603.757 |
| 180 | 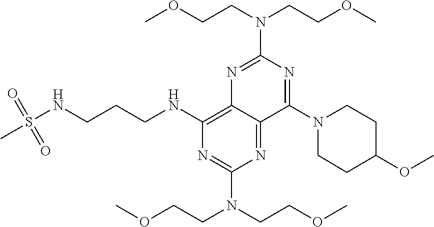 | N-(3-((2,6-bis(bis(2-methoxyethyl)amino)-8- (4-methoxypiperidin-1-yl)pyrimido[5,4- d]pyrimidin-4- yl)amino)propyl)methanesulfonamide | 657.826 |
| 181 | 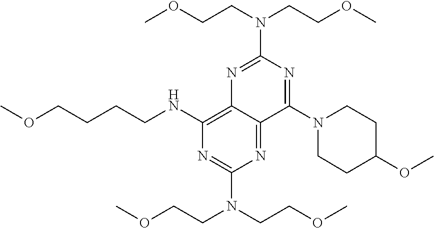 | N4-(4-methoxybutyl)-N2,N2,N6,N6-tetrakis(2- methoxyethyl)-8-(4-methoxypiperidin-1- yl)pyrimido[5,4-d]pyrimidine-2,4,6-triamine | 608.773 |
| 182 | 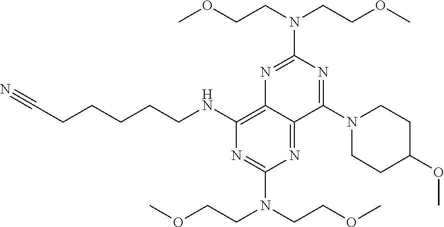 [SMILES: COCCN(CCOC)c1nc(N2CCC(OC)CC2)c2nc(N(CCOC)CCOC)nc(NCCCCCC#N)c2n1] | 6-((2,6-bis(bis(2-methoxyethyl)amino)-8-(4- methoxypiperidin-1-yl)pyrimido[5,4- d]pyrimidin-4-yl)amino)hexanenitrile | 617.783 |
| 183 | 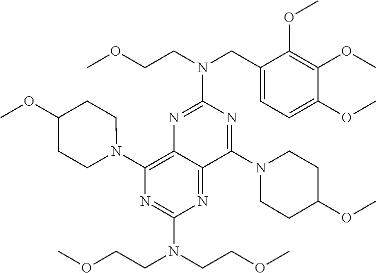 | N2,N2,N6-tris(2-methoxyethyl)-4,8-bis(4- methoxypiperidin-1-yl)-N6-(2,3,4- trimethoxybenzyl)pyrimido[5,4-d]pyrimidine- 2,6-diamine | 742.905 |
| 184 | 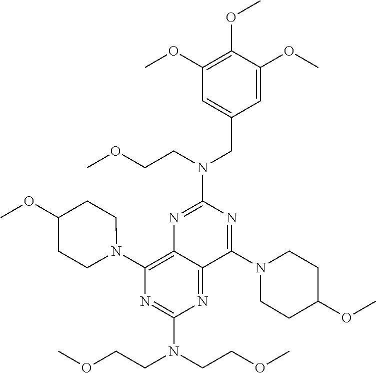 | N2,N2,N6-tris(2-methoxyethyl)-4,8-bis(4- methoxypiperidin-1-yl)-N6-(3,4,5- trimethoxybenzyl)pyrimido[5,4-d]pyrimidine- 2,6-diamine | 742.905 |
| 185 | 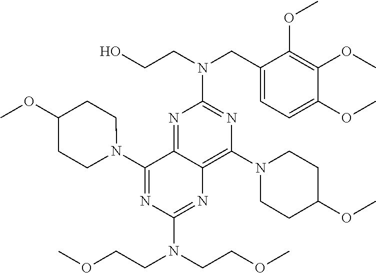 | 2-((6-(bis(2-methoxyethyl)amino)-4,8-bis(4- methoxypiperidin-1-yl)pyrimido[5,4- d]pyrimi<din-2-yl)(2,3,4- trimethoxybenzyl)amino)ethanol | 728.879 |
| 186 | 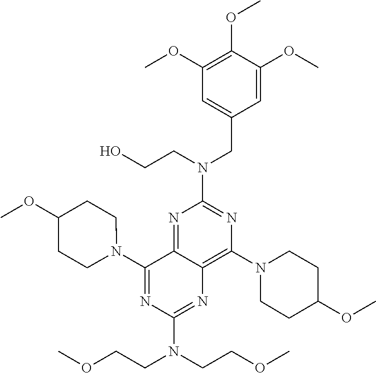 | 2-((6-(bis(2-methoxyethyl)amino)-4,8-bis(4- methoxypiperidin-1-yl)pyrimido[5,4- d]pyrimidin-2-yl)(3,4,5- trimethoxybenzyl)amino)ethanol | 728.879 |
| 187 | 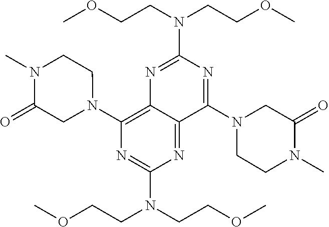 [SMILES: COCCN(CCOC)c1nc(N2CCN(C)C(=O)C2)c2nc(N(CCOC)CCOC)nc(N3CCN(C)C(=O)C3)c2n1] | 4,4′-(2,6-bis(bis(2- methoxyethyl)amino)pyrimido[5,4- d]pyrimidine-4,8-diyl)bis(1-methylpiperazin-2- one) | 618.728 |
| 188 | 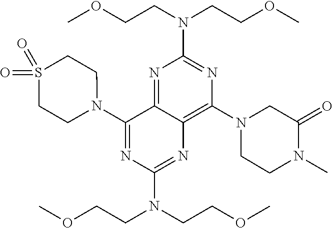 [SMILES: COCCN(CCOC)c1nc(N2CCN(C)C(=O)C2)c2nc(N(CCOC)CCOC)nc(N3CCS(=O)(=O)CC3)c2n1] | 4-(2,6-bis(bis(2-methoxyethyl)amino)-8-(1,1- dioxidothiomorpholino)pyrimido[5,4- d]pyrimidin-4-yl)-1-methylpiperazin-2-one | 639.767 |
| 189 | 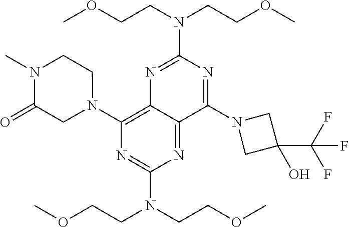 [SMILES: COCCN(CCOC)c1nc(N2CC(O)(C(F)(F)F)C2)c2nc(N(CCOC)CCOC)nc(N3CCN(C)C(=O)C3)c2n1] | 4-(2,6-bis(bis(2-methoxyethyl)amino)-8-(3- hydroxy-3-(trifluoromethyl)azetidin-1- yl)pyrimido[5,4-d]pyrimidin-4-yl)-1- methylpiperazin-2-one | 645.674 |
| 190 | 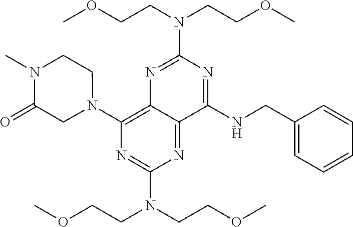 [SMILES: COCCN(CCOC)c1nc(N2CCN(C)C(=O)C2)c2nc(N(CCOC)CCOC)nc(NCc3ccccc3)c2n1] | 4-(8-(benzylamino)-2,6-bis(bis(2- methoxyethyl)amino)pyrimido[5,4- d]pyrimidin-4-yl)-1-methylpiperazin-2-one | 611.736 |
| 191 | 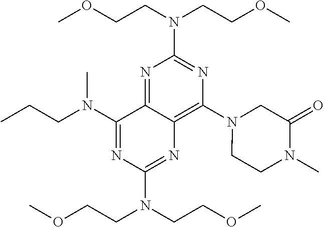 [SMILES: CCCN(C)c1nc(N(CCOC)CCOC)nc2c(N3CCN(C)C(=O)C3)nc(N(CCOC)CCOC)nc12] | 4-(2,6-bis(bis(2-methoxyethyl)amino)-8- (methyl(propyl)amino)pyrimido[5,4- d]pyrimidin-4-yl)-1-methylpiperazin-2-one | 577.719 |
| 192 | 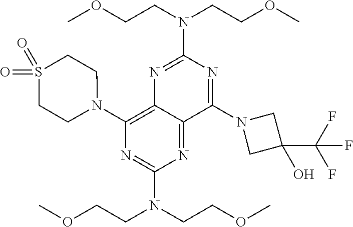 [SMILES: COCCN(CCOC)c1nc(N2CC(O)(C(F)(F)F)C2)c2nc(N(CCOC)CCOC)nc(N3CCS(=O)(=O)CC3)c2n1] | 4-(2,6-bis(bis(2-methoxyethyl)amino)-8-(3- hydroxy-3-(trifluoromethyl)azetidin-1- yl)pyrimido[5,4-d]pyrimidin-4- yl)thiomorpholine 1,1-dioxide | 666.713 |
| 193 | 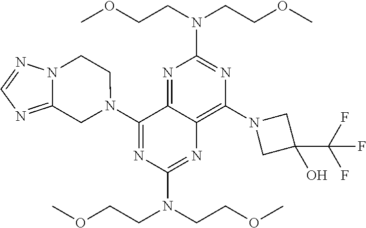 [SMILES: COCCN(CCOC)c1nc(N2CC(O)(C(F)(F)F)C2)c2nc(N(CCOC)CCOC)nc(N3CCn4ncnc4C3)c2n1] | 1-(2,6-bis(bis(2-methoxyethyl)amino)-8-(5,6- dihydro-[1,2,4]triazolo[1,5-a]pyrazin-7(8H)- yl)pyrimido[5,4-d]pyrimidin-4-yl)-3- (trifluoromethyl)azetidin-3-ol | 655.672 |
| 194 | 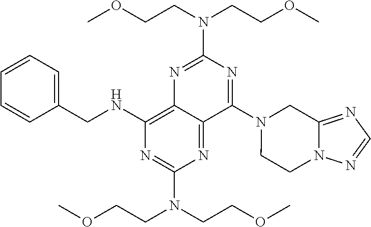 [SMILES: COCCN(CCOC)c1nc(N2CCn3ncnc3C2)c2nc(N(CCOC)CCOC)nc(NCc3ccccc3)c2n1] | N4-benzyl-8-(5,6-dihydro-[1,2,4]triazolo[1,5- a]pyrazin-7(8H)-yl)-N2,N2,N6,N6-tetrakis(2- methoxyethyl)pyrimido[5,4-d]pyrimidine- 2,4,6-triamine | 621.734 |
| 195 | 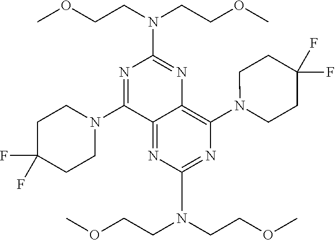 [SMILES: COCCN(CCOC)c1nc(N2CCC(F)(F)CC2)c2nc(N(CCOC)CCOC)nc(N3CCC(F)(F)CC3)c2n1] | 4,8-bis(4,4-difluoropiperidin-1-yl)- N2,N2,N6,N6-tetrakis(2- methoxyethyl)pyrimido[5,4-d]pyrimidine-2,6- diamine | 632.705 |
| 196 | 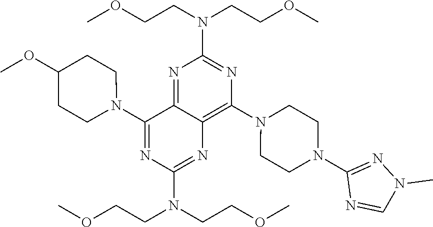 | N2,N2,N6,N6-tetrakis(2-methoxyethyl)-4-(4- methoxypiperidin-1-yl)-8-(4-(1-methyl-1H- 1,2,4-triazol-3-yl)piperazin-1-yl)pyrimido[5,4- d]pyrimidine-2,6-diamine | 672.836 |
| 197 | 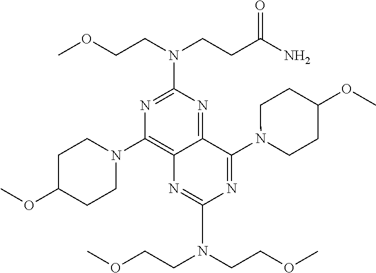 | 3-((6-(bis(2-methoxyethyl)amino)-4,8-bis(4- methoxypiperidin-1-yl)pyrimido[5,4- d]pyrimidin-2-yl)(2- methoxyethyl)amino)propanamide | 633.795 |
| 198 | 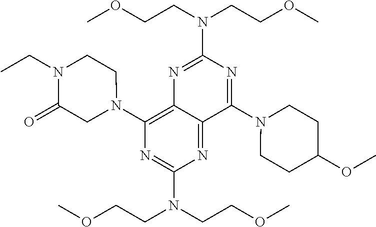 [SMILES: CCN1CCN(c2nc(N(CCOC)CCOC)nc3c(N4CCC(OC)CC4)nc(N(CCOC)CCOC)nc23)CC1=O] | 4-(2,6-bis(bis(2-methoxyethyl)amino)-8-(4- methoxypiperidin-1-yl)pyrimido[5,4- d]pyrimidin-4-yl)-1-ethylpiperazin-2-one | 633.795 |
| 199 | 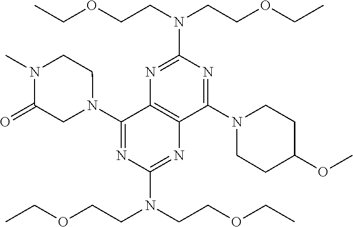 [SMILES: CCOCCN(CCOCC)c1nc(N2CCN(C)C(=O)C2)c2nc(N(CCOCC)CCOCC)nc(N3CCC(OC)CC3)c2n1] | 4-(2,6-bis(bis(2-ethoxyethyl)amino)-8-(4- methoxypiperidin-1-yl)pyrimido[5,4- d]pyrimidin-4-yl)-1-methylpiperazin-2-one | 675.876 |
| 200 | 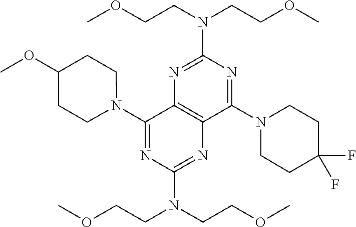 [SMILES: COCCN(CCOC)c1nc(N2CCC(F)(F)CC2)c2nc(N(CCOC)CCOC)nc(N3CCC(OC)CC3)c2n1] | 4-(4,4-difluoropiperidin-1-yl)-N2,N2,N6,N6- tetrakis(2-methoxyethyl)-8-(4- methoxypiperidin-1-yl)pyrimido[5,4- d]pyrimidine-2,6-diamine | 626.750 |
| 201 | 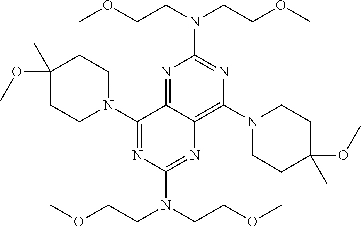 | 4,8-bis(4-methoxy-4-methylpiperidin-1-yl)- N2,N2,N6,N6-tetrakis(2- methoxyethyl)pyrimido[5,4-d]pyrimidine-2,6- diamine | 648.850 |
| 202 | 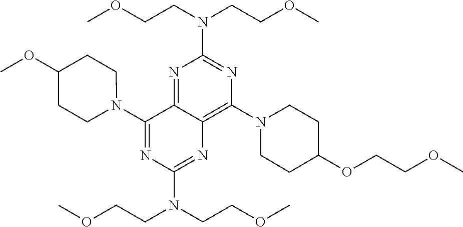 | 4-(4-(2-methoxyethoxy)piperidin-1-yl)- N2,N2,N6,N6-tetrakis(2-methoxyethyl)-8-(4- methoxypiperidin-1-yl)pyrimido[5,4- d]pyrimidine-2,6-diamine | 664.849 |
| 203 | 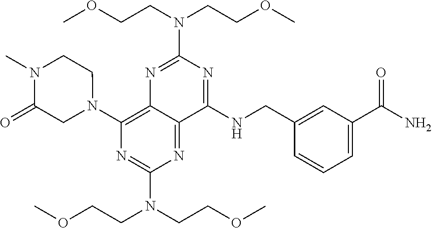 [SMILES: COCCN(CCOC)c1nc(N2CCN(C)C(=O)C2)c2nc(N(CCOC)CCOC)nc(NCc3cccc(C(N)=O)c3)c2n1] | 3-(((2,6-bis(bis(2-methoxyethyl)amino)-8-(4- methyl-3-oxopiperazin-1-yl)pyrimido[5,4- d]pyrimidin-4-yl)amino)methyl)benzamide | 654.773 |
| 204 | 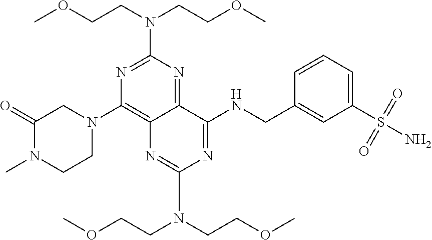 [SMILES: COCCN(CCOC)c1nc(N2CCN(C)C(=O)C2)c2nc(N(CCOC)CCOC)nc(NCc3cccc(S(N)(=O)=O)c3)c2n1] | 3-(((2,6-bis(bis(2-methoxyethyl)amino)-8-(4- methyl-3-oxopiperazin-1-yl)pyrimido[5,4- d]pyrimidin-4- yl)amino)methyl)benzenesulfonamide | 690.821 |
| 205 | 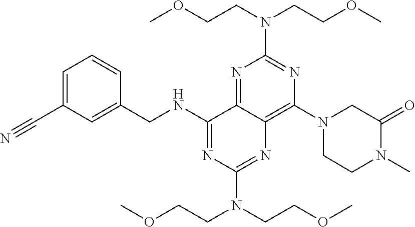 [SMILES: COCCN(CCOC)c1nc(N2CCN(C)C(=O)C2)c2nc(N(CCOC)CCOC)nc(NCc3cccc(C#N)c3)c2n1] | 3-(((2,6-bis(bis(2-methoxyethyl)amino)-8-(4- methyl-3-oxopiperazin-1-yl)pyrimido[5,4- d]pyrimidin-4-yl)amino)methyl)benzonitrile | 636.758 |
| 206 | 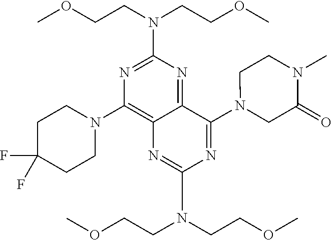 [SMILES: COCCN(CCOC)c1nc(N2CCN(C)C(=O)C2)c2nc(N(CCOC)CCOC)nc(N3CCC(F)(F)CC3)c2n1] | 4-(2,6-bis(bis(2-methoxyethyl)amino)-8-(4,4- difluoropiperidin-1-yl)pyrimido[5,4- d]pyrimidin-4-yl)-1-methylpiperazin-2-one | 625.722 |
| 207 | 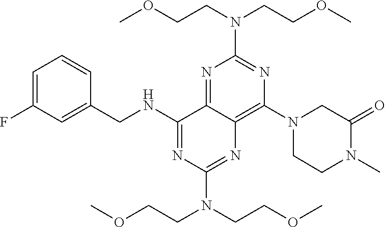 [SMILES: COCCN(CCOC)c1nc(N2CCN(C)C(=O)C2)c2nc(N(CCOC)CCOC)nc(NCc3cccc(F)c3)c2n1] | 4-(2,6-bis(bis(2-methoxyethyl)amino)-8-((3- fluorobenzyl)amino)pyrimido[5,4-d]pyrimidin- 4-yl)-1-methylpiperazin-2-one | 629.738 |
| 208 | 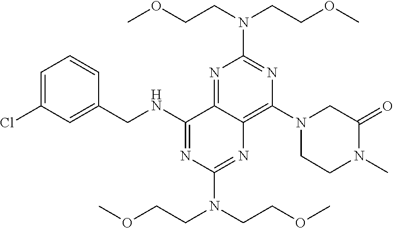 [SMILES: COCCN(CCOC)c1nc(N2CCN(C)C(=O)C2)c2nc(N(CCOC)CCOC)nc(NCc3cccc(Cl)c3)c2n1] | 4-(2,6-bis(bis(2-methoxyethyl)amino)-8-((3- chlorobenzyl)amino)pyrimido[5,4- d]pyrimidin-4-yl)-1-methylpiperazin-2-one | 646.190 |
| 209 | 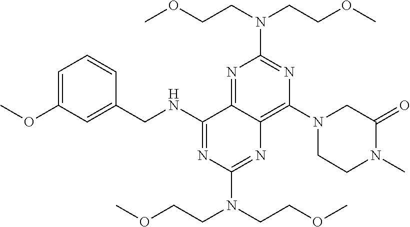 [SMILES: COCCN(CCOC)c1nc(N2CCN(C)C(=O)C2)c2nc(N(CCOC)CCOC)nc(NCc3cccc(OC)c3)c2n1] | 4-(2,6-bis(bis(2-methoxyethyl)amino)-8-((3- methoxybenzyl)amino)pyrimido[5,4- d]pyrimidin-4-yl)-1-methylpiperazin-2-one | 641.774 |
| 210 | 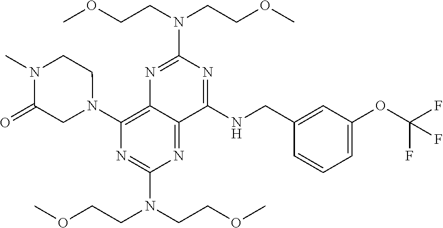 [SMILES: COCCN(CCOC)c1nc(N2CCN(C)C(=O)C2)c2nc(N(CCOC)CCOC)nc(NCc3cccc(OC(F)(F)F)c3)c2n1] | 4-(2,6-bis(bis(2-methoxyethyl)amino)-8-((3- (trifluoromethoxy)benzyl)amino)pyrimido[5,4- d]pyrimidin-4-yl)-1-methylpiperazin-2-one | 695.745 |
| 211 | 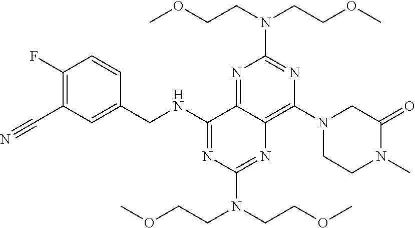 [SMILES: COCCN(CCOC)c1nc(N2CCN(C)C(=O)C2)c2nc(N(CCOC)CCOC)nc(NCc3ccc(F)c(C#N)c3)c2n1] | 5-(((2,6-bis(bis(2-methoxyethyl)amino)-8-(4- methyl-3-oxopiperazin-1-yl)pyrimido[5,4- d]pyrimidin-4-yl)amino)methyl)-2- fluorobenzonitrile | 654.748 |
| 212 | 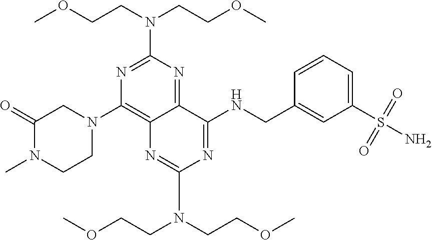 [SMILES: COCCN(CCOC)c1nc(N2CCN(C)C(=O)C2)c2nc(N(CCOC)CCOC)nc(NCc3cccc(S(N)(=O)=O)c3)c2n1] | 4-(2,6-bis(bis(2-methoxyethyl)amino)-8-((3- (methylsulfonyl)benzyl)amino)pyrimido[5,4- d]pyrimidin-4-yl)-1-methylpiperazin-2-one | 689.833 |
| 213 | 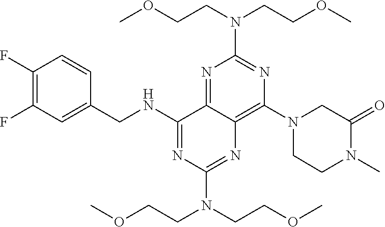 [SMILES: COCCN(CCOC)c1nc(N2CCN(C)C(=O)C2)c2nc(N(CCOC)CCOC)nc(NCc3ccc(F)c(F)c3)c2n1] | 4-(2,6-bis(bis(2-methoxyethyl)amino)-8-((3,4- difluorobenzyl)amino)pyrimido[5,4- d]pyrimidin-4-yl)-1-methylpiperazin-2-one | 647.728 |
| 214 | 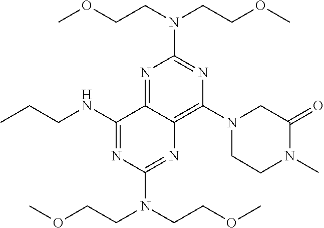 [SMILES: CCCNc1nc(N(CCOC)CCOC)nc2c(N3CCN(C)C(=O)C3)nc(N(CCOC)CCOC)nc12] | 4-(2,6-bis(bis(2-methoxyethyl)amino)-8- (propylamino)pyrimido[5,4-d]pyrimidin-4-yl)- 1-methylpiperazin-2-one | 563.704 |
| 215 | 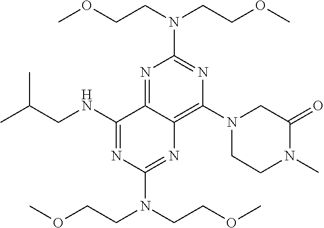 [SMILES: COCCN(CCOC)c1nc(N2CCN(C)C(=O)C2)c2nc(N(CCOC)CCOC)nc(NCC(C)C)c2n1] | 4-(2,6-bis(bis(2-methoxyethyl)amino)-8- (isobutylamino)pyrimido[5,4-d]pyrimidin-4- yl)-1-methylpiperazin-2-one | 577.731 |
| 216 | 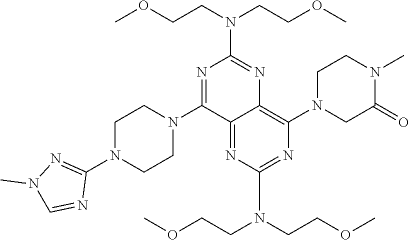 [SMILES: COCCN(CCOC)c1nc(N2CCN(C)C(=O)C2)c2nc(N(CCOC)CCOC)nc(N3CCN(c4ncn(C)n4)CC3)c2n1] | 4-(2,6-bis(bis(2-methoxyethyl)amino)-8-(4-(1- methyl-1H-1,2,4-triazol-3-yl)piperazin-1- yl)pyrimido[5,4-d]pyrimidin-4-yl)-1- methylpiperazin-2-one | 671.808 |
| 217 | 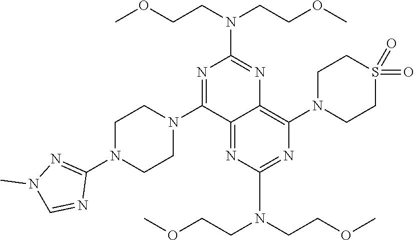 [SMILES: COCCN(CCOC)c1nc(N2CCS(=O)(=O)CC2)c2nc(N(CCOC)CCOC)nc(N3CCN(c4ncn(C)n4)CC3)c2n1] | 4-(2,6-bis(bis(2-methoxyethyl)amino)-8-(4-(1- methyl-1H-1,2,4-triazol-3-yl)piperazin-1- yl)pyrimido[5,4-d]pyrimidin-4- yl)thiomorpholine 1,1-dioxide | 692.841 |
| 218 | 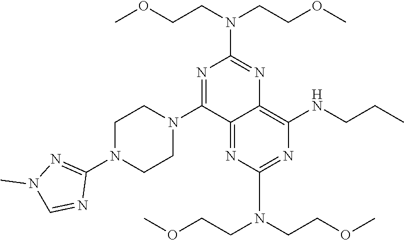 [SMILES: CCCNc1nc(N(CCOC)CCOC)nc2c(N3CCN(c4ncn(C)n4)CC3)nc(N(CCOC)CCOC)nc12] | N2,N2,N6,N6-tetrakis(2-methoxyethyl)-8-(4- (1-methyl-1H-1,2,4-triazol-3-yl)piperazin-1- yl)-N4-propylpyrimido[5,4-d]pyrimidine- 2,4,6-triamine | 616.772 |
| 219 | 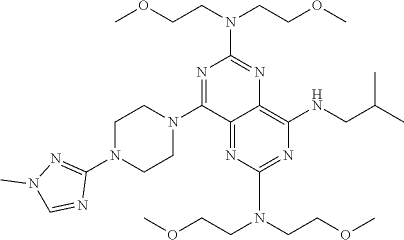 [SMILES: COCCN(CCOC)c1nc(N2CCN(c3ncn(C)n3)CC2)c2nc(N(CCOC)CCOC)nc(NCC(C)C)c2n1] | N4-isobutyl-N2,N2,N6,N6-tetrakis(2- methoxyethyl)-8-(4-(1-methyl-1H-1,2,4-triazol- 3-yl)piperazin-1-yl)pyrimido[5,4- d]pyrimidine-2,4,6-triamine | 630.799 |
| 220 | 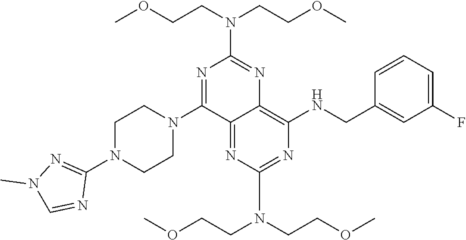 [SMILES: COCCN(CCOC)c1nc(N2CCN(c3ncn(C)n3)CC2)c2nc(N(CCOC)CCOC)nc(NCc3cccc(F)c3)c2n1] | N4-(3-fluorobenzyl)-N2,N2,N6,N6-tetrakis(2- methoxyethyl)-8-(4-(1-methyl-1H-1,2,4- triazol-3-yl)piperazin-1-yl)pyrimido[5,4- d]pyrimidine-2,4,6-triamine | 682.806 |
| 221 | 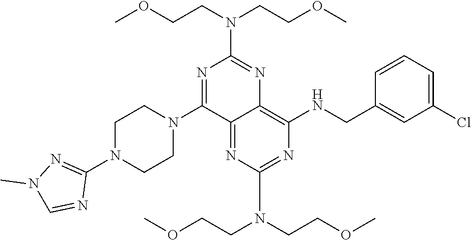 [SMILES: COCCN(CCOC)c1nc(N2CCN(c3ncn(C)n3)CC2)c2nc(N(CCOC)CCOC)nc(NCc3cccc(Cl)c3)c2n1] | N4-(3-chlorobenzyl)-N2,N2,N6,N6-tetrakis(2- methoxyethyl)-8-(4-(1-methyl-1H-1,2,4- triazol-3-yl)piperazin-1-yl)pyrimido[5,4- d]pyrimidine-2,4,6-triamine | 699.258 |
| 222 | 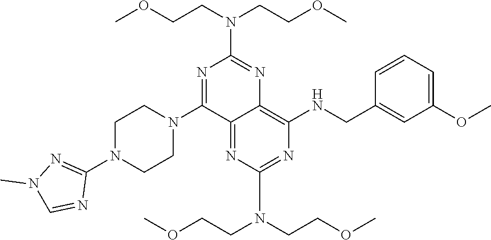 | N4-(3-methoxybenzyl)-N2,N2,N6,N6- tetrakis(2-methoxyethyl)-8-(4-(1-methyl-1H- 1,2,4-triazol-3-yl)piperazin-1-yl)pyrimido[5,4- d]pyrimidine-2,4,6-triamine | 694.842 |
| 223 | 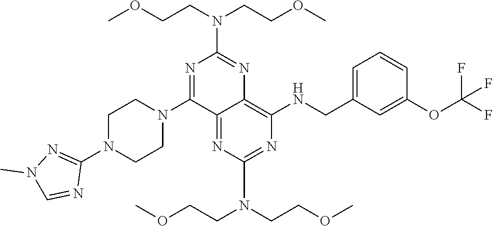 [SMILES: COCCN(CCOC)c1nc(N2CCN(c3ncn(C)n3)CC2)c2nc(N(CCOC)CCOC)nc(NCc3cccc(OC(F)(F)F)c3)c2n1] | N2,N2,N6,N6-tetrakis(2-methoxyethyl)-8-(4- (1-methyl-1H-1,2,4-triazol-3-yl)piperazin-1- yl)-N4-(3- (trifluoromethoxy)benzyl)pyrimido[5,4- d]pyrimidine-2,4,6-triamine | 748.813 |
| 224 | 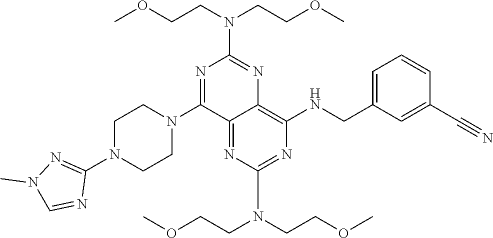 [SMILES: COCCN(CCOC)c1nc(N2CCN(c3ncn(C)n3)CC2)c2nc(N(CCOC)CCOC)nc(NCc3cccc(C#N)c3)c2n1] | 3-(((2,6-bis(bis(2-methoxyethyl)amino)-8-(4- (1-methyl-1H-1,2,4-triazol-3-yl)piperazin-1- yl)pyrimido[5,4-d]pyrimidin-4- yl)amino)methyl)benzonitrile | 689.826 |
| 225 | 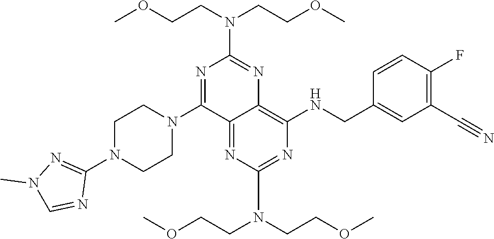 [SMILES: COCCN(CCOC)c1nc(N2CCN(c3ncn(C)n3)CC2)c2nc(N(CCOC)CCOC)nc(NCc3ccc(F)c(C#N)c3)c2n1] | 5-(((2,6-bis(bis(2-methoxyethyl)amino)-8-(4- (1-methyl-1H-1,2,4-triazol-3-yl)piperazin-1- yl)pyrimido[5,4-d]pyrimidin-4- yl)amino)methyl)-2-fluorobenzonitrile | 707.816 |
| 226 | 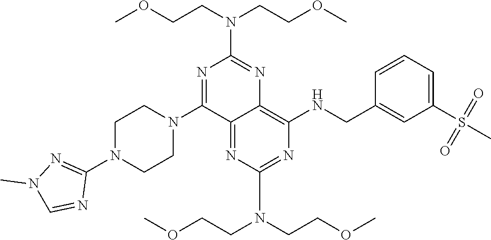 | N2,N2,N6,N6-tetrakis(2-methoxyethyl)-8-(4- (1-methyl-1H-1,2,4-triazol-3-yl)piperazin-1- yl)-N4-(3- (methylsulfonyl)benzyl)pyrimido[5,4- d]pyrimidine-2,4,6-triamine | 742.901 |
| 227 | 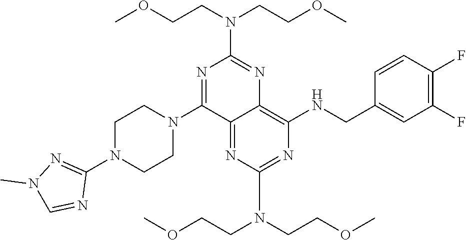 [SMILES: COCCN(CCOC)c1nc(N2CCN(c3ncn(C)n3)CC2)c2nc(N(CCOC)CCOC)nc(NCc3ccc(F)c(F)c3)c2n1] | N4-(3,4-difluorobenzyl)-N2,N2,N6,N6- tetrakis(2-methoxyethyl)-8-(4-(1-methyl-1H- 1,2,4-triazol-3-yl)piperazin-1-yl)pyrimido[5,4- d]pyrimidine-2,4,6-triamine | 700.796 |
| 228 | 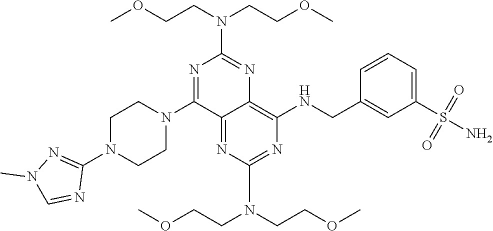 [SMILES: COCCN(CCOC)c1nc(N2CCN(c3ncn(C)n3)CC2)c2nc(N(CCOC)CCOC)nc(NCc3cccc(S(N)(=O)=O)c3)c2n1] | 3-(((2,6-bis(bis(2-methoxyethyl)amino)-8-(4- (1-methyl-1H-1,2,4-triazol-3-yl)piperazin-1- yl)pyrimido[5,4-d]pyrimidin-4- yl)amino)methyl)benzenesulfonamide | 743.889 |
| 229 | 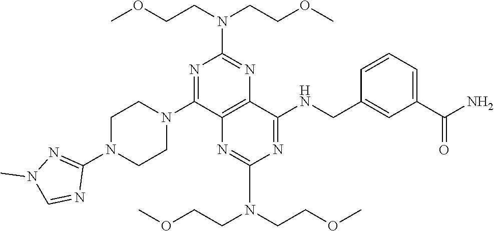 [SMILES: COCCN(CCOC)c1nc(N2CCN(c3ncn(C)n3)CC2)c2nc(N(CCOC)CCOC)nc(NCc3cccc(C(N)=O)c3)c2n1] | 3-(((2,6-bis(bis(2-methoxyethyl)amino)-8-(4- (1-methyl-1H-1,2,4-triazol-3-yl)piperazin-1- yl)pyrimido[5,4-d]pyrimidin-4- yl)amino)methyl)benzamide | 707.841 |
| 230 | 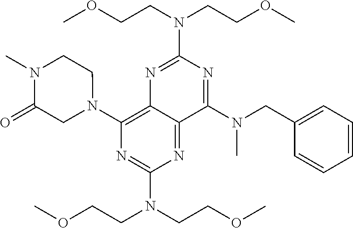 [SMILES: COCCN(CCOC)c1nc(N2CCN(C)C(=O)C2)c2nc(N(CCOC)CCOC)nc(N(C)Cc3ccccc3)c2n1] | 4-(8-(benzyl(methyl)amino)-2,6-bis(bis(2- methoxyethyl)amino)pyrimido[5,4- d]pyrimidin-4-yl)-1-methylpiperazin-2-one | 625.775 |
| 231 | 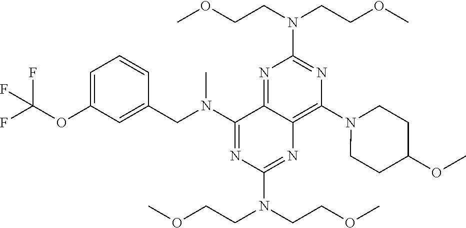 [SMILES: COCCN(CCOC)c1nc(N2CCC(OC)CC2)c2nc(N(CCOC)CCOC)nc(N(C)Cc3cccc(OC(F)(F)F)c3)c2n1] | N2,N2,N6,N6-tetrakis(2-methoxyethyl)-8-(4- methoxypiperidin-1-yl)-N4-methyl-N4-(3- (trifluoromethoxy)benzyl)pyrimido[5,4- d]pyrimidine-2,4,6-triamine | 710.800 |
| 232 | 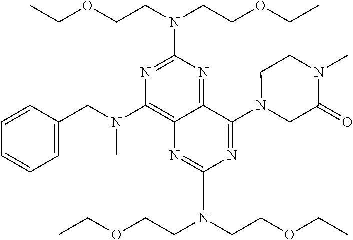 [SMILES: CCOCCN(CCOCC)c1nc(N2CCN(C)C(=O)C2)c2nc(N(CCOCC)CCOCC)nc(N(C)Cc3ccccc3)c2n1] | 4-(8-(benzyl(methyl)amino)-2,6-bis(bis(2- ethoxyethyl)amino)pyrimido[5,4-d]pyrimidin- 4-yl)-1-methylpiperazin-2-one | 681.883 |
| 233 | 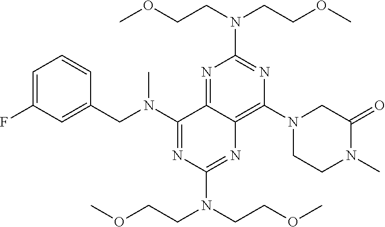 [SMILES: COCCN(CCOC)c1nc(N2CCN(C)C(=O)C2)c2nc(N(CCOC)CCOC)nc(N(C)Cc3cccc(F)c3)c2n1] | 4-(2,6-bis(bis(2-methoxyethyl)amino)-8-((3- fluorobenzyl)(methyl)amino)pyrimido[5,4- d]pyrimidin-4-yl)-1-methylpiperazin-2-one | 643.765 |
| 234 | 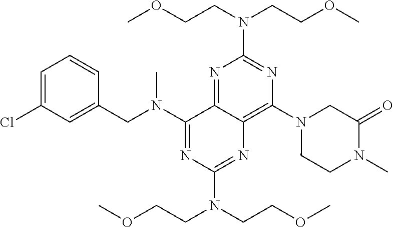 [SMILES: COCCN(CCOC)c1nc(N2CCN(C)C(=O)C2)c2nc(N(CCOC)CCOC)nc(N(C)Cc3cccc(Cl)c3)c2n1] | 4-(2,6-bis(bis(2-methoxyethyl)amino)-8-((3- chlorobenzyl)(methyl)amino)pyrimido[5,4- d]pyrimidin-4-yl)-1-methylpiperazin-2-one | 660.217 |
| 235 | 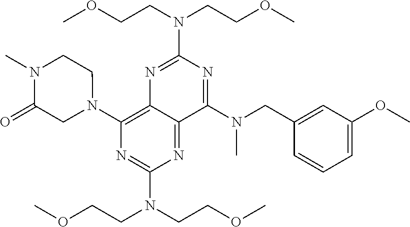 | 4-(2,6-bis(bis(2-methoxyethyl)amino)-8-((3- methoxybenzyl)(methyl)amino)pyrimido[5,4- d]pyrimidin-4-yl)-1-methylpiperazin-2-one | 655.801 |
| 236 | 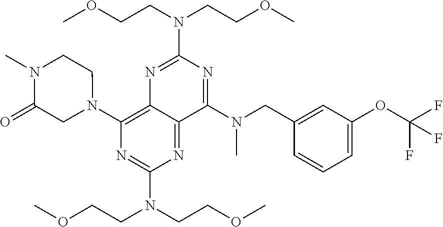 [SMILES: COCCN(CCOC)c1nc(N2CCN(C)C(=O)C2)c2nc(N(CCOC)CCOC)nc(N(C)Cc3cccc(OC(F)(F)F)c3)c2n1] | 4-(2,6-bis(bis(2-methoxyethyl)amino)-8- (methyl(3- (trifluoromethoxy)benzyl)amino)pyrimido[5,4- d]pyrimidin-4-yl)-1-methylpiperazin-2-one | 709.772 |
| 237 | 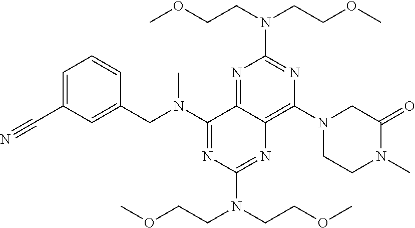 [SMILES: COCCN(CCOC)c1nc(N2CCN(C)C(=O)C2)c2nc(N(CCOC)CCOC)nc(N(C)Cc3cccc(C#N)c3)c2n1] | 3-(((2,6-bis(bis(2-methoxyethyl)amino)-8-(4- methyl-3-oxopiperazin-1-yl)pyrimido[5,4- d]pyrimidin-4- yl)(methyl)amino)methyl)benzonitrile | 650.785 |
| 238 | 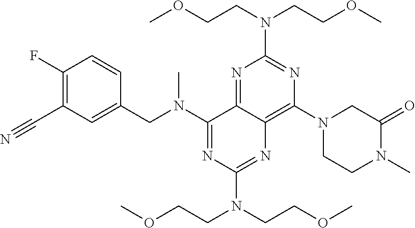 [SMILES: COCCN(CCOC)c1nc(N2CCN(C)C(=O)C2)c2nc(N(CCOC)CCOC)nc(N(C)Cc3ccc(F)c(C#N)c3)c2n1] | 5-(((2,6-bis(bis(2-methoxyethyl)amino)-8-(4- methyl-3-oxopiperazin-1-yl)pyrimido[5,4- d]pyrimidin-4-yl)(methyl)amino)methyl)-2- fluorobenzonitrile | 668.775 |
| 239 | 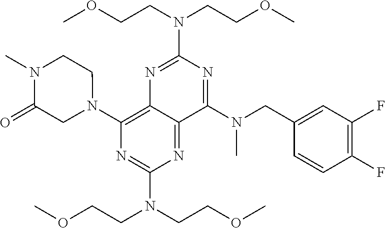 [SMILES: COCCN(CCOC)c1nc(N2CCN(C)C(=O)C2)c2nc(N(CCOC)CCOC)nc(N(C)Cc3ccc(F)c(F)c3)c2n1] | 4-(2,6-bis(bis(2-methoxyethyl)amino)-8-((3,4- difluorobenzyl)(methyl)amino)pyrimido[5,4- d]pyrimidin-4-yl)-1-methylpiperazin-2-one | 661.755 |
| 240 | 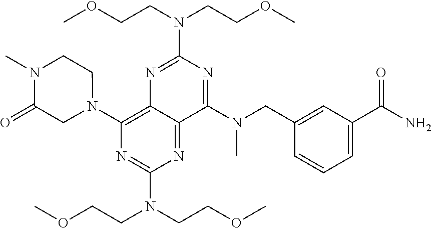 [SMILES: COCCN(CCOC)c1nc(N2CCN(C)C(=O)C2)c2nc(N(CCOC)CCOC)nc(N(C)Cc3cccc(C(N)=O)c3)c2n1] | 3-(((2,6-bis(bis(2-methoxyethyl)amino)-8-(4- methyl-3-oxopiperazin-1-yl)pyrimido[5,4- d]pyrimidin-4- yl)(methyl)amino)methyl)benzamide | 668.800 |
| 241 | 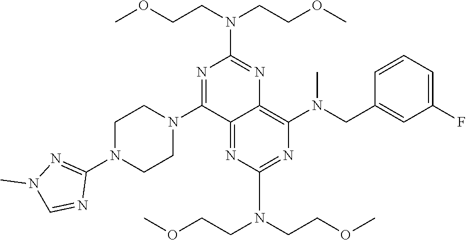 [SMILES: COCCN(CCOC)c1nc(N2CCN(c3ncn(C)n3)CC2)c2nc(N(CCOC)CCOC)nc(N(C)Cc3cccc(F)c3)c2n1] | N4-(3-fluorobenzyl)-N2,N2,N6,N6-tetrakis(2- methoxyethyl)-N4-methyl-8-(4-(1-methyl-1H- 1,2,4-triazol-3-yl)piperazin-1-yl)pyrimido[5,4- d]pyrimidine-2,4,6-triamine | 696.833 |
| 242 | 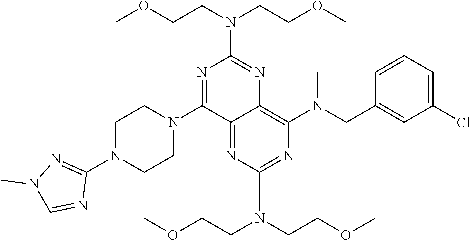 [SMILES: COCCN(CCOC)c1nc(N2CCN(c3ncn(C)n3)CC2)c2nc(N(CCOC)CCOC)nc(N(C)Cc3cccc(Cl)c3)c2n1] | N4-(3-chlorobenzyl)-N2,N2,N6,N6-tetrakis(2- methoxyethyl)-N4-methyl-8-(4-(1-methyl-1H- 1,2,4-triazol-3-yl)piperazin-1-yl)pyrimido[5,4- d]pyrimidine-2,4,6-triamine | 713.285 |
| 243 | 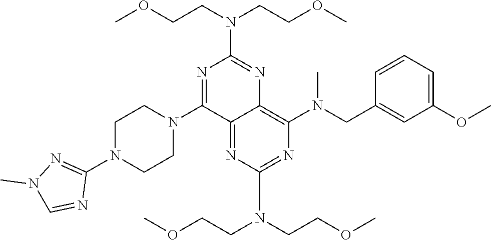 | N4-(3-methoxybenzyl)-N2,N2,N6,N6- tetrakis(2-methoxyethyl)-N4-methyl-8-(4-(1- methyl-1H-1,2,4-triazol-3-yl)piperazin-1- yl)pyrimido[5,4-d]pyrimidine-2,4,6-triamine | 708.869 |
| 244 | 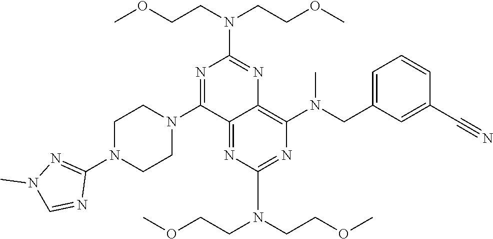 [SMILES: COCCN(CCOC)c1nc(N2CCN(c3ncn(C)n3)CC2)c2nc(N(CCOC)CCOC)nc(N(C)Cc3cccc(C#N)c3)c2n1] | 3-(((2,6-bis(bis(2-methoxyethyl)amino)-8-(4- (1-methyl-1H-1,2,4-triazol-3-yl)piperazin-1- yl)pyrimido[5,4-d]pyrimidin-4- yl)(methyl)amino)methyl)benzonitrile | 703.853 |
| 245 | 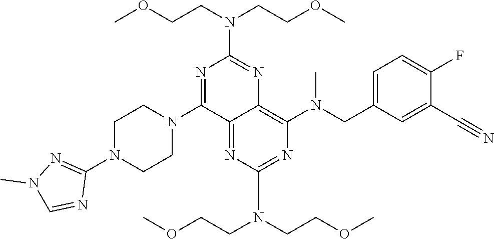 [SMILES: COCCN(CCOC)c1nc(N2CCN(c3ncn(C)n3)CC2)c2nc(N(CCOC)CCOC)nc(N(C)Cc3ccc(F)c(C#N)c3)c2n1] | 5-(((2,6-bis(bis(2-methoxyethyl)amino)-8-(4- (1-methyl-1H-1,2,4-triazol-3-yl)piperazin-1- yl)pyrimido[5,4-d]pyrimidin-4- yl)(methyl)amino)methyl)-2-fluorobenzonitrile | 721.843 |
| 246 | 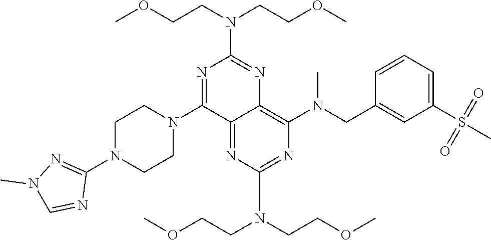 | N2,N2,N6,N6-tetrakis(2-methoxyethyl)-N4- methyl-8-(4-(1-methyl-1H-1,2,4-triazol-3- yl)piperazin-1-yl)-N4-(3- (methylsulfonyl)benzyl)pyrimido[5,4- d]pyrimidine-2,4,6-triamine | 756.928 |
| 247 | 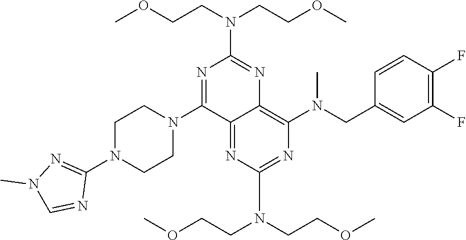 [SMILES: COCCN(CCOC)c1nc(N2CCN(c3ncn(C)n3)CC2)c2nc(N(CCOC)CCOC)nc(N(C)Cc3ccc(F)c(F)c3)c2n1] | N4-(3,4-difluorobenzyl)-N2,N2,N6,N6- tetrakis(2-methoxyethyl)-N4-methyl-8-(4-(1- methyl-1H-1,2,4-triazol-3-yl)piperazin-1- yl)pyrimido[5,4-d]pyrimidine-2,4,6-triamine | 714.823 |
| 248 | 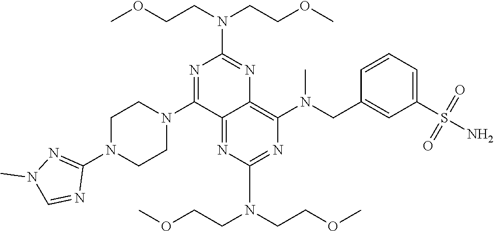 [SMILES: COCCN(CCOC)c1nc(N2CCN(c3ncn(C)n3)CC2)c2nc(N(CCOC)CCOC)nc(N(C)Cc3cccc(S(N)(=O)=O)c3)c2n1] | 3-(((2,6-bis(bis(2-methoxyethyl)amino)-8-(4- (1-methyl-1H-1,2,4-triazol-3-yl)piperazin-1- yl)pyrimido[5,4-d]pyrimidin-4- yl)(methyl)amino)methyl)benzenesulfonamide | 757.916 |
| 249 | 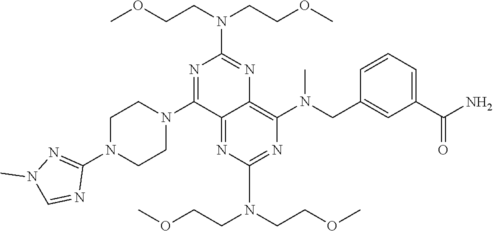 [SMILES: COCCN(CCOC)c1nc(N2CCN(c3ncn(C)n3)CC2)c2nc(N(CCOC)CCOC)nc(N(C)Cc3cccc(C(N)=O)c3)c2n1] | 3-(((2,6-bis(bis(2-methoxyethyl)amino)-8-(4- (1-methyl-1H-1,2,4-triazol-3-yl)piperazin-1- yl)pyrimido[5,4-d]pyrimidin-4- yl)(methyl)amino)methyl)benzamide | 721.868 |
| 250 | 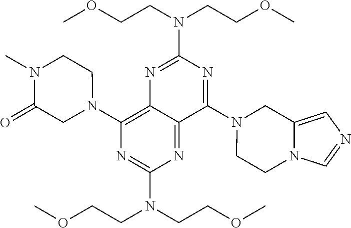 [SMILES: COCCN(CCOC)c1nc(N2CCn3cncc3C2)c2nc(N(CCOC)CCOC)nc(N3CCN(C)C(=O)C3)c2n1] | 4-(2,6-bis(bis(2-methoxyethyl)amino)-8-(5,6- dihydroimidazo[1,5-a]pyrazin-7(8H)- yl)pyrimido[5,4-d]pyrimidin-4-yl)-1- methylpiperazin-2-one | 627.751 |
| 251 | 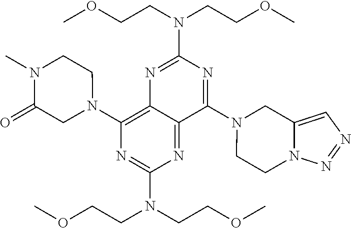 [SMILES: COCCN(CCOC)c1nc(N2CCn3nncc3C2)c2nc(N(CCOC)CCOC)nc(N3CCN(C)C(=O)C3)c2n1] | 4-(2,6-bis(bis(2-methoxyethyl)amino)-8-(6,7- dihydro-[1,2,3]triazolo[1,5-a]pyrazin-5(4H)- yl)pyrimido[5,4-d]pyrimidin-4-yl)-1- methylpiperazin-2-one | 628.739 |
| 252 | 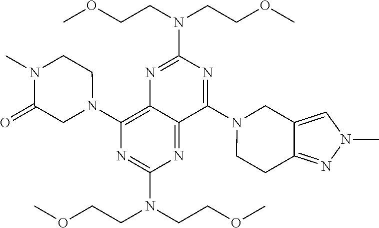 | 4-(2,6-bis(bis(2-methoxyethyl)amino)-8-(2- methyl-2,4,6,7-tetrahydro-5H-pyrazolo[4,3- c]pyridin-5-yl)pyrimido[5,4-d]pyrimidin-4-yl)- 1-methylpiperazin-2-one | 641.778 |
| 253 | 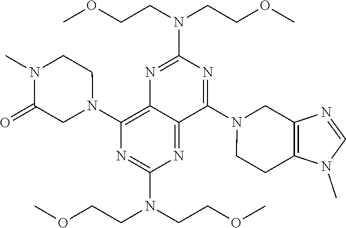 [SMILES: COCCN(CCOC)c1nc(N2CCc3c(ncn3C)C2)c2nc(N(CCOC)CCOC)nc(N3CCN(C)C(=O)C3)c2n1] | 4-(2,6-bis(bis(2-methoxyethyl)amino)-8-(1- methyl-1,4,6,7-tetrahydro-5H-imidazo[4,5- c]pyridin-5-yl)pyrimido[5,4-d]pyrimidin-4-yl)- 1-methylpiperazin-2-one | 641.778 |
| 254 | 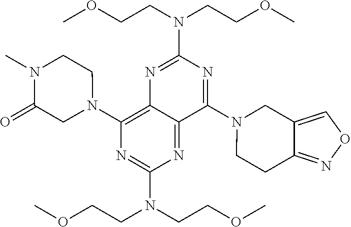 [SMILES: COCCN(CCOC)c1nc(N2CCc3nocc3C2)c2nc(N(CCOC)CCOC)nc(N3CCN(C)C(=O)C3)c2n1] | 4-(2,6-bis(bis(2-methoxyethyl)amino)-8-(6,7- dihydroisoxazolo[4,3-c]pyridin-5(4H)- yl)pyrimido[5,4-d]pyrimidin-4-yl)-1- methylpiperazin-2-one | 628.735 |
| 255 | 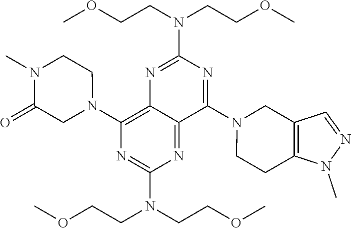 [SMILES: COCCN(CCOC)c1nc(N2CCc3c(cnn3C)C2)c2nc(N(CCOC)CCOC)nc(N3CCN(C)C(=O)C3)c2n1] | 4-(2,6-bis(bis(2-methoxyethyl)amino)-8-(1- methyl-1,4,6,7-tetrahydro-5H-pyrazolo[4,3- c]pyridin-5-yl)pyrimido[5,4-d]pyrimidin-4-yl)- 1-methylpiperazin-2-one | 641.778 |
| 256 | 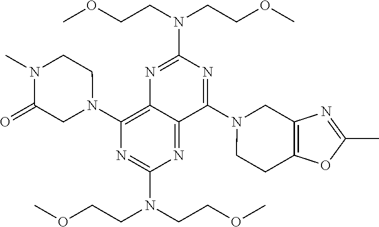 | 4-(2,6-bis(bis(2-methoxyethyl)amino)-8-(2- methyl-6,7-dihydrooxazolo[4,5-c]pyridin- 5(4H)-yl)pyrimido[5,4-d]pyrimidin-4-yl)-1- methylpiperazin-2-one | 642.762 |
| 257 | 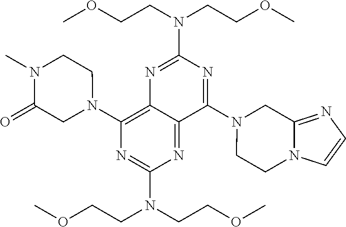 [SMILES: COCCN(CCOC)c1nc(N2CCn3ccnc3C2)c2nc(N(CCOC)CCOC)nc(N3CCN(C)C(=O)C3)c2n1] | 4-(2,6-bis(bis(2-methoxyethyl)amino)-8-(5,6- dihydroimidazo[1,2-a]pyrazin-7(8H)- yl)pyrimido[5,4-d]pyrimidin-4-yl)-1- methylpiperazin-2-one | 627.751 |
| 258 | 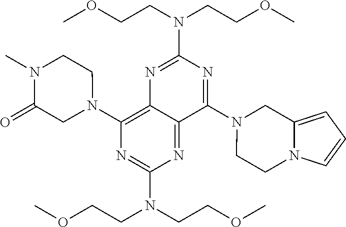 [SMILES: COCCN(CCOC)c1nc(N2CCn3cccc3C2)c2nc(N(CCOC)CCOC)nc(N3CCN(C)C(=O)C3)c2n1] | 4-(2,6-bis(bis(2-methoxyethyl)amino)-8-(3,4- dihydropyrrolo[1,2-a]pyrazin-2(1H)- yl)pyrimido[5,4-d]pyrimidin-4-yl)-1- methylpiperazin-2-one | 626.763 |
| 259 | 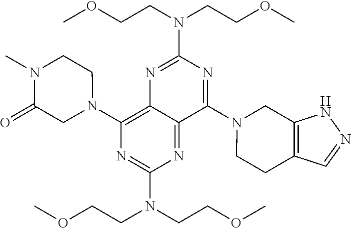 [SMILES: COCCN(CCOC)c1nc(N2CCc3cn[nH]c3C2)c2nc(N(CCOC)CCOC)nc(N3CCN(C)C(=O)C3)c2n1] | 4-(2,6-bis(bis(2-methoxyethyl)amino)-8- (1,4,5,7-tetrahydro-6H-pyrazolo[3,4-c]pyridin- 6-yl)pyrimido[5,4-d]pyrimidin-4-yl)-1- methylpiperazin-2-one | 627.751 |
| 260 | 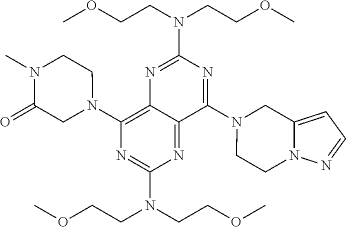 [SMILES: COCCN(CCOC)c1nc(N2CCn3nccc3C2)c2nc(N(CCOC)CCOC)nc(N3CCN(C)C(=O)C3)c2n1] | 4-(2,6-bis(bis(2-methoxyethyl)amino)-8-(6,7- dihydropyrazolo[1,5-a]pyrazin-5(4H)- yl)pyrimido[5,4-d]pyrimidin-4-yl)-1- methylpiperazin-2-one | 627.751 |
| 261 | 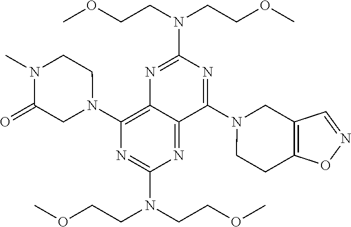 [SMILES: COCCN(CCOC)c1nc(N2CCc3oncc3C2)c2nc(N(CCOC)CCOC)nc(N3CCN(C)C(=O)C3)c2n1] | 4-(2,6-bis(bis(2-methoxyethyl)amino)-8-(6,7- dihydroisoxazolo[4,5-c]pyridin-5(4H)- yl)pyrimido[5,4-d]pyrimidin-4-yl)-1- methylpiperazin-2-one | 628.735 |
| 262 | 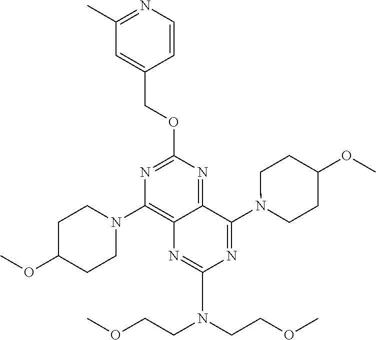 | N,N-bis(2-methoxyethyl)-4,8-bis(4- methoxypiperidin-1-yl)-6-((2-methylpyridin-4- yl)methoxy)pyrimido[5,4-d]pyrimidin-2-amine | 610.76 |
| 263 | 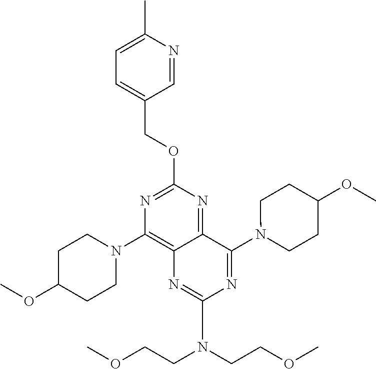 | N,N-bis(2-methoxyethyl)-4,8-bis(4- methoxypiperidin-1-yl)-6-((6-methylpyridin-3- yl)methoxy)pyrimido[5,4-d]pyrimidin-2-amine | 610.76 |
| 264 | 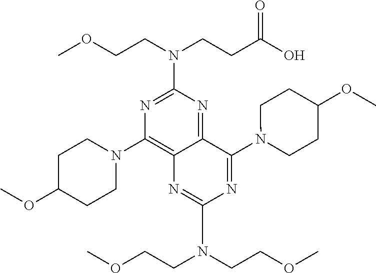 | 3-((6-(bis(2-methoxyethyl)amino)-4,8-bis(4- methoxypiperidin-1-yl)pyrimido[5,4- d]pyrimidin-2-yl)(2- methoxyethyl)amino)propanoic acid | 634.779 |
| 265 | 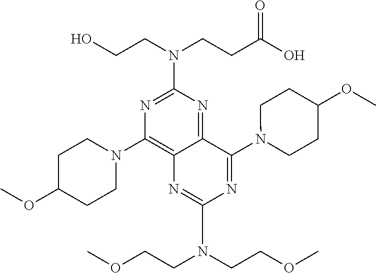 | 3-((6-(bis(2-methoxyethyl)amino)-4,8-bis(4- methoxypiperidin-1-yl)pyrimido[5,4- d]pyrimidin-2-yl)(2- hydroxyethyl)amino)propanoic acid | 620.752 |
| 266 | 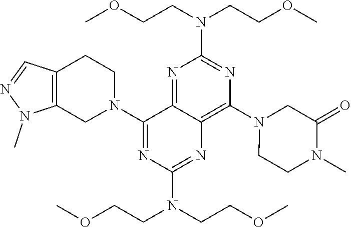 [SMILES: COCCN(CCOC)c1nc(N2CCc3cnn(C)c3C2)c2nc(N(CCOC)CCOC)nc(N3CCN(C)C(=O)C3)c2n1] | 4-(2,6-bis(bis(2-methoxyethyl)amino)-8-(1- methyl-1,4,5,7-tetrahydro-6H-pyrazolo[3,4- c]pyridin-6-yl)pyrimido[5,4-d]pyrimidin-4-yl)- 1-methylpiperazin-2-one | 641.778 |
| 267 | 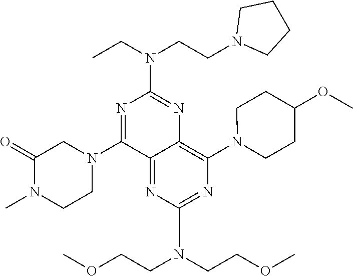 | 4-(6-(bis(2-methoxyethyl)amino)-2-(ethyl(2- (pyrrolidin-1-yl)ethyl)amino)-8-(4- methoxypiperidin-1-yl)pyrimido[5,4- d]pyrimidin-4-yl)-1-methylpiperazin-2-one | 628.823 |
| 268 | 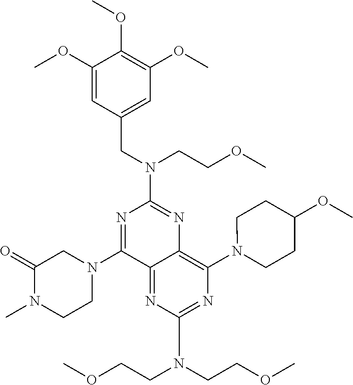 | 4-(6-(bis(2-methoxyethyl)amino)-2-((2- methoxyethyl)(3,4,5- trimethoxybenzyl)amino)-8-(4- methoxypiperidin-1-yl)pyrimido[5,4- d]pyrimidin-4-yl)-1-methylpiperazin-2-one | 741.891 |
| 269 | 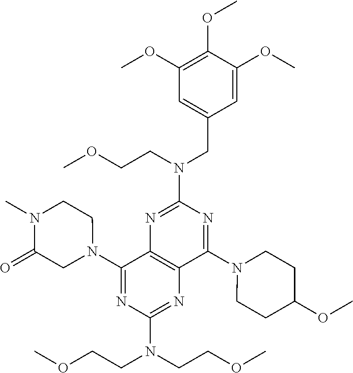 | 4-(2-(bis(2-methoxyethyl)amino)-6-((2- methoxyethyl)(3,4,5- trimethoxybenzyl)amino)-8-(4- methoxypiperidin-1-yl)pyrimido[5,4- d]pyrimidin-4-yl)-1-methylpiperazin-2-one | 741.891 |
| 270 | 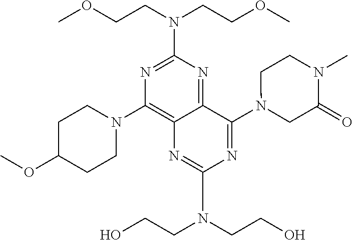 [SMILES: COCCN(CCOC)c1nc(N2CCC(OC)CC2)c2nc(N(CCO)CCO)nc(N3CCN(C)C(=O)C3)c2n1] | 4-(2-(bis(2-hydroxyethyl)amino)-6-(bis(2- methoxyethyl)amino)-8-(4-methoxypiperidin- 1-yl)pyrimido[5,4-d]pyrimidin-4-yl)-1- methylpiperazin-2-one | 591.714 |
| 271 | 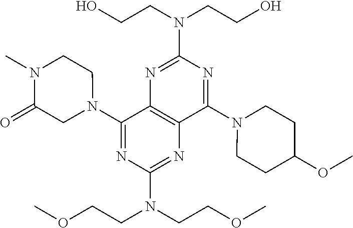 | 4-(6-(bis(2-hydroxyethyl)amino)-2-(bis(2- methoxyethyl)amino)-8-(4-methoxypiperidin- 1-yl)pyrimido[5,4-d]pyrimidin-4-yl)-1- methylpiperazin-2-one | 591.714 |
| 272 | 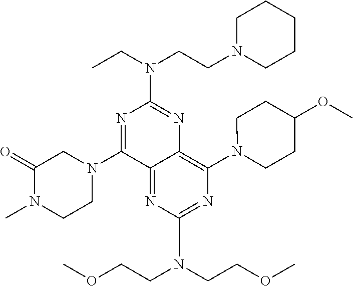 | 4-(6-(bis(2-methoxyethyl)amino)-2-(ethyl(2- (piperidin-1-yl)ethyl)amino)-8-(4- methoxypiperidin-1-yl)pyrimido[5,4- d]pyrimidin-4-yl)-1-methylpiperazin-2-one | 642.85 |
| 273 | 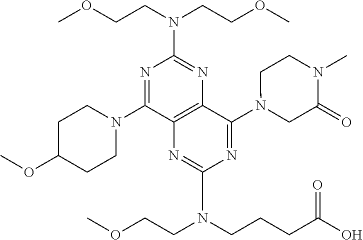 [SMILES: COCCN(CCCC(=O)O)c1nc(N2CCN(C)C(=O)C2)c2nc(N(CCOC)CCOC)nc(N3CCC(OC)CC3)c2n1] | 4-((6-(bis(2-methoxyethyl)amino)-8-(4- methoxypiperidin-1-yl)-4-(4-methyl-3- oxopiperazin-1-yl)pyrimido[5,4-d]pyrimidin-2- yl)(2-methoxyethyl)amino)butanoic acid | 647.778 |
| 274 | 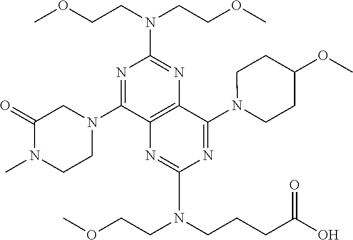 | 4-((6-(bis(2-methoxyethyl)amino)-4-(4- methoxypiperidin-1-yl)-8-(4-methyl-3- oxopiperazin-1-yl)pyrimido[5,4-d]pyrimidin-2- yl)(2-methoxyethyl)amino)butanoic acid | 647.778 |
| 275 | 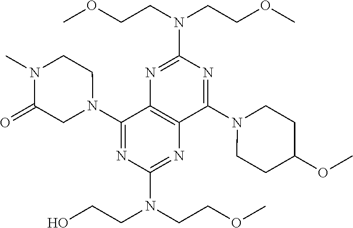 | 4-(6-(bis(2-methoxyethyl)amino)-2-((2- hydroxyethyl)(2-methoxyethyl)amino)-8-(4- methoxypiperidin-1-yl)pyrimido[5,4- d]pyrimidin-4-yl)-1-methylpiperazin-2-one | 605.741 |
| 276 | 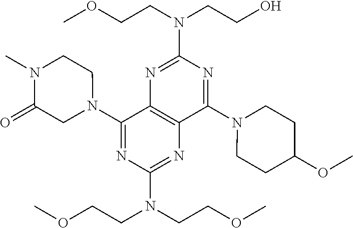 | 4-(2-(bis(2-methoxyethyl)amino)-6-((2- hydroxyethyl)(2-methoxyethyl)amino)-8-(4- methoxypiperidin-1-yl)pyrimido[5,4- d]pyrimidin-4-yl)-1-methylpiperazin-2-one | 605.741 |
| 277 | 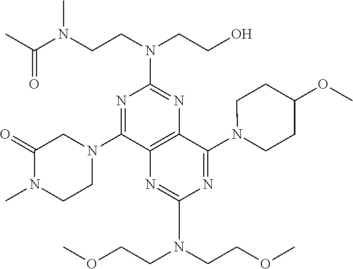 | N-(2-((6-(bis(2-methoxyethyl)amino)-8-(4- methoxypiperidin-1-yl)-4-(4-methyl-3- oxopiperazin-1-yl)pyrimido[5,4-d]pyrimidin-2- yl)(2-hydroxyethyl)amino)ethyl)-N- methylacetamide | 646.794 |
| 278 | 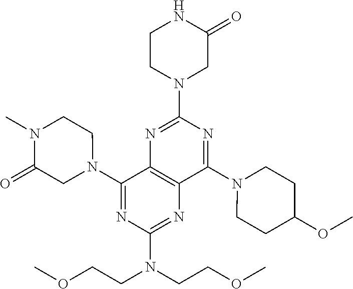 | 4-(2-(bis(2-methoxyethyl)amino)-8-(4- methoxypiperidin-1-yl)-6-(3-oxopiperazin-1- yl)pyrimido[5,4-d]pyrimidin-4-yl)-1- methylpiperazin-2-one | 586.698 |
| 279 | 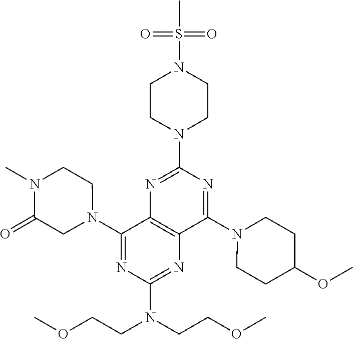 | 4-(2-(bis(2-methoxyethyl)amino)-8-(4- methoxypiperidin-1-yl)-6-(4- (methylsulfonyl)piperazin-1-yl)pyrimido[5,4- d]pyrimidin-4-yl)-1-methylpiperazin-2-one | 650.8 |
| 280 | 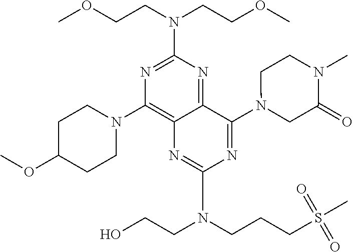 | 4-(6-(bis(2-methoxyethyl)amino)-2-((2- hydroxyethyl)(3- (methylsulfonyl)propyl)amino)-8-(4- methoxypiperidin-1-yl)pyrimido[5,4- d]pyrimidin-4-yl)-1-methylpiperazin-2-one | 667.827 |
| 281 | 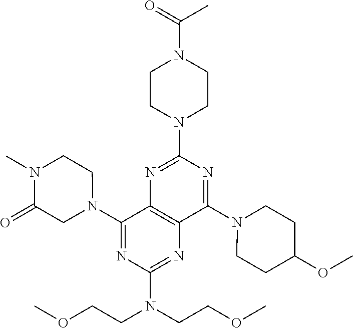 | 4-(6-(4-acetylpiperazin-1-yl)-2-(bis(2- methoxyethyl)amino)-8-(4-methoxypiperidin- 1-yl)pyrimido[5,4-d]pyrimidin-4-yl)-1- methylpiperazin-2-one | 614.752 |
| 282 | 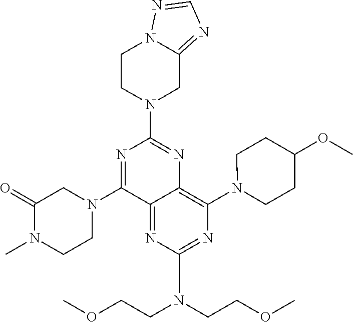 | 4-(6-(bis(2-methoxyethyl)amino)-2-(5,6- dihydro-[1,2,4]triazolo[1,5-a]pyrazin-7(8H)- yl)-8-(4-methoxypiperidin-1-yl)pyrimido[5,4- d]pyrimidin-4-yl)-1-methylpiperazin-2-one | 610.724 |
| 283 | 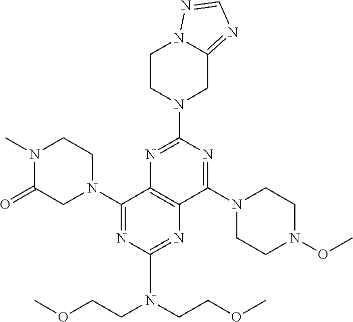 | 4-(2-(bis(2-methoxyethyl)amino)-6-(5,6- dihydro-[1,2,4]triazolo[1,5-a]pyrazin-7(8H)- yl)-8-(4-methoxypiperidin-1-yl)pyrimido[5,4- d]pyrimidin-4-yl)-1-methylpiperazin-2-one | 610.724 |
| 284 | 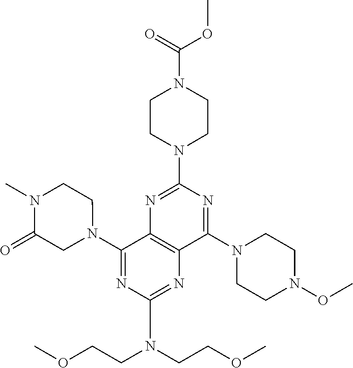 | methyl 4-(6-(bis(2-methoxyethyl)amino)-4-(4- methoxypiperidin-1-yl)-8-(4-methyl-3- oxopiperazin-1-yl)pyrimido[5,4-d]pyrimidin-2- yl)piperazine-1-carboxylate | 630.751 |
| 285 | 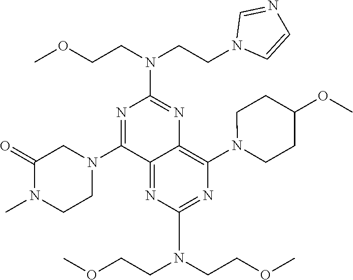 | 4-(2-((2-(1H-imidazol-1-yl)ethyl)(2- methoxyethyl)amino)-6-(bis(2- methoxyethyl)amino)-8-(4-methoxypiperidin- 1-yl)pyrimido[5,4-d]pyrimidin-4-yl)-1- methylpiperazin-2-one | 655.805 |
| 286 | 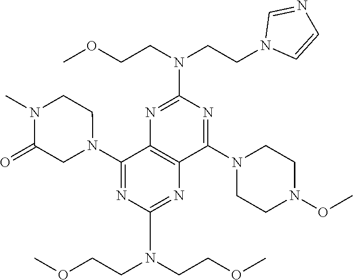 | 4-(6-((2-(1H-imidazol-1-yl)ethyl)(2- methoxyethyl)amino)-2-(bis(2- methoxyethyl)amino)-8-(4-methoxypiperidin- 1-yl)pyrimido[5,4-d]pyrimidin-4-yl)-1- methylpiperazin-2-one | 655.805 |
| 287 | 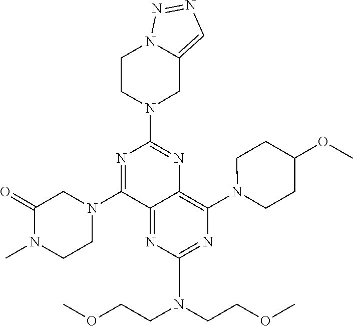 | 4-(6-(bis(2-methoxyethyl)amino)-2-(6,7- dihydro-[1,2,3]triazolo[1,5-a]pyrazin-5(4H)- yl)-8-(4-methoxypiperidin-1-yl)pyrimido[5,4- d]pyrimidin-4-yl)-1-methylpiperazin-2-one | 610.724 |
| 288 | 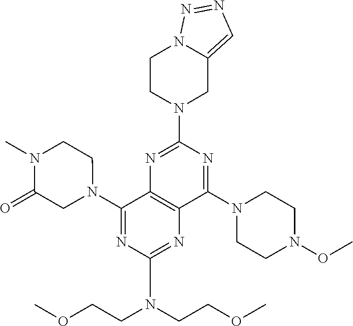 | 4-(2-(bis(2-methoxyethyl)amino)-6-(6,7- dihydro-[1,2,3]triazolo[1,5-a]pyrazin-5(4H)- yl)-8-(4-methoxypiperidin-1-yl)pyrimido[5,4- d]pyrimidin-4-yl)-1-methylpiperazin-2-one | 610.724 |
| 289 | 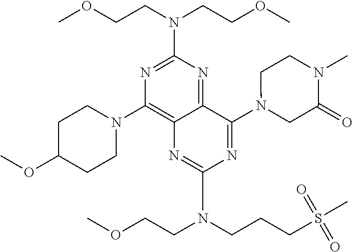 | 4-(6-(bis(2-methoxyethyl)amino)-2-((2- methoxyethyl)(3- (methylsulfonyl)propyl)amino)-8-(4- methoxypiperidin-1-yl)pyrimido[5,4- d]pyrimidin-4-yl)-1-methylpiperazin-2-one | 681.854 |
| 290 | 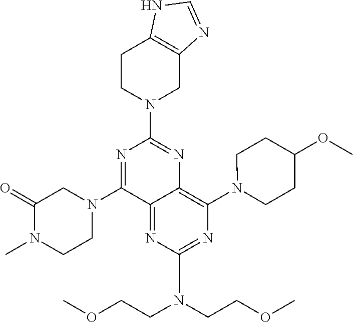 | 4-(6-(bis(2-methoxyethyl)amino)-8-(4- methoxypiperidin-1-yl)-2-(1,4,6,7-tetrahydro- 5H-imidazo[4,5-c]pyridin-5-yl)pyrimido[5,4- d]pyrimidin-4-yl)-1-methylpiperazin-2-one | 609.736 |
| 291 | 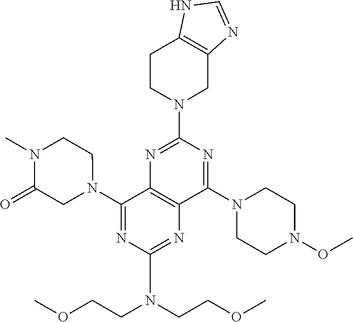 | 4-(2-(bis(2-methoxyethyl)amino)-8-(4- methoxypiperidin-1-yl)-6-(1,4,6,7-tetrahydro- 5H-imidazo[4,5-c]pyridin-5-yl)pyrimido[5,4- d]pyrimidin-4-yl)-1-methylpiperazin-2-one | 609.736 |
| 292 | 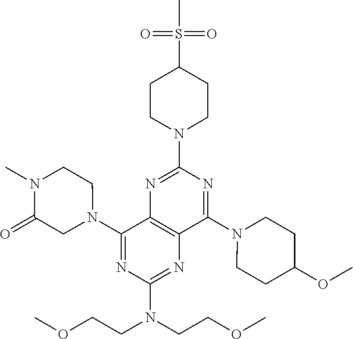 | 4-(2-(bis(2-methoxyethyl)amino)-8-(4- methoxypiperidin-1-yl)-6-(4- (methyisulfonyl)piperidin-1-yl)pyrimido[5,4- d]pyrimidin-4-yl)-1-methylpiperazin-2-one | 649.812 |
| 293 | 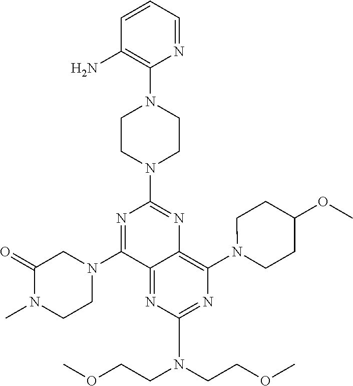 | 4-(2-(4-(3-aminopyridin-2-yl)piperazin-1-yl)- 6-(bis(2-methoxyethyl)amino)-8-(4- methoxypiperidin-1-yl)pyrimido[5,4- d]pyrimidin-4-yl)-1-methylpiperazin-2-one | 664.816 |
| 294 | 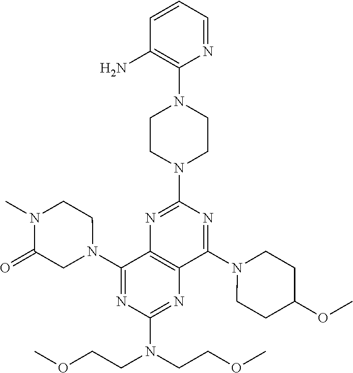 | 4-(6-(4-(3-aminopyridin-2-yl)piperazin-1-yl)- 2-(bis(2-methoxyethyl)amino)-8-(4- methoxypiperidin-1-yl)pyrimido[5,4- d]pyrimidin-4-yl)-1-methylpiperazin-2-one | 664.816 |
| 295 | 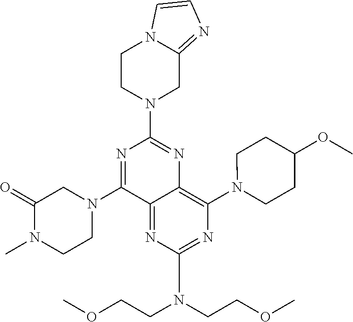 | 4-(6-(bis(2-methoxyethyl)amino)-2-(5,6- dihydroimidazo[1,2-a]pyrazin-7(8H)-yl)-8-(4- methoxypiperidin-1-yl)pyrimido[5,4- d]pyrimidin-4-yl)-1-methylpiperazin-2-one | 609.736 |
| 296 | 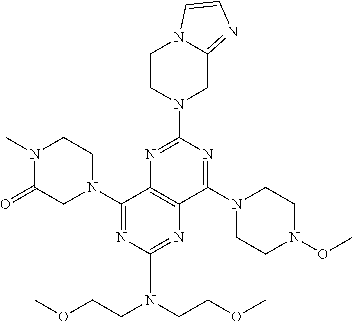 | 4-(2-(bis(2-methoxyethyl)amino)-6-(5,6- dihydroimidazo[1,2-a]pyrazin-7(8H)-yl)-8-(4- methoxypiperidin-1-yl)pyrimido[5,4- d]pyrimidin-4-yl)-1-methylpiperazin-2-one | 609.736 |
| 297 | 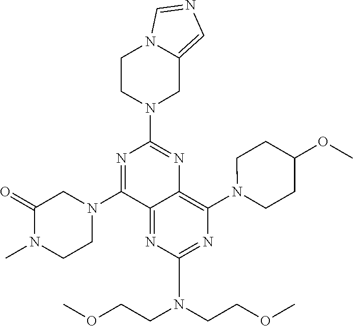 | 4-(6-(bis(2-methoxyethyl)amino)-2-(5,6- dihydroimidazo[1,5-a]pyrazin-7(8H)-yl)-8-(4- methoxypiperidin-1-yl)pyrimido[5,4- d]pyrimidin-4-yl)-1-methylpiperazin-2-one | 609.736 |
| 298 | 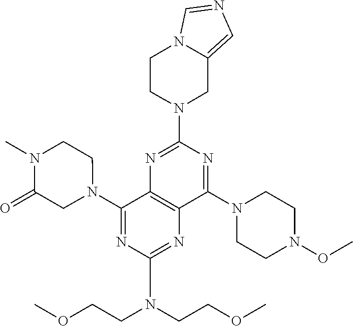 | 4-(2-(bis(2-methoxyethyl)amino)-6-(5,6- dihydroimidazo[1,5-a]pyrazin-7(8H)-yl)-8-(4- methoxypiperidin-1-yl)pyrimido[5,4- d]pyrimidin-4-yl)-1-methylpiperazin-2-one | 609.736 |
| 299 | 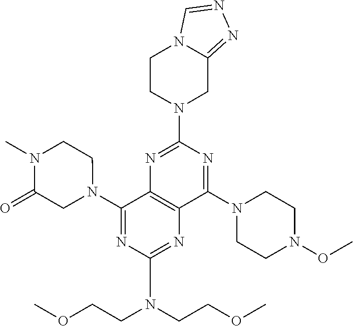 | 4-(2-(bis(2-methoxyethyl)amino)-6-(5,6- dihydro-[1,2,4]triazolo[4,3-a]pyrazin-7(8H)- yl)-8-(4-methoxypiperidin-1-yl)pyrimido[5,4- d]pyrimidin-4-yl)-1-methylpiperazin-2-one | 610.724 |
| 300 | 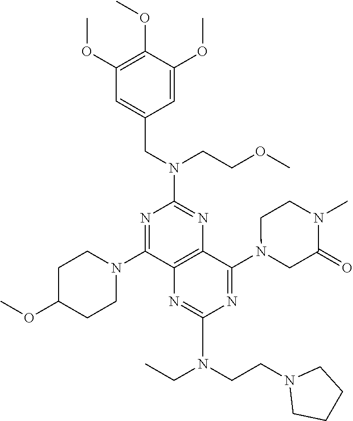 [SMILES: CCN(CCN1CCCC1)c1nc(N2CCN(C)C(=O)C2)c2nc(N(CCOC)Cc3cc(OC)c(OC)c(OC)c3)nc(N3CCC(OC)CC3)c2n1] | 4-(2-(ethyl(2-(pyrrolidin-1-yl)ethyl)amino)-6- ((2-methoxyethyl)(3,4,5- trimethoxybenzyl)amino)-8-(4- methoxypiperidin-1-yl)pyrimido[5,4- d]pyrimidin-4-yl)-1-methylpiperazin-2-one | 750.946 |
| 301 | 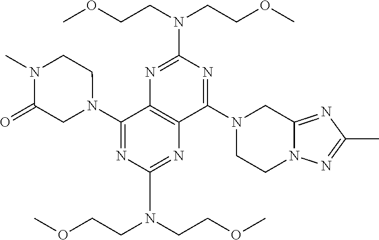 | 4-(2,6-bis(bis(2-methoxyethyl)amino)-8-(2- methyl-5,6-dihydro-[1,2,4]triazolo[1,5- a]pyrazin-7(8H)-yl)pyrimido[5,4-d]pyrimidin- 4-yl)-1-methylpiperazin-2-one | 642.766 |
| 302 | 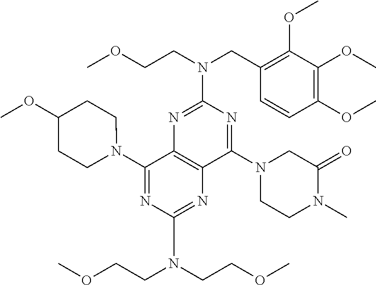 | 4-(6-(bis(2-methoxyethyl)amino)-2-((2- methoxyethyl)(2,3,4- trimethoxybenzyl)amino)-8-(4- methoxypiperidin-1-yl)pyrimido[5,4- d]pyrimidin-4-yl)-1-methylpiperazin-2-one | 741.891 |
| 303 | 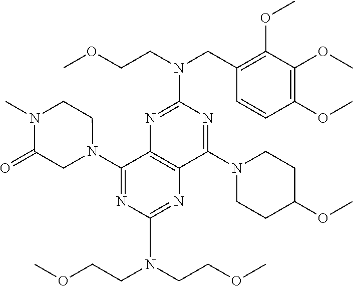 | 4-(2-(bis(2-methoxyethyl)amino)-6-((2- methoxyethyl)(2,3,4- trimethoxybenzyl)amino)-8-(4- methoxypiperidin-1-yl)pyrimido[5,4- d]pyrimidin-4-yl)-1-methylpiperazin-2-one | 741.891 |
| 304 | 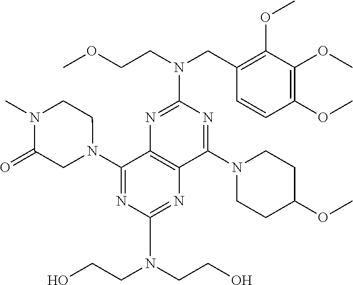 | 4-(2-(bis(2-hydroxyethyl)amino)-6-((2- methoxyethyl)(2,3,4- trimethoxybenzyl)amino)-8-(4- methoxypiperidin-1-yl)pyrimido[5,4- d]pyrimidin-4-yl)-1-methylpiperazin-2-one | 713.837 |
| 305 | 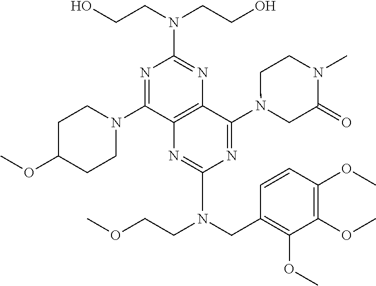 | 4-(6-(bis(2-hydroxyethyl)amino)-2-((2- methoxyethyl)(2,3,4- trimethoxybenzyl)amino)-8-(4- methoxypiperidin-1-yl)pyrimido[5,4- d]pyrimidin-4-yl)-1-methylpiperazin-2-one | 713.837 |
| 306 | 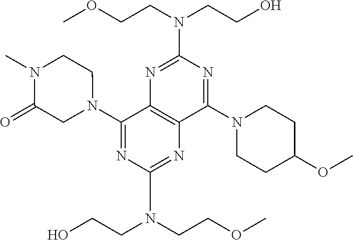 | 4-(2,6-bis((2-hydroxyethyl)(2- methoxyethyl)amino)-8-(4-methoxypiperidin- 1-yl)pyrimido[5,4-d]pyrimidin-4-yl)-1- methylpiperazin-2-one | 591.714 |
| 307 | 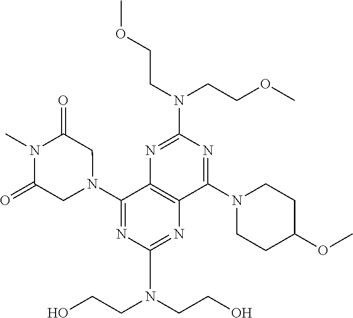 | 4-(2-(bis(2-hydroxyethyl)amino)-6-(bis(2- methoxyethyl)amino)-8-(4-methoxypiperidin- 1-yl)pyrimido[5,4-d]pyrimidin-4-yl)-1- methylpiperazine-2,6-dione | 605.697 |
| 308 | 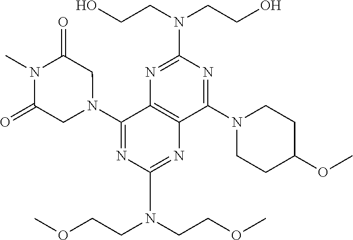 | 4-(6-(bis(2-hydroxyethyl)amino)-2-(bis(2- methoxyethyl)amino)-8-(4-methoxypiperidin- 1-yl)pyrimido[5,4-d]pyrimidin-4-yl)-1- methylpiperazine-2,6-dione | 605.697 |
| 309 | 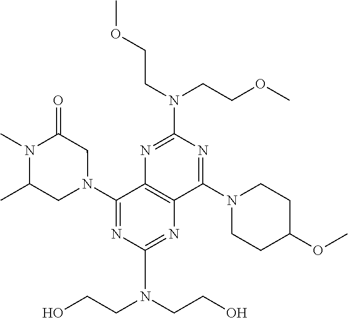 | 4-(2-(bis(2-hydroxyethyl)amino)-6-(bis(2- methoxyethyl)amino)-8-(4-methoxypiperidin- 1-yl)pyrimido[5,4-d]pyrimidin-4-yl)-1,6- dimethylpiperazin-2-one | 605.741 |
| 310 | 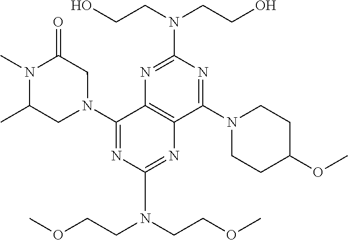 | 4-(6-(bis(2-hydroxyethyl)amino)-2-(bis(2- methoxyethyl)amino)-8-(4-methoxypiperidin- 1-yl)pyrimido[5,4-d]pyrimidin-4-yl)-1,6- dimethylpiperazin-2-one | 605.741 |
| 311 | 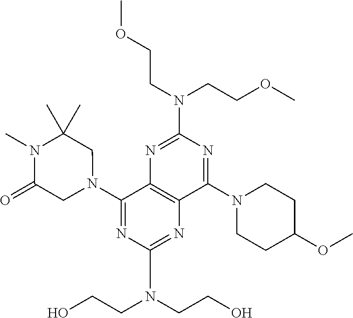 | 4-(2-(bis(2-hydroxyethyl)amino)-6-(bis(2- methoxyethyl)amino)-8-(4-methoxypiperidin- 1-yl)pyrimido[5,4-d]pyrimidin-4-yl)-1,6,6- trimethylpiperazin-2-one | 619.768 |
| 312 | 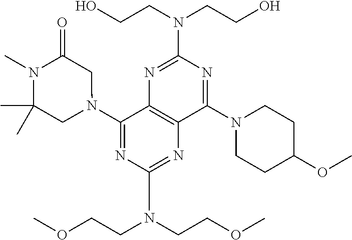 | 4-(6-(bis(2-hydroxyethyl)amino)-2-(bis(2- methoxyethyl)amino)-8-(4-methoxypiperidin- 1-yl)pyrimido[5,4-d]pyrimidin-4-yl)-1,6,6- trimethylpiperazin-2-one | 619.768 |
| 313 | 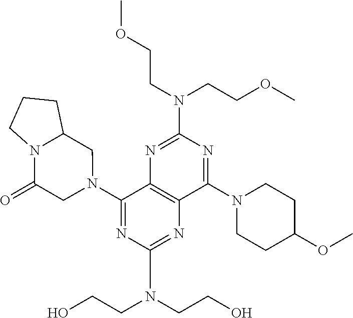 [SMILES: COCCN(CCOC)c1nc(N2CCC(OC)CC2)c2nc(N(CCO)CCO)nc(N3CC(=O)N4CCCC4C3)c2n1] | 2-(2-(bis(2-hydroxyethyl)amino)-6-(bis(2- methoxyethyl)amino)-8-(4-methoxypiperidin- 1-yl)pyrimido[5,4-d]pyrimidin-4- yl)hexahydropyrrolo[1,2-a]pyrazin-4(1H)-one | 617.752 |
| 314 | 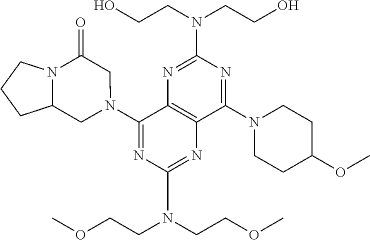 [SMILES: COCCN(CCOC)c1nc(N2CC(=O)N3CCCC3C2)c2nc(N(CCO)CCO)nc(N3CCC(OC)CC3)c2n1] | 2-(6-(bis(2-hydroxyethyl)amino)-2-(bis(2- methoxyethyl)amino)-8-(4-methoxypiperidin- 1-yl)pyrimido[5,4-d]pyrimidin-4- yl)hexahydropyrrolo[1,2-a]pyrazin-4(1H)-one | 617.752 |
| 315 | 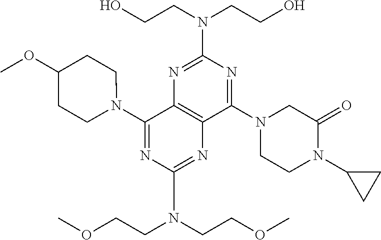 [SMILES: COCCN(CCOC)c1nc(N2CCC(OC)CC2)c2nc(N(CCO)CCO)nc(N3CCN(C4CC4)C(=O)C3)c2n1] | 4-(2-(bis(2-hydroxyethyl)amino)-6-(bis(2- methoxyethyl)amino)-8-(4-methoxypiperidin- 1-yl)pyrimido[5,4-d]pyrimidin-4-yl)-1- cyclopropylpiperazin-2-one | 617.752 |
| 316 | 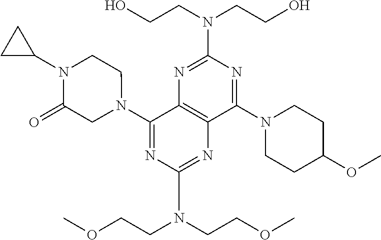 [SMILES: COCCN(CCOC)c1nc(N2CCN(C3CC3)C(=O)C2)c2nc(N(CCO)CCO)nc(N3CCC(OC)CC3)c2n1] | 4-(6-(bis(2-hydroxyethyl)amino)-2-(bis(2- methoxyethyl)amino)-8-(4-methoxypiperidin- 1-yl)pyrimido[5,4-d]pyrimidin-4-yl)-1- cyclopropylpiperazin-2-one | 617.752 |
| 318 | 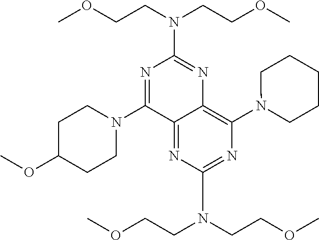 | N2,N2,N6,N6-tetrakis(2-methoxyethyl)-4-(4- methoxypiperidin-1-yl)-8-(piperidin-1- yl)pyrimido[5,4-d]pyrimidine-2,6-diamine | 590.77 |
| 319 | 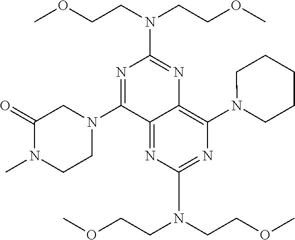 | 4-(2,6-bis(bis(2-methoxyethyl)amino)-8- (piperidin-1-yl)pyrimido[5,4-d]pyrimidin-4- yl)-1-methylpiperazin-2-one | 589.742 |
| 320 | 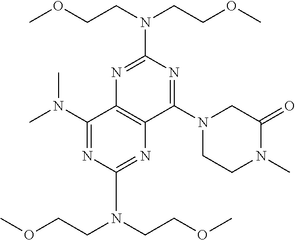 [SMILES: COCCN(CCOC)c1nc(N2CCN(C)C(=O)C2)c2nc(N(CCOC)CCOC)nc(N(C)C)c2n1] | 4-(2,6-bis(bis(2-methoxyethyl)amino)-8- (dimethylamino)pyrimido[5,4-d]pyrimidin-4- yl)-1-methylpiperazin-2-one | 549.677 |
| 321 | 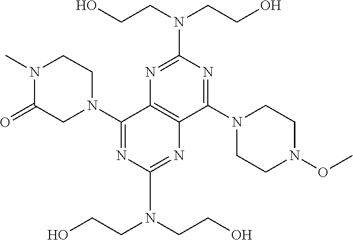 [SMILES: CON1CCN(c2nc(N(CCO)CCO)nc3c(N4CCN(C)C(=O)C4)nc(N(CCO)CCO)nc23)CC1] | 4-(2,6-bis(bis(2-hydroxyethyl)amino)-8-(4- methoxypiperidin-1-yl)pyrimido[5,4- d]pyrimidin-4-yl)-1-methylpiperazin-2-one | 563.66 |
| 322 | 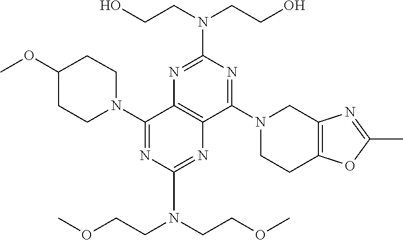 | 2,2′-((6-(bis(2-methoxyethyl)amino)-8-(4- methoxypiperidin-1-yl)-4-(2-methyl-6,7- dihydrooxazolo[4,5-c]pyridin-5(4H)- yl)pyrimido[5,4-d]pyrimidin-2- yl)azanediyl)bis(ethan-1-ol) | 615.736 |
| 323 | 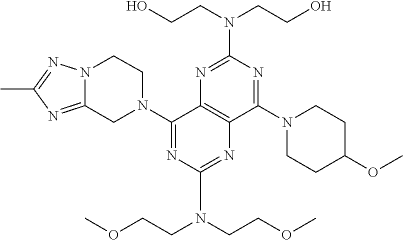 | 2,2′-((6-(bis(2-methoxyethyl)amino)-4-(4- methoxypiperidin-1-yl)-8-(2-methyl-5,6- dihydro-[1,2,4]triazolo[1,5-a]pyrazin-7(8H)- yl)pyrimido[5,4-d]pyrimidin-2- yl)azanediyl)bis(ethan-1-ol) | 615.74 |
| 324 | 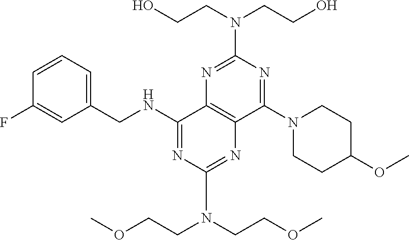 [SMILES: COCCN(CCOC)c1nc(NCc2cccc(F)c2)c2nc(N(CCO)CCO)nc(N3CCC(OC)CC3)c2n1] | 2,2′-((6-(bis(2-methoxyethyl)amino)-8-((3- lluorobenzyl)amino)-4-(4-methoxypiperidin-1- yl)pyrimido[5,4-d]pyrimidin-2- yl)azanediyl)bis(ethan-1-ol) | 602.7124 |
| 325 | 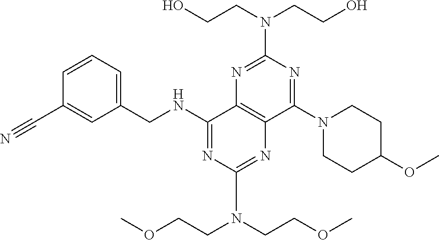 [SMILES: COCCN(CCOC)c1nc(NCc2cccc(C#N)c2)c2nc(N(CCO)CCO)nc(N3CCC(OC)CC3)c2n1] | 3-(((6-(bis(2-hydroxyethyl)amino)-2-(bis(2- methoxyethyl)amino)-8-(4-methoxypiperidin- 1-yl)pyrimido[5,4-d]pyrimidin-4- yl)amino)methyl)benzonitrile | 609.732 |
| 326 | 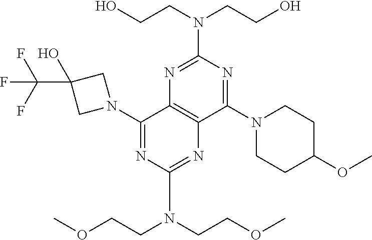 [SMILES: COCCN(CCOC)c1nc(N2CC(O)(C(F)(F)F)C2)c2nc(N(CCO)CCO)nc(N3CCC(OC)CC3)c2n1] | 2,2′-((6-(bis(2-methoxyethyl)amino)-8-(3- hydroxy-3-(trifluoromethyl)azetidin-1-yl)-4-(4- methoxypiperidin-1-yl)pyrimido[5,4- d]pyrimidin-2-yl)azanediyl)bis(ethan-1-ol) | 618.6592 |
| 327 | 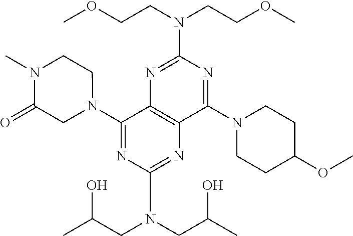 | 4-(2-(bis(2-hydroxypropyl)amino)-6-(bis(2- methoxyethyl)amino)-8-(4-methoxypiperidin- 1-yl)pyrimido[5,4-d]pyrimidin-4-yl)-1- methylpiperazin-2-one | 619.768 |
| 328 | 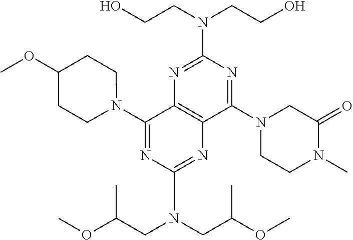 [SMILES: COC(C)CN(CC(C)OC)c1nc(N2CCC(OC)CC2)c2nc(N(CCO)CCO)nc(N3CCN(C)C(=O)C3)c2n1] | 4-(2-(bis(2-hydroxyethyl)amino)-6-(bis(2- methoxypropyl)amino)-8-(4- methoxypiperidin-1-yl)pyrimido[5,4- d]pyrimidin-4-yl)-1-methylpiperazin-2-one | 619.768 |
| 329 | 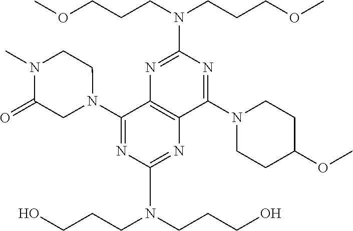 | 4-(2-(bis(3-hydroxypropyl)amino)-6-(bis(3- methoxypropyl)amino)-8-(4- methoxypiperidin-1-yl)pyrimido[5,4- d]pyrimidin-4-yl)-1-methylpiperazin-2-one | 647.822 |
| 330 | 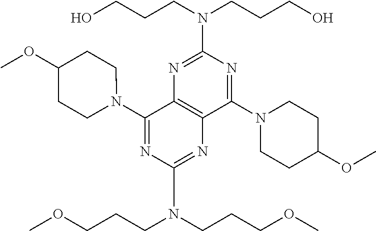 | 3,3′-((6-(bis(3-methoxypropyl)amino)-4,8- bis(4-methoxypiperidin-1-yl)pyrimido[5,4- d]pyrimidin-2-yl)azanediyl)bis(propan-1-ol) | 648.85 |
| 331 | 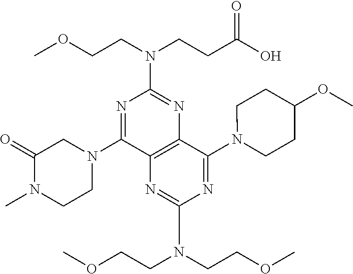 | 3-((6-(bis(2-methoxyethyl)amino)-8-(4- methoxypiperidin-1-yl)-4-(4-methyl-3- oxopiperazin-1-yl)pyrimido[5,4-d]pyrimidin-2- yl)(2-methoxyethyl)amino)propanoic acid | 633.751 |
| 332 | 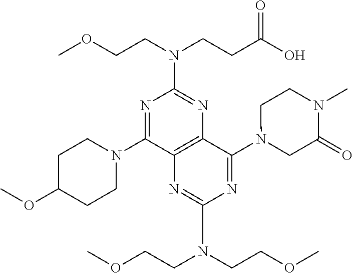 [SMILES: COCCN(CCOC)c1nc(N2CCN(C)C(=O)C2)c2nc(N(CCOC)CCC(=O)O)nc(N3CCC(OC)CC3)c2n1] | 3-((6-(bis(2-methoxyethyl)amino)-4-(4- methoxypiperidin-1-yl)-8-(4-methyl-3- oxopiperazin-1-yl)pyrimido[5,4-d]pyrimidin-2- yl)(2-methoxyethyl)amino)propanoic acid | 633.751 |
| 333 | 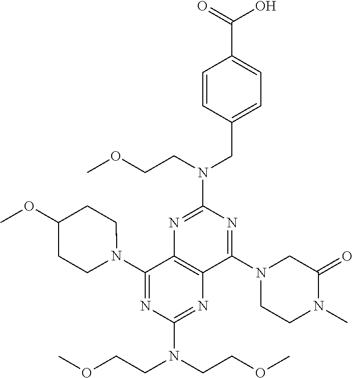 [SMILES: COCCN(Cc1ccc(C(=O)O)cc1)c1nc(N2CCN(C)C(=O)C2)c2nc(N(CCOC)CCOC)nc(N3CCC(OC)CC3)c2n1] | 4-(((6-(bis(2-methoxyethyl)amino)-8-(4- methoxypiperidin-1-yl)-4-(4-methyl-3- oxopiperazin-1-yl)pyrimido[5,4-d]pyrimidin-2- yl)(2-methoxyethyl)amino)methyl)benzoic acid | 695.822 |
| 334 | 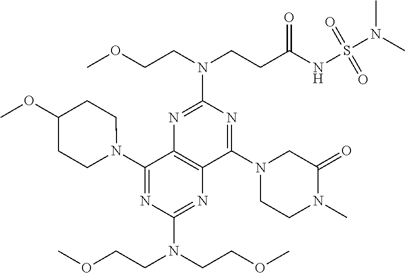 | 3-((6-(bis(2-methoxyethyl)amino)-8-(4- methoxypiperidin-1-yl)-4-(4-methyl-3- oxopiperazin-1-yl)pyrimido[5,4-d]pyrimidin-2- yl)(2-methoxyethyl)amino)-N-(N,N- dimethylsulfamoyl)propanamide | 739.894 |
| 335 | 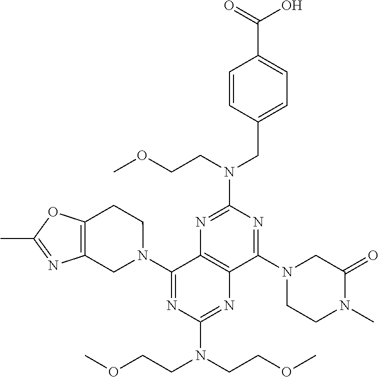 [SMILES: COCCN(CCOC)c1nc(N2CCc3oc(C)nc3C2)c2nc(N(CCOC)Cc3ccc(C(=O)O)cc3)nc(N3CCN(C)C(=O)C3)c2n1] | 4-(((6-(bis(2-methoxyethyl)amino)-4-(4- methyl-3-oxopiperazin-1-yl)-8-(2-methyl-6,7- dihydrooxazolo[4,5-c]pyridin-5(4H)- yl)pyrimido[5,4-d]pyrimidin-2-yl)(2- methoxyethyl)amino)methyl)benzoic acid | 718.816 |
| 336 | 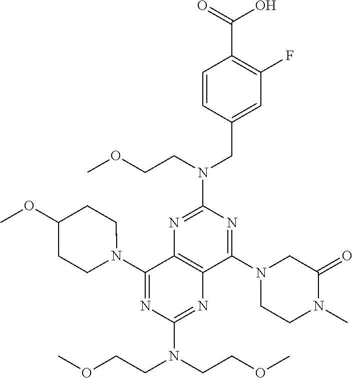 [SMILES: COCCN(Cc1ccc(C(=O)O)c(F)c1)c1nc(N2CCN(C)C(=O)C2)c2nc(N(CCOC)CCOC)nc(N3CCC(OC)CC3)c2n1] | 4-(((6-(bis(2-methoxyethyl)amino)-8-(4- methoxypiperidin-1-yl)-4-(4-methyl-3- oxopiperazin-1-yl)pyrimido[5,4-d]pyrimidin-2- yl)(2-methoxyethyl)amino)methyl)-2- fluorobenzoic acid | 713.8124 |
| 337 | 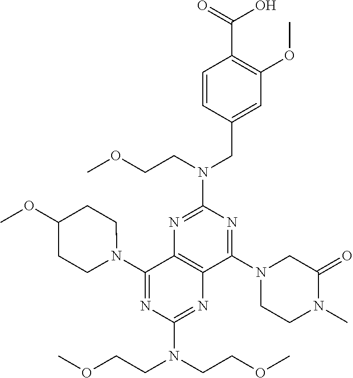 [SMILES: COCCN(Cc1ccc(C(=O)O)c(OC)c1)c1nc(N2CCN(C)C(=O)C2)c2nc(N(CCOC)CCOC)nc(N3CCC(OC)CC3)c2n1] | 4-(((6-(bis(2-methoxyethyl)amino)-8-(4- methoxypiperidin-1-yl)-4-(4-methyl-3- oxopiperazin-1-yl)pyrimido[5,4-d]pyrimidin-2- yl)(2-methoxyethyl)amino)methyl)-2- methoxybenzoic acid | 725.848 |
| 338 | 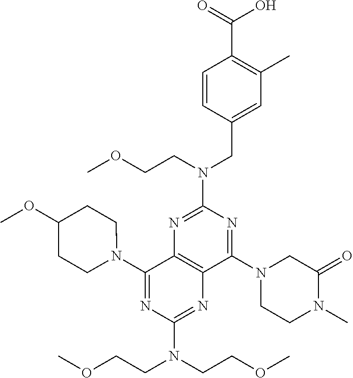 [SMILES: COCCN(Cc1ccc(C(=O)O)c(C)c1)c1nc(N2CCN(C)C(=O)C2)c2nc(N(CCOC)CCOC)nc(N3CCC(OC)CC3)c2n1] | 4-(((6-(bis(2-methoxyethyl)amino)-8-(4- methoxypiperidin-1-yl)-4-(4-methyl-3- oxopiperazin-1-yl)pyrimido[5,4-d]pyrimidin-2- yl)(2-methoxyethyl)amino)methyl)-2- methylbenzoic acid | 709.849 |
| 339 | 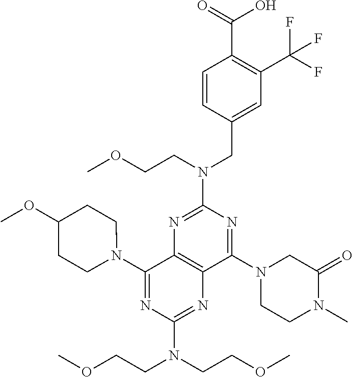 [SMILES: COCCN(Cc1ccc(C(=O)O)c(C(F)(F)F)c1)c1nc(N2CCN(C)C(=O)C2)c2nc(N(CCOC)CCOC)nc(N3CCC(OC)CC3)c2n1] | 4-(((6-(bis(2-methoxyethyl)amino)-8-(4- methoxypiperidin-1-yl)-4-(4-methyl-3- oxopiperazin-1-yl)pyrimido[5,4-d]pyrimidin-2- yl)(2-methoxyethyl)amino)methyl)-2- (trifluoromethyl)benzoic acid | 763.8202 |
| 340 | 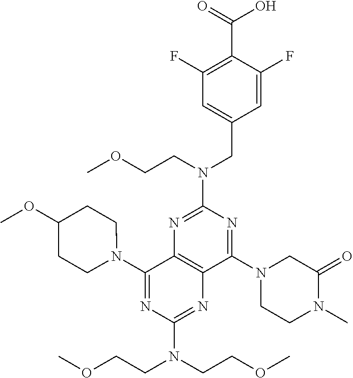 [SMILES: COCCN(Cc1cc(F)c(C(=O)O)c(F)c1)c1nc(N2CCN(C)C(=O)C2)c2nc(N(CCOC)CCOC)nc(N3CCC(OC)CC3)c2n1] | 4-(((6-(bis(2-methoxyethyl)amino)-8-(4- methoxypiperidin-1-yl)-4-(4-methyl-3- oxopiperazin-1-yl)pyrimido[5,4-d]pyrimidin-2- yl)(2-methoxyethyl)amino)methyl)-2,6- difluorobenzoic acid | 731.8028 |

and pharmaceutically acceptable salts and solvates thereof.

The compounds of Table 1 were named using ChemBioDraw® Ultra version 12.0 (PerkinElmer).

In one embodiment, the present invention also relates to salts, solvates, enantiomers, isomers (including optical, geometric and tautomeric isomers), polymorphs, multi-component complexes, liquid crystals, prodrugs of compounds of formula I and subformula thereof, and to isotopically-labeled compounds of formula I and subformula thereof.

In one embodiment, the present invention relates to enantiomers and isomers (including optical, geometric and tautomeric isomers) of compounds of formula I and subformula thereof. Indeed, the compounds of formula I and subformula thereof may contain an asymmetric center and thus may exist as different stereoisomeric forms. Accordingly, the present invention includes all possible stereoisomers and includes not only racemic compounds but the individual enantiomers and their non-racemic mixtures as well. When a compound is desired as a single enantiomer, such may be obtained by stereospecific synthesis, by resolution of the final product or any convenient Intermediate compound, or by chiral chromatographic methods as each are known in the art. Resolution of the final product, an Intermediate compound, or a starting material may be performed by any suitable method known in the art.

In one embodiment, the present invention also relates to salts of compounds of formula I and subformula thereof. Especially, the compounds of the invention may be in the form of pharmaceutically acceptable salts. Pharmaceutically acceptable salts of the compounds of formula I and subformula thereof include the acid addition and base salts thereof. Suitable acid addition salts are formed from acids which form non-toxic salts. Examples include the acetate, adipate, ammonium salt, aspartate, benzoate, besylate, benzenesulfonate, bicarbonate/carbonate, bisulphate/sulphate, bitartrate, borate, calcium edetate, camsylate, citrate, clavulanate, cyclamate, dihydrochloride, edetate, edisylate, estolate, esylate, formate, fumarate, gluceptate, gluconate, glucuronate, glutamate, glycollylarsanilate, hexafluorophosphate, hexylresorcinate, hibenzate, hydrabamine, hydrochloride/chloride, hydrobromide/bromide, hydroiodide/iodide, hydroxynaphthoate, isethionate, isothionate, lactate, lactobionate, laurate, malate, maleate, malonate, mandelate, mesylate, methylbromide, N-methylglucamine, methylnitrate, methylsulphate, mucate, panoate, naphthylate, 2-napsylate, nicotinate, nitrate, oleate, orotate, oxalate, palmitate, pamoate, pantothenate, phosphate/hydrogen phosphate/dihydrogen phosphate, polygalacturonate, pyroglutamate, saccharate, salicylate, stearate, subacetate, succinate, tannate, tartrate, teoclate, tosylate, triethiodide, trifluoroacetate, valerate and xinofoate salts. Preferred pharmaceutically acceptable acid addition salts include hydrochloride/chloride, hydrobromide/bromide, bisulphate/sulphate, nitrate, citrate, tosylate, esylate and acetate. Suitable base salts are formed from bases which form non-toxic salts. Examples include the aluminum, ammonia, arginine, benzathine, N-benzylphenethyl-amine, calcium, chloroprocaine, choline, N,N′-dibenzylethylene-diamine, diethanolamine, diethylamine, 2-(diethylamino)ethanol, diolamine, ethanolamine, ethylenediamine, glycine, lithium, lysine, magnesium, meglumine, N-methyl-glutamine, morpholine, 4-(2-hydroxyethyl)morpholine, olamine, ornithine, piperazine, potassium, procaine, sodium, tetramethylammonium hydroxide, tris(hydroxymethyl)aminomethane, tromethamine and zinc salts. Hemisalts of acids and bases may also be formed, for example, hemisulphate and hemicalcium salts. When the compounds of the invention contain a hydrogen-donating heteroatom (e.g. NH), the invention also covers salts and/or isomers formed by transfer of said hydrogen atom to a basic group or atom within the molecule.

Pharmaceutically acceptable salts of compounds of formula I and subformula thereof may be prepared by one or more of these methods:
(i) by reacting the compound of formula I with the desired acid;
(ii) by reacting the compound of formula I with the desired base;
(iii) by removing an acid- or base-labile protecting group from a suitable precursor of the compound of formula I or by ring-opening a suitable cyclic precursor, for example, a lactone or lactam, using the desired acid; or
(iv) by converting one salt of the compound of formula I to another by reaction with an appropriate acid or by means of a suitable ion exchange column.

All these reactions are typically carried out in solution. The salt may precipitate from solution and be collected by filtration or may be recovered by evaporation of the solvent. The degree of ionization in the salt may vary from completely ionized to almost non-ionized.

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of formula I above.

In one embodiment, the present invention also relates to solvates of compounds of formula I and subformula thereof. The compounds of the invention may be in the form of pharmaceutically acceptable solvates. Pharmaceutically acceptable solvates of the compounds of formula I and subformula thereof contains stoichiometric or sub-stoichiometric amounts of one or more pharmaceutically acceptable solvent molecule such as ethanol or water. The term “hydrate” refers to when the said solvent is water.

In one embodiment, the present invention also relates to prodrugs of compounds of formula I and subformula thereof. For example, in the case of an alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

Process of Manufacturing

The compounds of formula I can be prepared by different ways with reactions known by one skilled in the art.

The invention also provides a process of manufacturing of compounds of formula I:

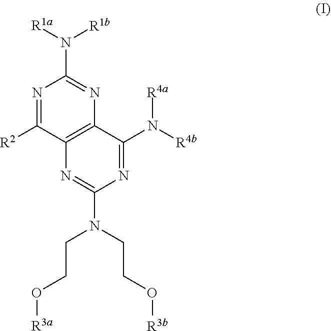

or a pharmaceutically acceptable salt or solvate thereof, wherein R^{1a}, R^{1b}, R², R^{3a}, R^{3b}, R^{4a} and R^{4b} are as defined above;

comprising the coupling of the intermediate compound of formula (A):

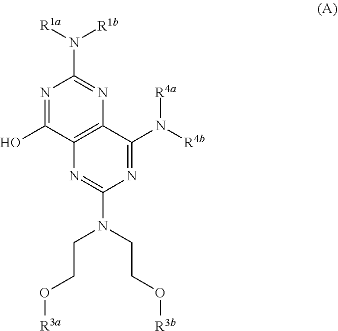

wherein R^{1a}, R^{1b}, R^{3a}, R^{3b}, R^{4a} and R^{4b} are as defined above;

with the amine (B) or the alcohol (C)

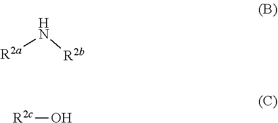

wherein R^{2a}, R^{2b} and R^{2c} are as defined above.

In one embodiment, the coupling is performed in presence of an activating agent, such as for example benzotriazol-1-yloxytris(dimethylamino)phosphonium hexafluorophosphate (BOP). Preferably, the coupling is also performed in presence of a catalyst, such as for example 1,8-diazabicyclo[5.4.0]undec-7-ene (DBU).

In another embodiment, the coupling is performed in presence of a base, such as for example diisopropylethylamine (DIEA) In one embodiment, the coupling is performed in a solvent such as dimethylformamide (DMF) or N-methylpyrolidone (NMP).

Uses

The invention is further directed to the use of the compounds of the invention, or pharmaceutically acceptable salts and solvates thereof, as inhibitors of ENT family transporters. Accordingly, in a particularly preferred embodiment, the invention relates to the use of compounds of formula I and subformula in particular those of Table 1 above, or pharmaceutically acceptable salts and solvates thereof, as inhibitors of ENT family transporters.

In one embodiment, the compounds of the invention are inhibitors of ENT1, ENT2, ENT3 and/or ENT4. In one embodiment, the compounds of the invention are inhibitors of ENT1 and ENT2. In one embodiment, the compounds of the invention are inhibitors of ENT1, preferably selective inhibitors of ENT1. In one embodiment, the compounds of the invention are inhibitors selective of ENT1, with respect to other ENT family transporters, especially with respect to ENT2 and ENT4.

The invention also provides a method for inhibiting ENT family transporters, especially ENT1, in a patient, preferably a warm-blooded animal, and even more preferably a human, in need thereof, which comprises administering to said patient an effective amount of a compound of the invention, or a pharmaceutically acceptable salt and solvate thereof.

The invention is further directed to the use of the compounds of the invention as a medicament, i.e. for medical use. Thus, in one embodiment, the invention provides the use of the compounds of the invention for the manufacturing of a medicament. Especially, the invention provides the use of the compounds of the invention for the manufacturing of a medicament.

Especially, the invention provides the compounds of the invention, for use in the treatment and/or prevention of proliferative disorders, including cancers. Thus, in one embodiment, the invention provides the use of the compounds of the invention for the manufacture of a medicament for treating and/or preventing cancer. The invention also provides a method of treatment of cancer, which comprises administering to a mammal species in need thereof a therapeutically effective amount of a compound of the invention.

The invention also provides for a method for delaying in patient the onset of cancer comprising the administration of a pharmaceutically effective amount of a compound of the invention to a patient in need thereof.

Various cancers are known in the art. Cancers that can be treated using the methods of the invention include solid cancers and non-solid cancers, especially benign and malignant solid tumors and benign and malignant non-solid tumors. The cancer may be metastatic or non-metastatic. The cancer may be may be familial or sporadic.

In one embodiment, the cancer to be treated according to the present invention is a solid cancer. As used herein, the term “solid cancer” encompasses any cancer (also referred to as malignancy) that forms a discrete tumor mass, as opposed to cancers (or malignancies) that diffusely infiltrate a tissue without forming a mass.

Examples of solid tumors include, but are not limited to: biliary tract cancer, brain cancer (including glioblastomas and medulloblastomas), breast cancer, carcinoid, cervical cancer, choriocarcinoma, colon cancer, colorectal cancer, endometrial cancer, esophageal cancer, gastric cancer, glioma, head and neck cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumor), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumors (seminomas, and non-seminomas such as teratomas and choriocarcinomas), stromal tumors, germ cell tumors, thyroid cancer (including thyroid adenocarcinoma and medullary carcinoma) and urothelial cancer.

In another embodiment, the cancer to be treated according to the present invention is a non-solid cancer. Examples of non-solid tumors include but are not limited to hematological neoplasms. As used herein, a hematologic neoplasm is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas). Chronic lymphoid leukemias include, for example, T cell chronic lymphoid leukemias and B cell chronic lymphoid leukemias.

In a specific embodiment, the cancer is selected from breast, carcinoid, cervical, colorectal, endometrial, glioma, head and neck, liver, lung, melanoma, ovarian, pancreatic, prostate, renal, gastric, thyroid and urothelial cancers.

In a specific embodiment, the cancer is breast cancer. In a specific embodiment, the cancer is carcinoid cancer. In a specific embodiment, the cancer is cervical cancer. In a specific embodiment, the cancer is colorectal cancer. In a specific embodiment, the cancer is endometrial cancer. In a specific embodiment, the cancer is glioma. In a specific embodiment, the cancer is head and neck cancer. In a specific embodiment, the cancer is liver cancer. In a specific embodiment, the cancer is lung cancer. In a specific embodiment, the cancer is melanoma. In a specific embodiment, the cancer is ovarian cancer. In a specific embodiment, the cancer is pancreatic cancer. In a specific embodiment, the cancer is prostate cancer. In a specific embodiment, the cancer is renal cancer. In a specific embodiment, the cancer is gastric cancer. In a specific embodiment, the cancer is thyroid cancer. In a specific embodiment, the cancer is urothelial cancer.

In another specific embodiment, the cancer is selected from the group consisting of: leukemia and multiple myeloma.

Preferably, the patient is a warm-blooded animal, more preferably a human.

In one embodiment, the subject receiving the ENT inhibitor of the invention is treated with an additional therapeutic agent in combination with the ENT inhibitor of the invention, or has received the additional therapeutic agent within about fourteen days of administration of the ENT inhibitor of the invention. In one embodiment, the additional therapeutic agent comprises an adenosine receptor antagonist.

In one embodiment, the subject has previously received at least one prior therapeutic treatment, and has progressed subsequent to the administration of the at least one prior therapeutic treatment and prior to administration of the ENT inhibitor of the invention.

In one embodiment, the prior therapeutic treatment is selected from the group consisting of chemotherapy, immunotherapy, radiation therapy, stem cell transplant, hormone therapy, and surgery.

In one embodiment, ENT inhibitor of the invention is administered prior to, concomitant with, or subsequent to administration of the additional therapeutic agent, such as an adenosine receptor antagonist.

Formulations

The invention also provides pharmaceutical compositions comprising a compound of formula I and subformula thereof, or a pharmaceutically acceptable salt and solvate thereof, and at least one pharmaceutically acceptable carrier, diluent, excipient and/or adjuvant.

Another object of this invention is a medicament comprising at least one compound of the invention, or a pharmaceutically acceptable salt and solvate thereof, as active ingredient.

Generally, for pharmaceutical use, the compounds of the invention may be formulated as a pharmaceutical preparation comprising at least one compound of the invention and at least one pharmaceutically acceptable carrier, diluent, excipient and/or adjuvant, and optionally one or more further pharmaceutically active compounds. Details regarding the presence of further pharmaceutically active compounds are provided hereafter.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use.

Depending on the condition to be prevented or treated and the route of administration, the active compound of the invention may be administered as a single daily dose, divided over one or more daily doses, or essentially continuously, e.g. using a drip infusion.

Combined Use with Adenosine Receptor Antagonist

The invention further relates to the combined use of an ENT inhibitor of the invention, of formula I or a subformula thereof, as defined above, with an adenosine receptor antagonist.

The invention thus relates to a combination comprising:
(a) an effective amount of an ENT inhibitor of the invention, of formula I or a subformula thereof, as defined above; and
(b) an effective amount of an adenosine receptor antagonist.

In the context of the present invention the term “combination” preferably means a combined occurrence of the ENT inhibitor and of an A2AR antagonist. Therefore, the combination of the invention may occur either as one composition, comprising all the components in one and the same mixture (e.g. a pharmaceutical composition), or may occur as a kit of parts, wherein the different components form different parts of such a kit of parts. The administration of the ENT inhibitor and of the A2AR antagonist may occur either simultaneously or timely staggered, with similar or different timing of administration (i.e. similar or different numbers of administration of each component), either at the same site of administration or at different sites of administration, under similar of different dosage form.

The invention further relates to a method of treating cancer, comprising: administering, to a patient in need thereof, a combination of an adenosine receptor antagonist and the ENT inhibitor of the invention.

Above embodiments relative to the ENT inhibitors of the invention also apply to the combination of the invention. Especially, in one embodiment, in the combination of the invention, the ENT inhibitor may be of formula I or of the sub-formulae defined above.

As a second component, the combination of the invention includes at least one adenosine receptor antagonist.

As defined above, “adenosine receptor antagonist” refers to a compound that, upon administration to a patient, results in inhibition or down-regulation of a biological activity associated with activation of an adenosine receptor in the patient, including any of the downstream biological effects otherwise resulting from the binding to an adenosine receptor of its natural ligand. Such adenosine receptor antagonists include any agent that can block activation of an adenosine receptor or any of the downstream biological effects of an adenosine receptor activation.

Adenosine receptors (or P1 receptors) are a class of purinergic G protein-coupled receptors with adenosine as endogenous ligand. There are four known types of adenosine receptors in humans: A1, A2A, A2B and A3; each is encoded by a different gene (ADOARA1, ADORA2A, ADORA2B, and ADORA3 respectively).

In one embodiment, the adenosine receptor antagonist is an antagonist of A1 receptor, A2A receptor, A2B receptor, A3 receptor or of a combination thereof.

In one embodiment, the adenosine receptor antagonist is an antagonist of A2A receptor, A2B receptor or of a combination thereof. In one embodiment, the adenosine receptor antagonist is an A2A or A2B receptor antagonist.

In one embodiment, the adenosine receptor antagonist is an antagonist of A2A receptor (A2AR antagonist). In one embodiment, the adenosine receptor antagonist is an antagonist of A2B receptor (A2BR antagonist).

In one embodiment, the adenosine receptor antagonist is an antagonist which is selective of A2A receptor with respect to other adenosine receptors. In one embodiment, the adenosine receptor antagonist is an antagonist which is selective of A2A receptor with respect to A2B receptor.

In one embodiment, the adenosine receptor antagonist is an antagonist which is selective of A2B receptor with respect to other adenosine receptors. In one embodiment, the adenosine receptor antagonist is an antagonist which is selective of A2B receptor with respect to A2A receptor.

In a specific embodiment, the combination of the invention comprises at least one A2A receptor antagonist as herein defined and at least one ENT inhibitor of formula I as defined above.

A2A Receptor Antagonist

In one embodiment, the combination of the invention includes at least one A2AR antagonist.

An “A2AR antagonist” refers to a compound that, upon administration to a patient, results in inhibition or down-regulation of a biological activity associated with activation of A2A receptor in the patient, including any of the downstream biological effects otherwise resulting from the binding to A2A receptor of its natural ligand. Such A2AR antagonists include any agent that can block activation of A2A receptor or any of the downstream biological effects of A2A receptor activation.

Examples of A2AR antagonists include: Preladenant (SCH-420,814), Vipadenant (BIIB-014), Tozadenant (SYK-115), ATL-444, Istradefylline (KW-6002), MSX-3, SCH-58261, SCH-412,348, SCH-442,416, ST-1535, Caffeine, VER-6623, VER-6947, VER-7835, ZM-241,385, theophylline. It also includes A2AR antagonists disclosed in WO2018/178338, WO2011/121418, WO2009/156737, WO2011/095626 or WO2018/136700, the content of which is herein incorporated by reference.

In one embodiment, the A2AR antagonist is a thiocarbamate derivative, especially a thiocarbamate derivative as those disclosed in WO2018/178338. More preferably the A2AR antagonist is a thiocarbamate derivative of formula (II) as described below.

Thus, in a specific embodiment, the invention provides a combination comprising:
(a) an ENT inhibitor according to the invention, of formula I or a subformula thereof, as defined above; and
(b) an A2AR antagonist being a thiocarbamate derivative of Formula (II), corresponding to compounds of Formula (I) of WO2018/178338:

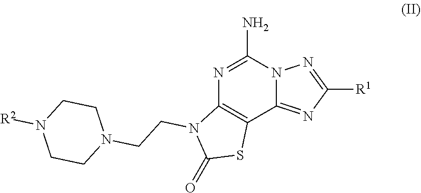

or a pharmaceutically acceptable salt or solvate thereof, wherein R¹ and R² are as defined below.

In a preferred embodiment, the A2AR antagonist is thus a compound of Formula (II), or a pharmaceutically acceptable salt or solvate thereof, wherein:
R¹ represents 5- or 6-membered heteroaryl or 5- or 6-membered aryl, wherein heteroaryl or aryl groups are optionally substituted by one or more substituent selected from C₁-C₆ alkyl (preferably methyl) and halo (preferably fluoro or chloro); preferably R¹ represents 5-membered heteroaryl; more preferably R¹ represents furyl;
R² represents 6-membered aryl or 6-membered heteroaryl,
   wherein heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino and alkylsulfonealkyl;
      said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl alkylsulfonyl and alkylsulfonealkyl;
   or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, preferred A2AR antagonists of Formula (II) are of Formula (IIa):

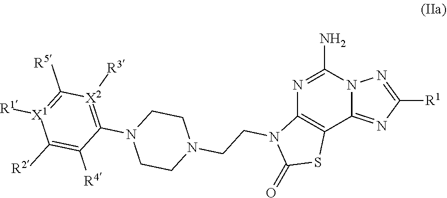

or a pharmaceutically acceptable salt or solvate thereof, wherein:
R¹ represents 5- or 6-membered heteroaryl or 5- or 6-membered aryl, wherein heteroaryl or aryl groups are optionally substituted by one or more substituent selected from C₁-C₆ alkyl (preferably methyl) and halo (preferably fluoro or chloro); preferably R¹ represents 5-membered heteroaryl; more preferably R¹ represents furyl;
X¹ and X² represent each independently C or N;
R^{1′} is absent when X¹ is N; or when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl;
   said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
   R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl;
      said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
   or R^{1′} and R^{2′} form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
   R^{3′} is absent when X² is N; or when X² is C, R^{3′} represents H or halo, preferably H or F;
   R^{4′} represents H or halo, preferably H or F; and
   R^{5′} represents H or halo, preferably H or F.

In one embodiment, preferred A2AR antagonists of Formula (IIa) are those of Formula (Ia-1):

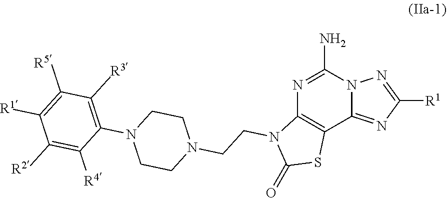

or a pharmaceutically acceptable salt or solvate thereof, wherein R¹, R^{1′}, R^{2′}, R^{3′}, R^{4′} and R^{5′} are as defined in Formula (IIa).

In one embodiment, preferred A2AR antagonists of Formula (IIa-1) are those of Formula (IIa-1a):

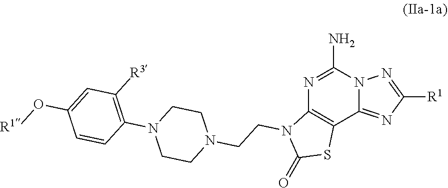

or a pharmaceutically acceptable salt or solvate thereof, wherein:
R¹ and R^{3′} are as defined in Formula (ha); and
R¹″ represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, preferred A2AR antagonists of Formula (IIa-1) are those of Formula (IIa-1b):

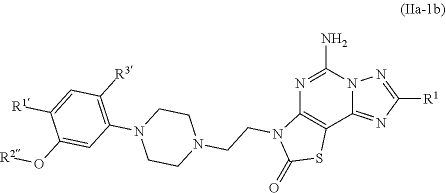

or a pharmaceutically acceptable salt or solvate thereof, wherein:
R¹ and R^{3′} are as defined in Formula (IIa);
R^{1′} represents H or halo, preferably H or F; and
R²″ represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, preferred A2AR antagonists of Formula (IIa-1) are those of Formula (IIa-1c) or (IIa-1d):

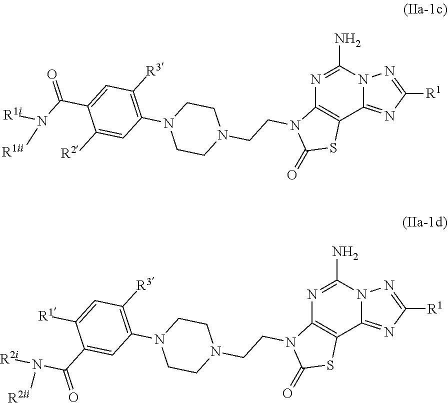

or a pharmaceutically acceptable salt or solvate thereof, wherein:
R¹ and R^{3′} are as defined in Formula (Ia);
R^{1′} represents H or halo, preferably H or F;
R^{2′} represents H or halo, preferably H or F;
R¹ⁱ and R¹ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl; and
R²ⁱ and R²ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl.

In one embodiment, preferred A2AR antagonists of Formula (IIa) are those of Formulae (IIa-2) or (IIa-3):

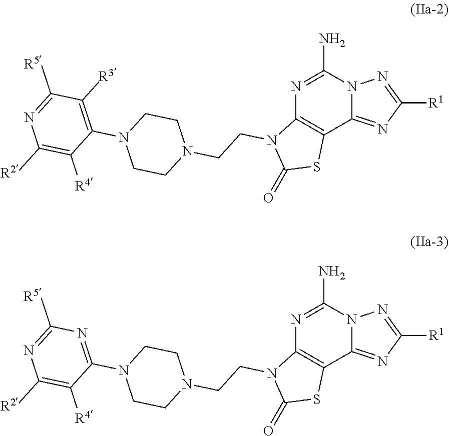

or a pharmaceutically acceptable salt or solvate thereof, wherein R¹, R^{2′}, R^{3′}, R^{4′} and R^{5′} are as defined in Formula (IIa).

Particularly preferred A2AR antagonists of Formula (II) are those listed hereafter:
3-(2-(4-(4-((1H-1,2,3-triazolo-4yl)methoxy-2fluorophenyl)piperazine-1-yl)ethyl)-5-amino-(8-(furan-2-yl)thiazolo[5,4-e][1,2,4]triazolo[1,5-c]pyrimidine-2(3H)-one
5-((4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)methyl)-1,3,4-oxadiazol-2(3H)-one
5-amino-3-(2-(4-(3-fluoropyridin-4-yl)piperazin-1-yl)ethyl)-8-(furan-2-yl)thiazolo[5,4-e][1,2,4]triazolo[1,5-c]pyrimidin-2(3H)-one
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)acetamide
(S)-5-amino-3-(2-(4-(2-fluoro-4-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one
(R)-5-amino-3-(2-(4-(2-fluoro-4-(2-(methylsulfinyl)ethoxy)phenyl)-piperazin-1-yl)ethyl)-8-(furan-2-yl)thiazolo[5,4-e][1,2,4]triazolo[1,5-c]pyrimidin-2(3H)-one
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one
5-amino-8-(furan-2-yl)-3-(2-(4-(4-(2-hydroxyethoxy) phenyl)piperazin-1-yl)ethyl)thiazolo[5,4-e][1,2,4]triazolo[1,5-c]pyrimidin-2(3H)-one
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)phenoxy)acetic acid
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)phenoxy)acetamide
5-amino-3-(2-(4-(4-(2,3-dihydroxypropoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(4-(2-aminoethoxy)phenyl)piperazin-1-yl)ethyl)-8-(furan-2-yl)thiazolo[5,4-e][1,2,4]triazolo[1,5-c]pyrimidin-2(3H)-one
4-(4-(2-(5-amino-8-(furan-2-yl)-2-oxothiazolo[5,4-e][1,2,4]triazolo[1,5-c]pyrimidin-3(2H)-yl) ethyl)piperazin-1-yl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-methylbenzamide
5-amino-8-(furan-2-yl)-3-(2-(4-(4-(2-morpholinoethoxy)phenyl)piperazin-1-yl)ethyl)thiazolo[5,4-e][1,2,4]triazolo[1,5-c]pyrimidin-2(3H)-one
5-amino-3-(2-(4-(4-(2-(dimethylamino)ethoxy)phenyl)piperazin-1-yl)ethyl)-8-(furan-2-yl)thiazolo[5,4-e][1,2,4]triazolo[1,5-c]pyrimidin-2(3H)-one
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzenesulfonamide
4-(4-(2-(5-amino-8-(furan-2-yl)-2-oxothiazolo[5,4-e][1,2,4]triazolo[1,5-c]pyrimidin-3(2H)-yl)ethyl) piperazin-1-yl)-N-methylbenzenesulfonamide
5-amino-8-(furan-2-yl)-3-(2-(4-(4-(methylsulfonyl) phenyl)piperazin-1-yl)ethyl)thiazolo[5,4-e][1,2,4]triazolo[1,5-c]pyrimidin-2(3H)-one
5-amino-8-(furan-2-yl)-3-(2-(4-(4-(methylsulfinyl) phenyl)piperazin-1-yl)ethyl)thiazolo[5,4-e][1,2,4]triazolo[1,5-c]pyrimidin-2(3H)-one
3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzamide
5-amino-8-(furan-2-yl)-3-(2-(4-(3-(2-hydroxyethoxy) phenyl)piperazin-1-yl)ethyl)thiazolo[5,4-e][1,2,4]triazolo[1,5-c]pyrimidin-2(3H)-one
5-amino-3-(2-(4-(2-fluoro-4-(2-oxo-2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(piperidin-4-ylmethoxy) phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(piperazine-1-carbonyl) phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(piperazin-1-ylsulfonyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(methylsulfonyl)phenyl) piperazin-1-yl)ethyl)-8-(furan-2-yl)thiazolo[5,4-e][1,2,4]triazolo[1,5-c]pyrimidin-2(3H)-one
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-aminoethyl)-3-fluorobenzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylamino)ethyl) benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluorobenzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-hydroxyethyl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-3-fluorobenzamide
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)acetic acid
2-(4-(4-(2-(5-amino-8-(furan-2-yl)-2-oxothiazolo[5,4-e][1,2,4]triazolo[1,5-c]pyrimidin-3(2H)-yl)ethyl) piperazin-1-yl)-3,5-difluorophenoxy) acetic acid
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid
(S)-2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)propanoic acid
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-2-methylpropanoic acid
3-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenyl)propanoic acid
4-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)butanoic acid
2-(3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,6-difluorophenoxy) acetic acid
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) acetic acid
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorobenzoic acid
2-((2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)ethyl) amino)acetamide
2-((2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)ethyl)(methyl)amino)acetamide
5-amino-3-(2-(4-(2-fluoro-4-(piperidin-4-yloxy) phenyl)piperazin-1-yl) ethyl)-8-(furan-2-yl) thiazolo[5,4-e][1,2,4]triazolo[1,5-c]pyrimidin-2(3H)-one
5-amino-3-(2-(4-(2-fluoro-4-(pyrrolidin-3-yloxy)phenyl)piperazin-1-yl)ethyl)-8-(furan-2-yl)thiazolo[5,4-e][1,2,4]triazolo[1,5-c]pyrimidin-2(3H)-one
3-(2-(4-(4-((1H-1,2,4-triazol-3-yl)methoxy)-2-fluorophenyl)piperazin-1-yl)ethyl)-5-amino-8-(furan-2-yl)thiazolo [5,4-e][1,2,4]triazolo[1,5-c]pyrimidin-2(3H)-one
2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)-N-(2-(methylamino)ethyl) acetamide
2-(4-(4-(2-(5-amino-8-(furan-2-yl)-2-oxothiazolo [5,4-e][1,2,4]triazolo[1,5-c]pyrimidin-3(2H)-yl) ethyl)piperazin-1-yl)-3-fluorophenoxy)-N-(2-(dimethylamino)ethyl) acetamide
2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)-N-(2-aminoethyl)acetamide
(R)-2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)propanoic acid
2-(4-(4-(2-(5-amino-8-(furan-2-yl)-2-oxothiazolo [5,4-e][1,2,4]triazolo[1,5-c]pyrimidin-3(2H)-yl) ethyl)piperazin-1-yl)-3-fluorophenoxy)acetamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-methyl-N-(2-(methylamino)ethyl) benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluoro-N-methylbenzamide
(R)-4-(4-(2-(5-amino-8-(furan-2-yl)-2-oxothiazolo [5,4-e][1,2,4]triazolo[1,5-c]pyrimidin-3(2H)-yl) ethyl)piperazin-1-yl)-N-(1-(dimethylamino) propan-2-yl)-3-fluorobenzamide
2-(4-(4-(2-(5-amino-8-(furan-2-yl)-2-oxothiazolo [5,4-e][1,2,4]triazolo[1,5-c]pyrimidin-3(2H)-yl) ethyl)piperazin-1-yl)-3-fluorophenoxy)-N-methyl-N-(2-(methylamino)ethyl) acetamide
2-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy)-2-methylpropanoic acid
(S)-2-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy) propanoic acid
(R)-2-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy) propanoic acid
2-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy)-N-(2-(methylamino)ethyl) acetamide
2-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy)-N-(2-(dimethylamino)ethyl) acetamide
5-(4-(2-(5-amino-8-(furan-2-yl)-2-oxothiazolo[5,4-e][1,2,4]triazolo [1,5-c]pyrimidin-3(2H)-yl)ethyl)piperazin-1-yl)-N-(2-(dimethylamino)ethyl)-2,4-difluoro-N-methylbenzamide
4-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy) butanoic acid
3-(2-(4-(5-((1H-tetrazol-5-yl)methoxy)-2,4-difluorophenyl)piperazin-1-yl)ethyl)-5-amino-8-(furan-2-yl)thiazolo[5,4-e][1,2,4]triazolo[1,5-c]pyrimidin-2(3H)-one
5-amino-3-(2-(4-(2-fluoro-4-((1-methyl-1H-1,2,4-triazol-3-yl)methoxy) phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-((1-methyl-1H-1,2,4-triazol-3-yl) methoxy)phenyl)piperazin-1-yl)ethyl)-8-(furan-2-yl)thiazolo [5,4-e][1,2,4]triazolo[1,5-c]pyrimidin-2(3H)-one
4-(4-(2-(5-amino-8-(furan-2-yl)-2-oxothiazolo[5,4-e][1,2,4]triazolo[1,5-c]pyrimidin-3(2H)-yl) ethyl)piperazin-1-yl)-3-fluoro-N-(2-(methyl (oxetan-3-yl)amino)ethyl) benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-((2-hydroxyethyl)amino)ethyl)benzamide
2-amino-N-(2-(4-(4-(2-(5-amino-8-(furan-2-yl)-2-oxothiazolo [5,4-e][1,2,4]triazolo[1,5-c] pyrimidin-3(2H)-yl) ethyl)piperazin-1-yl)-3-fluorophenoxy)ethyl) acetamide
(S)-2-amino-N-(2-(4-(4-(2-(5-amino-8-(furan-2-yl)-2-oxothiazolo [5,4-e][1,2,4]triazolo [1,5-c]pyrimidin-3(2H)-yl)ethyl)piperazin-1-yl)-3-fluorophenoxy)ethyl)-3-methylbutanamide
ethyl 2-(5-(4-(2-(5-amino-8-(furan-2-yl)-2-oxothiazolo[5,4-e][1,2,4]triazolo[1,5-c]pyrimidin-3(2H)-yl) ethyl)piperazin-1-yl)-2,4-difluorophenoxy) acetate
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) acetonitrile
5-amino-8-(furan-2-yl)-3-(2-(4-(pyridin-4-yl) piperazin-1-yl)ethyl)thiazolo[5,4-e][1,2,4]triazolo[1,5-c]pyrimidin-2(3H)-one
5-amino-8-(furan-2-yl)-3-(2-(4-(pyrimidin-4-yl)piperazin-1-yl)ethyl)thiazolo[5,4-e][1,2,4]triazolo[1,5-c]pyrimidin-2(3H)-one
5-amino-3-(2-(4-(2,4-difluoro-5-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(6-fluoro-2-oxoindolin-5-yl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(S-methylsulfonimidoyl)phenyl)piperazin-1-yl)ethyl)-8-(furan-2-yl)thiazolo[5,4-e][1,2,4]triazolo[1,5-c]pyrimidin-2(3H)-one
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluorobenzamide
5-amino-3-(2-(4-(5-fluoro-2-methylpyridin-4-yl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3R,4R)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3S,4S)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(2-hydroxy-2-methylpropoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(2-hydroxypropan-2-yl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(3,3,3-trifluoro-2-hydroxypropoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-5-(2-hydroxyethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(2,4-difluoro-5-((2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
(R)-5-amino-3-(2-(4-(2,4-difluoro-5-((2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-morpholinoethyl)acetamide
5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluoro-N-(morpholin-3-ylmethyl)benzamide
5-amino-3-(2-(4-(2-fluoro-4-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3R,4R)-4-fluoropyrrolidin-3-yl)oxy)phenyl)piperazin-1-yl)ethyl)-8-(furan-2-yl)thiazolo[5,4-e][1,2,4]triazolo [1,5-c]pyrimidin-2(3H)-one
5-amino-3-(2-(4-(2-fluoro-4-(((3S,4S)-4-fluoropyrrolidin-3-yl)oxy)phenyl)piperazin-1-yl)ethyl)-8-(furan-2-yl)thiazolo[5,4-e][1,2,4]triazolo [1,5-c]pyrimidin-2(3H)-one
5-amino-3-(2-(4-(2-fluoro-4-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-morpholinoethyl)acetamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-morpholinoethyl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(morpholin-3-ylmethyl)benzamide
5-amino-3-(2-(4-(4-(azetidin-3-yloxy)phenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one
(R)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide
5-amino-3-(2-(4-(2,4-difluoro-5-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one
(R)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one
(S)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide
(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide
5-amino-3-(2-(4-(2-fluoro-4-(1-oxidothiomorpholine-4-carbonyl)phenyl)piperazin-1-yl)ethyl)-8-(furan-2-yl)thiazolo [5,4-e][1,2,4]triazolo [1,5-c]pyrimidin-2(3H)-one
5-amino-3-(2-(4-(2-fluoro-4-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo [1,5-c]pyrimidin-2(3H)-one
(R)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one
(S)-5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-N-(2,3-dihydroxypropyl)-2,4-difluorobenzamide
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide
5-amino-3-(2-(4-(4-(azetidin-3-yloxy)-2-fluorophenyl)piperazin-1-yl)ethyl)-8-(furan-2-yl)thiazolo[5,4-e][1,2,4]triazolo[1,5-c]pyrimidin-2(3H)-one
5-amino-3-(2-(4-(5-(azetidin-3-yloxy)-2,4-difluorophenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(2,4-difluoro-5-(3-(methylsulfinyl)propoxy)phenyl)piperazin-1-yl)ethyl)-8-(furan-2-yl)thiazolo[5,4-e][1,2,4]triazolo[1,5-c]pyrimidin-2(3H)-one

In one embodiment, the A2AR antagonist of Formula (II) is selected from:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 7);
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo [1,5-c]pyrimidin-2(3H)-one (compound 8a) and
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo [1,5-c]pyrimidin-2(3H)-one (compound 8b).

In a specific embodiment, the A2AR antagonist of Formula (II) is selected from:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 7); and
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo [1,5-c]pyrimidin-2(3H)-one (compound 8a).

In preferred embodiment, the A2AR antagonist of Formula (II) is (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8a).

In another preferred embodiment, the A2AR antagonist of Formula (II) is (−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8b).

The embodiments relative to salts, solvates, enantiomers, isomers (including optical, geometric and tautomeric isomers), polymorphs, multi-component complexes, liquid crystals, prodrugs and isotopically-labeled ENT inhibitors of the invention also apply to the A2AR antagonists Formula (II) and subformula thereof detailed above

In another embodiment, the A2AR antagonist is an A2AR antagonist disclosed in WO2011/121418. Especially, the A2AR antagonist is the compound of example 1 of WO2011/121418, namely 5-bromo-2,6-di-(1H-pyrazol-1-yl)pyrimidin-4-amine, also known as NIR178:

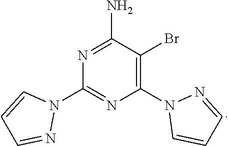 [SMILES: Nc1nc(-n2cccn2)nc(-n2cccn2)c1Br]

In another embodiment, the A2AR antagonist is an A2AR antagonist disclosed in WO2009/156737. Especially, the A2AR antagonist is the compound of example 1S of WO2009/156737, namely (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine, also known as CPI-444:

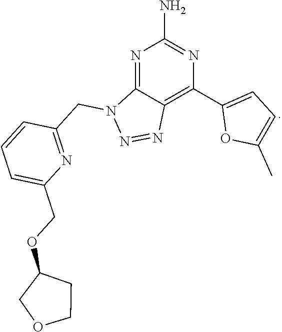 [SMILES: Cc1ccc(-c2nc(N)nc3c2nnn3Cc2cccc(CO[C@H]3CCOC3)n2)o1]

In another embodiment, the A2AR antagonist is an A2AR antagonist disclosed in WO2011/095626. Especially, the A2AR antagonist is the compound (cxiv) of WO2011/095626, namely 6-(2-chloro-6-methylpyridin-4-yl)-5-(4-fluorophenyl)-1,2,4-triazin-3-amine, also known as AZD4635:

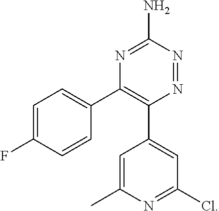 [SMILES: Cc1cc(-c2nnc(N)nc2-c2ccc(F)cc2)cc(Cl)n1]

In another embodiment, the A2AR antagonist is an A2AR antagonist disclosed in WO2018/136700. Especially, the A2AR antagonist is the compound of example 1 of WO2018/136700, namely 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile, also known as AB928:

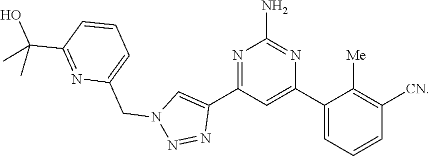 [SMILES: Cc1c(C#N)cccc1-c1cc(-c2cn(Cc3cccc(C(C)(C)O)n3)nn2)nc(N)n1]

In another embodiment, the A2AR antagonist is Preladenant (SCH-420,814), namely 2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine:

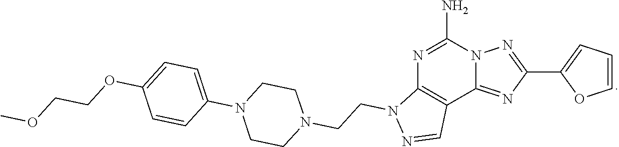 [SMILES: COCCOc1ccc(N2CCN(CCn3ncc4c3nc(N)n3nc(-c5ccco5)nc43)CC2)cc1]

In another embodiment, the A2AR antagonist is Vipadenant (BIIB-014), namely 3-(4-amino-3-methylbenzyl)-7-(2-furyl)-3H-(1,2,3)triazolo(4,5-d)pyrimidine-5-amine:

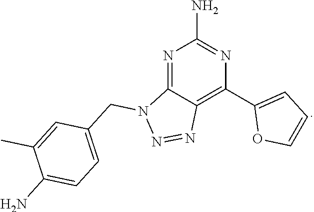 [SMILES: Cc1cc(Cn2nnc3c(-c4ccco4)nc(N)nc32)ccc1N]

In another embodiment, the A2AR antagonist is Tozadenant (SYK-115), namely 4-hydroxy-N-(4-methoxy-7-morpholinobenzo[d]thiazol-2-yl)-4-methylpiperidine-1-carboxamide:

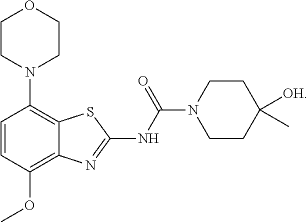 [SMILES: COc1ccc(N2CCOCC2)c2sc(NC(=O)N3CCC(C)(O)CC3)nc12]

Thus, in one embodiment, the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide.

In one embodiment, the adenosine receptor antagonist is 5-bromo-2,6-di-(1H-pyrazol-1-yl)pyrimidin-4-amine. In one embodiment, the adenosine receptor antagonist is (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine. In one embodiment, the adenosine receptor antagonist is 6-(2-chloro-6-methylpyridin-4-yl)-5-(4-fluorophenyl)-1,2,4-triazin-3-amine. In one embodiment, the adenosine receptor antagonist is 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile.

A2B Receptor Antagonist

In one embodiment, the combination of the invention includes at least one A2BR antagonist.

An “A2BR antagonist” refers to a compound that, upon administration to a patient, results in inhibition or down-regulation of a biological activity associated with activation of A2B receptor in the patient, including any of the downstream biological effects otherwise resulting from the binding to A2B receptor of its natural ligand. Such A2BR antagonists include any agent that can block activation of A2B receptor or any of the downstream biological effects of A2B receptor activation.

Examples of A2BR antagonists include: Vipadenant (BIIB-014), CVT-6883, MRS-1706, MRS-1754, PSB-603, PSB-0788, PSB-1115, OSIP-339,391, ATL-801, theophylline, Caffeine,

Specific Combinations

In one embodiment, the combination of the invention comprises:
(a) an effective amount of an ENT inhibitor of the invention, of formula I or a subformula thereof, and
(b) an effective amount of an adenosine receptor antagonist, preferably an A2AR antagonist, preferably selected from:
   (+)-5-amino-3-(2-(4-(2,4-difluoro-5-(2-(methylsulfinyl)ethoxy)phenyl)piperazin-1-yl)ethyl)-8-(furan-2-yl)thiazolol[5,4-e][1,2,4]triazolo[1,5-c]pyrimidin-2(3H)-one;
   (−)-5-amino-3-(2-(4-(2,4-difluoro-5-(2-(methylsulfinyl)ethoxy)phenyl)piperazin-1-yl)ethyl)-8-(furan-2-yl)thiazolol[5,4-e][1,2,4]triazolo[1,5-c]pyrimidin-2(3H)-one;
   5-bromo-2,6-di-(1H-pyrazol-1-yl)pyrimidin-4-amine;
   (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
   6-(2-chloro-6-methylpyridin-4-yl)-5-(4-fluorophenyl)-1,2,4-triazin-3-amine;
   3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
   and pharmaceutically acceptable salts thereof.

In one embodiment, the combination of the invention comprises:
(a) an effective amount of an ENT inhibitor of the invention, of formula I or a subformula thereof, and
(b) an effective amount of (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one as A2AR antagonist.

In one embodiment, the combination of the invention comprises:
(a) an effective amount of an ENT inhibitor of the invention, of formula I or a subformula thereof, and
(b) an effective amount of (−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one as A2AR antagonist.

Combined Formulation and Kit of Parts

The invention further provides a combined formulation, comprising the combination of the invention. Especially, the invention provides a combined formulation, comprising: an effective amount of an adenosine receptor antagonist in combination with an effective amount of an ENT inhibitor of the invention, as defined above, along with a pharmaceutically acceptable excipient.

The invention further relates to a combined pharmaceutical composition comprising the combination of the invention. In one embodiment, the pharmaceutical composition comprises:
(a) an effective amount of an ENT inhibitor of the invention, of formula I or a subformula thereof, as defined above;
(b) an effective amount of an adenosine receptor antagonist; and
(c) at least one pharmaceutically acceptable carrier, diluent, excipient and/or adjuvant.

The specific embodiments relative to the adenosine receptor antagonists and to the ENT inhibitor of the invention recited above also apply in the context of the combined formulation and pharmaceutical composition of the invention.

In a preferred embodiment, the invention provides a combined pharmaceutical composition comprising:
(a) an effective amount of an ENT inhibitor of the invention, of formula I or a subformula thereof, as defined above;
(b) an effective amount an A2AR antagonist being a thiocarbamate derivative, more preferably a thiocarbamate derivative of Formula (II)

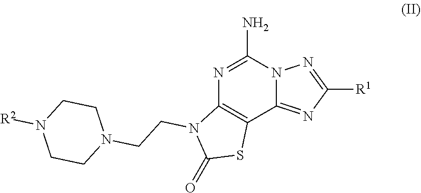

or a pharmaceutically acceptable salt or solvate thereof, as defined above; and
(c) at least one pharmaceutically acceptable carrier, diluent, excipient and/or adjuvant.

In one embodiment, the combined formulation or the pharmaceutical composition of the invention further comprises an additional therapeutic agent.

The at least one pharmaceutically acceptable carrier, diluent, excipient and/or adjuvant for use in the preparation of the administration forms will be clear to the skilled person; reference is made to the latest edition of Remington's Pharmaceutical Sciences. The specific embodiments relative to formulations comprising an ENT inhibitor of the invention also apply in the context of the combined formulation and pharmaceutical composition of the invention.

The invention further relates to a kit of parts comprising the combination of the invention. In one embodiment, the kit of parts of the invention comprises:
(a) a first part comprising an effective amount of an ENT inhibitor of the invention, of formula I or a subformula thereof, as defined above; and
(b) a second part comprising an effective amount of an adenosine receptor antagonist.

Above embodiments relative to the ENT inhibitor of the invention and adenosine receptor antagonists also apply to the kit of parts of the invention.

In a preferred embodiment, the invention provides a kit of parts comprising:
(a) a first part comprising an effective amount of an ENT inhibitor of the invention, of formula I or a subformula thereof, as defined above; and
(b) a second part comprising an effective amount an A2AR antagonist being a thiocarbamate derivative, more preferably a thiocarbamate derivative of Formula (II)

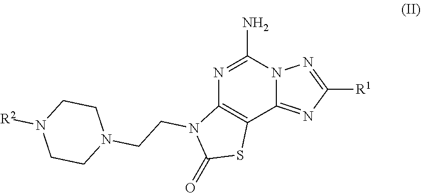

or a pharmaceutically acceptable salt or solvate thereof, as defined above.

Depending on the ENT inhibitor and adenosine receptor antagonist, the first and second parts of the kit may be under the form of pharmaceutical compositions. Excipients, dosage form and administration route of such pharmaceutical compositions will be clear to the skilled person (reference is made to the latest edition of Remington's Pharmaceutical Sciences), and especially may be those listed above with regards to the pharmaceutical compositions of the invention.

In one embodiment, the kit of parts of the invention further comprises an additional therapeutic agent.

In the context of the present invention, the administration of the ENT inhibitor and the adenosine receptor antagonist may occur either simultaneously or timely staggered, either at the same site of administration or at different sites of administration, under similar or different dosage forms as further outlined below.

In one embodiment, the ENT inhibitor is administered prior to, concomitant with, or subsequent to administration of an adenosine receptor antagonist. To ensure that the separate mechanisms elicited by the ENT inhibitor and the adenosine receptor antagonist are not negatively influenced by each other, the adenosine receptor antagonist and the ENT inhibitor may be administered separated in time (in a time-staggered manner), i.e. sequentially, and/or are administered at different administration sites. This means that the adenosine receptor antagonist may be administrated e.g. prior, concurrent or subsequent to the ENT inhibitor, or vice versa. Alternatively or additionally, the adenosine receptor antagonist and the ENT inhibitor may be administered at different administration sites, or at the same administration site, preferably, when administered in a time staggered manner.

In one embodiment, the adenosine receptor antagonist is to be administered prior to and/or concomitantly with an ENT inhibitor. In one embodiment, the adenosine receptor antagonist is to be administered prior to the day or on the same day that the ENT inhibitor is administered. In another embodiment, the ENT inhibitor is to be administered prior to and/or concomitantly with an adenosine receptor antagonist. In one embodiment, the ENT inhibitor is to be administered prior to the day or on the same day that the adenosine receptor antagonist is administered. In one embodiment, the adenosine receptor antagonist is to be administered prior to and/or concomitantly with an ENT inhibitor and continuously thereafter. In another embodiment, the ENT inhibitor is to be administered prior to and/or concomitantly with an adenosine receptor antagonist and continuously thereafter.

Depending on the condition to be prevented or treated and the form of administration, the ENT inhibitor and the adenosine receptor antagonist may be administered as a single daily dose, divided over one or more daily doses.

It will be understood that the total daily usage of adenosine receptor antagonist and ENT inhibitor will be decided by the attending physician within the scope of sound medical judgment. The specific dose for any particular subject will depend upon a variety of factors such as the cancer to be treated; the age, body weight, general health, sex and diet of the patient; and like factors well-known in the medical arts.

Another object of this invention is the use of the combination as a medicament, i.e. for medical use. Thus, in one embodiment, the invention provides the use of the combination of the invention for the manufacturing of a medicament. Especially, the invention provides the use of the combined pharmaceutical composition of the invention or the kit of the invention for the manufacturing of a medicament.

Especially, the invention provides the combination, the combined pharmaceutical composition or the kit of parts of the invention, for use in the treatment and/or prevention of cancer. The invention further provides the use of the combination, combined pharmaceutical composition or kit of parts of the invention for the manufacture of a medicament for treating and/or preventing cancer. The invention further provides a method of treating of cancer, which comprises administering to a mammal species in need thereof a therapeutically effective amount of the combination, combined pharmaceutical composition or kit of parts of the invention.

Especially, the invention provides a method of treating cancer, comprising: administering, to a patient in need thereof, a combination of an adenosine receptor antagonist and an ENT inhibitor. The specific embodiments relative to the adenosine receptor antagonists and ENT inhibitors recited above also applies in the context of the methods of treatment of the invention.

The invention also provides for a method for delaying in patient the onset of cancer comprising the administration of a pharmaceutically effective amount of the combination, combined pharmaceutical composition or kit of parts of the invention to a patient in need thereof.

### EXAMPLES

The present invention will be better understood with reference to the following examples. These examples are intended to representative of specific embodiments of the invention, and are not intended as limiting the scope of the invention.

The following abbreviations are used:

THF: tetrahydrofuran;

DCM: dichloromethane;

DIEA: diisopropylethylamine;

N2: nitrogen gas;

min: minute;

hr: hour;

rt: retention time;

NMP: N-Methylpyrolidone;

DMF: Dimethylformamide;

Na2SO4: sodium sulfate;

prep-TLC: preparative Thin layer chromatography;

prep-HPLC: preparative High Pressure Liquid Chromatography;

HPLC: High Pressure Liquid Chromatography;

SiO2: silica gel;

MeOH: methanol;

FA: Formic acid

K2CO3: potassium carbonate;

HATU: hexafluorophosphate de (diméthylamino)-N,N-diméthyl(3H-[1,2,3]triazolo[4,5-b]pyridin-3-yloxy)méthaniminium;

BOP: benzotriazol-1-yloxytris(dimethylamino)phosphonium hexafluorophosphate;

DBU: 1,8-Diazabicyclo[5.4.0]undec-7-ene;

NaBH(OAC)3: sodium triacetoxyborohydride;

NaOH: sodium hydroxide.

MPLC: Medium pressure liquid chromatography

### I. Chemistry Examples

The MS data provided in the examples described below were obtained as follows:

LCMS were recorded using Agilent 6130 or 6130B multimode (ESI+APCI).

LCMS Methods:

Column: XBridge C8 (50×4.6 mm) 5 μm; Method: A: 0.1% TFA in H₂O, B: 0.1% TFA in ACN, Flow Rate: 2.0 mL/min.

The NMR data provided in the examples described below were obtained as followed:

1H-NMR: Bruker DPX 400 MHz. Abbreviations for multiplicities observed in NMR spectra are as follows: s (singlet), d (doublet), t (triplet), q (quadruplet), m (multiplet), br (broad).

HPLC Purity were evaluated with either of two methods:

Method XB0595TF; COLUMN: XBridge C8 (50×4.6) mm, 3.5 μm; Gradient of eluents from 0.1% TFA in H₂O to 0.1% TFA in ACN, Flow Rate: 2.0 mL/min.

Method AM9010A3; COLUMN: Phenomenex gemini NX-C18 (150×4.6), 3.0 μm; Gradient of eluents from 10 mM Ammonium acetate in water to ACN, Flow Rate: 1.0 mL/min.

The purification by preparative HPLC were done by the following three methods:

Condition A: prep-HPLC (column: Waters XBridge 150*25 mm*5 um; mobile phase: [water (10 mM NH4HCO3)-ACN]; B %: 70%-100%, 9 min).

Condition B: by prep-HPLC (column: 3_Phenomenex Luna C18 75*30 mm*3 um; mobile phase: [water (0.05% HCl)-ACN]; B %: 30%-50%, 7 min).

Condition C: prep-HPLC (column: Phenomenex Gemini NX-C18 (75*30 mm*3 um); mobile phase [water (10 mM NH4HCO3)-ACN]; B %: 66%-96%, 8 min).

Solvents, reagents and starting materials were purchased and used as received from commercial vendors unless otherwise specified.

### Example I1. Synthesis of Intermediate Compounds

Intermediate Compound 1:

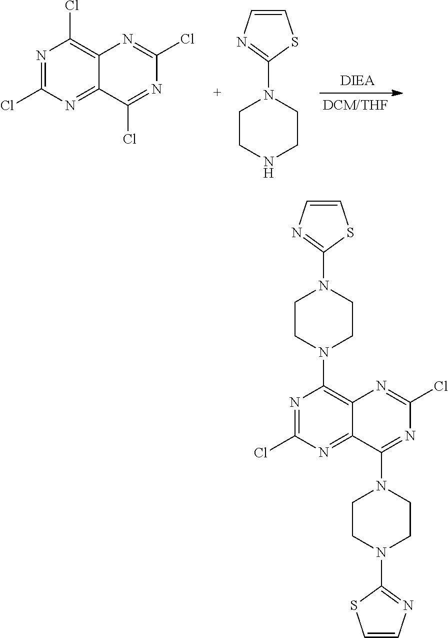 [SMILES: Clc1nc(Cl)c2nc(Cl)nc(Cl)c2n1 ; c1csc(N2CCNCC2)n1 ; Clc1nc(N2CCN(c3nccs3)CC2)c2nc(Cl)nc(N3CCN(c4nccs4)CC3)c2n1]

To a solution of perchloropyrimido[5,4-d]pyrimidine (100 mg, 370.50 μmol, 1 eq) in THF (1 mL) and DCM (0.5 mL) was added a solution of DIEA (120 mg, 928.48 μmol, 161.73 μl, 2.51 eq) and 2-(piperazin-1-yl)thiazole (126 mg, 744.47 μmol, 2.01 eq) in THF (0.5 mL) drop-wise at 0° C. over a period of 5 min under N2. During which the temperature was maintained 0-5° C. for 25 min. And the reaction mixture was stirred at 20° C. for 15 hr. The reaction mixture was concentrated under vacuum to give intermediate compound 1 (222 mg) as a yellow solid.

Intermediate Compound 2:

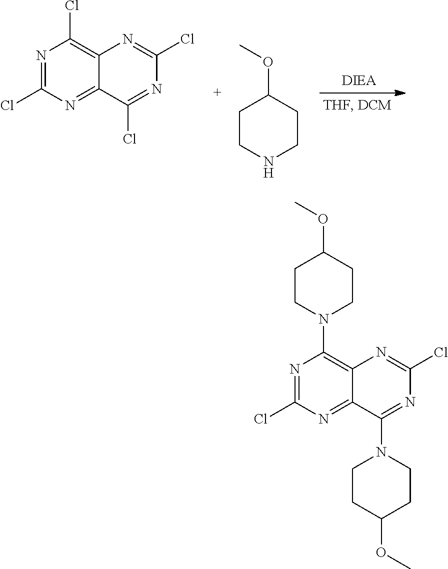

To a solution of perchloropyrimido[5,4-d]pyrimidine (100 mg, 370.50 μmol, 1 eq) in THF (1 mL) and DCM (0.5 mL) was added a solution of 4-methoxypiperidine (86 mg, 746.70 μmol, 2.02 eq) and DIEA (120 mg, 928.48 μmol, 161.73 μl, 2.51 eq) in THF (0.5 mL) dropwise at 0° C. over a period of 5 min under N2. During which the temperature was maintained 0-5° C. for 25 min. And the reaction mixture was stirred at 20° C. for 15 hr. The reaction mixture was concentrated to dryness under vacuum to give the intermediate compound 2 (218 mg) as a yellow solid, without further purification.

Intermediate Compound 3:

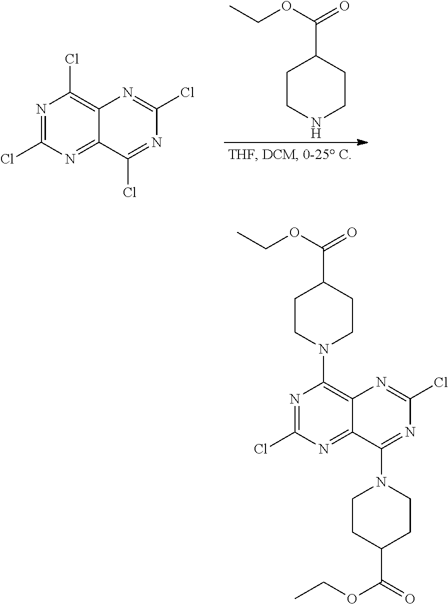 [SMILES: Clc1nc(Cl)c2nc(Cl)nc(Cl)c2n1 ; CCOC(=O)C1CCNCC1 ; CCOC(=O)C1CCN(c2nc(Cl)nc3c(N4CCC(C(=O)OCC)CC4)nc(Cl)nc23)CC1]

To a solution of perchloropyrimido[5,4-d]pyrimidine (100 mg, 370.50 μmol, 1 eq) in THF (1 mL) and DCM (0.5 mL) was added a solution of ethyl piperidine-4-carboxylate (122 mg, 776.03 μmol, 119.61 μl, 2.09 eq) and DIEA (120 mg, 928.48 μmol, 161.73 μl, 2.51 eq) in THF (0.5 mL) drop-wise at 0° C. over a period of 5 min under N2. The reaction mixture was stirred for 25 min at 0° C., and then stirred at 20° C. for 30 min. The reaction mixture was concentrated to dryness under vacuum to give the intermediate compound 3 (230 mg, crude) as a brown residual solid, used without further purification.

LCMS (ESI position ion) m/z: (M+H)+: 511.3 (calculated: 510.15)

Intermediate Compound 4:

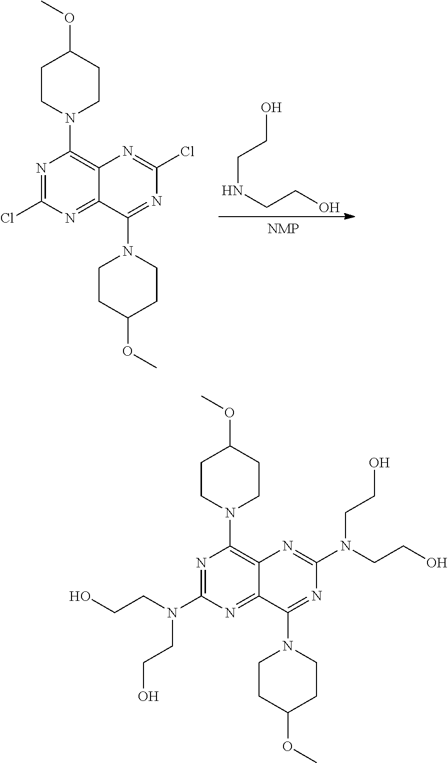 [SMILES: COC1CCN(c2nc(Cl)nc3c(N4CCC(OC)CC4)nc(Cl)nc23)CC1 ; OCCNCCO ; COC1CCN(c2nc(N(CCO)CCO)nc3c(N4CCC(OC)CC4)nc(N(CCO)CCO)nc23)CC1]

To a solution of intermediate compound 2 (200 mg, 468.03 μmol, 1 eq) and 2,2′-azanediylbis(ethan-1-ol) (443 mg, 4.21 mmol, 406.42 μl, 9 eq) in NMP (1 mL) was sealed and heated in microwave at 180° C. for 3 hr. The reaction mixture was diluted with water 50 mL and extracted with ethyl acetate 60 mL (20 mL*3). The combined organic layers were dried over Na2SO4, filtered and concentrated under reduced pressure to give a residue. The residue was purified by prep-TLC (SiO2, DCM/MeOH=15/1) to give the intermediate compound 4 (9.4 mg, 3.4% yield, 96.5% purity) as a yellow solid.

LCMS (ESI position ion) m/z: (M+H)+: 565.2 (calculated: 564.34)

Intermediate Compound 5:

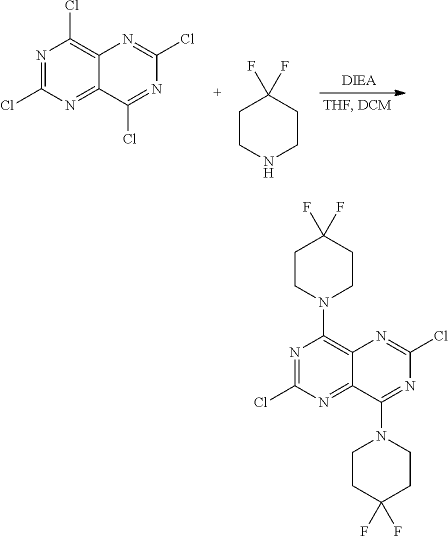 [SMILES: Clc1nc(Cl)c2nc(Cl)nc(Cl)c2n1 ; FC1(F)CCNCC1 ; FC1(F)CCN(c2nc(Cl)nc3c(N4CCC(F)(F)CC4)nc(Cl)nc23)CC1]

To a solution of perchloropyrimido[5,4-d]pyrimidine (200 mg, 741.01 μmol, 1 eq) in THF (2 mL) and DCM (1 mL) was added a solution of DIEA (240 mg, 1.86 mmol, 323.45 μl, 2.51 eq) and 4,4-difluoropiperidine (181 mg, 1.49 mmol, 2.02 eq) in THF (1 mL) dropwise at 0° C. in 5 min under N2, stirred at 0-5° C. for 25 min and at 20° C. for 30 min. The reaction mixture was concentrated under vacuum to give the intermediate compound 5 (496 mg) as a yellow solid and used without further purification.

Intermediate Compound 6:

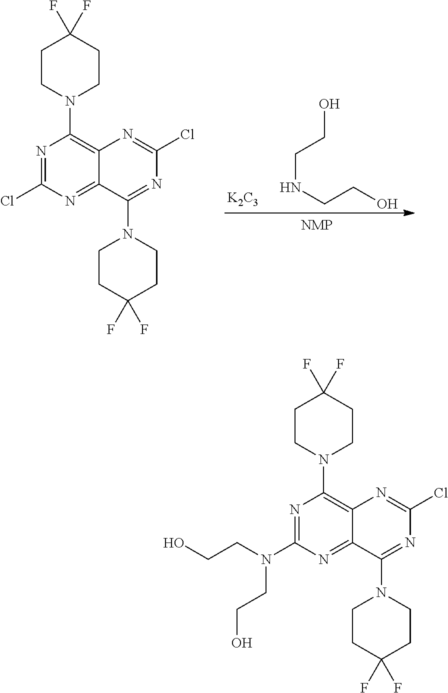

To a solution of intermediate compound 5 (200 mg, 455.34 μmol, 1 eq), 2,2′-azanediylbis(ethan-1-ol) (240 mg, 2.28 mmol, 220.18 μl, 5.01 eq) and K2C03 (158 mg, 1.14 mmol, 2.51 eq) in NMP (1 mL) was sealed and heated in microwave at 120° C. for 2 hr. The reaction mixture was filtered and the residue was purified by prep-HPLC (column: Waters Xbridge 150*25 mm*5 um; mobile phase: [water (10 mM NH4HCO3)-ACN]; B %: 41%-71%, 10 min) to give the intermediate compound 6 (50 mg, 21.6% yield, 100% purity) as a white solid.

Intermediate Compound 7:

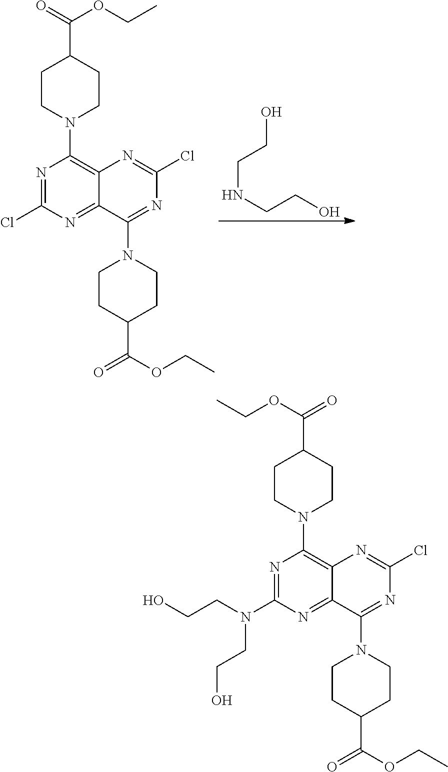

A mixture of intermediate compound 3 (100 mg, 195.54 μmol, 1 eq), DIEA (52 mg, 402.34 μmol, 70.08 μl, 2.06 eq) and 2,2′-azanediylbis(ethan-1-ol) (82 mg, 779.95 μmol, 75.23 μl, 3.99 eq) in NMP (0.5 mL) was stirred at 100° C. for 3 h under microwave. The reaction mixture was diluted with DCM (20 mL), and washed by water (20 mL, 3 times). The organic layer was dried with Na2SO4 and concentrated under reduced pressure to give the intermediate compound 7 (80 mg) as a yellow oil and used without further purification.

Intermediate Compound 8:

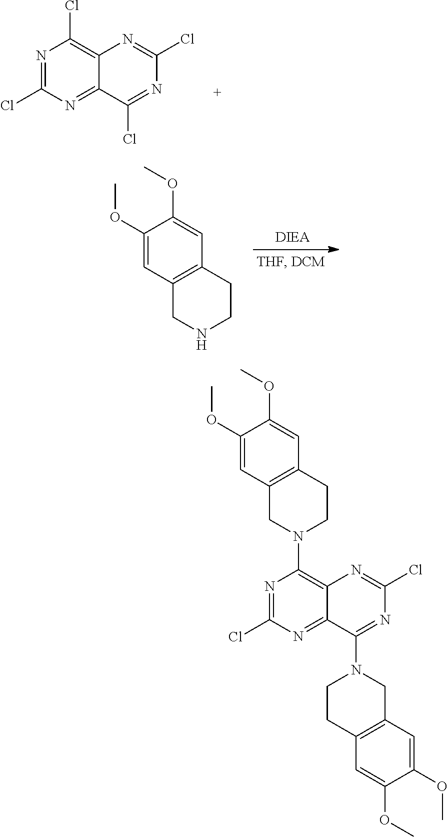

To a solution of perchloropyrimido[5,4-d]pyrimidine (100 mg, 370.50 μmol, 1 eq) in THF (1 mL) and DCM (0.5 mL) was added a solution of DIEA (120 mg, 928.48 μmol, 161.73 μl, 2.51 eq) and 6,7-dimethoxy-1,2,3,4-tetrahydroisoquinoline (150 mg, 776.23 μmol, 2.10 eq) in THF (0.5 mL) dropwise at 0° C. over a period of 5 min under N2. During which the temperature was maintained 0-5° C. for 25 min. And the reaction mixture was stirred at 20° C. for 15 hr. The reaction mixture was concentrated to dryness under vacuum to give the intermediate compound 8 (297 mg) as a yellow solid.

Intermediate Compound 9:

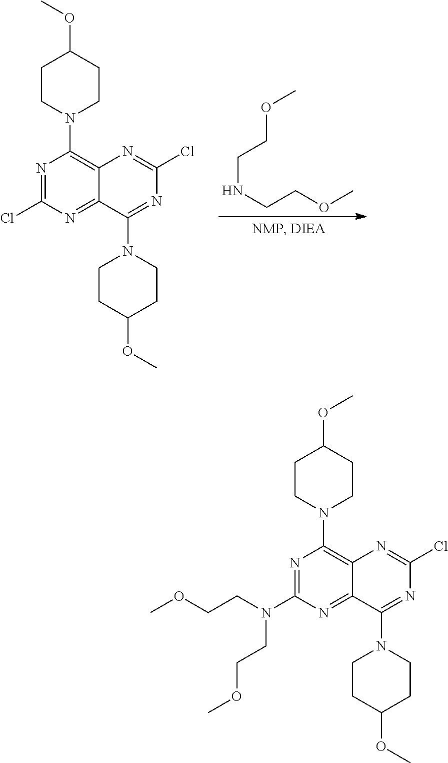

A solution of intermediate compound 2 (2 g, 4.68 mmol, 1 eq), bis(2-methoxyethyl)amine (2.56 g, 19.19 mmol, 2.83 mL, 4.10 eq) and DIEA (1.52 g, 11.75 mmol, 2.05 mL, 2.51 eq) in NMP (5 mL) was sealed and heated in microwave at 120° C. for 4 hr. The crude product was purified by re-crystallization from ethyl acetate (100 mL) at 25° C. to give the intermediate compound 9 (4.3 g) as a yellow oil.

Intermediate Compound 10:

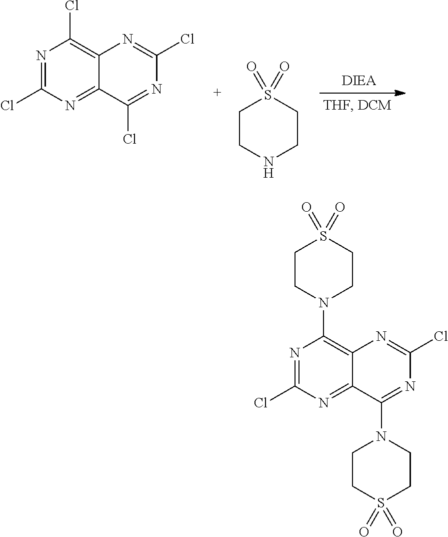 [SMILES: Clc1nc(Cl)c2nc(Cl)nc(Cl)c2n1 ; O=S1(=O)CCNCC1 ; O=S1(=O)CCN(c2nc(Cl)nc3c(N4CCS(=O)(=O)CC4)nc(Cl)nc23)CC1]

To a solution of perchloropyrimido[5,4-d]pyrimidine (200 mg, 741.01 μmol, 1 eq) in THF (2 mL) and DCM (1 mL) was added dropwise a solution of DIEA (240 mg, 1.86 mmol, 323.45 μl, 2.51 eq) and thiomorpholine 1,1-dioxide (202 mg, 1.49 mmol, 2.02 eq) in THF (1 mL) at 0° C. over a period of 5 min under N2. The reaction mixture was stirred at 0-5° C. for 25 min and at 20° C. for 15 hr. The reaction mixture was diluted with water (50 mL) and extracted with DCM (20 mL, 3 times). The combined organic layers were dried over Na2SO4, filtered and concentrated under reduced pressure. The residue was purified by re-crystallization from ethyl acetate (20 mL) at 20° C. to give the intermediate compound 10 (169 mg) as a yellow solid.

Intermediate Compound 11:

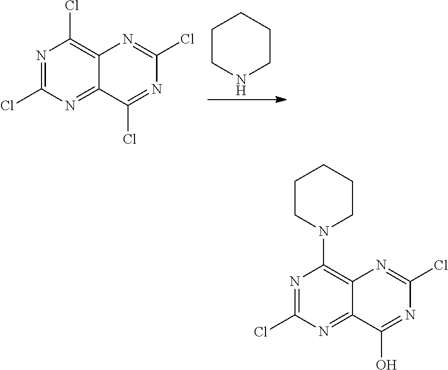

The intermediate compound 11 has been prepared by reaction the intermediate compound 21 with an excess of piperidine according to a methodology used and described for the intermediate compound 2.

Intermediate Compound 12:

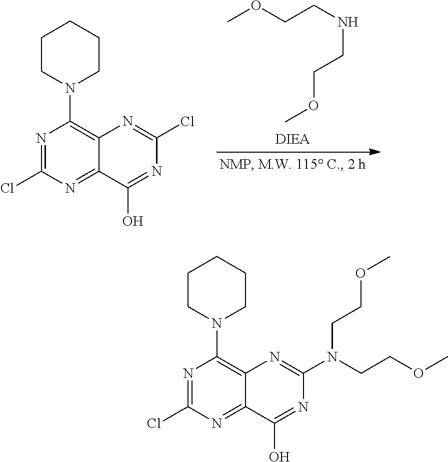

A solution of intermediate compound 11 (0.4 g, 1.33 mmol, 1 eq) and bis(2-methoxyethyl)amine (178 mg, 1.33 mmol, 1 eq), DIEA (513 mg, 2.2 eq) in NMP (1.3 mL) was stirred at 115° C. for 2 hr under microwave. The mixture of the two isomers (rt=0.795, 20% and rt=0.906, 80% UV) was purified by pre-HPLC (column: Phenomenex luna C18 150*40 mm*15 um; mobile phase: [water (0.1% TFA)-ACN]; B %: 38%-68%, 11 min) to give the intermediate compound 12 (rt=0.906, 175 mg, 32.8% yield, 99% purity) as a light yellow solid.

LCMS (ESI position ion) m/z: (M+H)+: 397.2 (calculated: 396.17).

Intermediate Compound 13:

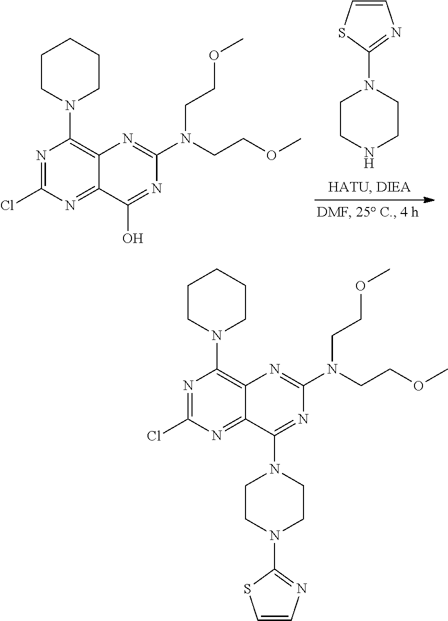

To a solution of intermediate compound 12 (100 mg, 249.45 μmol, 1 eq), DIEA (77 mg, 595.79 μmol, 103.77 μl, 2.39 eq) and 2-(piperazin-1-yl)thiazole (80 mg, 472.68 μmol, 8.99 μl, 1.89 eq) in DMF (1 mL), HATU (188 mg, 494.44 μmol, 1.98 eq) was added, and the mixture was stirred at 25° C. for 4 hr. The mixture was poured into water (20 mL), extracted with DCM (20 mL). The organic layer was concentrated to give the intermediate compound 13 (110 mg) as a red oil, used without further purification.

LCMS (ESI position ion) m/z: (M+H)+: 548.3 (calculated 574.22)

Intermediate Compound 14:

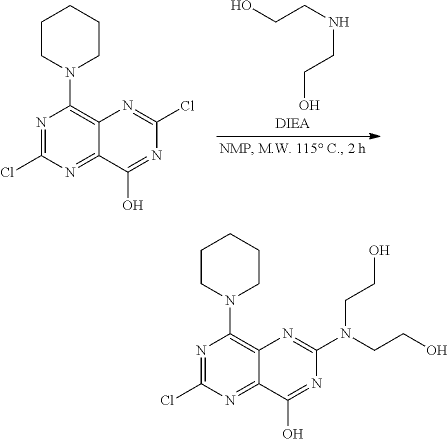 [SMILES: Oc1nc(Cl)nc2c(N3CCCCC3)nc(Cl)nc12 ; OCCNCCO ; OCCN(CCO)c1nc(O)c2nc(Cl)nc(N3CCCCC3)c2n1]

A mixture intermediate compound 11 (0.8 g, 2.67 mmol, 1 eq) and 2,2′-azanediylbis(ethan-1-ol) (281 mg, 2.67 mmol, 1 eq), DIEA (759.16 mg, 5.87 mmol, 1.02 mL, 2.2 eq) in NMP (2.5 mL) was stirred at 115° C. for 2 hr under microwave. The mixture of the two isomers: (rt=0.749 min, 20% and rt=0.870 min, 80% UV) was purified by pre-HPLC (column: Phenomenex luna C18 150*40 mm*15 um; mobile phase: [water (0.1% TFA)-ACN]; B %: 15%-45%, 11 min) to give the intermediate compound 14 (0.2 g, 20.3% yield, 100% purity) as a light yellow solid.

LCMS (ESI position ion) m/z: (M+H)+: 369.2 (calculated: 368.14)

Intermediate Compound 15:

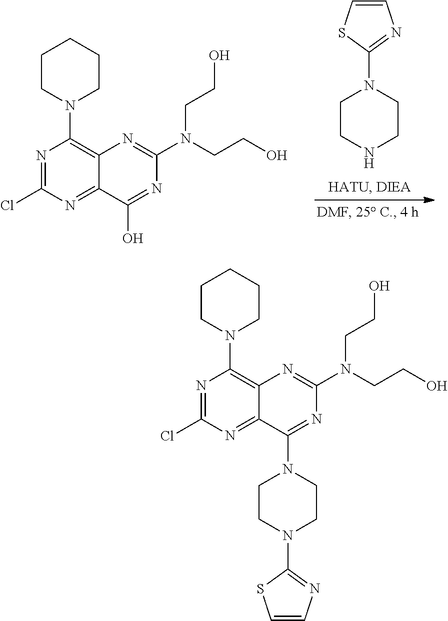 [SMILES: OCCN(CCO)c1nc(O)c2nc(Cl)nc(N3CCCCC3)c2n1 ; c1csc(N2CCNCC2)n1 ; OCCN(CCO)c1nc(N2CCN(c3nccs3)CC2)c2nc(Cl)nc(N3CCCCC3)c2n1]

To a mixture of intermediate compound 14 (200 mg, 542.27 μmol, 1 eq), DIEA (141 mg, 1.09 mmol, 190.03 μl, 2.01 eq) and 2-(piperazin-1-yl)thiazole (137 mg, 809.47 μmol, 8.99 μl, 1.49 eq) in DMF (2 mL), HATU (381 mg, 1.00 mmol, 1.85 eq) was added, the mixture was stirred at 25° C. for 4 hr. The mixture was filtered and then purified by pre-HPLC (column: Phenomenex luna C18 150*40 mm*15 um; mobilephase: [water (0.1% TFA)-ACN]; B %: 20%-50%, 11 min) to give the intermediate 0compound 15 (0.24 g, 57.9% yield, 83% purity, TFA) as a yellow solid.

LCMS (ESI position ion) m/z: (M+H)+: 520.3 (calculated: 519.19)

Intermediate Compound 16:

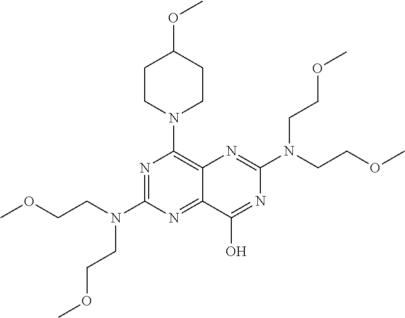

The intermediate compound 16 has been prepared from intermediate compound 21, 4-methoxypiperidine and bis(2-methoxyethyl)amine by a methodology used and described for the intermediate compound 23.

Intermediate Compound 17:

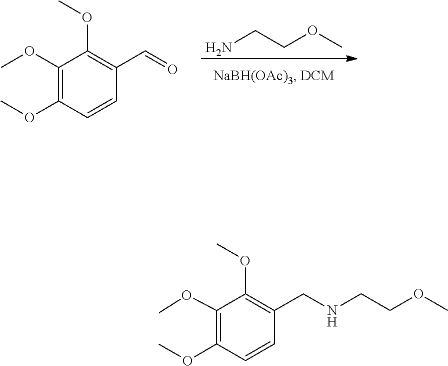

To a solution of 2,3,4-trimethoxybenzaldehyde (8.0 g, 40.77 mmol, 1 eq) and 2-methoxyethan-1-amine (3.68 g, 48.93 mmol, 4.25 mL, 1.2 eq) in DCM (100.0 mL) was added AcOH (2.45 g, 40.77 mmol, 2.33 mL, 1 eq) in one portion. This reaction mixture was stirred at 20° C. for 1 hr. Then NaBH(OAc)₃ (12.96 g, 61.16 mmol, 1.5 eq) was added into this mixture slowly at 0° C. After addition, this reaction by addition of an aqueous solution of was warmed to 20° C. and was stirred for 3 hr. This reaction mixture was quenched by addition of an aqueous solution of NaOH (1 M, 80.0 mL) and was extracted by DCM (50.0 mL*3). The combined organic layers were dried over Na2SO4 and concentrated The residue was purified by reversed-phase HPLC (0.1% NH3.H2O) to give the intermediate compound 17 (6.8 g, 63.8% yield, 98% purity) as yellow oil.

LCMS (ESI position ion) m/z: (M+H)+: 256.3 (calculated: 255.15) ¹H NMR (400 MHz, CHLOROFORM-d) δ=6.95 (d, J=8.4 Hz, 1H), 6.62 (d, J=8.5 Hz, 1H), 3.91 (s, 3H), 3.86 (s, 3H), 3.84 (s, 3H), 3.75 (s, 2H), 3.55-3.47 (m, 2H), 3.34 (s, 3H), 2.78 (t, J=5.3 Hz, 2H).

Intermediate Compound 18:

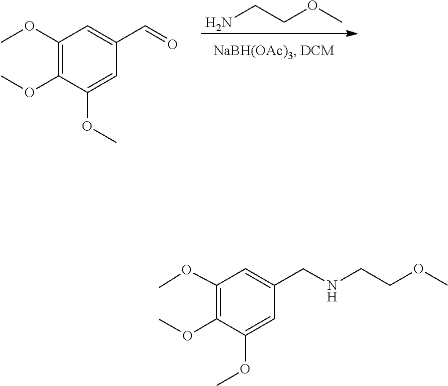

To a solution of 3,4,5-trimethoxybenzaldehyde (8.0 g, 40.77 mmol, 1 eq) and 2-methoxyethan-1-amine (3.68 g, 48.93 mmol, 4.25 mL, 1.2 eq) in DCM (100.0 mL) was added AcOH (2.45 g, 40.77 mmol, 2.33 mL, 1 eq) in one portion. This reaction mixture was stirred at 20° C. for 1 hr. Then NaBH(OAc)₃ (12.96 g, 61.16 mmol, 1.5 eq) was added into this mixture slowly at 0° C. After addition, this reaction mixture was warmed to 20° C. and was stirred for 3 hr. The reaction mixture was quenched by addition of an aqueous solution of NaOH (1 M, 80.0 mL) and was extracted by DCM (50.0 mL, 3 times). The combined organic layers were dried over Na2SO4 and concentrated. The residue was purified by reversed-phase HPLC (0.1% NH3.H2O) to give the intermediate compound 18 (6.3 g, 59.5% yield, 98.4% purity) as a yellow oil. LCMS (ESI position ion) m/z: (M+H)+: 256.3 (calculated: 255.15). ¹H NMR (400 MHz, CHLOROFORM-d) δ=6.57 (s, 2H), 3.86 (s, 6H), 3.82 (s, 3H), 3.75 (s, 2H), 3.55-3.50 (m, 2H), 3.36 (s, 3H), 2.81 (t, J=5.1 Hz, 2H).

Intermediate Compound 19:

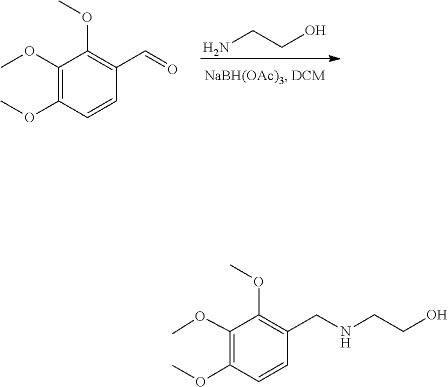

To a solution of 2,3,4-trimethoxybenzaldehyde (8.0 g, 40.77 mmol, 1 eq) and 2-aminoethan-1-ol (2.99 g, 48.92 mmol, 2.96 mL, 1.2 eq) in DCM (100.0 mL) was added AcOH (2.45 g, 40.77 mmol, 2.33 mL, 1 eq) in one portion. This reaction mixture was stirred at 20° C. for 1 hr. Then NaBH(OAc)₃ (12.96 g, 61.16 mmol, 1.5 eq) was added into this mixture slowly at 0° C. After addition, this reaction mixture was warmed to 20° C. and was stirred for 3 hr. The reaction mixture was quenched by addition of an aqueous solution of NaOH (1 M, 80.0 mL) and was extracted by DCM (50.0 mL, 3 times). The combined organic layers were dried over Na2SO4 and concentrated. The residue was purified by reversed-phase HPLC (0.1% NH3.H2O) to give the intermediate compound 19 (4.2 g, 39.18% yield, 91.8% purity) as a yellow oil. LCMS (ESI position ion) m/z: (M+H)+: 242.3 (calculated: 241.13). ¹H NMR (400 MHz, CHLOROFORM-d) δ=6.92 (d, J=8.4 Hz, 1H), 6.62 (d, J=8.4 Hz, 1H), 3.91 (s, 3H), 3.86 (s, 3H), 3.84 (s, 3H), 3.72 (s, 2H), 3.65-3.60 (m, 2H), 2.77-2.72 (m, 2H).

Intermediate Compound 20:

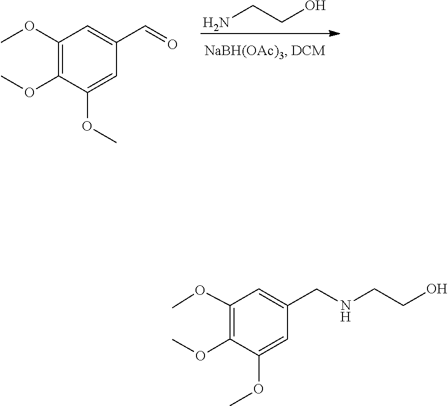

To a solution of 3,4,5-trimethoxybenzaldehyde (8.0 g, 40.77 mmol, 1 eq) and 2-aminoethan-1-ol (2.99 g, 48.92 mmol, 2.96 mL, 1.2 eq) in DCM (100.0 mL) was added AcOH (2.45 g, 40.77 mmol, 2.33 mL, 1 eq) in one portion. This reaction mixture was stirred at 20° C. for 1 hr. Then NaBH(OAc)₃ (12.96 g, 61.16 mmol, 1.5 eq) was added into this mixture slowly at 0° C. After addition, this reaction mixture was warmed to 20° C. and was stirred for 3 hr. The reaction mixture was quenched by addition of an aqueous solution of NaOH (1 M, 80.0 mL) and was extracted by DCM (50.0 mL, 3 times). The combined organic layers were dried over Na2SO4 and concentrated. The residue was purified by reversed-phase HPLC (0.1% NH3.H2O) to give the intermediate compound 20 (3.7 g, 36.97% yield, 98.3% purity) as a brown oil. LCMS (ESI position ion) m/z: (M+H)+: 242.3 (calculated: 241.13). ¹H NMR (400 MHz, CHLOROFORM-d) δ=6.55 (s, 2H), 3.85 (s, 6H), 3.82 (s, 3H), 3.73 (s, 2H), 3.67 (t, J=5.1 Hz, 2H), 2.79 (t, J=5.0 Hz, 2H).

Intermediate Compound 21:

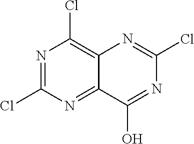 [SMILES: Oc1nc(Cl)nc2c(Cl)nc(Cl)nc12]

The intermediate compound 21 has been prepared according to the procedure described in WO2017003822A1 (compound 68 page 121).

Intermediate Compound 22:

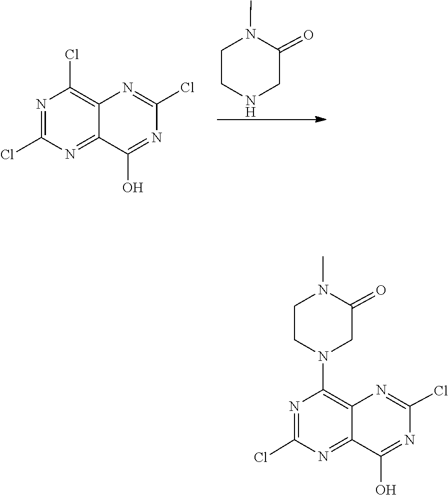

To a mixture of intermediate compound 21 (500 mg, 1.99 mmol, 1 eq) in THF (2.5 mL) was added DIEA (515 mg, 3.98 mmol, 694.07 μl, 2 eq) and 1-methylpiperazin-2-one (227 mg, 1.99 mmol, 39.43 μl, 1 eq) in one portion at 0° C. for 30 min under N2. The mixture was stirred at 10° C. for 14.5 hr. The crude product was triturated with water (30 mL) at 16° C. for 20 min. The mixture was filtered and the filter cake was washed with 30 mL of citric acid at 16° C. for 20 min. The mixture was filtered and the filter cake was washed with water (30 mL) at 16° C. for 20 min, dried in vacuum to give the intermediate compound 22 (200 mg) as a white solid.

Intermediate Compound 23:

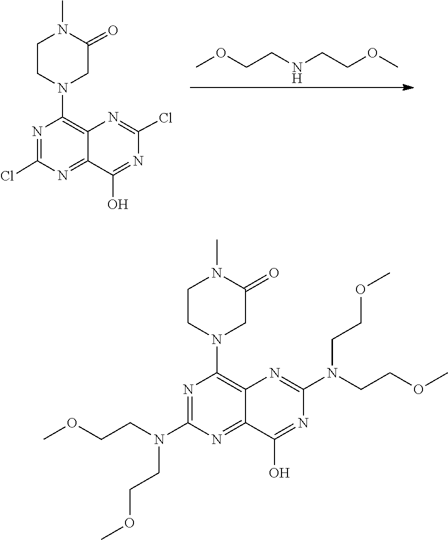

To a solution of intermediate compound 22 (200 mg, 607.64 μmol, 1 eq), bis(2-methoxyethyl)amine (728 mg, 5.47 mmol, 807.10 μl, 9 eq) and DIEA (200 mg, 1.55 mmol, 269.54 μl, 2.55 eq) in NMP (2 mL) was sealed and heated in microwave at 180° C. for 2 hr. The reaction mixture was diluted with water 50 mL and extracted with DCM (20 mL, 3 times). The combined organic layers were dried over Na2SO4, filtered and concentrated under reduced pressure. The residue was purified by prep-HPLC (column: Waters XBridge 150*25 mm*5 um; mobile phase: [water (10 mM NH4HCO3)-ACN]; B %: 15%-45%, 9 min) to give the intermediate compound 23 (445 mg) as a yellow solid.

Intermediate Compound 24:

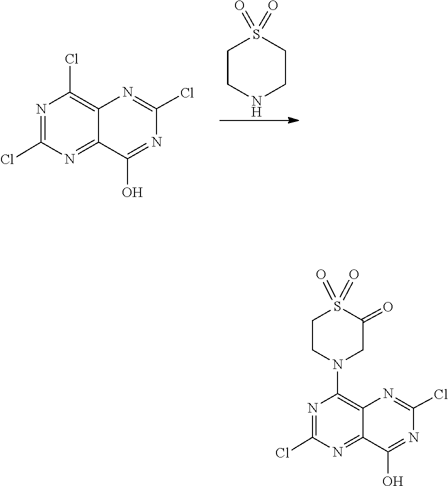

To a mixture of intermediate compound 21 (500 mg, 1.99 mmol, 1 eq) in THF (2.5 mL) was added DIEA (515 mg, 3.98 mmol, 694.07 μl, 2 eq) and thiomorpholine 1,1-dioxide (270 mg, 2.00 mmol, 39.43 μl, 1 eq) in one portion at 0° C. for 30 min under N2. The mixture was stirred at 10° C. for 14.5 hr. The crude product was triturated with water (30 mL) at 16° C. for 20 min. The mixture was filtered and the filter cake was washed with 30 mL of citric acid at 16° C. for 20 min. The mixture was filtered and the filter cake was washed with water (30 mL) at 16° C. for 20 min, dried in vacuum to give the intermediate compound 24 (212 mg) as a yellow solid.

Intermediate Compound 25:

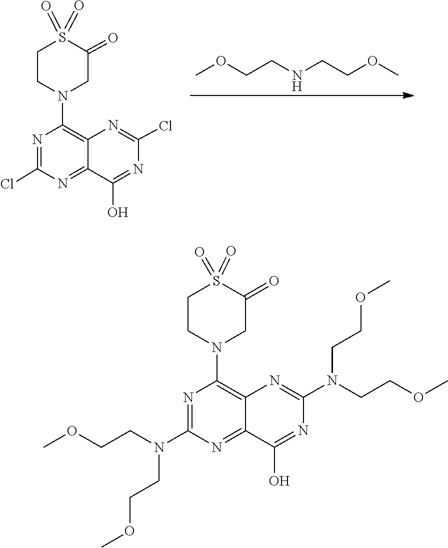

To a solution of intermediate compound 24 (200 mg, 571.13 μmol, 1 eq), DIEA (188 mg, 1.45 mmol, 253.37 μl, 2.55 eq) and bis(2-methoxyethyl)amine (685 mg, 5.14 mmol, 759.42 μl, 9.01 eq) in NMP (2 mL) was sealed and heated in microwave at 180° C. for 2 hr. The reaction mixture was diluted with water 50 mL and extracted with DCM 60 mL (20 mL, 3 times). The combined organic layers were dried over Na2SO4, filtered and concentrated under reduced pressure. The residue was purified by prep-HPLC (column: Waters XBridge 150*25 mm*5 um; mobile phase: [water (10 mM NH4HCO3)-ACN]; B %: 17%-47%, 9 min) to give the intermediate compound 25 (100 mg) as a yellow solid.

Intermediate Compound 26:

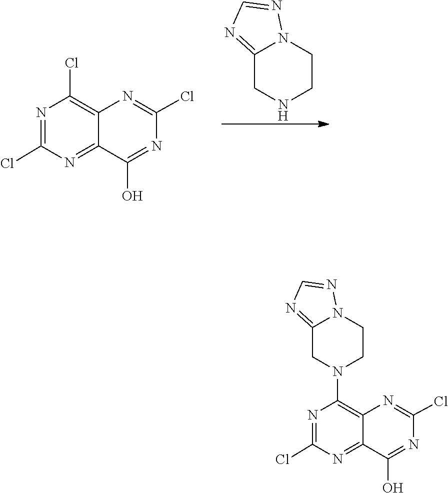 [SMILES: Oc1nc(Cl)nc2c(Cl)nc(Cl)nc12 ; c1nc2n(n1)CCNC2 ; Oc1nc(Cl)nc2c(N3CCn4ncnc4C3)nc(Cl)nc12]

To a mixture of intermediate compound 21 (500 mg, 1.99 mmol, 1 eq) in THF (2.5 mL) was added DIEA (515 mg, 3.98 mmol, 694.07 μl, 2 eq) and 5,6,7,8-tetrahydro-[1,2,4]triazolo[1,5-a]pyrazine (247 mg, 1.99 mmol, 39.43 μl, 1.00 eq) in one portion at 0° C. for 30 min under N2. The mixture was stirred at 10° C. for 14.5 hr. The crude product was triturated with water (30 mL) at 16° C. for 20 min. The mixture was filtered and the filter cake was washed with 30 mL of citric acid at 16° C. for 20 min. The mixture was filtered and the filter cake was washed with water (30 mL) at 16° C. for 20 min, dried in vacuum to give the intermediate compound 26 (230 mg) as a yellow solid.

Intermediate Compound 27:

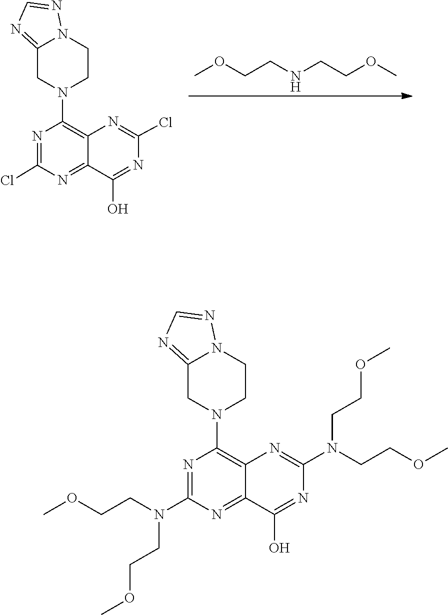

To a mixture of intermediate compound 26 (200 mg, 589.73 μmol, 1 eq), bis(2-methoxyethyl)amine (708 mg, 5.32 mmol, 784.92 μl, 9.01 eq) and DIEA (190 mg, 1.47 mmol, 256.06 μl, 2.49 eq) in NMP (2 mL) was sealed and heated in microwave at 180° C. for 2 hr. The reaction mixture was diluted with water 50 mL and extracted with DCM 60 mL (20 mL, 3 times). The combined organic layers were dried over Na2SO4, filtered and concentrated under reduced pressure. The residue was purified by prep-HPLC (column: Waters XBridge 150*25 mm*5 um; mobile phase: [water (10 mM NH4HCO3)-ACN]; B %: 12%-42%, 9 min) to give the intermediate compound 27 (730 mg) as a yellow solid.

Intermediate Compound 28:

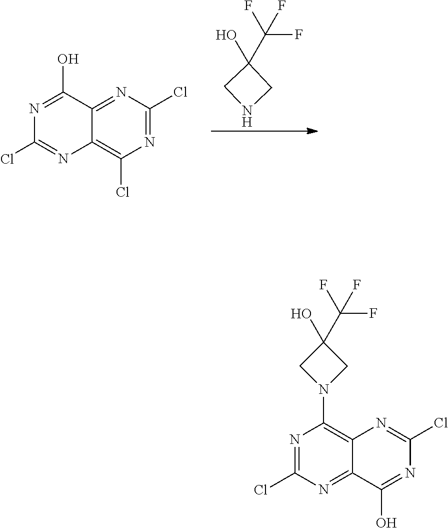

To a mixture of intermediate compound 21 (200 mg, 795.36 μmol, 1 eq) in THF (1 mL) was added DIEA (206 mg, 1.59 mmol, 277.63 μl, 2 eq) and 3-(trifluoromethyl)azetidin-3-ol (141 mg, 794.13 μmol, 39.43 μl, 9.98e-1 eq, HCl) in one portion at 0° C. for 30 min under N2. The mixture was stirred at 10° C. for 14.5 hr. The crude product was triturated with water (30 mL) at 16° C. for 20 min. The mixture was filtered and the filter cake was washed with 30 mL of citric acid at 16° C. for 20 min. The mixture was filtered and the filter cake was washed with water (30 mL) at 16° C. for 20 min, dried in vacuum to give the intermediate compound 28 (113 mg) as a yellow solid.

Intermediate Compound 29:

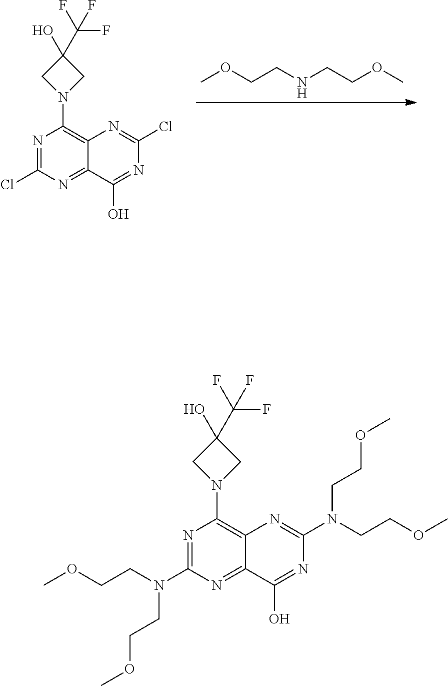

To a mixture of intermediate compound 28 (100 mg, 280.83 μmol, 1 eq), bis(2-methoxyethyl)amine (336 mg, 2.52 mmol, 372.51 μl, 8.98 eq) and DIEA (91 mg, 704.10 μmol, 122.64 μl, 2.51 eq) in NMP (1 mL) was sealed and heated in microwave at 180° C. for 2 hr. The reaction mixture was diluted with water 50 mL and extracted with DCM 60 mL (20 mL, 3 times). The combined organic layers were dried over Na2SO4, filtered and concentrated under reduced pressure. The residue was purified by prep-HPLC (column: Waters XBridge 150*25 mm*5 um; mobile phase: [water (10 mM NH4HCO3)-ACN]; B %: 22%-52%, 9 min) to give the intermediate compound 29 (90 mg) as a yellow solid.

Intermediate Compound 30:

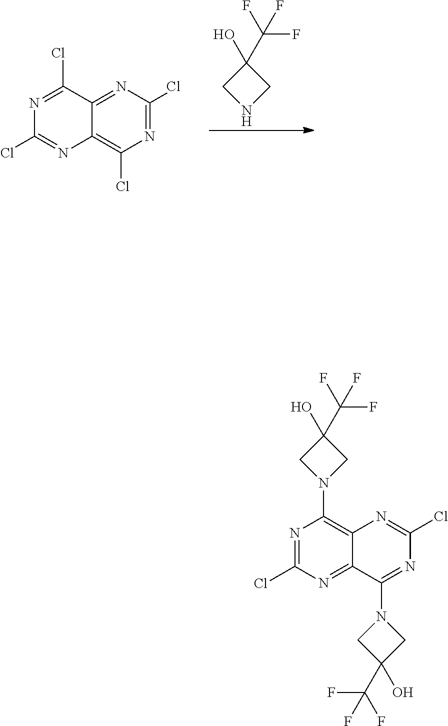

To a solution of perchloropyrimido[5,4-d]pyrimidine (100 mg, 370.50 μmol, 1 eq) in THF (1 mL) and DCM (0.5 mL) was added DIEA (216 mg, 1.67 mmol, 291.11 μl, 4.51 eq) and 3-(trifluoromethyl)azetidin-3-ol (131 mg, 737.81 μmol, 1.99 eq, HCl) in THF (0.5 mL) at 0° C. for 30 min under N2. The mixture was stirred at 16° C. for 14.5 hr. The reaction mixture was concentrated to dryness under vacuum. The yellow solid obtained was diluted with water 20 ml and acidified with citric acid to pH=7 and then stirred at 16° C. for 1 hr. The mixture was filtered, washed with 60 mL of MeCN, dried in vacuum to give the intermediate compound 30 (183 mg) as a yellow solid

Intermediate Compound 31:

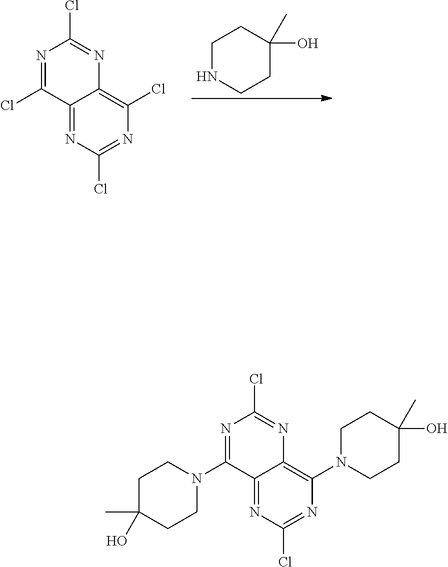

To a solution of perchloropyrimido[5,4-d]pyrimidine (200 mg, 741.01 μmol, 1 eq) in THF (2 mL) and DCM (1 mL) was added a solution of DIEA (240 mg, 1.86 mmol, 323.45 μL, 2.51 eq) and 4-methylpiperidin-4-ol (171 mg, 1.48 mmol, 2 eq) in THF (1 mL) dropwise at 0° C. over a period of 5 min under nitrogen, and stirred at 0-5° C. for 25 min, then the reaction mixture was stirred at 16° C. for 14.5 hrs. The reaction mixture was concentrated to dryness under vacuum to give a yellow solid. The crude product was diluted with water 50 ml. The aqueous phase was acidified with citric acid to pH=7 and stirred at 16° C. for 30 min. The mixture was filtered and the filter cake was washed with 60 mL of DCM, dried in vacuum to give the Intermediate compound 31 (373 mg, crude) as a yellow solid. LCMS (ESI position ion) m/z: (M+H)+: 427.1 (calculated: 427.1).

Intermediate Compound 32:

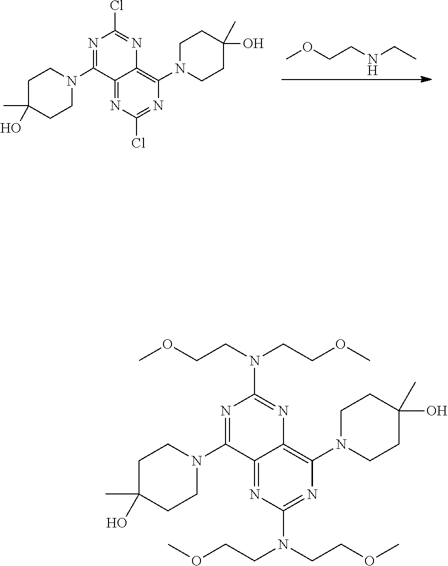

To a solution of Intermediate compound 31 (100 mg, 234.01 μmol, 1 eq), bis(2-methoxyethyl)amine (281 mg, 2.11 mmol, 311.53 μL, 9.02 eq) and DIEA (76 mg, 588.04 μmol, 102.43 μL, 2.51 eq) in NMP (1 mL) was sealed and heated in microwave at 180° C. for 2 hrs. The reaction mixture was sealed and heated in microwave at 190° C. for 2 hrs. The reaction mixture was diluted with water 30 mL. The aqueous phase was acidified with citric acid to pH=7 and stirred at 16° C. for 30 min and extracted with EtOAc (20 mL, 3 times). The combined organic layers were dried over Na₂SO₄, filtered and concentrated under reduced pressure. The residue was purified by prep-HPLC (column: Phenomenex luna C18 150*25 mm*10 um; mobile phase: [water (0.05% HCl)-ACN]; B %: 22%-52%, 10 min) to give the Intermediate compound 32 (90 mg, 142.08 μmol, 61% yield) as a yellow oil. LCMS (ESI position ion) m/z: (M+H)+: 621.3 (calculated: 621.4).

Intermediate Compound 33:

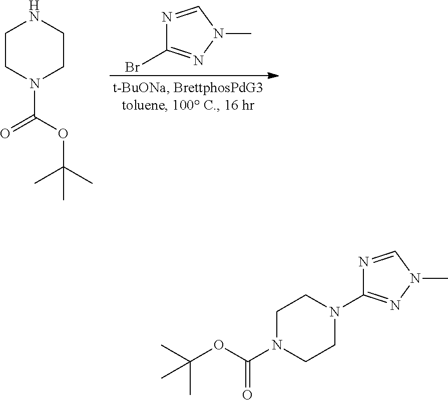

The reaction was done 2 times in parallel.

To a mixture of tert-butyl piperazine-1-carboxylate (1.17 g, 6.3 mmol) and 3-bromo-1-methyl-1H-1,2,4-triazole (0.85 g, 5.3 mmol) in toluene (20 mL) was added sodium tert-butoxide (1.01 g, 10.5 mmol) and BrettPhos-Pd-G₃ (475.7 mg, 524.7 μmol) at 25° C. After purged and degassed with nitrogen for 10 min, the mixture was stirred at 100° C. for 16 hr under nitrogen. After cooling to room temperature, the two reaction mixtures were combined and then filtered over a pad of celite. The filter cake was washed with DCM (50 mL, 2 times). The combined organic phases were concentrated under reduced pressure. The residue was purified by column on silica gel (eluted with petroleum ether/ethyl acetate=5/1 to 0/1) to give Intermediate compound 33 (2.3 g, yield 61%) as brown oil. LCMS (ESI position ion) m/z: (M+H)+: 268.1 (calculated: 268.2). ¹H NMR (ET28588-1008-P1N1, CDCl₃-d 400 MHz) δ ppm 6.84 (s, 1H), 3.06 (s, 3H), 2.99-2.96 (m, 4H), 2.41-2.38 (m, 4H), 1.03 (s, 9H)

Intermediate Compound 34:

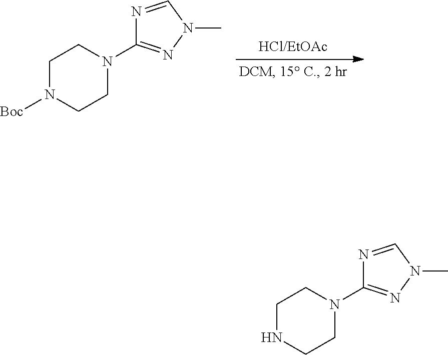 [SMILES: Cn1cnc(N2CCN(C(=O)OC(C)(C)C)CC2)n1 ; Cn1cnc(N2CCNCC2)n1]

To a solution of Intermediate compound 33 (2.3 g, 8.6 mmol) in DCM (30 mL) was added hydrogen chloride in ethyl acetate (20 mL, 4 M) at 15° C. The reaction mixture was stirred at 15° C. for 2 hr. The solvent was removed under reduced pressure and then concentrated in high vacuum to give Intermediate compound 34 (2.27 g, crude) as white solid.

LCMS (ESI position ion) m/z: (M+H)+: 168.0 (calculated: 168.1).

Intermediate Compound 35:

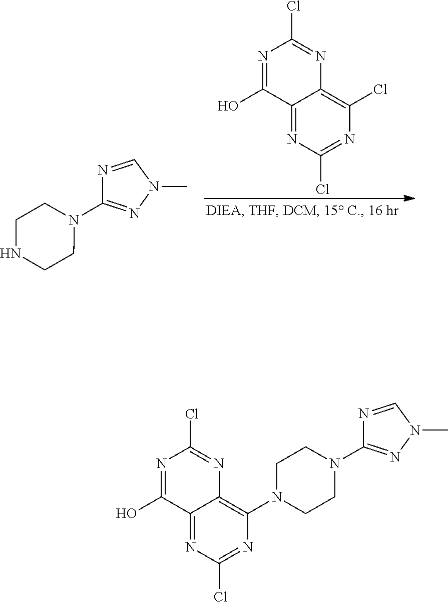 [SMILES: Cn1cnc(N2CCNCC2)n1 ; Oc1nc(Cl)nc2c(Cl)nc(Cl)nc12 ; Cn1cnc(N2CCN(c3nc(Cl)nc4c(O)nc(Cl)nc34)CC2)n1]

To a solution of Intermediate compound 21 (1 g, 3.98 mmol,) in tetrahydrofuran (10 mL) was added a mixture of Intermediate compound 34 (1.1 g, 3.98 mmol) and DIEA (1.54 g, 11.9 mmol) in DCM (5 mL) at 0° C. The reaction mixture was stirred at 15° C. for 16 hr. The reaction mixture of crude product was purified by reverse MPLC (eluted with methanol/H₂O=40%) to give Intermediate compound 35 (200 mg, yield 9%) as brown solid. LCMS (ESI position ion) m/z: (M+H)+: 382.1 (calculated: 382.1). ¹H NMR (ET28588-1021-P1N, DMSO-d₆ 400 MHz) δ ppm 8.26-8.21 (m, 1H), 4.37-4.09 (m, 4H), 3.72-3.66 (m, 3H), 3.49-3.34 (m, 4H)

Intermediate Compound 36:

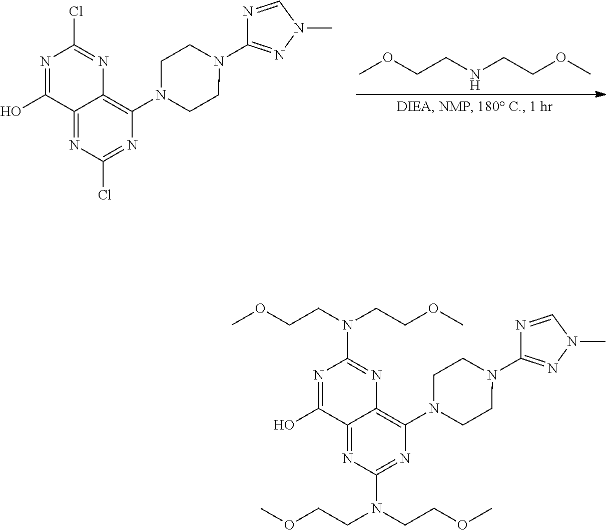

To a mixture of Intermediate compound 35 (150 mg, 392.5 μmol) in NMP (1.5 mL) was added DIEA (101.44 mg, 784.9 μmol) and bis(2-methoxyethyl)amine (522.71 mg, 3.9 mmol) at 25° C. Then the reaction mixture was stirred at 180° C. for 1 hr under microwave. The reaction mixture was poured into water (20 mL) and then extracted with chloroform (10 mL, 2 times). The combined organic phase was washed with brine (20 mL), dried over anhydrous Na₂SO₄, filtered and concentrated under reduced pressure. The residue was purified by column on silica gel (eluted with ethyl acetate/methanol=100/0 to 5/1) to give Intermediate compound 36 (150 mg, yield 66%) as brown oil. LCMS (ESI position ion) m/z: (M+H)+: 576.4 (calculated: 576.3)

Intermediate Compound 37:

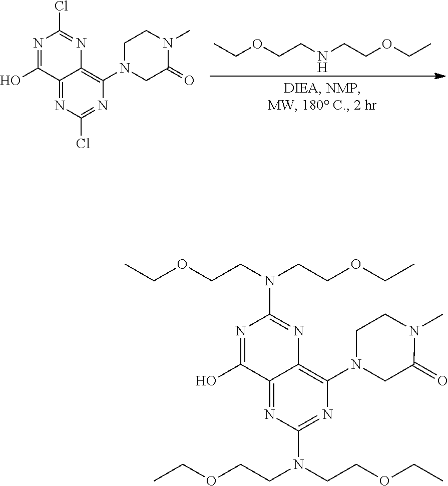

To a solution of Intermediate compound 22 (200 mg, 607.64 μmol) and bis(2-ethoxyethyl)amine (489.88 mg, 3.04 mmol) in NMP (2 mL) was added DIEA (196.33 mg, 1.52 mmol, 264.60 μL) at 25° C. The reaction mixture was stirred at 150° C. for 1 hr under microwave. The reaction mixture was partitioned between water (10 mL) and extracted by ethyl acetate (5 mL, 3 times). The organic phase was combined, washed with brine (10 mL), dried over Na2SO4, filtered and concentrated under reduced pressure. The residue was purified by prep-HPLC (Method B) to give the Intermediate compound 37 (180 mg, yield 51%) as yellow oil. LCMS (ESI position ion) m/z: (M+H)+: 579.4 (calculated: 579.3)

Intermediate Compound 38:

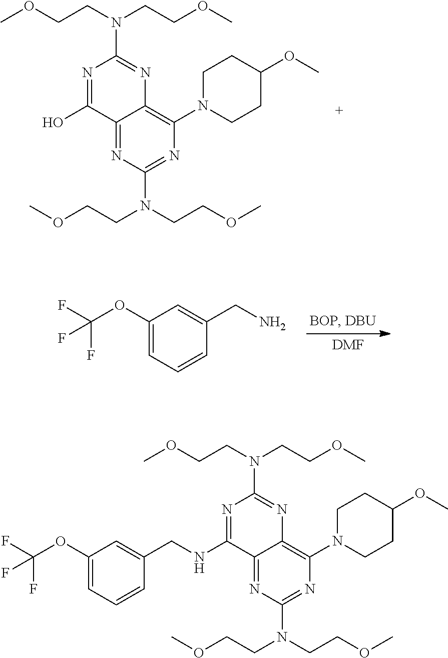

To a solution of Intermediate compound 16 (60 mg, 114.59 μmol, 1 eq), DBU (25.99 mg, 170.73 μmol, 25.73 μL, 1.49 eq) and BOP (65.88 mg, 148.96 μmol, 1.3 eq) in DMF (1.5 mL) was stirred at 0° C. for 20 min. The mixture was added (3-(trifluoromethoxy)phenyl) methanamine (65.71 mg, 343.76 μmol, 3 eq). The mixture was stirred at 0-16° C. for 15 hrs. The reaction mixture was diluted by DCM (20 mL) and the organic solution was washed with brine (20 mL, 3 times). The combined organic layers were dried over anhydrous Na2SO4 and concentrated under vacuum to give the Intermediate compound 38 (70 mg, 88% yield) as brown oil. LCMS (ESI position ion) m/z: (M+H)+: 697.4 (calculated: 697.3)

Intermediate Compound 39:

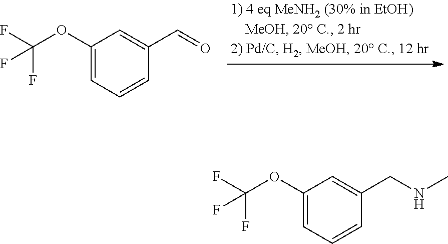

To a solution of 3-(trifluoromethoxy)benzaldehyde (100 mg, 525.9 μmol) in MeOH (2 mL) was added methylamine (217.8 mg, 2.1 mmol, 30% in EtOH) at 20° C. After stirring at 20° C. for 2 hr, Pd/C (10 mg, 100.0 mmol, 10% purity) was added under N₂. The suspension was degassed under vacuum and purged with H₂ several times. The reaction mixture was stirred at 20° C. for 12 hrs under H₂ (15 psi). The mixture was filtered and the filtrate was concentrated in vacuum to give the Intermediate compound 39 (100 mg, crude) as yellow oil. ¹H NMR (ET34324-14-P1A, CDCl₃-d, 400 MHz) δ=7.39-7.33 (m, 1H), 7.27-7.19 (m, 2H), 7.12 (d, J=8.0 Hz, 1H), 3.79 (s, 2H), 2.47 (s, 3H).

Intermediate Compound 40:

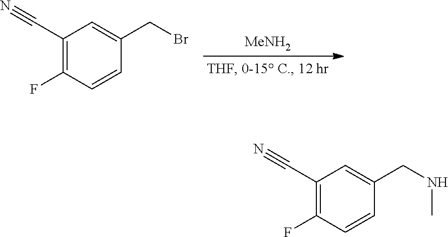

To a solution of MeNH₂ (2 M, 7.01 mL, 14.0 mmol) in THF (6.0 mL) was added 5-(bromomethyl)-2-fluorobenzonitrile (0.5 g, 308.6 μmol) in THF (6.0 mL) at 0° C. under N₂. The reaction mixture was stirred at 15° C. for 12 hrs. The reaction mixture was washed with a saturated solution of NaHCO₃ solution (50 mL) and extracted with ethyl acetate (20 mL, 3 times). The combined organic layers were washed with brine (50 mL), dried over Na₂SO₄, filtered and concentrated under reduced pressure to give the Intermediate compound 40 (0.3 g, yield 78%) as yellow oil. LCMS (ESI position ion) m/z: (M+H)+: 165.1 (calculated: 165.1). ¹H NMR (ET34313-14-P1A, CDCl₃-d, 400 MHz) δ=7.62-7.58 (m, 2H), 7.19-7.15 (m, 1H), 3.76 (s, 2H), 2.44 (s, 3H), 1.89-1.82 (m, 1H)

Intermediate Compound 41:

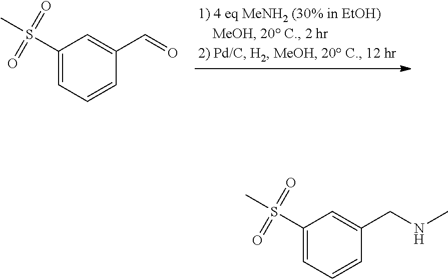

To a solution of 3-(methylsulfonyl)benzaldehyde (400 mg, 2.17 mmol) in MeOH (8 mL) was added methanamine (899.1 mg, 8.6 mmol, 30% in EtOH) and AcOH (13.0 mg, 217.1 μmol). The mixture was stirred at 20° C. for 2 hr. Then Pd/C (50 mg, 2.17 mmol, 10% purity) was added under N₂. The suspension was degassed under vacuum and purged with H₂ several times. The mixture was stirred under H₂ (15 psi) at 20° C. for 12 hours. The mixture was filtered and concentrated in vacuum to give Intermediate compound 41 (400 mg, crude) as yellow oil. ¹H NMR (ET34324-17-P1A, CDCl₃-d, 400 MHz) δ=7.92 (s, 1H), 7.85 (d, J=7.6 Hz, 1H), 7.65 (d, J=7.6 Hz, 1H), 7.54 (d, J=8.0 Hz, 1H), 3.86 (s, 2H), 3.07 (s, 3H), 2.48 (s, 3H)

Intermediate Compound 42:

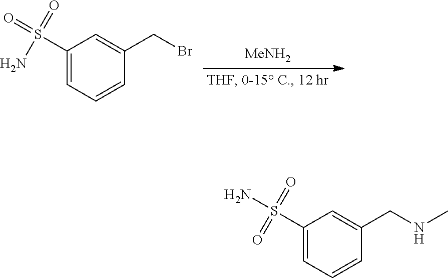

To a solution of MeNH₂ (2 M, 3.0 mL, 6.0 mmol) in THF (3.0 mL) was added 3-(bromomethyl)benzenesulfonamide (230 mg, 919.6 μmol) in THF (3.0 mL) at 0° C. under N₂. The mixture was stirred at 15° C. for 12 hrs. The reaction mixture was washed with Na₂CO₃ solution (20 mL) and extracted with ethyl acetate (20 mL, 3 times). The organic layer was combined and washed with brine (30 mL), dried over Na₂SO₄, filtered and concentrated under reduced pressure to give the Intermediate compound 42 (0.17 g, yield 92%) as colorless oil. LCMS (ESI position ion) m/z: (M+H)+: 201.1 (calculated: 201.1). ¹H NMR (ET34313-29-P1A, DMSO-d₆, 400 MHz) δ=7.87 (s, 2H), 7.79-7.71 (m, 1H), 7.62-7.55 (m, 2H), 3.96 (s, 2H), 2.42 (s, 3H), 2.36 (s, 2H).

Intermediate Compound 43:

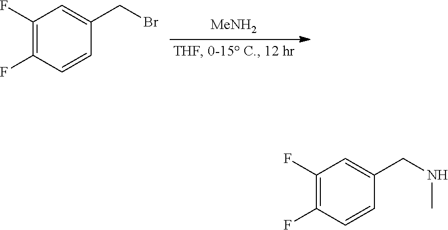

To a solution of MeNH₂ (2 M, 7.25 mL, 14.5 mmol) in THF (6.0 mL) was added 4-(bromomethyl)-1,2-difluorobenzene (0.5 g, 2.42 mmol) at 0° C. under N₂. The mixture was stirred at 15° C. for 12 hrs; The reaction mixture was washed with a saturated solution of NaHCO₃ (50 mL) and extracted with ethyl acetate (20 mL, 3 times). The combined organic layers were washed with brine (50 mL), dried over Na₂SO₄, filtered and concentrated under reduced pressure to give the Intermediate compound 43 (0.35 g, yield 92%) as yellow oil. LCMS (ESI position ion) m/z: (M+H)+: 157.9 (calculated: 158.1). ¹H NMR (ET34313-2-P1A, CDCl₃-d, 400 MHz) δ=7.14-7.07 (m, 2H), 7.04-7.03 (m, 1H), 3.71 (s, 2H), 2.44 (s, 3H).

Intermediate Compound 44:

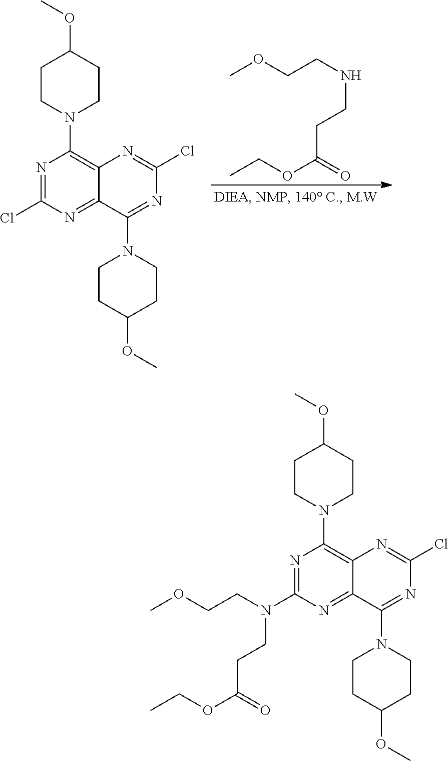 [SMILES: COC1CCN(c2nc(Cl)nc3c(N4CCC(OC)CC4)nc(Cl)nc23)CC1 ; CCOC(=O)CCNCCOC ; CCOC(=O)CCN(CCOC)c1nc(N2CCC(OC)CC2)c2nc(Cl)nc(N3CCC(OC)CC3)c2n1]

A mixture of Intermediate compound 2 (0.7 g, 1.64 mmol, 1 eq), ethyl 3-((2-methoxyethyl)amino)propanoate (718.4 mg, 4.10 mmol, 2.5 eq) and DIEA (847.97 mg, 6.56 mmol, 1.14 mL, 4.0 EQ° in NMP (2 mL) was stirred at 140° C. for 2 h under microwave. The mixture was poured into water (40 mL), extracted with ethyl acetate (100 mL). The organic layer was dried over Na2SO4 and concentrated. The crude was purified by pre-HPLC (column: Waters Xbridge C18 150*50 mm*10 um; mobile phase: [water (10 mM NH4HCO3)-ACN]; B %: 63%-93%, 11.5 min) to give the Intermediate compound 44 (0.56 g, 60% yield) as a light yellow oil. LCMS (ESI position ion) m/z: (M+H)+: 566.5 (calculated: 566.3)

Intermediate Compound 45:

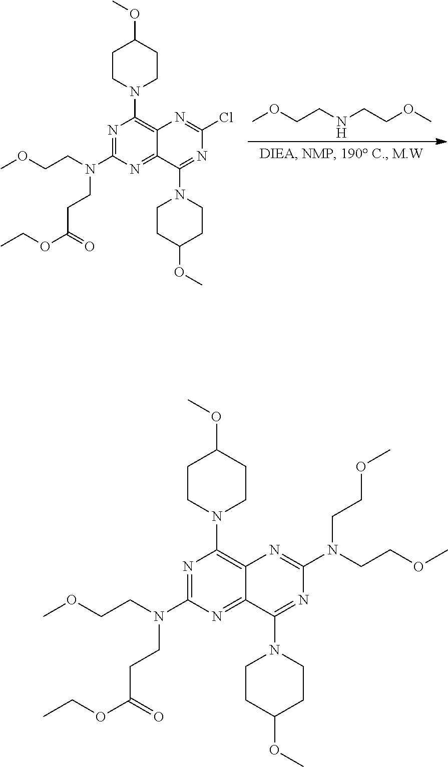

A mixture of the Intermediate compound 44 (560 mg, 989 μmol, 1 eq), bis(2-methoxyethyl)amine (1.19 g, 8.90 mmol, 9 eq) and DIEA (511 mg, 3.95 mmol, 688 μL, 4 eq) in NMP (1.5 mL) was stirred at 190° C. for 2 h under microwave. The mixture was filtered and the filtrate was purified by pre-HPLC (Column: Waters Xbridge C18 150*50 mm*10 um; Condition: water (10 mM NH₄HCO₃)-ACN) to give the Intermediate compound 45 (0.5 g, 69% yield) as a yellow gum. LCMS (ESI position ion) m/z: (M+H)+: 663.5 (calculated: 663.4) Intermediate compound 46:

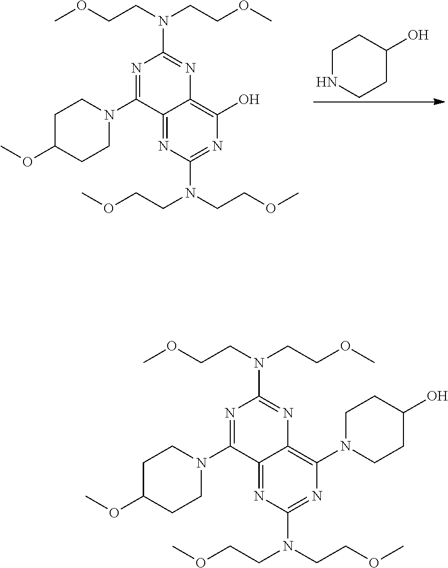

To a solution of Intermediate compound 16 (300 mg, 572.93 μmol, 1 eq), BOP (330 mg, 746.13 μmol, 1.3 eq) and DBU (132 mg, 867.05 μmol, 130.69 μL, 1.51 eq) in DMF (2 mL) was stirred at 0° C. for 0.5 hr. The mixture was added piperidin-4-ol (174 mg, 1.72 mmol, 3 eq) in DMF (0.5 mL) was stirred at 16° C. for 14.5 hrs. The residue was purified by prep-HPLC (column: Waters Xbridge C18 150*50 mm*10 um; mobile phase: [water (10 mM NH4HCO3)-ACN]; B %: 43%-73%, 11.5 min) to give the Intermediate compound 46 (300 mg, crude) as a yellow solid.

Intermediate Compound 47:

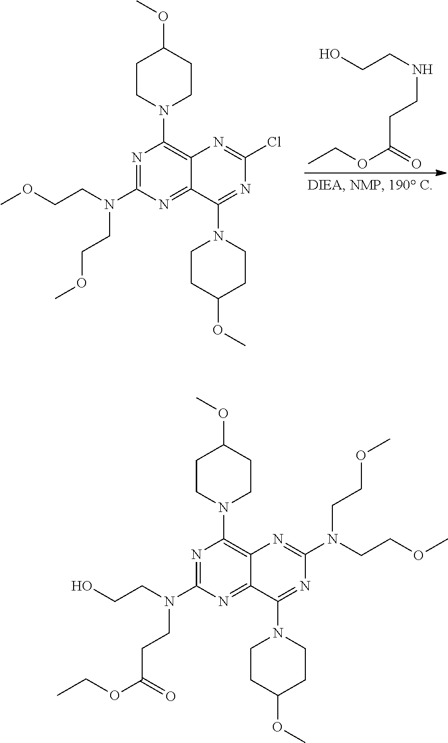

A mixture of intermediate compound 9, ethyl 3-((2-hydroxyethyl)amino)propanoate (615 mg, 3.82 mmol, 2 eq) and DIEA (986 mg, 7.63 mmol, 1.33 mL, 4 eq) in NMP (2 mL) was stirred at 190° C. for 2 h under microwave. The mixture was filtered. The crude was purified by pre-HPLC (Column: Waters Xbridge C18 150*50 mm*10 um; Condition: water (10 mM NH4HCO3)-ACN) to give the intermediate compound 47 (0.2 g, 10% yield, 64% purity) as a light yellow oil.

LCMS (ESI position ion) m/z: (M+H)+: 649.4 (calculated: 648.39)

Intermediate Compound 48:

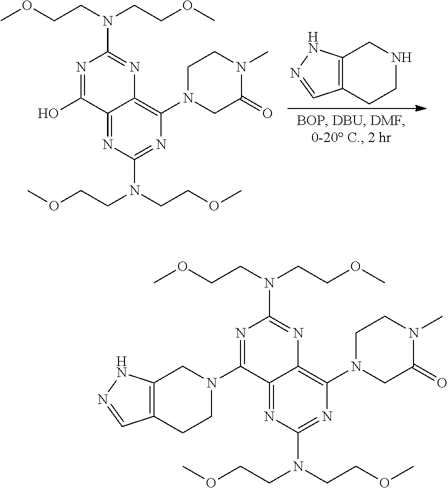

To a solution of intermediate compound 23 (200 mg, 382.70 umol) in dimethyl formamide (1.5 mL) were added BOP (253.89 mg, 574.06 umol) and DBU (524.37 mg, 3.44 mmol) at 0° C. The reaction mixture was stirred at 0° C. for 30 min. After 30 min, 4,5,6,7-tetrahydro-1H-pyrazolo[3,4-c]pyridine (112.56 mg, 574.06 umol, 2 HCl salt) was added and stirred at 20° C. for 1.5 hr. The reaction mixture was evaporated and purified by prep-HPLC (Column: Waters Xbridge BEH C18 100*30 mm*10 um; Condition: water (10 mM NH4HCO3)-ACN) to give the intermediate compound 48 (100 mg, yield 42%) as yellow solid.

1H NMR (400 MHz, CHLOROFORM-d) δ ppm 7.38-7.45 (m, 1H), 4.51-4.63 (m, 2H), 4.24-4.47 (m, 2H), 3.74-3.93 (m, 8H), 3.61 (br d, J=5.26 Hz, 8H), 3.49-3.55 (m, 2H), 3.29-3.43 (m, 14H), 3.07-3.28 (m, 2H), 3.02-3.06 (m, 3H), 2.84-3.00 (m, 2H).

Intermediate Compound 49:

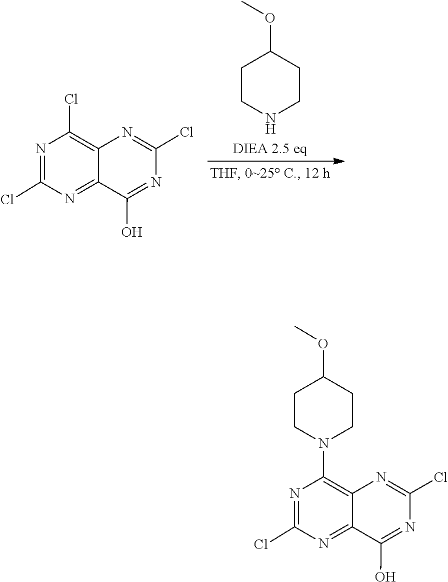

To a solution of intermediate compound 21 (8.5 g, 33.80 mmol, 1 eq) in THF (150 mL) was added DIEA (10.92 g, 84.51 mmol, 14.72 mL, 2.5 eq) and cooled to 0° C. A solution of 4-methoxypiperidine (3.89 g, 33.80 mmol, 1 eq) in THF (20 mL) was dropwise added to the reaction mixture at 0° C. After addition, the mixture was stirred at 25° C. for 12 hr. The reaction mixture was concentrated to dryness. This residue was diluted with aqueous citric acid (20 g/550 mL) and this suspension was stirred at 20° C. for 1 hr. Then the suspension was filtered, and the filter cake was washed by water (50.0 mL*3). The solid was dried by azeotropic dehydration with ACN under vacuum to give the intermediate compound 49 (11.58 g, crude) as yellow solid.

LCMS (ESI position ion) m/z: (M+H)+: 330.0 (calculated: 329.04)

1H NMR (400 MHz, DMSO-d6) δ ppm 4.43 (br s, 2H), 3.83 (br s, 1H), 3.49 (dt, J=3.8, 7.6 Hz, 1H), 3.28 (s, 4H), 1.95 (ddd, J=3.4, 6.7, 9.6 Hz, 2H), 1.63-1.48 (m, 2H).

Intermediate Compounds 50 and 51:

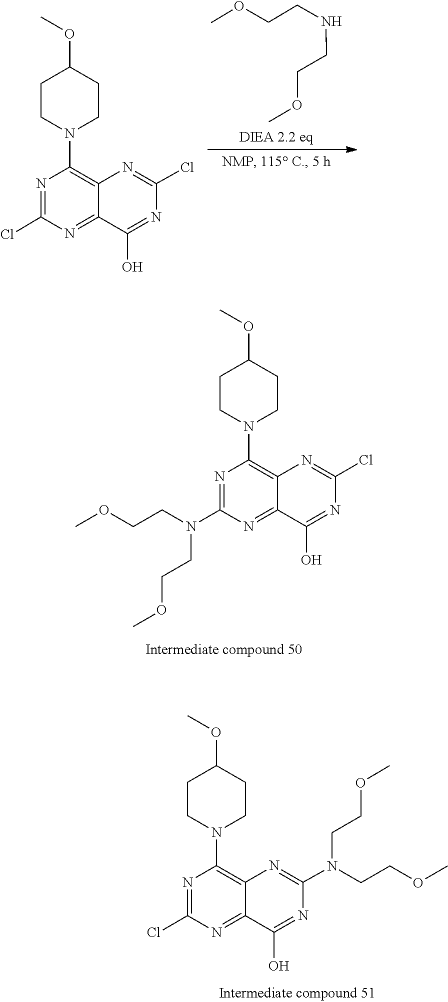

To a solution of intermediate compound 49 (35.8 g, 108.43 mmol, 1 eq) in NMP (110 mL) were added DIEA (30.83 g, 238.54 mmol, 41.55 mL, 2.2 eq) and bis(2-methoxyethyl)amine (14.44 g, 108.43 mmol, 16.01 mL, 1 eq). The mixture was stirred at 115° C. for 5 hr. The reaction mixture was filtered. The filtrate was purified by prep-HPLC (column: Phenomenex luna C18 150*25 mm*10 um; mobile phase: [water (0.225% FA)-ACN];B %: 13%-43%, 10 min). to give the intermediate 50 (7.52 g, 16% yield) as yellow solid and the intermediate compound 51 (17.54 g, 38% yield) as yellow solid.

Intermediate 50:

LCMS (ESI position ion) m/z: (M+H)+: 427.1 (calculated: 426.18)

1H NMR (400 MHz, CHLOROFORM-d) δ ppm 4.88-4.13 (m, 6H), 3.81 (t, J=4.8 Hz, 3H), 3.65 (br s, 3H), 3.59-3.50 (m, 1H), 3.38 (s, 9H), 2.03-1.93 (m, 2H), 1.76 (ttd, J=3.5, 7.1, 10.3 Hz, 2H).

Intermediate 51:

LCMS (ESI position ion) m/z: (M+H)+: 427.1 (calculated: 426.18)

1H NMR (400 MHz, CHLOROFORM-d) δ ppm 10.52 (br s, 1H), 4.55 (br s, 2H), 3.83 (br s, 2H), 3.71-3.66 (m, 4H), 3.65-3.60 (m, 4H), 3.51 (tt, J=3.7, 7.6 Hz, 1H), 3.40 (s, 9H), 2.04-1.94 (m, 2H), 1.76-1.63 (m, 2H).

Intermediate Compound 52:

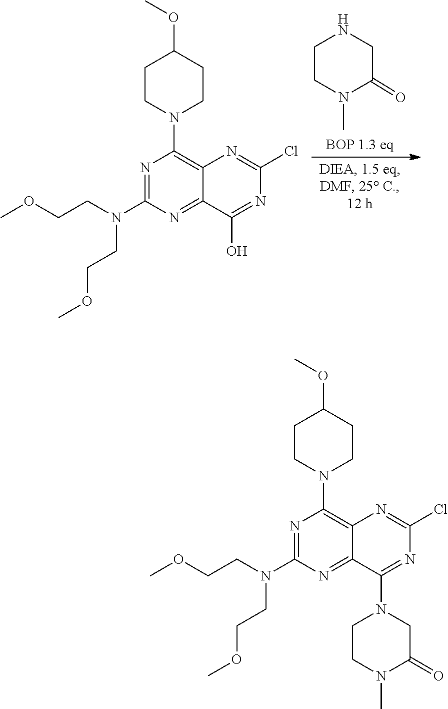

To a solution of intermediate compound 50 (2.3 g, 5.39 mmol, 1 eq) in DMF (23 mL) were added DIEA (1.04 g, 8.08 mmol, 1.41 mL, 1.5 eq) and BOP (3.10 g, 7.00 mmol, 1.3 eq) at 0° C. The mixture was stirred at 0° C. for 0.5 hr. After 0.5 hr, 1-methylpiperazin-2-one (1.84 g, 16.16 mmol, 3 eq) was added to the mixture. The mixture was stirred at 20° C. for 12 hr. To the reaction mixture was added water (100 mL) and extracted with Ethyl acetate (80 mL×2). The organic layer was washed with brine, dried by Na2SO4. The solution was concentrated to give a residue. The residue was purified by basic preparative HPLC to give the intermediate compound 52 (2.1 g, 74% yield).

LCMS (ESI position ion) m/z: (M+H)+: 523.2 (calculated: 522.24)

1H NMR (400 MHz, CHLOROFORM-d) δ ppm 4.88 (br s, 2H), 4.68-4.42 (m, 4H), 3.83-3.69 (m, 6H), 3.58 (br t, J=5.9 Hz, 4H), 3.53-3.48 (m, 3H), 3.38 (s, 3H), 3.35 (s, 6H), 3.01 (s, 3H), 1.98 (ddd, J=3.1, 6.5, 9.6 Hz, 2H), 1.70-1.59 (m, 2H).

Intermediate Compound 53:

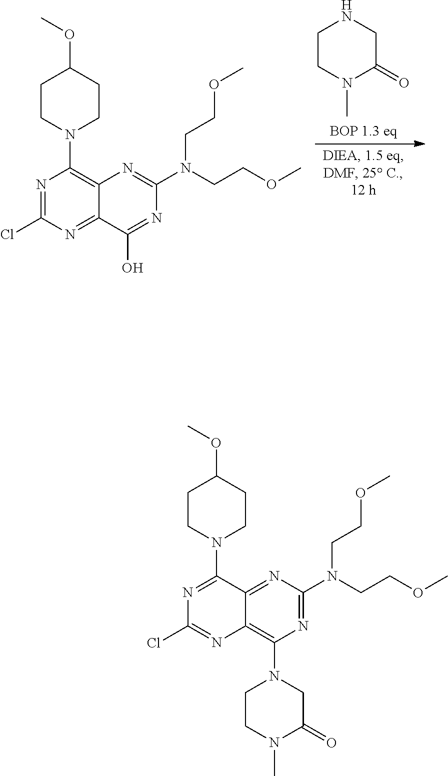

To a solution of intermediate compound 51 (5 g, 11.71 mmol, 1 eq) in DMF (50 mL) were added DIEA (2.27 g, 17.57 mmol, 3.06 mL, 1.5 eq) and BOP (6.73 g, 15.23 mmol, 1.3 eq) at 0° C. The mixture was stirred at 0° C. for 0.5 hr. After 0.5 hr, 1-methylpiperazin-2-one (4.01 g, 35.14 mmol, 3 eq) was added to the mixture. The mixture was stirred at 20° C. for 12 hr. The reaction mixture was added water (100 mL) and extracted with Ethyl acetate (100 mL×3). The organic layer was washed with brine, dried by Na2SO4. The solution was concentrated to give a residue. The residue was purified by column chromatography (SiO2, Petroleum ether/Ethyl acetate=1/1 to 0/1) to give the intermediate compound 53 (6.3 g, crude).

LCMS (ESI position ion) m/z: (M+H)+: 523.1 (calculated: 522.24)

1H NMR (400 MHz, CHLOROFORM-d) δ ppm 4.71-4.46 (m, 5H), 4.01-3.68 (m, 6H), 3.60-3.47 (m, 8H), 3.39 (s, 3H), 3.34 (s, 6H), 3.07-2.97 (m, 3H), 2.05-1.96 (m, 2H), 1.75-1.65 (m, 2H)

Intermediate Compound 54:

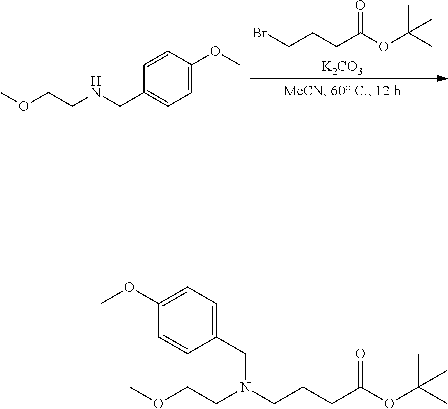

A mixture of 2-methoxy-N-(4-methoxybenzyl)ethan-1-amine (8 g, 40.97 mmol, 1 eq), tert-butyl 4-bromobutanoate (9.14 g, 40.97 mmol, 1 eq) and K2CO3 (16.99 g, 122.91 mmol, 3 eq) in MeCN (100 mL) was stirred at 60° C. for 12 hr. The reaction mixture was filtered, the filtrate was concentrated under reduced pressure to give a residue. The residue was purified by column on silica (petroleum ether:ethyl acetate=10:1˜3:1) and concentrated under reduced pressure to give the intermediate compound 54 (10.3 g, 68% yield) as colorless oil.

LCMS (ESI position ion) m/z: (M+H)+: 338.3 (calculated: 337.22)

1H NMR (400 MHz, CHLOROFORM-d) δ ppm 7.23 (d, J=8.6 Hz, 2H), 6.84 (d, J=8.6 Hz, 2H), 3.79 (s, 3H), 3.56 (s, 2H), 3.44 (t, J=6.2 Hz, 2H), 3.31 (s, 3H), 2.64 (t, J=6.2 Hz, 2H), 2.48 (t, J=7.2 Hz, 2H), 2.22 (t, J=7.5 Hz, 2H), 1.79-1.71 (m, 2H), 1.42 (s, 9H)

Intermediate Compound 55:

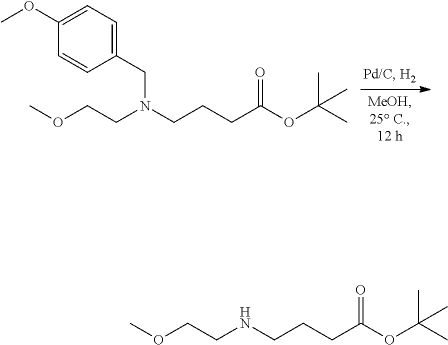

To a solution of intermediate compound 54 (8 g, 23.71 mmol, 1 eq) in MeOH (150 mL) was added wet Pd/C (8.00 g, 3.76 mmol, 5% purity) under N2. The suspension was degassed under vacuum and purged with H2 several times. The mixture was stirred under H2 (45 psi) at 25° C. for 12 hr. The reaction mixture was filtered. The filter cake was washed with MeOH (80 mL×4). The filtrate was concentrated to give the intermediate compound 55 (5.6 g, crude) as colorless liquid.

1H NMR (400 MHz, CHLOROFORM-d) δ ppm 3.52-3.47 (m, 2H), 3.36 (s, 3H), 2.82-2.75 (m, 2H), 2.65 (t, J=7.2 Hz, 2H), 2.29-2.25 (m, 2H), 1.78 (quin, J=7.3 Hz, 2H), 1.44 (s, 9H)

Intermediate Compound 56 and 57:

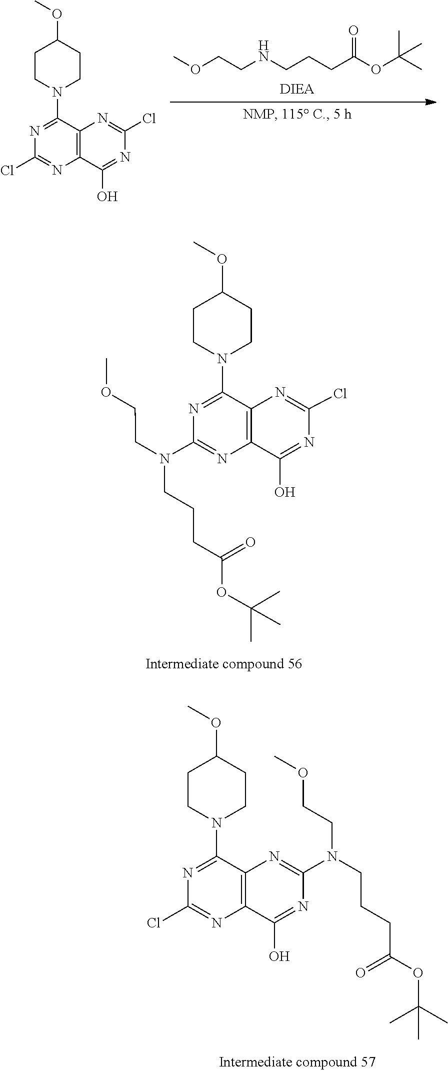 [SMILES: COC1CCN(c2nc(Cl)nc3c(O)nc(Cl)nc23)CC1 ; COCCNCCCC(=O)OC(C)(C)C ; COCCN(CCCC(=O)OC(C)(C)C)c1nc(N2CCC(OC)CC2)c2nc(Cl)nc(O)c2n1 ; COCCN(CCCC(=O)OC(C)(C)C)c1nc(O)c2nc(Cl)nc(N3CCC(OC)CC3)c2n1]

To a solution of intermediate compound 49 (1 g, 3.03 mmol, 1 eq) in NMP (5 mL) was added DIEA (782.89 mg, 6.06 mmol, 1.06 mL, 2 eq) and the intermediate compound 55 (855.61 mg, 3.94 mmol, 1.3 eq). The mixture was stirred at 115° C. for 5 hr. The reaction mixture was filtered. The filtrate was purified by prep-HPLC (column: Phenomenex luna C18 150*40 mm*15 um; mobile phase: [water (0.225% FA)-ACN];B %: 45%-75%, 10 min) to give the intermediate compound 56 (180 mg, 12% yield) as white solid and the intermediate compound 57 (560 mg, 36% yield) as white solid.

Intermediate 56:

LCMS (ESI position ion) Rt=0.868 min, m/z: (M+H)+: 511.3 (calculated: 510.23)

Intermediate 57:

LCMS (ESI position ion) Rt=0.952 min, m/z: (M+H)+: 511.3 (calculated: 510.23)

Intermediate Compound 58:

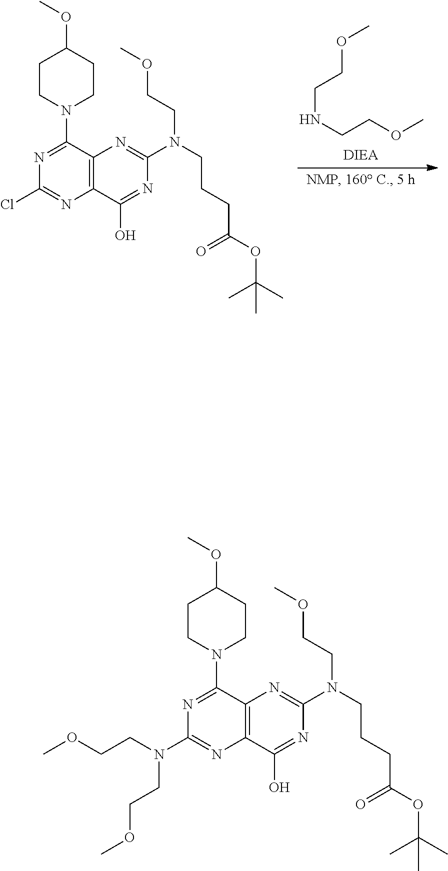

Intermediate compound 57 (210 mg, 410.95 umol, 1 eq), 2-methoxy-N-(2-methoxyethyl)ethanamine (547.34 mg, 4.11 mmol, 606.80 uL, 10 eq) and DIEA (637.35 mg, 4.93 mmol, 858.96 uL, 12 eq) were taken up into a microwave tube in NMP (2 mL). The sealed tube was heated at 160° C. for 5 hr under microwave. The reaction mixture was filtered. The filtrate was purified by prep-HPLC (column: Phenomenex Gemini-NX C18 75*30 mm*3 um; mobile phase: [water (0.225% FA)-ACN];B %: 25%-55%, 7 min) to give the intermediate compound 58 (65 mg, 26% yield) as a yellow solid.

LCMS (ESI position ion) Rt=0.876 min, m/z: (M+H)+: 608.4 (calculated: 607.37)

Intermediate Compound 59:

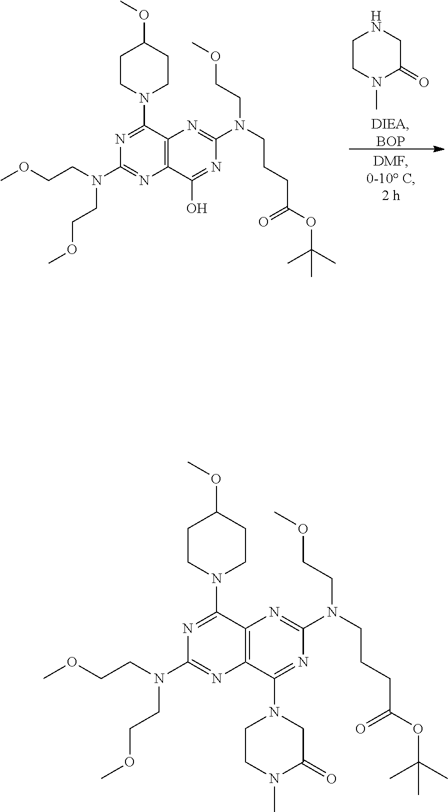

To a solution of intermediate compound 58 (60 mg, 98.73 umol, 1 eq) in DMF (2 mL) were added DIEA (19.14 mg, 148.09 umol, 25.79 uL, 1.5 eq) and BOP (56.76 mg, 128.34 umol, 1.3 eq) at 0° C. The mixture was stirred at 0° C. for 0.5 hr. After 0.5 hr, 1-methylpiperazin-2-one (33.81 mg, 296.18 umol, 3 eq) was added to the mixture. The mixture was stirred at 10° C. for 2 hr. LCMS showed starting material was consumed completely and desired mass was detected. The reaction mixture was added water (30 mL) and extracted with Ethyl acetate (20 mL×3). The organic layer was washed with brine, dried by Na2SO4. The solution was concentrated to give the intermediate compound 59 (70 mg, crude) as yellow gum.

LCMS (ESI position ion) Rt=0.887 min, m/z: (M+H)+: 704.4 (calculated: 703.44)

Intermediate Compound 60:

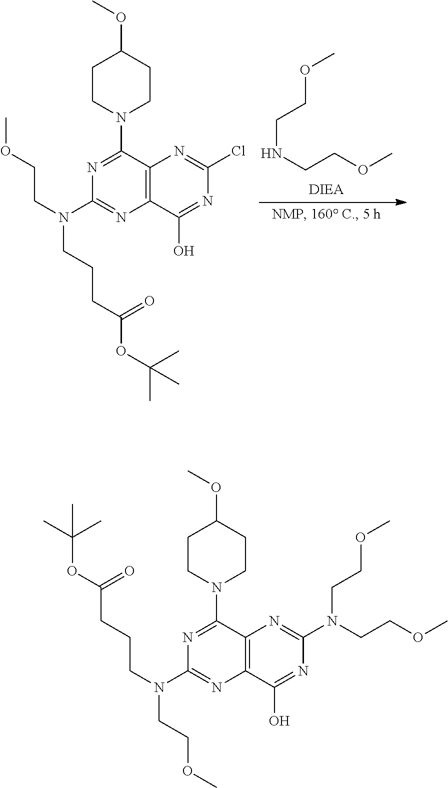

Intermediate compound 56 (180 mg, 352.24 umol, 1 eq), 2-methoxy-N-(2-methoxyethyl)ethanamine (469.14 mg, 3.52 mmol, 520.12 uL, 10 eq) and DIEA (546.30 mg, 4.23 mmol, 736.25 uL, 12 eq) were taken up into a microwave tube in NMP (2 mL). The sealed tube was heated at 160° C. for 5 hr under microwave. The reaction mixture was filtered. The filtrate was purified by prep-HPLC (column: Phenomenex Gemini-NX C18 75*30 mm*3 um; mobile phase: [water (0.225% FA)-ACN];B %: 25%-55%, 7 min) to give the intermediate compound 60 (160 mg, 74% yield) as yellow solid.

LCMS (ESI position ion) Rt=0.868 min, m/z: (M+H)+: 608.6 (calculated: 607.37)

Intermediate Compound 61:

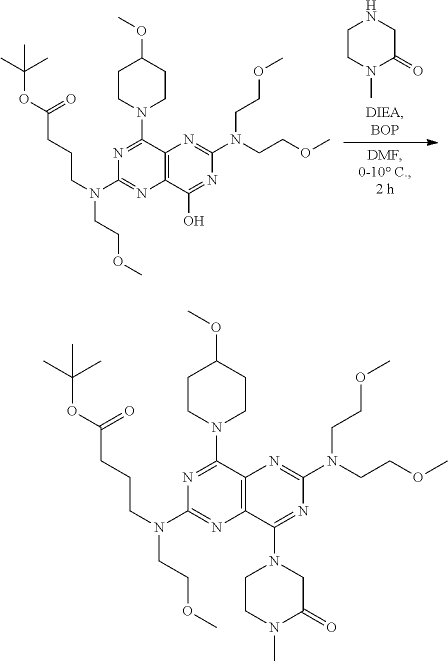

To a solution of intermediate compound 60 (80 mg, 131.64 umol, 1 eq) in DMF (3 mL) were added DIEA (25.52 mg, 197.45 umol, 34.39 uL, 1.5 eq) and BOP (75.69 mg, 171.13 umol, 1.3 eq) at 0° C. The mixture was stirred at 0° C. for 0.5 hr. After 0.5 hr, 1-methylpiperazin-2-one (45.08 mg, 394.91 umol, 3 eq) was added to the mixture. The mixture was stirred at 10° C. for 2 hr. The reaction mixture was added water (50 mL) and extracted with Ethyl acetate (30 mL×3). The organic layer was washed with brine, dried by Na2SO4. The solution was concentrated to give the intermediate compound 61 (90 mg, 97% yield) as yellow gum.

LCMS (ESI position ion) Rt=0.901 min, m/z: (M+H)+: 704.5 (calculated: 703.44)

Intermediate Compound 62:

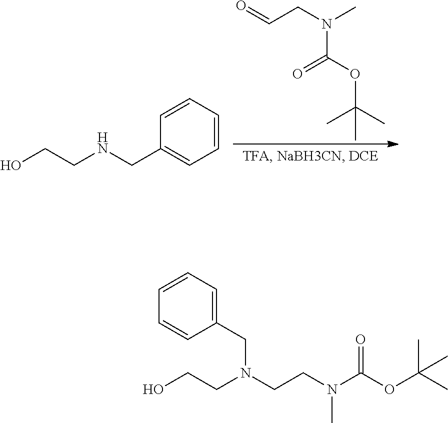 [SMILES: OCCNCc1ccccc1 ; CN(CC=O)C(=O)OC(C)(C)C ; CN(CCN(CCO)Cc1ccccc1)C(=O)OC(C)(C)C]

A mixture of 2-(benzylamino)ethanol (300 mg, 1.98 mmol, 280.37 uL, 1 eq), tert-butyl N-methyl-N-(2-oxoethyl)carbamate (378.02 mg, 2.18 mmol, 1.1 eq) NaBH3CN (187.02 mg, 2.98 mmol, 1.5 eq), TFA (678.68 mg, 5.95 mmol, 440.70 uL, 3 eq) in DCE (3 mL) was stirred at 25° C. for 12 h. The reaction mixture was diluted with water (50 ml), extracted with Ethyl acetate (25×2 mL), The combined organic layers were washed with 50 mL brine, dried over Na2SO4 and filtered, The filtrate was concentrated under reduced pressure to give the intermediate compound 62 (790 mg, crude) as a colourless oil.

LCMS (ESI position ion) m/z: (M+H)+: 309.3 (calculated: 308.21)

1H NMR (400 MHz, CHLOROFORM-d) δ ppm 7.54-7.45 (m, 5H), 4.54-4.35 (m, 2H), 4.01-3.85 (m, 2H), 3.83-3.62 (m, 2H), 3.57 (br d, J=15.2 Hz, 2H), 3.32 (br s, 2H), 2.88 (s, 3H), 1.49-1.46 (m, 9H)

Intermediate Compound 63:

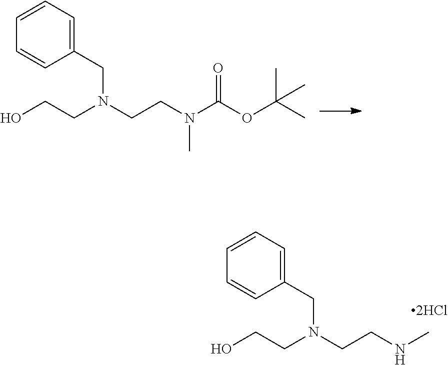 [SMILES: CN(CCN(CCO)Cc1ccccc1)C(=O)OC(C)(C)C ; CNCCN(CCO)Cc1ccccc1]

A mixture of intermediate compound 62 (790 mg, 2.56 mmol, 1 eq) in HCl/dioxane (4 M, 10.25 mL, 16 eq) was stirred at 25° C. for 12 h. The mixture was concentrated under reduced pressure to give the intermediate compound 63 (750 mg, crude) as a white oil.

LCMS (ESI position ion) m/z: (M+H)+: 209.2 (calculated: 208.16)

Intermediate Compound 64:

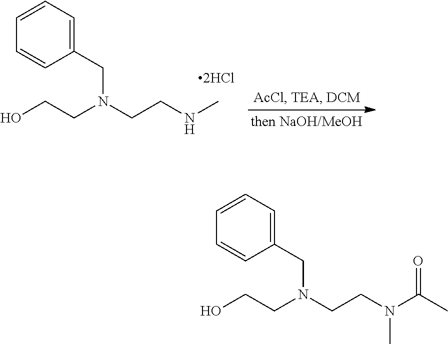 [SMILES: CNCCN(CCO)Cc1ccccc1 ; CC(=O)N(C)CCN(CCO)Cc1ccccc1]

To a solution of intermediate compound 63 (650 mg, 3.12 mmol, 1 eq) in DCM (9 mL) was added TEA (1.58 g, 15.60 mmol, 2.17 mL, 5 eq) and acetyl chloride (367.43 mg, 4.68 mmol, 334.03 uL, 1.5 eq) with stirring at 5° C. for 2 h. The reaction mixture was diluted with 100 ml water, extracted with dichloromethane (50 mL×2). The combined organic layers were washed with 50 mL brine, dried over Na2SO4 and filtered. The filtrate was concentrated under reduced pressure. The residue was dissolved in methanol (20 mL) and NaOH in water (41.04 mg, 1.03 mmol, 5 mL) was added. The reaction mixture was stirred at 25° C. for 12 h. The reaction mixture was diluted with 50 ml water, extracted with dichloromethane (25 mL×2). The combined organic layers were washed with 25 mL brine, dried over Na₂SO₄ and filtered. The filtrate was concentrated under reduced pressure to give a colourless. The crude product was purified by prep-HPLC (column: Phenomenex Luna C18 150*25 mm*10 um; mobile phase: [water (0.225% FA)-ACN];B %: 14%-44%, 10 min) to give the intermediate compound 64 (220 mg, 85% yield) as a colourless oil.

1H NMR (400 MHz, CHLOROFORM-d) δ ppm 7.36-7.29 (m, 5H), 3.68-3.65 (m, 2H), 3.63-3.55 (m, 2H), 3.49-3.39 (m, 2H), 2.78-2.74 (m, 2H), 2.73-2.59 (m, 2H), 2.59-2.54 (m, 3H), 2.03 (s, 3H)

Intermediate Compound 65:

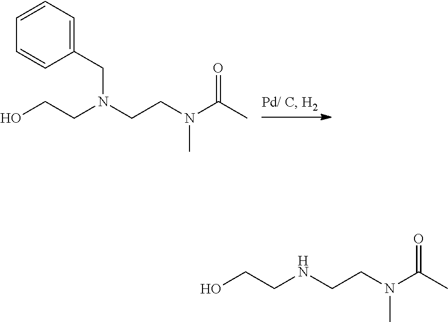 [SMILES: CC(=O)N(C)CCN(CCO)Cc1ccccc1 ; CC(=O)N(C)CCNCCO]

A mixture of intermediate compound 64 (220 mg, 878.82 umol, 1 eq) and Pd/C (100 mg, 10% purity) in EtOH (5 mL) was degassed and purged with H2 for three times. The resulting mixture was stirred at 25° C. for 16 h under H2 (15 psi). The mixture was filtered and the filtrate was concentrated. The residue was purified by preparative TLC (Dichloromethane:Methanol=10:1) to give the intermediate compound 65 (120 mg, crude) as a colorless gum.

Intermediate Compound 66:

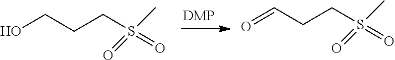

To a solution of 3-methylsulfonylpropan-1-ol compound 1 (5 g, 36.18 mmol, 1 eq) in DCM (50 mL) was added the Dess Martin periodinane (18.42 g, 43.42 mmol, 13.44 mL, 1.2 eq). The reaction was stirred at 25° C. for 2 hr. The reaction mixture was filtered and concentrate under vacuum. The residue was purified by silica gel column chromatography (ethyl acetate 100%) to give the 3-methylsulfonylpropanal (1.4 g, 28.41% yield) as a yellow liquid.

1H NMR (400 MHz, CHLOROFORM-d) δ ppm 9.93-9.72 (m, 1H), 3.45-3.29 (m, 2H), 3.22-3.05 (m, 2H), 3.04-2.93 (m, 3H)

Intermediate Compound 67:

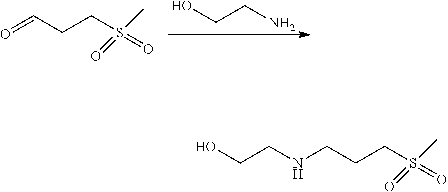

To a mixture of 2-aminoethanol (134.57 mg, 2.20 mmol, 133.24 uL, 1 eq), intermediate compound 66 (300 mg, 2.20 mmol, 266.75 uL, 1 eq), and DCE (5 mL), was added acetic acid (396.90 mg, 6.61 mmol, 378.00 uL, 3 eq), NaBH3CN (207.67 mg, 3.30 mmol, 1.5 eq) in portions. The reaction mixture was stirred at 25° C. for 2 hrs. The reaction mixture was concentrated under vacuum. The residue was purified by pre-HPLC (column: Waters Atlantis T3 150*30 mm*5 um; mobile phase: [water (0.225% FA)-ACN];B %: 1%-20%, 10 min) to give the intermediate compound 67 (150 mg, 37% yield) as an off white solid.

LCMS (ESI position ion) m/z: (M+H)+: 182.1 (calculated: 181.08)

1H NMR (400 MHz, METHANOL-d4) δ ppm 3.77-3.67 (m, 2H), 3.25-3.05 (m, 6H), 2.96 (s, 3H), 2.16 (t, J=7.6 Hz, 2H)

Intermediate Compound 68:

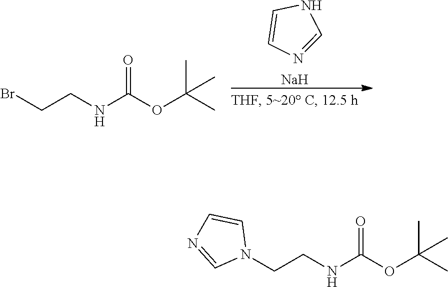

To a solution of imidazole (2 g, 29.38 mmol, 1 eq) in THF (50 mL) was added NaH (1.41 g, 35.25 mmol, 60% purity, 1.2 eq) at 5° C. After addition, the mixture was stirred at this temperature for 0.5 hr, and then tert-butyl (2-bromoethyl)carbamate (6.58 g, 29.38 mmol, 1 eq) was added to the mixture. The resulting mixture was stirred at 20° C. for 12 h. The reaction mixture was partitioned between H2O (300 mL) and Ethyl acetate (300 mL). The organic phase was separated, washed with H2O (200 mL×3), dried over Na2SO4, filtered and concentrated under reduced pressure to give a residue. The residue was purified by basic reversed phase chromatography (NH3.H2O condition) to give the intermediate compound 68 (2.3 g, 37% yield) as a white solid.

LCMS (ESI position ion) m/z: (M+H)+: 212.2 (calculated: 211.13)

Intermediate Compound 69:

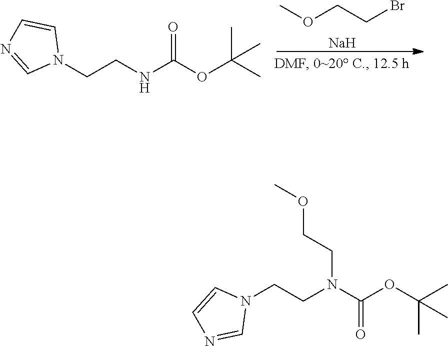

To a solution of intermediate compound 68 (1.4 g, 6.63 mmol, 1 eq) in DMF (20 mL) was added NaH (530.10 mg, 13.25 mmol, 60% purity, 2 eq). The mixture was stirred at 0° C. for 0.5 h. Then 1-bromo-2-methoxyethane (2.76 g, 19.88 mmol, 1.87 mL, 3 eq) was added to the mixture, the mixture was stirred at 20° C. for 12 h. The reaction mixture was partitioned between water 100 mL and Ethyl acetate 150 mL. The organic phase was separated, washed with H2O (100 mL×3), dried over Na2SO4, filtered and concentrated under reduced pressure. The residue was purified by basic reversed phase chromatography to give the intermediate compound 69 (1.5 g, crude) as a colorless oil.

LCMS (ESI position ion) m/z: (M+H)+: 270.2 (calculated: 269.17)

Intermediate Compound 70:

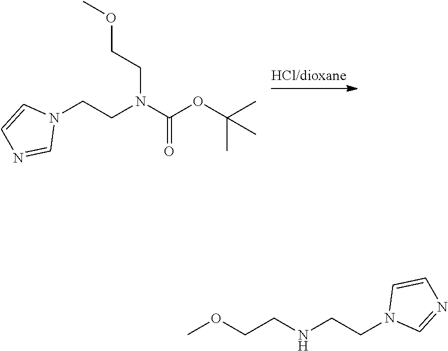 [SMILES: COCCN(CCn1ccnc1)C(=O)OC(C)(C)C ; COCCNCCn1ccnc1]

To a solution of intermediate compound 69 (1.3 g, 4.83 mmol, 1 eq) in HCl/dioxane (4 M, 12.07 mL, 10 eq), the mixture was stirred at 20° C. for 3 h. The reaction mixture was concentrated under reduced pressure to remove solvent to give the intermediate compound 70 (1.0 g, crude) as a yellow oil.

LCMS (ESI position ion) m/z: (M+H)+: 170.2 (calculated: 169.12)

Intermediate Compound 71:

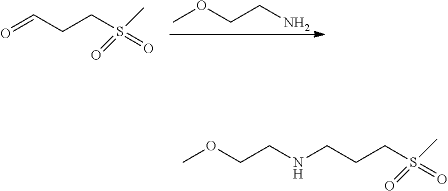

To the mixture of 2-methoxyethanamine (165.48 mg, 2.20 mmol, 1 eq), and intermediate compound 66 (300 mg, 2.20 mmol, 1 eq) in DCE (5 mL) was added acetic acid (396.90 mg, 6.61 mmol, 378.00 uL, 3 eq) and NaBH3CN (207.67 mg, 3.30 mmol, 1.5 eq) in portions. The reaction mixture was stirred at 25° C. for 2 hrs. The reaction mixture was concentrated under vacuum. The residue was purified by pre-HPLC (column: Waters Atlantis T3 150*30 mm*5 um; mobile phase: [water (0.225% FA)-ACN];B %: 1%-20%, 10 min) to give the intermediate compound 71 (150 mg, 35% yield) as an off white solid.

LCMS (ESI position ion) m/z: (M+H)+: 196.2 (calculated: 195.09)

1H NMR (400 MHz, CHLOROFORM-d) δ ppm 3.57-3.51 (m, 2H), 3.33 (s, 3H), 3.20-3.13 (m, 2H), 3.10-2.97 (m, 4H), 2.92 (s, 3H), 2.23-2.14 (m, 2H)

Intermediate Compound 72:

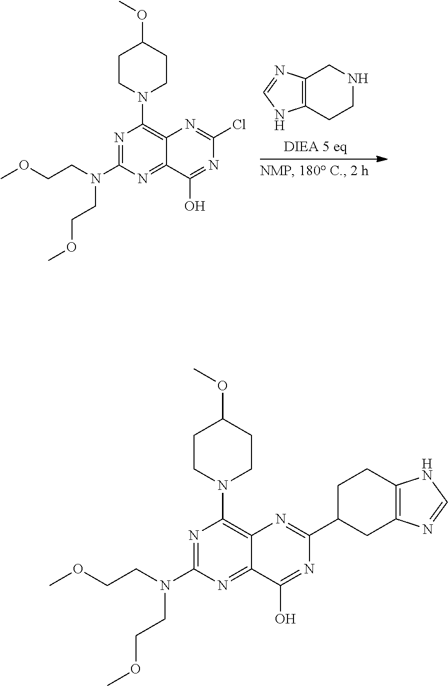

Intermediate compound 50 (200 mg, 468.50 umol, 1 eq), 4,5,6,7-tetrahydro-1H-imidazo[4,5-c]pyridine (230.79 mg, 1.87 mmol, 4 eq) and DIEA (302.75 mg, 2.34 mmol, 408.02 uL, 5 eq) were taken up into a microwave tube in NMP (2 mL). The sealed tube was heated at 180° C. for 2 hr under microwave. The reaction mixture was filtered. The filtrate was purified by prep-HPLC (column: Phenomenex luna C18 150*25 mm*10 um; mobile phase: [water (0.225% FA)-ACN];B %: 13%-43%, 10 min) to give the intermediate compound 72 afford (80 mg, 33% yield) as yellow gum.

LCMS (ESI position ion) m/z: (M+H)+: 514.2 (calculated: 513.28).

Intermediate Compound 73:

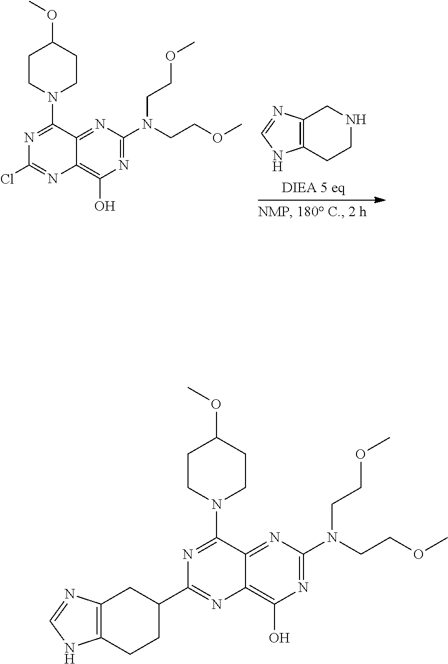

Intermediate compound 51 (200 mg, 468.50 umol, 1 eq), 4,5,6,7-tetrahydro-1H-imidazo[4,5-c]pyridine (230.79 mg, 1.87 mmol, 4 eq) and DIEA (302.75 mg, 2.34 mmol, 408.02 uL, 5 eq) were taken up into a microwave tube in NMP (2 mL). The sealed tube was heated at 180° C. for 2 hr under microwave. The reaction mixture was filtered. The filtrate was purified by prep-HPLC (column: Phenomenex luna C18 150*25 mm*10 um; mobile phase: [water(0.225% FA)-ACN];B %: 13%-43%, 10 min) to give the intermediate compound 73 (140 mg, 58% yield) as a yellow solid.

LCMS (ESI position ion) m/z: (M+H)+: 514.3 (calculated: 513.28)

Intermediate Compound 74:

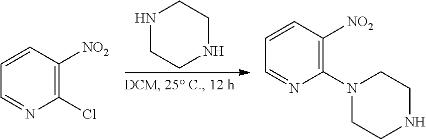 [SMILES: O=[N+]([O-])c1cccnc1Cl ; C1CNCCN1 ; O=[N+]([O-])c1cccnc1N1CCNCC1]

To a solution of piperazine (1.50 g, 17.41 mmol, 2.76 eq) in DCM (30 mL) was slowly added 2-chloro-3-nitropyridine (1 g, 6.31 mmol, 1 eq). The mixture was stirred at 25° C. for 12 hr. The reaction mixture was washed with water (60 mL×3). The organic layer was washed with brine, dried by Na₂SO₄ and concentrated. The residue was purified by basic prep-HPLC to give the intermediate compound 74 (1 g, 76% yield) as a yellow solid.

LCMS (ESI position ion) m/z: (M+H)+: 209.1 (calculated: 208.10)

1H NMR (400 MHz, CHLOROFORM-d) δ ppm 8.32 (dd, J=1.6, 4.5 Hz, 1H), 8.11 (dd, J=1.5, 8.0 Hz, 1H), 6.76-6.69 (m, 1H), 3.45-3.39 (m, 4H), 3.00-2.94 (m, 4H).

Intermediate Compound 75:

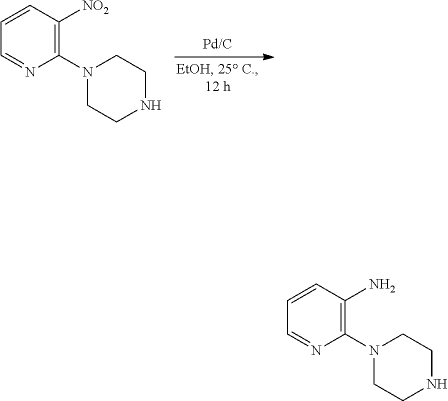 [SMILES: O=[N+]([O-])c1cccnc1N1CCNCC1 ; Nc1cccnc1N1CCNCC1]

To a solution of intermediate compound 74 (500 mg, 2.40 mmol, 1 eq) in EtOH (20 mL) was added wet Pd/C (511.10 mg, 240.13 umol, 5% purity, 0.1 eq) under N2. The suspension was degassed under vacuum and purged with H2 several times. The mixture was stirred under H2 (40 psi) at 25° C. for 12 hr. The reaction mixture was filtered. The filter cake was washed with MeOH (50 mL×3). The filtrate was concentrated and the residue was purified by prep-HPLC (column: Phenomenex luna C18 150*25 mm*10 um; mobile phase: [water(0.225% FA)-ACN];B %: 13%-43%, 10 min) to give the intermediate compound 75 (390 mg, 72% yield) as a white solid.

LCMS (ESI position ion) m/z: (M+H)+: 179.1 (calculated: 178.12)

1H NMR (400 MHz, CHLOROFORM-d) δ ppm 8.51 (s, 1H), 7.84-7.76 (m, 1H), 7.03-6.95 (m, 1H), 6.93-6.84 (m, 1H), 3.49-3.25 (m, 8H).

Intermediate Compound 76:

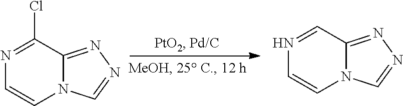 [SMILES: Clc1nccn2cnnc12 ; c1cn2cnnc2cn1]

To a solution of 8-chloro-[1,2,4]triazolo[4,3-a]pyrazine (1 g, 6.47 mmol, 1 eq) in MeOH (20 mL) were added wet Pd/C (413.13 mg, 194.10 umol, 5% purity, 0.03 eq) and PtO2 (0.5 g, 2.20 mmol, 0.34 eq) under N2. The suspension was degassed under vacuum and purged with H2 several times. The mixture was stirred under H2 (50 psi) at 25° C. for 12 hr. The reaction mixture was filtered. The filter cake was washed with MeOH (50 mL×4). The filtrate was concentrated to give the intermediate compound 76 (990 mg, crude) as a red gum.

1H NMR (400 MHz, METHANOL-d4) δ ppm 8.76 (s, 1H), 4.69 (s, 2H), 4.52-4.46 (m, 2H), 3.81-3.74 (m, 2H).

Intermediate Compound 77:

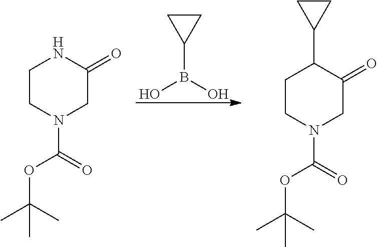 [SMILES: CC(C)(C)OC(=O)N1CCNC(=O)C1 ; OB(O)C1CC1 ; CC(C)(C)OC(=O)N1CCC(C2CC2)C(=O)C1]

To a solution of tert-butyl 3-oxopiperazine-1-carboxylate compound 1 (1 g, 4.99 mmol, 1 eq), DMAP (1.83 g, 14.98 mmol, 3 eq), Cu(OAc)2 (226.78 mg, 1.25 mmol, 0.25 eq), NaHMDS (1 M, 4.99 mL, 1 eq), cyclopropylboronic acid compound 2 (857.97 mg, 9.99 mmol, 2 eq) in toluene (20 mL) was stirred at 95° C. for 1 8 h under oxygen. The reaction mixture was diluted with 50 mL ammonia chloride solution, extracted with Ethyl acetate (50 mL×2). The combined organic layers were washed with 50 mL brine, dried over Na2SO4 and filtered, and concentrated under vacuum. The crude product was purified by prep-HPLC (column: Phenomenex luna C18 150*40 mm*15 um; mobile phase: [water(0.225% FA)-ACN];B %: 19%-49%, 10 min), to give the tert-butyl 4-cyclopropyl-3-oxo-piperazine-1-carboxylate (348 mg, 29% yield) as a yellow oil.

LCMS (ESI position ion) m/z: (M+H)+: 241.2 (calculated: 240.15)

1H NMR (400 MHz, CHLOROFORM-d) δ ppm 4.13-3.92 (m, 2H), 3.70-3.51 (m, 2H), 3.41-3.18 (m, 2H), 2.86-2.65 (m, 1H), 1.47-1.43 (m, 9H), 0.94-0.80 (m, 2H), 0.72-0.57 (m, 2H)

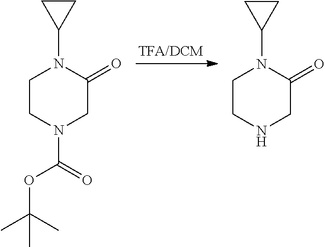 [SMILES: CC(C)(C)OC(=O)N1CCN(C2CC2)C(=O)C1 ; O=C1CNCCN1C1CC1]

To a solution of tert-butyl 4-cyclopropyl-3-oxo-piperazine-1-carboxylate (348 mg, 1.45 mmol, 1 eq) in DCM (3.5 mL) was added TFA (1.08 g, 9.45 mmol, 0.7 mL, 6.53 eq) with stirring at 25° C. for 2 h. The mixture was concentrated under vacuum. The yellow oil (500 mg, crude) was treated by removed TFA resin to pH about 8, filtered, to give to give the intermediate compound 77 (128 mg, 63% yield) as light brown oil.

LCMS (ESI position ion) m/z: (M+H)+: 140.8 (calculated: 140.09)

1H NMR (400 MHz, CHLOROFORM-d) δ ppm 4.02-3.85 (m, 2H), 3.68-3.41 (m, 4H), 2.81-2.64 (m, 1H), 1.32-1.24 (m, 1H), 0.97-0.60 (m, 4H)

Intermediate Compound 78:

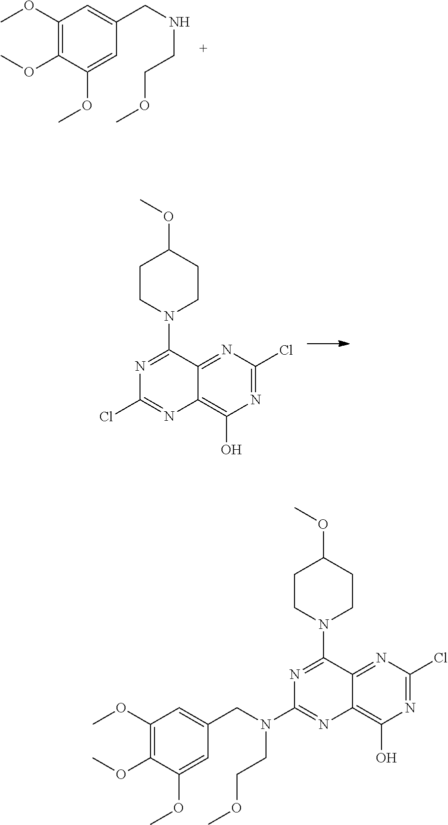 [SMILES: COCCNCc1cc(OC)c(OC)c(OC)c1 ; COC1CCN(c2nc(Cl)nc3c(O)nc(Cl)nc23)CC1 ; COCCN(Cc1cc(OC)c(OC)c(OC)c1)c1nc(N2CCC(OC)CC2)c2nc(Cl)nc(O)c2n1]

To a solution of intermediate compound 49 (1.29 g, 3.92 mmol, 1 eq) in NMP (10 mL) was added the intermediate compound 18 (1 g, 3.92 mmol, 1 eq) and DIPEA (1.11 g, 8.62 mmol, 1.50 mL, 2.2 eq). The mixture was stirred at 115° C. for 12 h. The residue was purified by prep-HPLC (column: Phenomenex luna C18 150*25 mm*10 um; mobile phase: [water(0.225% FA)-ACN];B %: 13%-43%, 10 min) to give the intermediate compound 78 (780 mg, 36% yield) as red solid.

LCMS (ESI position ion) m/z: (M+H)+: 549.2 (calculated: 548.21)

Intermediate Compound 79:

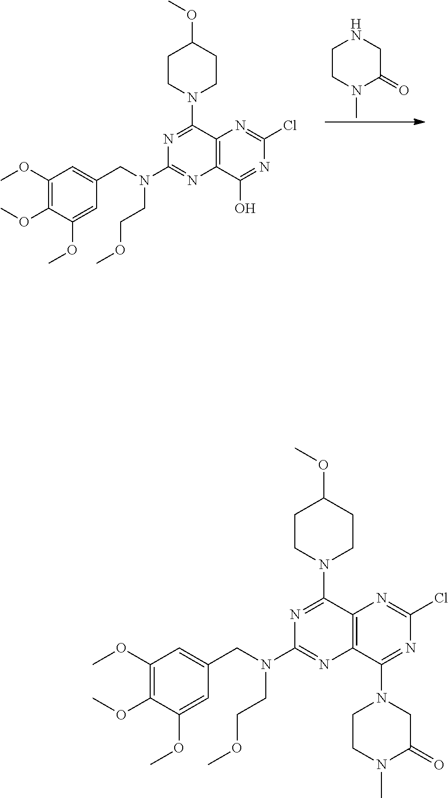

To a solution of intermediate compound 78 (180 mg, 327.86 umol, 1 eq) in DMF (5 mL) was added DIPEA (63.56 mg, 491.79 umol, 85.66 uL, 1.5 eq) and BOP (188.51 mg, 426.22 umol, 1.3 eq) at 0° C. and stirred 0.5 h. Then the 1-methylpiperazin-2-one (112.27 mg, 983.57 umol, 3 eq) was added and the mixture was stirred at 20° C. for 2.5 h. The reaction mixture was concentrated under vacuum. The residue was purified by prep-HPLC (column: Phenomenex luna C18 150*25 mm*10 um; mobile phase: [water(0.225% FA)-ACN];B %: 13%-43%, 10 min) to give the intermediate compound 79 (180 mg, 85% yield) as a yellow solid.

LCMS (ESI position ion) m/z: (M+H)+: 645.4 (calculated: 644.28)

Intermediate Compound 80:

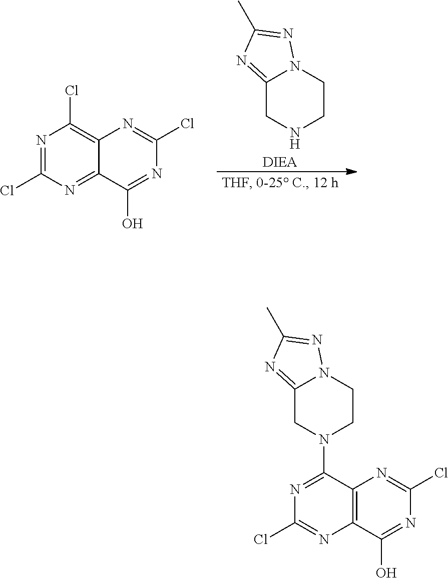

To a solution of intermediate compound 21 (400 mg, 1.59 mmol, 1 eq) in THF (5 mL) was added DIEA (513.98 mg, 3.98 mmol, 692.69 uL, 2.5 eq) and cooled to 0° C. A solution of 2-methyl-5,6,7,8-tetrahydro-[1,2,4]triazolo[1,5-a]pyrazine (219.79 mg, 1.59 mmol, 1 eq) in THF (5 mL) was drop wise added to the reaction mixture at 0° C. After addition, the mixture was stirred at 25° C. for 12 hr. The reaction mixture was concentrated under vacuum. This residue was diluted with aqueous citric acid (1.82 g/50 mL) and this suspension was stirred at 20° C. for 1 hr. Then the suspension was filtered, and the filter cake was washed by water (20.0 mL×3) and then dried under vacuum to give the intermediate compound 80 (600 mg, crude) as yellow solid.

LCMS (ESI position ion) m/z: (M+H)+: 352.8 (calculated: 352.03)

1H NMR (400 MHz, DMSO-d6) δ ppm 5.38 (br s, 2H), 4.61 (br s, 2H), 4.24 (br t, J=5.2 Hz, 2H), 2.23 (s, 3H)

Intermediate Compound 81:

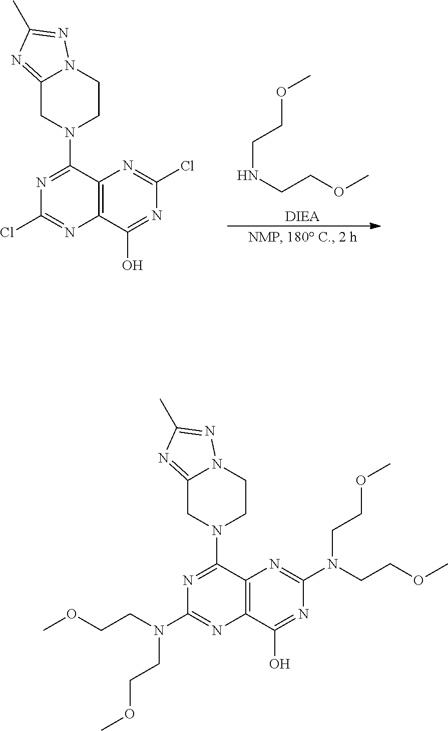

Intermediate compound 80 (180 mg, 509.68 umol, 1 eq), bis(2-methoxyethyl)amine (678.83 mg, 5.10 mmol, 752.58 uL, 10 eq) and DIEA (658.72 mg, 5.10 mmol, 887.76 uL, 10 eq) were taken up into a microwave tube in NMP (2 mL). The sealed tube was heated at 180° C. for 2 hr under microwave. The reaction mixture was filtered. The filtrate was purified by prep-HPLC (column: Phenomenex luna C18 150*25 mm*10 um; mobile phase: [water(0.225% FA)-ACN];B %: 13%-43%, 10 min) to give the intermediate compound 81 (260 mg, 93% yield) as yellow solid.

LCMS (ESI position ion) m/z: (M+H)+: 547.2 (calculated: 546.30)

1H NMR (400 MHz, CHLOROFORM-d) δ ppm 5.23 (s, 2H), 4.52 (br t, J=5.0 Hz, 2H), 4.34-4.26 (m, 2H), 3.88 (br t, J=5.6 Hz, 4H), 3.70-3.58 (m, 12H), 3.42 (s, 6H), 3.36 (s, 6H), 2.39 (s, 3H)

Intermediate Compounds 82 and 83:

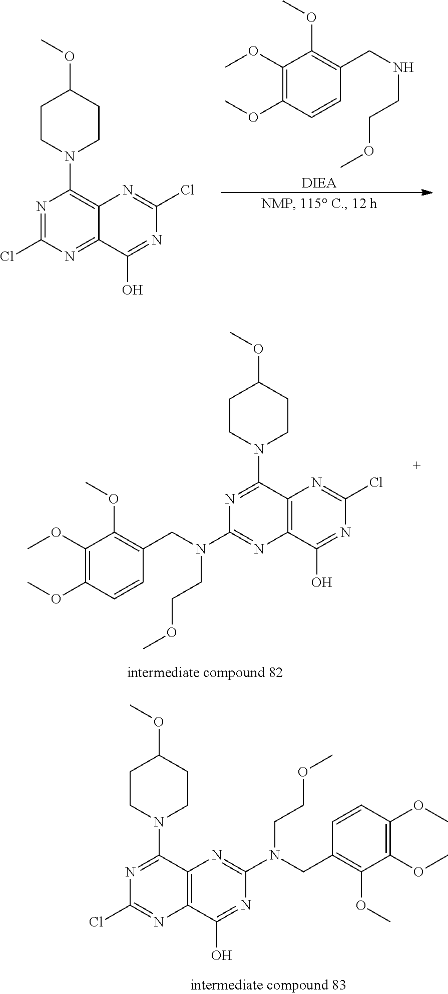

To a solution of intermediate compound 49 (6.5 g, 19.69 mmol, 1 eq) in NMP (20 mL) was added intermediate compound 17 (5.03 g, 19.69 mmol, 1 eq) and DIPEA (5.60 g, 43.31 mmol, 7.54 mL, 2.2 eq). The mixture was stirred at 115° C. for 12 h. The reaction mixture was concentrated under vacuum. The residue was purified by prep-HPLC (column: Phenomenex luna C18 150*25 mm*10 um; mobile phase: [water(0.225% FA)-ACN];B %: 13%-43%, 10 min) to give the intermediate compound 82 (1.2 g, 11% yield) as red solid and the intermediate compound 83 (3.8 g, 35% yield) as yellow solid.

Intermediate Compound 82:

LCMS (ESI position ion) Rt=0.878, m/z: (M+H)+: 549.3 (calculated: 548.21)

Intermediate Compound 83:

LCMS (ESI position ion) Rt=0.951, m/z: (M+H)+: 549.3 (calculated: 548.21)

Intermediate Compound 84:

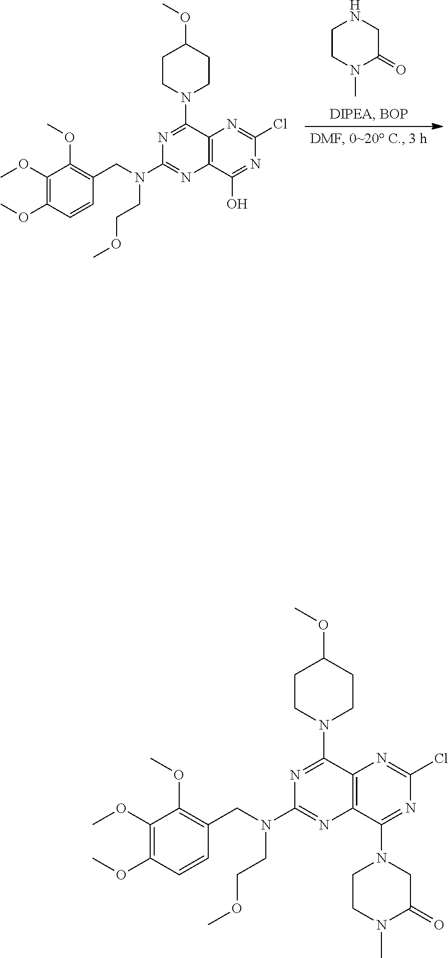

To a solution of intermediate compound 82 (500 mg, 910.72 umol, 1 eq) in DMF (5 mL) was added DIPEA (176.56 mg, 1.37 mmol, 237.95 uL, 1.5 eq) and BOP (523.63 mg, 1.18 mmol, 1.3 eq) at 0° C. and stirred 0.5 h. Then 1-methylpiperazin-2-one (311.86 mg, 2.73 mmol, 3 eq) was added to the mixture and the mixture stirred at 20° C. for 2.5 h. The reaction mixture was concentrated under vacuum. The residue was purified by prep-HPLC (column: Phenomenex luna C18 150*25 mm*10 um; mobile phase: [water(0.225% FA)-ACN];B %: 13%-43%, 10 min) to give the intermediate compound 84 (400 mg, 68% yield) as a yellow solid.

LCMS (ESI position ion) Rt=1.037, m/z: (M+H)+: 645.2 (calculated: 644.28)

Intermediate Compound 85:

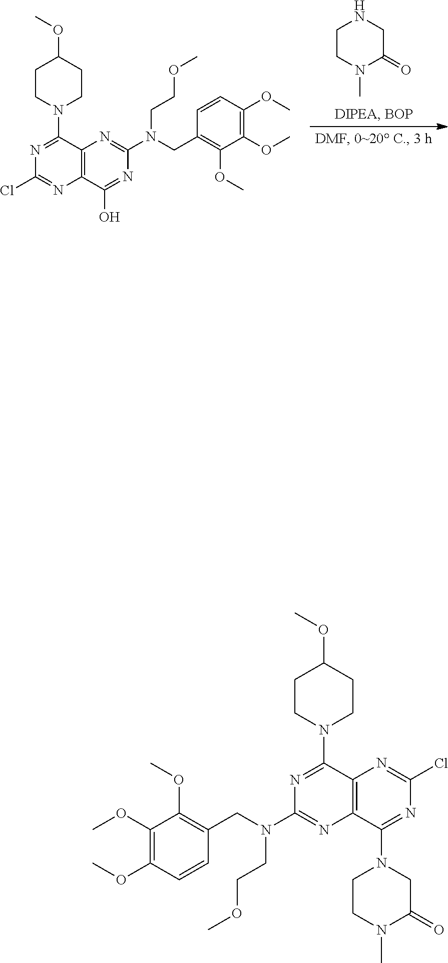

To a solution of intermediate compound 83 (500 mg, 910.72 umol, 1 eq) in DMF (1 mL) was added DIPEA (176.56 mg, 1.37 mmol, 237.95 uL, 1.5 eq) and BOP (523.63 mg, 1.18 mmol, 1.3 eq) at 0° C. and stirred 0.5 h. Then 1-methylpiperazin-2-one (311.86 mg, 2.73 mmol, 3 eq) was added to the mixture and the mixture was stirred at 20° C. for 2.5 h. The reaction mixture was concentrated under vacuum. The residue was purified by prep-HPLC (column: Phenomenex luna C18 150*25 mm*10 um; mobile phase: [water(0.225% FA)-ACN];B %: 13%-43%, 10 min) to give the intermediate compound 85 (450 mg, 76% yield) as a yellow solid.

LCMS (ESI position ion) Rt=1.031, m/z: (M+H)+: 645.2 (calculated: 644.28)

Intermediate Compound 86:

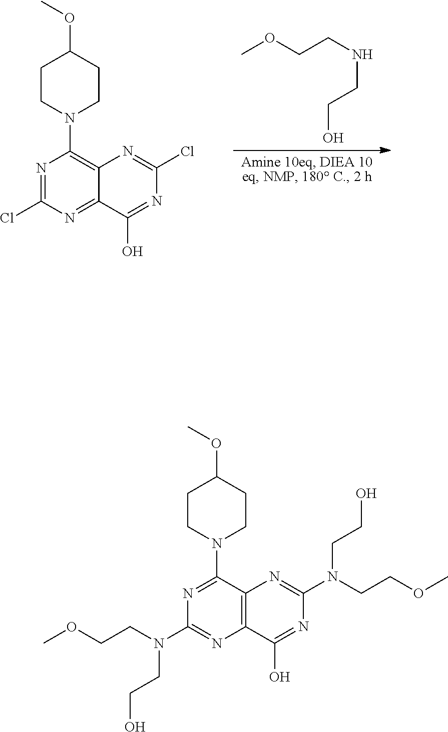

Intermediate compound 49 (150 mg, 454.31 umol, 1 eq), 2-((2-methoxyethyl)amino)ethan-1-ol (541.37 mg, 4.54 mmol, 10 eq) and DIEA (587.17 mg, 4.54 mmol, 791.33 uL, 10 eq) were taken up into a microwave tube in NMP (1.5 mL). The sealed tube was heated at 180° C. for 2 hr under microwave. The reaction mixture was filtered. The filtrate was purified by prep-HPLC (column: Phenomenex luna C18 150*25 mm*10 um; mobile phase: [water(0.05% FA)-ACN];B %: 13%-43%, 10 min) to give the intermediate compound 86 (180 mg, crude) as yellow solid.

LCMS (ESI position ion) m/z: (M+H)+: 496.2 (calculated: 495.28)

Intermediate Compound 87:

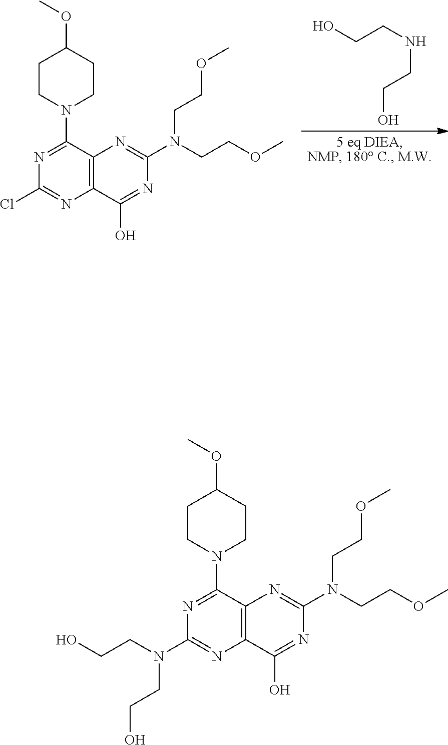

Intermediate compound 51 (1.00 g, 2.34 mmol, 1 eq), 2,2′-azanediylbis(ethan-1-ol) (2.46 g, 23.42 mmol, 2.26 mL, 10 eq) and DIEA (1.51 g, 11.71 mmol, 2.04 mL, 5 eq) were taken up into a microwave tube in NMP (4 mL). The sealed tube was heated at 180° C. for 2 hr under microwave. The reaction mixture was filtered. The filtrate was purified by prep-HPLC (column: Phenomenex luna C18 150*25 mm*10 um; mobile phase: [water(0.05% FA)-ACN];B %: 13%-43%, 10 min) to give the intermediate compound 87 (530 mg, 46% yield) as a yellow solid.

LCMS (ESI position ion) tR=0.788 min, m/z, m/z: (M+H)+: 496.4 (calculated: 495.28)

1H NMR (400 MHz, CHLOROFORM-d) δ ppm 4.49 (br s, 2H), 3.91-3.84 (m, 4H), 3.80-3.74 (m, 4H), 3.71-3.59 (m, 10H), 3.49-3.45 (m, 1H), 3.43-3.36 (m, 9H), 2.04-1.95 (m, 2H), 1.74-1.63 (m, 2H)

Intermediate Compound 88:

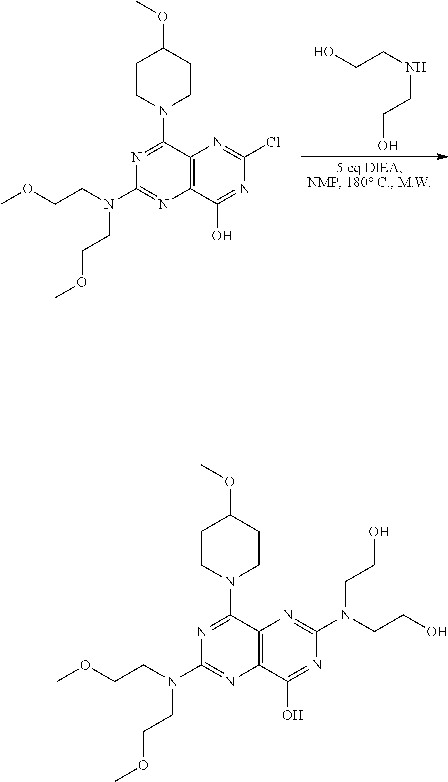

Intermediate compound 50 (1 g, 2.34 mmol, 1 eq), 2,2′-azanediylbis(ethan-1-ol) (2.46 g, 23.42 mmol, 2.26 mL, 10 eq) and DIEA (1.51 g, 11.71 mmol, 2.04 mL, 5 eq) were taken up into a microwave tube in NMP (4 mL). The sealed tube was heated at 180° C. for 2 hr under microwave. The reaction mixture was filtered. The filtrate was purified by prep-HPLC (column: Phenomenex luna C18 150*25 mm*10 um; mobile phase: [water(0.05% FA)-ACN];B %: 13%-43%, 10 min) to give the intermediate compound 88 (530 mg, 46% yield) as a yellow solid.

LCMS (ESI position ion) tR=0.787 min, m/z, m/z: (M+H)+: 496.3 (calculated: 495.28)

1H NMR (400 MHz, CHLOROFORM-d) δ ppm 4.40 (br s, 2H), 3.91-3.75 (m, 8H), 3.75-3.53 (m, 10H), 3.48 (td, J=4.0, 7.7 Hz, 1H), 3.39 (s, 3H), 3.36 (s, 6H), 2.04-1.94 (m, 2H), 1.75-1.62 (m, 2H).

Intermediate Compound 89:

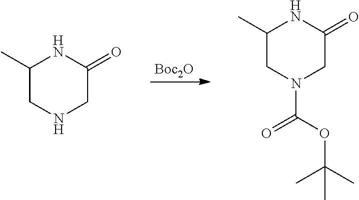

To a solution of 6-methylpiperazin-2-one (900 mg, 7.88 mmol, 1 eq) in DCM (9 mL) was added Boc2O (2.06 g, 9.46 mmol, 2.17 mL, 1.2 eq) and DIPEA (3.06 g, 23.65 mmol, 4.12 mL, 3 eq). The reaction was stirred at 25° C. for 12 hr. The reaction mixture was diluted with 100 ml water, extracted with dichloromethane (50 mL×2). The combined organic layers were washed with 50 mL citric acid solution and 50 mL brine, dried over Na2SO4 and filtered. The filtrate was concentrated under vacuum. The residue was purified by column chromatography (SiO2, Ethyl acetate, Rf=0.6) to give the tert-butyl 3-methyl-5-oxopiperazine-1-carboxylate (710 mg, 42% yield) was obtained as a white solid.

LCMS (ESI position ion) m/z: (M+H)+: 215.2 (calculated: 214.13)

1H NMR (400 MHz, CHLOROFORM-d) δ ppm 6.37-6.10 (m, 1H), 4.24 (d, J=18.5 Hz, 1H), 4.02-3.81 (m, 2H), 3.72-3.56 (m, 1H), 3.12-2.72 (m, 1H), 1.48 (s, 9H), 1.21 (d, J=6.5 Hz, 3H)

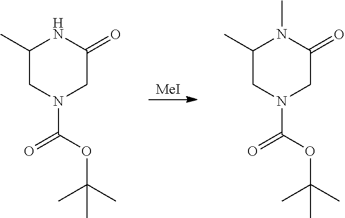 [SMILES: "MeI" ; CC1CN(C(=O)OC(C)(C)C)CC(=O)N1 ; CC1CN(C(=O)OC(C)(C)C)CC(=O)N1C]

To a solution of tert-butyl 3-methyl-5-oxopiperazine-1-carboxylate (710 mg, 3.31 mmol, 1 eq) in DMF (8.5 mL) was added NaH (198.80 mg, 4.97 mmol, 60% purity, 1.5 eq) with stirring at 0° C. for 0.5 h. And the mixture was added methyl iodide (705.52 mg, 4.97 mmol, 309.44 uL, 1.5 eq) with stirring at 0° C. Then the mixture was stirred at 25° C. for 3 h. The reaction mixture was diluted with 100 mL saturated ammonia chloride aqueous solution, extracted with Ethyl acetate (50 mL×2). The combined organic layers were washed with 100 mL brine, dried over Na2SO4 and filtered. The filtrate was concentrated under vacuum. The residue was purified by column chromatography (SiO2, Ethyl acetate, Rf=0.4) to give the tert-butyl 3,4-dimethyl-5-oxopiperazine-1-carboxylate (416 mg, 55% yield) as a colourless gum.

LCMS (ESI position ion) m/z: (M+H)+: 229.2 (calculated: 228.15)

1H NMR (400 MHz, METHANOL-d4) δ ppm 4.30-4.17 (m, 1H), 3.84 (br dd, J=1.4, 13.5 Hz, 2H), 3.61-3.50 (m, 1H), 3.46-3.34 (m, 1H), 2.98-2.93 (m, 3H), 1.48 (s, 9H), 1.25-1.23 (m, 3H).

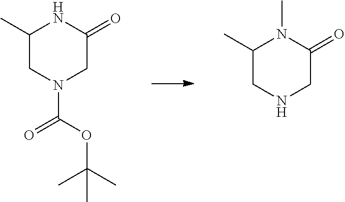

A mixture of tert-butyl 3,4-dimethyl-5-oxopiperazine-1-carboxylate (416 mg, 1.82 mmol, 1 eq) was added HCl/MeOH (4 M, 2 mL, 4.39 eq) with stirring at 20° C. for 2 h. The mixture was concentrated under vacuum giving the intermediate 89 (370 mg, 85% yield) as a brown gum used without further purification.

LCMS (ESI position ion) m/z: (M+H)+: 129.2 (calculated: 128.09)

1H NMR (400 MHz, METHANOL-d4) δ ppm 3.93-3.83 (m, 3H), 3.63 (dd, J=4.6, 13.1 Hz, 1H), 3.35-3.32 (m, 1H), 3.04-2.99 (m, 3H), 1.40 (d, J=6.6 Hz, 3H)

Intermediate Compound 90:

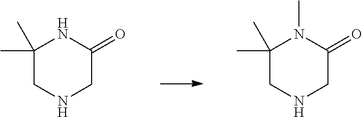

Following the protocols described for the intermediate compound 89 from 6,6-dimethylpiperazin-2-one, the intermediate compound 90 (142 mg, 68% yield) was obtained as a white solid.

LCMS (ESI position ion) m/z: (M+H)+: 143.2 (calculated: 142.11)

1H NMR (400 MHz, METHANOL-d4) ppm δ ppm 3.85 (s, 2H), 3.45 (s, 2H), 2.96 (s, 3H), 1.46 (s, 6H).

Intermediate Compound 91:

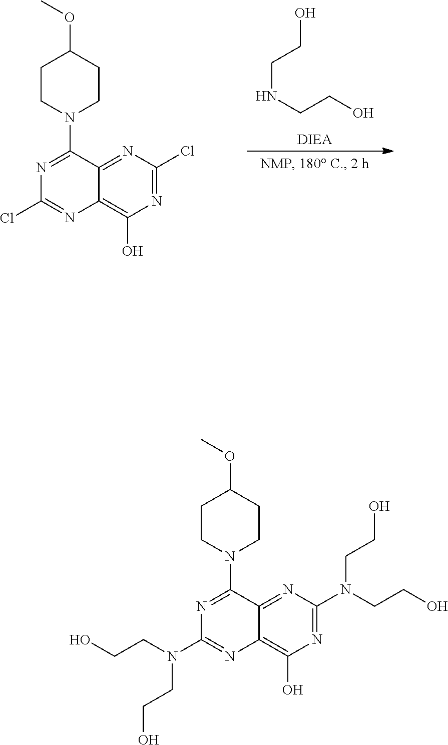

To a solution of intermediate compound 49 (480 mg, 1.45 mmol, 1 eq) in NMP (3 mL) was added DIPEA (1.88 g, 14.54 mmol, 2.53 mL, 10 eq) and 2,2′-azanediylbis(ethan-1-ol) (1.53 g, 14.54 mmol, 1.40 mL, 10 eq). The mixture was stirred at 180° C. for 2 h under microwave. The reaction mixture was concentrated under vacuum. The residue was purified by by prep-HPLC (column: Phenomenex luna C18 150*25 mm*10 um; mobile phase: [water(0.05% FA)-ACN];B %: 13%-43%, 10 min) to give the intermediate compound 91 (300 mg, 44% yield) as a yellow solid.

LCMS (ESI position ion) m/z: (M+H)+: 468.2 (calculated: 467.25)

Intermediate Compound 92:

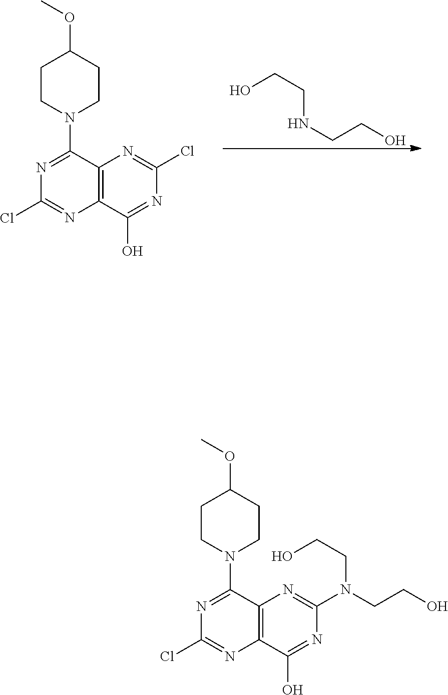 [SMILES: COC1CCN(c2nc(Cl)nc3c(O)nc(Cl)nc23)CC1 ; OCCNCCO ; COC1CCN(c2nc(Cl)nc3c(O)nc(N(CCO)CCO)nc23)CC1]

To a mixture of intermediate compound 49 (2 g, 6.06 mmol, 1 eq) and 2,2′-azanediylbis(ethan-1-ol) (636.86 mg, 6.06 mmol, 1 eq) in NMP (20 mL) was added DIEA (1.72 g, 13.33 mmol, 2.32 mL, 2.2 eq), and the mixture was stirred at 115° C. for 16 h. The mixture was purified by prep-HPLC(Column: Phenomenex luna C18 150*40 mm*15 um; Mobile phase: [water (0.05% FA)-ACN];B %: 15%-45%, 10 min; Wavelength: 220&254 nm) to give the intermediate compound 92 (900 mg, 37% yield) as a light yellow solid.

LCMS (ESI position ion) m/z: (M+H)+: 399.2 (calculated: 398.15)

1H NMR (400 MHz, DMSO-d6) δ ppm 1.61 (br d, J=5.75 Hz, 2H) 1.86-2.02 (m, 2H) 3.28 (s, 3H) 3.33 (s, 4H) 3.55-3.81 (m, 9H) 4.40-4.58 (m, 1H) 4.87-5.35 (m, 1H) 11.12-12.23 (m, 1H)

Intermediate Compound 93:

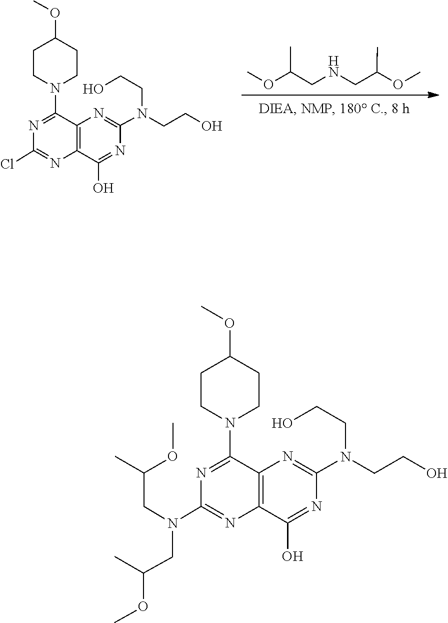

A mixture of intermediate compound 92 (495.69 mg, 2.51 mmol, 10 eq, HCl) and DIEA (648.09 mg, 5.01 mmol, 873.43 uL, 20 eq) in NMP (0.5 mL) was stirred at 10° C. for 0.5 h, bis(2-methoxypropyl)amine (100 mg, 250.72 umol, 1 eq) was added, then the mixture was stirred at 180° C. for 8 h under microwave. The mixture was purified by prep-HPLC(Column: Phenomenex Synergi C18 150*25 mm*10 um; Mobile phase: [water (0.1% TFA)-ACN];B %: 22%-52%, 10 min) Wavelength: 220&254 nm) to give the intermediate compound 93 (70 mg, crude) as a brown oil.

LCMS (ESI position ion) m/z: (M+H)+: 524.6 (calculated: 523.31)

Intermediate Compound 94:

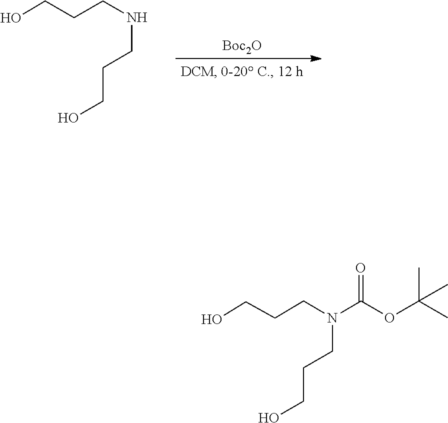 [SMILES: "Boc2O" ; OCCCNCCCO ; CC(C)(C)OC(=O)N(CCCO)CCCO]

To a solution of 3,3′-azanediylbis(propan-1-ol) (3 g, 22.52 mmol, 1 eq) in DCM (15 mL) was cooled to 0° C., a solution of Boc2O (4.92 g, 22.52 mmol, 5.17 mL, 1 eq) in DCM (15 mL) was added dropwise to the mixture at 0° C. The mixture was slowly warmed to 20° C. and stirred for 12 hr. The solvent was removed under vacuum to give the tert-butyl bis(3-hydroxypropyl)carbamate (5.3 g, crude) as a red oil.

1H NMR (400 MHz, CHLOROFORM-d) δ ppm 3.80 (br s, 1H), 3.71-3.52 (m, 4H), 3.45-3.22 (m, 4H), 1.82-1.70 (m, 4H), 1.48 (s, 9H).

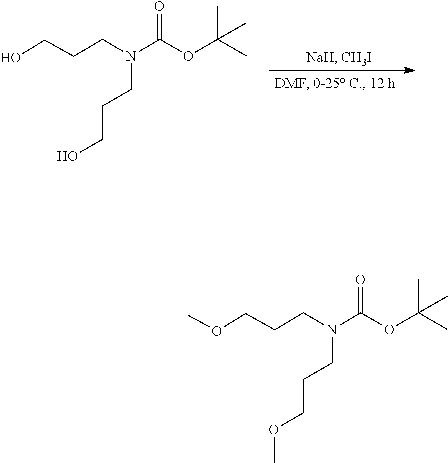

A mixture of NaH (857.16 mg, 21.43 mmol, 60% purity, 2.5 eq) in DMF (15 mL) was cooled to 0° C. A solution of tert-butyl bis(3-hydroxypropyl)carbamate (2 g, 8.57 mmol, 1 eq) in DMF (10 mL) was dropwise added to the mixture. The mixture was stirred at 0° C. for 30 min. After 30 min, a solution of iodomethane (3.65 g, 25.72 mmol, 1.60 mL, 3 eq) in DMF (5 mL) was dropwise added to the reaction mixture. The mixture was warmed to 25° C. and stirred for 12 hr. The reaction mixture was slowly poured into saturated aqueous NH4Cl at 0° C. Then the mixture was extracted with Ethyl acetate (80 mL×3). The organic layer was washed with brine, dried by Na2SO4 and concentrated. The residue was purified by column chromatography (SiO2, Petroleum ether/Ethyl acetate=8/1 to 3/1) to give the tert-butyl bis(3-methoxypropyl)carbamate (Rf=0.73) (1.81 g, 80% yield) as yellow liquid.

1H NMR (400 MHz, CHLOROFORM-d) δ ppm 3.38 (t, J=6.3 Hz, 4H), 3.32 (s, 6H), 3.25 (br s, 4H), 1.86-1.75 (m, 4H), 1.46 (s, 9H).

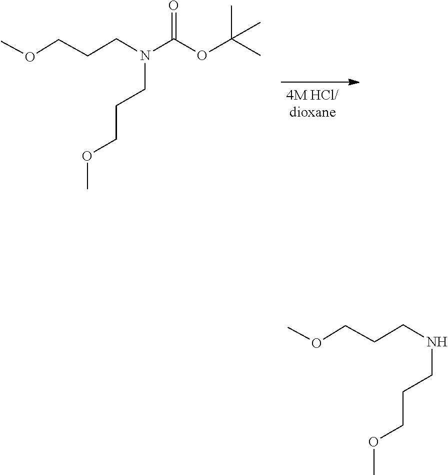

To a solution of tert-butyl bis(3-methoxypropyl)carbamate (1.81 g, 6.93 mmol, 1 eq) in dioxane (10 mL) was added HCl/dioxane (4 M, 20 mL, 11.55 eq) at 0° C. The mixture was stirred at 20° C. for 3 hr. The solvent was removed under vacuum to give the intermediate compound 94 (1.37 g, crude) as off white solid.

1H NMR (400 MHz, DMSO-d6) δ ppm 9.02 (br s, 2H), 3.38 (t, J=6.1 Hz, 4H), 3.23 (s, 6H), 2.89 (br d, J=2.2 Hz, 4H), 1.94-1.81 (m, 4H).

Intermediate Compound 95:

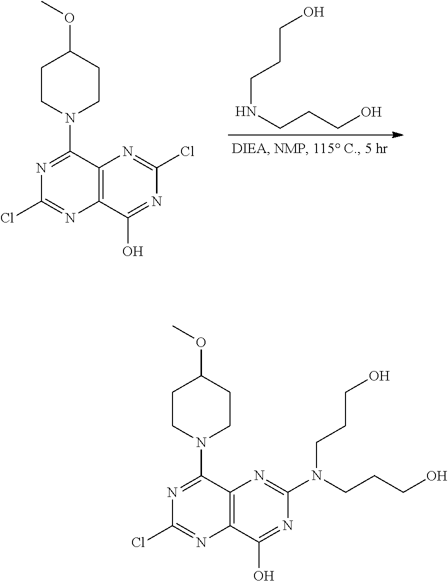 [SMILES: COC1CCN(c2nc(Cl)nc3c(O)nc(Cl)nc23)CC1 ; OCCCNCCCO ; COC1CCN(c2nc(Cl)nc3c(O)nc(N(CCCO)CCCO)nc23)CC1]

To a solution of intermediate compound 49 (2 g, 6.06 mmol, 1 eq) in NMP (8 mL) were added DIEA (1.72 g, 13.33 mmol, 2.32 mL, 2.2 eq) and 3,3′-azanediylbis(propan-1-ol) (806.79 mg, 6.06 mmol, 1 eq). The mixture was stirred at 115° C. for 5 hr. The reaction mixture was filtered. The filtrate was purified by prep-HPLC (column: Phenomenex luna C18 150*40 mm*15 um; mobile phase: [water(0.1% TFA)-ACN];B %: 16%-46%, 11 min) to give the intermediate compound 95 (460 mg, 18% yield) as yellow solid.

LCMS (ESI position ion) m/z: (M+H)+: 427.1 (calculated: 426.18)

1H NMR (400 MHz, DMSO-d6) δ ppm 11.54 (br s, 1H), 4.56 (br s, 2H), 3.75 (br s, 1H), 3.53-3.39 (m, 12H), 3.28 (s, 3H), 1.93 (br s, 2H), 1.77-1.65 (m, 4H), 1.59-1.47 (m, 2H).

Intermediate Compound 96:

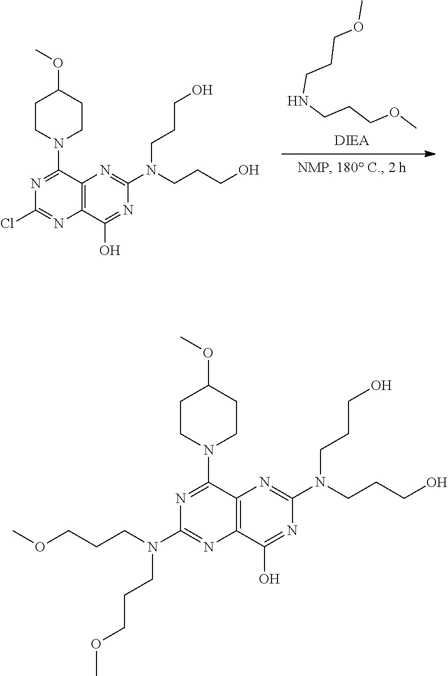 [SMILES: COC1CCN(c2nc(Cl)nc3c(O)nc(N(CCCO)CCCO)nc23)CC1 ; COCCCNCCCOC ; COCCCN(CCCOC)c1nc(N2CCC(OC)CC2)c2nc(N(CCCO)CCCO)nc(O)c2n1]

Intermediate compound 95 (220 mg, 515.35 umol, 1 eq), intermediate compound 94 (1.22 g, 6.18 mmol, 12 eq, HCl) and DIEA (1.13 g, 8.76 mmol, 1.53 mL, 17 eq) were taken up into a microwave tube in NMP (2 mL). The sealed tube was heated at 180° C. for 2 hr under microwave. The reaction mixture was filtered. The filtrate was purified by by prep-HPLC (column: Phenomenex luna C18 150*25 mm*10 um; mobile phase: [water(0.05% FA)-ACN];B %: 13%-43%, 10 min) to give the intermediate compound 96 (187 mg, 66% yield) as yellow solid.

LCMS (ESI position ion) m/z: (M+H)+: 552.4 (calculated: 551.34)

Intermediate Compound 97:

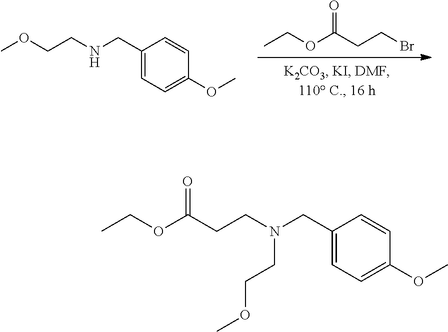

A mixture of 2-methoxy-N-(4-methoxybenzyl)ethan-1-amine (1 g, 5.12 mmol, 1 eq) and ethyl 3-bromopropanoate (1.04 g, 5.74 mmol, 731.25 uL, 1.12 eq) in DMF (10 mL) was added K2CO3 (1.56 g, 11.27 mmol, 2.2 eq) and KI (850.17 mg, 5.12 mmol, 1 eq). The mixture was stirred at 90° C. for 16 h, then at 110° C. for 16 h. To the reaction mixture was added water (50 mL) and extracted with ethyl acetate (80 mL×3). The organic layers were dried over anhydrous Na2SO4 and concentrated under vacuum. The residue was purified by column chromatography (SiO2, Petroleum ether/Ethyl acetate=10/1 to 4/1) to give the ethyl 3-((4-methoxybenzyl)(2-methoxyethyl)amino)propanoate (850 mg, 56% yield) as colourless oil.

LCMS (ESI position ion) m/z: (M+H)+: 296.4 (calculated: 295.18)

1H NMR (400 MHz, CHLOROFORM-d) δ ppm 1.25 (t, J=7.15 Hz, 3H) 2.48 (t, J=7.21 Hz, 2H) 2.61-2.69 (m, 2H) 2.87 (t, J=7.21 Hz, 2H) 3.31 (s, 3H) 3.41-3.48 (m, 2H) 3.54-3.63 (m, 2H) 3.80 (s, 3H) 4.05-4.18 (m, 2H) 6.80-6.89 (m, 2H) 7.18-7.26 (m, 2H)

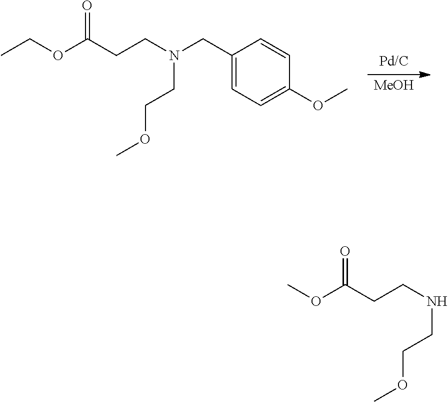

A mixture of ethyl 3-((4-methoxybenzyl)(2-methoxyethyl)amino)propanoate (850 mg, 2.88 mmol, 1 eq) in MeOH (20 mL) was degassed with Ar, the mixture was added wet Pd/C (500 mg, 1.44 mmol, 10% purity, 0.5 eq), the mixture was stirred under H2 for 24 h at 30° C. The mixture was filtered and the filtrate was concentrated under vacuum to give the intermediate compound 97 (750 mg, crude) as colourless oil.

LCMS (ESI position ion) m/z: (M+H)+: 162.2 (calculated: 161.10)

1H NMR (400 MHz, CHLOROFORM-d) δ ppm 2.37-2.43 (m, 2H) 2.65 (t, J=5.14 Hz, 2H) 2.74-2.79 (m, 2H) 3.23 (s, 3H) 3.36 (t, J=5.20 Hz, 2H) 3.56 (s, 3H) 6.63-6.69 (m, 1H) 6.93-6.96 (m, 1H)

Intermediate Compound 98:

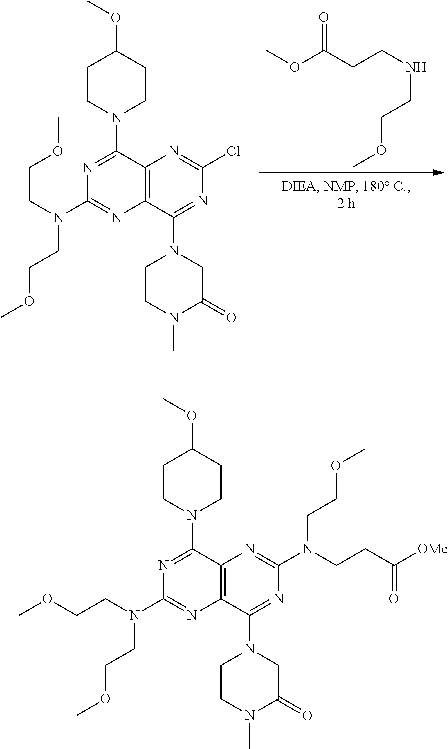 [SMILES: COCCN(CCOC)c1nc(N2CCC(OC)CC2)c2nc(Cl)nc(N3CCN(C)C(=O)C3)c2n1 ; COCCNCCC(=O)OC ; COCCN(CCC(=O)OC)c1nc(N2CCN(C)C(=O)C2)c2nc(N(CCOC)CCOC)nc(N3CCC(OC)CC3)c2n1]

A mixture of intermediate compound 52 (150 mg, 286.79 umol, 1 eq) in NMP (3 mL) was added intermediate compound 97 (693.46 mg, 4.30 mmol, 15 eq) and DIEA (555.99 mg, 4.30 mmol, 749.31 uL, 15 eq), the mixture was heated to 180° C. and stirred for 2 h under microwave. The reaction mixture was filtered. The filtrate was purified by prep-HPLC (column: Waters Xbridge 150*25 mm*5 um; mobile phase: [water(10 mM NH4HCO3)-ACN]; B %: 48%-72%, 7 min) to give the intermediate compound 98 (60 mg, crude) as yellow oil.

LCMS (ESI position ion) m/z: (M+H)+: 648.4 (calculated: 647.37)

Intermediate Compound 99:

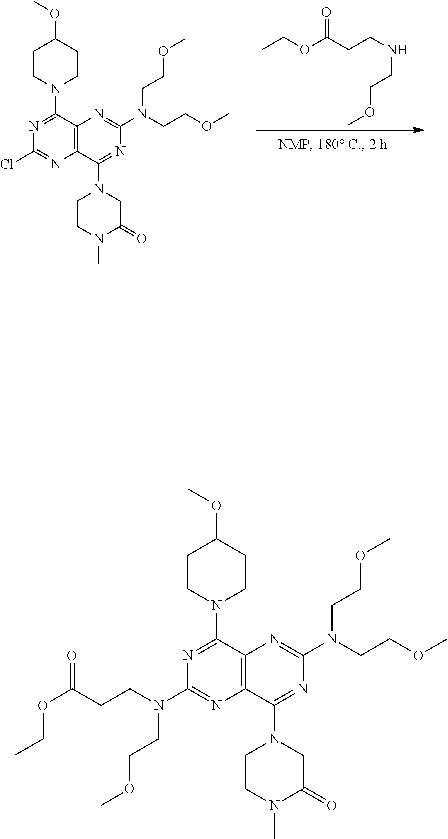 [SMILES: COCCN(CCOC)c1nc(N2CCN(C)C(=O)C2)c2nc(Cl)nc(N3CCC(OC)CC3)c2n1 ; CCOC(=O)CCNCCOC ; CCOC(=O)CCN(CCOC)c1nc(N2CCC(OC)CC2)c2nc(N(CCOC)CCOC)nc(N3CCN(C)C(=O)C3)c2n1]

Intermediate compound 53 (160 mg, 305.91 umol, 1 eq), ethyl 3-((2-methoxyethyl)amino)propanoate (following the intermediate compound 97 protocol by using ethanol at the p-methoxybenzyl deprotection step) (804.05 mg, 4.59 mmol, 15 eq) and DIEA (593.05 mg, 4.59 mmol, 799.26 uL, 15 eq) were taken up into a microwave tube in NMP (3 mL). The sealed tube was heated at 180° C. for 1.5 hr under microwave. The reaction mixture was filtered. The filtrate was purified by prep-HPLC (column: Waters Xbridge 150*25 mm*5 um; mobile phase: [water(10 mM NH4HCO3)-ACN]; B %: 42%-75%, 9 min) to give the intermediate compound 99 (60 mg, 30% yield) as yellow gum.

LCMS (ESI position ion) m/z: (M+H)+: 662.4 (calculated: 661.39)

Intermediate Compound 100:

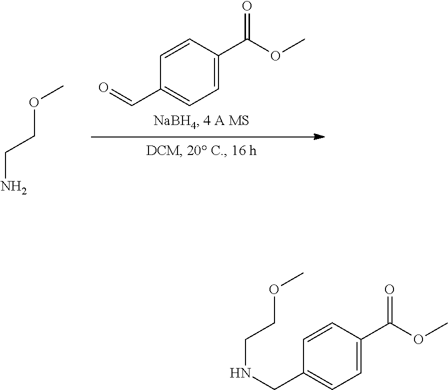

To a solution of 2-methoxyethan-1-amine (2 g, 26.63 mmol, 2.31 mL, 1 eq) and methyl 4-formylbenzoate (4.37 g, 26.63 mmol, 1 eq) in DCM (20 mL) was added 4A° MS (2 g, 26.63 mmol, 1 eq) in one portion. This reaction mixture was stirred at 20° C. for 12 hr. To the mixture was added methanol (5 mL) and cooled to 0° C., NaBH4 (1.11 g, 29.29 mmol, 1.1 eq) was added batch wise at 0° C., the mixture was stirred at 20° C. for 2 hr. The reaction mixture was quenched by addition of ice water (100 mL) at 0° C., and then added 1N HCl adjust to pH=2 and stirred 0.5 h. the reaction mixture was diluted with ethyl acetate (300 mL) and washed with water (200 mL×3). The combined aqueous layers were extracted with ethyl acetate (100 mL×3), then to the aqueous layer was added 1N NaOH adjust to the pH=12 and diluted with ethyl acetate (500 mL) and washed with water (300 mL×3). The combined organic layers were washed with water (100 mL *3) dried over Na2SO4, filtered and concentrated under vacuum. The residue was purified by reversed phase (basic condition) to give the intermediate compound 100 (7 g, crude) as red oil.

1H NMR (400 MHz, CHLOROFORM-d) δ ppm 7.99 (d, J=8.3 Hz, 2H), 7.40 (d, J=8.3 Hz, 2H), 3.90 (s, 3H), 3.86 (s, 2H), 3.53-3.48 (m, 2H), 3.35 (s, 3H), 2.83-2.75 (m, 2H).

Intermediate Compound 101:

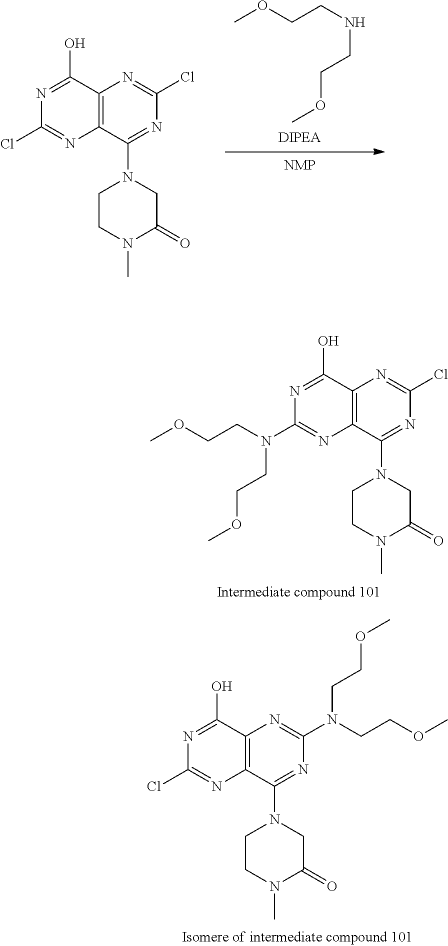

To a solution of intermediate compound 22 (1.12 g, 3.40 mmol, 1 eq) in NMP (4 mL) were added DIEA (967.51 mg, 7.49 mmol, 1.30 mL, 2.2 eq) and bis(2-methoxyethyl)amine (453.21 mg, 3.40 mmol, 502.45 uL, 1 eq). The mixture was stirred at 115° C. for 5 hr. The reaction mixture was filtered. The filtrate was purified by Prep-HPLC (column: Phenomenex luna C18 150*40 mm*15 um; mobile phase: [water (0.05% FA)-ACN]; B %: 14-44%, 10 min) to give intermediate compound 101 (640 mg, 44% yield) as an-off white solid and the isomer of intermediate compound 101 (180 mg, crude).

Intermediate Compound 101:

LCMS (ESI position ion) 0.718 min, m/z: (M+H)+: 426.3 (calculated: 425.16)

Isomere of the Intermediate Compound 101:

LCMS (ESI position ion) 0.657 min, m/z: (M+H)+: 426.3 (calculated: 425.16)

Intermediate Compound 102:

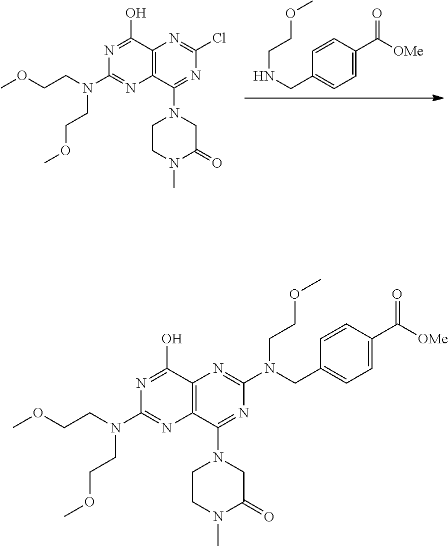 [SMILES: COCCN(CCOC)c1nc(O)c2nc(Cl)nc(N3CCN(C)C(=O)C3)c2n1 ; COCCNCc1ccc(C(=O)OC)cc1 ; COCCN(Cc1ccc(C(=O)OC)cc1)c1nc(N2CCN(C)C(=O)C2)c2nc(N(CCOC)CCOC)nc(O)c2n1]

A mixture of intermediate compound 101 (150 mg, 352.22 umol, 1 eq) and intermediate compound 100 (786.40 mg, 3.52 mmol, 10 eq) was stirred at 130° C. for 16 hr. The mixture was diluted with MeCN (3 mL) and filtered. The filtrate was purified by prep-HPLC (column: Phenomenex luna C18 150*40 mm*15 um; mobile phase: [water (0.05% FA)-ACN]; B %: 15-45%, 10 min) to give the intermediate compound 102 (100 mg, 37% yield, 79% purity) as a yellow solid.

LCMS (ESI position ion) m/z: (M+H)+: 613.4 (calculated: 612.30)

Intermediate Compound 103:

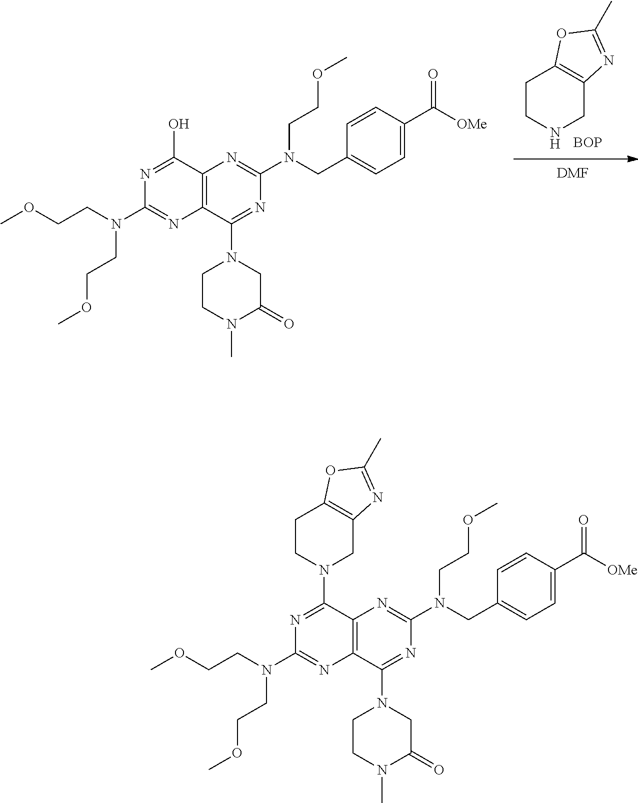

To a mixture of intermediate compound 102 (100 mg, 163.22 umol, 1 eq) and DIPEA (84.38 mg, 652.87 umol, 113.72 uL, 4 eq) in DMF (1 mL) was added BOP (86.63 mg, 195.86 umol, 1.2 eq) at 0° C. The mixture was stirred at 0° C. for 0.5 hr. Then 2-methyl-4,5,6,7-tetrahydrooxazolo[4,5-c]pyridine (42.75 mg, 244.83 umol, 1.5 eq, HCl salt) was added and the mixture was stirred at 20° C. for 1.5 hr. The mixture was filtered. The filtrate was purified by by prep-HPLC (column: Phenomenex luna C18 150*25 mm*10 um; mobile phase: [water(0.05% FA)-ACN];B %: 13%-43%, 10 min) to give the intermediate compound 103 (90 mg, 71% yield) as a yellow solid.

LCMS (ESI position ion) m/z: (M+H)+: 733.3 (calculated: 732.37)

1H NMR (400 MHz, CHLOROFORM-d) δ ppm 7.95 (d, J=7.6 Hz, 2H), 7.32-7.24 (m, 2H), 4.96 (s, 2H), 4.90-3.95 (m, 8H), 3.90 (s, 3H), 3.85-3.68 (m, 6H), 3.67-3.51 (m, 6H), 3.49-3.39 (m, 2H), 3.35 (d, J=0.4 Hz, 6H), 3.31 (s, 3H), 3.00 (s, 3H), 2.91-2.52 (m, 2H), 2.42 (s, 3H).

Intermediate Compound 104:

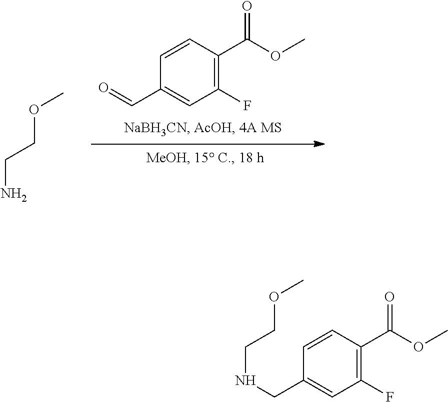

A mixture of 2-methoxyethan-1-amine (217.03 mg, 2.89 mmol, 251.19 uL, 1 eq), methyl 2-fluoro-4-formylbenzoate (500 mg, 2.75 mmol, 0.95 eq) and AcOH (173.52 mg, 2.89 mmol, 165.26 uL, 1 eq), 4A MS (500 mg) in MeOH (5 mL) was stirred at 15° C. for 2 hours. NaBH3CN (544.74 mg, 8.67 mmol, 3 eq) was added to the mixture, the mixture was stirred at 15° C. for 16 hours. The reaction mixture was filtered and concentrated under vacuum. The residue was purified by prep-HPLC (column: Phenomenex Gemini-NX C18 75×30 mm×3 um; mobile phase: [water (0.05% ammonia hydroxide v/v)-ACN];B %: 13%-43%, 7 min) to give the intermediate compound 104 (300 mg, 43% yield) as a colorless oil.

1H NMR (400 MHz, CHLOROFORM-d) δ ppm 7.92-7.86 (m, 1H), 7.20-7.13 (m, 2H), 3.92 (s, 3H), 3.86 (s, 2H), 3.55-3.48 (m, 2H), 3.37 (s, 3H), 2.83-2.75 (m, 2H).

Intermediate Compound 105:

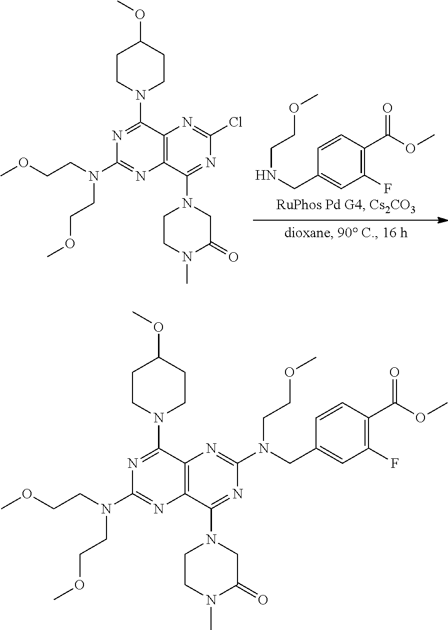

A mixture of intermediate compound 52 (150 mg, 286.79 umol, 1 eq), intermediate compound 104 (103.79 mg, 430.19 umol, 1.5 eq), RuPhos Pd G4 (24.39 mg, 28.68 umol, 0.1 eq), Cs2CO3 (280.33 mg, 860.38 umol, 3 eq) in dioxane (3 mL) was degassed and purged with N2 for three times, and then the mixture was stirred at 90° C. for 16 hours under N2 atmosphere. The reaction mixture was filtered and concentrated under vacuum. The residue was purified by prep-TLC (SiO2, EA:EtOH=10:1, Rf=0.38) to give the intermediate compound 105 (168 mg, 80% yield) as a yellow solid.

LCMS (ESI position ion) m/z: (M+H)+: 728.4 (calculated: 727.38)

Intermediate Compound 106:

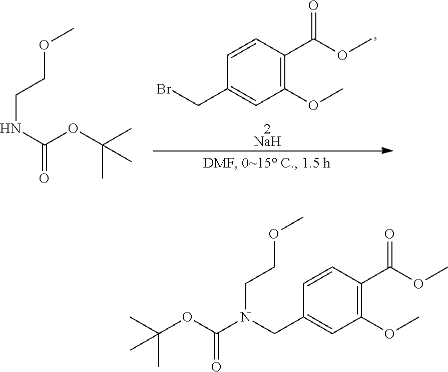

To a solution of tert-butyl (2-methoxyethyl)carbamate (500 mg, 2.85 mmol, 1 eq) in DMF (10 mL) was added NaH (171.19 mg, 4.28 mmol, 60% purity, 1.5 eq) at 0° C., the mixture was stirred at 0° C. for 0.5 h. Then methyl 4-(bromomethyl)-2-methoxybenzoate (813.26 mg, 3.14 mmol, 1.1 eq) was added to the mixture at 0° C. The resulting mixture was stirred at 15° C. for 1 h. The mixture was poured into ice-NH4C1 (sat., 20 mL). The mixture was extracted with Ethyl acetate (10 mL). The organic layer was washed with brine (20 mL), dried with anhydrous Na2SO4, filtered and concentrated in vacuum. The residue was purified by column chromatography (SiO2, Petroleum ether/Ethyl acetate=100/1 to 3/1, RF=0.3) to give the methyl 4-(((tert-butoxycarbonyl)(2-methoxyethyl)amino)methyl)-2-methoxybenzoate (3, 650 mg, 59% yield) as colorless oil.

LCMS (ESI position ion) m/z: (M+H)+: 254.2 (M-100) (calculated: 353.18)

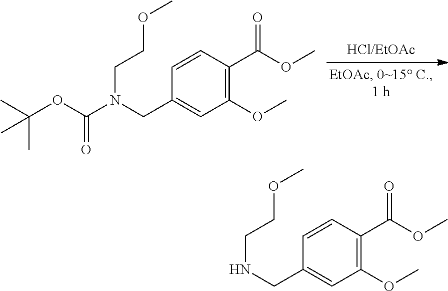 [SMILES: COCCN(Cc1ccc(C(=O)OC)c(OC)c1)C(=O)OC(C)(C)C ; COCCNCc1ccc(C(=O)OC)c(OC)c1]

To a solution of methyl 4-(((tert-butoxycarbonyl)(2-methoxyethyl)amino)methyl)-2-methoxybenzoate (650 mg, 1.84 mmol, 1 eq) in Ethyl acetate (2 mL) was added HCl/Ethyl acetate (4 M, 4 mL) at 0° C. The mixture was stirred at 15° C. for 1 h. The mixture was concentrated in vacuum. The residue was diluted with NaHCO₃ (sat. 10 mL) and extracted with Ethyl acetate (10 mL×5). The combined organic layer was dried with anhydrous Na₂SO₄, filtered and concentrated in vacuum to give the intermediate compound 106 (372 mg, 75% yield) as colorless oil.

LCMS (ESI position ion) m/z: (M+H)+: 253.8 (M-100) (calculated: 253.13)

1H NMR (400 MHz, CHLOROFORM-d) δ ppm 7.77 (d, J=7.8 Hz, 1H), 7.11-7.04 (m, 1H), 6.97-6.92 (m, 1H), 3.95-3.93 (m, 3H), 3.89 (s, 5H), 3.54 (br t, J=5.0 Hz, 2H), 3.37 (s, 3H), 2.85-2.80 (m, 2H).

Intermediate Compound 107:

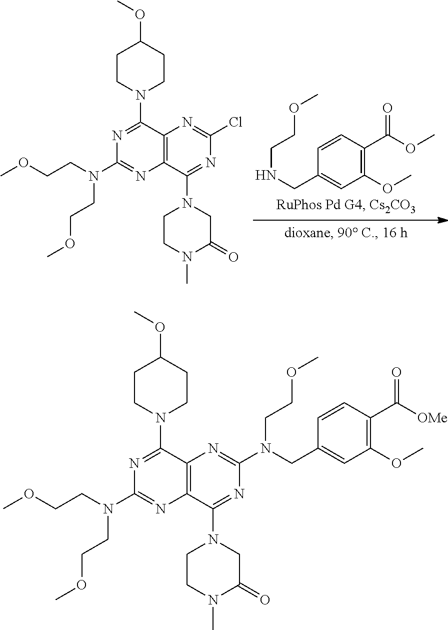 [SMILES: COCCN(CCOC)c1nc(N2CCC(OC)CC2)c2nc(Cl)nc(N3CCN(C)C(=O)C3)c2n1 ; COCCNCc1ccc(C(=O)OC)c(OC)c1 ; COCCN(Cc1ccc(C(=O)OC)c(OC)c1)c1nc(N2CCN(C)C(=O)C2)c2nc(N(CCOC)CCOC)nc(N3CCC(OC)CC3)c2n1]

To a mixture of intermediate compound 52 (50 mg, 95.60 umol, 1 eq), intermediate compound 106 (36.32 mg, 143.40 umol, 1.5 eq) in dioxane (3 mL) was added Cs2CO3 (93.44 mg, 286.79 umol, 3 eq) and Ruphos Pd G4 (8.13 mg, 9.56 umol, 0.1 eq) under N2. The mixture was stirred at 90° C. for 16 h. The mixture was filtered and concentrated in vacuum. The residue was purified by Prep-TLC (Ethyl acetate/Ethanol=10/1, RF=0.3) to give the intermediate compound 107 (40 mg, 56% yield) as a yellow solid.

LCMS (ESI position ion) m/z: (M+H)+: 740.4 (calculated: 739.40)

Intermediate Compound 108:

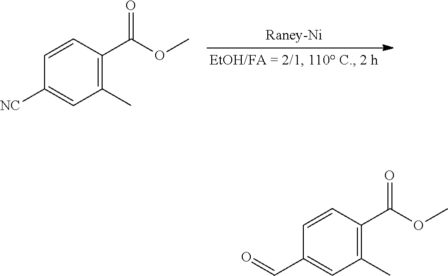

To a solution of methyl 4-cyano-2-methylbenzoate (3 g, 17.12 mmol, 1 eq) in EtOH (60 mL) were added Raney-Ni (washed with ethanol for 3 times) (3 g, 35.02 mmol, 2.04 eq) and HCOOH (30 mL) under N2. The suspension was degassed under vacuum and purged with N2 several times. The mixture was stirred at 110° C. for 2 hr. The reaction mixture was filtered. The filter cake was washed with DCM (50 mL×3). The filtrate was concentrated under vacuum. The residue was purified by column chromatography (SiO2, Petroleum ether/Ethyl acetate=50/1 to 10/1) to give the methyl 4-formyl-2-methylbenzoate (2.62 g, 86% yield) as white solid.

1H NMR (400 MHz, CHLOROFORM-d) δ ppm 10.05 (s, 1H), 8.03 (d, J=8.4 Hz, 1H), 7.78-7.72 (m, 2H), 3.94 (s, 3H), 2.67 (s, 3H)

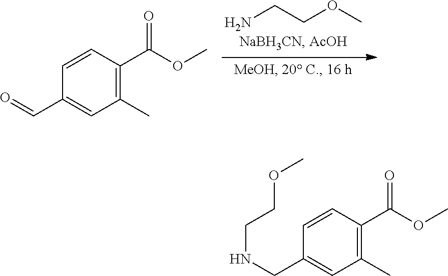

To a solution of methyl 4-formyl-2-methylbenzoate (1 g, 5.61 mmol, 1 eq) in MeOH (20 mL) were added AcOH (337.01 mg, 5.61 mmol, 320.96 uL, 1 eq), 4A MS (1 g) and 2-methoxyethan-1-amine (421.53 mg, 5.61 mmol, 487.88 uL, 1 eq) at 0° C. The mixture was stirred at 20° C. for 1 hr, then NaBH3CN (705.34 mg, 11.22 mmol, 2 eq) was added in portions at 20° C. and the mixture was stirred at 20° C. for 12 hr. The reaction mixture was filtered and concentrated under vacuum. The residue was purified by prep-HPLC (column: Waters Xbridge C18 150*50 mm*10 um; mobile phase: [water(10 mM NH4HCO3)-ACN];B %: 23%-53%, 11 min) to give the intermediate compound 108 (490 mg, 37% yield) as yellow oil.

LCMS (ESI position ion) m/z: (M+H)+: 238.2 (calculated: 237.14)

1H NMR (400 MHz, CHLOROFORM-d) δ ppm 7.89 (d, J=7.8 Hz, 1H), 7.25-7.18 (m, 2H), 3.89 (s, 3H), 3.83 (s, 2H), 3.55-3.50 (m, 2H), 3.37 (s, 3H), 2.81 (t, J=5.1 Hz, 2H), 2.60 (s, 3H)

Intermediate Compound 109:

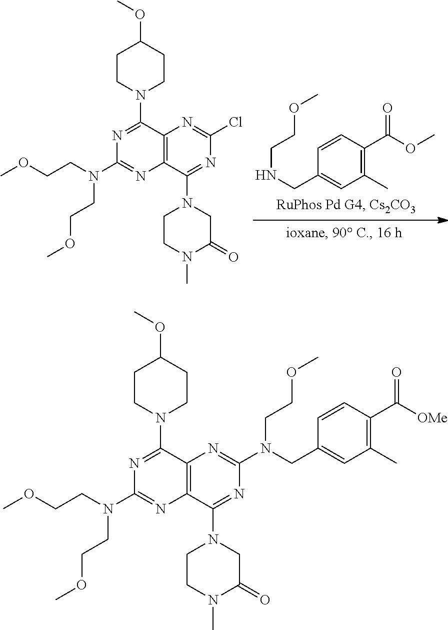 [SMILES: COCCN(CCOC)c1nc(N2CCC(OC)CC2)c2nc(Cl)nc(N3CCN(C)C(=O)C3)c2n1 ; COCCNCc1ccc(C(=O)OC)c(C)c1 ; COCCN(Cc1ccc(C(=O)OC)c(C)c1)c1nc(N2CCN(C)C(=O)C2)c2nc(N(CCOC)CCOC)nc(N3CCC(OC)CC3)c2n1]

To a mixture of intermediate compound 52 (100 mg, 191.19 umol, 1 eq), intermediate compound 108 (68.05 mg, 286.79 umol, 1.5 eq) in dioxane (2 mL) was added Cs2CO3 (186.88 mg, 573.58 umol, 3 eq) and Ruphos Pd G4 (32.52 mg, 38.24 umol, 0.2 eq) under N2. The mixture was stirred at 90° C. for 16 hr. The reaction mixture was filtered and concentrated under vacuum. The residue was purified by prep-TLC (SiO2, EA:EtOH=10:1) to give the intermediate compound 109 (100 mg, 72% yield) as yellow oil.

LCMS (ESI position ion) m/z: (M+H)+: 724.5 (calculated: 723.41)

Intermediate Compound 110:

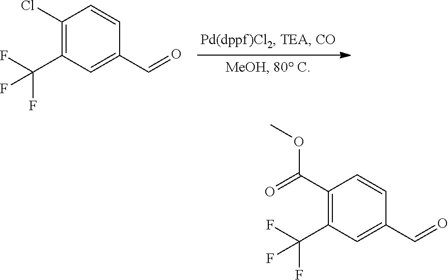 [SMILES: O=Cc1ccc(Cl)c(C(F)(F)F)c1 ; COC(=O)c1ccc(C=O)cc1C(F)(F)F]

A suspension of 4-chloro-3-(trifluoromethyl)benzaldehyde (3 g, 14.38 mmol, 1 eq), Pd(dppf)Cl2 (2.10 g, 2.88 mmol, 0.2 eq), TEA (4.37 g, 43.15 mmol, 6.01 mL, 3 eq) in MeOH (30 mL) was stirred at 80° C. for 24 hr under CO (50 psi) atmosphere. The reaction mixture was filtered and concentrated. The residue was purified by flash silica gel chromatography (ISCO®; 40 g SepaFlash® Silica Flash Column, Eluent of 3-10% Ethyl acetate/Petroleum ethergradient @ 70 mL/min) to give the methyl 4-formyl-2-(trifluoromethyl)benzoate (1.4 g, 42% yield) as colorless oil.

1H NMR (400 MHz, CHLOROFORM-d) δ ppm 10.15-10.09 (m, 1H), 8.25 (s, 1H), 8.13 (dd, J=1.1, 7.8 Hz, 1H), 7.94 (d, J=7.8 Hz, 1H), 4.02-3.96 (m, 3H)

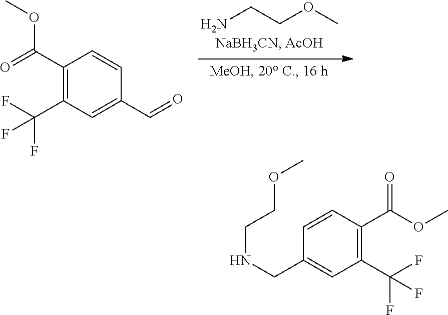

To a solution of methyl 4-formyl-2-(trifluoromethyl)benzoate (700 mg, 3.02 mmol, 1 eq) in MeOH (10 mL) were added AcOH (181.07 mg, 3.02 mmol, 172.45 uL, 1 eq), 4A MS (700 mg) and 2-methoxyethan-1-amine (226.47 mg, 3.02 mmol, 262.12 uL, 1 eq) at 0° C. The mixture was stirred at 10° C. for 3 hr, then NaBH3CN (378.96 mg, 6.03 mmol, 2 eq) was added in portions at 10° C. and the mixture was stirred at 10° C. for 12 hr. The reaction mixture was filtered concentrated. The residue was purified by prep-HPLC (column: Waters Xbridge C18 150*50 mm*10 um; mobile phase: [water(10 mM NH4HCO3)-ACN];B %: 30%-60%, 11 min) to give the intermediate compound 110 (500 mg, 57% yield) as colorless oil.

LCMS (ESI position ion) m/z: (M+H)+: 292.0 (calculated: 291.11)

1H NMR (400 MHz, CHLOROFORM-d) δ ppm 7.77 (d, J=7.9 Hz, 1H), 7.75 (s, 1H), 7.60 (d, J=7.9 Hz, 1H), 3.93 (s, 3H), 3.92 (s, 2H), 3.55-3.50 (m, 2H), 3.37 (s, 3H), 2.82-2.78 (m, 2H)

Intermediate Compound 111:

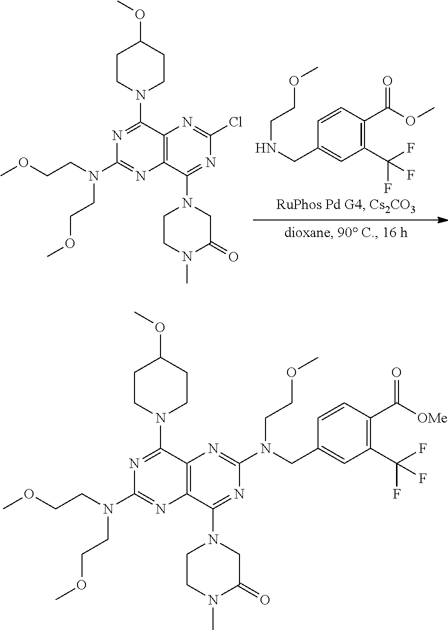

To a mixture of intermediate compound 52 (50 mg, 95.60 umol, 1 eq), intermediate compound 110 (41.77 mg, 143.40 umol, 1.5 eq) in dioxane (1.5 mL) was added Cs2CO3 (93.44 mg, 286.79 umol, 3 eq) and Ruphos Pd G4 (16.26 mg, 19.12 umol, 0.2 eq) under N2. The mixture was stirred at 90° C. for 16 hr. The reaction mixture was filtered and concentrated. The residue was purified by prep-TLC (SiO2, EA:EtOH=10:1). (Rf=0.5) to give the intermediate compound 111 (70 mg, crude) as yellow oil.

LCMS (ESI position ion) m/z: (M+H)+: 778.5 (calculated: 777.38)

Intermediate Compound 112:

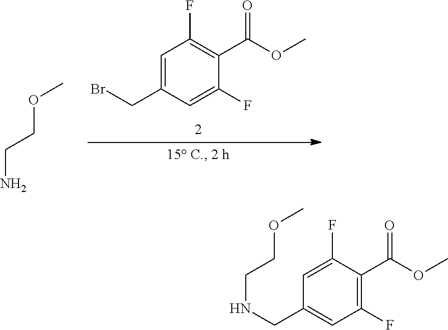

A mixture of methyl 4-(bromomethyl)-2,6-difluorobenzoate (400 mg, 1.51 mmol, eq) in 2-methoxyethanamine (2.59 g, 34.51 mmol, 3 mL, 23 eq) was stirred at 15° C. for 2 h. The mixture was concentrated in vacuum. The residue was purified by Prep-HPLC (column: Phenomenex Gemini-NX C18 75×30 mm×3 um; mobile phase: [water (0.05% ammonia hydroxide v/v)-ACN];B %: 18%-48%, 7 min) to give the intermediate compound 112 (200 mg, 50% yield) as yellow oil.

LCMS (ESI position ion) m/z: (M+H)+: 259.8 (calculated: 259.1)

1H NMR (400 MHz, CHLOROFORM-d) δ ppm 6.98 (d, J=9.4 Hz, 2H), 3.94 (s, 3H), 3.83 (s, 2H), 3.51 (t, J=4.8 Hz, 2H), 3.37 (s, 3H), 2.78 (t, J=4.8 Hz, 2H).

Intermediate Compound 113:

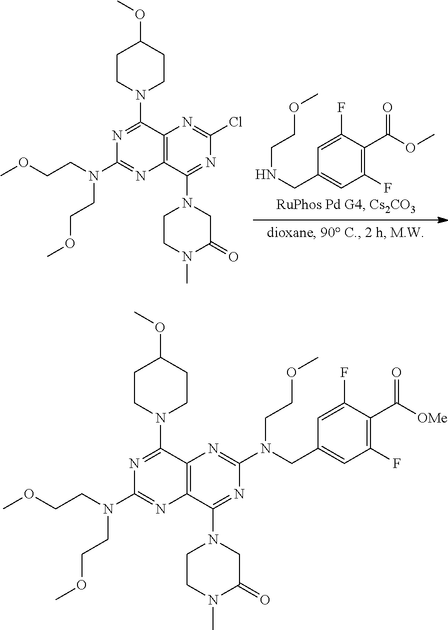 [SMILES: COCCN(CCOC)c1nc(N2CCC(OC)CC2)c2nc(Cl)nc(N3CCN(C)C(=O)C3)c2n1 ; COCCNCc1cc(F)c(C(=O)OC)c(F)c1 ; COCCN(Cc1cc(F)c(C(=O)OC)c(F)c1)c1nc(N2CCN(C)C(=O)C2)c2nc(N(CCOC)CCOC)nc(N3CCC(OC)CC3)c2n1]

Intermediate compound 52 (150 mg, 286.79 umol, 1 eq), intermediate compound 112 (111.53 mg, 430.19 umol, 1.5 eq), Ruphos Pd G4 (73.17 mg, 86.04 umol, 0.3 eq) and Cs2CO3 (280.33 mg, 860.38 umol, 3 eq) were taken up into a microwave tube in dioxane (3 mL). The sealed tube was heated at 90° C. for 2 h under microwave. The mixture was filtered and concentrated in vacuum. The residue was purified by Prep-TLC (Ethyl acetate/Ethanol=10/1) to give the intermediate compound 113 (108 mg, 50% yield) as yellow solid.

### Example I.2. Synthesis of Final Compounds

Compound 1:

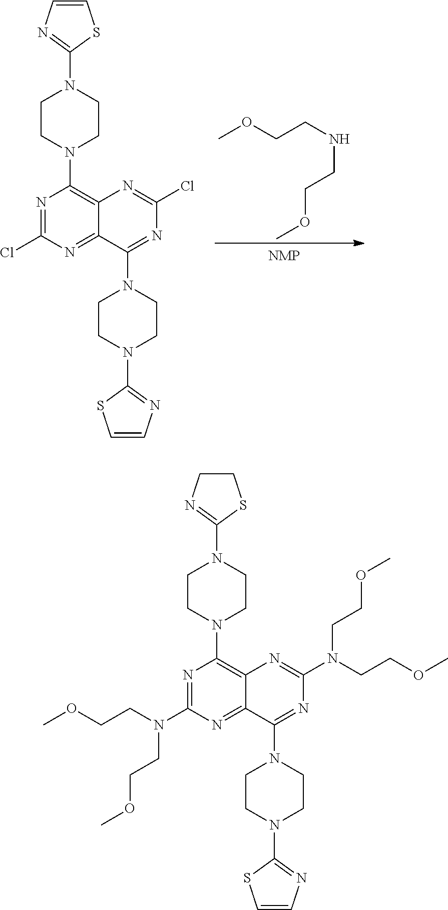

A mixture of intermediate compound 1 (180 mg, 336.15 μmol, 1 eq), bis(2-methoxyethyl)amine (405 mg, 3.04 mmol, 449.00 μl, 9.05 eq) and DIEA (174 mg, 1.35 mmol, 234.50 μl, 4.01 eq) in NMP (0.5 mL) was stirred at 180° C. for 3 h under microwave. The reaction mixture was filtered. The filtrate was purification by pre-HPLC (column: Phenomenex Synergi C18 150*25*10 um; mobile phase: [water(0.225% FA)-ACN];B %: 38%-68%, 9 min) to give the Compound 1 (9.1 mg, 3.5% yield, 94% purity) as a yellow solid. LCMS (ESI position ion) m/z: (M+H)+: 729.3 (calculated: 728.34)

Compound 2:

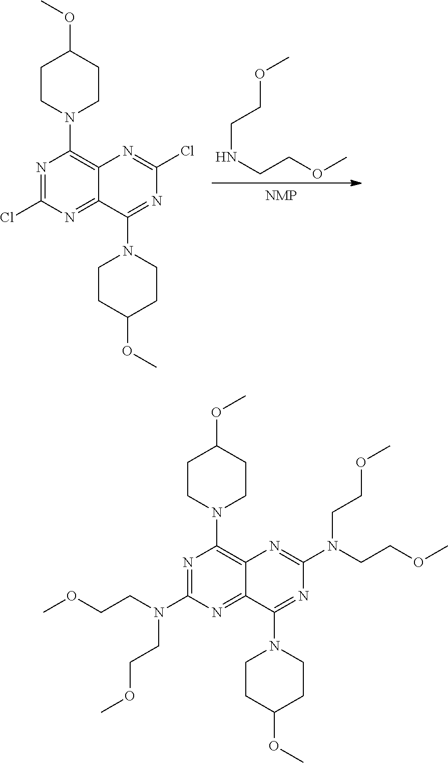

To a solution of intermediate compound 2 (200 mg, 468.03 μmol, 1 eq) and bis(2-methoxyethyl)amine (562 mg, 4.22 mmol, 623.06 μl, 9.02 eq) in NMP (0.5 mL) was sealed and heated in microwave at 180° C. for 2 hr. The reaction mixture was quenched by addition water 30 mL at 25° C., and extracted with ethyl acetate 40 mL (20 mL*2). The combined organic layers were dried over [Na2SO4], filtered and concentrated under reduced pressure to give a residue. The residue was purified by prep-TLC (SiO2, Petroleum ether/Ethyl acetate=5/1, Rf=0.11) to give a solid. The solid was further purification by pre-HPLC (column: Phenomenex Synergi C18 150*25*10 um; mobile phase: [water (0.05% HCl)-ACN]; B %: 36%-56%, 7 min). to give Compound 2 (40.9 mg, 13.7% yield, 97.4% purity) as a yellow solid.

LCMS (ESI position ion) m/z: (M+H)+: 621.4 (calculated: 620.40)

1H NMR (400 MHz, CHLOROFORM-d) δ ppm 1.75-1.63 (m, 4H) 2.05-1.93 (m, 4H) 3.37-3.28 (m, 12H) 3.39 (s, 6H) 3.46 (m, 2H) 3.63-3.52 (m, 12H) 3.76 (m, 8H) 4.68 (m, 4H).

Compound 3:

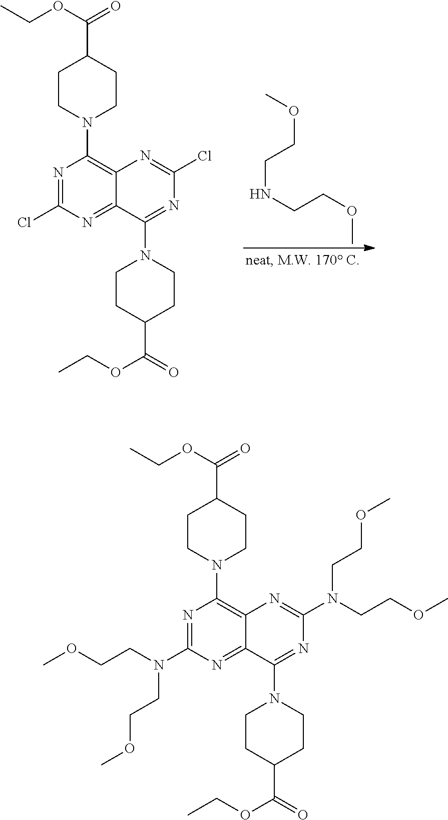

The mixture of intermediate compound 3 (230 mg, 449.75 μmol, 1 eq) and bis(2-methoxyethyl)amine (500 mg, 3.75 mmol, 8.35 eq) was heated at 100° C. for 1 hr and at 170° C. in microwaves oven for 1 h. The reaction mixture was poured into water (10 mL) and extracted with ethylacetate (20 mL, 3 times). After concentration the yellow solid obtained was purified by prep-HPLC (column: Phenomenex Synergi C18 150*25*10 um; mobile phase: [water(0.225% FA)-ACN];B %: 35%-65%, 10 min) to give the Compound 3 (45 mg, 14.2% yield, 100% purity) as a yellow solid.

LCMS (ESI position ion) m/z: (M+H)+: 705.3 (calculated: 704.42)

Compound 4:

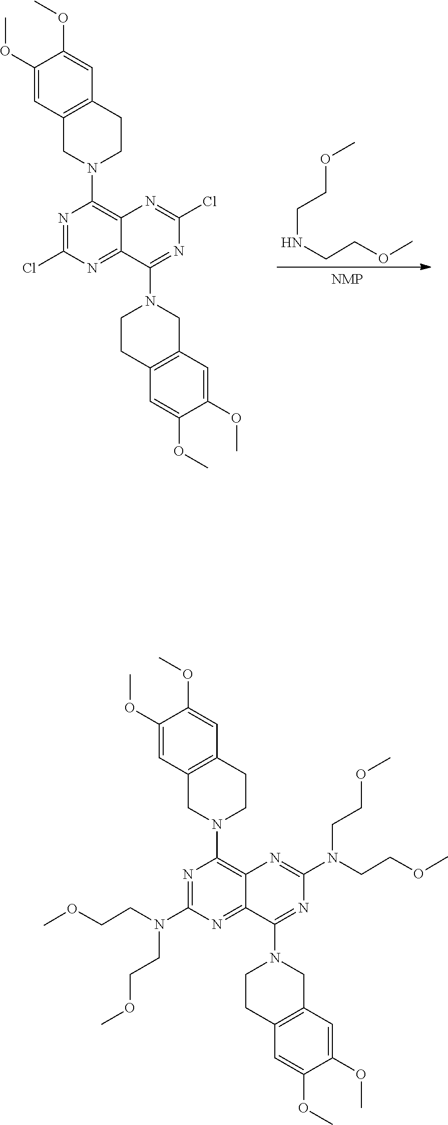

To a solution of intermediate compound 8 (200 mg, 342.78 μmol, 1 eq) and bis(2-methoxyethyl)amine (411 mg, 3.09 mmol, 455.65 μl, 9 eq) in NMP (0.5 mL) was sealed and heated in microwave at 180° C. for 4 hr. The reaction mixture was quenched by addition water 30 mL at 25° C., and extracted with ethyl acetate (20 mL, 2 times). The combined organic layers were dried over Na2SO4, filtered and concentrated under reduced pressure. The residue was purified by prep-HPLC (column: UniSil 3-100 C18 Ultra (150*25 mm*3 um); mobile phase: [water (0.225% FA)-ACN]; B %: 43%-73%, 10 min) and then by prep-HPLC (column: Waters XBridge 150*25 mm*5 um; mobile phase: [water (10 mM NH4HCO3)-ACN]; B %: 70%-100%, 10 min) to give the Compound 4 (4.7 mg, 1.7% yield, 97% purity) as a yellow solid.

LCMS (ESI position ion) m/z: (M+H)+: 777.4 (calculated: 776.42)

Compound 11:

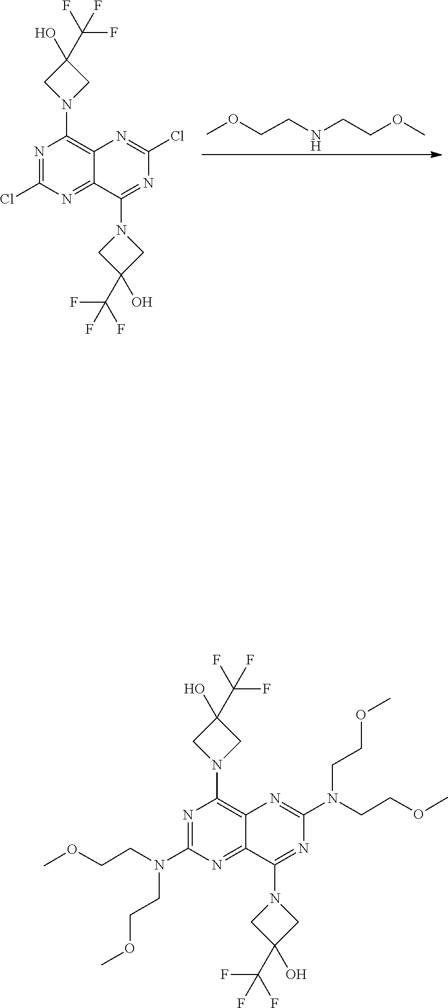

To a solution of intermediate compound 30 (100 mg, 208.70 μmol, 1 eq), bis(2-methoxyethyl)amine (250 mg, 1.88 mmol, 277.16 μl, 8.99 eq) and DIEA (67 mg, 518.40 μmol, 90.30 μl, 2.48 eq) in NMP (1 mL) was sealed and heated in microwave at 180° C. for 4 hr. The reaction mixture was diluted with water 20 mL and extracted with ethyl acetate (10 mL, 3 times). The combined organic layers were dried over Na2SO4, filtered and concentrated under reduced. The residue was purified by prep-HPLC (column: Phenomenex luna C18 150*25 mm*10 um; mobile phase: [water (0.05% HCl)-ACN]; B %: 32%-62%, 10 min) to give the Compound 11 (14.4 mg, 9.5% yield, 93% purity) as a brown gum.

LCMS (ESI position ion) m/z: (M+H)+: 673.3 (calculated: 672.28)

1H NMR (400 MHz, CHLOROFORM-d) δ ppm 3.34-3.33 (m, 12H) 3.50-3.45 (m, 2H) 3.98-3.51 (m, 19H) 5.25-4.25 (m, 9H) 8.00-7.28 (m, 1H) 10.29-9.90 (m, 1H)

Compound 12:

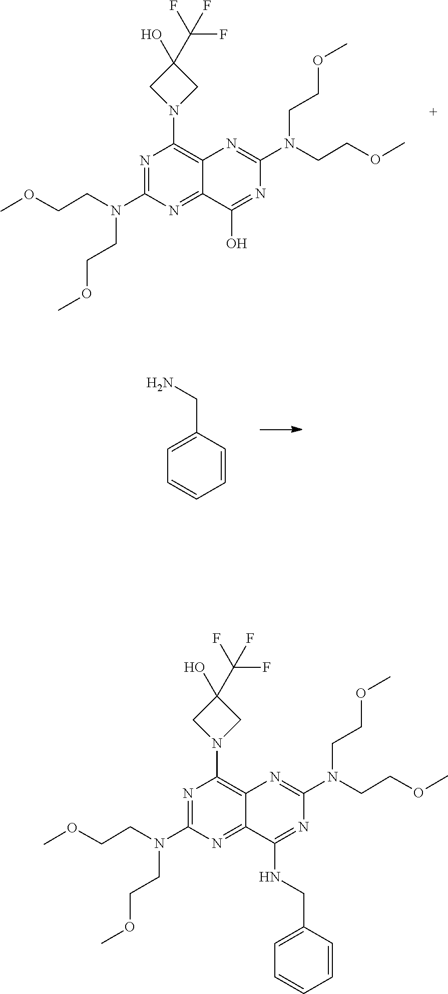

To a mixture of intermediate compound 29 (10 mg, 18.20 μmol, 1 eq), DBU (8 mg, 52.55 μmol, 7.92 μl, 2.89 eq) and BOP (11 mg, 24.87 μmol, 1.37 eq) in DMF (0.4 mL) was stirred at 16° C. for 0.5 hr. And the mixture was added phenylmethanamine (6 mg, 55.99 μmol, 6.10 μl, 3.08 eq) in DMF (0.1 mL) was stirred at 16° C. for 1.5 hr. The residue was purified by prep-HPLC (column: Waters XBridge 150*25 mm*5 um; mobile phase: [water (10 mM NH4HCO3)-ACN]; B %: 58%-88%, 9 min). The crude product was purified by prep-TLC (SiO2, Petroleum ether/Ethyl acetate=1:1) to give the Compound 12 (4.1 mg, 33.4% yield, 95% purity) as a yellow gum.

LCMS (ESI position ion) m/z: (M+H)+: 639.4 (calculated: 638.32)

Compound 13:

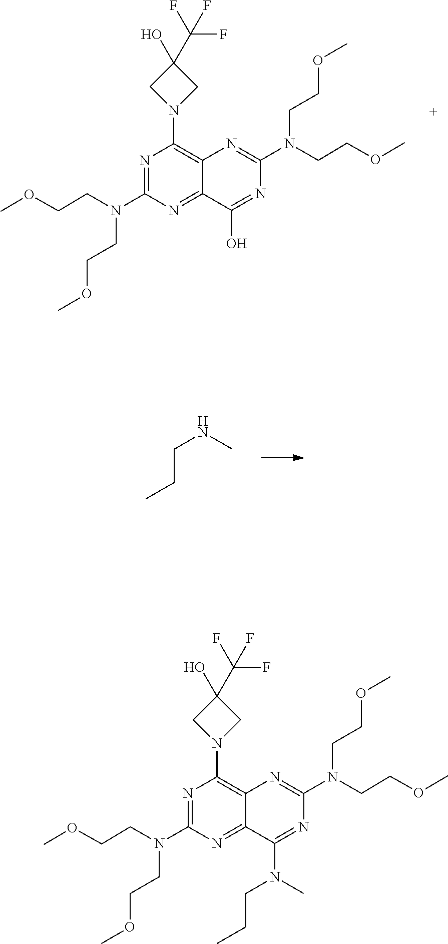

To a mixture of intermediate compound 29 (10 mg, 18.20 μmol, 1 eq), DBU (8 mg, 52.55 μmol, 7.92 μl, 2.89 eq) and BOP (11 mg, 24.87 μmol, 1.37 eq) in DMF (0.4 mL) was stirred at 16° C. for 0.5 hr. And a mixture of N-methylpropan-1-amine (4 mg, 54.69 μmol, 5.61 μl, 3.01 eq) in DMF (0.1 mL) was added before stirring at 16° C. for 1.5 hr. The reaction mixture was purified by prep-HPLC (column: Waters XBridge 150*25 mm*5 um; mobile phase: [water (10 mM NH4HCO3)-ACN]; B %: 66%-96%, 9 min) to give the Compound 13 (4.5 mg, 38.2% yield, 94.5% purity) as a yellow gum. LCMS (ESI position ion) m/z: (M+H)+: 605.5 (calculated: 604.33)

Compound 14:

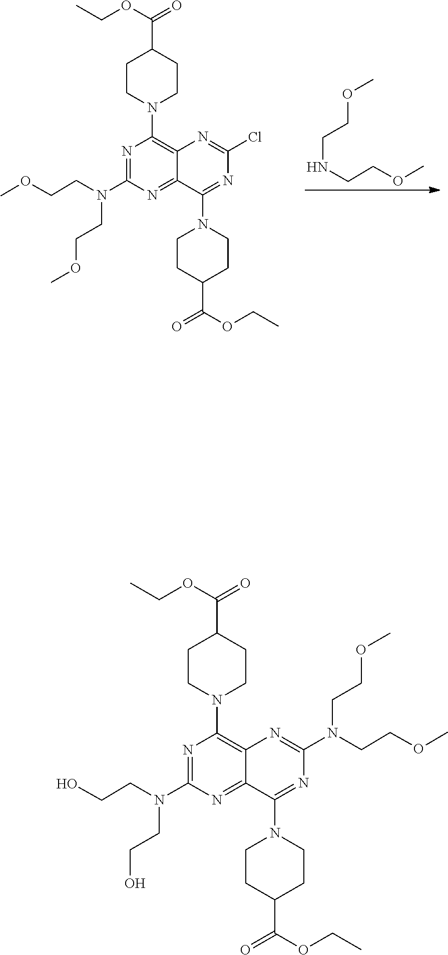

A mixture of intermediate compound 7 (50 mg, 86.20 μmol, 1 eq) in bis(2-methoxyethyl)amine (84 mg, 630.69 μmol, 93.13 μl, 7.32 eq) was stirred at 170° C. for 1 h under microwave. The reaction mixture was purification by pre-HPLC (column: 3_Phenomenex Luna C18 75*30 mm*3 um; mobile phase: [water(0.05% HCl)-ACN];B %: 31%-51%, 6.5 min) to give the Compound 14 (11.6 mg, 19% yield, 95.5% purity) as a yellow oil.

LCMS (ESI position ion) m/z: (M+H)+: 677.2 (calculated: 676.39)

Compound 15:

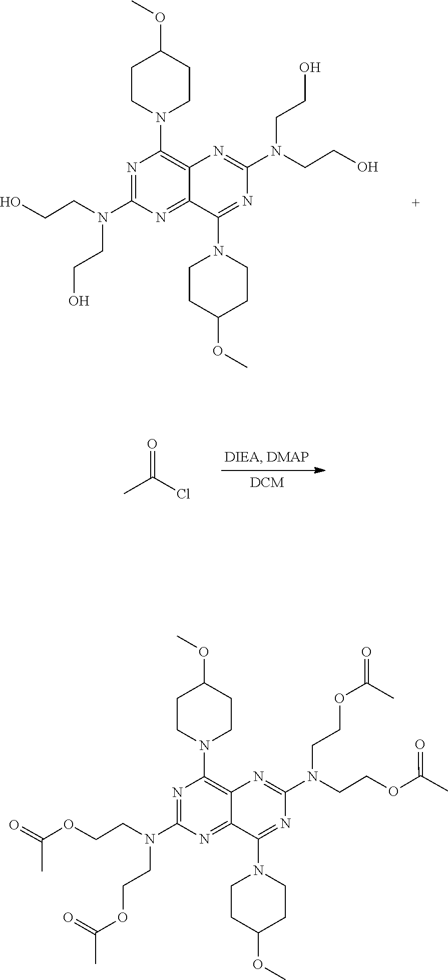

To a mixture of intermediate compound 4 (70 mg, 123.96 μmol, 1 eq), DIEA (352.48 mg, 2.73 mmol, 475.03 μl, 22 eq) and DMAP (2.80 mg, 22.92 μmol, 1.85e-1 eq) in DCM (1 mL) was added acetyl chloride (110.00 mg, 1.40 mmol, 100 μl, 11.30 eq) dropwise at −5° C. under N2. The mixture was stirred at −5° C. for 1 hr, then heated to 25° C. and stirred for 2.5 hr. The reaction was quenched by poured into water (20 mL), and then extracted with DCM (20 mL, 2 times). The combined organic phase was washed with brine (10 mL), dried with anhydrous Na2SO4, filtered and concentrated in vacuum. The residue was purified by prep. HPLC (column: Waters XBridge 150*25 mm*5 um; mobile phase: [water(10 mM NH4HCO3)-ACN];B %: 50%-80%, 10 min) to give the Compound 15 (4.1 mg, 4.3% yield, 96% purity) as a yellow solid.

LCMS (ESI position ion) m/z: (M+H)+: 733.4 (calculated: 732.38)

Compound 19:

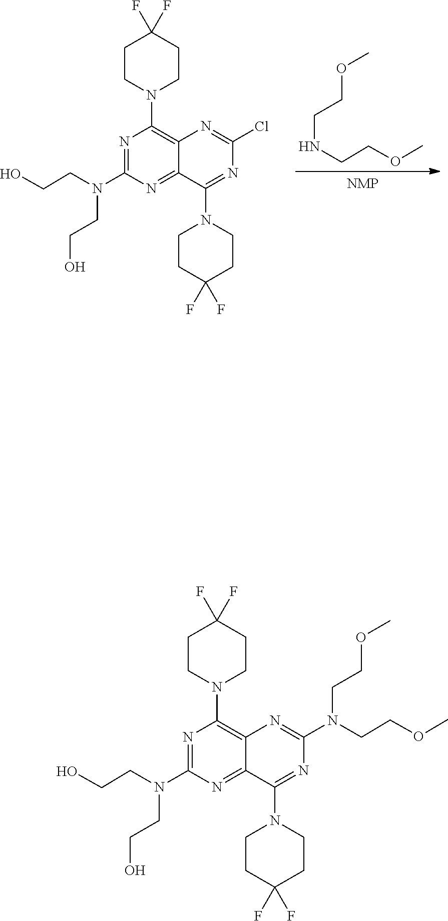

To a solution of intermediate compound 6 (50 mg, 98.44 μmol, 1 eq), bis(2-methoxyethyl)amine (120 mg, 900.98 μmol, 133.04 μl, 9.15 eq) in NMP (0.2 mL) was sealed and heated in microwave at 180° C. for 2 hr. The reaction mixture was filtered and the residue was purified by prep-HPLC (column: Waters XBridge 150*25 mm*5 um; mobile phase: [water (10 mM NH4HCO3)-ACN]; B %: 42%-72%, 10 min) to give the Compound 19 (16.4 mg, 27.5% yield, 100% purity) as a yellow solid.

LCMS (ESI position ion) m/z: (M+H)+: 605.3 (calculated: 604.31)

Compound 27:

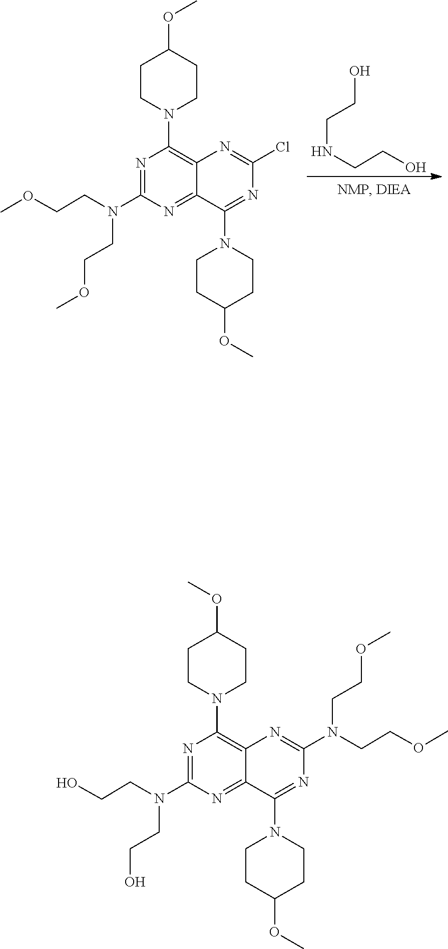 [SMILES: COCCN(CCOC)c1nc(N2CCC(OC)CC2)c2nc(Cl)nc(N3CCC(OC)CC3)c2n1 ; OCCNCCO ; COCCN(CCOC)c1nc(N2CCC(OC)CC2)c2nc(N(CCO)CCO)nc(N3CCC(OC)CC3)c2n1]

To a solution of intermediate compound 9 (100 mg, 190.82 μmol, 1 eq) and 2,2′-azanediylbis(ethan-1-ol) (181 mg, 1.72 mmol, 166.06 μl, 9.02 eq) in NMP (0.5 mL) and DIEA (62 mg, 479.73 μmol, 83.56 μl, 2.51 eq) was sealed and heated in microwave at 180° C. for 2 hr and at 200° C. for 2 hr. The residue was purified by prep-HPLC (column: Waters XBridge 150*25 mm*5 um; mobile phase: [water (10 mM NH4HCO3)-ACN]; B %: 33%-63%, 9 min) to give the Compound 27 (48.2 mg, 42.6% yield, 100% purity) as a yellow oil. LCMS (ESI position ion) m/z: (M+H)+: 593.2 (calculated: 592.37)

Compound 34:

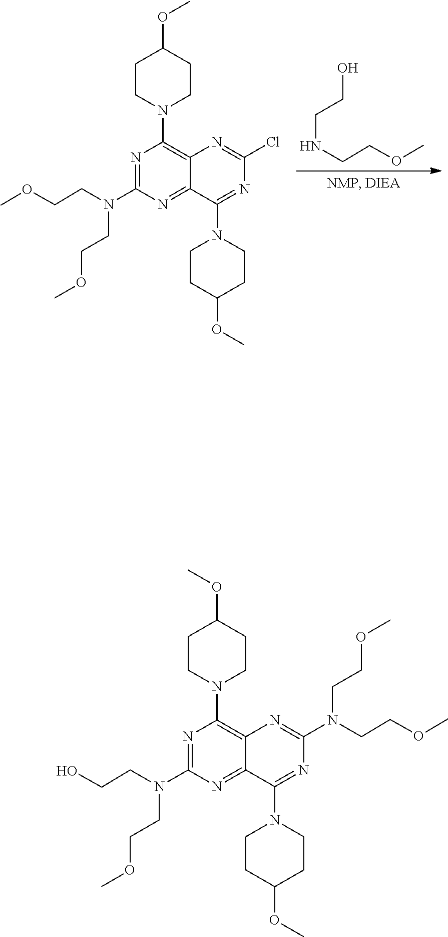

To a solution of intermediate compound 9 (100 mg, 190.82 μmol, 1 eq) and 2-((2-methoxyethyl)amino)ethan-1-ol (90 mg, 755.27 μmol, 3.96 eq) in NMP (0.5 mL) and DIEA (62 mg, 479.72 μmol, 83.56 μl, 2.51 eq) was sealed and heated in microwave at 180° C. for 2 hr and at 200° C. for 4.5 hr. The reaction mixture was diluted with water 50 mL and extracted with ethyl acetate (20 mL 3 times). The combined organic layers were dried over Na2SO4, filtered and concentrated under reduced pressure. The residue was purified by prep-HPLC (column: 3_Phenomenex Luna C18 75*30 mm*3 um; mobile phase: [water (0.05% HCl)-ACN]; B %: 29%-49%, 6.5 min) to give the Compound 34 (12.3 mg, 10% yield, 95% purity) as a yellow oil.

LCMS (ESI position ion) m/z: (M+H)+: 607.5 (calculated: 606.39)

Compound 37:

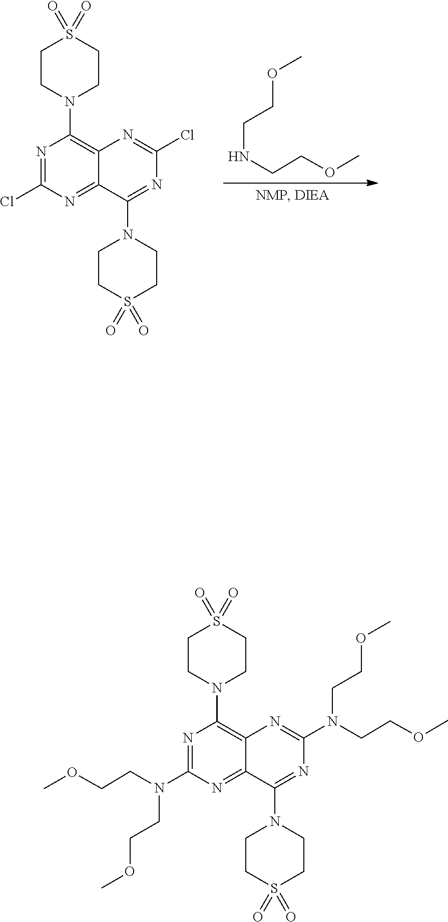

A solution of intermediate compound 10 (69 mg, 147.64 μmol, 1 eq) and bis(2-methoxyethyl)amine (176 mg, 1.32 mmol, 195.12 μl, 8.95 eq) in NMP (1 mL) and DIEA (48 mg, 371.40 μmol, 64.69 μl, 2.52 eq) was sealed and heated in microwave at 180° C. for 2 hr and at 200° C. for 2 hr. The reaction mixture was diluted with water (50 mL) and extracted with ethyl acetate (20 mL, 3 times). The combined organic layers were dried over Na2SO4, filtered and concentrated under reduced pressure. The residue was purified by prep-HPLC (column: 3_Phenomenex Luna C18 75*30 mm*3 um; mobile phase: [water (0.05% HCl)-ACN]; B %: 27%-47%, 6.5 min) to give the Compound 37 (46 mg, 47.1% yield, 100% purity) as a yellow solid.

LCMS (ESI position ion) m/z: (M+H)+: 661.3 (calculated: 660.27)

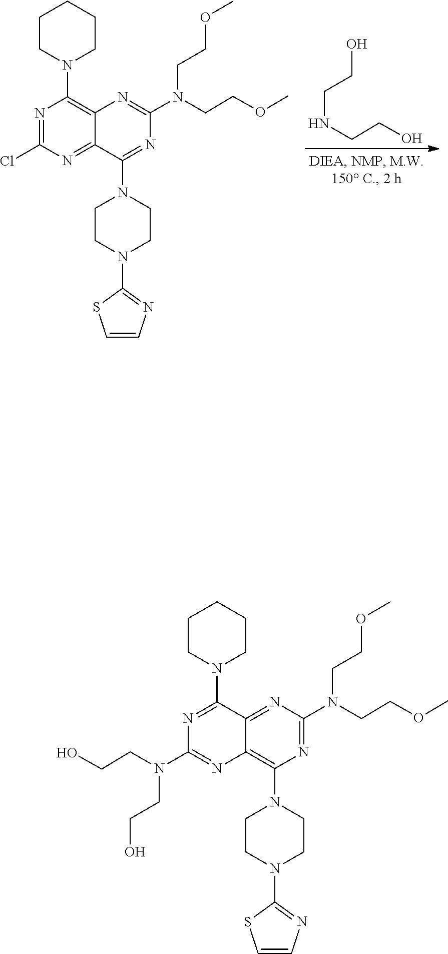

Compound 38:

A mixture of intermediate compound 13 (100 mg, 176.97 μmol, 1 eq) and 2,2′-azanediylbis(ethan-1-ol) (73.87 mg, 702.59 μmol, 67.77 μl, 3.97 eq), DIEA (70.87 mg, 548.37 μmol, 95.51 μl, 3.10 eq) in NMP (0.5 mL) was stirred at 150° C. for 2 hr under microwave. The mixture was purified by pre-HPLC (column: Phenomenex luna C18 150*25 mm*10 um; mobile phase: [water (0.1% TFA)-ACN];B %: 18%-48%, 10 min) to give the Compound 38 (40.4 mg, 18% yield, 94% purity) as a yellow gum.

LCMS (ESI position ion) m/z: (M+H)+: 617.4 (calculated: 616.33)

Compound 39:

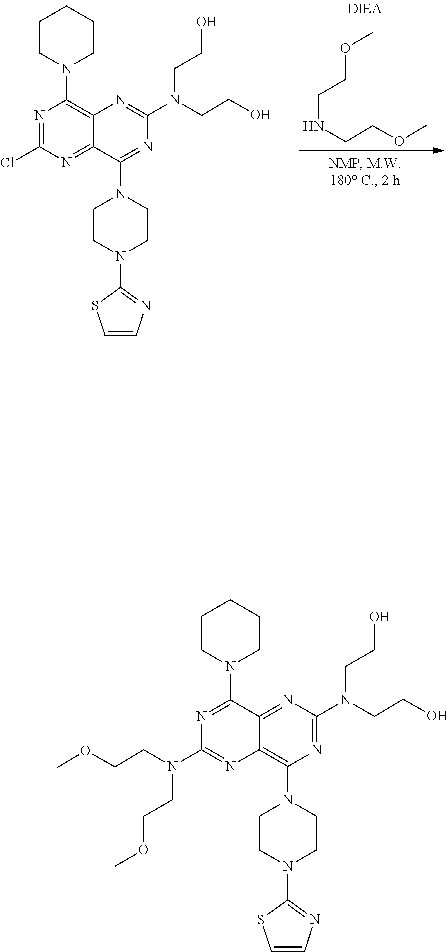

A mixture of intermediate compound 15 (0.24 g, 461.49 μmol, 1 eq) and bis(2-methoxyethyl)amine (245 mg, 1.84 mmol, 271.62 μl, 3.99 eq), DIEA (184 mg, 1.42 mmol, 247.98 μl, 3.08 eq) in NMP (1 mL) was stirred at 180° C. for 2 hr under microwave. The mixture was purified by pre-HPLC (column: Waters XBridge C18 150*50 mm*10 um; mobile phase: [water (10 mM NH4HCO3)-ACN]; B %: 45%-75%, 11.5 min, Rt=0.807 min was collected) to give a solid with 92.2% purity which was purified further by a second pre-HPLC (column: Phenomenex luna C18 150*25 mm*10 um; mobile phase: [water (0.1% TFA)-ACN]; B %: 18%-48%, 10 min) to give the Compound 39 (142.7 mg, 40% yield, 95% purity) as a yellow solid.

LCMS (ESI position ion) m/z: (M+H)+: 617.2 (calculated: 616.33). ¹H NMR (400 MHz, CHLOROFORM-d) δ ppm 1.705 (s, 6H) 3.369 (s, 6H) 3.57-3.60 (m, 4H) 3.637-3.663 (m, 4H) 3.781 (m, 7H) 3.895-3.906 (m, 8H) 3.917 (brs, 2H) 4.327 (brs, 3H) 6.609-6.618 (d, 1H J=3.6) 7.229-7.238 (d, 1H J=3.6)

General Procedure I

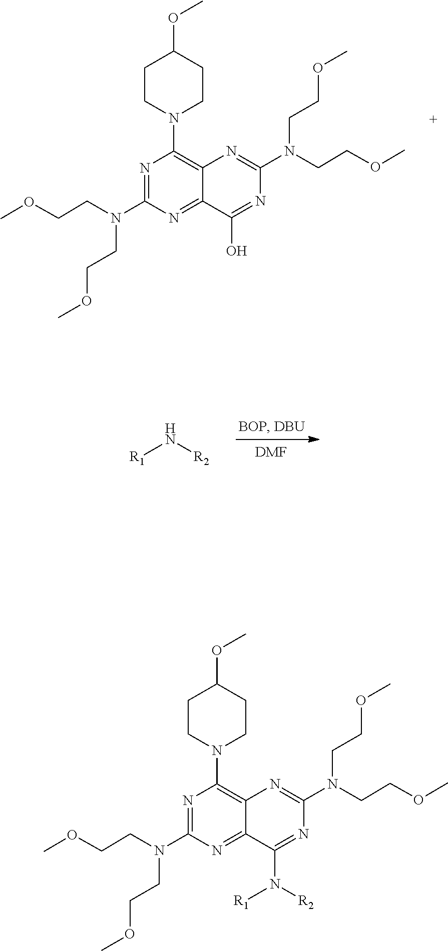

To a solution of intermediate compound 16 (30 mg, 95.49 μmol, 1 eq), BOP (1.3 eq) and DBU (1.5 eq) in DMF (0.4 mL) was stirred at 20° C. for 1 hr before addition of the required amine (3.00 eq) in DMF (0.1 mL). The reaction mixture was stirred at 20° C. for 14 hr. The reaction mixture was diluted with water 5 mL and extracted with ethyl acetate (2 mL, 3 times). The combined organic layers were dried over Na2SO4, filtered and concentrated under reduced pressure. The residue was purified by preparative HPLC following the purification method A, B or C to give the desired Compound as indicated in the Table 2. Compounds that were obtained according to General Procedure I is presented in Table 2.

TABLE 2

| | LCMS (ESI | | | | | |
|---|---|---|---|---|---|---|
| | position ion) | Calculated | | Method of | | |
| Compound | m/z: (M + H)+ | Molar Mass | Amines | purification | Appearance | Purity |
| Compound | 621.3 | 620.785 | (tetrahydro-2H-pyran-4- | B | yellow oil | 99.29% |
| 46 | | | yl)methanamine | | | |
| Compound | 643.2 | 642.791 | (4- | A | yellow oil | 98.92% |
| 47 | | | methoxyphenyl)methanamine | | | |
| Compound | 643.4 | 642.791 | (3- | A | yellow oil | 99.51% |
| 48 | | | methoxyphenyl)methanamine | | | |
| Compound | 613.3 | 612.765 | phenylmethanamine | B | yellow oil | 98.07% |
| 49 | | | | | | |
| Compound | 593.7 | 592.775 | 2,2-dimethylpropan-1- | B | yellow oil | 100.00% |
| 50 | | | amine | | | |
| Compound | 595.2 | 594.748 | 3-methoxypropan-1-amine | A | yellow oil | 100.00% |
| 51 | | | | | | |
| Compound | 609.6 | 608.774 | 3-ethoxypropan-1-amine | A | yellow oil | 100.00% |
| 52 | | | | | | |
| Compound | 682.1 | 681.751 | (6- | A | yellow | 100.00% |
| 53 | | | (trifluoromethyl)pyridin- | | solid | |
| | | | 3-yl)methanamine | | | |
| Compound | 579.2 | 578.748 | butan-1-amine | A | yellow oil | 100.00% |
| 54 | | | | | | |
| Compound | 605.5 | 604.786 | cyclopentylmethanamine | A | yellow | 100.00% |
| 55 | | | | | solid | |
| Compound | 643.4 | 642.791 | 2-phenoxyethan-1-amine | A | yellow | 100.00% |
| 56 | | | | | solid | |
| Compound | 607.4 | 606.758 | (tetrahydrofuran- | A | yellow | 100.00% |
| 57 | | | 3-yl)methanamine | | solid | |
| Compound | 620.3 | 619.781 | thiazol-2-ylmethanamine | A | yellow oil | 100.00% |
| 58 | | | | | | |
| Compound | 620.3 | 619.781 | thiazol-5-ylmethanamine | A | yellow | 99.00% |
| 59 | | | | | solid | |
| Compound | 604.4 | 603.715 | oxazol-5-ylmethanamine | B | yellow gum | 100.00% |
| 60 | | | | | | |
| Compound | 614.4 | 613.753 | pyridin-4-ylmethanamine | B | yellow gum | 100.00% |
| 61 | | | | | | |
| Compound | 614.4 | 613.753 | pyridin-3-ylmethanamine | B | yellow gum | 100.00% |
| 62 | | | | | | |
| Compound | 628.5 | 627.779 | 2-(pyridin-3-yl)ethan-1- | B | yellow | 100.00% |
| 63 | | | amine | | solid | |
| Compound | 628.5 | 627.779 | 2-(pyridin-4-yl)ethan-1- | B | yellow gum | 100.00% |
| 64 | | | amine | | | |
| Compound | 617.6 | 616.757 | (1-methyl-1H-pyrazol-4- | B | yellow gum | 100.00% |
| 65 | | | yl)methanamine | | | |
| Compound | 620.5 | 619.781 | thiazol-4-ylmethanamine | B | yellow | 100.00% |
| 66 | | | | | solid | |
| Compound | 551.3 | 550.695 | dimethylamine | A | yellow | 100.00% |
| 67 | | | | | solid | |
| Compound | 579.3 | 578.748 | N-methylpropan-1-amine | A | yellow | 100.00% |
| 68 | | | | | solid | |
| Compound | 593.3 | 592.732 | morpholine | B | yellow oil | 96.79% |
| 69 | | | | | | |
| Compound | 606.3 | 605.731 | piperazin-2-one | A | yellow | 100.00% |
| 70 | | | | | solid | |
| Compound | 607.2 | 606.758 | piperidin-4-ol | A | yellow | 100.00% |
| 71 | | | | | solid | |
| Compound | 620.2 | 619.757 | 1-methylpiperazin-2-one | A | yellow | 100.00% |
| 72 | | | | | solid | |
| Compound | 628.3 | 627.779 | N-methyl-1-(pyridin-2- | A | yellow oil | 92.00% |
| 73 | | | yl)methanamine | | | |
| Compound | 634.2 | 633.784 | 1-(piperazin-1-yl)ethan- | A | yellow | 100.00% |
| 74 | | | 1-one | | solid | |
| Compound | 634.2 | 633.784 | piperidine-4-carboxamide | B | yellow oil | 100.00% |
| 75 | | | | | | |
| Compound | 635.5 | 634.812 | 4-(methoxymethyl) | C | yellow | 100.00% |
| 76 | | | piperidine | | solid | |
| Compound | 641.1 | 640.797 | thiomorpholine 1,1-dioxide | A | yellow | 100.00% |
| 77 | | | | | solid | |
| Compound | 669.2 | 668.851 | 4- | C | yellow | 93.00% |
| 78 | | | (methylsulfonyl)piperidine | | solid | |
| Compound | 607.2 | 606.758 | 1,4-oxazepane | B | yellow oil | 100.00% |
| 79 | | | | | | |
| Compound | 607.1 | 606.758 | 3-methoxypyrrolidine | B | yellow | 97.00% |
| 80 | | | | | solid | |
| Compound | 628.3 | 627.779 | N-methyl-1-(pyridin-3- | B | yellow oil | 95.00% |
| 81 | | | yl)methanamine | | | |
| Compound | 628.2 | 627.779 | N-methyl-1-(pyridin-4- | A | yellow | 100.00% |
| 82 | | | yl)methanamine | | solid | |
| Compound | 642.2 | 641.806 | N-methyl-2-(pyridin-2- | B | yellow oil | 97.00% |
| 83 | | | yl)ethan-1-amine | | | |
| Compound | 615.2 | 614.741 | pyrazin-2-ylmethanamine | B | yellow | 100.00% |
| 84 | | | | | solid | |
| Compound | 579.4 | 578.748 | diethylamine | A | yellow | 100.00% |
| 85 | | | | | solid | |
| Compound | 593.2 | 592.732 | 3-methoxyazetidine | A | yellow | 100.00% |
| 86 | | | | | solid | |
| Compound | 607.2 | 606.758 | 2-methylmorpholine | A | yellow | 100.00% |
| 87 | | | | | solid | |
| Compound | 648.4 | 647.81 | N-(piperidin-4- | A | yellow | 100.00% |
| 88 | | | yl)acetamide | | solid | |
| Compound | 577.2 | 576.732 | pyrrolidine | C | yellow | 100.00% |
| 89 | | | | | solid | |
| Compound | 616.3 | 615.769 | piperidine-4-carbonitrile | C | yellow | 93.00% |
| 90 | | | | | solid | |
| Compound | 591.2 | 590.759 | 3-methylpyrrolidine | A | yellow | 100.00% |
| 91 | | | | | solid | |
| Compound | 593.4 | 592.732 | azetidin-3-ylmethanol | A | yellow | 100.00% |
| 92 | | | | | solid | |
| Compound | 593.2 | 592.732 | (R)-pyrrolidin-3-ol | A | yellow | 100.00% |
| 93 | | | | | solid | |
| Compound | 593.2 | 592.732 | pyrrolidin-3-ol | B | yellow oil | 100.00% |
| 94 | | | | | | |
| Compound | 617.3 | 616.757 | 2-(1H-imidazol-5-yl)ethan- | C | yellow oil | 98.00% |
| 95 | | | 1-amine | | | |
| Compound | 621.4 | 620.785 | 3-methoxypiperidine | B | yellow | 100.00% |
| 96 | | | | | solid | |
| Compound | 633.2 | 632.796 | 2-oxa-6- | C | yellow oil | 100.00% |
| 97 | | | azaspiro[3.5]nonane | | | |
| Compound | 648.4 | 647.81 | 1-(1,4-diazepan-1- | C | yellow | 100.00% |
| 98 | | | yl)ethan-1-one | | solid | |
| Compound | 581.4 | 580.696 | 3-fluoroazetidine | B | yellow oil | 100.00% |
| 99 | | | | | | |
| Compound | 591.2 | 590.759 | 3,3-dimethylazetidine | B | yellow oil | 98.00% |
| 100 | | | | | | |
| Compound | 593.5 | 592.732 | 3-methylazetidin-3-ol | C | yellow oil | 100.00% |
| 101 | | | | | | |
| Compound | 595.5 | 594.723 | (S)-3-fluoropyrrolidine | C | yellow | 100.00% |
| 102 | | | | | solid | |
| Compound | 595.5 | 594.723 | (R)-3-fluoropyrrolidine | A | yellow | 100.00% |
| 103 | | | | | solid | |
| Compound | 595.2 | 594.723 | 3-fluoropyrrolidine | A | yellow | 100.00% |
| 104 | | | | | solid | |
| Compound | 599.4 | 598.687 | 3,3-difluoroazetidine | B | yellow oil | 100.00% |
| 105 | | | | | | |
| Compound | 607.2 | 606.758 | (S)-3-methoxypyrrolidine | A | yellow | 100.00% |
| 106 | | | | | solid | |
| Compound | 607.2 | 606.758 | (R)-3-methoxypyrrolidine | A | yellow | 100.00% |
| 107 | | | | | solid | |
| Compound | 609.3 | 608.749 | 4-fluoropiperidine | C | yellow | 93.00% |
| 108 | | | | | solid | |
| Compound | 613.2 | 612.713 | 3,3-difluoropyrrolidine | A | yellow | 100.00% |
| 109 | | | | | solid | |
| Compound | 621.2 | 620.785 | cis-2,6- | C | yellow | 100.00% |
| 110 | | | dimethylmorpholine | | solid | |
| Compound | 647.1 | 646.703 | 3- | A | yellow oil | 100.00% |
| 111 | | | (trifluoromethyl)azetidin- | | | |
| | | | 3-ol | | | |
| Compound | 581.5 | 580.721 | 2-(methylamino)ethan-1-ol | B | yellow oil | 100.00% |
| 112 | | | | | | |
| Compound | 607.2 | 606.758 | 2-methylmorpholine | A | yellow | 91.00% |
| 113 | | | | | solid | |
| Compound | 629.2 | 628.767 | 5,6,7,8-tetrahydroimidazo | A | yellow | 100.00% |
| 114 | | | [1,5-a]pyrazine | | solid | |
| Compound | 621.2 | 620.785 | piperidin-3-ylmethanol | A | yellow oil | 100.00% |
| 115 | | | | | | |
| Compound | 630.2 | 629.755 | 5,6,7,8-tetrahydro- | C | yellow | 100.00% |
| 116 | | | [1,2,4]triazolo[1,5- | | solid | |
| | | | a]pyrazine | | | |
| Compound | 675.5 | 674.793 | 1,3,8- | A | yellow | 98.00% |
| 117 | | | triazaspiro[4.5]decane- | | solid | |
| | | | 2,4-dione | | | |
| Compound | 669.3 | 668.831 | 1-(pyridin-3-yl)piperazine | B | yellow gum | 100.00% |
| 118 | | | | | | |
| Compound | 670.4 | 669.819 | 2-(piperazin-1- | B | yellow | 100.00% |
| 119 | | | yl)pyrimidine | | solid | |
| Compound | 670.6 | 669.819 | 2-(piperazin-1-yl)pyrazine | B | yellow gum | 100.00% |
| 120 | | | | | | |
| Compound | 684.1 | 683.846 | 2-(piperazin-1-yl)pyridin- | B | yellow gum | 100.00% |
| 121 | | | 3-amine | | | |
| Compound | 684.4 | 683.846 | 4-methyl-2-(piperazin-1- | B | yellow gum | 100.00% |
| 122 | | | yl)pyrimidine | | | |
| Compound | 690.1 | 689.875 | 3-methyl-5-(piperazin-1- | B | yellow | 100.00% |
| 123 | | | yl)-1,2,4-thiadiazole | | solid | |
| Compound | 694.1 | 693.841 | 6-(piperazin-1- | B | yellow gum | 100.00% |
| 124 | | | yl)nicotinonitrile | | | |
| Compound | 700.2 | 699.845 | 4-methoxy-2-(piperazin-1- | B | yellow gum | 98.00% |
| 125 | | | yl)pyrimidine | | | |
| Compound | 551.2 | 550.695 | ethanamine | A | Yellow oil | 99.00% |
| 126 | | | | | | |
| Compound | 579.2 | 578.748 | 2-methylpropan-1-amine | A | yellow | 100.00% |
| 127 | | | | | solid | |
| Compound | 581.2 | 580.721 | 2-methoxyethan-1-amine | A | yellow | 100.00% |
| 128 | | | | | solid | |
| Compound | 674.4 | 673.808 | 2-methyl-5-(piperazin-1- | A | yellow | 100.00% |
| 135 | | | yl)-1,3,4-oxadiazole | | solid | |
| Compound | 607.2 | 606.758 | pyrrolidin-3-ylmethanol | A | yellow | 100.00% |
| 136 | | | | | solid | |

General Procedure II

In case of reaction with amine:

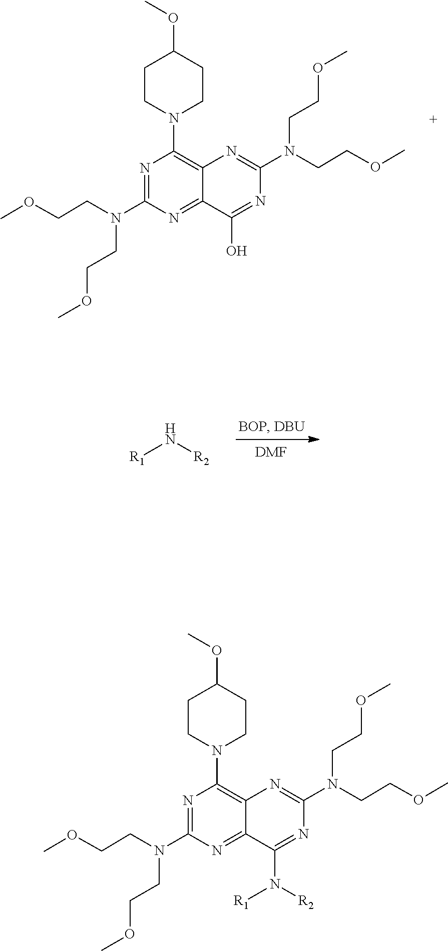

In case of reaction with alcohol:

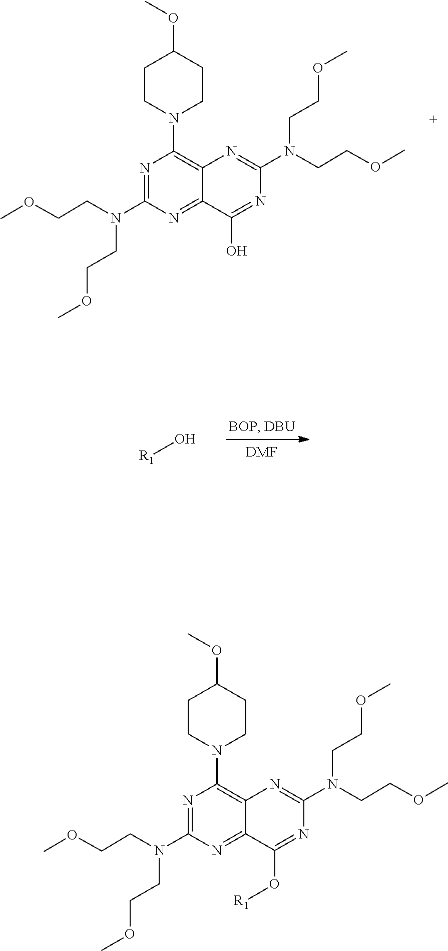

To a solution of intermediate compound 16, BOP (1.3 eq) and DBU (3 eq) in DMF (0.4 mL) was stirred at 10° C. for 30 min. After addition, amine or alcohol listed in the Table 3 in DMF (0.1 mL) was added the mixture at 10° C. The resulting mixture was stirred at 10° C. for 1.5 hr. The residue was purified by preparative HPLC following the purification method A, B or C to give the desired Compound as indicated in the Table 3. Compounds that were obtained according to General Procedure II is presented in Table 3.

TABLE 3

| | LCMS (ESI | | | | | |
|---|---|---|---|---|---|---|
| | position ion) | Calculated | | Method of | | |
| Compound | m/z: (M + H)+ | Molar Mass | Amines | purification | Appearance | Purity |
| Compound | 681.2 | 681.66 | (3,4- | A | yellow gum | 100.00% |
| 137 | | | dichlorophenyl)methanamine | | | |
| Compound | 665.4 | 665.20 | (3-chloro-4- | B | yellow | 100.00% |
| 138 | | | fluorophenyl)methanamine | | solid | |
| Compound | 649.4 | 648.75 | (3,4- | B | yellow | 100.00% |
| 139 | | | difluorophenyl)methanamine | | solid | |
| Compound | 638.3 | 637.77 | 4-(aminomethyl)benzonitrile | B | yellow gum | 100.00% |
| 140 | | | | | | |
| Compound | 647.4 | 647.21 | (3-chlorophenyl)methanamine | B | yellow | 99.20% |
| 141 | | | | | solid | |
| Compound | 631.4 | 630.76 | (3-fluorophenyl)methanamine | A | yellow oil | 100.00% |
| 142 | | | | | | |
| Compound | 697.4 | 696.76 | (3-(trifluoromethoxy)phenyl)methanamine | A | yellow oil | 98.70% |
| 143 | | | | | | |
| Compound | 679.5 | 678.77 | (3-(difluoromethoxy)phenyl)methanamine | B | yellow gum | 100.00% |
| 144 | | | | | | |
| Compound | 638.4 | 637.77 | 3-(aminomethyl)benzonitrile | A | yellow oil | 94.30% |
| 145 | | | | | | |
| Compound | 679.6 | 678.83 | (3-(1H-pyrazol-1- | A | yellow oil | 98.70% |
| 146 | | | yl)phenyl)methanamine | | | |
| Compound | 629.5 | 628.77 | 4-(aminomethyl)pyridin-2- | A | yellow | 100.00% |
| 147 | | | amine | | solid | |
| Compound | 691.4 | 690.86 | (3-(methylsulfonyl)phenyl)methanamine | C | yellow | 100.00% |
| 148 | | | | | solid | |
| Compound | 656.2 | 655.77 | 5-(aminomethyl)-2- | A | yellow oil | 100.00% |
| 149 | | | fluorobenzonitrile | | | |
| Compound | 672.2 | 672.22 | 5-(aminomethyl)-2- | A | yellow gum | 98.00% |
| 150 | | | chlorobenzonitrile | | | |
| Compound | 680.4 | 679.81 | (3-(oxazol-2- | A | yellow oil | 100.00% |
| 151 | | | yl)phenyl)methanamine | | | |
| Compound | 674.2 | 673.80 | (3,5- | A | yellow oil | 99.60% |
| 152 | | | dimethoxyphenyl)methanol | | | |
| Compound | 644.3 | 643.78 | (3- | B | brown gum | 98.26% |
| 153 | | | methoxyphenyl)methanol | | | |
| Compound | 682.1 | 682.64 | (3,4- | A | yellow | 97.00% |
| 154 | | | dichlorophenyl)methanol | | solid | |
| Compound | 648.2 | 648.19 | (3-chlorophenyl)methanol | B | yellow oil | 98.20% |
| 155 | | | | | | |
| Compound | 639.4 | 638.76 | 3-(hydroxymethyl)benzonitrile | A | yellow | 89.00% |
| 156 | | | | | solid | |
| Compound | 662.4 | 661.77 | (4-fluoro-3- | B | yellow gum | 100.00% |
| 157 | | | methoxyphenyl)methanol | | | |
| Compound | 640.4 | 639.75 | 5- | C | yellow | 100.00% |
| 158 | | | (hydroxymethyl)nicotinonitrile | | solid | |
| Compound | 696.3 | 695.81 | (3-(5-methyl-1,2,4- | A | yellow | 100.00% |
| 159 | | | oxadiazol-3- | | solid | |
| | | | yl)phenyl)methanol | | | |
| Compound | 654.1 | 654.23 | (2-chlorothiazol-5- | B | yellow gum | 99.10% |
| 160 | | | yl)methanamine | | | |
| Compound | 617.4 | 616.75 | (5-methylfuran-2- | A | yellow | 97.20% |
| 161 | | | yl)methanamine | | solid | |
| Compound | 618.4 | 617.74 | (3-methylisoxazol-5- | C | yellow oil | 100.00% |
| 162 | | | yl)methanamine | | | |
| Compound | 645.3 | 644.81 | (1-isopropyl-1H-pyrazol-4- | B | yellow oil | 100.00% |
| 163 | | | yl)methanamine | | | |
| Compound | 646.4 | 645.80 | (1-isopropyl-1H-pyrazol-4- | C | yellow oil | 97.00% |
| 164 | | | yl)methanol | | | |
| Compound | 607.4 | 606.80 | dipropylamine | A | yellow oil | 100.00% |
| 165 | | | | | | |
| Compound | 605.5 | 604.79 | N-methylcyclopentanamine | A | yellow oil | 94.00% |
| 166 | | | | | | |
| Compound | 591.5 | 590.76 | cyclopentanamine | A | yellow oil | 96.00% |
| 167 | | | | | | |
| Compound | 593.4 | 592.78 | N,2-dimethylpropan-1-amine | A | yellow oil | 99.00% |
| 168 | | | | | | |
| Compound | 687.5 | 686.84 | 1-(3,4-dimethoxyphenyl)-N- | A | yellow oil | 91.00% |
| 169 | | | methylmethanamine | | | |
| Compound | 673.4 | 672.82 | (3,4- | A | yellow oil | 95.00% |
| 170 | | | dimethoxyphenyl)methanamine | | | |
| Compound | 657.4 | 656.82 | 1-(3-methoxyphenyl)-N- | C | yellow oil | 100.00% |
| 171 | | | methylmethanamine | | | |
| Compound | 581.4 | 580.72 | 3-aminopropan-1-ol | C | yellow oil | 100.00% |
| 175 | | | | | | |
| Compound | 631.4 | 630.78 | 3-(1H-imidazol-1-yl)propan- | C | yellow gum | 100.00% |
| 176 | | | 1-amine | | | |
| Compound | 642.5 | 641.81 | 3-(pyridin-2-yl)propan-1- | A | yellow oil | 97.00% |
| 177 | | | amine | | | |
| Compound | 609.3 | 608.77 | 4-amino-2-methylbutan-2-ol | A | yellow oil | 100.00% |
| 178 | | | | | | |
| Compound | 604.2 | 603.76 | 5-aminopentanenitrile | A | yellow gum | 100.00% |
| 179 | | | | | | |
| Compound | | 657.83 | N-(3-aminopropyl)methanesulfonamide | | yellow gum | 100.00% |
| 180 | | | | | | |
| Compound | 609.4 | 608.77 | 4-methoxybutan-1-amine | C | yellow oil | 100.00% |
| 181 | | | | | | |
| Compound | 618.5 | 617.78 | 6-aminohexanenitrile | C | yellow oil | 93.00% |
| 182 | | | | | | |

Compound 183:

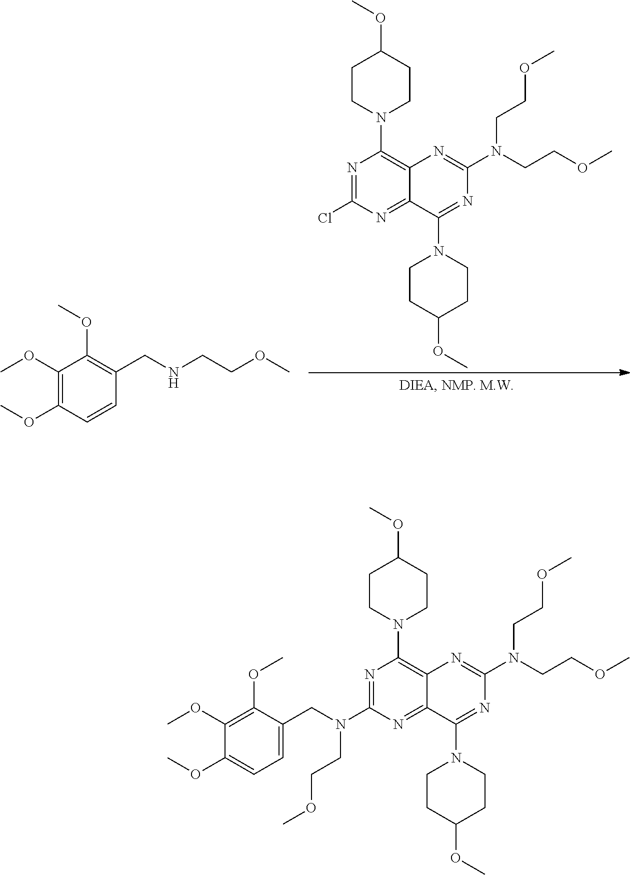

To a solution of intermediate compound 9 (150.0 mg, 286.23 μmol, 1 eq) and intermediate compound 17 (292.31 mg, 1.14 mmol, 4 eq) in NMP (2.0 mL) was added DIEA (147.97 mg, 1.14 mmol, 199.42 μl, 4 eq) in one portion under N2. This reaction mixture was stirred at 200° C. for 3 hr in microwave. The reaction mixture was purified by Prep-HPLC (column: Shim-pack C18 150*25*10 um; mobile phase: [water(0.225% FA)-ACN];B %: 41%-61%, 10 min) to give the sample 183 (43.7 mg, 19.2% yield, 93% purity) as yellow oil. LCMS (ESI position ion) m/z: (M+H)+: 743.5 (calculated: 742.44). ¹H NMR (400 MHz, CHLOROFORM-d) δ=6.88-6.75 (m, 1H), 6.55 (d, J=8.7 Hz, 1H), 4.85 (s, 2H), 4.78-4.45 (m, 4H), 3.99-3.69 (m, 16H), 3.65-3.47 (m, 10H), 3.46-3.25 (m, 16H), 2.03-1.86 (m, 4H), 1.71-1.57 (m, 4H).

Compound 184:

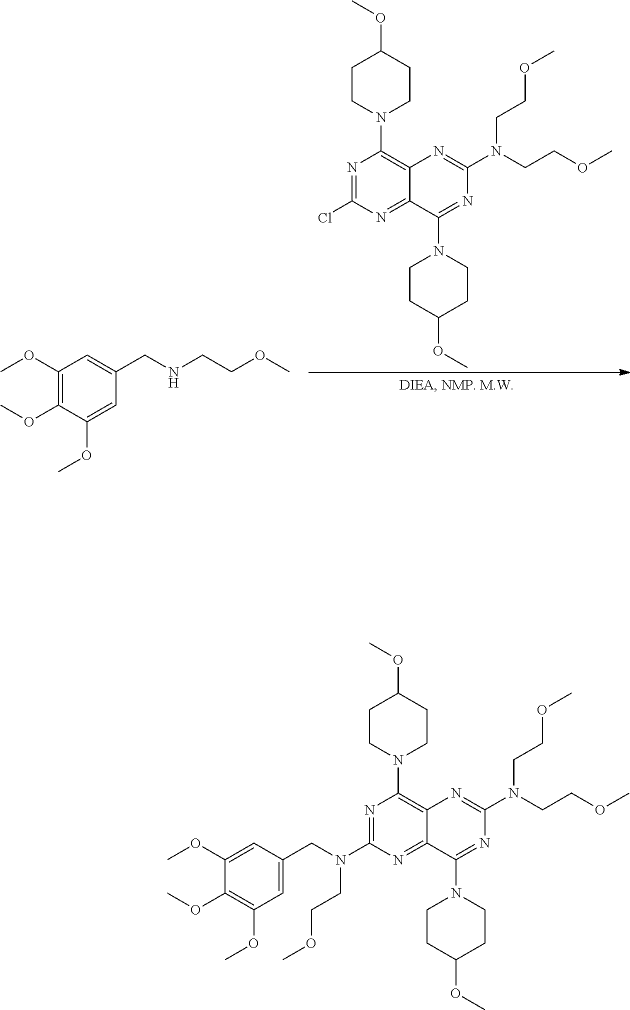

To a solution of intermediate compound 9 (150.0 mg, 286.23 μmol, 1 eq) and intermediate compound 18 (292.31 mg, 1.14 mmol, 4 eq) in NMP (2.0 mL) was added DIEA (147.97 mg, 1.14 mmol, 199.42 μl, 4 eq) in one portion under N2. This reaction mixture was stirred at 200° C. for 6 hr in microwave. The reaction mixture was purified by Prep-HPLC (column: Shim-pack C18 150*25*10 um; mobile phase: [water(0.225% FA)-ACN];B %: 37%-57%, 10 min) to give the Compound 184 (42.3 mg, 19.2% yield, 96.7% purity) as a yellow oil.

LCMS (ESI position ion) m/z: (M+H)+: 743.3 (calculated: 742.44)

¹H NMR (400 MHz, CHLOROFORM-d) δ=6.54 (s, 2H), 4.82 (s, 2H), 4.78-4.56 (m, 4H), 3.89-3.71 (m, 16H), 3.67-3.51 (m, 10H), 3.44-3.29 (m, 16H), 2.06-1.86 (m, 4H), 1.74-1.65 (m, 4H).

Compound 185:

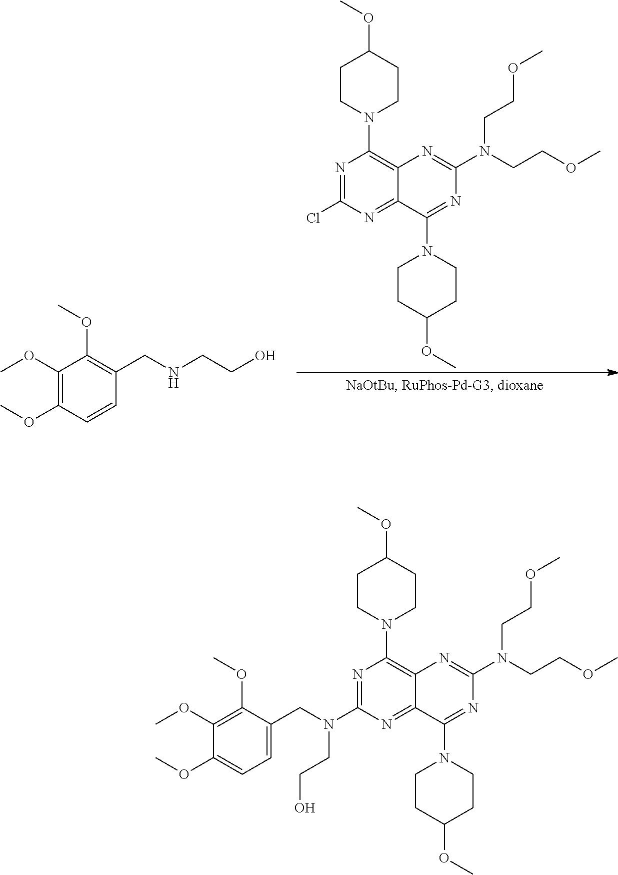

To a solution of intermediate compound 9 (150.0 mg, 286.23 μmol, 1 eq), intermediate compound 19 (276.25 mg, 1.14 mmol, 4 eq) and NaOtBu (82.52 mg, 858.69 μmol, 3 eq) in dioxane (2.0 mL) was added RuPhos Pd G3 (23.94 mg, 28.62 μmol, 0.1 eq) in one portion under N2. This reaction mixture was stirred at 90° C. for 16 hr. The reaction mixture was filtered, and the filtrate was concentrated. The residue was purified by Prep-HPLC (column: Phenomenex Luna C18 150*25 mm*10 um; mobile phase: [water(0.225% FA)-ACN];B %: 30%-60%, 11 min) and then by Prep-HPLC (column: Phenomenex Gemini-NX C18 75*30 mm*3 um; mobile phase: [water(0.1% TFA)-ACN];B %: 40%-50%, 7 min) to give the Compound 185 (32.6 mg, 14.8% yield, 95% purity) as a yellow oil.

LCMS (ESI position ion) m/z: (M+H)+: 729.4 (calculated: 728.42). ¹H NMR (400 MHz, CHLOROFORM-d) δ=6.90 (br d, J=8.0 Hz, 1H), 6.64 (d, J=8.5 Hz, 1H), 4.84 (s, 2H), 3.94-3.84 (m, 14H), 3.82-3.75 (m, 5H), 3.68-3.47 (m, 9H), 3.14-3.76 (m, 14H), 2.12-1.94 (m, 4H), 1.88-1.77 (m, 2H), 1.74-1.63 (m, 2H).

Compound 186:

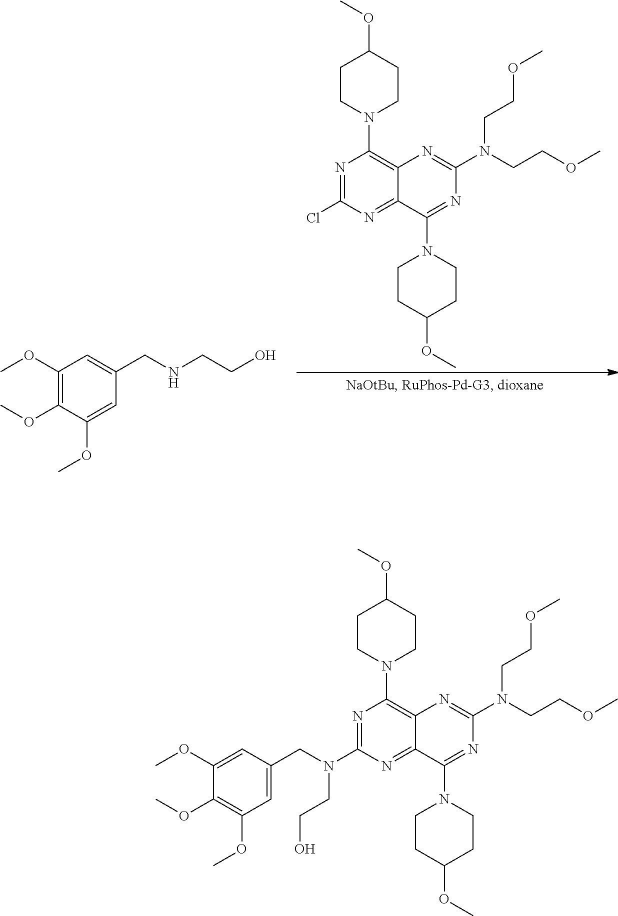

To a solution of intermediate compound 9 (150.0 mg, 286.23 μmol, 1 eq), intermediate compound 20 (276.25 mg, 1.14 mmol, 4 eq) and NaOtBu (82.52 mg, 858.69 μmol, 3 eq) in dioxane (2.0 mL) was added RuPhos Pd G3 (23.94 mg, 28.62 μmol, 0.1 eq) in one portion under N2. This reaction mixture was stirred at 90° C. for 16 hr. The reaction mixture was filtered, and the filtrate was concentrated. The residue was purified by Prep-HPLC (column: Phenomenex Luna C18 150*25 mm*10 um; mobile phase: [water(0.225% FA)-ACN];B %: 28%-58%, 11 min) the Compound 186 (66.6 mg, 31% yield, 97.2% purity) as a yellow oil.

LCMS (ESI position ion) m/z: (M+H)+: 729.3 (calculated: 728.42)

¹H NMR (400 MHz, CHLOROFORM-d) δ=6.57 (s, 2H), 4.80 (s, 2H), 4.74-4.55 (m, 2H), 4.52-4.39 (m, 2H), 3.93-3.65 (m, 19H), 3.63-3.43 (m, 8H), 3.42-3.28 (m, 12H), 2.05-1.89 (m, 4H), 1.78-1.57 (m, 4H).

Compound 195:

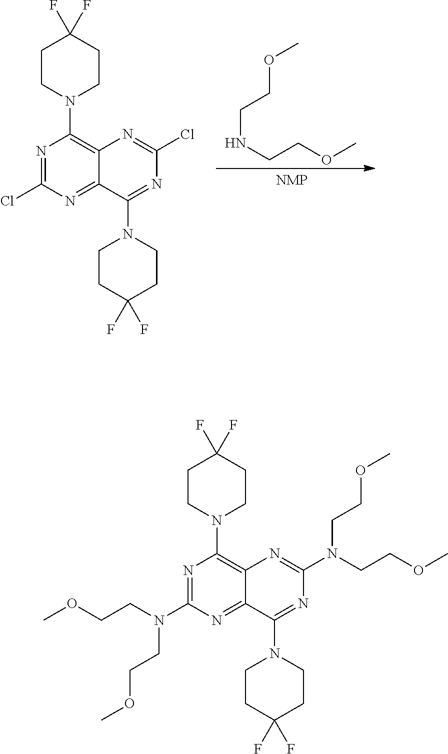

To a solution of intermediate compound 5 (200 mg, 455.34 μmol, 1 eq) and bis(2-methoxyethyl)amine (546 mg, 4.10 mmol, 605.32 μL, 9 eq) in NMP (0.5 mL) was sealed and heated in microwave at 180° C. for 2 hrs. The reaction mixture was quenched by addition water 30 mL at 25° C., and extracted with EtOAc 60 mL (20 mL, 3 times). The combined organic layers were dried over Na₂SO₄, filtered and concentrated under reduced pressure to give a residue. The residue was purified by prep-TLC (SiO2, Petroleum ether/Ethyl acetate=15/1) to give the Compound 195 (32.9 mg, 11.00% yield).

LCMS (ESI position ion) m/z: (M+H)+: 633.3 (calculated: 633.3).

Compound 197:

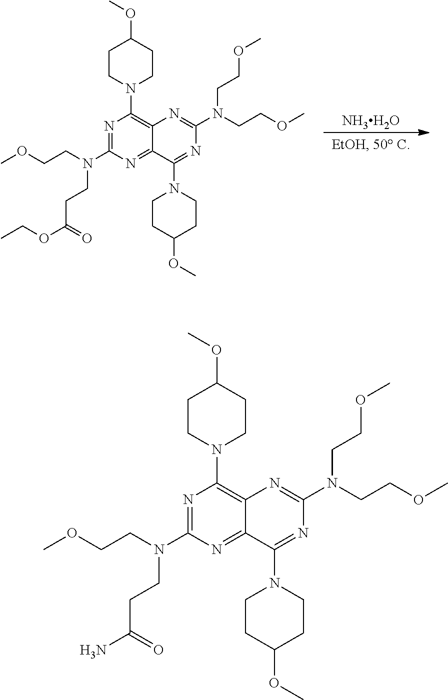

A mixture of Intermediate compound 45 (50 mg, 68.6 μmol, 1 eq) and NH₃.H₂O (280 mg, 2.00 mmol, 307 uL, 25% purity, 29.1 eq) in EtOH (2 mL) was stirred at 50° C. for 16 h. The mixture was concentrated and purified by pre-TLC (SiO₂, Dichloromethane/Methanol=20/1) to give the compound 197 (15 mg, 34% yield) as a yellow solid.

LCMS (ESI position ion) m/z: (M+H)+: 634.3 (calculated: 634.4).

HNMR: (400 MHz, CHLOROFORM-d) δ ppm 6.57-6.56 (m, 1H), 5.19-5.18 (m, 1H), 4.68-4.61 (m, 4H), 3.92 (m, 2H), 3.77 (m, 6H), 3.61-3.57 (m, 10H), 3.40 (m, 2H), 3.39 (s, 6H), 3.36 (s, 9H), 2.62-2.61 (m, 2H), 2.02 (m, 4H), 1.68 (m, 4H).

Compound 201:

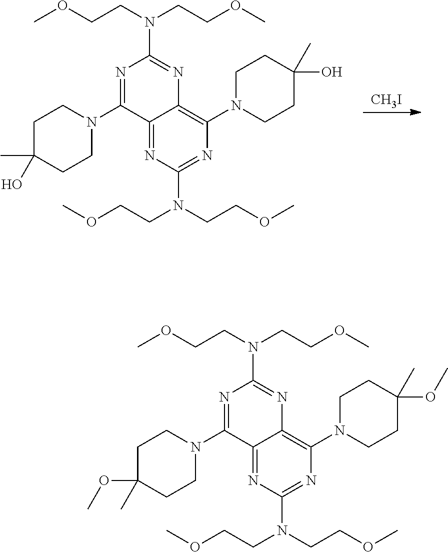

To a solution of Intermediate compound 32 (75 mg, 120.82 μmol, 1 eq) and sodium hydride (15 mg, 375.04 μmol, 60% purity, 3.10 eq) in DMF (0.5 mL) was stirred at 50° C. for 30 min before addition of methyl iodide (52 mg, 366.36 μmol, 22.81 μL, 3.03 eq) at 30° C. The reaction mixture was stirred at 30° C. for 1.5 hrs, quenched by addition of 20 mL of saturated aqueous solution of NH4C1 and extracted with ethyl acetate (10 mL, 2 times). The combined organic layers were dried over Na2SO4, filtered and concentrated under reduced pressure. The residue was purified by prep-HPLC (column: Phenomenex luna C18 150*25 mm*10 um; mobile phase: [water (0.05% HCl)-ACN]; B %: 38%-68%, 10 min) to give the Compound 201 (23.59 mg, 30% yield) as a yellow gum.

LCMS (ESI position ion) m/z: (M+H)+: 649.4 (calculated: 649.4)

Compound 202:

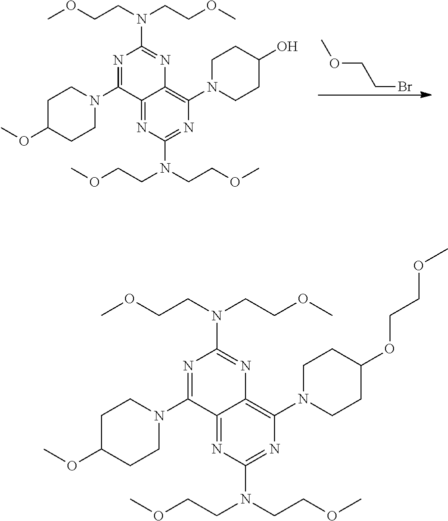

To a solution of Intermediate compound 46 (90 mg, 148.33 μmol, 1 eq) and sodium hydride (9 mg, 225.02 μmol, 60% purity, 1.52 eq) in DMF (0.2 mL) was stirred at 50° C. for 30 min before addition of 1-bromo-2-methoxyethane (27 mg, 194.26 μmol, 18.24 μL, 1.31 eq) at 30° C. The mixture was stirred at 30° C. for 1.5 hrs and quenched by addition of water (30 mL). The aqueous phase was acidified with citric acid to pH=7 and stirred at 16° C. for 30 min and extracted with ethyl acetate (20 mL, 3 times). The combined organic layers were dried over Na2SO4, filtered and concentrated under reduced pressure. The residue was purified by prep-HPLC (column: Phenomenex luna C18 150*25 mm*10 um; mobile phase: [water (0.05% HCl)-ACN]; B %: 30%-60%, 10 min) to give the compound 202 (61.5 mg, 62% yield) as a yellow gum.

LCMS (ESI position ion) m/z: (M+H)+: 665.6 (calculated: 665.4). 1H NMR (400 MHz, CHLOROFORM-d) δ ppm 1.83-1.67 (m, 4H) 2.07-1.96 (m, 4H) 3.33 (s, 12H) 3.36-3.35 (m, 6H) 3.57-3.49 (m, 3H) 3.65-3.58 (m, 10H) 3.68-3.67 (m, 1H) 3.95-3.76 (m, 12H) 4.25-4.03 (m, 4H) 7.46-7.27 (m, 2H)

Compound 231:

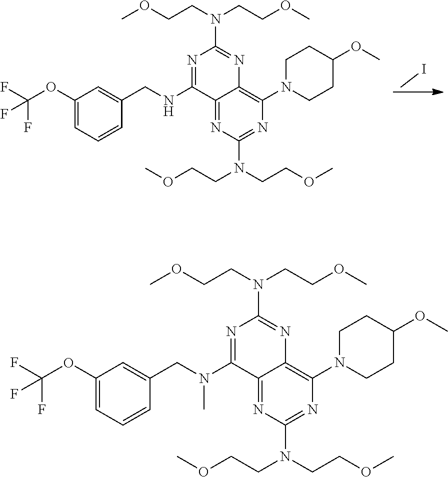

To a solution of Intermediate compound 38 (70 mg, 100.47 μmol, 1 eq) in DMF (2 mL) was cooled to 0° C., and Sodium hydride (4.82 mg, 200.93 μmol, 2 eq) was added, then the reaction was stirred at 0° C. for 20 min, then methyl iodide (42.78 mg, 301.40 μmol, 18.76 μL, 3 eq) was added in one portion, then the reaction was warmed to 15° C. and stirred for 2 hrs. The reaction was quenched by a saturated aqueous solution of NH4C1 (2 mL), diluted by DCM (20 mL) and washed with brine (20 mL, 3 times). The organic layer was dried over Na₂SO₄, concentrated to dryness. The crude was purified by prep-HPLC (column: Waters Xbridge 150*25 mm*5 um; mobile phase: [water (10 mM NH4HCO3)-ACN]; B %: 80%-100%, 5 min) to give the Compound 231 (42.30 mg, 59% yield) as a yellow gum.

LCMS (ESI position ion) m/z: (M+H)+: 711.3 (calculated: 711.4)

Compound 232:

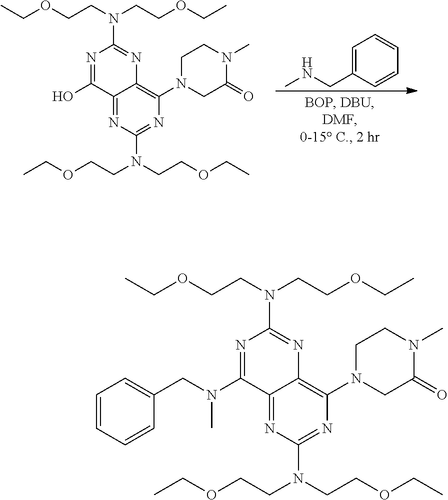

To a solution of N-methyl-1-phenylmethanamine (31.41 mg, 259.20 μmol) and Intermediate compound 37 (50 mg, 86.40 μmol) in DMF (0.5 mL) was added 1H-Benzotriazol-1-yloxytris (dimethylamino) phosphonium hexafluorophosphate (57.32 mg, 129.60 μmol) and 1,8-diazabicyclo [5.4.0]-7-undecene (19.73 mg, 129.60 μmol) at 20° C. The mixture was stirred at 20° C. for 2 hr. The reaction mixture was diluted by water (10 mL) and extracted by ethyl acetate (5 mL, 2 times). The organic phase was combined, washed with brine (5 mL), dried over sodium sulfate, filtered and concentrated under reduced pressure. The residue was purified by prep-HPLC (Method B) to give the Compound 232 (40.2 mg, 67%) as yellow oil.

LCMS (ESI position ion) m/z: (M+H)+: 682.5 (calculated: 682.4). ¹H NMR (ET30779-50-P1N1, CDCl₃-d 400 MHz) δ ppm 1.07-1.23 (m, 12H) 3.03 (s, 3H) 3.29-4.18 (m, 25H) 4.13 (br s, 4H) 4.31 (s, 3H) 5.53 (br s, 2H) 7.24 (br d, J=7.28 Hz, 2H) 7.29-7.41 (m, 3H)

Compound 240:

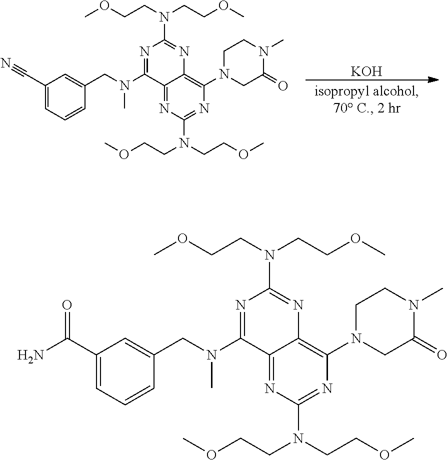 [SMILES: COCCN(CCOC)c1nc(N2CCN(C)C(=O)C2)c2nc(N(CCOC)CCOC)nc(N(C)Cc3cccc(C#N)c3)c2n1 ; "KOH" ; COCCN(CCOC)c1nc(N2CCN(C)C(=O)C2)c2nc(N(CCOC)CCOC)nc(N(C)Cc3cccc(C(N)=O)c3)c2n1]

To a solution of Compound 237 (90 mg, 138.30 μmol) in isopropyl alcohol (1 mL) was added KOH (23.28 mg, 414.89 μmol) at 20° C. The reaction mixture was stirred at 70° C. for 2 hr. The reaction mixture was quenched with water (10 mL), extracted with ethyl acetate (5 mL, 2 times). The combined organic phase was washed with brine (5 mL, 2 times), dried over Na2SO4, filtered and concentrated under reduced pressure. The residue was purified by prep-HPLC (Method A) to give the Compound 240 (20 mg, yield 22%) as yellow oil.

LCMS (ESI position ion) m/z: (M+H)+: 669.3 (calculated: 669.4)

Compound 249:

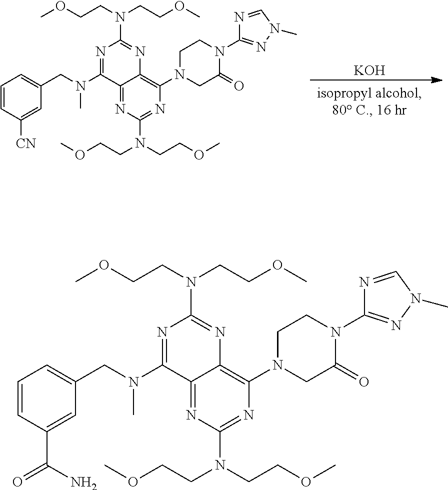 [SMILES: COCCN(CCOC)c1nc(N2CCN(c3ncn(C)n3)C(=O)C2)c2nc(N(CCOC)CCOC)nc(N(C)Cc3cccc(C#N)c3)c2n1 ; "KOH" ; COCCN(CCOC)c1nc(N2CCN(c3ncn(C)n3)C(=O)C2)c2nc(N(CCOC)CCOC)nc(N(C)Cc3cccc(C(N)=O)c3)c2n1]

To a solution of Compound 244 (90 mg, 127.87 μmol) in isopropyl alcohol (1 mL) was added KOH (21.52 mg, 383.61 μmol) at 20° C. The reaction mixture was stirred at 80° C. for 16 hr. The reaction mixture was worked up and purified by prep-TLC (SiO₂, eluted with ethyl acetate/methanol=5/1, R_{f}=0.44) to give crude product. The crude product was purified by prep-HPLC (Method A) to give the Compound 249 (7.2 mg, yield 8%) as yellow solid. LCMS (ESI position ion) m/z: (M+H)+: 722.3 (calculated: 722.4).

¹H NMR: (ET28588-1149-P1N1, CDCl₃-d, 400 MHz) δ=7.69-7.80 (m, 3H), 7.39-7.48 (m, 2H), 5.36-5.57 (m, 2H), 4.17-4.36 (m, 3H), 3.69-3.85 (m, 8H), 3.57 (br s, 13H), 3.22-3.46 (m, 20H).

General Procedure III

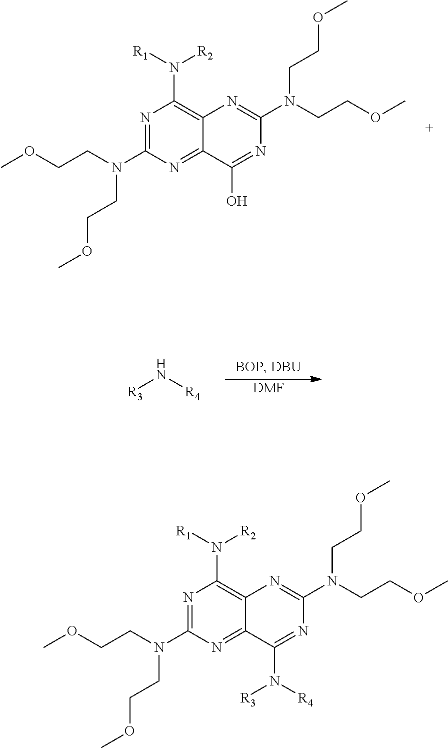

To a solution of required intermediate compound (30 mg, 95.49 μmol, 1 eq), BOP (1.3 eq) and DBU (1.5 eq) in DMF (0.4 mL) was stirred at 20° C. for 1 hr before addition of the required amine (3.00 eq) in DMF (0.1 mL). The reaction mixture was stirred at 20° C. for 14 hr. The reaction mixture was diluted with water 5 mL and extracted with ethyl acetate (2 mL, 3 times). The combined organic layers were dried over Na₂SO₄, filtered and concentrated under reduced pressure. The residue was purified by preparative HPLC following the purification method A, B or C to give the desired Compound as indicated in the Table 4.

Compounds that were obtained according to General Procedure III is presented in Table 4.

TABLE 4

| | LCMS (ESI | | | | | | |
|---|---|---|---|---|---|---|---|
| | position ion) | Calculated | Intermediate | | Method of | | |
| Compound | m/z: (M + H)+ | Molar Mass | compound | Amines | purification | Appearance | Purity |
| Compound | 629.3 | 628.727 | 23 | 5,6,7,8-tetrahydro- | A | yellow | 100.00% |
| 5 | | | | [1,2,4]triazolo[1,5- | | solid | |
| | | | | a]pyrazine | | | |
| Compound | 650.2 | 649.768 | 25 | 5,6,7,8-tetrahydro- | C | yellow | 100.00% |
| 6 | | | | [1,2,4]triazolo[1,5- | | solid | |
| | | | | a]pyrazine | | | |
| Compound | 633.4 | 632.777 | 25 | phenylmethanamine | C | yellow | 95.00% |
| 7 | | | | | | solid | |
| Compound | 599.5 | 598.761 | 25 | N-methylpropan-1-amine | C | yellow | 96.00% |
| 8 | | | | | | solid | |
| Compound | 639.4 | 638.726 | 27 | 5,6,7,8-tetrahydro- | A | yellow | 98.00% |
| 9 | | | | [1,2,4]triazolo[1,5- | | solid | |
| | | | | a]pyrazine | | | |
| Compound | 588.3 | 587.719 | 27 | N-methylpropan-1-amine | A | yellow | 100.00% |
| 10 | | | | | | solid | |
| Compound | 619.3 | 618.729 | 23 | 1-methylpiperazin-2-one | A | yellow | 100.00% |
| 187 | | | | | | solid | |
| Compound | 640.2 | 639.769 | 23 | thiomorpholine 1,1-dioxide | A | yellow | 100.00% |
| 188 | | | | | | solid | |
| Compound | 646.3 | 645.675 | 23 | 3-(trifluoromethyl)azetidin- | A | yellow | 100.00% |
| 189 | | | | 3-ol | | solid | |
| Compound | 612.3 | 611.737 | 23 | phenylmethanamine | C | yellow | 100.00% |
| 190 | | | | | | solid | |
| Compound | 578.4 | 577.72 | 23 | N-methylpropan-1-amine | C | yellow | 100.00% |
| 191 | | | | | | solid | |
| Compound | 667.4 | 666.715 | 25 | 3-(trifluoromethyl)azetidin- | C | yellow | 100.00% |
| 192 | | | | 3-ol | | solid | |
| Compound | 656.3 | 655.673 | 27 | 3-(trifluoromethyl)azetidin- | A | yellow | 95.00% |
| 193 | | | | 3-ol | | solid | |
| Compound | 622.3 | 621.735 | 27 | phenylmethanamine | A | yellow | 96.00% |
| 194 | | | | | | solid | |
| Compound | 673.3 | 673.4 | 36 | 4-methoxypiperidine | A | yellow gum | 99.00% |
| 196 | | | | | | | |
| Compound | 634.3 | 634.4 | 16 | 1-ethylpiperazin-2-one | C | yellow | 94.00% |
| 198 | | | | | | solid | |
| Compound | 676.6 | 676.4 | 23 | 4-methoxypiperidine | A | yellow | 100.00% |
| 199 | | | | | | solid | |
| Compound | 627.2 | 627.4 | 16 | 4,4-difluoropiperidine | C | yellow | 96.00% |
| 200 | | | | | | solid | |
| Compound | 655.2 | 655.4 | 23 | 3-(aminomethyl)benzamide | A | yellow gum | 98.00% |
| 203 | | | | | | | |
| Compound | 691.4 | 691.3 | 23 | 3- | A | yellow | 79.00% |
| 204 | | | | (aminomethyl)benzenesulfonamide | | solid | |
| Compound | 637.2 | 637.3 | 23 | 3-(aminomethyl)benzonitrile | B | yellow gum | 96.00% |
| 205 | | | | | | | |
| Compound | 626.4 | 626.3 | 23 | 4,4-difluoropiperidine | A | yellow | 100.00% |
| 206 | | | | | | solid | |
| Compound | 630.3 | 630.3 | 23 | (3-fluorophenyl)methanamine | A | yellow | 100.00% |
| 207 | | | | | | solid | |
| Compound | 646.3 | 646.3 | 23 | (3-chlorophenyl)methanamine | C | yellow | 100.00% |
| 208 | | | | | | solid | |
| Compound | 642.4 | 642.4 | 23 | (3-methoxyphenyl)methanamine | C | yellow | 98.00% |
| 209 | | | | | | solid | |
| Compound | 696.2 | 696.3 | 23 | (3-(trifluoro- | A | yellow gum | 94.00% |
| 210 | | | | methoxy)phenyl)methanamine | | | |
| Compound | 655.2 | 655.3 | 23 | 5-(aminomethyl)-2- | C | yellow | 94.00% |
| 211 | | | | fluorobenzonitrile | | solid | |
| Compound | 690.5 | 690.3 | 23 | (3-(methyl- | C | yellow | 100.00% |
| 212 | | | | sulfonyl)phenyl)methanamine | | solid | |
| Compound | 648.2 | 648.3 | 23 | (3,4- | A | yellow gum | 100.00% |
| 213 | | | | difluorophenyl)methanamine | | | |
| Compound | 564.3 | 564.4 | 23 | propan-1-amine | A | yellow | 99.00% |
| 214 | | | | | | solid | |
| Compound | 578.5 | 578.4 | 23 | 2-methylpropan-1-amine | A | yellow | 100.00% |
| 215 | | | | | | solid | |
| Compound | 672.4 | 672.4 | 36 | 1-methylpiperazin-2-one | B | yellow oil | 94.00% |
| 216 | | | | | | | |
| Compound | 693.4 | 693.4 | 36 | thiomorpholine 1,1-dioxide | A | yellow | 98.00% |
| 217 | | | | | | solid | |
| Compound | 617.4 | 617.4 | 36 | propan-1-amine | A | yellow oil | 100.00% |
| 218 | | | | | | | |
| Compound | 631.3 | 631.4 | 36 | 2-methylpropan-1-amine | A | yellow | 93.00% |
| 219 | | | | | | solid | |
| Compound | 683.3 | 683.4 | 36 | (3-fluorophenyl)methanamine | A | yellow oil | 95.00% |
| 220 | | | | | | | |
| Compound | 699.4 | 699.4 | 36 | (3- | A | yellow oil | 98.00% |
| 221 | | | | chlorophenyl)methanamine | | | |
| Compound | 695.3 | 695.4 | 36 | (3- | A | yellow oil | 95.00% |
| 222 | | | | methoxyphenyl)methanamine | | | |
| Compound | 749.4 | 749.4 | 36 | (3-(trifluoro- | A | yellow | 100.00% |
| 223 | | | | methoxy)phenyl)methanamine | | solid | |
| Compound | 690.4 | 690.4 | 36 | 3-(aminomethyl)benzonitrile | C | yellow | 100.00% |
| 224 | | | | | | solid | |
| Compound | 708.3 | 708.4 | 36 | 5-(aminomethyl)-2- | A | yellow | 90.00% |
| 225 | | | | fluorobenzonitrile | | solid | |
| Compound | 743.4 | 743.4 | 36 | (3-(methyl- | C | yellow oil | 79.00% |
| 226 | | | | sulfonyl)phenyl)methanamine | | | |
| Compound | 701.4 | 701.4 | 36 | (3,4- | A | yellow oil | 86.00% |
| 227 | | | | difluorophenyl)methanamine | | | |
| Compound | 744.4 | 744.4 | 36 | 3-(amino- | A | yellow | 74.00% |
| 228 | | | | methyl)benzenesulfonamide | | solid | |
| Compound | 708.4 | 708.4 | 36 | 3-(aminomethyl)benzamide | A | yellow | 60.00% |
| 229 | | | | | | solid | |
| Compound | 626.2 | 626.4 | 23 | N-methyl-1-phenylmethanamine | A | yellow gum | 100.00% |
| 230 | | | | | | | |
| Compound | 644.3 | 644.4 | 23 | 1-(3-fluorophenyl)-N- | A | yellow oil | 94.00% |
| 233 | | | | methylmethanamine | | | |
| Compound | 660.3 | 660.3 | 23 | 1-(3-chlorophenyl)-N- | A | yellow | 100.00% |
| 234 | | | | methylmethanamine | | solid | |
| Compound | 656.3 | 656.4 | 23 | 1-(3-methoxyphenyl)-N- | A | yellow oil | 99.00% |
| 235 | | | | methylmethanamine | | | |
| Compound | 710.4 | 710.4 | 23 | Intermediate compound 39 | A | yellow | 100.00% |
| 236 | | | | | | solid | |
| Compound | 651.3 | 651.4 | 23 | 3-((methyl- | C | yellow | 100.00% |
| 237 | | | | amino)methyl)benzonitrile | | solid | |
| Compound | 669.4 | 669.4 | 23 | Intermediate compound 40 | C | yellow | 99.00% |
| 238 | | | | | | solid | |
| Compound | 662.3 | 662.3 | 23 | 1-(3,4-difluorophenyl)- | C | yellow oil | 98.00% |
| 239 | | | | N-methylmethanamine | | | |
| Compound | 697.3 | 697.4 | 36 | 1-(3-fluorophenyl)-N- | B | yellow oil | 97.00% |
| 241 | | | | methylmethanamine | | | |
| Compound | 713.3 | 713.3 | 36 | 1-(3-chlorophenyl)- | B | yellow oil | 98.00% |
| 242 | | | | N-methylmethanamine | | | |
| Compound | 709.4 | 709.4 | 36 | 1-(3-methoxyphenyl)-N- | A | yellow | 98.00% |
| 243 | | | | methylmethanamine | | solid | |
| Compound | 704.4 | 704.4 | 36 | 3-((methylamino)methyl)benzonitrile | A | yellow | 100.00% |
| 244 | | | | | | solid | |
| Compound | 722.4 | 722.4 | 36 | Intermediate compound 40 | C | yellow | 98.00% |
| 245 | | | | | | solid | |
| Compound | 757.4 | 757.4 | 36 | Intermediate compound 41 | C | yellow | 100.00% |
| 246 | | | | | | solid | |
| Compound | 715.4 | 715.4 | 36 | Intermediate compound 43 | C | yellow | 100.00% |
| 247 | | | | | | solid | |
| Compound | 758.4 | 758.4 | 36 | Intermediate compound 42 | A | yellow | 98.00% |
| 248 | | | | | | solid | |
| Compound | 628.2 | 628.4 | 23 | 5,6,7,8- | A | yellow oil | 91.00% |
| 250 | | | | tetrahydroimidazo[1,5- | | | |
| | | | | a]pyrazine | | | |
| Compound | 629.3 | 629.4 | 23 | 4,5,6,7-tetrahydro- | C | yellow | 100.00% |
| 251 | | | | [1,2,3]triazolo[1,5- | | solid | |
| | | | | a]pyrazine | | | |
| Compound | 642.3 | 642.4 | 23 | 2-methyl-4,5,6,7-tetrahydro- | C | yellow oil | 95.00% |
| 252 | | | | 2H-pyrazolo[4,3- | | | |
| | | | | c]pyridinedihydrochloride | | | |
| Compound | 642.4 | 642.4 | 23 | 1-methyl-4,5,6,7-tetrahydro- | C | yellow | 91.00% |
| 253 | | | | 1H-imidazo[4,5-c]pyridine | | solid | |
| Compound | 629.3 | 629.3 | 23 | 4,5,6,7- | C | yellow | 94.00% |
| 254 | | | | tetrahydroisoxazolo[4,3- | | solid | |
| | | | | c]pyridine | | | |
| Compound | 642.4 | 642.4 | 23 | 1-methyl-4,5,6,7-tetrahydro- | C | yellow | 100.00% |
| 255 | | | | 1H-pyrazolo[4,3-c]pyridine | | solid | |
| Compound | 643.4 | 643.4 | 23 | 2-methyl-4,5,6,7- | A | yellow | 98.00% |
| 256 | | | | tetrahydrooxazolo[4,5- | | solid | |
| | | | | c]pyridine | | | |
| Compound | 628.4 | 628.4 | 23 | 5,6,7,8-tetrahydroimidazo[1,2- | C | yellow | 93.00% |
| 257 | | | | a]pyrazine | | solid | |
| Compound | 627.3 | 627.4 | 23 | 1,2,3,4- | C | yellow | 90.00% |
| 258 | | | | tetrahydropyrrolo[1,2- | | solid | |
| | | | | a]pyrazine | | | |
| Compound | 627.4 | 627.4 | 23 | 4,5,6,7-tetrahydro-2H- | C | yellow | 96.00% |
| 259 | | | | pyrazolo[4,3-c]pyridine | | solid | |
| Compound | 628.3 | 628.4 | 23 | 4,5,6,7- | C | yellow | 92.00% |
| 260 | | | | tetrahydropyrazolo[1,5- | | solid | |
| | | | | a]pyrazine | | | |
| Compound | 629.4 | 629.3 | 23 | 4,5,6,7- | A | yellow | 99.00% |
| 261 | | | | tetrahydroisoxazolo[4,5- | | solid | |
| | | | | c]pyridine | | | |

General Procedure IV

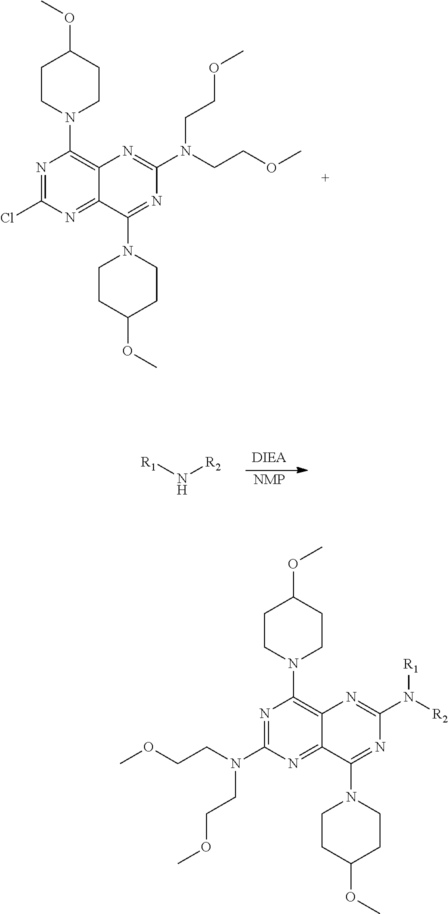

To a solution of intermediate compound 9 (100 mg, 190.82 μmol, 1 eq), DIEA (62 mg, 479.73 μmol, 83.56 μl, 2.51 eq) and the corresponding amine (9.00 eq) in NMP (1 mL) was sealed and heated in microwave at 180° C. for 2 hr. The reaction mixture was diluted with water 5 mL and extracted with ethyl acetate 6 mL (2 mL, 3 times). The combined organic layers were dried over Na2SO4, filtered and concentrated under reduced pressure to give a residue.

Compounds 30 to 32 and 132 to 134 were obtained according to General Procedure IV:

Compound 30: The amine used was the 1-(methylsulfonyl)piperazine (9 eq, 1.717 mmol, 283 mg). The residue was purified by prep-HPLC (column: 3_Phenomenex Luna C18 75*30 mm*3 um; mobile phase: [water(0.05% HCl)-ACN];B %: 29%-49%, 6.5 min) to give Compound 30 (48.8 mg, 38% yield, 96.5% purity) as a yellow oil.

LCMS (ESI position ion) m/z: (M+H)+: 652.2 (calculated: 651.35)

Compound 31: The amine used was the ethyl piperazine-1-carboxylate (9 eq, 1.717 mmol, 272 mg). The residue was purified by prep-HPLC (column: 3_Phenomenex Luna C18 75*30 mm*3 um; mobile phase: [water(0.05% HCl)-ACN]; B %: 29%-49%, 6.5 min) to give Compound 31 (68 mg, 55% yield, 100% purity) as a yellow oil. LCMS (ESI position ion) m/z: (M+H)+: 646.2 (calculated: 645.40)

Compound 32: The amine used was the 2-(1H-pyrazol-1-yl)ethan-1-amine (9 eq, 1.717 mmol, 191 mg). The residue was purified by pre-HPLC (column: 3_Phenomenex Luna C18 75*30 mm*3 um; mobile phase: [water (0.05% HCl)-ACN]; B %: 28%-48%, 6.5 min) to give Compound 32 (16.8 mg, 14.5% yield, 98% purity) as a yellow oil.

LCMS (ESI position ion) m/z: (M+H)+: 599.3 (calculated: 598.37)

Compound 132: The amine used was the N-ethyl-2-(pyrrolidin-1-yl)ethan-1-amine (9 eq, 1.717 mmol, 244 mg). The residue was purified by prep-HPLC (column: Phenomenex luna C18 150*25 mm*10 um; mobile phase: [water (0.05% HCl)-ACN]; B %: 12%-42%, 10 min) to give the Compound 132 (90.9 mg, 76% yield, 100% purity) as a yellow oil.

LCMS (ESI position ion) m/z: (M+H)+: 630.4 (calculated: 629.44)

Compound 133: The amine used was the N-ethyl-2-(piperidin-1-yl)ethan-1-amine (9 eq, 1.717 mmol, 269 mg). The residue was purified by prep-HPLC (column: Phenomenex luna C18 150*25 mm*10 um; mobile phase: [water (0.05% HCl)-ACN]; B %: 17%-47%, 10 min)) to give the Compound 133 (58.2 mg, 48% yield, 100% purity) as a yellow oil. LCMS (ESI position ion) m/z: (M+H)+: 644.5 (calculated: 643.45)

Compound 134: The amine used was the 3-(1H-imidazol-1-yl)propan-1-amine (9 eq, 1.717 mmol, 215 mg). The residue was purified by prep-HPLC (column: 3_Phenomenex Luna C18 75*30 mm*3 um; mobile phase: [water (0.05% HCl)-ACN]; B %: 17%-37%, 7 min) to give the Compound 134 (45.4 mg, 39% yield, 98% purity) as a yellow oil. LCMS (ESI position ion) m/z: (M+H)+: 613.2 (612.39)

General Procedure V

To a solution of either the intermediate compound 52 or intermediate compound 53 (50 mg, 1 eq) in NMP (1.5 mL) were added amine (8 eq) and DIEA (5 eq). The mixture was stirred at 230° C. for 2 hr. LCMS showed starting material was consumed completely and desired mass was detected. The reaction mixture was filtered. The filtrate was evaporated.

Compounds 267 to 272 and 275, 276, 278, 279, 281 to 284, 287, 288, 292 to 298, 302, 303, 327, 333 were obtained according to General Procedure V:

Compound 267:

The intermediate compound 52 and the N-ethyl-2-(pyrrolidin-1-yl)ethan-1-amine (9 eq, 1.717 mmol, 283 mg) was used. The residue was purified by prep-HPLC (column: Phenomenex luna C18 150*25 mm*10 um; mobile phase: [water(0.225% FA)-ACN];B %: 13%-43%, 10 min) to give the compound 267 (48.8 mg, 38% yield) as a yellow oil.

LCMS (ESI position ion) m/z: (M+H)+: 629.3 (calculated: 628.42)

Compound 268:

The intermediate compound 52 and the 2-methoxy-N-(3,4,5-trimethoxybenzyl)ethan-1-amine (9 eq, 1.717 mmol, 283 mg) was used. The residue was purified by prep-HPLC (column: Phenomenex Gemini-NX C18 75*30 mm*3 um; mobile phase: [water(10 mM NH4HCO3)-ACN];B %: 45%-75%, 8 min).

LCMS (ESI position ion) m/z: (M+H)+: 742.4 (calculated: 741.41)

Compound 269:

The intermediate compound 53 and the 2-methoxy-N-(3,4,5-trimethoxybenzyl)ethan-1-amine (9 eq, 1.717 mmol, 283 mg) was used. The residue was purified by prep-HPLC (column: Phenomenex Gemini-NX C18 75*30 mm*3 um; mobile phase: [water(10 mM NH4HCO3)-ACN];B %: 45%-75%, 8 min).

LCMS (ESI position ion) m/z: (M+H)+: 742.4 (calculated: 741.41)

Compound 270:

The intermediate compound 52 and the 2,2′-azanediylbis(ethan-1-ol) (9 eq, 1.717 mmol, 283 mg) was used. The residue was purified by prep-HPLC (column: Phenomenex luna C18 150*25 mm*10 um; mobilephase: [water(0.225% FA)-ACN];B %: 12%-42%, 10 min).

LCMS (ESI position ion) m/z: (M+H)+: 592.2 (calculated: 591.35)

Compound 271:

The intermediate compound 53 and the 2,2′-azanediylbis(ethan-1-ol) (9 eq, 1.717 mmol, 283 mg) was used. The residue was purified by prep-HPLC (column: Phenomenex luna C18 150*25 mm*10 um; mobilephase: [water(0.225% FA)-ACN];B %: 12%-42%, 10 min).

LCMS (ESI position ion) m/z: (M+H)+: 592.3 (calculated: 591.35)

Compound 272:

The intermediate compound 52 and the N-ethyl-2-(piperidin-1-yl)ethan-1-amine (9 eq, 1.717 mmol, 283 mg) was used. The residue was purified by prep-HPLC (column: Phenomenex luna C18 150*25 mm*10 um; mobile phase: [water(0.225% FA)-ACN];B %: 12%-42%, 10 min).

LCMS (ESI position ion) m/z: (M+H)+: 643.3 (calculated: 642.43)

Compound 275:

The intermediate compound 52 and the 2-((2-methoxyethyl)amino)ethan-1-ol (9 eq, 1.717 mmol, 283 mg) was used. The residue was purified by prep-HPLC (column: Waters Xbridge 150*25 mm*5 um; mobile phase: [water(10 mM NH4HCO3)-ACN]; B %: 37%-67%, 10 min).

LCMS (ESI position ion) m/z: (M+H)+: 606.2 (calculated: 605.36)

Compound 276:

The intermediate compound 53 and the 2-((2-methoxyethyl)amino)ethan-1-ol (9 eq, 1.717 mmol, 283 mg) was used. The residue was purified by prep-HPLC (column: Phenomenex Gemini-NX C18 75*30 mm*3 um; mobile phase: [water(0.225% FA)-ACN];B %: 15%-45%, 7 min).

LCMS (ESI position ion) m/z: (M+H)+: 606.4 (calculated: 605.36)

Compound 278:

The intermediate compound 53 and the piperazin-2-one (9 eq, 1.717 mmol, 283 mg) was used. The residue was purified by prep-HPLC (column: Phenomenex Gemini-NX C18 75*30 mm*3 um; mobile phase: [water(0.225% FA)-ACN];B %: 15%-45%, 7 min).

LCMS (ESI position ion) m/z: (M+H)+: 587.3 (calculated: 586.33)

Compound 279:

The intermediate compound 53 and the 1-(methylsulfonyl)piperazine (9 eq, 1.717 mmol, 283 mg) was used. The residue was purified by prep-HPLC (column: Phenomenex luna C18 150*25 mm*10 um; mobilephase: [water(0.225% FA)-ACN];B %: 26%-57%, 10 min).

LCMS (ESI position ion) m/z: (M+H)+: 651.3 (calculated: 650.33)

Compound 281:

The intermediate compound 53 and the 1-(piperazin-1-yl)ethan-1-one (9 eq, 1.717 mmol, 283 mg) was used. The residue was purified by prep-HPLC (column: Phenomenex luna C18 150*25 mm*10 um; mobilephase: [water(0.225% FA)-ACN];B %: 21%-51%, 10 min).

LCMS (ESI position ion) m/z: (M+H)+: 615.3 (calculated: 614.36)

Compound 282:

The intermediate compound 52 and the 5,6,7,8-tetrahydro-[1,2,4]triazolo[1,5-a]pyrazine (9 eq, 1.717 mmol, 283 mg) was used. The residue was purified by prep-HPLC (column: Phenomenex Gemini-NX C18 75*30 mm*3 um; mobile phase: [water(0.225% FA)-ACN];B %: 22%-52%, 7 min).

LCMS (ESI position ion) m/z: (M+H)+: 611.2 (calculated: 610.34)

Compound 283:

The intermediate compound 53 and the 5,6,7,8-tetrahydro-[1,2,4]triazolo[1,5-a]pyrazine (9 eq, 1.717 mmol, 283 mg) was used. The residue was purified by prep-HPLC (column: Phenomenex Gemini-NX C18 75*30 mm*3 um; mobile phase: [water(0.225% FA)-ACN];B %: 25%-55%, 7 min).

LCMS (ESI position ion) m/z: (M+H)+: 611.3 (calculated: 610.34)

Compound 284:

The intermediate compound 53 and the methyl piperazine-1-carboxylate (9 eq, 1.717 mmol, 283 mg) was used. The residue was purified by prep-HPLC (column: Phenomenex Gemini-NX C18 75*30 mm*3 um; mobile phase: [water(0.225% FA)-ACN]; B %: 20%-50%, 7 min).

LCMS (ESI position ion) m/z: (M+H)+: 631.3 (calculated: 630.36)

Compound 287:

The intermediate compound 52 and the 4,5,6,7-tetrahydro-[1,2,3]triazolo[1,5-a]pyrazine (9 eq, 1.717 mmol, 283 mg) was used. The residue was purified by prep-HPLC (column: Waters Xbridge 150*25 mm*5 um; mobile phase: [water(10 mM NH4HCO3)-ACN];B %: 34%-64%, 10 min).

LCMS (ESI position ion) m/z: (M+H)+: 611.2 (calculated: 610.34)

Compound 288:

The intermediate compound 53 and the 4,5,6,7-tetrahydro-[1,2,3]triazolo[1,5-a]pyrazine (9 eq, 1.717 mmol, 283 mg) was used. The residue was purified by prep-HPLC (column: Phenomenex luna C18 150*25 mm*10 um; mobile phase: [water(0.225% FA)-ACN];B %: 22%-52%, 10 min).

LCMS (ESI position ion) m/z: (M+H)+: 611.3 (calculated: 610.34)

Compound 292:

The intermediate compound 53 and the 4-(methylsulfonyl)piperidine (9 eq, 1.717 mmol, 283 mg) was used. The residue was purified by prep-HPLC (column: Phenomenex luna C18 150*25 mm*10 um; mobilephase: [water(0.225% FA)-ACN];B %: 21%-51%, 10 min).

LCMS (ESI position ion) m/z: (M+H)+: 650.3 (calculated: 649.33)

Compound 293:

The intermediate compound 52 (50 mg, 95.60 umol, 1 eq) intermediate compound 75 (160.79 mg, 717.00 umol, 50.90 uL, 7.5 eq) were used. The residue was purified by prep-HPLC (column: Phenomenex Gemini-NX C18 75*30 mm*3 um; mobile phase: [water(0.225% FA)-ACN];B %: 25%-45%, 7 min) followed by prep-HPLC (column: Waters Xbridge 150*25 mm*5 um; mobile phase [water(0.225% FA)-ACN];B %: 13%-43%, 10 min) to give the compound 293 (18.3 mg, 28% yield,) as yellow gum.

LCMS (ESI position ion) m/z: (M+H)+: 665.3 (calculated: 664.39)

Compound 294:

The intermediate compound 53 (50 mg, 95.60 umol, 1 eq) intermediate compound 75 (160.79 mg, 717.00 umol, 50.90 uL, 7.5 eq) were used. The residue was purified by prep-HPLC (column: Phenomenex Gemini-NX C18 75*30 mm*3 um; mobile phase: [water(0.225% FA)-ACN];B %: 25%-45%, 7 min) followed by prep-HPLC (column: Waters Xbridge 150*25 mm*5 um; mobile phase: [water(10 mM NH4HCO3)-ACN];B %: 43%-73%, 10 min) to give the compound 294 (9.6 mg, 13.81 umol, 14% yield) as yellow solid.

LCMS (ESI position ion) m/z: (M+H)+: 665.4 (calculated: 664.39)

Compound 295:

The intermediate compound 52 and the 5,6,7,8-tetrahydroimidazo[1,2-a]pyrazine (9 eq, 1.717 mmol, 283 mg) was used. The residue was purified by prep-HPLC (column: Waters Xbridge 150*25 mm*5 um; mobile phase: [water(10 mM NH4HCO3)-ACN]; B %: 38%-68%, 10 min).

LCMS (ESI position ion) m/z: (M+H)+: 610.2 (calculated: 609.35)

Compound 296:

The intermediate compound 53 and the 5,6,7,8-tetrahydroimidazo[1,2-a]pyrazine (9 eq, 1.717 mmol, 283 mg) was used. The residue was purified by prep-HPLC (column: Waters Xbridge 150*25 mm*5 um; mobile phase: [water(10 mM NH4HCO3)-ACN]; B %: 38%-68%, 10 min).

LCMS (ESI position ion) m/z: (M+H)+: 610.3 (calculated: 609.35)

Compound 297:

The intermediate compound 52 and the 5,6,7,8-tetrahydroimidazo[1,5-a]pyrazine (9 eq, 1.717 mmol, 283 mg) was used. The residue was purified by prep-HPLC (column: Waters Xbridge 150*25 mm*5 um; mobile phase: [water(10 mM NH4HCO3)-ACN]; B %: 34%-64%, 10 min).

LCMS (ESI position ion) m/z: (M+H)+: 610.2 (calculated: 609.35)

Compound 298:

The intermediate compound 53 and the 5,6,7,8-tetrahydroimidazo[1,5-a]pyrazine (9 eq, 1.717 mmol, 283 mg) was used. The residue was purified by prep-HPLC (column: Phenomenex Gemini-NX C18 75*30 mm*3 um; mobile phase: [water(0.225% FA)-ACN];B %: 12%-42%, 7 min).

LCMS (ESI position ion) m/z: (M+H)+: 610.3 (calculated: 609.35)

Compound 299:

The intermediate compound 53 (50 mg, 95.60 umol, 1 eq) and the intermediate compound 76 (94.94 mg, 764.80 umol, 50.90 uL, 8 eq) was used. The residue was purified purified by prep-HPLC (column: Phenomenex luna C18 150*25 mm*10 um; mobile phase: [water(0.225% FA)-ACN];B %: 16%-46%, 10 min) followed by prep-HPLC (column: Waters Xbridge 150*25 mm*5 um; mobile phase: [water(10 mM NH4HCO3)-ACN];B %: 25%-55%, 10 min) to give the compound 299 (9.4 mg, 16% yield) as a yellow gum.

LCMS (ESI position ion) m/z: (M+H)+: 611.4 (calculated: 610.34)

Compound 302:

The intermediate compound 52 (50 mg, 95.60 umol, 1 eq) and the intermediate compound 17 (244.07 mg, 955.97 umol, 10 eq) was used. The residue was purified by prep-HPLC (column: Phenomenex Gemini-NX C18 75*30 mm*3 um; mobile phase: [water (0.225% FA)-ACN]; B %: 30%-60%, 7 min) to give the compound 302 (33.9 mg, 46% yield) as a yellow gum.

LCMS (ESI position ion) m/z: (M+H)+: 742.5 (calculated: 741.42)

Compound 303:

The intermediate compound 53 (50 mg, 95.60 umol, 1 eq) and the intermediate compound 18 (244.07 mg, 955.97 umol, 10 eq) was used. The residue was purified by prep-HPLC (column: Phenomenex Gemini-NX C18 75*30 mm*3 um; mobile phase: [water(0.225% FA)-ACN];B %: 30%-60%, 7 min) to give the compound 303 (32.4 mg, 44% yield) as a yellow gum.

LCMS (ESI position ion) m/z: (M+H)+: 742.4 (calculated: 741.42)

Compound 327:

The intermediate compound 52 (80 mg, 152.96 umol, 1 eq) and 1,1′-azanediylbis(propan-2-ol) (162.98 mg, 1.22 mmol, 162.98 uL, 8 eq) was used. The residue was purified two times by prep-HPLC (column: Phenomenex Gemini-NX C18 75*30 mm*3 um; mobile phase: [water(0.225% FA)-ACN];B %: 15%-45%, 7 min), followed by prep-HPLC (column: Waters Xbridge 150*25 mm*5 um; mobile phase: [water(10 mM NH4HCO3)-ACN];B %: 38%-68%, 10 min) to give the compound 327 (50.4 mg, 53% yield) as a yellow gum.

LCMS (ESI position ion) m/z: (M+H)+: 620.5 (calculated: 619.38)

General Procedure VI

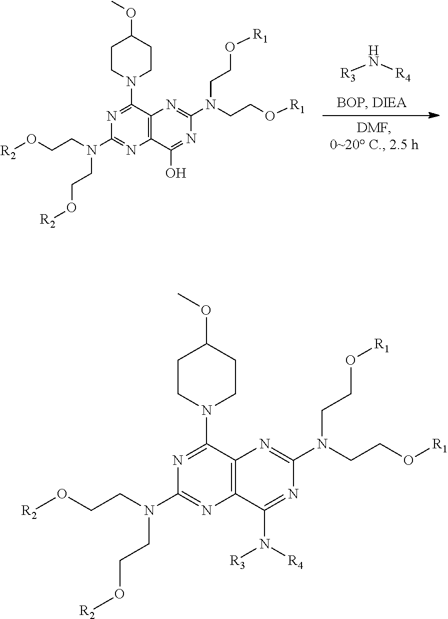

To a solution of either intermediate compound 87 or 88 (1 eq) in DMF (1 mL) was added DIPEA (1.5 eq) and BOP (1.3 eq) at 0° C., the mixture was stirred for 0.5 h. Then amine was added to the mixture. The mixture was stirred at 20° C. for 2 h. The reaction mixture was filtered and concentrated under vacuum.

Compound 307:

The intermediate compound 88 (80 mg, 161.43 umol, 1 eq) and 1-methylpiperazine-2,6-dione (62.05 mg, 484.29 umol, 3 eq) was used. The residue was purified by prep-HPLC (column: Phenomenex Gemini-NX C18 75*30 mm*3 um; mobile phase: [water(0.225% FA)-ACN];B %: 15%-45%, 7 min), followed by prep-HPLC (column: Phenomenex luna C18 150×25 mm×10 um; mobile phase: [water (0.225% FA)-ACN];B %: 14%-44%, 10 min) to give the compound 307 (33 mg, 33% yield) as a yellow gum.

LCMS (ESI position ion) m/z: (M+H)+: 606.4 (calculated: 605.33)

Compound 308:

The intermediate compound 87 (50 mg, 100.89 umol, 1 eq) and 1-methylpiperazine-2,6-dione (38.78 mg, 302.68 umol, 3 eq) was used. The residue was purified by prep-HPLC (column: Phenomenex luna C18 150*25 mm*10 um; mobile phase: [water(0.225% FA)-ACN];B %: 15%-45%, 10 min) to give the compound 308 (15 mg, 24% yield) as a yellow gum.

LCMS (ESI position ion) m/z: (M+H)+: 606.4 (calculated: 605.33)

Compound 309:

The intermediate compound 88 (60 mg, 121.07 umol, 1 eq) and intermediate compound 89 (48.89 mg, 205.82 umol, 1.70 eq) was used. The residue was purified by prep-HPLC (column: Phenomenex Gemini-NX C18 75*30 mm*3 um; mobile phase: [water(10 mM NH₄HCO₃)-ACN];B %: 24%-54%, 8 min) to give the compound 309 (44.2 mg, 60% yield) as a yellow gum.

LCMS (ESI position ion) m/z: (M+H)+: 606.5 (calculated: 605.36)

Compound 310:

The intermediate compound 87 (60 mg, 121.07 umol, 1 eq) and intermediate compound 89 (48.89 mg, 205.82 umol, 1.70 eq) was used. The residue was purified by prep-HPLC (column: Waters Xbridge 150*25 mm*5 um; mobile phase: [water(10 mM NH4HCO3)-ACN];B %: 33%-63%, 10 min) to give the compound 310 (33.7 mg, 46% yield) as a yellow gum.

LCMS (ESI position ion) m/z: (M+H)+: 606.5 (calculated: 605.36)

Compound 311:

The intermediate compound 88 (60 mg, 121.07 umol, 1 eq) and intermediate compound 90 (51.65 mg, 205.30 umol, 1.70 eq) was used. The residue was purified by prep-HPLC (column: Shim-pack C18 150*25*10 um; mobile phase: [water(0.225% FA)-ACN];B %: 15%-48%, 11 min) to give the compound 311 (45 mg, 57% yield) as a yellow gum.

LCMS (ESI position ion) m/z: (M+H)+: 620.6 (calculated: 619.38)

Compound 312:

The intermediate compound 87 (60 mg, 121.07 umol, 1 eq) and intermediate compound 90 (51.78 mg, 205.82 umol, 1.70 eq) was used. The residue was purified by prep-HPLC (column: Phenomenex Synergi C18 150*25 mm*10 um; mobile phase: [water(0.225% FA)-ACN];B %: 13%-46%, 11 min) to give the compound 312 (43.8 mg, 58% yield) as a yellow gum.

LCMS (ESI position ion) m/z: (M+H)+: 620.4 (calculated: 619.38)

Compound 313:

The intermediate compound 88 (60 mg, 121.07 umol, 1 eq) and hexahydropyrrolo[1,2-a]pyrazin-4(1H)-one (53.46 mg, 302.67 umol, 3 eq) was used. The residue was by prep-HPLC (column: Shim-pack C18 150×25×10 um; mobile phase: [water (0.225% FA)-ACN];B %: 12%-45%, 11 min) to give the compound 313 (31 mg, 48% yield) as a yellow gum.

LCMS (ESI position ion) m/z: (M+H)+: 618.4 (calculated: 617.36)

Compound 314:

The intermediate compound 87 (50 mg, 100.89 umol, 1 eq) and hexahydropyrrolo[1,2-a]pyrazin-4(1H)-one (42.43 mg, 240.20 umol, 2.38 eq) was used. The residue was purified by prep-HPLC (column: Phenomenex Gemini-NX C18 75*30 mm*3 um; mobile phase: [water(0.225% FA)-ACN];B %: 15%-45%, 7 min) to give the compound 314 (29.7 mg, 48% yield) as a yellow gum.

LCMS (ESI position ion) m/z: (M+H)+: 618.6 (calculated: 617.36)

Compound 315:

The intermediate compound 88 (40 mg, 80.71 umol, 1 eq) and intermediate compound 77 (60.00 mg, 428.01 umol, 5.30 eq) was used. The residue was by prep-HPLC (column: Phenomenex Gemini-NX C18 75*30 mm*3 um; mobile phase: [water(10 mM NH₄HCO₃)-ACN];B %: 24%-54%, 8 min) to give the compound 315 (26.7 mg, 53% yield) as a yellow gum.

LCMS (ESI position ion) m/z: (M+H)+: 618.3 (calculated: 617.36)

Compound 316:

The intermediate compound 87 (40 mg, 80.71 umol, 1 eq) and intermediate compound 77 (60 mg, 428.01 umol, 5.30 eq) was used. The residue was purified purified by prep-HPLC (column: Waters Xbridge 150*25 mm*5 um; mobile phase: [water(10 mM NH4HCO3)-ACN];B %: 34%-64%, 10 min) to give the compound 316 (26.5 mg, 50% yield) as a yellow gum.

LCMS (ESI position ion) m/z: (M+H)+: 618.3 (calculated: 617.36)

Compound 321:

The intermediate compound 91 (100 mg, 213.90 umol, 1 eq) and 1-methylpiperazin-2-one (60 mg, 428.01 umol, 5.30 eq) was used. The residue was purified purified by prep-HPLC (column: Shim-pack C18 150×25×10 um; mobile phase: [water (0.225% FA)-ACN]; B %: 9%-31%, 11 min) to give the compound 321 (37.0 mg, 31% yield,) as a yellow solid.

LCMS (ESI position ion) m/z: (M+H)+: 564.4 (calculated: 563.32)

Compound 322:

The intermediate compound 88 (50 mg, 100.89 umol, 1 eq) and 2-methyl-4,5,6,7-tetrahydrooxazolo[4,5-c]pyridine (52.86 mg, 302.68 umol, 3 eq) was used. The residue by prep-HPLC (column: Phenomenex Gemini-NX C18 75*30 mm*3 um; mobile phase: [water(0.225% FA)-ACN];B %: 18%-48%, 7 min). followed by prep-HPLC (column: Waters Xbridge 150*25 mm*5 um; mobile phase: [water(10 mM NH4HCO3)-ACN];B %: 39%-69%, 9 min) to give the compound 322 (29.4 mg, 47% yield) as a yellow gum.

LCMS (ESI position ion) m/z: (M+H)+: 616.4 (calculated: 615.35)

Compound 323:

The intermediate compound 87 (50 mg, 100.89 umol, 1 eq) and 2-methyl-5,6,7,8-tetrahydro-[1,2,4]triazolo[1,5-a]pyrazine (41.82 mg, 302.68 umol, 3 eq) was used. The residue was purified purified by prep-HPLC (column: Phenomenex Gemini-NX C18 75*30 mm*3 um; mobile phase: [water(0.225% FA)-ACN];B %: 15%-45%, 7 min) to give the compound 323 (27.5 mg, 44% yield) as a yellow gum.

LCMS (ESI position ion) m/z: (M+H)+: 616.4 (calculated: 615.36)

Compound 324:

The intermediate compound 87 (50 mg, 100.89 umol, 1 eq) and (3-fluorophenyl)methanamine (37.88 mg, 302.68 umol, 3 eq) was used. The residue was purified purified by prep-HPLC (column: Phenomenex Gemini-NX C18 75*30 mm*3 um; mobile phase: [water(0.225% FA)-ACN];B %: 22%-52%, 7 min) to give the compound 324 (26.6 mg, 43% yield) as a yellow solid.

LCMS (ESI position ion) m/z: (M+H)+: 603.5 (calculated: 602.33)

Compound 325:

The intermediate compound 87 (50 mg, 100.89 umol, 1 eq) and 3-(aminomethyl)benzonitrile (40.00 mg, 302.68 umol, 3 eq) was used. The residue was purified purified by prep-HPLC (column: Phenomenex Gemini-NX C18 75*30 mm*3 um; mobile phase: [water(0.225% FA)-ACN];B %: 20%-50%, 7 min) to give the compound 325 (27.4 mg, 43% yield) as a yellow solid.

LCMS (ESI position ion) m/z: (M+H)+: 610.6 (calculated: 609.34)

Compound 326:

The intermediate compound 87 (50 mg, 100.89 umol, 1 eq) and 3-(trifluoromethyl)azetidin-3-ol (53.74 mg, 302.68 umol, 3 eq) was used. The residue was purified purified by prep-HPLC (column: Phenomenex Gemini-NX C18 75×30 mm×3 um; mobile phase: [water(0.225% FA)-ACN];B %: 18%-48%, 2 min) to give the compound 326 (27.7 mg, 44% yield) as a yellow gum.

LCMS (ESI position ion) m/z: (M+H)+: 619.4 (calculated: 618.31)

Additional Analytical Data:

| Compounds | NMR |
|---|---|
| Compound | 1H NMR (400 MHz, CHLOROFORM-d) δ ppm 4.67 (br s, 2 H), 4.58- |
| 72 | 4.52 (m, 4 H), 3.78-3.74 (m, 8 H), 3.58 (m, 2 H), 3.57-3.55 (m, 8 |
| | H), 3.52-3.50 (m, 3 H), 3.39-3.38 (m, 3 H), 3.36-3.34 (m, 12 H), |
| | 3.02 (s, 3 H), 2.01-1.98 (m, 2 H), 1.68-1.64 (m, 2 H) |
| Compound | 1H NMR (400 MHz, CHLOROFORM-d) δ ppm 1.73-1.62 (m, 2 H) |
| 92 | 2.02-1.95 (m, 2 H) 2.92-2.82 (m, 1 H) 3.37 (m, 26 H) 3.63-3.49 |
| | (m, 16 H) 3.95-3.79 (m, 7 H) |
| Compound | 1H NMR (400 MHz, CHLOROFORM-d) δ ppm 1.79-1.59 (m, 2 H) |
| 93 | 2.17-1.88 (m, 4 H) 3.37 (m, 16 H) 4.35-3.40 (m, 20 H) 6.48-4.36 |
| | (m, 6 H) |
| Compound | 1H NMR (400 MHz, CHLOROFORM-d) δ ppm 1.78-1.32 (m, 6 H) |
| 104 | 2.84-1.82 (m, 6 H) 3.48-3.25 (m, 16 H) 4.25-3.49 (m, 16 H) |
| | 6.10-4.30 (m, 5 H) |
| Compound | 1H NMR (400 MHz, CHLOROFORM-d) δ ppm 2.11-1.93 (m, 4 H) |
| 105 | 3.35-3.32 (m, 5 H) 3.39-3.37 (m, 15 H) 3.63-3.55 (m, 4 H) 3.74- |
| | 3.63 (m, 12 H) 3.89-3.74 (m, 14 H) 4.05-3.91 (m, 4 H) 4.97-4.81 |
| | (m, 5 H) |
| Compound | 1H NMR (400 MHz, CHLOROFORM-d) δ ppm 1.61-1.56 (m, 3 H) |
| 106 | 1.64-1.62 (m, 3 H) 3.41-3.20 (m, 10 H) 3.56-3.52 (m, 6 H) 3.58- |
| | 3.57 (m, 12 H) 3.64-3.59 (m, 16 H) |
| Compound | 1H NMR (400 MHz, CHLOROFORM-d) δ ppm 1.78-1.46 (m, 6 H) |
| 109 | 2.10-1.89 (m, 2 H) 2.51-2.25 (m, 2 H) 3.45-3.26 (m, 15 H) 3.67- |
| | 3.46 (m, 10 H) 4.19-3.68 (m, 8 H) 4.79-4.25 (m, 3 H). |
| Compound | 1H NMR (400 MHz, CHLOROFORM-d) δ ppm 0.958 (d, J = 6.8, 2 |
| 127 | H) 1.649-1.567 (m, 2 H) 1.969-1.654 (m, 3 H) 3.585-3.360 (m, |
| | 17 H) 3.671 (s, 1 H) 3.842-3.772 (m, 9 H) 3.872-3.862 (m, 8 H) |
| | 4.857-4.826 (m, 2 H) 6.735-6.704 (m, 1 H). |
| Compound | 1H NMR (400 MHz, CHLOROFORM-d) δ ppm 2.12-1.87 (m, 5 H) |
| 136 | 2.63-2.36 (m, 2 H) 3.37-3.36 (m, 9 H) 3.42-3.37 (m, 18 H) 3.58- |
| | 3.42 (m, 16 H) |
| Compound | 1H NMR (400 MHz, CHLOROFORM-d) δ ppm 1.80-1.79 (m, 2 H) |
| 138 | 2.00-1.80 (m, 2 H) 3.33-3.29 (m, 3 H) 3.79-3.39 (m, 6 H) 3.81- |
| | 3.80 (m, 2 H) 3.82-3.81 (m, 3 H) 3.85-3.82 (m, 3 H) 3.86-3.86 |
| | (m, 19 H) 4.76-4.75 (m, 2 H) 7.14-7.14 (m, 1 H) 7.29-7.28 (m, 1 |
| | H) 7.36-7.35 (m, 1 H) |
| Compound | 1H NMR (400 MHz, CHLOROFORM-d) δ ppm 1.79-1.78 (m, 2 H) |
| 141 | 2.01-2.00 (m, 2 H) 3.39-3.27 (m, 3 H) 3.78-3.44 (m, 6 H) 3.81- |
| | 3.80 (m, 2 H) 3.84-3.82 (m, 3 H) 3.85-3.84 (m, 6 H) 3.90-3.85 |
| | (m, 16 H) 4.79-4.78 (m, 2 H) 7.28-7.27 (m, 4 H) |
| Compound | 1H NMR (400 MHz, CHLOROFORM-d) δ ppm 1.99-1.97 (m, 2 H) |
| 148 | 2.01-2.00 (m, 2 H) 3.36-3.35 (m, 3 H) 3.94-3.38 (m, 34 H) 4.88- |
| | 4.77 (m, 4 H) 7.62-7.53 (m, 1 H) 7.81-7.64 (m, 1 H) 7.83-7.81 |
| | (m, 1 H) 7.90-7.83 (m, 1 H) |
| Compound | 1H NMR (400 MHz, CHLOROFORM-d) δ ppm 1.79-1.64 (m, 2 H) |
| 150 | 2.03-1.93 (m, 2 H) 3.29 (s, 6 H) 3.36-3.33 (m, 6 H) 3.40-3.37 (m, |
| | 3 H) 3.55-3.40 (m, 5 H) 3.61-3.56 (m, 4 H) 3.72-3.63 (m, 5 H) |
| | 3.85-3.73 (m, 5 H) 4.68-4.63 (m, 4 H) 7.09-7.08 (m, 1 H) 7.46- |
| | 7.40 (m, 1 H) 7.54-7.47 (m, 1 H) 7.64-7.58 (m, 1 H) |
| Compound | 1H NMR (400 MHz, CHLOROFORM-d) δ ppm 1.68-1.67 (m, 2 H) |
| 154 | 2.00-1.97 (m, 2 H) 3.39-3.36 (m, 6 H) 3.62-3.50 (m, 6 H) 3.63- |
| | 3.62 (m, 3 H) 3.74-3.73 (m, 5 H) 3.82-3.74 (m, 4 H) 3.83-3.82 |
| | (m, 2 H) 3.85-3.83 (m, 4 H) 4.86-4.85 (m, 4 H) 4.75-4.73 (m, 2 |
| | H) 5.43-5.42 (m, 2 H) 7.40-7.30 (m, 1 H) 7.42-7.40 (m, 1 H) |
| | 7.58-7.57 (m, 1 H) |
| Compound | 1H NMR (400 MHz, CHLOROFORM-d) δ ppm 1.74-1.63 (m, 2 H) |
| 156 | 2.04-1.95 (m, 2 H) 3.41-3.30 (m, 15 H) 3.54-3.44 (m, 5 H) 3.65- |
| | 3.56 (m, 5 H) 3.78-3.66 (m, 5 H) 3.89-3.79 (m, 4 H) 4.90-4.58 |
| | (m, 2 H) 5.50 (s, 2 H) 7.50-7.44 (m, 1 H) 7.59 (m, 1 H) 7.72-7.68 |
| | (m, 1 H) 7.77 (s, 1 H) |
| Compound | 1H NMR (400 MHz, CHLOROFORM-d) δ ppm 1.68-1.67 (m, 2 H) |
| 159 | 1.99-1.98 (m, 2 H) 2.65 (s, 3 H) 3.34-3.31 (m, 6 H) 3.39-3.34 (m, |
| | 6 H) 3.51-3.39 (m, 3 H) 3.62-3.51 (m, 5 H) 3.63-3.62 (m, 4 H) |
| | 3.74-3.63 (m, 2 H) 3.75-3.74 (m, 4 H) 3.83-3.77 (m, 4 H) 4.73- |
| | 4.72 (m, 2 H) 5.55-5.54 (m, 2 H) 7.48-7.46 (m, 1 H) 7.62-7.48 |
| | (m, 1 H) 7.64-7.62 (m, 1 H) 8.15-8.00 (m, 1 H) |
| Compound | 1H NMR (400 MHz, CHLOROFORM-d) δ ppm 1.67-1.66 (m, 2 H) |
| 161 | 1.98-1.97 (m, 2 H) 2.00-1.98 (m, 3 H) 3.38-3.34 (m, 12 H) 3.46- |
| | 3.45 (m, 3 H) 3.56-3.46 (m, 1 H) 3.59-3.58 (m, 8 H) 3.76-3.59 |
| | (m, 2 H) 3.79-3.76 (m, 8 H) 4.62-4.60 (m, 2 H) 4.83 (s, 2 H) |
| | 5.88-5.87 (m, 1 H) 6.10-6.09 (m, 1 H) 6.84-6.81 (m, 1 H) |
| Compound | 1H NMR (400 MHz, CHLOROFORM-d) δ ppm 1.72-1.66 (m, 2 H) |
| 169 | 2.06-1.93 (m, 2 H) 3.25 (s, 8 H) 3.37-3.27 (m, 8 H) 3.44-3.37 (m, |
| | 7 H) 3.59-3.44 (m, 8 H) 3.69-3.60 (m, 4 H) 3.79-3.72 (m, 4 H) |
| | 3.83-3.80 (m, 3 H) 3.86 (s, 3 H) 4.82-4.59 (m, 2 H) 5.37 (s, 2 H) |
| | 6.86-6.76 (m, 3 H) |
| Compound | 1H NMR (400 MHz, CHLOROFORM-d) δ ppm 1.77-1.64 (m, 6 H) |
| 181 | 2.04-1.92 (m, 2 H) 3.34-3.32 (m, 3 H) 3.35-3.34 (m, 12 H) 3.39- |
| | 3.37 (m, 3 H) 3.43-3.39 (m, 2 H) 3.54-3.43 (m, 3 H) 3.66-3.65 |
| | (m, 10 H) 3.82-3.71 (m, 8 H) 5.01-4.60 (m, 2 H) 6.69-6.58 (m, 1 H) |
| Compound | 1H NMR (400 MHz, CHLOROFORM-d) δ ppm 1.74-1.63 (m, 2 H) |
| 196 | 2.05-1.94 (m, 2 H) 3.35 (s, 12 H) 3.39 (s, 3 H) 3.47-3.46 (m, 1 H) |
| | 3.64-3.51 (m, 14 H) 3.84-3.70 (m, 11 H) 4.34-4.15 (m, 4 H) |
| | 4.77-4.57 (m, 2 H) 7.73-7.69 (m, 1 H) |
| Compound | 1H NMR (400 MHz, CHLOROFORM-d) δ ppm 4.71 (br, 2 H), |
| 199 | 4.71-4.69, (m, 4 H), 3.78-3.76 (m, 8 H), 3.63-3.60(m, 10 H), |
| | 3.53-3.48 (m, 11 H), 3.40 (s, 3 H) 3.03 (s, 3 H), 2.02-1.99 (m, 2 H), |
| | 1.69-1.57 (m, 2 H), 1.22-1.18 (m, 12 H). |
| Compound | 1H NMR (400 MHz, CHLOROFORM-d) δ ppm 1.20 (s, 6 H) 1.67- |
| 201 | 1.66 (m, 4 H) 1.87-1.86 (m, 4 H) 3.23 (s, 6 H) 3.35 (s, 14 H) 4.31- |
| | 3.45 (m, 20 H) 5.09-4.34 (m, 2 H) 6.24-5.56 (m, 1 H) |
| Compound | 1H NMR (400 MHz, CHLOROFORM-d) δ ppm 7.95 (br s, 1 H), |
| 204 | 7.82 (br d, J = 7.28 Hz, 1H), 7.55 (br t, J = 7.28 Hz, 1H), 7.42-7.50 |
| | (m, 1H), 5.29 (s, 2H), 4.51-4.81 (m, 4H), 3.71-3.94 (m, 4H), 3.45- |
| | 3.68 (m, 14H), 3.38 (br s, 12H), 3.02 (s, 3H). |
| Compound | 1H NMR (400 MHz, CHLOROFORM-d) δ ppm 1.98-2.15 (m, 4 H) |
| 206 | 3.02-3.01 (m, 3 H) 3.35-3.34 (m, 12 H) 3.63-3.46 (m, 10 H) 3.85- |
| | 3.68 (m, 8 H) 4.40-4.13 (m, 4 H) 4.73-4.42 (m, 4 H) |
| Compound | 1H NMR (400 MHz, CHLOROFORM-d) δ ppm 2.00 (br s, 2 H) |
| 207 | 3.02 (br s, 3 H) 3.32 (br d, J = 14.18 Hz, 12 H) 3.49 (br s, 6 H) 3.55- |
| | 3.64 (m, 4 H) 3.67-3.76 (m, 4 H) 3.77-3.88 (m, 4 H) 4.48-4.87 |
| | (m, 5 H) 6.89-6.97 (m, 1 H) 6.99-7.06 (m, 2 H) 7.06-7.13 (m, 1 H) |
| Compound | 1H NMR (400 MHz, CHLOROFORM-d) δ ppm 1.55-2.04 (m, 2 H) |
| 208 | 2.91-3.16 (m, 3 H) 3.36 (br d, J = 13.82 Hz, 12 H) 3.46-3.68 (m, 10 |
| | H) 3.70-3.95 (m, 8 H) 4.49-4.98 (m, 5 H) 6.98-7.13 (m, 1 H) 7.26 |
| | (br s, 2 H) 7.32-7.42 (m, 1 H) |
| Compound | 1H NMR (400 MHz, CHLOROFORM-d) δ ppm 3.02 (br s, 3 H) |
| 209 | 3.32 (br d, J = 4.89 Hz, 12 H) 3.44-3.62 (m, 10 H) 3.65-4.00 (m, 12 |
| | H) 4.69 (br d, J = 4.40 Hz, 5 H) 6.72-6.84 (m, 1 H) 6.90-7.04 (m, 2 |
| | H) 7.18-7.25 (m, 1 H) |
| Compound | 1H NMR (400 MHz, CHLOROFORM-d) δ ppm 2.90-3.09 (m, 3 H) |
| 211 | 3.33 (br d, J = 16.02 Hz, 12 H) 3.41-3.64 (m, 10 H) 3.64-3.98 (m, 8 |
| | H) 4.46-4.84 (m, 5 H) 7.01-7.22 (m, 2 H) 7.49-7.68 (m, 2 H) |
| Compound | 1H NMR (400 MHz, CHLOROFORM-d) δ ppm 3.03-3.02 (m, 6 |
| 212 | H) 3.32-3.31 (m, 12 H) 3.63-3.42 (m, 10 H) 3.90-3.64 (m, 8 H) |
| | 4.92-4.48 (m, 6 H) 7.18-7.03 (m, 1 H) 7.57-7.45 (m, 1 H) 7.70- |
| | 7.59 (m, 1 H) 7.86-7.76 (m, 1 H) 7.97-7.86 (m, 1 H) |
| Compound | 1H NMR (400 MHz, CHLOROFORM-d) δ ppm 0.98-0.97 (m, 3 H) |
| 214 | 1.74-1.65 (m, 2 H) 3.09-2.94 (m, 3 H) 3.36-3.35 (m, 12 H) 3.52- |
| | 3.42 (m, 4 H) 3.59-3.58 (m, 8 H) 3.79-3.78 (m, 8 H) 4.98-4.34 |
| | (m, 4 H) 6.76-6.51 (m, 1 H) |
| Compound | 1H NMR (400 MHz, CHLOROFORM-d) δ ppm 0.97-0.96 (m, 6 |
| 215 | H) 2.03-1.87 (m, 1 H) 3.09-2.96 (m, 3 H) 3.43-3.27 (m, 14 H) |
| | 3.48-3.47 (m, 2 H) 3.68-3.53 (m, 8 H) 3.89-3.70 (m, 8 H) 4.89- |
| | 4.43 (m, 4 H) 6.80-6.65 (m, 1 H) |
| Compound | 1H NMR (400 MHz, CHLOROFORM-d) δ ppm 9.53-9.29 (m, 1H), |
| 216 | 4.68-4.59 (m, 2H), 4.58-4.45 (m, 2H), 4.19-4.09 (m, 4H), 4.02- |
| | 3.85 (m, 15H), 3.70-3.56 (m, 10H), 3.43-3.31 (m, 12H), 3.09- |
| | 3.00 (m, 3H) |
| Compound | 1H NMR (400 MHz, CHLOROFORM-d) δ ppm 3.28 (br d, J = 7.06 |
| 217 | Hz, 3H), 3.34-3.37 (m, 12H), 3.56-3.64 (m, 8H), 3.70 (br d, J = |
| | 5.29 Hz, 2H), 3.74-3.82 (m, 8H), 3.87 (br s, 3H), 4.00-4.14 (m, |
| | 4H), 4.39 (br s, 4H), 4.53 (br s, 2H), 4.95 (br s, 1H), 7.63-7.94 (m, |
| | 1H), 7.63-7.94 (m, 1H). |
| Compound | 1H NMR (400 MHz, CHLOROFORM-d) δ ppm 0.90-1.04 (m, 3H), |
| 218 | 1.25 (t, J = 6.97 Hz, 4H), 1.62-1.76 (m, 2H), 3.37 (s, 10H), 3.41- |
| | 3.50 (m, 2H), 3.51-3.68 (m, 11H), 3.68-3.86 (m, 11H), 4.18-4.54 |
| | (m, 3H), 7.72 (s, 1H) |
| Compound | 1H NMR (400 MHz, CHLOROFORM-d) δ ppm 7.72 (s, 1H), 3.71- |
| 219 | 3.90 (m, 10H), 3.50-3.70 (m, 14H), 3.24-3.49 (m, 14H), 1.49- |
| | 1.54 (m, 1H), 0.93-1.06 (m, 6H). |
| Compound | 1H NMR (400 MHz, CHLOROFORM-d) δ ppm 7.73 (s, 1H), 6.86- |
| 220 | 7.01 (m, 1H), 6.84-7.17 (m, 3H), 3.12-4.03 (m, 41H). |
| Compound | 1H NMR (400 MHz, CHLOROFORM-d) δ ppm 0.77-0.94 (m, 1H), |
| 221 | 1.11-1.38 (m, 5H), 3.27-3.38 (m, 10H), 3.47-3.62 (m, 10H), |
| | 3.71-3.84 (m, 10H), 4.28-4.48 (m, 3H), 4.61-4.79 (m, 2H), 6.95-7.16 |
| | (m, 1H), 7.18-7.25 (m, 2H), 7.32 (s, 1H), 7.72 (s, 1H). |
| Compound | 1H NMR (400 MHz, CHLOROFORM-d) δ ppm 7.72 (s, 1H), 7.20- |
| 222 | 7.25 (m, 1H), 6.87-7.02 (m, 3H), 6.80 (dd, J = 8.27, 2.09 Hz, 1H), |
| | 4.71 (br d, J = 5.29 Hz, 2H), 4.31-4.46 (m, 3H), 3.74-3.82 (m, |
| | 14H), 3.50-3.63 (m, 13H), 3.33 (d, J = 11.47 Hz, 12H). |
| Compound | 1H NMR (400 MHz, CHLOROFORM-d) δ ppm 7.26 (br s, 2H), |
| 223 | 7.18 (br s, 1H), 7.12 (br s, 2H), 4.70-4.78 (m, 2H), 4.28-4.51 (m, |
| | 3H), 3.78 (br s, 12H), 3.60 (br s, 12H), 3.22-3.42 (m, 13H). |
| Compound | 1H NMR (400 MHz, CHLOROFORM-d) δ ppm 1.52-1.68 (m, 3H), |
| 224 | 3.26-3.40 (m, 13H), 3.42-3.89 (m, 24H), 4.21-4.55 (m, 4H), |
| | 4.66-4.86 (m, 2H), 6.97-7.17 (m, 1H), 7.37-7.48 (m, 1H), 7.51-7.67 |
| | (m, 3H), 7.69-7.78 (m, 1H). |
| Compound | 1H NMR (400 MHz, CHLOROFORM-d) δ ppm 8.05 (s, 1H), 7.68- |
| 225 | 7.78 (m, 2H), 7.25-7.36 (m, 1H), 4.70 (s, 2H), 4.40 (br s, 3H), 3.84 |
| | (br t, J = 5.87 Hz, 4H), 3.77 (s, 3H), 3.66 (dt, J = 18.68, 5.95 Hz, |
| | 8H), 3.43-3.51 (m, 9H), 3.34-3.40 (m, 12H). |
| Compound | 1H NMR (400 MHz, CHLOROFORM-d) δ ppm 7.92 (s, 1H), 7.84 |
| 226 | (br d, J = 7.83 Hz, 1H), 7.72 (s, 1H), 7.65 (br d, J = 7.70 Hz, 1H), |
| | 7.49-7.57 (m, 1H), 7.03-7.16 (m, 1H), 4.68-4.92 (m, 2H), 4.26- |
| | 4.53 (m, 3H), 3.65-3.76 (m, 1H), 3.41-3.94 (m, 23H), 3.24-3.40 |
| | (m, 12H), 3.06 (s, 3H). |
| Compound | 1H NMR (400 MHz, CHLOROFORM-d) δ ppm 7.74 (s, 1H), 7.01- |
| 227 | 7.20 (m, 4H), 4.61-4.75 (m, 2H), 4.26-4.51 (m, 3H), 3.69-3.87 |
| | (m, 11H), 3.43-3.68 (m, 13H), 3.35 (br d, J = 18.34 Hz, 12H). |
| Compound | 1H NMR (400 MHz, CHLOROFORM-d) δ ppm 7.95 (s, 1H), 7.82 |
| 228 | (br d, J = 7.82 Hz, 1H), 7.72 (s, 1H), 7.52-7.58 (m, 1H), 7.41-7.48 |
| | (m, 1H), 5.34 (s, 2H), 4.59-4.82 (m, 2H), 4.22-4.50 (m, 3H), 3.73- |
| | 3.90 (m, 6H), 3.45-3.69 (m, 16H), 3.38 (br d, J = 5.75 Hz, 10H). |
| Compound | 1H NMR (400 MHz, CHLOROFORM-d) δ ppm 7.75-7.82 (m, 2H), |
| 229 | 7.72 (s, 1H), 7.52 (br d, J = 7.70 Hz, 1H), 7.38-7.44 (m, 1H), 7.14 |
| | (br s, 1H), 6.52-6.69 (m, 1H), 5.40-5.61 (m, 1H), 4.66-4.80 (m, |
| | 2H), 4.13-4.59 (m, 4H), 3.74-3.88 (m, 7H), 3.70 (br t, J = 6.05 Hz, |
| | 4H), 3.49-3.64 (m, 12H), 3.39-3.39 (m, 1H), 3.34 (d, J = 12.96 Hz, |
| | 11H). |
| Compound | 1H NMR (400 MHz, CHLOROFORM-d) δ ppm 7.28-7.31 (m, 1H), |
| 233 | 6.88-7.10 (m, 3H), 5.47 (br s, 2H), 4.42-4.71 (m, 4H), 3.77 (br t, |
| | J = 5.87 Hz, 4H), 3.64 (br s, 3H), 3.50-3.58 (m, 6H), 3.32-3.47 (m, |
| | 11H), 3.26 (br s, 8H), 3.03 (s, 3H). |
| Compound | 1H NMR (400 MHz, CHLOROFORM-d) δ ppm 7.29-7.37 (m, 2H), |
| 234 | 7.24 (s, 1H), 7.15 (br d, J = 6.84 Hz, 1H), 5.39-5.50 (m, 1H), 4.50- |
| | 4.65 (m, 3H), 3.76 (br d, J = 3.75 Hz, 4H), 3.61-3.69 (m, 6H), 3.51- |
| | 3.58 (m, 6H), 3.32-3.40 (m, 12H), 3.26 (br s, 5H), 3.19 (s, 2H), 3.03 |
| | (s, 3H). |
| Compound | 1H NMR (400 MHz, CHLOROFORM-d) δ ppm 7.23 (t, J = 7.83 Hz, |
| 235 | 1H), 6.75-6.88 (m, 3H), 5.31-5.58 (m, 2H), 4.43-4.70 (m, 4H), |
| | 3.73-3.82 (m, 7H), 3.65 (br s, 4H), 3.50-3.60 (m, 7H), 3.39 (br d, |
| | J = 9.70 Hz, 4H), 3.34 (s, 7H), 3.22-3.30 (m, 6H), 3.03 (s, 3H). |
| Compound | 1H NMR (400 MHz, CHLOROFORM-d) δ ppm 7.43 (t, J = 8.0 Hz, |
| 236 | 1H), 7.28 (d, J = 7.6 Hz, 1H), 7.20-7.13 (m, 2H), 5.53 (br s, 2H), |
| | 4.67-4.56 (m, 4H), 3.76 (t, J = 5.6 Hz, 4H), 3.65-3.44 (m, 12H), |
| | 3.42-3.33 (m, 12H), 3.25 (br s, 5H), 3.02 (s, 3H) |
| Compound | 1H NMR (400 MHz, CHLOROFORM-d) δ ppm 7.56 (br d, J = 4.41 |
| 237 | Hz, 1H), 7.53 (br d, J = 4.63 Hz, 1H), 7.51 (d, J = 2.21 Hz, 1H), 7.44 |
| | (br d, J = 7.50 Hz, 1H), 5.48 (br d, J = 2.43 Hz, 1H), 4.49-4.66 (m, |
| | 3H), 3.75 (br s, 5H), 3.49-3.63 (m, 11H), 3.31-3.39 (m, 12H), 3.25 |
| | (br s, 7H), 3.02 (s, 3H). |
| Compound | 1H NMR (400 MHz, CHLOROFORM-d) δ ppm 7.56-7.52 (m, 2H), |
| 238 | 7.20-7.16 (m, 1H), 5.44 (s, 2H), 4.60-4.55 (m, 4H), 3.78-3.74 (m, |
| | 4H), 3.57-3.51 (m, 9H), 3.45-3.35 (m, 11H), 3.27-3.24 (m, 9H), |
| | 3.03 (s, 3H) |
| Compound | 1H NMR (400 MHz, CHLOROFORM-d) δ ppm 3.01-3.05 (m, 1H), |
| 240 | 3.03 (s, 2H), 3.14-3.86 (m, 35H), 4.54 (br s, 3H), 5.49 (br s, 2H), |
| | 6.35 (br s, 1H), 7.43 (br s, 2H), 7.68-7.81 (m, 2H). |
| Compound | 1H NMR (400 MHz, CHLOROFORM-d) δ ppm 8.91 (br s, 1H), |
| 241 | 7.32-7.43 (m, 1H), 7.00-7.08 (m, 2H), 6.94-6.99 (m, 1H), 5.45-5.65 |
| | (m, 2H), 3.98 (br s, 9H), 3.83-3.93 (m, 5H), 3.60-3.75 (m, 8H), 3.35- |
| | 3.53 (m, 14H), 3.27 (br s, 6H). |
| Compound | 1H NMR (400 MHz, CHLOROFORM-d) δ ppm 9.17 (br s, 1H), |
| 242 | 7.32 (br s, 2H), 7.21-7.26 (m, 2H), 5.43-5.61 (m, 2H), 3.93-4.04 (m, |
| | 9H), 3.87 (br s, 5H), 3.61-3.75 (m, 8H), 3.35-3.51 (m, 14H), 3.28 (br |
| | s, 6H). |
| Compound | 1H NMR (400 MHz, CHLOROFORM-d) δ ppm 7.72 (s, 1H), 7.22 |
| 243 | (br t, J = 7.83 Hz, 1H), 6.81-6.91 (m, 2H), 6.78 (br d, J = 8.31 Hz, |
| | 1H), 5.14-5.69 (m, 2H), 4.16-4.44 (m, 3H), 3.48-3.91 (m, 23H), |
| | 3.14-3.46 (m, 18H). |
| Compound | 1H NMR (400 MHz, CHLOROFORM-d) δ ppm 7.72 (s, 1H), 7.60 |
| 244 | (s, 1H), 7.55 (br t, J = 6.97 Hz, 2H), 7.40-7.47 (m, 1H), 5.49 (br s, |
| | 1H), 4.28 (br s, 2H), 3.74-3.80 (m, 7H), 3.57 (br d, J = 6.11 Hz, |
| | 12H), 3.18-3.46 (m, 19H). |
| Compound | 1H NMR (400 MHz, CHLOROFORM-d) δ ppm 7.22 (s, 1H), 7.58- |
| 245 | 7.53 (m, 2H), 7.20-7.15 (m, 1H), 5.44 (s, 2H), 4.28-4.25 (m, 4H), |
| | 3.78-3.75 (m, 8H), 3.57-3.45 (m, 10H), 3.37-3.34 (m, 11H), 3.27- |
| | 3.23 (m, 9H) |
| Compound | 1H NMR (400 MHz, CHLOROFORM-d) δ ppm 7.89 (s, 1H), 7.84 |
| 246 | (d, J = 8.0 Hz, 1H), 7.72 (s, 1H), 7.60 (d, J = 8.0 Hz, 1H), 7.53 (t, J = |
| | 7.6 Hz, 1H), 5.53 (br s, 2H), 4.27 (br s, 4H), 3.88-3.69 (m, 8H), 3.63- |
| | 3.50 (m, 12H), 3.39-3.21 (m, 18H), 3.06 (s, 3H) |
| Compound | 1H NMR (400 MHz, CHLOROFORM-d) δ ppm 7.72 (s, 1H), 7.15- |
| 247 | 7.09 (m, 2H), 7.00-6.99 (m, 1H), 5.42-5.39 (m, 2H), 4.31-4.22 |
| | (m, 4H), 3.78-3.76 (m, 7H), 3.70-3.50 (m, 10H), 3.35-3.31 (m, |
| | 12H), 3.27-3.23 (m, 9H) |
| Compound | 1H NMR (400 MHz, CHLOROFORM-d) δ ppm 7.93 (s, 1H), 7.82- |
| 248 | 7.80 (m, 1H), 7.72 (s, 1H), 7.52-7.46 (m, 2H), 5.38-5.36 (m, 2H), |
| | 5.13 (s, 2H), 4.28-4.26 (m, 4H), 3.78 (s, 3H), 3.72-3.55 (m, 15H), |
| | 3.43-3.36 (m, 14H), 3.29 (s, 6H) |
| Compound | 1H NMR (400 MHz, CHLOROFORM-d) δ ppm 3.02-3.05 (m, 3H) |
| 250 | 3.37 (d, J = 2.65 Hz, 12H) 3.52 (t, J = 5.29 Hz, 2 H) 3.55-3.63 (m, 8 |
| | H) 3.72-3.88 (m, 8 H) 4.20-4.29 (m, 2 H) 4.44-4.75 (m, 6 H) 5.25 |
| | (br s, 2 H) 6.89 (s, 1 H) 7.49 (s, 1 H) |
| Compound | 1H NMR (400 MHz, CHLOROFORM-d) δ ppm 3.04 (s, 3H), 3.37 |
| 251 | (d, J = 1.32 Hz, 11H), 3.50-3.54 (m, 2H), 3.55-3.63 (m, 8H), 3.70- |
| | 3.75 (m, 2H), 3.75-3.87 (m, 8H), 4.52-4.74 (m, 6H), 5.11-5.41 |
| | (m, 2H), 7.58 (s, 1H). |
| Compound | 1H NMR (400 MHz, CHLOROFORM-d) δ ppm 7.15 (s, 1H), 5.04- |
| 252 | 4.94 (m, 1H), 4.58-4.52 (m, 5H), 3.86 (s, 3H), 3.84-3.77 (m, 8H), |
| | 3.61-3.58 (m, 10H), 3.37-3.36 (m, 12H), 3.03 (s, 3H), 2.99-2.97 |
| | (m, 2H), 1.53-1.50 (m, 2H) |
| Compound | 1H NMR (400 MHz, CHLOROFORM-d) δ ppm 7.39 (s, 1H), 5.11- |
| 253 | 4.82 (m, 2H), 4.67-4.36 (m, 6H), 3.87-3.76 (m, 8H), 3.63-3.57 |
| | (m, 11H), 3.52 (t, J = 5.6 Hz, 2H), 3.37 (d, J = 1.6 Hz, 12H), 3.03 (s, |
| | 3H), 2.85 (t, J = 4.8 Hz, 2H) |
| Compound | 1H NMR (400 MHz, CHLOROFORM-d) δ ppm 8.23 (s, 1H), 5.14- |
| 254 | 5.13 (m, 1H), 4.61-4.41 (m, 5H), 3.83-3.78 (m, 9H), 3.61-3.56 |
| | (m, 11H), 3.37-3.36 (m, 12H), 3.10-3.07 (m, 2H), 3.03 (s, 3H) |
| Compound | 1H NMR (400 MHz, CHLOROFORM-d) δ ppm 7.31 (s, 1H), 5.07- |
| 255 | 5.02 (m, 1H), 4.59-4.40 (m, 5H), 3.84-3.79 (m, 9H), 3.77 (s, 3H), |
| | 3.61-3.57 (m, 11H), 3.37-3.36 (m, 12H), 3.03 (s, 3H), 2.91-2.88 |
| | (m, 2H) |
| Compound | 1H NMR (400 MHz, CHLOROFORM-d) δ ppm 4.82 (br s, 1H), |
| 256 | 4.67-4.38 (m, 5H), 3.85-3.74 (m, 8H), 3.62-3.54 (m, 8H), 3.50 (t, |
| | J = 4.8 Hz, 2H), 3.41-3.29 (m, 14H), 3.02 (s, 3H), 2.96-2.84 (m, |
| | 2H), 2.47-2.41 (m, 3H) |
| Compound | 1H NMR (400 MHz, CHLOROFORM-d) δ ppm 7.05 (s, 1H), 6.89 |
| 257 | (s, 1H), 5.14-5.13 (m, 2H), 4.73-4.57 (m, 6H), 4.22-4.21 (m, |
| | 2H), 3.82-3.78 (m, 8H), 3.60-3.52 (m, 10H), 3.37-3.36 (m, 12H), |
| | 3.03 (s, 3H) |
| Compound | 1H NMR (400 MHz, CHLOROFORM-d) δ ppm 6.65-6.58 (m, 1H), |
| 258 | 6.17 (t, J = 2.8 Hz, 1H), 5.94 (s, 1H), 5.25 (brs, 2H), 4.75-4.47 (m, |
| | 6H), 4.17 (t, J = 5.2 Hz, 2H), 3.85-3.76 (m, 8H), 3.62-3.56 (m, |
| | 8H), 3.52 (t, J = 5.2 Hz, 2H), 3.37 (d, J = 2.8 Hz, 12H), 3.03 (s, 3H) |
| Compound | 1H NMR (400 MHz, CHLOROFORM-d) δ ppm 7.39 (s, 1H), 5.40- |
| 259 | 5.23 (m, 1H), 4.63-4.43 (m, 6H), 3.85-3.80 (m, 8H), 3.63-3.53 |
| | (m, 10H), 3.39-3.38 (m, 12H), 3.04 (s, 3H), 2.88-2.87 (m, 2H), |
| | 1.64-1.58 (m, 2H) |
| Compound | 1H NMR (400 MHz, CHLOROFORM-d) δ ppm 7.50 (d, J = 1.7 Hz, |
| 260 | 1H), 6.09 (s, 1H), 5.40-5.15 (m, 2H), 4.81-4.46 (m, 6H), 4.38 (t, J = |
| | 5.2 Hz, 2H), 3.86-3.75 (m, 8H), 3.64-3.55 (m, 8H), 3.52 (t, J = |
| | 4.8 Hz, 2H), 3.37 (d, J = 3.6 Hz, 12H), 3.04 (s, 3H) |
| Compound | 1H NMR (400 MHz, CHLOROFORM-d) δ ppm 8.16 (s, 1H), 4.97 |
| 261 | (brs, 2H), 4.70-4.32 (m, 6H), 3.87-3.75 (m, 8H), 3.63-3.56 (m, |
| | 8H), 3.52 (t, J = 5.6 Hz, 2H), 3.37 (d, J = 3.2 Hz, 12H), 3.12-2.99 |
| | (m, 5H) |
| Compound | 1H NMR (400 MHz, CHLOROFORM-d) δ ppm 7.25 (br, 4H), 3.94- |
| 264 | 3.82 (m, 5H), 3.81(m, 3H), 3.69 (m, 7H), 3.63(m, 3H), 3.38(m, |
| | 6H), 3.36 (s, 9H), 3.33 (s, 6H), 2.99 (s, 2 H), 2.04 (m, 4H), 1.80 (m, |
| | 4H). |
| Compound | 1H NMR (400 MHz, CHLOROFORM-d) δ ppm 4.33 (br, 2H), 3.89 |
| 265 | (m, 2H), 3.84(m, 2H), 3.77 (m, 2H), 3.75(m, 6H), 3.67(m, 2H), 3.64 |
| | (s, 4H), 3.63 (s, 4H), 3.33 (s, 14H), 2.70 (m, 2H), 1.94 (m, 4H), 1.70 |
| | (m, 4H) |
| Compound | 1H NMR (400 MHz, CHLOROFORM-d) δ ppm 7.30 (s, 1H), 7.14 |
| 266 | (s, 1H), 4.93-5.32 (m, 2H), 4.46-4.69 (m, 4H), 4.25-4.46 (m, 2H), |
| | 3.74-3.90 (m, 11H), 3.55-3.64 (m, 8H), 3.52 (br t, J = 5.26 Hz, |
| | 2H), 3.29-3.42 (m, 12H), 3.03 (s, 3H), 2.76-2.88 (m, 2H) |
| Compound | 1H NMR (400 MHz, CHLOROFORM-d) δ ppm 8.62-8.59 (m, |
| 267 | 0.4H), 4.77-4.46 (m, 6H), 3.97-3.85 (m, 2H), 3.84-3.69 (m, 5H), |
| | 3.69-3.55 (m, 8H), 3.52-3.45 (m, 3H), 3.41-3.38 (s, 3H), 3.36 (s, |
| | 6H), 3.19-3.09 (m, 5H), 3.02 (s, 3H), 2.04-1.97 (m, 6H), 1.71- |
| | 1.62 (m, 2H), 1.20-1.13 (m, 3H) |
| Compound | 1H NMR (400 MHz, CHLOROFORM-d) δ ppm 6.49 (s, 2H), 4.85- |
| 268 | 4.79 (m, 2H), 4.68-4.38 (m, 6H), 3.86-3.75 (m, 16H), 3.63-3.56 |
| | (m, 7H), 3.48-3.47 (m, 1H), 3.39-3.32 (m, 14H), 3.03-2.98 (m, |
| | 3H), 2.02-1.83 (m, 2H), 1.31-1.25 (m, 2H) |
| Compound | 1H NMR (400 MHz, CHLOROFORM-d) δ ppm 6.51 (s, 2H), 4.80 |
| 269 | (s, 2H), 4.75-4.32 (m, 6H), 3.86-3.72 (m, 15H), 3.71-3.54 (m, |
| | 8H), 3.48 (td, J = 4.0, 7.8 Hz, 1H), 3.43-3.27 (m, 14H), 2.98 (s, |
| | 3H), 1.97 (br s, 2H), 1.72-1.67 (m, 2H) |
| Compound | 1H NMR (400 MHz, CHLOROFORM-d) δ ppm 4.77-4.45 (m, 4H), |
| 270 | 3.99-3.88 (m, 4H), 3.83-3.71 (m, 8H), 3.68-3.45 (m, 10H), 3.42- |
| | 3.31 (m, 10H), 3.08-2.99 (m, 3H), 2.07-1.97 (m, 2H), 1.73 (br s, |
| | 2H) |
| Compound | 1H NMR (400 MHz, CHLOROFORM-d) δ ppm 4.76 (br s, 2H), |
| 271 | 4.49 (br d, J = 4.6 Hz, 1H), 4.37 (br s, 1H), 4.14-3.87 (m, 5H), 3.77 |
| | (br d, J = 5.8 Hz, 9H), 3.56 (br t, J = 5.8 Hz, 8H), 3.43-3.38 (m, |
| | 4H), 3.35 (s, 8H), 3.02 (s, 3H), 2.00 (br s, 3H), 1.78 (br s, 1H) |
| Compound | 1H NMR (400 MHz, CHLOROFORM-d) δ ppm 8.59 (s, 1H), 4.74- |
| 272 | 4.48 (m, 5H), 4.01-3.87 (m, 2H), 3.81-3.71 (m, 4H), 3.71-3.53 |
| | (m, 8H), 3.53-3.45 (m, 3H), 3.44-3.27 (m, 10H), 3.12-2.93 (m, |
| | 9H), 2.04-1.84 (m, 6H), 1.72-1.55 (m, 4H), 1.17 (t, J = 7.0 Hz, 3H) |
| Compound | 1H NMR (400 MHz, CHLOROFORM-d) δ ppm 4.75-4.50 (m, 6H), |
| 273 | 3.82-3.64 (m, 10H), 3.61-3.55 (m, 6H), 3.51 (br t, J = 5.2 Hz, 3H), |
| | 3.40 (s, 3H), 3.39-3.29 (m, 9H), 3.04 (s, 3H), 2.40-2.29 (m, 2H), |
| | 2.06-1.91 (m, 4H), 1.75-1.62 (m, 2H) |
| Compound | 1H NMR (400 MHz, CHLOROFORM-d) δ ppm 5.27-4.95 (m, 2H), |
| 274 | 4.72-4.47 (m, 2H), 4.21-4.04 (m, 2H), 3.92-3.63 (m, 10H), 3.56 |
| | (br d, J = 5.1 Hz, 9H), 3.40 (s, 3H), 3.38-3.33 (m, 9H), 3.03 (s, 3H), |
| | 2.45-2.31 (m, 2H), 2.00 (br d, J = 1.1 Hz, 4H), 1.71 (br dd, J = 2.0, |
| | 8.4 Hz, 2H) |
| Compound | 1H NMR (400 MHz, CHLOROFORM-d) δ ppm 4.63-4.51 (m, 5H), |
| 275 | 3.87-3.81 (m, 2H), 3.80-3.68 (m, 10H), 3.66-3.55 (m, 7H), 3.53- |
| | 3.46 (m, 3H), 3.41-3.34 (m, 13H), 3.03 (s, 3H), 2.06-1.95 (m, 2H), |
| | 1.73-1.64 (m, 2H) |
| Compound | 1H NMR (400 MHz, CHLOROFORM-d) δ ppm 4.89-4.49 (m, 4H), |
| 276 | 4.44-4.18 (m, 2H), 3.88 (br s, 2H), 3.80-3.74 (m, 8H), 3.67 (br d, J = |
| | 5.1 Hz, 2H), 3.60-3.47 (m, 8H), 3.40 (s, 3H), 3.38-3.33 (m, |
| | 10H), 3.02 (s, 3H), 1.99 (br d, J = 3.0 Hz, 2H), 1.74-1.70 (m, 2H) |
| Compound | 1H NMR (400 MHz, CHLOROFORM-d) δ ppm 4.69-4.45 (m, 9H), |
| 277 | 3.86-3.81 (m, 3H), 3.80-3.71 (m, 12H), 3.63-3.55 (m, 12H), 3.53- |
| | 3.45 (m, 5H), 3.40-3.38 (m, 5H), 3.37-3.35 (m, 10H), 3.07-2.98 |
| | (m, 9H), 2.07-2.03 (m, 3H), 2.03-1.95 (m, 6H), 1.73-1.62 (m, |
| | 4H) |
| Compound | 1H NMR (400 MHz, CHLOROFORM-d) δ ppm 6.30 (br s, 1H), |
| 278 | 4.66 (br s, 3H), 4.44 (br s, 2H), 4.33 (s, 2H), 3.94 (t, J = 5.3 Hz, 2H), |
| | 3.83-3.63 (m, 6H), 3.61-3.44 (m, 10H), 3.40 (s, 3H), 3.35 (s, 6H), |
| | 3.02 (s, 3H), 2.06-1.96 (m, 2H), 1.74-1.63 (m, 2H) |
| Compound | 1H NMR (400 MHz, CHLOROFORM-d) δ ppm 4.62 (br s, 3H), |
| 279 | 4.30 (br s, 2H), 3.73 (br s, 8H), 3.62-3.37 (m, 9H), 3.36-3.08 (m, |
| | 14H), 2.95 (s, 3H), 2.72 (s, 3H), 1.94 (s, 2H), 1.62 (br s, 2H) |
| Compound | 1H NMR (400 MHz, METHANOL-d4) δ ppm (m, 6H), 3.84-3.48 |
| 280 | (m, 19H), 3.43-3.37 (m, 9H), 3.43-3.36 (m, 2H), 3.43-3.30 (m, |
| | 6H), 3.23-3.17 (m, 2H), 2.10-2.01 (m, 2H), 1.69-1.59 (m, 2H) |
| Compound | 1H NMR (400 MHz, CHLOROFORM-d) δ ppm 4.72 (br s, 4H), |
| 281 | 4.38 (br s, 2H), 3.83-3.76 (m, 4H), 3.75-3.63 (m, 8H), 3.60-3.48 |
| | (m, 9H), 3.40 (s, 3H), 3.35 (s, 6H), 3.03 (s, 3H), 2.15 (s, 3H), 2.06- |
| | 1.96 (m, 2H), 1.74-1.71 (m, 2H) |
| Compound | 1H NMR (400 MHz, CHLOROFORM-d) δ ppm 7.92 (s, 1H), 5.01- |
| 282 | 4.96 (m, 2H), 4.70-4.57 (m, 5H), 4.31-4.25 (m, 4H), 3.80-3.68 |
| | (m, 6H), 3.61-3.48 (m, 8H), 3.41 (s, 3H), 3.37 (s, 6H), 3.05 (s, 3H), |
| | 2.06-2.00 (m, 2H), 1.72-1.70 (m, 2H) |
| Compound | 1H NMR (400 MHz, CHLOROFORM-d) δ ppm 7.92 (s, 1H), 4.99 |
| 283 | (s, 2H), 4.68 (br s, 4H), 4.43 (br s, 2H), 4.28 (br dd, J = 3.8, 11.4 Hz, |
| | 4H), 3.79 (br t, J = 5.6 Hz, 6H), 3.64-3.49 (m, 7H), 3.41 (s, 3H), |
| | 3.35 (s, 6H), 3.04 (s, 3H), 2.01 (br s, 2H), 1.76-1.68 (m, 2H) |
| Compound | 1H NMR (400 MHz, CHLOROFORM-d) δ ppm 4.68 (br s, 3H), |
| 284 | 4.40 (br s, 2H), 3.79 (br t, J = 5.5 Hz, 4H), 3.76-3.62 (m, 10H), 3.62- |
| | 3.45 (m, 11H), 3.40 (s, 3H), 3.35 (s, 6H), 3.03 (s, 3H), 2.01 (br d, J = |
| | 6.6 Hz, 2H), 1.73-1.69 (m, 2H) |
| Compound | 1H NMR (400 MHz, CHLOROFORM-d) δ ppm 7.42 (s, 1H), 7.04 |
| 285 | (s, 1H), 6.94 (s, 1H), 4.75-4.49 (m, 6H), 4.22 (t, J = 6.2 Hz, 2H), |
| | 3.91 (br t, J = 6.2 Hz, 2H), 3.81-3.75 (m, 4H), 3.73-3.55 (m, 8H), |
| | 3.54-3.46 (m, 7H), 3.40-3.39 (m, 3H), 3.37 (s, 6H), 3.31 (s, 3H), |
| | 3.04 (s, 3H), 2.03-1.96 (m, 2H), 1.70-1.67 (m, 2H). |
| Compound | 1H NMR (400 MHz, CHLOROFORM-d) δ ppm 7.50-7.46 (m, 1H), |
| 286 | 7.10-7.05 (m, 1H), 6.99-6.95 (m, 1H), 4.84-4.56 (m, 4H), 4.45- |
| | 4.36 (m, 2H), 4.28-4.21 (m, 2H), 3.92 (br t, J = 5.6 Hz, 2H), 3.83- |
| | 3.78 (m, 4H), 3.77-3.64 (m, 3H), 3.60 (br t, J = 5.6 Hz, 4H), 3.56- |
| | 3.45 (m, 8H), 3.42 (s, 3H), 3.37 (s, 6H), 3.34 (s, 3H), 3.04 (s, 3H), |
| | 2.06-2.00 (m, 2H), 1.72 (br s, 2H). |
| Compound | 1H NMR (400 MHz, CHLOROFORM-d) δ ppm 7.65-7.55 (m, 1H), |
| 287 | 4.97 (s, 2H), 4.69 (br d, J = 1.4 Hz, 2H), 4.60 (br s, 2H), 4.50 (br t, J = |
| | 5.3 Hz, 2H), 4.24 (br t, J = 5.4 Hz, 2H), 3.82-3.75 (m, 6H), 3.60- |
| | 3.51 (m, 8H), 3.42 (s, 3H), 3.38-3.35 (m, 7H), 3.07-3.03 (m, 3H), |
| | 2.03 (ddd, J = 3.0, 6.3, 9.3 Hz, 2H), 1.70 (br s, 2H) |
| Compound | 1H NMR (400 MHz, CHLOROFORM-d) δ ppm 7.61 (s, 1H), 4.97 |
| 288 | (s, 2H), 4.86-4.56 (m, 4H), 4.51 (t, J = 5.3 Hz, 2H), 4.36 (br s, 2H), |
| | 4.24 (t, J = 5.3 Hz, 2H), 3.86-3.71 (m, 6H), 3.60-3.49 (m, 7H), |
| | 3.41 (s, 3H), 3.35 (s, 6H), 3.04 (s, 3H), 2.02 (br s, 2H), 1.77-1.67 |
| | (m, 2H) |
| Compound | 1H NMR (400 MHz, METHANOL-d4) δ ppm 3.83-3.73 (m, 6H), |
| 289 | 3.83-3.73 (m, 8H), 3.66-3.59 (m, 7H), 3.59-3.51 (m, 3H), 3.43- |
| | 3.41 (m, 3H), 3.38 (s, 9H), 3.06-3.01 (m, 2H), 3.01-2.95 (m, 3H), |
| | 2.26-2.14 (m, 2H), 2.10-1.98 (m, 2H), 1.85-1.75 (m, 2H), 1.60- |
| | 1.48 (m, 2H) |
| Compound | 1H NMR (400 MHz, CHLOROFORM-d) δ ppm 8.24 (br s, 1H), |
| 290 | 4.86-4.74 (m, 2H), 4.69-4.56 (m, 4H), 4.08-3.96 (m, 5H), 3.76 |
| | (br s, 4H), 3.60-3.56 (m, 5H), 3.55-3.49 (m, 4H), 3.39 (s, 3H), |
| | 3.36 (s, 6H), 3.05-3.02 (m, 3H), 2.66 (d, J = 9.3 Hz, 1H), 2.05- |
| | 1.99 (m, 2H), 1.67 (br d, J = 9.0 Hz, 2H) |
| Compound | 1H NMR (400 MHz, CHLOROFORM-d) δ ppm 8.21 (s, 1H), 5.17- |
| 291 | 4.54 (m, 6H), 4.44-3.96 (m, 6H), 3.77 (br s, 5H), 3.61-3.47 (m, |
| | 7H), 3.39 (s, 3H), 3.37-3.31 (m, 6H), 3.07-2.97 (m, 3H), 2.66 (d, J = |
| | 9.4 Hz, 1H), 2.00 (br s, 2H), 1.68 (br d, J = 9.2 Hz, 2H) |
| Compound | 1H NMR (400 MHz, CHLOROFORM-d) δ ppm 4.92-4.19 (m, 8H), |
| 292 | 3.87-3.63 (m, 6H), 3.62-3.45 (m, 7H), 3.38 (d, J = 19.8 Hz, 9H), |
| | 3.16-2.99 (m, 4H), 2.94-2.81 (m, 5H), 2.20 (br d, J = 11.3 Hz, 2H), |
| | 2.02 (br d, J = 13.3 Hz, 2H), 1.81 (br d, J = 10.4 Hz, 2H), 1.69 (br s, |
| | 2H) |
| Compound | 1H NMR (400 MHz, CHLOROFORM-d) δ ppm 7.85 (br d, J = 4.9 |
| 293 | Hz, 1H), 7.10-7.00 (m, 1H), 6.99-6.90 (m, 1H), 4.73-4.53 (m, |
| | 6H), 3.94-3.84 (m, 4H), 3.78 (br t, J = 6.1 Hz, 4H), 3.73-3.63 (m, |
| | 2H), 3.61-3.57 (m, 4H), 3.55-3.45 (m, 4H), 3.40 (s, 3H), 3.37 (s, |
| | 7H), 3.28 (br d, J = 1.2 Hz, 4H), 3.06-3.02 (m, 3H), 2.05-1.98 (m, |
| | 2H), 1.70 (br s, 2H) |
| Compound | 1H NMR (400 MHz, CHLOROFORM-d) δ ppm 7.82 (dd, J = 1.5, |
| 294 | 4.9 Hz, 1H), 7.00 (d, J = 7.6 Hz, 1H), 6.88 (dd, J = 4.8, 7.6 Hz, 1H), |
| | 4.67 (br s, 4H), 4.45 (br s, 2H), 3.94-3.76 (m, 10H), 3.68 (br s, 2H), |
| | 3.58 (t, J = 5.9 Hz, 4H), 3.55-3.47 (m, 3H), 3.40 (s, 3H), 3.35 (s, |
| | 6H), 3.23 (br s, 4H), 3.02 (s, 3H), 2.02 (br dd, J = 3.3, 9.3 Hz, 2H), |
| | 1.75-1.71 (m, 2H) |
| Compound | 1H NMR (400 MHz, CHLOROFORM-d) δ ppm 7.25-7.19 (m, 1H), |
| 295 | 7.03-6.97 (m, 1H), 5.16 (br s, 2H), 4.78-4.57 (m, 5H), 4.29-4.12 |
| | (m, 4H), 3.88-3.64 (m, 7H), 3.60-3.50 (m, 7H), 3.43-3.35 (m, |
| | 9H), 3.04 (s, 3H), 2.12-2.04 (m, 2H), 1.69 (br dd, J = 3.7, 8.4 Hz, |
| | 2H) |
| Compound | 1H NMR (400 MHz, CHLOROFORM-d) δ ppm 7.36 (s, 1H), 7.09 |
| 296 | (s, 1H), 5.30 (s, 2H), 4.57 (br s, 5H), 4.28 (br s, 4H), 3.80 (br s, 7H), |
| | 3.60-3.50 (m, 7H), 3.41 (s, 3H), 3.36-3.35 (m, 6H), 3.10 (s, 3H), |
| | 2.02 (br s, 2H), 1.71-1.68 (m, 2H) |
| Compound | 1H NMR (400 MHz, CHLOROFORM-d) δ ppm 7.86 (s, 1H), 7.04- |
| 297 | 6.94 (m, 1H), 4.91 (s, 2H), 4.72-4.65 (m, 2H), 4.64-4.57 (m, 3H), |
| | 4.23-4.14 (m, 4H), 3.79-3.75 (m, 4H), 3.67 (br d, J = 8.6 Hz, 2H), |
| | 3.60-3.56 (m, 5H), 3.55-3.50 (m, 3H), 3.41 (s, 3H), 3.36 (s, 6H), |
| | 3.05 (s, 3H), 2.00 (br d, J = 2.9 Hz, 2H), 1.73-1.67 (m, 2H) |
| Compound | 1H NMR (400 MHz, CHLOROFORM-d) δ ppm 7.53 (s, 1H), 6.93 |
| 298 | (s, 1H), 4.90 (s, 2H), 4.82-4.64 (m, 3H), 4.39 (br s, 2H), 4.14 (s, |
| | 4H), 3.83-3.65 (m, 6H), 3.61-3.45 (m, 8H), 3.41 (s, 3H), 3.35 (s, |
| | 6H), 3.04 (s, 3H), 2.03 (br dd, J = 2.9, 13.0 Hz, 2H), 1.71-1.67 (m, |
| | 2H) |
| Compound | 1H NMR (400 MHz, CHLOROFORM-d) δ ppm 8.15 (s, 1H), 5.08 |
| 299 | (s, 2H), 4.70 (br s, 3H), 4.40 (br s, 2H), 4.22-4.13 (m, 4H), 3.79 (br |
| | t, J = 5.9 Hz, 6H), 3.62-3.46 (m, 8H), 3.41 (s, 3H), 3.35 (s, 6H), |
| | 3.04 (s, 3H), 2.07-1.96 (m, 2H), 1.75-1.63 (m, 2H). |
| Compound | 1H NMR (400 MHz, CHLOROFORM-d) δ ppm 6.50 (s, 2H), 4.80 |
| 300 | (s, 2H), 4.76-4.59 (m, 2H), 4.58-4.30 (m, 4H), 3.85 (br d, J = 7.4 |
| | Hz, 2H), 3.83 (s, 3H), 3.80 (s, 6H), 3.77 (br d, J = 6.1 Hz, 2H), 3.71- |
| | 3.56 (m, 6H), 3.51-3.45 (m, 1H), 3.39 (s, 3H), 3.35 (s, 3H), 3.21- |
| | 3.07 (m, 6H), 2.97 (s, 3H), 2.82-2.68 (m, 2H), 2.08-1.92 (m, 6H), |
| | 1.72-1.60 (m, 2H), 1.17 (t, J = 6.9 Hz, 3H) |
| Compound | 1H NMR (400 MHz, CHLOROFORM-d) δ ppm 5.23-5.10 (m, 2H), |
| 301 | 4.79-4.46 (m, 6H), 4.33 (br t, J = 4.9 Hz, 2H), 3.81 (q, J = 6.0 Hz, |
| | 8H), 3.63-3.56 (m, 8H), 3.55-3.50 (m, 2H), 3.36 (s, 12H), 3.04 (s, |
| | 3H), 2.41 (s, 3H) |
| Compound | 1H NMR (400 MHz, CHLOROFORM-d) δ ppm 6.76 (d, J = 8.6 Hz, |
| 302 | 1H), 6.53 (d, J = 8.7 Hz, 1H), 4.84 (s, 2H), 4.62 (br s, 4H), 4.52- |
| | 4.35 (m, 2H), 3.89 (d, J = 2.2 Hz, 6H), 3.82 (s, 3H), 3.80-3.71 (m, |
| | 6H), 3.71-3.49 (m, 9H), 3.45 (br d, J = 2.4 Hz, 2H), 3.42-3.34 (m, |
| | 9H), 3.33 (s, 3H), 3.00 (s, 3H), 1.97-1.79 (m, 2H), 1.27 (br s, 2H) |
| Compound | 1H NMR (400 MHz, CHLOROFORM-d) δ ppm 6.77 (d, J = 8.6 Hz, |
| 303 | 1H), 6.56-6.56 (m, 1H), 6.55 (d, J = 8.7 Hz, 1H), 4.82 (br s, 2H), |
| | 4.78-4.58 (m, 2H), 4.58-4.30 (m, 4H), 3.90 (d, J = 4.0 Hz, 6H), |
| | 3.82 (s, 3H), 3.81-3.73 (m, 6H), 3.73-3.52 (m, 9H), 3.51-3.40 (m, |
| | 2H), 3.38 (s, 3H), 3.35 (s, 9H), 2.97 (br s, 3H), 2.06-1.92 (m, 2H), |
| | 1.27 (br s, 2H) |
| Compound | 1H NMR (400 MHz, CHLOROFORM-d) δ ppm 6.75 (d, J = 8.6 Hz, |
| 304 | 1H), 6.56 (d, J = 8.6 Hz, 1H), 4.81 (br s, 2H), 4.47 (br d, J = 4.4 Hz, |
| | 6H), 3.92-3.84 (m, 11H), 3.82 (s, 3H), 3.76 (br s, 6H), 3.64-3.53 |
| | (m, 4H), 3.46 (br s, 2H), 3.37 (s, 3H), 3.34 (s, 3H), 2.96 (br s, 3H), |
| | 1.97 (br s, 2H), 1.67 (br s, 2H) |
| Compound | 1H NMR (400 MHz, CHLOROFORM-d) δ ppm 6.74 (d, J = 8.8 Hz, |
| 305 | 1H), 6.55 (d, J = 8.8 Hz, 1H), 4.83 (br s, 4H), 4.65-4.43 (m, 2H), |
| | 4.23-4.05 (m, 2H), 3.89 (d, J = 2.4 Hz, 11H), 3.82 (s, 3H), 3.75 (br |
| | s, 6H), 3.68-3.48 (m, 4H), 3.44 (br d, J = 1.6 Hz, 2H), 3.38-3.31 |
| | (m, 6H), 2.98 (br s, 3H), 1.96-1.85 (m, 2H), 1.65-1.50 (m, 2H) |
| Compound | 1H NMR (400 MHz, CHLOROFORM-d) δ ppm 4.72 (br s, 2H), |
| 306 | 4.54 (br s, 2H), 4.41 (br s, 2H), 3.95-3.74 (m, 14H), 3.68 (td, J = |
| | 5.0, 10.1 Hz, 6H), 3.50 (br t, J = 5.4 Hz, 3H), 3.40 (s, 3H), 3.37 (d, |
| | J = 3.1 Hz, 6H), 3.03 (s, 3H), 1.98 (br d, J = 3.3 Hz, 2H), 1.70 (br dd, |
| | J = 4.0, 8.5 Hz, 2H) |
| Compound | 1H NMR (400 MHz, CHLOROFORM-d) δ ppm 5.09-4.90 (m, 3H), |
| 307 | 4.54-4.36 (m, 2H), 3.98-3.87 (m, 4H), 3.86-3.67 (m, 10H), 3.65- |
| | 3.47 (m, 6H), 3.39 (br d, J = 3.8 Hz, 9H), 3.15 (s, 3H), 2.04-1.96 |
| | (m, 2H), 1.73-1.69 (m, 2H) |
| Compound | 1H NMR (400 MHz, CHLOROFORM-d) δ ppm 4.92 (s, 4H), 4.59 |
| 308 | (s, 2H), 3.88 (t, J = 4.8 Hz, 4H), 3.79-3.77 (m, 9H), 3.56-3.54 (m, |
| | 5H), 3.39 (s, 3H), 3.35 (s, 6H), 3.17 (s, 3H), 2.02-1.98 (m, 2H), |
| | 1.73-1.67 (m, 2H) |
| Compound | 1H NMR (400 MHz, METHANOL-d4) δ ppm 5.17-5.06 (m, 1H), |
| 309 | 5.04-4.93 (m, 1H), 4.77-4.66 (m, 2H), 4.43-4.35 (m, 1H), 4.19- |
| | 4.12 (m, 1H), 3.83-3.66 (m, 14H), 3.62-3.50 (m, 6H), 3.39 (s, 3H), |
| | 3.36 (s, 6H), 3.03-2.99 (m, 3H), 2.05-1.98 (m, 2H), 1.66-1.58 (m, |
| | 2H), 1.36-1.30 (m, 3H) |
| Compound | 1H NMR (400 MHz, METHANOL-d4) δ ppm 5.22-5.11 (m, 1H), |
| 310 | 5.00-4.94 (m, 1H), 4.86-4.73 (m, 2H), 4.51-4.44 (m, 1H), 4.18- |
| | 4.11 (m, 1H), 3.90-3.69 (m, 14H), 3.66-3.53 (m, 6H), 3.41 (s, 3H), |
| | 3.37 (s, 6H), 3.05-2.98 (m, 3H), 2.07-2.00 (m, 2H), 1.69-1.58 (m, |
| | 2H), 1.34 (d, J = 6.4 Hz, 3H) |
| Compound | 1H NMR (400 MHz, METHANOL-d4) δ ppm 4.81-4.59 (m, 2H), |
| 311 | 4.56-4.16 (m, 2H), 4.01-3.72 (m, 13H), 3.71-3.46 (m, 7H), 3.41 |
| | (s, 2H), 3.40-3.34 (m, 7H), 3.03-2.95 (m, 3H), 2.19-1.99 (m, 2H), |
| | 1.81-1.64 (m, 2H), 1.46-1.33 (m, 6H) |
| Compound | 1H NMR (400 MHz, METHANOL-d4) δ ppm 4.77-4.66 (m, 2H), |
| 312 | 4.57-4.46 (m, 2H), 3.78-3.65 (m, 14H), 3.59-3.46 (m, 7H), 3.36- |
| | 3.34 (m, 3H), 3.31 (s, 6H), 2.92-2.89 (m, 3H), 2.02-1.94 (m, 2H), |
| | 1.66-1.51 (m, 2H), 1.34-1.31 (m, 6H) |
| Compound | 1H NMR (400 MHz, CHLOROFORM-d) δ ppm 4.35-4.17 (m, 2H), |
| 313 | 4.03-3.87 (m, 6H), 3.78 (br s, 10H), 3.69-3.56 (m, 6H), 3.50 (br d, |
| | J = 9.8 Hz, 3H), 3.38 (d, J = 6.0 Hz, 9H), 3.03-2.90 (m, 1H), 2.25- |
| | 2.16 (m, 1H), 2.02 (br d, J = 4.6 Hz, 3H), 1.86 (br d, J = 5.4 Hz, 3H), |
| | 1.56-1.45 (m, 1H) |
| Compound | 1H NMR (400 MHz, CHLOROFORM-d) δ ppm 5.49-5.43 (m, 2H), |
| 314 | 4.57-4.50 (m, 2H), 4.10-4.05 (m, 1H), 4.05-3.95 (m, 7H), 3.90- |
| | 3.80 (m, 6H), 3.75-3.65 (m, 4H), 3.65-3.55 (m, 4H), 3.53-3.45 (m, |
| | 3H), 3.39 (s, 3H), 3.35 (s, 6H), 2.85 (s, 1H), 2.16-2.13 (m, 1H), |
| | 2.04-2.02 (m, 3H), 2.01-2.00 (m, 1H), 1.73-1.72 (m, 2H), 1.58- |
| | 1.53 (m, 1H) |
| Compound | 1H NMR (400 MHz, METHANOL-d4) δ ppm 4.79-4.69 (m, 2H), |
| 315 | 4.54 (br s, 2H), 3.85-3.72 (m, 13H), 3.71-3.59 (m, 7H), 3.56-3.50 |
| | (m, 3H), 3.42-3.40 (m, 3H), 3.38 (s, 6H), 2.84-2.77 (m, 1H), 2.10- |
| | 1.97 (m, 2H), 1.69-1.57 (m, 2H), 0.88-0.82 (m, 2H), 0.77-0.72 |
| | (m, 2H) |
| Compound | 1H NMR (400 MHz, METHANOL-d4) δ ppm 4.78-4.74 (m, 1H), |
| 316 | 4.63-4.58 (m, 1H), 4.47 (br t, J = 5.3 Hz, 2H), 3.84-3.73 (m, 13H), |
| | 3.71-3.58 (m, 7H), 3.56-3.50 (m, 3H), 3.41 (s, 3H), 3.37 (s, 6H), |
| | 2.84-2.74 (m, 1H), 2.09-1.97 (m, 2H), 1.68-1.57 (m, 2H), 0.87- |
| | 0.82 (m, 2H), 0.77-0.73 (m, 2H) |
| Compound | 1H NMR (400 MHz, CHLOROFORM-d) δ ppm 4.87-4.54 (m, 2H), |
| 318 | 4.18-3.93 (m, 4H), 3.86-3.68 (m, 8H), 3.65-3.46 (m, 10H), 3.41- |
| | 3.39 (m, 3H), 3.39-3.33 (m, 12H), 2.10-1.95 (m, 2H), 1.82-1.64 |
| | (m, 9H) |
| Compound | 1H NMR (400 MHz, METHANOL-d4) δ ppm 4.73-4.35 (m, 4H), |
| 319 | 4.32-4.06 (m, 3H), 4.02-3.74 (m, 7H), 3.70-3.54 (m, 12H), 3.42- |
| | 3.37 (m, 12H), 3.03 (s, 3H), 1.87-1.66 (m, 6H) |
| Compound | 1H NMR (400 MHz, METHANOL-d4) δ ppm 4.72-4.48 (m, 4H), |
| 320 | 3.91-3.74 (m, 8H), 3.68-3.60 (m, 11H), 3.58-3.54 (m, 3H), 3.53- |
| | 3.46 (m, 3H), 3.41-3.36 (m, 15H), 3.03 (s, 3H) |
| Compound | 1H NMR (400 MHz, CHLOROFORM-d) δ ppm 4.82 (br s, 2H), |
| 321 | 4.48-4.39 (m, 2H), 4.32 (br t, J = 5.4 Hz, 2H), 3.91-3.82 (m, 8H), |
| | 3.78-3.61 (m, 10H), 3.48 (br s, 3H), 3.38 (s, 3H), 3.05-2.96 (m, |
| | 3H), 2.06-1.95 (m, 2H), 1.74-1.67 (m, 2H). |
| Compound | 1H NMR (400 MHz, CHLOROFORM-d) δ ppm 4.81 (br s, 2H), |
| 322 | 4.64-4.29 (m, 4H), 4.10-3.87 (m, 4H), 3.79 (br s, 8H), 3.65-3.55 |
| | (m, 6H), 3.48 (br s, 1H), 3.42-3.33 (m, 9H), 2.92 (br s, 2H), 2.45 (s, |
| | 3H), 2.00 (br s, 2H), 1.71 (br s, 2H) |
| Compound | 1H NMR (400 MHz, CHLOROFORM-d) δ ppm 5.40 (br s, 2H), |
| 323 | 4.68-4.28 (m, 6H), 4.00-3.72 (m, 14H), 3.61-3.47 (m, 5H), 3.37 |
| | (d, J = 18.0 Hz, 9H), 2.37 (s, 3H), 2.07-1.96 (m, 2H), 1.76-1.71 (m, |
| | 2H) |
| Compound | 1H NMR (400 MHz, CHLOROFORM-d) δ ppm 7.30-7.27 (m, 1H), |
| 324 | 7.11 (d, J = 7.6 Hz, 1H), 7.02 (d, J = 9.6 Hz, 1H), 6.96-6.91 (m, |
| | 1H), 4.75 (br s, 2H), 3.92-3.90 (m, 5H), 3.81-3.73 (m, 8H), 3.50- |
| | 3.40 (m, 5H), 3.39 (s, 3H), 3.31 (m, 6H), 2.03-1.99 (m, 3H), 1.74- |
| | 1.71 (m, 4H) |
| Compound | 1H NMR (400 MHz, CHLOROFORM-d) δ ppm 7.70-7.58 (m, 2H), |
| 325 | 7.56-7.53 (m, 1H), 7.47-7.44 (m, 1H), 4.80 (br s, 2H), 3.93-3.92 |
| | (m, 5H), 3.83-3.71 (m, 8H), 3.57-3.50 (m, 3H), 3.40 (m, 6H), 3.30 |
| | (br s, 4H), 2.02-2.01 (m, 2H), 1.75-1.61 (m, 8H) |
| Compound | 1H NMR (400 MHz, CHLOROFORM-d) δ ppm 4.84-4.33 (m, 6H), |
| 326 | 3.90-3.83 (m, 4H), 3.82-3.62 (m, 9H), 3.62-3.42 (m, 6H), 3.39 (s, |
| | 3H), 3.36 (s, 6H), 2.04-1.96 (m, 2H), 1.74-1.64 (m, 2H) |
| Compound | 1H NMR (400 MHz, CHLOROFORM-d) δ ppm 4.91-4.67 (m, 1H), |
| 327 | 4.64-4.36 (m, 5H), 4.27-4.11 (m, 3H), 3.79-3.72 (m, 6H), 3.71- |
| | 3.61 (m, 2H), 3.60-3.55 (m, 5H), 3.52-3.40 (m, 5H), 3.39 (s, 3H), |
| | 3.36 (s, 6H), 3.02 (s, 3H), 2.08-1.94 (m, 2H), 1.83-1.71 (m, 2H), |
| | 1.24 (s, 3H), 1.22 (s, 3H) |
| Compound | 1H NMR (400 MHz, CHLOROFORM-d) δ ppm 1.02-1.21 (m, 6 H) |
| 328 | 1.64-1.77 (m, 2 H) 1.95-2.05 (m, 2 H) 3.00 (s, 3 H) 3.22-3.34 (m, |
| | 6 H) 3.35-3.39 (m, 3 H) 3.44-3.51 (m, 3 H) 3.52-4.05 (m, 16 H) |
| | 4.13-4.94 (m, 6 H) |
| Compound | 1H NMR (400 MHz, CHLOROFORM-d) δ ppm 4.87-4.59 (m, 3H), |
| 329 | 4.23 (br s, 2H), 3.85-3.40 (m, 22H), 3.36 (d, J = 17.0 Hz, 9H), 3.02 |
| | (s, 3H), 2.02 (br s, 2H), 1.96-1.78 (m, 8H), 1.73 (br s, 2H). |
| Compound | 1H NMR (400 MHz, CHLOROFORM-d) δ ppm 4.63 (br s, 2H), |
| 330 | 4.13 (br s, 2H), 3.68 (br s, 5H), 3.64-3.45 (m, 12H), 3.39 (br d, |
| | J = 7.9 Hz, 11H), 3.34 (s, 6H), 2.03 (br s, 4H), 1.86 (br s, 12H) |
| Compound | 1H NMR (400 MHz, CHLOROFORM-d) δ ppm 4.61 (br s, 5H), |
| 331 | 3.91 (br t, J = 6.5 Hz, 2H), 3.84-3.69 (m, 6H), 3.69-3.43 (m, 12H), |
| | 3.43-3.28 (m, 12H), 3.03 (s, 3H), 2.72 (br t, J = 6.7 Hz, 2H), 2.00 (br |
| | d, J = 12.3 Hz, 2H), 1.74-1.60 (m, 2H) |
| Compound | 1H NMR (400 MHz, CHLOROFORM-d) δ ppm 4.90 (s, 2H), 4.76- |
| 332 | 4.57 (m, 2H), 4.33 (br t, J = 5.2 Hz, 2H), 3.92 (br t, J = 6.7 Hz, 2H), |
| | 3.80-3.71 (m, 7H), 3.63-3.55 (m, 7H), 3.53-3.47 (m, 3H), 3.40 (s, |
| | 3H), 3.36 (s, 3H), 3.36-3.34 (m, 6H), 3.01 (s, 3H), 2.76-2.69 (m, |
| | 2H), 2.01 (td, J = 3.2, 9.2 Hz, 2H), 1.73-1.64 (m, 2H) |
| Compound | 1H NMR (400 MHz, CHLOROFORM-d) δ ppm 7.96 (br d, J = 8.1 |
| 333 | Hz, 2H), 7.29 (br s, 2H), 4.94 (br s, 2H), 4.62-4.25 (m, 6H), 3.80 (br |
| | s, 9H), 3.60 (br d, J = 5.5 Hz, 8H), 3.37 (s, 9H), 3.31 (s, 3H), 3.00 (br |
| | s, 3H), 1.74-1.66 (m, 2H), 1.38-1.24 (m, 2H) |
| Compound | 1H NMR (400 MHz, CHLOROFORM-d) δ ppm 10.45-10.24 (m, |
| 334 | 1H), 4.76-4.45 (m, 5H), 4.05-3.87 (m, 3H), 3.85-3.74 (m, 4H), |
| | 3.69 (br s, 4H), 3.58 (br s, 6H), 3.47 (br d, J = 2.0 Hz, 3H), 3.40 (s, |
| | 6H), 3.37 (s, 6H), 3.01 (s, 3H), 2.78 (br s, 6H), 2.70-2.54 (m, 2H), |
| | 2.02 (br s, 2H), 1.74-1.65 (m, 2H) |
| Compound | 1H NMR (400 MHz, CHLOROFORM-d) δ ppm 7.95 (d, J = 8.0 Hz, |
| 335 | 2H), 7.29 (d, J = 8.0 Hz, 2H), 4.96 (s, 2H), 4.87-4.44 (m, 6H), 4.39- |
| | 4.15 (m, 2H), 3.89-3.74 (m, 6H), 3.63-3.55 (m, 6H), 3.49-3.41 |
| | (m, 2H), 3.35 (s, 6H), 3.31 (s, 3H), 3.00 (s, 3H), 2.92-2.54 (m, 2H), |
| | 2.43 (s, 3H) |
| Compound | 1H NMR (400 MHz, CHLOROFORM-d) δ ppm 7.87 (t, J = 7.8 Hz, |
| 336 | 1H), 7.06 (d, J = 8.2 Hz, 1H), 6.99 (d, J = 11.8 Hz, 1H), 4.89 (s, 2H), |
| | 4.53 (br s, 6H), 3.80-3.73 (m, 6H), 3.63-3.53 (m, 8H), 3.46 (br d, |
| | J = 1.2 Hz, 3H), 3.36 (s, 9H), 3.30 (s, 3H), 3.01 (s, 3H), 2.00-1.78 (m, |
| | 2H), 1.67-1.46 (m, 2H) |
| Compound | 1H NMR (400 MHz, CHLOROFORM-d) δ ppm 10.82-10.59 (m, |
| 337 | 1H), 8.08 (d, J = 7.8 Hz, 1H), 7.02-6.96 (m, 2H), 4.91 (s, 2H), 4.71- |
| | 4.32 (m, 6H), 4.04 (s, 3H), 3.84-3.74 (m, 6H), 3.67-3.54 (m, 8H), |
| | 3.50-3.42 (m, 3H), 3.37-3.31 (m, 12H), 3.01 (s, 3H), 1.95-1.78 |
| | (m, 2H), 1.68 (br s, 2H) |
| Compound | 1H NMR (400 MHz, CHLOROFORM-d) δ ppm 7.92 (br d, J = 8.6 |
| 338 | Hz, 1H), 7.12-7.08 (m, 2H), 4.89 (br s, 2H), 4.70-4.40 (m, 6H), |
| | 3.76 (br s, 6H), 3.63-3.53 (m, 8H), 3.48-3.40 (m, 3H), 3.36 (s, |
| | 9H), 3.32 (s, 3H), 3.01 (s, 3H), 2.59 (s, 3H), 1.96-1.78 (m, 2H), |
| | 1.65-1.47 (m, 2H) |
| Compound | 1H NMR (400 MHz, CHLOROFORM-d) δ ppm 7.76-7.68 (m, 1H), |
| 339 | 7.62 (s, 1H), 7.44 (d, J = 7.9 Hz, 1H), 4.90 (s, 2H), 4.68-4.45 (m, |
| | 4H), 3.87-3.70 (m, 8H), 3.64-3.54 (m, 8H), 3.45 (br s, 3H), 3.36 |
| | (s, 9H), 3.30 (s, 3H), 3.00 (s, 3H), 2.08-1.76 (m, 2H), 1.71-1.40 |
| | (m, 2H) |
| Compound | 1H NMR (400 MHz, CHLOROFORM-d) δ ppm 6.80 (br d, J = 8.8 |
| 340 | Hz, 2H), 4.78 (br s, 2H), 4.67-4.07 (m, 6H), 3.83-3.72 (m, 6H), |
| | 3.64-3.52 (m, 8H), 3.45 (br d, J = 1.0 Hz, 3H), 3.39-3.30 (m, 12H), |
| | 3.00 (br s, 3H), 2.03-1.85 (m, 2H), 1.70-1.49 (m, 2H) |

### II. Biology Examples

### Example II.1. Assay for ENT1 Activity

### Example II.1.a Binding Assay

Purpose. The present assay aims at showing that the compounds of the present invention can bind to human ENT1. The principle of the assay is a competition between the compounds of this invention and Sahenta-DY647, an ENT1 inhibitor that emits fluorescence (Ex=630 nm, Em=670 nm). By measuring the fluorescence at the end of the assay we could assess the binding potency of the compounds of the present invention.

Method. JAR cells expressing ENT1 were bought from ATCC® (HTB-144TM). Cells were cultured in RPMI 1640 medium (LONZA®, #BE12-702F/U1) supplemented with 10% FBS (GIBCO®, #10270-106), 10 mM Hepes (LONZA®, #BE17-737E), 1 mM Sodium Pyruvate (LONZA®, #BE13-115E) and 2% Penicillin/Streptomycin (LONZA®, #DE17-603E) at 37° C. and 5% C02.

The assay was conducted on the following buffer: HBSS (LONZA®, #LO-527F) supplemented with 10 mM Hepes (LONZA®, #BE17-737E) and 0.1% BSA (Miltenyi®, #130-091-376) on the day of the assay.

JAR cells were resuspended in the described buffer. Compounds of the present invention and Sahenta-DY647 were diluted 200× in the described buffer.

A total of 50 000 cells were pre-incubated for 30 min at 4° C. with the compounds of the present invention before adding the corresponded IC90 of Sahenta-DY647 (100 nM) and incubate once more for 30 min at 4° C. The total volume of the reaction was 100 μL (50 μL of cells, 25 μL of the compounds of the present invention and 25 μL of Sahenta-DY647) in a 96 well plate, U-bottom (Greiner®, #650-180). The plates were washed 2× by centrifugation (4 min, 400 rcf at 4° C.) in the same buffer. Cells were re-suspended in 70 μL of the buffer and 50 μL was transferred to a Black 384 Optiplate (PerkinElmer®, #6007279). Fluorescence (Ex=630 nm, Em=670 nm) was acquired on a Spectramax i3x (Molecular Devices®).

Results. Results obtained from this protocol are summarized in Table 5.

### Example II.1.b Functional Assay: Uridine Transport Inhibition Assay

Purpose. The aim of this study was to determine the potency of equilibrative nucleoside transporter 1 (ENT1) inhibitors by measuring ENT1-mediated transport is the cellular uptake assay. The human ENT1 transporter can be stably expressed in Madin-Darby Canine Kidney II (MDCKII) cells via transduction. Uridine is efficiently transported by ENT1 and is used as probe in the assay as 3H-uridine. The interaction is detected as the modulation of the initial rate of 3H-uridine transport by human ENT1 into MDCKII-ENT1-LV cells stably expressing ENT1 uptake transporter.

Results. Results obtained from this protocol are summarized in Table 6.

Assay Parameters

| | Incubation | Probe | Reference | Signal/ |
|---|---|---|---|---|
| | time (min) and | substrate | inhibitor | Noise |
| Transporter | temperature | (concentration) | (concentration) | ratio |
| ENT1 | 1 min at 25° C. | Uridine | Dilazep | >3 |
| | | (1 μM) | (2 μM) | |

### Example II.1.c Functional Assay: T Cell Proliferation Assay

Purpose. The aim of this study was to determine the potency of equilibrative nucleoside transporter 1 (ENT1) inhibitors to rescue proliferation by stimulated primary human T cells incubated in the presence of 100 uM Adenosine triphosphate (ATP) with:
Condition A: X-VIVO15
Condition B: X-VIVO15, 2% Human Serum Albumine (HSA) and 0.1% α-1-Acid Glycoprotein (AAG)

Materials

| Product | Source | Cat number |
|---|---|---|
| RPMI 1640 | LONZA | BE12-702F/U1 |
| FBS | GIBCO | 10270-106 |
| X-VIVO15 | Lonza | BE02-060Q |
| Human Serum Albumine | Sigma-Aldrich | A1653 |
| (HSA) | | |
| α₁-Acid Glycoprotein | Sigma-Aldrich | G9885 |
| (AAG) | | |
| Dynabeads CD3/28 | Thermo Fisher | 11132D |
| activation beads | Scientific | |
| CFDA, SE | Life Technologies | C1157 |
| EOS502085_1 | Tocris | 0691 |
| (Dipyridamole) | | |
| ATP | Sigma Aldrich | A6419-1G |

Method. Cryopreserved purified human CD3+ T cells were thawed and washed twice with RPMI1640 medium, UltraGlutamine containing 10% hiFBS.

Cells were suspended in PBS containing 10% hiFBS. Cells were stained with CFSE by adding 2 μM solution in PBS, to get a final 1 μM CFSE solution. Cells were incubated while rotating for 5 minutes. Reaction was stopped by adding PBS with 10% FBS and cells were centrifuged for 5 minutes at 1500 rpm.

Cells were resuspended at 1.6×106 cells/mL, either in X-VIVO15 medium or in 4% Human Serum Albumin and 0.2% α-1-Acid Glycoprotein. 50 μL of cell suspension (8×104 T cells) was added to wells of sterile round-bottom 96-well plates. Cells were activated by adding 50 μL of anti-CD3 anti-CD28 coated microbeads, suspended either in XVIVO-15 medium or in 4% HSA and 0.2% α-1-Acid Glycoprotein, at a ratio of one microbead per two cells.

Serial dilutions of the ENT1 inhibitors were prepared in X-VIVO15 from 10 mM stock solutions in DMSO, and 50 μL was added to the wells.

ATP powder was diluted in X-VIVO15, and 50 μL of this compound was added to the wells to reach a final assay concentration of 100 μM. Final volume of 200 μL.

The experiments were also performed in 384 well plates—all volumes reduced by a factor of 4 (12.5 L) with a final volume of 50 μL.

Experiments were performed in duplicate. The cells were placed in a 37° C. humidified tissue culture incubator with 5% CO2 for 72 hours for 96 well plates, 96 hours for 384 well plates. After 72 or 96 hours, proliferation was measured determined by CFSE dilution via flow cytometry.

Results. Results are detailed below in Table 6. Compounds of the invention have good ENT1 inhibitory properties.

### Example II.2. AAG Binding Assay—HPLC Assay

Purpose. The present assay aims at showing that the compounds of the present invention do not eagerly bind to human alpha 1-acid glycoprotein (AAG), contrary to what was previously observed for dipyridamole. The principle of the assay is a liquid chromatography on a human AAG protein immobilized onto a silica support for separations of compounds following their affinity to AAG protein. By measuring the retention time, the binding to AAG can be determined relatively to the references.

Method. The instrument used for HPLC is a SHIMADZU Nexera X2(LC-30AD) equipped with a column CHIRALPAK® AGP 3 mm×150 mm, 5 μm. Mobile Phase A: 50 mM Ammonium acetate in deionized water; Mobile Phase B: Isopropanol; Column Oven: 8° C.; PDA(nm): 230; Flow Rate (mL/min): 0.2. Test Concentration (μmol/L): 100 (from 10 mmol/L DMSO solution diluted 100 folds by 50 mM Ammonium acetate in deionized water: Isopropanol (1:1)). Generic Gradient: Time (min): 0.01; 5; 13; 14; 20.

Sample order: Dipyridamole, Propranolol, Compound 2 then 15 tested samples then references again.

References: Dipyridamole (rt=8.64 min), Propranolol (rt=14.05 min), Compound 2 (rt=5.2 min).

Results. Results are detailed below in Table 5. Compounds of the invention present a low binding to AAG, compared to dipyridamole.

### Example II.3. Results of Ent1 Inhibition and AAG Binding Assays

Results: The unbound fraction of the tested compound was estimated by a chromatographic method, as well, the potency as been determined as is reported in Table 5. The compounds of the invention present a combined improved fraction (unbound fraction), compared to dipyridamole, while the potency against ENT1 has been maintained or improved.

The IC₅₀ has been binned following the ranges: IC₅₀ below 0.1 μM: +++; IC₅₀ between 0.1 and 0.5 μM: ++; IC₅₀ between 0.5 and 1 μM: +.

The AAG binding in HPLC has been binned following the range: rt below 6 min: −−, rt between 6 and 8 min: −, NA: not available.

TABLE 5

| Compounds | Binding Assay IC₅₀ | AAG HPLC |
|---|---|---|
| Compound 1 | +++ | NA |
| Compound 2 | ++ | −− |
| Compound 3 | ++ | − |
| Compound 4 | ++ | NA |
| Compound 5 | +++ | NA |
| Compound 6 | ++ | NA |
| Compound 7 | +++ | NA |
| Compound 8 | +++ | NA |
| Compound 9 | ++ | NA |
| Compound 10 | ++ | NA |
| Compound 11 | +++ | NA |
| Compound 12 | +++ | NA |
| Compound 13 | +++ | NA |
| Compound 14 | + | −− |
| Compound 15 | +++ | NA |
| Compound 16 | + | NA |
| Compound 19 | ++ | − |
| Compound 27 | + | −− |
| Compound 30 | + | NA |
| Compound 31 | + | −− |
| Compound 32 | + | −− |
| Compound 33 | +++ | − |
| Compound 34 | + | −− |
| Compound 37 | + | NA |
| Compound 38 | ++ | NA |
| Compound 39 | +++ | NA |
| Compound 44 | + | NA |
| Compound 45 | ++ | −− |
| Compound 46 | ++ | −− |
| Compound 47 | ++ | −− |
| Compound 48 | +++ | −− |
| Compound 49 | +++ | −− |
| Compound 50 | ++ | −− |
| Compound 51 | + | NA |
| Compound 52 | + | −− |
| Compound 53 | + | −− |
| Compound 54 | +++ | NA |
| Compound 55 | ++ | NA |
| Compound 56 | ++ | NA |
| Compound 57 | + | NA |
| Compound 58 | + | NA |
| Compound 59 | ++ | NA |
| Compound 60 | + | NA |
| Compound 61 | + | NA |
| Compound 62 | + | NA |
| Compound 63 | ++ | NA |
| Compound 64 | ++ | NA |
| Compound 65 | + | NA |
| Compound 66 | + | NA |
| Compound 67 | + | −− |
| Compound 68 | +++ | −− |
| Compound 69 | + | −− |
| Compound 70 | + | −− |
| Compound 71 | ++ | −− |
| Compound 72 | +++ | NA |
| Compound 73 | ++ | − |
| Compound 74 | ++ | NA |
| Compound 75 | ++ | NA |
| Compound 76 | + | −− |
| Compound 77 | +++ | NA |
| Compound 78 | + | −− |
| Compound 79 | +++ | NA |
| Compound 80 | ++ | NA |
| Compound 81 | +++ | −− |
| Compound 82 | ++ | −− |
| Compound 83 | + | − |
| Compound 84 | + | NA |
| Compound 85 | +++ | −− |
| Compound 86 | ++ | −− |
| Compound 87 | ++ | −− |
| Compound 88 | + | NA |
| Compound 89 | ++ | −− |
| Compound 90 | ++ | −− |
| Compound 91 | ++ | −− |
| Compound 92 | ++ | NA |
| Compound 93 | + | −− |
| Compound 94 | + | −− |
| Compound 95 | + | − |
| Compound 96 | ++ | NA |
| Compound 97 | +++ | −− |
| Compound 98 | ++ | −− |
| Compound 99 | ++ | NA |
| Compound 100 | ++ | − |
| Compound 101 | + | −− |
| Compound 102 | +++ | −− |
| Compound 103 | ++ | − |
| Compound 104 | ++ | − |
| Compound 105 | ++ | NA |
| Compound 106 | ++ | −− |
| Compound 107 | ++ | −− |
| Compound 108 | +++ | −− |
| Compound 109 | ++ | −− |
| Compound 110 | +++ | −− |
| Compound 111 | +++ | −− |
| Compound 112 | ++ | NA |
| Compound 113 | ++ | NA |
| Compound 114 | +++ | NA |
| Compound 115 | ++ | NA |
| Compound 116 | +++ | −− |
| Compound 117 | + | NA |
| Compound 118 | ++ | NA |
| Compound 119 | + | NA |
| Compound 120 | + | NA |
| Compound 121 | + | NA |
| Compound 122 | ++ | NA |
| Compound 123 | ++ | NA |
| Compound 124 | ++ | NA |
| Compound 125 | ++ | NA |
| Compound 126 | + | NA |
| Compound 127 | ++ | − |
| Compound 128 | + | NA |
| Compound 129 | ++ | NA |
| Compound 130 | + | −− |
| Compound 131 | ++ | NA |
| Compound 132 | + | NA |
| Compound 133 | + | NA |
| Compound 134 | + | − |
| Compound 135 | + | NA |
| Compound 136 | +++ | NA |
| Compound 137 | +++ | NA |
| Compound 138 | +++ | NA |
| Compound 139 | +++ | NA |
| Compound 140 | ++ | NA |
| Compound 141 | +++ | NA |
| Compound 142 | +++ | NA |
| Compound 143 | +++ | NA |
| Compound 144 | ++ | NA |
| Compound 145 | +++ | NA |
| Compound 146 | ++ | NA |
| Compound 147 | + | NA |
| Compound 148 | ++ | NA |
| Compound 149 | +++ | NA |
| Compound 150 | +++ | NA |
| Compound 151 | ++ | NA |
| Compound 152 | +++ | NA |
| Compound 153 | +++ | NA |
| Compound 154 | +++ | NA |
| Compound 155 | +++ | NA |
| Compound 156 | ++ | NA |
| Compound 157 | +++ | NA |
| Compound 158 | ++ | NA |
| Compound 159 | ++ | NA |
| Compound 160 | + | NA |
| Compound 161 | ++ | NA |
| Compound 162 | + | NA |
| Compound 163 | ++ | NA |
| Compound 164 | + | NA |
| Compound 165 | +++ | NA |
| Compound 166 | ++ | NA |
| Compound 167 | ++ | NA |
| Compound 168 | +++ | NA |
| Compound 169 | +++ | NA |
| Compound 170 | +++ | NA |
| Compound 171 | +++ | NA |
| Compound 172 | ++ | NA |
| Compound 173 | ++ | NA |
| Compound 174 | ++ | NA |
| Compound 175 | + | NA |
| Compound 176 | + | NA |
| Compound 177 | + | NA |
| Compound 178 | + | NA |
| Compound 179 | ++ | NA |
| Compound 180 | + | NA |
| Compound 181 | + | NA |
| Compound 182 | + | NA |
| Compound 183 | +++ | NA |
| Compound 184 | +++ | NA |
| Compound 185 | +++ | NA |
| Compound 186 | +++ | NA |
| Compound 187 | ++ | NA |
| Compound 188 | ++ | NA |
| Compound 189 | ++ | NA |
| Compound 190 | +++ | NA |
| Compound 191 | ++ | NA |
| Compound 192 | ++ | NA |
| Compound 193 | ++ | NA |
| Compound 195 | ++ | − |
| Compound 196 | ++ | NA |
| Compound 197 | + | NA |
| Compound 198 | ++ | NA |
| Compound 199 | +++ | NA |
| Compound 200 | +++ | NA |
| Compound 201 | ++ | NA |
| Compound 202 | + | NA |
| Compound 203 | ++ | NA |
| Compound 204 | ++ | NA |
| Compound 205 | +++ | NA |
| Compound 206 | +++ | NA |
| Compound 207 | +++ | NA |
| Compound 208 | +++ | NA |
| Compound 209 | +++ | NA |
| Compound 210 | +++ | NA |
| Compound 211 | +++ | NA |
| Compound 212 | + | NA |
| Compound 213 | +++ | NA |
| Compound 214 | ++ | NA |
| Compound 215 | ++ | NA |
| Compound 216 | ++ | NA |
| Compound 217 | ++ | NA |
| Compound 218 | ++ | NA |
| Compound 219 | ++ | NA |
| Compound 220 | +++ | NA |
| Compound 221 | +++ | NA |
| Compound 222 | +++ | NA |
| Compound 223 | +++ | NA |
| Compound 224 | +++ | NA |
| Compound 225 | +++ | NA |
| Compound 226 | ++ | NA |
| Compound 227 | +++ | NA |
| Compound 228 | ++ | NA |
| Compound 229 | ++ | NA |
| Compound 230 | +++ | NA |
| Compound 231 | ++ | NA |
| Compound 232 | +++ | NA |
| Compound 233 | +++ | NA |
| Compound 234 | +++ | NA |
| Compound 235 | +++ | NA |
| Compound 236 | +++ | NA |
| Compound 237 | +++ | NA |
| Compound 238 | +++ | NA |
| Compound 239 | +++ | NA |
| Compound 240 | ++ | NA |
| Compound 241 | ++ | NA |
| Compound 242 | + | NA |
| Compound 243 | +++ | NA |
| Compound 244 | ++ | NA |
| Compound 245 | + | NA |
| Compound 246 | + | NA |
| Compound 247 | ++ | NA |
| Compound 248 | ++ | NA |
| Compound 249 | ++ | NA |
| Compound 250 | ++ | NA |
| Compound 251 | ++ | NA |
| Compound 252 | +++ | NA |
| Compound 253 | ++ | NA |
| Compound 254 | +++ | NA |
| Compound 255 | +++ | NA |
| Compound 256 | +++ | NA |
| Compound 257 | +++ | NA |
| Compound 258 | +++ | NA |
| Compound 259 | +++ | NA |
| Compound 260 | +++ | NA |
| Compound 261 | +++ | NA |
| Compound 262 | ++ | NA |
| Compound 263 | ++ | NA |
| Compound 264 | NA | NA |
| Compound 265 | NA | NA |
| Compound 266 | +++ | NA |
| Compound 267 | ++ | NA |
| Compound 268 | +++ | NA |
| Compound 269 | +++ | NA |
| Compound 270 | ++ | NA |
| Compound 271 | + | NA |
| Compound 272 | ++ | NA |
| Compound 273 | NA | NA |
| Compound 274 | NA | NA |
| Compound 275 | +++ | NA |
| Compound 276 | ++ | NA |
| Compound 277 | ++ | NA |
| Compound 278 | ++ | NA |
| Compound 279 | ++ | NA |
| Compound 280 | ++ | NA |
| Compound 281 | + | NA |
| Compound 282 | ++ | NA |
| Compound 283 | + | NA |
| Compound 284 | + | NA |
| Compound 285 | ++ | NA |
| Compound 286 | ++ | NA |
| Compound 287 | ++ | NA |
| Compound 288 | ++ | NA |
| Compound 289 | +++ | NA |
| Compound 290 | + | NA |
| Compound 291 | + | NA |
| Compound 292 | ++ | NA |
| Compound 293 | ++ | NA |
| Compound 294 | +++ | NA |
| Compound 295 | ++ | NA |
| Compound 296 | ++ | NA |
| Compound 297 | ++ | NA |
| Compound 298 | + | NA |
| Compound 299 | ++ | NA |
| Compound 300 | ++ | NA |
| Compound 301 | +++ | NA |
| Compound 302 | +++ | NA |
| Compound 303 | +++ | NA |
| Compound 304 | +++ | NA |
| Compound 305 | +++ | NA |
| Compound 306 | ++ | NA |
| Compound 307 | +++ | NA |
| Compound 308 | +++ | NA |
| Compound 309 | ++ | NA |
| Compound 310 | + | NA |
| Compound 311 | +++ | NA |
| Compound 312 | ++ | NA |
| Compound 313 | ++ | NA |
| Compound 314 | + | NA |
| Compound 315 | + | NA |
| Compound 316 | + | NA |
| Compound 318 | + | NA |
| Compound 319 | ++ | NA |
| Compound 320 | + | NA |
| Compound 321 | + | NA |
| Compound 322 | ++ | NA |
| Compound 323 | ++ | NA |
| Compound 324 | +++ | NA |
| Compound 325 | +++ | NA |
| Compound 326 | + | NA |
| Compound 327 | +++ | NA |
| Compound 328 | ++ | NA |
| Compound 329 | +++ | NA |
| Compound 330 | ++ | NA |
| Compound 331 | NA | NA |
| Compound 332 | NA | NA |
| Compound 333 | + | NA |
| Compound 334 | + | NA |
| Compound 335 | +++ | NA |
| Compound 336 | +++ | NA |
| Compound 337 | ++ | NA |
| Compound 338 | +++ | NA |
| Compound 339 | +++ | NA |
| Compound 340 | +++ | NA |

Discussion of Results in Table 6: The potency on two functional assays, and one in the challenging conditions where representative concentration of AAG in TME (tumor microenvironment) has been used, is reported in Table 6. The compounds of the invention present a maintained, or improved potency as compared to dipyridamole in all functional assays. In particular, the compounds of the invention present a significantly improved potency in T Cell proliferation assay in presence of AAG protein (condition B) as compared to dipyridamole, meaning these compounds demonstrated a significant higher potency in the TME conditions as compared to dipyridamole.

TABLE 6

| | Uridine | TCell | TCell |
|---|---|---|---|
| | transport | proliferation | proliferation |
| | inhibition | (Condition A) | (Condition B) |
| COMPOUNDS | assay IC50 | IC50 | IC50 |
| Compound 2 | +++ | ++ | ++ |
| Compound 68 | +++ | + | + |
| Compound 72 | +++ | ++ | ++ |
| Compound 81 | +++ | ++ | + |
| Compound 149 | +++ | ++ | ++ |
| Compound 171 | +++ | ++ | ++ |
| Compound 184 | +++ | +++ | +++ |
| Compound 186 | +++ | +++ | ++ |

### Example II.4. AAG Binding Assays—Dialysis Assay

Purpose. The aim of this assay is to determine the binding of a compound of interest to the human AAG protein using the robust method of dialysis.

Material. HT-Dialysis plate (Model HTD 96 b, Cat #1006) and the dialysis membrane (molecular weight cut off 12-14 kDa, Cat #1101) were purchased from HT Dialysis LLC (Gales Ferry, Conn.). The dialysis Buffer (100 mM Phosphate Buffered Saline, pH 7.4) has been prepared by dissolving 28.56 g Na2HPO4.12H2O (AR grade) and 3.12 g NaH2PO4.2H2O (AR grade) into a final volume of 1000 mL ultra pure water, mixed, and adjusted the resulting solution with 1% phosphoric acid or 1 M sodium hydroxide to pH 7.4±0.1. Stored the final solution in 2-8° C. refrigerator within an expiration of a month after preparation.

Method: The dialysis membrane is soaked in dialysis buffer at 4° C. overnight. The following day the membrane is separated into two strips and then soaked in ethanol: water (20:80 v:v) for 20 mins. Then the membrane was rinsed with ultra-pure water 3-4 times. Prepared 400 μM working solution by diluting the stock solution (10 mM) with appropriate volume of DMSO. 5 μL aliquots of each working solution were spiked into 1000 μL of blank protein (matrix) to achieve a 2 μM final concentration as loading solution. The plates of loading solution were mixed well. Aliquots of 150 μL of loading solution were loaded in triplicate to the donor side of each dialysis well and dialyzed against an equal volume of dialysis buffer. Then the plate was sealed and rotated at approximately 100 rpm in a humidified incubator with 5% CO2 at 37° C. for 4-hr. At the end of dialysis, aliquots of dialysate (50 μL) and retenate (50 μL) were removed into sample collection plates. Each sample was matched with opposite blank buffer or matrix to obtain a final volume of 100 μL in each well and participated with 300 μL of stop solution. All sample collection plates were shaken at 800 rpm for 5 min to mix samples and then centrifuged at 20° C., 4000 rpm for 20 min. Aliquots of supernatant (100 μL) in each well were transferred into new 96-well sample plates mixed with 100 μL ultra pure water before subjecting to LC-MS/MS analysis.

The concentration was calculated using the peak area ratio of analyte and internal standard.

Results: The unbound fraction of the tested compound was determined, as is reported in Table 7. The compounds of the invention present a more important free fraction (unbound fraction), compared to dipyridamole, being thus more bioavailable.

TABLE 7

| | Human alpha-1-acid |
|---|---|
| Tested | glycoprotein (AAG) |
| compound | (1 mg/mL) |
| Dipyridamol | 0% Unbound |
| Compound 2 | 24.6% Unbound |
| Compound 27 | 89.4% Unbound |

### Example II.5. PDEs Assays

Purpose. Evaluation of the effects of compounds on the activity of the following phosphodiesterases (PDEs) known to be an important off-targets of Dipyridamole.

Method.
Human phosphodiesterase-1B quantified by measuring the formation of 5′GMP from cGMP using a human recombinant enzyme expressed in Sf9 cells

The test compound (compound 72), reference compound or water (control) were added to a buffer containing 40 mM Tris/HCl (pH 7.4), 8 mM MgCl2 0.21% BSA, 0.5 μg/ml calmoduline and 0.5 mM CaCl₂), 1.125 μM cGMP and 0.027 μCi [3H]cGMP. Thereafter, the reaction was initiated by addition of the enzyme (about 0.2 U) and the mixture was incubated for 20 min at 22° C. For basal control measurements, the enzyme was omitted from the reaction mixture. Following incubation SPA beads were added. After 30 min at 22° C. under shaking, the amount of [3H]5′GMP was quantified with a scintillation counter (MicroBeta, Perkin Elmer). The results were expressed as a percent inhibition of the control enzyme activity. The standard inhibitory reference compound was Nitrendipine, which was tested in each experiment at several concentrations to obtain an inhibition curve from which its IC50 value was calculated.
Human phosphodiesterase-5 quantified by measuring the formation of 5′GMP from cGMP using an enzyme isolated from human platelets at Eurofins-Cerep.

The test compound (compound 72), reference compound or water (control) were added to a buffer containing 40 mM Tris/HCl (pH 7.8), 3 mM MgCl2, 1.4 mM DTT, 0.21% BSA, 200 mM NH4Cl, 1 μM cGMP and 0.1 μCi [3H]cGMP. Thereafter, the reaction was initiated by addition of the enzyme (final amount depending on the efficiency of the isolation) and the mixture was incubated for 60 min at 22° C. For basal control measurements, the enzyme was omitted from the reaction mixture. Following incubation SPA beads were added. After 20 min at 22° C. under shaking, the amount of [3H]5′GMP was quantified with a scintillation counter (MicroBeta, Perkin Elmer). The results were expressed as a percent inhibition of the control enzyme activity. The standard inhibitory reference compound was dipyridamole, which was tested in each experiment at several concentrations to obtain an inhibition curve from which its IC50 value was calculated.
Human phosphodiesterase-2A1 quantified by measuring the formation of 5′AMP from cAMP using a human recombinant enzyme expressed in Sf9 cells

The test compound (compound 72), reference compound or water (control) were added to a buffer containing 40 mM Tris/HCl (pH 7.4), 8 mM MgCl2 and 1.7 mM EGTA/NaOH, 1.8 μM cAMP and 1 μCi [3H]cAMP. Thereafter, the reaction was initiated by addition of the enzyme (about 2 ng) and the mixture was incubated for 15 min at 22° C. For basal control measurements, the enzyme was omitted from the reaction mixture. Following incubation SPA beads were added. After 30 min at 22° C. under shaking, the amount of [3H]5′AMP was quantified with a scintillation counter (MicroBeta, Perkin Elmer). The results were expressed as a percent inhibition of the control enzyme activity. The standard inhibitory reference compound was EHNA, which was tested in each experiment at several concentrations to obtain an inhibition curve from which its IC50 value was calculated.
Human phosphodiesterase-7A1 quantified by measuring the formation of 5′AMP from cAMP using a human recombinant enzyme expressed in Sf9 cells

The test compound (compound 72), reference compound or water (control) were added to a buffer containing 40 mM Tris/HCl (pH 7.4) and 8 mM MgCl2, 63 nM cAMP and 0.035 μCi [3H]cAMP. Thereafter, the reaction was initiated by addition of the enzyme (about 0.6 U) and the mixture was incubated for 20 min at 22° C. For basal control measurements, the enzyme was omitted from the reaction mixture. Following incubation SPA beads were added. After 30 min at 22° C. under shaking, the amount of [3H]5′AMP was quantified with a scintillation counter (MicroBeta, Perkin Elmer). The results were expressed as a percent inhibition of the control enzyme activity. The standard inhibitory reference compound was BRL50481, which was tested in each experiment at several concentrations to obtain an inhibition curve from which its IC50 value was calculated.
Human phosphodiesterase-8A1 quantified by measuring the formation of 5′AMP from cAMP using a human recombinant enzyme expressed in Sf9 cells

The test compound (compound 72), reference compound or water (control) were added to a buffer containing 40 mM Tris/HCl (pH 7.4) and 8 mM MgCl2, 180 nM cAMP and 0.1 μCi [3H]cAMP. Thereafter, the reaction was initiated by addition of the enzyme (about 0.8 U) and the mixture was incubated for 20 min at 22° C. For basal control measurements, the enzyme was omitted from the reaction mixture. Following incubation SPA beads were added. After 30 min at 22° C. under shaking, the amount of [3H]5′AMP was quantified with a scintillation counter (MicroBeta, Perkin Elmer). The results were expressed as a percent inhibition of the control enzyme activity. The standard inhibitory reference compound was trequinsin, which was tested in each experiment at several concentrations to obtain an inhibition curve from which its IC50 value was calculated.
Human phosphodiesterase-10A2 quantified by measuring the formation of 5′AMP from cAMP using a human recombinant enzyme expressed in Sf9 cells.

The test compound (compound 72), reference compound or water (control) were added to a buffer containing 40 mM Tris/HCl (pH 7.4) and 8 mM MgCl2, 180 nM cAMP and 0.1 μCi [3H]cAMP. Thereafter, the reaction was initiated by addition of the enzyme (about 0.8 U) and the mixture was incubated for 20 min at 22° C. For basal control measurements, the enzyme was omitted from the reaction mixture. Following incubation SPA beads were added. After 30 min at 22° C. under shaking, the amount of [3H]5′AMP was quantified with a scintillation counter (MicroBeta, Perkin Elmer). The results were expressed as a percent inhibition of the control enzyme activity. The standard inhibitory reference compound was papaverine, which was tested in each experiment at several concentrations to obtain an inhibition curve from which its IC50 value was calculated.
Human phosphodiesterase-3A quantified by measuring the formation of 5′AMP from cAMP using a human recombinant enzyme expressed in Sf9 cells

The test compound (compound 72), reference compound or water (control) were added to a buffer containing 40 mM Tris/HCl (pH 7.4) and 8 mM MgCl2, 450 nM cAMP and 0.25 μCi [3H]cAMP. Thereafter, the reaction was initiated by addition of the enzyme (about 4 ng) and the mixture was incubated for 15 min at 22° C. For basal control measurements, the enzyme was omitted from the reaction mixture. Following incubation SPA beads were added. After 30 min at 22° C. under shaking, the amount of [3H]5′AMP was quantified with a scintillation counter (Topcount, Packard). The results were expressed as a percent inhibition of the control enzyme activity. The standard inhibitory reference compound was milrinone, which was tested in each experiment at several concentrations to obtain an inhibition curve from which its IC50 value was calculated.
Human phosphodiesterase-11A quantified by measuring the formation of 5′AMP from cAMP using a human recombinant enzyme expressed in Sf9 cells.

The test compound (compound 72), reference compound or water (control) were added to a buffer containing 40 mM Tris/HCl (pH 7.4) and 8 mM MgCl2, 450 nM cAMP and 0.25 μCi [3H]cAMP. Thereafter, the reaction was initiated by addition of the enzyme (about 3 U) and the mixture was incubated for 20 min at 22° C. For basal control measurements, the enzyme was omitted from the reaction mixture. Following incubation SPA beads were added. After 30 min at 22° C. under shaking, the amount of [3H]5′AMP was quantified with a scintillation counter (MicroBeta, Perkin Elmer). The results were expressed as a percent inhibition of the control enzyme activity. The standard inhibitory reference compound was dipyridamole, which was tested in each experiment at several concentrations to obtain an inhibition curve from which its IC50 value was calculated.
Human phosphodiesterase-4A1A quantified by measuring the formation of 5′AMP from cAMP using a human recombinant enzyme expressed in Sf9 cells.

The test compound (compound 72), reference compound or water (control) were added to a buffer containing 40 mM Tris/HCl (pH 7.4) and 8 mM MgCl2, 450 nM cAMP and 0.25 μCi [3H]cAMP. Thereafter, the reaction was initiated by addition of the enzyme (about 10 U) and the mixture was incubated for 20 min at 22° C. For basal control measurements, the enzyme was omitted from the reaction mixture. Following incubation SPA beads were added. After 30 min at 22° C. under shaking, the amount of [3H]5′AMP was quantified with a scintillation counter (MicroBeta, Perkin Elmer). The results were expressed as a percent inhibition of the control enzyme activity. The standard inhibitory reference compound was Ro 20-1724, which was tested in each experiment at several concentrations to obtain an inhibition curve from which its IC50 value was calculated.
Human phosphodiesterase-4B1 quantified by measuring the formation of 5′AMP from cAMP using a human recombinant enzyme expressed in Sf9 cells.

The test compound (compound 72), reference compound or water (control) were added to a buffer containing 40 mM Tris/HCl (pH 7.4), 8 mM MgCl2 and 1.7 mM EGTA/NaOH, 450 nM cAMP and 0.25 μCi [3H]cAMP. Thereafter, the reaction was initiated by addition of the enzyme (about 1.2 U) and the mixture was incubated for 20 min at 22° C. For basal control measurements, the enzyme was omitted from the reaction mixture. Following incubation SPA beads were added. After 30 min at 22° C. under shaking, the amount of [3H]5′AMP was quantified with a scintillation counter (MicroBeta, Perkin Elmer). The results were expressed as a percent inhibition of the control enzyme activity. The standard inhibitory reference compound was Ro 20-1724, which was tested in each experiment at several concentrations to obtain an inhibition curve from which its IC50 value was calculated.
Human phosphodiesterase-4D2 quantified by measuring the formation of 5′AMP from cAMP using a human recombinant enzyme expressed in Sf9 cells.

The test compound (compound 72), reference compound or water (control) were added to a buffer containing 40 mM Tris/HCl (pH 7.4) and 8 mM MgCl2, 450 nM cAMP and 0.0125 μCi [3H]cAMP. Thereafter, the reaction was initiated by addition of the enzyme (about 1.5 U) and the mixture was incubated for 20 min at 22° C. For basal control measurements, the enzyme was omitted from the reaction mixture. Following incubation SPA beads were added. After 30 min at 22° C. under shaking, the amount of [3H]5′AMP was quantified with a scintillation counter (Topcount, Packard). The results were expressed as a percent inhibition of the control enzyme activity. The standard inhibitory reference compound was Ro 20-1724, which was tested in each experiment at several concentrations to obtain an inhibition curve from which its IC50 value was calculated.
Phosphodiesterase-6 quantified by measuring the formation of 5′GMP from cGMP using an enzyme isolated from bovine retinas at Eurofins-Cerep

The test compound (compound 72), reference compound or water (control) were added to a buffer containing 40 mM Tris/HCl (pH 7.8), 3 mM MgCl2, 1.4 mM DTT, 0.21% BSA, 200 mM NH4Cl, 2 μM cGMP and 0.05 μCi [3H]cGMP. Thereafter, the reaction was initiated by addition of the enzyme (final amount depending on the efficiency of the isolation) and the mixture was incubated for 60 min at 22° C. For basal control measurements, the enzyme was omitted from the reaction mixture. Following incubation SPA beads were added. After 20 min at 22° C. under shaking, the amount of [3H]5′GMP was quantified with a scintillation counter (Topcount, Packard). The results were expressed as a percent inhibition of the control enzyme activity. The standard inhibitory reference compound was zaprinast, which was tested in each experiment at several concentrations to obtain an inhibition curve from which its IC50 value was calculated.
Human phosphodiesterase-3B quantified by measuring the formation of 5′AMP from cAMP using a human recombinant enzyme expressed in Sf9 cells

The test compound (compound 72), reference compound or water (control) were added to a buffer containing 40 mM Tris/HCl (pH 7.4) and 8 mM MgCl2, 450 nM cAMP and 0.25 μCi [3H]cAMP. Thereafter, the reaction was initiated by addition of the enzyme (about 2 U) and the mixture was incubated for 20 min at 22° C. For basal control measurements, the enzyme was omitted from the reaction mixture. Following incubation SPA beads were added. After 30 min at 22° C. under shaking, the amount of [3H]5′AMP was quantified with a scintillation counter (MicroBeta, Perkin Elmer). The results were expressed as a percent inhibition of the control enzyme activity. The standard inhibitory reference compound was milrinone, which was tested in each experiment at several concentrations to obtain an inhibition curve from which its IC50 value was calculated.

Results. As ENT1 inhibitors might potentially be combined with A2A receptor antagonists, it is important not to interfere with A2A receptor signaling (i.e. inhibit cAMP PDEs, which could result in prolongation of A2A receptor signaling). The two phosphodiesterases (PDE) that might be slightly touched at clinically relevant dose of Compound 72 are PDE5 (IC50 1.08 E-06 M) and PDE6 (IC50 3.23 E-06 M). These are cGMP PDEs, therefore treatment with Compound 72 won't interfere with the A2A receptor signaling pathway.

In addition, dipyridamole at clinically relevant concentrations touches PDE11A, which is a cAMP/cGMP dual PDE and could potentially pose a problem in combination with A2A receptor inhibitors. Compound 72 potency for PDE11A was reduced as compared to dipyridamole.

The IC50 has been binned following the ranges: IC50 below 1 μM: +++; IC50 between 1 and 10 μM: ++; IC50 above 10 μM: +. Results are summarized in Table 8.

TABLE 8

| Assay | Compound 72 |
|---|---|
| PDE1B (h) | + |
| PDE2A1 (h) | + |
| PDE3A (h) | + |
| PDE3B (h) | + |
| PDE4A1A (h) | + |
| PDE4B1 (h) | ++ |
| PDE4D2 (h) | ++ |
| PDE5 (h) (non-selective) | ++ |
| PDE6 (non-selective) | ++ |
| PDE7A1 (h) | + |
| PDE8A1 (h) | + |
| PDE10A2 (h) | ++ |
| PDE11A(h) | + |

1. A compound of formula I: 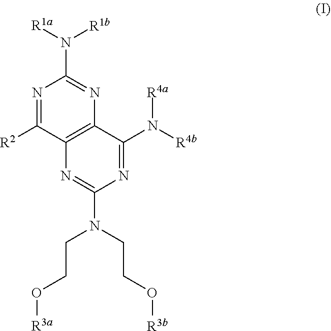
or a pharmaceutically acceptable salt or solvate thereof, wherein
R^{1a} and R^{1b} are each independently selected from: (i) hydrogen, (ii) alkyl optionally substituted with one or more of hydroxy, halo, oxo, amino, —NHS(O)₂NR⁵₂, and alkylsulfonyl, (iii) arylalkyl wherein the aryl part of the arylalkyl is substituted by one or more alkyl, haloalkyl, halogen, alkoxy or —CO2H group, (iv) heteroarylalkyl, (v) heterocyclylalkyl wherein the heterocyclyl part of the heterocyclylalkyl is optionally substituted by one or more alkyl group, (vi) aminocarbonylalkyl, (vii) alkylcarbonylaminoalkyl, (viii) alkyloxyalkyl, (ix) alkylcarbonyloxyalkyl and (x) heterocyclylalkyloxyalkyl; with the condition that R^{1a} and R^{1b} are not both hydrogen;
or R^{1a} and R^{1b} are linked together and form with the nitrogen atom to which they are attached a heterocycle selected from piperidine and piperazine, wherein the heterocycle is optionally substituted with one or more substituent selected from alkyl, alkylcarbonyl, alkyloxycarbonyl, alkylsulfonyl, halo, hydroxy, optionally substituted heteroaryl, and oxo; or the heterocycle is fused with a group selected from aryl and heteroaryl, wherein the aryl or heteroaryl group is optionally substituted with alkyl or alkoxy;
R² represents —NR^{2a}R^{2b} or —OR^{2c}; wherein
R^{2a} and R^{2b} are each independently chosen from: (i) hydrogen, (ii) alkyl, (iii) alkyloxyalkyl, (iv) alkylsulfonylaminoalkyl, (v) arylalkyl wherein the aryl part of the arylalkyl is optionally substituted by one or more of alkoxy, alkylsulfonyl, aminosulfonyl, aminocarbonyl, cyano, halo, haloalkyloxy, optionally substituted heteroaryl, sulfoxide and sulfonylamine groups, (vi) aryloxyalkyl, (vii) cyanoalkyl, (viii) cycloalkyl, (ix) heteroarylalkyl wherein the heteroaryl part of the heteroarylalkyl is optionally substituted by one or more of alkyl, halo, haloalkyl and NH2 groups, (x) heterocyclylalkyl and (xi) hydroxyalkyl;
or R^{2a} and R^{2b} are linked together and form with the nitrogen atom to which they are attached a heterocycle, wherein the heterocycle is optionally substituted with one or more substituent selected from alkoxy, alkyl, alkylcarbonyl, alkylcarbonylamine, alkyloxyalkyl, alkyloxyalkyloxy, alkyloxycarbonyl, alkylsulfonyl, aminocarbonyl, cycloalkyl, cyano, halo, haloalkyl, heteroaryl (optionally substituted by one or more of alkyl, cyano and NH₂ groups), hydroxy, hydroxyalkyl and oxo; or two substituents present on a same carbon atom of the heterocycle are linked together and form a spiro heterocycle; or the heterocycle is fused with a group selected from aryl and heteroaryl, wherein the aryl or heteroaryl group is optionally substituted with alkyl or alkoxy; and
R^{2c} represents is selected from (i) arylalkyl wherein the aryl part of the arylalkyl is optionally substituted by one or more of alkoxy, cyano and halo groups, and (ii) heteroarylalkyl wherein the heteroaryl part of the heteroarylalkyl is optionally substituted by one or more of alkyl and cyano groups;
R^{3a} and R^{3b} are each independently alkyl or alkylcarbonyl;
or R^{3a} is hydrogen and R^{3b} is C₁-C₃ alkyl;
R^{4a} and R^{4b} are linked together and form with the nitrogen atom to which they are attached a heterocycle, wherein the heterocycle optionally comprises one further heteroatom selected from N, S and O; and wherein the heterocycle is optionally substituted with one or more substituent selected from alkoxy, alkyl, haloalkyl, alkyloxycarbonyl, cycloalkyl, halo, heteroaryl optionally substituted by one or more alkyl group, hydroxyl, and oxo; or the heterocycle is fused with a group selected from aryl and heteroaryl, wherein the aryl or heteroaryl group is optionally substituted with alkyl, amine, cyano, or alkoxy; with the condition that when R^{4a} and R^{4b} form a piperidine or a morpholine, then the piperidine or morpholine is substituted by at least one of the listed substituents; and
each R⁵ is independently selected from hydrogen and optionally substituted C₁-C₆ alkyl.

2. The compound according to claim 1, wherein the compound is a compound of formula Ia: 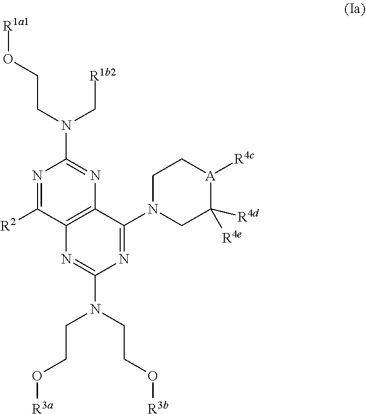
or a pharmaceutically acceptable salt or solvate thereof, wherein
R^{1a1} represents hydrogen, alkyl, alkylcarbonyl or heterocyclylalkyl;
R^{1b2} represents alkylcarbonyloxyalkyl, alkyloxyalkyl, arylalkyl wherein the aryl part of the arylalkyl is substituted by one or more alkoxy group, heterocyclylalkyloxyalkyl or hydroxyalkyl;
A represents CH, N or SO₂;
R^{4c} is absent or represents hydrogen, alkoxy, alkyl, alkyloxycarbonyl, halo or heteroaryl optionally substituted by one or more alkyl groups; with the condition that when A is CH, then R^{4c} is not hydrogen; and
R^{4d} and R^{4e} are both hydrogen atoms or form together an oxo group.

3. The compound according to claim 2, wherein the compound is a compound of formula Ia1: 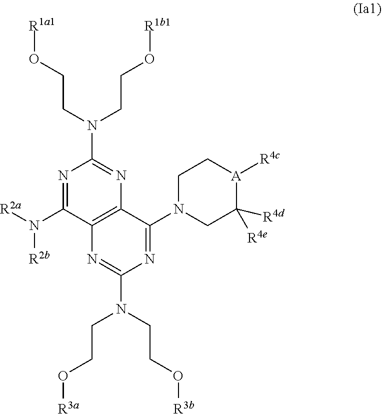
or a pharmaceutically acceptable salt or solvate thereof, wherein
R^{1a1} and R^{1b1} are each independently selected from hydrogen, alkyl, alkylcarbonyl and heterocyclylalkyl.

4. The compound according to claim 3, wherein the compound is selected from:
N2,N2,N6,N6-tetrakis(2-methoxyethyl)-4,8-bis(4-(thiazol-2-yl)piperazin-1-yl)pyrimido[5,4-d]pyrimidine-2,6-diamine;
N2,N2,N6,N6-tetrakis(2-methoxyethyl)-4,8-bis(4-methoxypiperidin-1-yl)pyrimido[5,4-d]pyrimidine-2,6-diamine;
diethyl 1,1′-(2,6-bis(bis(2-methoxyethyl)amino)pyrimido[5,4-d]pyrimidine-4,8-diyl)bis(piperidine-4-carboxylate);
4,8-bis(6,7-dimethoxy-3,4-dihydroisoquinolin-2(1H)-yl)-N2,N2,N6,N6-tetrakis(2-methoxyethyl)pyrimido[5,4-d]pyrimidine-2,6-diamine;
4-(2,6-bis(bis(2-methoxyethyl)amino)-8-(5,6-dihydro-[1,2,4]triazolo[1,5-a]pyrazin-7(8H)-yl)pyrimido[5,4-d]pyrimidin-4-yl)-1-methylpiperazin-2-one;
4-(2,6-bis(bis(2-methoxyethyl)amino)-8-(5,6-dihydro-[1,2,4]triazolo[1,5-a]pyrazin-7(8H)-yl)pyrimido[5,4-d]pyrimidin-4-yl)thiomorpholine 1,1-dioxide;
4-(8-(benzylamino)-2,6-bis(bis(2-methoxyethyl)amino)pyrimido[5,4-d]pyrimidin-4-yl)thiomorpholine 1,1-dioxide;
4-(2,6-bis(bis(2-methoxyethyl)amino)-8-(methyl(propyl)amino)pyrimido[5,4-d]pyrimidin-4-yl)thiomorpholine 1,1-dioxide;
4,8-bis(5,6-dihydro-[1,2,4]triazolo[1,5-a]pyrazin-7(8H)-yl)-N2,N2,N6,N6-tetrakis(2-methoxyethyl)pyrimido[5,4-d]pyrimidine-2,6-diamine;
8-(5,6-dihydro-[1,2,4]triazolo[1,5-a]pyrazin-7(8H)-yl)-N2,N2,N6,N6-tetrakis(2-methoxyethyl)-N4-methyl-N4-propylpyrimido[5,4-d]pyrimidine-2,4,6-triamine;
1,1′-(2,6-bis(bis(2-methoxyethyl)amino)pyrimido[5,4-d]pyrimidine-4,8-diyl)bis(3-(trifluoromethyl)azetidin-3-ol);
1-(8-(benzylamino)-2,6-bis(bis(2-methoxyethyl)amino)pyrimido[5,4-d]pyrimidin-4-yl)-3-(trifluoromethyl)azetidin-3-ol;
1-(2,6-bis(bis(2-methoxyethyl)amino)-8-(methyl(propyl)amino)pyrimido[5,4-d]pyrimidin-4-yl)-3-(trifluoromethyl)azetidin-3-ol;
diethyl 1,1′-(2-(bis(2-hydroxyethyl)amino)-6-(bis(2-methoxyethyl)amino)pyrimido[5,4-d]pyrimidine-4,8-diyl)bis(piperidine-4-carboxylate);
((4,8-bis(4-methoxypiperidin-1-yl)pyrimido[5,4-d]pyrimidine-2,6-diyl)bis(azanetriyl))tetrakis(ethane-2,1-diyl) tetraacetate;
N2,N2,N6-tris(2-methoxyethyl)-4,8-bis(4-methoxypiperidin-1-yl)-N6-(2-(3-(4-methylpiperazin-1-yl)propoxy)ethyl)pyrimido[5,4-d]pyrimidine-2,6-diamine;
2,2′-((6-(bis(2-methoxyethyl)amino)-4,8-bis(4,4-difluoropiperidin-1-yl)pyrimido[5,4-d]pyrimidin-2-yl)azanediyl)diethanol;
2,2′-((6-(bis(2-methoxyethyl)amino)-4,8-bis(4-methoxypiperidin-1-yl)pyrimido[5,4-d]pyrimidin-2-yl)azanediyl)diethanol;
N,N-bis(2-methoxyethyl)-4,8-bis(4-methoxypiperidin-1-yl)-6-(4-(methylsulfonyl)piperazin-1-yl)pyrimido[5,4-d]pyrimidin-2-amine;
ethyl 4-(6-(bis(2-methoxyethyl)amino)-4,8-bis(4-methoxypiperidin-1-yl)pyrimido[5,4-d]pyrimidin-2-yl)piperazine-1-carboxylate;
N2-(2-(1H-pyrazol-1-yl)ethyl)-N6,N6-bis(2-methoxyethyl)-4,8-bis(4-methoxypiperidin-1-yl)pyrimido[5,4-d]pyrimidine-2,6-diamine;
4,8-bis(3,3-difluoropiperidin-1-yl)-N2,N2,N6,N6-tetrakis(2-methoxyethyl)pyrimido[5,4-d]pyrimidine-2,6-diamine;
2-((6-(bis(2-methoxyethyl)amino)-4,8-bis(4-methoxypiperidin-1-yl)pyrimido[5,4-d]pyrimidin-2-yl)(2-methoxyethyl)amino)ethanol;
4,4′-(2,6-bis(bis(2-methoxyethyl)amino)pyrimido[5,4-d]pyrimidine-4,8-diyl)bis(thiomorpholine 1,1-dioxide);
2,2′-((6-(bis(2-methoxyethyl)amino)-4-(piperidin-1-yl)-8-(4-(thiazol-2-yl)piperazin-1-yl)pyrimido[5,4-d]pyrimidin-2-yl)azanediyl)diethanol;
2,2′-((6-(bis(2-methoxyethyl)amino)-8-(piperidin-1-yl)-4-(4-(thiazol-2-yl)piperazin-1-yl)pyrimido[5,4-d]pyrimidin-2-yl)azanediyl)diethanol;
N2,N2,N6-tris(2-methoxyethyl)-4,8-bis(4-methoxypiperidin-1-yl)-N6-(2-(3-morpholinopropoxy)ethyl)pyrimido[5,4-d]pyrimidine-2,6-diamine;
N2,N2,N6-tris(2-methoxyethyl)-4,8-bis(4-methoxypiperidin-1-yl)-N6-(2-(2-morpholinoethoxy)ethyl)pyrimido[5,4-d]pyrimidine-2,6-diamine;
N2,N2,N6,N6-tetrakis(2-methoxyethyl)-8-(4-methoxypiperidin-1-yl)-N4-((tetrahydro-2H-pyran-4-yl)methyl)pyrimido[5,4-d]pyrimidine-2,4,6-triamine;
N4-(4-methoxybenzyl)-N2,N2,N6,N6-tetrakis(2-methoxyethyl)-8-(4-methoxypiperidin-1-yl)pyrimido[5,4-d]pyrimidine-2,4,6-triamine;
N4-(3-methoxybenzyl)-N2,N2,N6,N6-tetrakis(2-methoxyethyl)-8-(4-methoxypiperidin-1-yl)pyrimido[5,4-d]pyrimidine-2,4,6-triamine;
N4-benzyl-N2,N2,N6,N6-tetrakis(2-methoxyethyl)-8-(4-methoxypiperidin-1-yl)pyrimido[5,4-d]pyrimidine-2,4,6-triamine;
N2,N2,N6,N6-tetrakis(2-methoxyethyl)-8-(4-methoxypiperidin-1-yl)-N4-neopentylpyrimido[5,4-d]pyrimidine-2,4,6-triamine;
N2,N2,N6,N6-tetrakis(2-methoxyethyl)-8-(4-methoxypiperidin-1-yl)-N4-(3-methoxypropyl)pyrimido[5,4-d]pyrimidine-2,4,6-triamine;
N4-(3-ethoxypropyl)-N2,N2,N6,N6-tetrakis(2-methoxyethyl)-8-(4-methoxypiperidin-1-yl)pyrimido[5,4-d]pyrimidine-2,4,6-triamine;
N2,N2,N6,N6-tetrakis(2-methoxyethyl)-8-(4-methoxypiperidin-1-yl)-N4-((6-(trifluoromethyl)pyridin-3-yl)methyl)pyrimido[5,4-d]pyrimidine-2,4,6-triamine;
N4-butyl-N2,N2,N6,N6-tetrakis(2-methoxyethyl)-8-(4-methoxypiperidin-1-yl)pyrimido[5,4-d]pyrimidine-2,4,6-triamine;
N4-(cyclopentylmethyl)-N2,N2,N6,N6-tetrakis(2-methoxyethyl)-8-(4-methoxypiperidin-1-yl)pyrimido[5,4-d]pyrimidine-2,4,6-triamine;
N2,N2,N6,N6-tetrakis(2-methoxyethyl)-8-(4-methoxypiperidin-1-yl)-N4-(2-phenoxyethyl)pyrimido[5,4-d]pyrimidine-2,4,6-triamine;
N2,N2,N6,N6-tetrakis(2-methoxyethyl)-8-(4-methoxypiperidin-1-yl)-N4-((tetrahydrofuran-3-yl)methyl)pyrimido[5,4-d]pyrimidine-2,4,6-triamine N2,N2,N6,N6-tetrakis(2-methoxyethyl)-8-(4-methoxypiperidin-1-yl)-N4-(thiazol-2-ylmethyl)pyrimido[5,4-d]pyrimidine-2,4,6-triamine;
N2,N2,N6,N6-tetrakis(2-methoxyethyl)-8-(4-methoxypiperidin-1-yl)-N4-(thiazol-5-ylmethyl)pyrimido[5,4-d]pyrimidine-2,4,6-triamine;
N2,N2,N6,N6-tetrakis(2-methoxyethyl)-8-(4-methoxypiperidin-1-yl)-N4-(oxazol-5-ylmethyl)pyrimido[5,4-d]pyrimidine-2,4,6-triamine;
N2,N2,N6,N6-tetrakis(2-methoxyethyl)-8-(4-methoxypiperidin-1-yl)-N4-(pyridin-4-ylmethyl)pyrimido[5,4-d]pyrimidine-2,4,6-triamine;
N2,N2,N6,N6-tetrakis(2-methoxyethyl)-8-(4-methoxypiperidin-1-yl)-N4-(pyridin-3-ylmethyl)pyrimido[5,4-d]pyrimidine-2,4,6-triamine;
N2,N2,N6,N6-tetrakis(2-methoxyethyl)-8-(4-methoxypiperidin-1-yl)-N4-(2-(pyridin-3-yl)ethyl)pyrimido[5,4-d]pyrimidine-2,4,6-triamine;
N2,N2,N6,N6-tetrakis(2-methoxyethyl)-8-(4-methoxypiperidin-1-yl)-N4-(2-(pyridin-4-yl)ethyl)pyrimido[5,4-d]pyrimidine-2,4,6-triamine;
N2,N2,N6,N6-tetrakis(2-methoxyethyl)-8-(4-methoxypiperidin-1-yl)-N4-((1-methyl-1H-pyrazol-4-yl)methyl)pyrimido[5,4-d]pyrimidine-2,4,6-triamine;
N2,N2,N6,N6-tetrakis(2-methoxyethyl)-8-(4-methoxypiperidin-1-yl)-N4-(thiazol-4-ylmethyl)pyrimido[5,4-d]pyrimidine-2,4,6-triamine;
N2,N2,N6,N6-tetrakis(2-methoxyethyl)-8-(4-methoxypiperidin-1-yl)-N4,N4-dimethylpyrimido[5,4-d]pyrimidine-2,4,6-triamine;
N2,N2,N6,N6-tetrakis(2-methoxyethyl)-8-(4-methoxypiperidin-1-yl)-N4-methyl-N4-propylpyrimido[5,4-d]pyrimidine-2,4,6-triamine;
N2,N2,N6,N6-tetrakis(2-methoxyethyl)-4-(4-methoxypiperidin-1-yl)-8-morpholinopyrimido[5,4-d]pyrimidine-2,6-diamine;
4-(2,6-bis(bis(2-methoxyethyl)amino)-8-(4-methoxypiperidin-1-yl)pyrimido[5,4-d]pyrimidin-4-yl)piperazin-2-one;
1-(2,6-bis(bis(2-methoxyethyl)amino)-8-(4-methoxypiperidin-1-yl)pyrimido[5,4-d]pyrimidin-4-yl)piperidin-4-ol;
4-(2,6-bis(bis(2-methoxyethyl)amino)-8-(4-methoxypiperidin-1-yl)pyrimido[5,4-d]pyrimidin-4-yl)-1-methylpiperazin-2-one;
N2,N2,N6,N6-tetrakis(2-methoxyethyl)-8-(4-methoxypiperidin-1-yl)-N4-methyl-N4-(pyridin-2-ylmethyl)pyrimido[5,4-d]pyrimidine-2,4,6-triamine;
1-(4-(2,6-bis(bis(2-methoxyethyl)amino)-8-(4-methoxypiperidin-1-yl)pyrimido[5,4-d]pyrimidin-4-yl)piperazin-1-yl)ethenone;
1-(2,6-bis(bis(2-methoxyethyl)amino)-8-(4-methoxypiperidin-1-yl)pyrimido[5,4-d]pyrimidin-4-yl)piperidine-4-carboxamide;
N2,N2,N6,N6-tetrakis(2-methoxyethyl)-4-(4-(methoxymethyl)piperidin-1-yl)-8-(4-methoxypiperidin-1-yl)pyrimido[5,4-d]pyrimidine-2,6-diamine;
4-(2,6-bis(bis(2-methoxyethyl)amino)-8-(4-methoxypiperidin-1-yl)pyrimido[5,4-d]pyrimidin-4-yl)thiomorpholine 1,1-dioxide;
N2,N2,N6,N6-tetrakis(2-methoxyethyl)-4-(4-methoxypiperidin-1-yl)-8-(4-(methylsulfonyl)piperidin-1-yl)pyrimido[5,4-d]pyrimidine-2,6-diamine;
N2,N2,N6,N6-tetrakis(2-methoxyethyl)-4-(4-methoxypiperidin-1-yl)-8-(1,4-oxazepan-4-yl)pyrimido[5,4-d]pyrimidine-2,6-diamine;
N2,N2,N6,N6-tetrakis(2-methoxyethyl)-4-(4-methoxypiperidin-1-yl)-8-(3-methoxypyrrolidin-1-yl)pyrimido[5,4-d]pyrimidine-2,6-diamine;
N2,N2,N6,N6-tetrakis(2-methoxyethyl)-8-(4-methoxypiperidin-1-yl)-N4-methyl-N4-(pyridin-3-ylmethyl)pyrimido[5,4-d]pyrimidine-2,4,6-triamine;
N2,N2,N6,N6-tetrakis(2-methoxyethyl)-8-(4-methoxypiperidin-1-yl)-N4-methyl-N4-(pyridin-4-ylmethyl)pyrimido[5,4-d]pyrimidine-2,4,6-triamine;
N2,N2,N6,N6-tetrakis(2-methoxyethyl)-8-(4-methoxypiperidin-1-yl)-N4-methyl-N4-(2-(pyridin-2-yl)ethyl)pyrimido[5,4-d]pyrimidine-2,4,6-triamine;
N2,N2,N6,N6-tetrakis(2-methoxyethyl)-8-(4-methoxypiperidin-1-yl)-N4-(pyrazin-2-ylmethyl)pyrimido[5,4-d]pyrimidine-2,4,6-triamine;
N4,N4-diethyl-N2,N2,N6,N6-tetrakis(2-methoxyethyl)-8-(4-methoxypiperidin-1-yl)pyrimido[5,4-d]pyrimidine-2,4,6-triamine;
4-(3-methoxyazetidin-1-yl)-N2,N2,N6,N6-tetrakis(2-methoxyethyl)-8-(4-methoxypiperidin-1-yl)pyrimido[5,4-d]pyrimidine-2,6-diamine;
N2,N2,N6,N6-tetrakis(2-methoxyethyl)-4-(4-methoxypiperidin-1-yl)-8-(2-methylmorpholino)pyrimido[5,4-d]pyrimidine-2,6-diamine;
N-(1-(2,6-bis(bis(2-methoxyethyl)amino)-8-(4-methoxypiperidin-1-yl)pyrimido[5,4-d]pyrimidin-4-yl)piperidin-4-yl)acetamide;
N2,N2,N6,N6-tetrakis(2-methoxyethyl)-4-(4-methoxypiperidin-1-yl)-8-(pyrrolidin-1-yl)pyrimido[5,4-d]pyrimidine-2,6-diamine;
1-(2,6-bis(bis(2-methoxyethyl)amino)-8-(4-methoxypiperidin-1-yl)pyrimido[5,4-d]pyrimidin-4-yl)piperidine-4-carbonitrile;
N2,N2,N6,N6-tetrakis(2-methoxyethyl)-4-(4-methoxypiperidin-1-yl)-8-(3-methylpyrrolidin-1-yl)pyrimido[5,4-d]pyrimidine-2,6-diamine;
(1-(2,6-bis(bis(2-methoxyethyl)amino)-8-(4-methoxypiperidin-1-yl)pyrimido[5,4-d]pyrimidin-4-yl)azetidin-3-yl)methanol;
(R)-1-(2,6-bis(bis(2-methoxyethyl)amino)-8-(4-methoxypiperidin-1-yl)pyrimido[5,4-d]pyrimidin-4-yl)pyrrolidin-3-ol;
1-(2,6-bis(bis(2-methoxyethyl)amino)-8-(4-methoxypiperidin-1-yl)pyrimido[5,4-d]pyrimidin-4-yl)pyrrolidin-3-ol;
N4-(2-(1H-imidazol-5-yl)ethyl)-N2,N2,N6,N6-tetrakis(2-methoxyethyl)-8-(4-methoxypiperidin-1-yl)pyrimido[5,4-d]pyrimidine-2,4,6-triamine;
N2,N2,N6,N6-tetrakis(2-methoxyethyl)-4-(3-methoxypiperidin-1-yl)-8-(4-methoxypiperidin-1-yl)pyrimido[5,4-d]pyrimidine-2,6-diamine;
N2,N2,N6,N6-tetrakis(2-methoxyethyl)-4-(4-methoxypiperidin-1-yl)-8-(2-oxa-6-azaspiro[3.5]nonan-6-yl)pyrimido[5,4-d]pyrimidine-2,6-diamine;
1-(4-(2,6-bis(bis(2-methoxyethyl)amino)-8-(4-methoxypiperidin-1-yl)pyrimido[5,4-d]pyrimidin-4-yl)-1,4-diazepan-1-yl)ethenone;
4-(3-fluoroazetidin-1-yl)-N2,N2,N6,N6-tetrakis(2-methoxyethyl)-8-(4-methoxypiperidin-1-yl)pyrimido[5,4-d]pyrimidine-2,6-diamine;
4-(3,3-dimethylazetidin-1-yl)-N2,N2,N6,N6-tetrakis(2-methoxyethyl)-8-(4-methoxypiperidin-1-yl)pyrimido[5,4-d]pyrimidine-2,6-diamine;
1-(2,6-bis(bis(2-methoxyethyl)amino)-8-(4-methoxypiperidin-1-yl)pyrimido[5,4-d]pyrimidin-4-yl)-3-methylazetidin-3-ol;
(S)-4-(3-fluoropyrrolidin-1-yl)-N2,N2,N6,N6-tetrakis(2-methoxyethyl)-8-(4-methoxypiperidin-1-yl)pyrimido[5,4-d]pyrimidine-2,6-diamine;
(R)-4-(3-fluoropyrrolidin-1-yl)-N2,N2,N6,N6-tetrakis(2-methoxyethyl)-8-(4-methoxypiperidin-1-yl)pyrimido[5,4-d]pyrimidine-2,6-diamine;
4-(3-fluoropyrrolidin-1-yl)-N2,N2,N6,N6-tetrakis(2-methoxyethyl)-8-(4-methoxypiperidin-1-yl)pyrimido[5,4-d]pyrimidine-2,6-diamine;
4-(3,3-difluoroazetidin-1-yl)-N2,N2,N6,N6-tetrakis(2-methoxyethyl)-8-(4-methoxypiperidin-1-yl)pyrimido[5,4-d]pyrimidine-2,6-diamine;
(S)—N2,N2,N6,N6-tetrakis(2-methoxyethyl)-4-(4-methoxypiperidin-1-yl)-8-(3-methoxypyrrolidin-1-yl)pyrimido[5,4-d]pyrimidine-2,6-diamine;
(R)—N2,N2,N6,N6-tetrakis(2-methoxyethyl)-4-(4-methoxypiperidin-1-yl)-8-(3-methoxypyrrolidin-1-yl)pyrimido[5,4-d]pyrimidine-2,6-diamine;
4-(4-fluoropiperidin-1-yl)-N2,N2,N6,N6-tetrakis(2-methoxyethyl)-8-(4-methoxypiperidin-1-yl)pyrimido[5,4-d]pyrimidine-2,6-diamine;
4-(3,3-difluoropyrrolidin-1-yl)-N2,N2,N6,N6-tetrakis(2-methoxyethyl)-8-(4-methoxypiperidin-1-yl)pyrimido[5,4-d]pyrimidine-2,6-diamine;
4-((2R,6S)-2,6-dimethylmorpholino)-N2,N2,N6,N6-tetrakis(2-methoxyethyl)-8-(4-methoxypiperidin-1-yl)pyrimido[5,4-d]pyrimidine-2,6-diamine;
1-(2,6-bis(bis(2-methoxyethyl)amino)-8-(4-methoxypiperidin-1-yl)pyrimido[5,4-d]pyrimidin-4-yl)-3-(trifluoromethyl)azetidin-3-ol;
2-((2,6-bis(bis(2-methoxyethyl)amino)-8-(4-methoxypiperidin-1-yl)pyrimido[5,4-d]pyrimidin-4-yl)(methyl)amino)ethanol;
N2,N2,N6,N6-tetrakis(2-methoxyethyl)-4-(4-methoxypiperidin-1-yl)-8-(2-methylmorpholino)pyrimido[5,4-d]pyrimidine-2,6-diamine;
4-(5,6-dihydroimidazo[1,5-a]pyrazin-7(8H)-yl)-N2,N2,N6,N6-tetrakis(2-methoxyethyl)-8-(4-methoxypiperidin-1-yl)pyrimido[5,4-d]pyrimidine-2,6-diamine;
(1-(2,6-bis(bis(2-methoxyethyl)amino)-8-(4-methoxypiperidin-1-yl)pyrimido[5,4-d]pyrimidin-4-yl)piperidin-3-yl)methanol;
4-(5,6-dihydro-[1,2,4]triazolo[1,5-a]pyrazin-7(8H)-yl)-N2,N2,N6,N6-tetrakis(2-methoxyethyl)-8-(4-methoxypiperidin-1-yl)pyrimido[5,4-d]pyrimidine-2,6-diamine;
8-(2,6-bis(bis(2-methoxyethyl)amino)-8-(4-methoxypiperidin-1-yl)pyrimido[5,4-d]pyrimidin-4-yl)-1,3,8-triazaspiro[4.5]decane-2,4-dione;
N2,N2,N6,N6-tetrakis(2-methoxyethyl)-4-(4-methoxypiperidin-1-yl)-8-(4-(pyridin-3-yl)piperazin-1-yl)pyrimido[5,4-d]pyrimidine-2,6-diamine;
N2,N2,N6,N6-tetrakis(2-methoxyethyl)-4-(4-methoxypiperidin-1-yl)-8-(4-(pyrimidin-2-yl)piperazin-1-yl)pyrimido[5,4-d]pyrimidine-2,6-diamine;
N2,N2,N6,N6-tetrakis(2-methoxyethyl)-4-(4-methoxypiperidin-1-yl)-8-(4-(pyrazin-2-yl)piperazin-1-yl)pyrimido[5,4-d]pyrimidine-2,6-diamine;
4-(4-(3-aminopyridin-2-yl)piperazin-1-yl)-N2,N2,N6,N6-tetrakis(2-methoxyethyl)-8-(4-methoxypiperidin-1-yl)pyrimido[5,4-d]pyrimidine-2,6-diamine;
N2,N2,N6,N6-tetrakis(2-methoxyethyl)-4-(4-methoxypiperidin-1-yl)-8-(4-(4-methylpyrimidin-2-yl)piperazin-1-yl)pyrimido[5,4-d]pyrimidine-2,6-diamine;
N2,N2,N6,N6-tetrakis(2-methoxyethyl)-4-(4-methoxypiperidin-1-yl)-8-(4-(3-methyl-1,2,4-thiadiazol-5-yl)piperazin-1-yl)pyrimido[5,4-d]pyrimidine-2,6-diamine;
6-(4-(2,6-bis(bis(2-methoxyethyl)amino)-8-(4-methoxypiperidin-1-yl)pyrimido[5,4-d]pyrimidin-4-yl)piperazin-1-yl)nicotinonitrile;
N2,N2,N6,N6-tetrakis(2-methoxyethyl)-4-(4-methoxypiperidin-1-yl)-8-(4-(4-methoxypyrimidin-2-yl)piperazin-1-yl)pyrimido[5,4-d]pyrimidine-2,6-diamine;
N4-ethyl-N2,N2,N6,N6-tetrakis(2-methoxyethyl)-8-(4-methoxypiperidin-1-yl)pyrimido[5,4-d]pyrimidine-2,4,6-triamine;
N4-isobutyl-N2,N2,N6,N6-tetrakis(2-methoxyethyl)-8-(4-methoxypiperidin-1-yl)pyrimido[5,4-d]pyrimidine-2,4,6-triamine;
N2,N2,N4,N6,N6-pentakis(2-methoxyethyl)-8-(4-methoxypiperidin-1-yl)pyrimido[5,4-d]pyrimidine-2,4,6-triamine;
N2,N2,N6,N6-tetrakis(2-methoxyethyl)-4,8-bis(4-(3-methyl-1,2,4-oxadiazol-5-yl)piperazin-1-yl)pyrimido[5,4-d]pyrimidine-2,6-diamine;
N2,N2,N6,N6-tetrakis(2-methoxyethyl)-4,8-bis(4-(5-methyl-1,3,4-oxadiazol-2-yl)piperazin-1-yl)pyrimido[5,4-d]pyrimidine-2,6-diamine;
N2,N2,N6,N6-tetrakis(2-methoxyethyl)-4,8-bis(4-(1-methyl-1H-1,2,4-triazol-3-yl)piperazin-1-yl)pyrimido[5,4-d]pyrimidine-2,6-diamine;
N2-ethyl-N6,N6-bis(2-methoxyethyl)-4,8-bis(4-methoxypiperidin-1-yl)-N2-(2-(pyrrolidin-1-yl)ethyl)pyrimido[5,4-d]pyrimidine-2,6-diamine;
N2-ethyl-N6,N6-bis(2-methoxyethyl)-4,8-bis(4-methoxypiperidin-1-yl)-N2-(2-(piperidin-1-yl)ethyl)pyrimido[5,4-d]pyrimidine-2,6-diamine;
N2-(3-(1H-imidazol-1-yl)propyl)-N6,N6-bis(2-methoxyethyl)-4,8-bis(4-methoxypiperidin-1-yl)pyrimido[5,4-d]pyrimidine-2,6-diamine;
N2,N2,N6,N6-tetrakis(2-methoxyethyl)-4-(4-methoxypiperidin-1-yl)-8-(4-(5-methyl-1,3,4-oxadiazol-2-yl)piperazin-1-yl)pyrimido[5,4-d]pyrimidine-2,6-diamine;
(1-(2,6-bis(bis(2-methoxyethyl)amino)-8-(4-methoxypiperidin-1-yl)pyrimido[5,4-d]pyrimidin-4-yl)pyrrolidin-3-yl)methanol;
N4-(3,4-dichlorobenzyl)-N2,N2,N6,N6-tetrakis(2-methoxyethyl)-8-(4-methoxypiperidin-1-yl)pyrimido[5,4-d]pyrimidine-2,4,6-triamine;
N4-(3-chloro-4-fluorobenzyl)-N2,N2,N6,N6-tetrakis(2-methoxyethyl)-8-(4-methoxypiperidin-1-yl)pyrimido[5,4-d]pyrimidine-2,4,6-triamine;
N4-(3,4-difluorobenzyl)-N2,N2,N6,N6-tetrakis(2-methoxyethyl)-8-(4-methoxypiperidin-1-yl)pyrimido[5,4-d]pyrimidine-2,4,6-triamine;
4-(((2,6-bis(bis(2-methoxyethyl)amino)-8-(4-methoxypiperidin-1-yl)pyrimido[5,4-d]pyrimidin-4-yl)amino)methyl)benzonitrile;
N4-(3-chlorobenzyl)-N2,N2,N6,N6-tetrakis(2-methoxyethyl)-8-(4-methoxypiperidin-1-yl)pyrimido[5,4-d]pyrimidine-2,4,6-triamine;
N4-(3-fluorobenzyl)-N2,N2,N6,N6-tetrakis(2-methoxyethyl)-8-(4-methoxypiperidin-1-yl)pyrimido[5,4-d]pyrimidine-2,4,6-triamine;
N2,N2,N6,N6-tetrakis(2-methoxyethyl)-8-(4-methoxypiperidin-1-yl)-N4-(3-(trifluoromethoxy)benzyl)pyrimido[5,4-d]pyrimidine-2,4,6-triamine;
N4-(3-(difluoromethoxy)benzyl)-N2,N2,N6,N6-tetrakis(2-methoxyethyl)-8-(4-methoxypiperidin-1-yl)pyrimido[5,4-d]pyrimidine-2,4,6-triamine;
3-(((2,6-bis(bis(2-methoxyethyl)amino)-8-(4-methoxypiperidin-1-yl)pyrimido[5,4-d]pyrimidin-4-yl)amino)methyl)benzonitrile;
N4-(3-(1H-pyrazol-1-yl)benzyl)-N2,N2,N6,N6-tetrakis(2-methoxyethyl)-8-(4-methoxypiperidin-1-yl)pyrimido[5,4-d]pyrimidine-2,4,6-triamine;
N4-((2-aminopyridin-4-yl)methyl)-N2,N2,N6,N6-tetrakis(2-methoxyethyl)-8-(4-methoxypiperidin-1-yl)pyrimido[5,4-d]pyrimidine-2,4,6-triamine;
N2,N2,N6,N6-tetrakis(2-methoxyethyl)-8-(4-methoxypiperidin-1-yl)-N4-(3-(methylsulfonyl)benzyl)pyrimido[5,4-d]pyrimidine-2,4,6-triamine;
5-(((2,6-bis(bis(2-methoxyethyl)amino)-8-(4-methoxypiperidin-1-yl)pyrimido[5,4-d]pyrimidin-4-yl)amino)methyl)-2-fluorobenzonitrile;
5-(((2,6-bis(bis(2-methoxyethyl)amino)-8-(4-methoxypiperidin-1-yl)pyrimido[5,4-d]pyrimidin-4-yl)amino)methyl)-2-chlorobenzonitrile;
N2,N2,N6,N6-tetrakis(2-methoxyethyl)-8-(4-methoxypiperidin-1-yl)-N4-(3-(oxazol-2-yl)benzyl)pyrimido[5,4-d]pyrimidine-2,4,6-triamine;
4-((3,5-dimethoxybenzyl)oxy)-N2,N2,N6,N6-tetrakis(2-methoxyethyl)-8-(4-methoxypiperidin-1-yl)pyrimido[5,4-d]pyrimidine-2,6-diamine;
4-((3-methoxybenzyl)oxy)-N2,N2,N6,N6-tetrakis(2-methoxyethyl)-8-(4-methoxypiperidin-1-yl)pyrimido[5,4-d]pyrimidine-2,6-diamine;
4-((3,4-dichlorobenzyl)oxy)-N2,N2,N6,N6-tetrakis(2-methoxyethyl)-8-(4-methoxypiperidin-1-yl)pyrimido[5,4-d]pyrimidine-2,6-diamine;
4-((3-chlorobenzyl)oxy)-N2,N2,N6,N6-tetrakis(2-methoxyethyl)-8-(4-methoxypiperidin-1-yl)pyrimido[5,4-d]pyrimidine-2,6-diamine;
3-(((2,6-bis(bis(2-methoxyethyl)amino)-8-(4-methoxypiperidin-1-yl)pyrimido[5,4-d]pyrimidin-4-yl)oxy)methyl)benzonitrile;
4-((4-fluoro-3-methoxybenzyl)oxy)-N2,N2,N6,N6-tetrakis(2-methoxyethyl)-8-(4-methoxypiperidin-1-yl)pyrimido[5,4-d]pyrimidine-2,6-diamine;
5-(((2,6-bis(bis(2-methoxyethyl)amino)-8-(4-methoxypiperidin-1-yl)pyrimido[5,4-d]pyrimidin-4-yl)oxy)methyl)nicotinonitrile;
N2,N2,N6,N6-tetrakis(2-methoxyethyl)-4-(4-methoxypiperidin-1-yl)-8-((3-(5-methyl-1,2,4-oxadiazol-3-yl)benzyl)oxy)pyrimido[5,4-d]pyrimidine-2,6-diamine;
N4-((2-chlorothiazol-5-yl)methyl)-N2,N2,N6,N6-tetrakis(2-methoxyethyl)-8-(4-methoxypiperidin-1-yl)pyrimido[5,4-d]pyrimidine-2,4,6-triamine;
N2,N2,N6,N6-tetrakis(2-methoxyethyl)-8-(4-methoxypiperidin-1-yl)-N4-((5-methylfuran-2-yl)methyl)pyrimido[5,4-d]pyrimidine-2,4,6-triamine;
N2,N2,N6,N6-tetrakis(2-methoxyethyl)-8-(4-methoxypiperidin-1-yl)-N4-((3-methylisoxazol-5-yl)methyl)pyrimido[5,4-d]pyrimidine-2,4,6-triamine;
N4-((1-isopropyl-1H-pyrazol-4-yl)methyl)-N2,N2,N6,N6-tetrakis(2-methoxyethyl)-8-(4-methoxypiperidin-1-yl)pyrimido[5,4-d]pyrimidine-2,4,6-triamine;
4-((1-isopropyl-1H-pyrazol-4-yl)methoxy)-N2,N2,N6,N6-tetrakis(2-methoxyethyl)-8-(4-methoxypiperidin-1-yl)pyrimido[5,4-d]pyrimidine-2,6-diamine;
N2,N2,N6,N6-tetrakis(2-methoxyethyl)-8-(4-methoxypiperidin-1-yl)-N4,N4-dipropylpyrimido[5,4-d]pyrimidine-2,4,6-triamine;
N4-cyclopentyl-N2,N2,N6,N6-tetrakis(2-methoxyethyl)-8-(4-methoxypiperidin-1-yl)-N4-methylpyrimido[5,4-d]pyrimidine-2,4,6-triamine;
N4-cyclopentyl-N2,N2,N6,N6-tetrakis(2-methoxyethyl)-8-(4-methoxypiperidin-1-yl)pyrimido[5,4-d]pyrimidine-2,4,6-triamine;
N4-isobutyl-N2,N2,N6,N6-tetrakis(2-methoxyethyl)-8-(4-methoxypiperidin-1-yl)-N4-methylpyrimido[5,4-d]pyrimidine-2,4,6-triamine;
N4-(3,4-dimethoxybenzyl)-N2,N2,N6,N6-tetrakis(2-methoxyethyl)-8-(4-methoxypiperidin-1-yl)-N4-methylpyrimido[5,4-d]pyrimidine-2,4,6-triamine;
N4-(3,4-dimethoxybenzyl)-N2,N2,N6,N6-tetrakis(2-methoxyethyl)-8-(4-methoxypiperidin-1-yl)pyrimido[5,4-d]pyrimidine-2,4,6-triamine;
N4-(3-methoxybenzyl)-N2,N2,N6,N6-tetrakis(2-methoxyethyl)-8-(4-methoxypiperidin-1-yl)-N4-methylpyrimido[5,4-d]pyrimidine-2,4,6-triamine;
2-(2-(2-((6-(bis(2-methoxyethyl)amino)-4,8-bis(4-methoxypiperidin-1-yl)pyrimido[5,4-d]pyrimidin-2-yl)(2-methoxyethyl)amino)ethoxy)ethyl)isoindoline-1,3-dione;
2,2′-((6-(bis(2-methoxyethyl)amino)-4,8-bis(4-(thiazol-2-yl)piperazin-1-yl)pyrimido[5,4-d]pyrimidin-2-yl)azanediyl)diethanol;
N2,N2,N6,N6-tetrakis(2-methoxyethyl)-4-(piperidin-1-yl)-8-(4-(thiazol-2-yl)piperazin-1-yl)pyrimido[5,4-d]pyrimidine-2,6-diamine;
3-((2,6-bis(bis(2-methoxyethyl)amino)-8-(4-methoxypiperidin-1-yl)pyrimido[5,4-d]pyrimidin-4-yl)amino)propan-1-ol;
N4-(3-(1H-imidazol-1-yl)propyl)-N2,N2,N6,N6-tetrakis(2-methoxyethyl)-8-(4-methoxypiperidin-1-yl)pyrimido[5,4-d]pyrimidine-2,4,6-triamine;
N2,N2,N6,N6-tetrakis(2-methoxyethyl)-8-(4-methoxypiperidin-1-yl)-N4-(3-(pyridin-2-yl)propyl)pyrimido[5,4-d]pyrimidine-2,4,6-triamine;
4-((2,6-bis(bis(2-methoxyethyl)amino)-8-(4-methoxypiperidin-1-yl)pyrimido[5,4-d]pyrimidin-4-yl)amino)-2-methylbutan-2-ol;
5-((2,6-bis(bis(2-methoxyethyl)amino)-8-(4-methoxypiperidin-1-yl)pyrimido[5,4-d]pyrimidin-4-yl)amino)pentanenitrile;
N-(3-((2,6-bis(bis(2-methoxyethyl)amino)-8-(4-methoxypiperidin-1-yl)pyrimido[5,4-d]pyrimidin-4-yl)amino)propyl)methanesulfonamide;
N4-(4-methoxybutyl)-N2,N2,N6,N6-tetrakis(2-methoxyethyl)-8-(4-methoxypiperidin-1-yl)pyrimido[5,4-d]pyrimidine-2,4,6-triamine;
6-((2,6-bis(bis(2-methoxyethyl)amino)-8-(4-methoxypiperidin-1-yl)pyrimido[5,4-d]pyrimidin-4-yl)amino)hexanenitrile;
N2,N2,N6-tris(2-methoxyethyl)-4,8-bis(4-methoxypiperidin-1-yl)-N6-(2,3,4-trimethoxybenzyl)pyrimido[5,4-d]pyrimidine-2,6-diamine;
N2,N2,N6-tris(2-methoxyethyl)-4,8-bis(4-methoxypiperidin-1-yl)-N6-(3,4,5-trimethoxybenzyl)pyrimido[5,4-d]pyrimidine-2,6-diamine;
2-((6-(bis(2-methoxyethyl)amino)-4,8-bis(4-methoxypiperidin-1-yl)pyrimido[5,4-d]pyrimi<din-2-yl)(2,3,4-trimethoxybenzyl)amino)ethanol;
2-((6-(bis(2-methoxyethyl)amino)-4,8-bis(4-methoxypiperidin-1-yl)pyrimido[5,4-d]pyrimidin-2-yl)(3,4,5-trimethoxybenzyl)amino)ethanol;
4,4′-(2,6-bis(bis(2-methoxyethyl)amino)pyrimido[5,4-d]pyrimidine-4,8-diyl)bis(1-methylpiperazin-2-one);
4-(2,6-bis(bis(2-methoxyethyl)amino)-8-(1,1-dioxidothiomorpholino)pyrimido[5,4-d]pyrimidin-4-yl)-1-methylpiperazin-2-one;
4-(2,6-bis(bis(2-methoxyethyl)amino)-8-(3-hydroxy-3-(trifluoromethyl)azetidin-1-yl)pyrimido[5,4-d]pyrimidin-4-yl)-1-methylpiperazin-2-one;
4-(8-(benzylamino)-2,6-bis(bis(2-methoxyethyl)amino)pyrimido[5,4-d]pyrimidin-4-yl)-1-methylpiperazin-2-one;
4-(2,6-bis(bis(2-methoxyethyl)amino)-8-(methyl(propyl)amino)pyrimido[5,4-d]pyrimidin-4-yl)-1-methylpiperazin-2-one;
4-(2,6-bis(bis(2-methoxyethyl)amino)-8-(3-hydroxy-3-(trifluoromethyl)azetidin-1-yl)pyrimido[5,4-d]pyrimidin-4-yl)thiomorpholine 1,1-dioxide;
1-(2,6-bis(bis(2-methoxyethyl)amino)-8-(5,6-dihydro-[1,2,4]triazolo[1,5-a]pyrazin-7(8H)-yl)pyrimido[5,4-d]pyrimidin-4-yl)-3-(trifluoromethyl)azetidin-3-ol;
N4-benzyl-8-(5,6-dihydro-[1,2,4]triazolo[1,5-a]pyrazin-7(8H)-yl)-N2,N2,N6,N6-tetrakis(2-methoxyethyl)pyrimido[5,4-d]pyrimidine-2,4,6-triamine;
4,8-bis(4,4-difluoropiperidin-1-yl)-N2,N2,N6,N6-tetrakis(2-methoxyethyl)pyrimido[5,4-d]pyrimidine-2,6-diamine;
N2,N2,N6,N6-tetrakis(2-methoxyethyl)-4-(4-methoxypiperidin-1-yl)-8-(4-(1-methyl-1H-1,2,4-triazol-3-yl)piperazin-1-yl)pyrimido[5,4-d]pyrimidine-2,6-diamine;
3-((6-(bis(2-methoxyethyl)amino)-4,8-bis(4-methoxypiperidin-1-yl)pyrimido[5,4-d]pyrimidin-2-yl)(2-methoxyethyl)amino)propanamide;
4-(2,6-bis(bis(2-methoxyethyl)amino)-8-(4-methoxypiperidin-1-yl)pyrimido[5,4-d]pyrimidin-4-yl)-1-ethylpiperazin-2-one;
4-(2,6-bis(bis(2-ethoxyethyl)amino)-8-(4-methoxypiperidin-1-yl)pyrimido[5,4-d]pyrimidin-4-yl)-1-methylpiperazin-2-one;
4-(4,4-difluoropiperidin-1-yl)-N2,N2,N6,N6-tetrakis(2-methoxyethyl)-8-(4-methoxypiperidin-1-yl)pyrimido[5,4-d]pyrimidine-2,6-diamine;
4,8-bis(4-methoxy-4-methylpiperidin-1-yl)-N2,N2,N6,N6-tetrakis(2-methoxyethyl)pyrimido[5,4-d]pyrimidine-2,6-diamine;
4-(4-(2-methoxyethoxy)piperidin-1-yl)-N2,N2,N6,N6-tetrakis(2-methoxyethyl)-8-(4-methoxypiperidin-1-yl)pyrimido[5,4-d]pyrimidine-2,6-diamine;
3-(((2,6-bis(bis(2-methoxyethyl)amino)-8-(4-methyl-3-oxopiperazin-1-yl)pyrimido[5,4-d]pyrimidin-4-yl)amino)methyl)benzamide;
3-(((2,6-bis(bis(2-methoxyethyl)amino)-8-(4-methyl-3-oxopiperazin-1-yl)pyrimido[5,4-d]pyrimidin-4-yl)amino)methyl)benzenesulfonamide;
3-(((2,6-bis(bis(2-methoxyethyl)amino)-8-(4-methyl-3-oxopiperazin-1-yl)pyrimido[5,4-d]pyrimidin-4-yl)amino)methyl)benzonitrile;
4-(2,6-bis(bis(2-methoxyethyl)amino)-8-(4,4-difluoropiperidin-1-yl)pyrimido[5,4-d]pyrimidin-4-yl)-1-methylpiperazin-2-one;
4-(2,6-bis(bis(2-methoxyethyl)amino)-8-((3-fluorobenzyl)amino)pyrimido[5,4-d]pyrimidin-4-yl)-1-methylpiperazin-2-one;
4-(2,6-bis(bis(2-methoxyethyl)amino)-8-((3-chlorobenzyl)amino)pyrimido[5,4-d]pyrimidin-4-yl)-1-methylpiperazin-2-one;
4-(2,6-bis(bis(2-methoxyethyl)amino)-8-((3-methoxybenzyl)amino)pyrimido[5,4-d]pyrimidin-4-yl)-1-methylpiperazin-2-one;
4-(2,6-bis(bis(2-methoxyethyl)amino)-8-((3-(trifluoromethoxy)benzyl)amino)pyrimido[5,4-d]pyrimidin-4-yl)-1-methylpiperazin-2-one;
5-(((2,6-bis(bis(2-methoxyethyl)amino)-8-(4-methyl-3-oxopiperazin-1-yl)pyrimido[5,4-d]pyrimidin-4-yl)amino)methyl)-2-fluorobenzonitrile;
4-(2,6-bis(bis(2-methoxyethyl)amino)-8-((3-(methylsulfonyl)benzyl)amino)pyrimido[5,4-d]pyrimidin-4-yl)-1-methylpiperazin-2-one;
4-(2,6-bis(bis(2-methoxyethyl)amino)-8-((3,4-difluorobenzyl)amino)pyrimido[5,4-d]pyrimidin-4-yl)-1-methylpiperazin-2-one;
4-(2,6-bis(bis(2-methoxyethyl)amino)-8-(propylamino)pyrimido[5,4-d]pyrimidin-4-yl)-1-methylpiperazin-2-one;
4-(2,6-bis(bis(2-methoxyethyl)amino)-8-(isobutylamino)pyrimido[5,4-d]pyrimidin-4-yl)-1-methylpiperazin-2-one;
4-(2,6-bis(bis(2-methoxyethyl)amino)-8-(4-(1-methyl-1H-1,2,4-triazol-3-yl)piperazin-1-yl)pyrimido[5,4-d]pyrimidin-4-yl)-1-methylpiperazin-2-one;
4-(2,6-bis(bis(2-methoxyethyl)amino)-8-(4-(1-methyl-1H-1,2,4-triazol-3-yl)piperazin-1-yl)pyrimido[5,4-d]pyrimidin-4-yl)thiomorpholine 1,1-dioxide;
N2,N2,N6,N6-tetrakis(2-methoxyethyl)-8-(4-(1-methyl-1H-1,2,4-triazol-3-yl)piperazin-1-yl)-N4-propylpyrimido[5,4-d]pyrimidine-2,4,6-triamine;
N4-isobutyl-N2,N2,N6,N6-tetrakis(2-methoxyethyl)-8-(4-(1-methyl-1H-1,2,4-triazol-3-yl)piperazin-1-yl)pyrimido[5,4-d]pyrimidine-2,4,6-triamine;
N4-(3-fluorobenzyl)-N2,N2,N6,N6-tetrakis(2-methoxyethyl)-8-(4-(1-methyl-1H-1,2,4-triazol-3-yl)piperazin-1-yl)pyrimido[5,4-d]pyrimidine-2,4,6-triamine;
N4-(3-chlorobenzyl)-N2,N2,N6,N6-tetrakis(2-methoxyethyl)-8-(4-(1-methyl-1H-1,2,4-triazol-3-yl)piperazin-1-yl)pyrimido[5,4-d]pyrimidine-2,4,6-triamine;
N4-(3-methoxybenzyl)-N2,N2,N6,N6-tetrakis(2-methoxyethyl)-8-(4-(1-methyl-1H-1,2,4-triazol-3-yl)piperazin-1-yl)pyrimido[5,4-d]pyrimidine-2,4,6-triamine;
N2,N2,N6,N6-tetrakis(2-methoxyethyl)-8-(4-(1-methyl-1H-1,2,4-triazol-3-yl)piperazin-1-yl)-N4-(3-(trifluoromethoxy)benzyl)pyrimido[5,4-d]pyrimidine-2,4,6-triamine;
3-(((2,6-bis(bis(2-methoxyethyl)amino)-8-(4-(1-methyl-1H-1,2,4-triazol-3-yl)piperazin-1-yl)pyrimido[5,4-d]pyrimidin-4-yl)amino)methyl)benzonitrile;
5-(((2,6-bis(bis(2-methoxyethyl)amino)-8-(4-(1-methyl-1H-1,2,4-triazol-3-yl)piperazin-1-yl)pyrimido[5,4-d]pyrimidin-4-yl)amino)methyl)-2-fluorobenzonitrile;
N2,N2,N6,N6-tetrakis(2-methoxyethyl)-8-(4-(1-methyl-1H-1,2,4-triazol-3-yl)piperazin-1-yl)-N4-(3-(methylsulfonyl)benzyl)pyrimido[5,4-d]pyrimidine-2,4,6-triamine;
N4-(3,4-difluorobenzyl)-N2,N2,N6,N6-tetrakis(2-methoxyethyl)-8-(4-(1-methyl-1H-1,2,4-triazol-3-yl)piperazin-1-yl)pyrimido[5,4-d]pyrimidine-2,4,6-triamine;
3-(((2,6-bis(bis(2-methoxyethyl)amino)-8-(4-(1-methyl-1H-1,2,4-triazol-3-yl)piperazin-1-yl)pyrimido[5,4-d]pyrimidin-4-yl)amino)methyl)benzenesulfonamide;
3-(((2,6-bis(bis(2-methoxyethyl)amino)-8-(4-(1-methyl-1H-1,2,4-triazol-3-yl)piperazin-1-yl)pyrimido[5,4-d]pyrimidin-4-yl)amino)methyl)benzamide;
4-(8-(benzyl(methyl)amino)-2,6-bis(bis(2-methoxyethyl)amino)pyrimido[5,4-d]pyrimidin-4-yl)-1-methylpiperazin-2-one;
N2,N2,N6,N6-tetrakis(2-methoxyethyl)-8-(4-methoxypiperidin-1-yl)-N4-methyl-N4-(3-(trifluoromethoxy)benzyl)pyrimido[5,4-d]pyrimidine-2,4,6-triamine;
4-(8-(benzyl(methyl)amino)-2,6-bis(bis(2-ethoxyethyl)amino)pyrimido[5,4-d]pyrimidin-4-yl)-1-methylpiperazin-2-one;
4-(2,6-bis(bis(2-methoxyethyl)amino)-8-((3-fluorobenzyl)(methyl)amino)pyrimido[5,4-d]pyrimidin-4-yl)-1-methylpiperazin-2-one;
4-(2,6-bis(bis(2-methoxyethyl)amino)-8-((3-chlorobenzyl)(methyl)amino)pyrimido[5,4-d]pyrimidin-4-yl)-1-methylpiperazin-2-one;
4-(2,6-bis(bis(2-methoxyethyl)amino)-8-((3-methoxybenzyl)(methyl)amino)pyrimido[5,4-d]pyrimidin-4-yl)-1-methylpiperazin-2-one;
4-(2,6-bis(bis(2-methoxyethyl)amino)-8-(methyl(3-(trifluoromethoxy)benzyl)amino)pyrimido[5,4-d]pyrimidin-4-yl)-1-methylpiperazin-2-one;
3-(((2,6-bis(bis(2-methoxyethyl)amino)-8-(4-methyl-3-oxopiperazin-1-yl)pyrimido[5,4-d]pyrimidin-4-yl)(methyl)amino)methyl)benzonitrile;
5-(((2,6-bis(bis(2-methoxyethyl)amino)-8-(4-methyl-3-oxopiperazin-1-yl)pyrimido[5,4-d]pyrimidin-4-yl)(methyl)amino)methyl)-2-fluorobenzonitrile;
4-(2,6-bis(bis(2-methoxyethyl)amino)-8-((3,4-difluorobenzyl)(methyl)amino)pyrimido[5,4-d]pyrimidin-4-yl)-1-methylpiperazin-2-one;
3-(((2,6-bis(bis(2-methoxyethyl)amino)-8-(4-methyl-3-oxopiperazin-1-yl)pyrimido[5,4-d]pyrimidin-4-yl)(methyl)amino)methyl)benzamide;
N4-(3-fluorobenzyl)-N2,N2,N6,N6-tetrakis(2-methoxyethyl)-N4-methyl-8-(4-(1-methyl-1H-1,2,4-triazol-3-yl)piperazin-1-yl)pyrimido[5,4-d]pyrimidine-2,4,6-triamine;
N4-(3-chlorobenzyl)-N2,N2,N6,N6-tetrakis(2-methoxyethyl)-N4-methyl-8-(4-(1-methyl-1H-1,2,4-triazol-3-yl)piperazin-1-yl)pyrimido[5,4-d]pyrimidine-2,4,6-triamine;
N4-(3-methoxybenzyl)-N2,N2,N6,N6-tetrakis(2-methoxyethyl)-N4-methyl-8-(4-(1-methyl-1H-1,2,4-triazol-3-yl)piperazin-1-yl)pyrimido[5,4-d]pyrimidine-2,4,6-triamine;
3-(((2,6-bis(bis(2-methoxyethyl)amino)-8-(4-(1-methyl-1H-1,2,4-triazol-3-yl)piperazin-1-yl)pyrimido[5,4-d]pyrimidin-4-yl)(methyl)amino)methyl)benzonitrile;
5-(((2,6-bis(bis(2-methoxyethyl)amino)-8-(4-(1-methyl-1H-1,2,4-triazol-3-yl)piperazin-1-yl)pyrimido[5,4-d]pyrimidin-4-yl)(methyl)amino)methyl)-2-fluorobenzonitrile;
N2,N2,N6,N6-tetrakis(2-methoxyethyl)-N4-methyl-8-(4-(1-methyl-1H-1,2,4-triazol-3-yl)piperazin-1-yl)-N4-(3-(methylsulfonyl)benzyl)pyrimido[5,4-d]pyrimidine-2,4,6-triamine;
N4-(3,4-difluorobenzyl)-N2,N2,N6,N6-tetrakis(2-methoxyethyl)-N4-methyl-8-(4-(1-methyl-1H-1,2,4-triazol-3-yl)piperazin-1-yl)pyrimido[5,4-d]pyrimidine-2,4,6-triamine;
3-(((2,6-bis(bis(2-methoxyethyl)amino)-8-(4-(1-methyl-1H-1,2,4-triazol-3-yl)piperazin-1-yl)pyrimido[5,4-d]pyrimidin-4-yl)(methyl)amino)methyl)benzenesulfonamide;
3-(((2,6-bis(bis(2-methoxyethyl)amino)-8-(4-(1-methyl-1H-1,2,4-triazol-3-yl)piperazin-1-yl)pyrimido[5,4-d]pyrimidin-4-yl)(methyl)amino)methyl)benzamide;
4-(2,6-bis(bis(2-methoxyethyl)amino)-8-(5,6-dihydroimidazo[1,5-a]pyrazin-7(8H)-yl)pyrimido[5,4-d]pyrimidin-4-yl)-1-methylpiperazin-2-one;
4-(2,6-bis(bis(2-methoxyethyl)amino)-8-(6,7-dihydro-[1,2,3]triazolo[1,5-a]pyrazin-5(4H)-yl)pyrimido[5,4-d]pyrimidin-4-yl)-1-methylpiperazin-2-one;
4-(2,6-bis(bis(2-methoxyethyl)amino)-8-(2-methyl-2,4,6,7-tetrahydro-5H-pyrazolo[4,3-c]pyridin-5-yl)pyrimido[5,4-d]pyrimidin-4-yl)-1-methylpiperazin-2-one;
4-(2,6-bis(bis(2-methoxyethyl)amino)-8-(1-methyl-1,4,6,7-tetrahydro-5H-imidazo[4,5-c]pyridin-5-yl)pyrimido[5,4-d]pyrimidin-4-yl)-1-methylpiperazin-2-one;
4-(2,6-bis(bis(2-methoxyethyl)amino)-8-(6,7-dihydroisoxazolo[4,3-c]pyridin-5(4H)-yl)pyrimido[5,4-d]pyrimidin-4-yl)-1-methylpiperazin-2-one;
4-(2,6-bis(bis(2-methoxyethyl)amino)-8-(1-methyl-1,4,6,7-tetrahydro-5H-pyrazolo[4,3-c]pyridin-5-yl)pyrimido[5,4-d]pyrimidin-4-yl)-1-methylpiperazin-2-one;
4-(2,6-bis(bis(2-methoxyethyl)amino)-8-(2-methyl-6,7-dihydrooxazolo[4,5-c]pyridin-5(4H)-yl)pyrimido[5,4-d]pyrimidin-4-yl)-1-methylpiperazin-2-one;
4-(2,6-bis(bis(2-methoxyethyl)amino)-8-(5,6-dihydroimidazo[1,2-a]pyrazin-7(8H)-yl)pyrimido[5,4-d]pyrimidin-4-yl)-1-methylpiperazin-2-one;
4-(2,6-bis(bis(2-methoxyethyl)amino)-8-(3,4-dihydropyrrolo[1,2-a]pyrazin-2(1H)-yl)pyrimido[5,4-d]pyrimidin-4-yl)-1-methylpiperazin-2-one;
4-(2,6-bis(bis(2-methoxyethyl)amino)-8-(1,4,5,7-tetrahydro-6H-pyrazolo[3,4-c]pyridin-6-yl)pyrimido[5,4-d]pyrimidin-4-yl)-1-methylpiperazin-2-one;
4-(2,6-bis(bis(2-methoxyethyl)amino)-8-(6,7-dihydropyrazolo[1,5-a]pyrazin-5(4H)-yl)pyrimido[5,4-d]pyrimidin-4-yl)-1-methylpiperazin-2-one;
4-(2,6-bis(bis(2-methoxyethyl)amino)-8-(6,7-dihydroisoxazolo[4,5-c]pyridin-5(4H)-yl)pyrimido[5,4-d]pyrimidin-4-yl)-1-methylpiperazin-2-one;
N,N-bis(2-methoxyethyl)-4,8-bis(4-methoxypiperidin-1-yl)-6-((2-methylpyridin-4-yl)methoxy)pyrimido[5,4-d]pyrimidin-2-amine;
N,N-bis(2-methoxyethyl)-4,8-bis(4-methoxypiperidin-1-yl)-6-((6-methylpyridin-3-yl)methoxy)pyrimido[5,4-d]pyrimidin-2-amine;
3-((6-(bis(2-methoxyethyl)amino)-4,8-bis(4-methoxypiperidin-1-yl)pyrimido[5,4-d]pyrimidin-2-yl)(2-methoxyethyl)amino)propanoic acid;
3-((6-(bis(2-methoxyethyl)amino)-4,8-bis(4-methoxypiperidin-1-yl)pyrimido[5,4-d]pyrimidin-2-yl)(2-hydroxyethyl)amino)propanoic acid;
4-(2,6-bis(bis(2-methoxyethyl)amino)-8-(1-methyl-1,4,5,7-tetrahydro-6H-pyrazolo[3,4-c]pyridin-6-yl)pyrimido[5,4-d]pyrimidin-4-yl)-1-methylpiperazin-2-one;
4-(6-(bis(2-methoxyethyl)amino)-2-(ethyl(2-(pyrrolidin-1-yl)ethyl)amino)-8-(4-methoxypiperidin-1-yl)pyrimido[5,4-d]pyrimidin-4-yl)-1-methylpiperazin-2-one;
4-(6-(bis(2-methoxyethyl)amino)-2-((2-methoxyethyl)(3,4,5-trimethoxybenzyl)amino)-8-(4-methoxypiperidin-1-yl)pyrimido[5,4-d]pyrimidin-4-yl)-1-methylpiperazin-2-one;
4-(2-(bis(2-methoxyethyl)amino)-6-((2-methoxyethyl)(3,4,5-trimethoxybenzyl)amino)-8-(4-methoxypiperidin-1-yl)pyrimido[5,4-d]pyrimidin-4-yl)-1-methylpiperazin-2-one;
4-(2-(bis(2-hydroxyethyl)amino)-6-(bis(2-methoxyethyl)amino)-8-(4-methoxypiperidin-1-yl)pyrimido[5,4-d]pyrimidin-4-yl)-1-methylpiperazin-2-one;
4-(6-(bis(2-hydroxyethyl)amino)-2-(bis(2-methoxyethyl)amino)-8-(4-methoxypiperidin-1-yl)pyrimido[5,4-d]pyrimidin-4-yl)-1-methylpiperazin-2-one;
4-(6-(bis(2-methoxyethyl)amino)-2-(ethyl(2-(piperidin-1-yl)ethyl)amino)-8-(4-methoxypiperidin-1-yl)pyrimido[5,4-d]pyrimidin-4-yl)-1-methylpiperazin-2-one;
4-((6-(bis(2-methoxyethyl)amino)-8-(4-methoxypiperidin-1-yl)-4-(4-methyl-3-oxopiperazin-1-yl)pyrimido[5,4-d]pyrimidin-2-yl)(2-methoxyethyl)amino)butanoic acid;
4-((6-(bis(2-methoxyethyl)amino)-4-(4-methoxypiperidin-1-yl)-8-(4-methyl-3-oxopiperazin-1-yl)pyrimido[5,4-d]pyrimidin-2-yl)(2-methoxyethyl)amino)butanoic acid;
4-(6-(bis(2-methoxyethyl)amino)-2-((2-hydroxyethyl)(2-methoxyethyl)amino)-8-(4-methoxypiperidin-1-yl)pyrimido[5,4-d]pyrimidin-4-yl)-1-methylpiperazin-2-one;
4-(2-(bis(2-methoxyethyl)amino)-6-((2-hydroxyethyl)(2-methoxyethyl)amino)-8-(4-methoxypiperidin-1-yl)pyrimido[5,4-d]pyrimidin-4-yl)-1-methylpiperazin-2-one;
N-(2-((6-(bis(2-methoxyethyl)amino)-8-(4-methoxypiperidin-1-yl)-4-(4-methyl-3-oxopiperazin-1-yl)pyrimido[5,4-d]pyrimidin-2-yl)(2-hydroxyethyl)amino)ethyl)-N-methylacetamide;
4-(2-(bis(2-methoxyethyl)amino)-8-(4-methoxypiperidin-1-yl)-6-(3-oxopiperazin-1-yl)pyrimido[5,4-d]pyrimidin-4-yl)-1-methylpiperazin-2-one;
4-(2-(bis(2-methoxyethyl)amino)-8-(4-methoxypiperidin-1-yl)-6-(4-(methylsulfonyl)piperazin-1-yl)pyrimido[5,4-d]pyrimidin-4-yl)-1-methylpiperazin-2-one;
4-(6-(bis(2-methoxyethyl)amino)-2-((2-hydroxyethyl)(3-(methylsulfonyl)propyl)amino)-8-(4-methoxypiperidin-1-yl)pyrimido[5,4-d]pyrimidin-4-yl)-1-methylpiperazin-2-one;
4-(6-(4-acetylpiperazin-1-yl)-2-(bis(2-methoxyethyl)amino)-8-(4-methoxypiperidin-1-yl)pyrimido[5,4-d]pyrimidin-4-yl)-1-methylpiperazin-2-one;
4-(6-(bis(2-methoxyethyl)amino)-2-(5,6-dihydro-[1,2,4]triazolo[1,5-a]pyrazin-7(8H)-yl)-8-(4-methoxypiperidin-1-yl)pyrimido[5,4-d]pyrimidin-4-yl)-1-methylpiperazin-2-one;
4-(2-(bis(2-methoxyethyl)amino)-6-(5,6-dihydro-[1,2,4]triazolo[1,5-a]pyrazin-7(8H)-yl)-8-(4-methoxypiperidin-1-yl)pyrimido[5,4-d]pyrimidin-4-yl)-1-methylpiperazin-2-one;
methyl 4-(6-(bis(2-methoxyethyl)amino)-4-(4-methoxypiperidin-1-yl)-8-(4-methyl-3-oxopiperazin-1-yl)pyrimido[5,4-d]pyrimidin-2-yl)piperazine-1-carboxylate;
4-(2-((2-(1H-imidazol-1-yl)ethyl)(2-methoxyethyl)amino)-6-(bis(2-methoxyethyl)amino)-8-(4-methoxypiperidin-1-yl)pyrimido[5,4-d]pyrimidin-4-yl)-1-methylpiperazin-2-one;
4-(6-((2-(1H-imidazol-1-yl)ethyl)(2-methoxyethyl)amino)-2-(bis(2-methoxyethyl)amino)-8-(4-methoxypiperidin-1-yl)pyrimido[5,4-d]pyrimidin-4-yl)-1-methylpiperazin-2-one;
4-(6-(bis(2-methoxyethyl)amino)-2-(6,7-dihydro-[1,2,3]triazolo[1,5-a]pyrazin-5(4H)-yl)-8-(4-methoxypiperidin-1-yl)pyrimido[5,4-d]pyrimidin-4-yl)-1-methylpiperazin-2-one;
4-(2-(bis(2-methoxyethyl)amino)-6-(6,7-dihydro-[1,2,3]triazolo[1,5-a]pyrazin-5(4H)-yl)-8-(4-methoxypiperidin-1-yl)pyrimido[5,4-d]pyrimidin-4-yl)-1-methylpiperazin-2-one;
4-(6-(bis(2-methoxyethyl)amino)-2-((2-methoxyethyl)(3-(methylsulfonyl)propyl)amino)-8-(4-methoxypiperidin-1-yl)pyrimido[5,4-d]pyrimidin-4-yl)-1-methylpiperazin-2-one;
4-(6-(bis(2-methoxyethyl)amino)-8-(4-methoxypiperidin-1-yl)-2-(1,4,6,7-tetrahydro-5H-imidazo[4,5-c]pyridin-5-yl)pyrimido[5,4-d]pyrimidin-4-yl)-1-methylpiperazin-2-one;
4-(2-(bis(2-methoxyethyl)amino)-8-(4-methoxypiperidin-1-yl)-6-(1,4,6,7-tetrahydro-5H-imidazo[4,5-c]pyridin-5-yl)pyrimido[5,4-d]pyrimidin-4-yl)-1-methylpiperazin-2-one;
4-(2-(bis(2-methoxyethyl)amino)-8-(4-methoxypiperidin-1-yl)-6-(4-(methylsulfonyl)piperidin-1-yl)pyrimido[5,4-d]pyrimidin-4-yl)-1-methylpiperazin-2-one;
4-(2-(4-(3-aminopyridin-2-yl)piperazin-1-yl)-6-(bis(2-methoxyethyl)amino)-8-(4-methoxypiperidin-1-yl)pyrimido[5,4-d]pyrimidin-4-yl)-1-methylpiperazin-2-one;
4-(6-(4-(3-aminopyridin-2-yl)piperazin-1-yl)-2-(bis(2-methoxyethyl)amino)-8-(4-methoxypiperidin-1-yl)pyrimido[5,4-d]pyrimidin-4-yl)-1-methylpiperazin-2-one;
4-(6-(bis(2-methoxyethyl)amino)-2-(5,6-dihydroimidazo[1,2-a]pyrazin-7(8H)-yl)-8-(4-methoxypiperidin-1-yl)pyrimido[5,4-d]pyrimidin-4-yl)-1-methylpiperazin-2-one;
4-(2-(bis(2-methoxyethyl)amino)-6-(5,6-dihydroimidazo[1,2-a]pyrazin-7(8H)-yl)-8-(4-methoxypiperidin-1-yl)pyrimido[5,4-d]pyrimidin-4-yl)-1-methylpiperazin-2-one;
4-(6-(bis(2-methoxyethyl)amino)-2-(5,6-dihydroimidazo[1,5-a]pyrazin-7(8H)-yl)-8-(4-methoxypiperidin-1-yl)pyrimido[5,4-d]pyrimidin-4-yl)-1-methylpiperazin-2-one;
4-(2-(bis(2-methoxyethyl)amino)-6-(5,6-dihydroimidazo[1,5-a]pyrazin-7(8H)-yl)-8-(4-methoxypiperidin-1-yl)pyrimido[5,4-d]pyrimidin-4-yl)-1-methylpiperazin-2-one;
4-(2-(bis(2-methoxyethyl)amino)-6-(5,6-dihydro-[1,2,4]triazolo[4,3-a]pyrazin-7(8H)-yl)-8-(4-methoxypiperidin-1-yl)pyrimido[5,4-d]pyrimidin-4-yl)-1-methylpiperazin-2-one;
4-(2-(ethyl(2-(pyrrolidin-1-yl)ethyl)amino)-6-((2-methoxyethyl)(3,4,5-trimethoxybenzyl)amino)-8-(4-methoxypiperidin-1-yl)pyrimido[5,4-d]pyrimidin-4-yl)-1-methylpiperazin-2-one;
4-(2,6-bis(bis(2-methoxyethyl)amino)-8-(2-methyl-5,6-dihydro-[1,2,4]triazolo[1,5-a]pyrazin-7(8H)-yl)pyrimido[5,4-d]pyrimidin-4-yl)-1-methylpiperazin-2-one;
4-(6-(bis(2-methoxyethyl)amino)-2-((2-methoxyethyl)(2,3,4-trimethoxybenzyl)amino)-8-(4-methoxypiperidin-1-yl)pyrimido[5,4-d]pyrimidin-4-yl)-1-methylpiperazin-2-one;
4-(2-(bis(2-methoxyethyl)amino)-6-((2-methoxyethyl)(2,3,4-trimethoxybenzyl)amino)-8-(4-methoxypiperidin-1-yl)pyrimido[5,4-d]pyrimidin-4-yl)-1-methylpiperazin-2-one;
4-(2-(bis(2-hydroxyethyl)amino)-6-((2-methoxyethyl)(2,3,4-trimethoxybenzyl)amino)-8-(4-methoxypiperidin-1-yl)pyrimido[5,4-d]pyrimidin-4-yl)-1-methylpiperazin-2-one;
4-(6-(bis(2-hydroxyethyl)amino)-2-((2-methoxyethyl)(2,3,4-trimethoxybenzyl)amino)-8-(4-methoxypiperidin-1-yl)pyrimido[5,4-d]pyrimidin-4-yl)-1-methylpiperazin-2-one;
4-(2,6-bis((2-hydroxyethyl)(2-methoxyethyl)amino)-8-(4-methoxypiperidin-1-yl)pyrimido[5,4-d]pyrimidin-4-yl)-1-methylpiperazin-2-one;
4-(2-(bis(2-hydroxyethyl)amino)-6-(bis(2-methoxyethyl)amino)-8-(4-methoxypiperidin-1-yl)pyrimido[5,4-d]pyrimidin-4-yl)-1-methylpiperazine-2,6-dione;
4-(6-(bis(2-hydroxyethyl)amino)-2-(bis(2-methoxyethyl)amino)-8-(4-methoxypiperidin-1-yl)pyrimido[5,4-d]pyrimidin-4-yl)-1-methylpiperazine-2,6-dione;
4-(2-(bis(2-hydroxyethyl)amino)-6-(bis(2-methoxyethyl)amino)-8-(4-methoxypiperidin-1-yl)pyrimido[5,4-d]pyrimidin-4-yl)-1,6-dimethylpiperazin-2-one;
4-(6-(bis(2-hydroxyethyl)amino)-2-(bis(2-methoxyethyl)amino)-8-(4-methoxypiperidin-1-yl)pyrimido[5,4-d]pyrimidin-4-yl)-1,6-dimethylpiperazin-2-one;
4-(2-(bis(2-hydroxyethyl)amino)-6-(bis(2-methoxyethyl)amino)-8-(4-methoxypiperidin-1-yl)pyrimido[5,4-d]pyrimidin-4-yl)-1,6,6-trimethylpiperazin-2-one;
4-(6-(bis(2-hydroxyethyl)amino)-2-(bis(2-methoxyethyl)amino)-8-(4-methoxypiperidin-1-yl)pyrimido[5,4-d]pyrimidin-4-yl)-1,6,6-trimethylpiperazin-2-one;
2-(2-(bis(2-hydroxyethyl)amino)-6-(bis(2-methoxyethyl)amino)-8-(4-methoxypiperidin-1-yl)pyrimido[5,4-d]pyrimidin-4-yl)hexahydropyrrolo[1,2-a]pyrazin-4(1H)-one;
2-(6-(bis(2-hydroxyethyl)amino)-2-(bis(2-methoxyethyl)amino)-8-(4-methoxypiperidin-1-yl)pyrimido[5,4-d]pyrimidin-4-yl)hexahydropyrrolo[1,2-a]pyrazin-4(1H)-one;
4-(2-(bis(2-hydroxyethyl)amino)-6-(bis(2-methoxyethyl)amino)-8-(4-methoxypiperidin-1-yl)pyrimido[5,4-d]pyrimidin-4-yl)-1-cyclopropylpiperazin-2-one;
4-(6-(bis(2-hydroxyethyl)amino)-2-(bis(2-methoxyethyl)amino)-8-(4-methoxypiperidin-1-yl)pyrimido[5,4-d]pyrimidin-4-yl)-1-cyclopropylpiperazin-2-one;
N2,N2,N6,N6-tetrakis(2-methoxyethyl)-4-(4-methoxypiperidin-1-yl)-8-(piperidin-1-yl)pyrimido[5,4-d]pyrimidine-2,6-diamine;
4-(2,6-bis(bis(2-methoxyethyl)amino)-8-(piperidin-1-yl)pyrimido[5,4-d]pyrimidin-4-yl)-1-methylpiperazin-2-one;
4-(2,6-bis(bis(2-methoxyethyl)amino)-8-(dimethylamino)pyrimido[5,4-d]pyrimidin-4-yl)-1-methylpiperazin-2-one;
4-(2,6-bis(bis(2-hydroxyethyl)amino)-8-(4-methoxypiperidin-1-yl)pyrimido[5,4-d]pyrimidin-4-yl)-1-methylpiperazin-2-one;
2,2′-((6-(bis(2-methoxyethyl)amino)-8-(4-methoxypiperidin-1-yl)-4-(2-methyl-6,7-dihydrooxazolo[4,5-c]pyridin-5(4H)-yl)pyrimido[5,4-d]pyrimidin-2-yl)azanediyl)bis(ethan-1-ol);
2,2′-((6-(bis(2-methoxyethyl)amino)-4-(4-methoxypiperidin-1-yl)-8-(2-methyl-5,6-dihydro-[1,2,4]triazolo[1,5-a]pyrazin-7(8H)-yl)pyrimido[5,4-d]pyrimidin-2-yl)azanediyl)bis(ethan-1-ol);
2,2′-((6-(bis(2-methoxyethyl)amino)-8-((3-fluorobenzyl)amino)-4-(4-methoxypiperidin-1-yl)pyrimido[5,4-d]pyrimidin-2-yl)azanediyl)bis(ethan-1-ol);
3-(((6-(bis(2-hydroxyethyl)amino)-2-(bis(2-methoxyethyl)amino)-8-(4-methoxypiperidin-1-yl)pyrimido[5,4-d]pyrimidin-4-yl)amino)methyl)benzonitrile;
2,2′-((6-(bis(2-methoxyethyl)amino)-8-(3-hydroxy-3-(trifluoromethyl)azetidin-1-yl)-4-(4-methoxypiperidin-1-yl)pyrimido[5,4-d]pyrimidin-2-yl)azanediyl)bis(ethan-1-ol);
4-(2-(bis(2-hydroxypropyl)amino)-6-(bis(2-methoxyethyl)amino)-8-(4-methoxypiperidin-1-yl)pyrimido[5,4-d]pyrimidin-4-yl)-1-methylpiperazin-2-one;
4-(2-(bis(2-hydroxyethyl)amino)-6-(bis(2-methoxypropyl)amino)-8-(4-methoxypiperidin-1-yl)pyrimido[5,4-d]pyrimidin-4-yl)-1-methylpiperazin-2-one;
4-(2-(bis(3-hydroxypropyl)amino)-6-(bis(3-methoxypropyl)amino)-8-(4-methoxypiperidin-1-yl)pyrimido[5,4-d]pyrimidin-4-yl)-1-methylpiperazin-2-one;
3,3′-((6-(bis(3-methoxypropyl)amino)-4,8-bis(4-methoxypiperidin-1-yl)pyrimido[5,4-d]pyrimidin-2-yl)azanediyl)bis(propan-1-ol);
3-((6-(bis(2-methoxyethyl)amino)-8-(4-methoxypiperidin-1-yl)-4-(4-methyl-3-oxopiperazin-1-yl)pyrimido[5,4-d]pyrimidin-2-yl)(2-methoxyethyl)amino)propanoic acid;
3-((6-(bis(2-methoxyethyl)amino)-4-(4-methoxypiperidin-1-yl)-8-(4-methyl-3-oxopiperazin-1-yl)pyrimido[5,4-d]pyrimidin-2-yl)(2-methoxyethyl)amino)propanoic acid;
4-(((6-(bis(2-methoxyethyl)amino)-8-(4-methoxypiperidin-1-yl)-4-(4-methyl-3-oxopiperazin-1-yl)pyrimido[5,4-d]pyrimidin-2-yl)(2-methoxyethyl)amino)methyl)benzoic acid;
3-((6-(bis(2-methoxyethyl)amino)-8-(4-methoxypiperidin-1-yl)-4-(4-methyl-3-oxopiperazin-1-yl)pyrimido[5,4-d]pyrimidin-2-yl)(2-methoxyethyl)amino)-N—(N,N-dimethylsulfamoyl)propenamide;
4-(((6-(bis(2-methoxyethyl)amino)-4-(4-methyl-3-oxopiperazin-1-yl)-8-(2-methyl-6,7-dihydrooxazolo[4,5-c]pyridin-5(4H)-yl)pyrimido[5,4-d]pyrimidin-2-yl)(2-methoxyethyl)amino)methyl)benzoic acid;
4-(((6-(bis(2-methoxyethyl)amino)-8-(4-methoxypiperidin-1-yl)-4-(4-methyl-3-oxopiperazin-1-yl)pyrimido[5,4-d]pyrimidin-2-yl)(2-methoxyethyl)amino)methyl)-2-fluorobenzoic acid;
4-(((6-(bis(2-methoxyethyl)amino)-8-(4-methoxypiperidin-1-yl)-4-(4-methyl-3-oxopiperazin-1-yl)pyrimido[5,4-d]pyrimidin-2-yl)(2-methoxyethyl)amino)methyl)-2-methoxybenzoic acid;
4-(((6-(bis(2-methoxyethyl)amino)-8-(4-methoxypiperidin-1-yl)-4-(4-methyl-3-oxopiperazin-1-yl)pyrimido[5,4-d]pyrimidin-2-yl)(2-methoxyethyl)amino)methyl)-2-methylbenzoic acid;
4-(((6-(bis(2-methoxyethyl)amino)-8-(4-methoxypiperidin-1-yl)-4-(4-methyl-3-oxopiperazin-1-yl)pyrimido[5,4-d]pyrimidin-2-yl)(2-methoxyethyl)amino)methyl)-2-(trifluoromethyl)benzoic acid;
4-(((6-(bis(2-methoxyethyl)amino)-8-(4-methoxypiperidin-1-yl)-4-(4-methyl-3-oxopiperazin-1-yl)pyrimido[5,4-d]pyrimidin-2-yl)(2-methoxyethyl)amino)methyl)-2,6-difluorobenzoic acid,
and pharmaceutically acceptable salts or solvates thereof.

5. A pharmaceutical composition comprising a compound or pharmaceutically acceptable salt or solvate thereof according to claim 1 and at least one pharmaceutically acceptable excipient.

6. (canceled)

7. (canceled)

8. (canceled)

9. (canceled)

10. (canceled)

11. (canceled)

12. (canceled)

13. (canceled)

14. (canceled)

15. (canceled)

16. The pharmaceutical composition according to claim 5, wherein the composition further comprises an adenosine receptor antagonist.

17. The pharmaceutical composition according to claim 16, wherein the adenosine receptor antagonist is an A2A or A2B receptor antagonist.

18. The pharmaceutical composition according to claim 16, wherein the adenosine receptor antagonist is a compound of Formula (II): 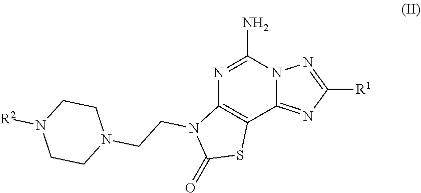
or a pharmaceutically acceptable salt thereof, wherein:
R¹ represents 5- or 6-membered heteroaryl or 5- or 6-membered aryl, wherein heteroaryl or aryl groups are optionally substituted by one or more substituent selected from C₁-C₆ alkyl and halo;
R² represents 6-membered aryl or 6-membered heteroaryl,
wherein heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino and alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl alkylsulfonyl and alkylsulfonealkyl;
or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; each of which are optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

19. The pharmaceutical composition according to claim 16, wherein the adenosine receptor antagonist is antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide;
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
or pharmaceutically acceptable salts thereof.

20. The pharmaceutical composition according to claim 16, wherein the adenosine receptor antagonist is antagonist is:
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one,
or a pharmaceutically acceptable salt thereof.

21. A pharmaceutical composition comprising a compound or pharmaceutically acceptable salt or solvate thereof according to claim 2 and at least one pharmaceutically acceptable excipient.

22. The pharmaceutical composition according to claim 21, wherein the composition further comprises an adenosine receptor antagonist.

23. The pharmaceutical composition according to claim 22, wherein the adenosine receptor antagonist is an A2A or A2B receptor antagonist.

24. The pharmaceutical composition according to claim 22, wherein the adenosine receptor antagonist is a compound of Formula (II): 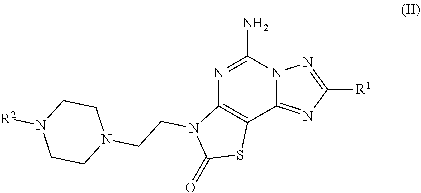
or a pharmaceutically acceptable salt thereof, wherein:
R¹ represents 5- or 6-membered heteroaryl or 5- or 6-membered aryl, wherein heteroaryl or aryl groups are optionally substituted by one or more substituent selected from C₁-C₆ alkyl and halo;
R² represents 6-membered aryl or 6-membered heteroaryl,
wherein heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino and alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl alkylsulfonyl and alkylsulfonealkyl;
or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; each of which are optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

25. The pharmaceutical composition according to claim 22, wherein the adenosine receptor antagonist is antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide;
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
or pharmaceutically acceptable salts thereof.

26. The pharmaceutical composition according to claim 22, wherein the adenosine receptor antagonist is antagonist is:
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one,
or a pharmaceutically acceptable salt thereof.

27. A pharmaceutical composition comprising a compound or pharmaceutically acceptable salt or solvate thereof according to claim 3 and at least one pharmaceutically acceptable excipient.

28. The pharmaceutical composition according to claim 27, wherein the composition further comprises an adenosine receptor antagonist.

29. The pharmaceutical composition according to claim 28, wherein the adenosine receptor antagonist is an A2A or A2B receptor antagonist.

30. The pharmaceutical composition according to claim 28, wherein the adenosine receptor antagonist is a compound of Formula (II): 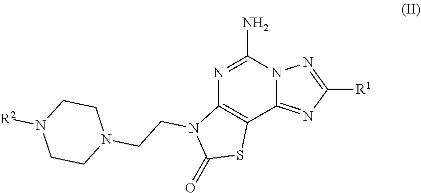
or a pharmaceutically acceptable salt thereof, wherein:
R¹ represents 5- or 6-membered heteroaryl or 5- or 6-membered aryl, wherein heteroaryl or aryl groups are optionally substituted by one or more substituent selected from C₁-C₆ alkyl and halo;
R² represents 6-membered aryl or 6-membered heteroaryl,
wherein heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino and alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl alkylsulfonyl and alkylsulfonealkyl;
or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; each of which are optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

31. The pharmaceutical composition according to claim 28, wherein the adenosine receptor antagonist is antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide;
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
or pharmaceutically acceptable salts thereof.

32. The pharmaceutical composition according to claim 28, wherein the adenosine receptor antagonist is antagonist is:
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one,
or a pharmaceutically acceptable salt thereof.

33. A pharmaceutical composition comprising a compound or pharmaceutically acceptable salt or solvate thereof according to claim 4 and at least one pharmaceutically acceptable excipient.

34. The pharmaceutical composition according to claim 33, wherein the composition further comprises an adenosine receptor antagonist.

35. The pharmaceutical composition according to claim 34, wherein the adenosine receptor antagonist is an A2A or A2B receptor antagonist.

36. The pharmaceutical composition according to claim 34, wherein the adenosine receptor antagonist is a compound of Formula (II): 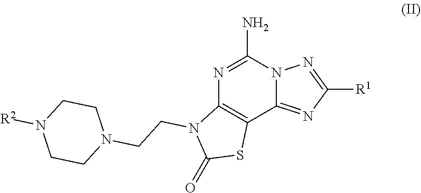
or a pharmaceutically acceptable salt thereof, wherein:
R¹ represents 5- or 6-membered heteroaryl or 5- or 6-membered aryl, wherein heteroaryl or aryl groups are optionally substituted by one or more substituent selected from C₁-C₆ alkyl and halo;
R² represents 6-membered aryl or 6-membered heteroaryl,
wherein heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino and alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl alkylsulfonyl and alkylsulfonealkyl;
or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; each of which are optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

37. The pharmaceutical composition according to claim 34, wherein the adenosine receptor antagonist is antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide;
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
or pharmaceutically acceptable salts thereof.

38. The pharmaceutical composition according to claim 34, wherein the adenosine receptor antagonist is antagonist is:
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one,
or a pharmaceutically acceptable salt thereof.

39. A method of treating cancer in a patient in need thereof, comprising administering to the patient an effective amount of a compound or pharmaceutically acceptable salt or solvate thereof according to claim 1.

40. The method according to claim 39, wherein the method further comprises administering an effective amount of an adenosine receptor antagonist.

41. The method according to claim 40, wherein the adenosine receptor antagonist is (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one, or a pharmaceutically acceptable salt thereof.

42. A method of treating cancer in a patient in need thereof, comprising administering to the patient an effective amount of a compound or pharmaceutically acceptable salt or solvate thereof according to claim 2.

43. The method according to claim 42, wherein the method further comprises administering an effective amount of an adenosine receptor antagonist.

44. The method according to claim 43, wherein the adenosine receptor antagonist is (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one, or a pharmaceutically acceptable salt thereof.

45. A method of treating cancer in a patient in need thereof, comprising administering to the patient an effective amount of a compound or pharmaceutically acceptable salt or solvate thereof according to claim 3.

46. The method according to claim 45, wherein the method further comprises administering an effective amount of an adenosine receptor antagonist.

47. The method according to claim 46, wherein the adenosine receptor antagonist is (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one, or a pharmaceutically acceptable salt thereof.

48. A method of treating cancer in a patient in need thereof, comprising administering to the patient an effective amount of a compound or pharmaceutically acceptable salt or solvate thereof according to claim 4.

49. The method according to claim 48, wherein the method further comprises administering an effective amount of an adenosine receptor antagonist.

50. The method according to claim 49, wherein the adenosine receptor antagonist is (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one, or a pharmaceutically acceptable salt thereof.

51. A kit of parts comprising:
(a) a first part comprising a compound or pharmaceutically acceptable salt or solvate thereof according to claim 1; and
(b) a second part comprising an adenosine receptor antagonist.

52. A kit of parts comprising:
(a) a first part comprising a compound or pharmaceutically acceptable salt or solvate thereof according to claim 2; and
(b) a second part comprising an adenosine receptor antagonist.

53. A kit of parts comprising:
(a) a first part comprising a compound or pharmaceutically acceptable salt or solvate thereof according to claim 3; and
(b) a second part comprising an adenosine receptor antagonist.

54. A kit of parts comprising:
(a) a first part comprising a compound or pharmaceutically acceptable salt or solvate thereof according to claim 4; and
(b) a second part comprising an adenosine receptor antagonist.
